(19) United States
(12) Patent Application Publication
(10) Pub. No.: US 2025/0345325 A1
(43) Pub. Date: Nov. 13, 2025

(54) BIOMARKERS FOR THE DIAGNOSIS AND TREATMENT OF FIBROTIC LUNG DISEASE

(71) Applicant: The Regents of the University of Colorado, A Body Corporate, Denver, CO (US)

(72) Inventors: David A. SCHWARTZ, Aurora, CO (US); Ivana V. YANG, Englewood, CO (US); Joyce S. LEE, Cherry Hills Village, CO (US); Christopher M. EVANS, Denver, CO (US); Marvin I. SCHWARZ, Denver, CO (US)

(21) Appl. No.: 19/278,260

(22) Filed: Jul. 23, 2025

Related U.S. Application Data
(63) Continuation of application No. 17/930,488, filed on Sep. 8, 2022, now Pat. No. 12,396,991.
Application No. 17/930,488 is a continuation of application No. 16/624,500, filed on Dec. 19, 2019, now abandoned.
(60) Provisional application No. 62/525,087, filed on Jun. 26, 2017.
(60) Provisional application No. 62/525,088, filed on Jun. 26, 2017.

(51) Int. Cl. A61K 31/496; A61K 9/00; A61K 31/197; A61K 31/198; A61K 31/4412; A61K 31/4418; A61P 11/00; C12Q 1/6883

(52) U.S. Cl. CPC A61K 31/496; A61K 9/0056; A61K 31/197; A61K 31/4412; A61P 11/00; C12Q 1/6883; A61K 31/198; A61K 31/4418; C12Q 2600/106; C12Q 2600/156

(57) ABSTRACT

The present disclosure provides a method of treating a fibrotic lung disease in a subject comprising administering to the subject an effective amount of a therapeutic agent, wherein the subject is asymptomatic and wherein the subject is at risk of developing the fibrotic lung disease.

### RELATED APPLICATIONS

This application is a continuation of U.S. patent application Ser. No. 17/930,488, filed on Sep. 8, 2022, which is a continuation of U.S. patent application Ser. No. 16/624,500, filed Dec. 19, 2019, which is a National Stage Application, filed under 35 U.S.C. § 371, of PCT/US2018/039573, filed Jun. 26, 2018, which claims the benefit of provisional application U.S. Ser. No. 62/525,087, filed Jun. 26, 2017 and U.S. Ser. No. 62/525,088, filed Jun. 26, 2017, the contents of each of which are herein incorporated by reference in their entirety.

### GOVERNMENT SUPPORT

This invention was made with government support under grant number HL097163, HL123442, and HL138131 awarded by National Institutes of Health and grant number W81XWH-17-1-0597 awarded by Department of Defense. The government has certain rights in the invention.

### INCORPORATION OF SEQUENCE LISTING

The contents of the electronic sequence listing (EL15_001_04US_SeqList_ST26.xml; Size: 227,051 bytes; and Date of Creation: Jul. 23, 2025) are herein incorporated by reference in its entirety.

### FIELD OF THE DISCLOSURE

The disclosure is directed to molecular biology, genetics, and therapeutics for fibrotic lung disease.

### BACKGROUND

Fibrotic pulmonary diseases are progressive and irreversible. Standard therapies are mere palliative as they cannot address the underlying disease mechanism once the subject has progressed to a point at which symptoms are present. Thus, there is a long-felt but unmet need in the field for a method of treating asymptomatic subjects as well as those who are at risk of developing fibrotic pulmonary diseases to prevent onset of the disease, delay onset of the disease, or reduce the severity of disease symptoms, The methods of the disclosure provide a preventative or efficacious treatment, as opposed to a merely palliative treatment, for asymptomatic subjects as well as those subjects at risk of developing the disease.

### SUMMARY

The disclosure provides a method of treating a fibrotic lung disease in a subject comprising administering to the subject an effective amount of a therapeutic agent, wherein the subject is asymptomatic and wherein the subject is at risk of developing the fibrotic lung disease.

In some embodiments of the methods of the disclosure, the subject presents radiographic Usual Interstitial Pneumonia (UIP). In some embodiments, the subject has fibrotic interstitial lung disease (FILD). In some embodiments, the subject has a blood relative with familial interstitial pneumonia (FIP). In some embodiments, including those embodiments wherein the subject has a blood relative with familial interstitial pneumonia (FIP), the blood relative is a sibling. Alternatively, or in addition, in some embodiments, the subject has a mutation in a sequence encoding Mucin 5B (MUC5B), Telomerase RNA Component (TERC), Family with sequence similarity 13 member A (FAM13A), Telomerase Reverse Transcriptase (TERT), Desmoplakin (DSP), Zinc-alpha 2-Glycoprotein 1 (AZGP1), Oligonucleotide/oligosaccharide-binding Fold Containing 1 (OBFC1), ATPase Phospholipid Transporting 11A (ATP11A), Isovaleryl-CoA dehydrogenase (IVD)/Dispatched RND Transporter Family Member 2 (DISP2), Dipeptidyl Peptidase 9 (DPP9), Sialic Acid Binding Ig-Like Lectin 14 (SIGLEC14), Adrenomedullin 2 (ADM2), Tetraspanin 5 (TSPAN5), Calcium/Calmodulin-Dependent Protein Kinase 1 (CAMKK1), zinc finger with KRAB and SCAN domains 1 (ZKSCAN1), isovaleryl-CoA dehydrogenase (IVD), ATPase phospholipid transporting 11A (AK025511) or Matrix Metalloprotease-7 (MMP-7).

In some embodiments of the methods of the disclosure, the subject has a mutation in a sequence encoding MUC5B, TERC, FAM13A, TERT, DSP, AZGP1, OBFC1, ATP11A, IVD/DISP2, DPP9, SIGLEC14, ADM2, TSPAN5, CAMKK1 or MMP-7.

In some embodiments of the methods of the disclosure, the subject has a mutation in a nucleic acid or amino acid sequence encoding a gene or gene product that is upregulated in a subject having a fibrotic pulmonary disease of the disclosure. In some embodiments of the methods of the disclosure, the subject has a mutation in a nucleic acid or amino acid sequence encoding Leukotriene A4 Hydrolase (LTA4H), Surfactant Protein B (SFTPB), Breast Cancer Anti-Estrogen Resistance 3 (BCAR3), C—X—C motif Chemokine Ligand 13 (CXCL13), EPH Receptor A2 (EPHA2), Serum Amyloid A1 (SAA1), Phospholipase A2 Group IIA (PLA2G2A), Insulin-Like Growth Factor Binding Protein 3 (IGFBP3), C—C Motif Chemokine Ligand 28 (CCL28), S100 Calcium Binding Protein A12 (S100A12), Thromboxane A Synthase 1 (TBXAS1), Leukocyte Cell Derived Chemotaxin 1 (LECT1), Complement C3 (C3), Gastrin Releasing Peptide (GRP), C-Reactive Protein (CRP), Vitrin (VIT), Insulin-Like Growth Factor Binding Protein 1 (IGFBP1), Family with Sequence Similarity 173 Member A (FAM173A), Natriuretic Peptide A (NPPA), Secreted Frizzled Related Protein 1 (SFRP1), Ezrin (EZR), Inter-Alpha-Trypsin Inhibitor Heavy Chain Family Member 5 (ITIH5), Pleckstrin and Sec7 Domain Containing 2 (PSD2), Galectin 3 Binding Protein (LGALS3BP), Catenin Beta 1 (CTNNB1), Chromodomain Y Like 2 (CDYL2), Matrix Metallopeptidase 7 (MMP7), Apolipoprotein B (APOB), Proline and Arginine Rich End Leucine Rich Repeat Protein (PRELP), Eukaryotic Translation Initiation Factor 1A, X-linked (EIF1AX), Mesencephalic Astrocyte Derived Neurotrophic Factor (MANF), TNF Receptor Superfamily Member 13C (TNFRSF13C), Deformed Epidermal Autoregulatory Factor 1 transcription factor (DEAF1), Tumor Protein Translationally-Controlled 1 (TPT1), Unc-5 Netrin Receptor B (UNC5B), Phosphatidylethanolamine Binding Protein 1 (PEBP1), Syntaxin 8 (STX8), Polymeric Immunoglobulin Receptor (PIGR), Adenine Phosphoribosyltransferase (APRT), Matrix Metallopeptidase 3 (MMP3), Galectin 7 (LGALS7), Bruton Tyrosine Kinase (BTK), NSFL1 Cofactor (NSFL1C), FER Tyrosine Kinase (FER), Regenerating Family Member 1 Beta (REG1B), SMAD Family Member 2 (SMAD2), Interleukin 1 Receptor Like 1 (IL1RL1), C—C Motif Chemokine Ligand 18 (CCL18), Acid Phosphatase 2 Lysosomal (ACP2), Eukaryotic Translation Initiation Factor 4E Family Member 2 (EIF4E2), Neurexin 3 (NRXN3), IGF Like Family Member 1 (IGFL1), NME/NM23 Nucleoside Diphosphate Kinase 1 (NME1), Potassium Voltage-Gated Channel Isk-Related Family Member 1-Like (KCNE1L) or Neurexophilin 2 (NXPH2).

In some embodiments of the methods of the disclosure, the subject has a mutation in a nucleic acid or amino acid sequence encoding a gene or gene product that is downregulated in a subject having a fibrotic pulmonary disease of the disclosure. In some embodiments of the methods of the disclosure, the subject has a mutation in a nucleic acid or amino acid sequence encoding Surfactant Protein D (SFTPD), Glyceraldehyde-3-Phosphate Dehydrogenase (GAPDH), Histone Cluster 1 H1 Family Member C (HIST1H1C), YTH Domain Containing 1 (YTHDC1), Plexin A1 (PLXNA1), Serine Peptidase Inhibitor Kazal Type 6 (SPINK6), LDL Receptor Related Protein Associated Protein 1 (LRPAP1), Secretoglobin Family 3A Member 1 (SCGB3A1), H2A Histone Family Member Z (H2AFZ) or Chromosome 1 Open Reading Frame 162 (C1orf162).

In some embodiments of the methods of the disclosure, the subject has a mutation in a sequence encoding MUC5B. In some embodiments, the mutation is a polymorphism in a sequence encoding a MUC5B promoter. In some embodiments, the polymorphism is rs35705950 comprising (SEQ ID NO: 7).

In some embodiments of the methods of the disclosure, the human subject has a mutation in a sequence encoding TERC. In some embodiments, the mutation is a polymorphism in a sequence encoding TERC or a regulatory sequence thereof. In some embodiments the polymorphism is rs6793295 comprising (SEQ ID NO: 1).

In some embodiments of the methods of the disclosure, the human subject has a mutation in a sequence encoding intronic FAM13A. In some embodiments, the mutation is a polymorphism in a sequence encoding intronic FAM13A or a regulatory sequence thereof. In some embodiments, the polymorphism is rs2609260.

In some embodiments of the methods of the disclosure, the human subject has a mutation in a sequence encoding intronic TERT. In some embodiments, the mutation is a polymorphism in a sequence encoding intronic TERT or a regulatory sequence thereof. In some embodiments, the polymorphism is rs4449583.

In some embodiments of the methods of the disclosure, the human subject has a mutation in a sequence encoding intronic DSP. In some embodiments, the mutation is a polymorphism in a sequence encoding intronic DSP or a regulatory sequence thereof. In some embodiments, the polymorphism is rs2076295.

In some embodiments of the methods of the disclosure, the human subject has a mutation in a sequence encoding intronic ZKSCAN1. In some embodiments, the mutation is a polymorphism in a sequence encoding intronic ZKSCAN1 or a regulatory sequence thereof. In some embodiments, the polymorphism is rs6963345.

In some embodiments of the methods of the disclosure, the human subject has a mutation in a sequence encoding intronic OBFC1. In some embodiments, the mutation is a polymorphism in a sequence encoding intronic OBFC1 or a regulatory sequence thereof. In some embodiments, the polymorphism is rs2488000.

In some embodiments of the methods of the disclosure, the human subject has a mutation in a sequence encoding an AK025511 3′ UTR. In some embodiments, the mutation is a polymorphism in a sequence encoding an AK025511 3′ UTR or a regulatory sequence thereof. In some embodiments, the polymorphism is rs1278769.

In some embodiments of the methods of the disclosure, the human subject has a mutation in a sequence encoding IVD. In some embodiments, the mutation is a polymorphism in a sequence encoding intronic IVD or a regulatory sequence thereof. In some embodiments, the polymorphism is rs35700143.

In some embodiments of the methods of the disclosure, the human subject has a mutation in a sequence encoding intronic DPP9. In some embodiments, the mutation is a polymorphism in a sequence encoding intronic DPP9 or a regulatory sequence thereof. In some embodiments, the polymorphism is rs12610495.

In some embodiments of the methods of the disclosure, the subject has a mutation in a sequence encoding FAM13A. In some embodiments, the mutation is a polymorphism in a sequence encoding FAM13A or a regulatory sequence thereof. In some embodiments the polymorphism is rs2609255 comprising (SEQ ID NO: 2).

In some embodiments of the methods of the disclosure, the subject has a mutation in a sequence encoding TERT. In some embodiments, the mutation is a polymorphism in a sequence encoding TERT or a regulatory sequence thereof. In some embodiments the polymorphism is rs2736100 comprising (SEQ ID NO: 3).

In some embodiments of the methods of the disclosure, the subject has a mutation in a sequence encoding DSP. In some embodiments, the mutation is a polymorphism in a sequence encoding DSP or a regulatory sequence thereof. In some embodiments the polymorphism is rs2076295 comprising (SEQ ID NO: 4).

In some embodiments of the methods of the disclosure, the subject has a mutation in a sequence encoding AZGP1. In some embodiments, the mutation is a polymorphism in a sequence encoding AZGP1 or a regulatory sequence thereof. In some embodiments the polymorphism is rs4727443 comprising (SEQ ID NO: 5).

In some embodiments of the methods of the disclosure, the subject has a mutation in a sequence encoding OBFC1. In some embodiments, the mutation is a polymorphism in a sequence encoding OBFC1 or a regulatory sequence thereof. In some embodiments the polymorphism is rs11191865 comprising (SEQ ID NO: 6).

In some embodiments of the methods of the disclosure, the subject has a mutation in a sequence encoding ATP11A. In some embodiments, the mutation is a polymorphism in a sequence encoding ATP11A or a regulatory sequence thereof. In some embodiments the polymorphism is rs12787690 comprising (SEQ ID NO: 8).

In some embodiments of the methods of the disclosure, the subject has a mutation in a sequence encoding IVD/DISP2. In some embodiments, the mutation is a polymorphism in a sequence encoding IVD/DISP2 or a regulatory sequence thereof. In some embodiments the polymorphism is rs2034650 comprising (SEQ ID NO: 9).

In some embodiments of the methods of the disclosure, the subject has a mutation in a sequence encoding DPP9. In some embodiments, the mutation is a polymorphism in a sequence encoding DPP9 or a regulatory sequence thereof. In some embodiments the polymorphism is rs12610495 comprising (SEQ ID NO: 10).

In some embodiments of the methods of the disclosure, the fibrotic lung disease is pulmonary fibrosis, idiopathic pulmonary fibrosis (IPF), an interstitial lung abnormality (ILA), or an asymptomatic ILA. In some embodiments, the fibrotic lung disease is pulmonary fibrosis or IPF. In some embodiments, the fibrotic lung disease is IPF.

In some embodiments of the methods of the disclosure, the therapeutic agent comprises a N-acetylcysteine, pirfenidone, and nintedanib.

In some embodiments of the methods of the disclosure, the therapeutic agent comprises pirfenidone. In some embodiments, the effective dosage is administered orally as a capsule or a tablet. In some embodiments, including those embodiments wherein the therapeutic agent comprises pirfenidone, the effective dosage is about 2400 mg/day. In some embodiments, the effective dosage is administered according to an escalating dosage regimen. In some embodiments, including those embodiments wherein the therapeutic agent comprises pirfenidone, the escalating dosage regimen comprises (a) administering to the subject about 800 mg of pirfenidone per day for a first week; (b) administering to the subject about 1600 mg of pirfenidone per day for a second week; and (c) administering to the subject about 2400 mg of pirfenidone per day for the remainder of the treatment. In some embodiments, including those embodiments wherein the therapeutic agent comprises pirfenidone, the escalating dosage regimen comprises (a) administering to the subject a capsule or tablet comprising about 250 mg of pirfenidone three times a day for a first week; (b) administering to the subject two capsules or tablets comprising about 250 mg of pirfenidone three times a day for a second week; and (c) administering to the subject three capsules or tablets comprising about 250 mg of pirfenidone three times a day for the remainder of the treatment. In some embodiments of the escalating dosage regimen, the capsule or tablet comprises 267 mg of pirfenidone.

In some embodiments of the methods of the disclosure, the therapeutic agent comprises nintedanib. In some embodiments, the effective dosage is administered orally as a capsule or a tablet. In some embodiments, including those embodiments wherein the therapeutic agent comprises nintedanib, the effective dosage is about 300 mg/day. In some embodiments, the effective dosage is about 150 mg administered twice per day, wherein the daily doses are administered about 12 hours apart from one another. In some embodiments, including those embodiments wherein the therapeutic agent comprises nintedanib, the effective dosage is about 200 mg/day. In some embodiments, the effective dosage is about 100 mg administered twice per day, wherein the daily doses are administered about 12 hours apart from one another. In some embodiments, including those embodiments wherein the therapeutic agent comprises nintedanib, the effective dosage is administered according to a modified or interrupted dosage regimen. In some embodiments, the modified or interrupted dosage regimen comprises (a) administering to the subject about 300 mg of nintedanib per day until the subject presents an elevated level of liver enzymes compared to a control level of liver enzymes; (b) administering to the subject about 200 mg of nintedanib per day until the subject presents the control level of liver enzymes; and (c) administering to the subject about 300 mg of nintedanib per day for the remainder of the treatment; wherein the control level of liver enzymes is a level detected in the subject prior to an initiation of the treatment. In some embodiments, including those embodiments wherein the therapeutic agent comprises nintedanib, the modified or interrupted regimen comprises (a) administering to the subject a capsule or tablet comprising about 150 mg of nintedanib twice per day until the subject presents an elevated level of liver enzymes compared to a control level of liver enzymes; (b) administering to the subject two capsules or tablets comprising about 100 mg twice per day until the subject presents an elevated level of liver enzymes compared to a control level of liver enzymes; and (c) administering to the subject a capsule or tablet comprising about 150 mg of nintedanib twice per day for the remainder of the treatment; wherein the control level of liver enzymes is a level detected in the subject prior to an initiation of the treatment.

In some embodiments of the methods of the disclosure, the therapeutic agent prevents the onset or development of a sign or symptom of the fibrotic lung disease.

In some embodiments of the methods of the disclosure, the therapeutic agent delays the onset or development of a sign or symptom of the fibrotic lung disease when compared to the expected onset of the sign or symptom in the absence of treatment with the therapeutic agent.

In some embodiments of the methods of the disclosure, the therapeutic agent reduces the severity of a sign or symptom of the fibrotic lung disease when compared to the expected severity of the sign or symptom in the absence of treatment with the therapeutic agent.

In some embodiments of the methods of the disclosure, the therapeutic agent reduces the severity of a sign or symptom of the fibrotic lung disease when compared to the expected severity of the sign or symptom in the absence of treatment with the therapeutic agent.

In some embodiments of the methods of the disclosure, the at least one sign of the fibrotic lung disease is detectable before the subject presents a symptom of the fibrotic lung disease. In some embodiments, the at least one sign comprises gradual or unintended weight loss, clubbing of the fingers or toes, rapid and shallow breathing, fibrotic lesions in one or both lungs detectable by radiography, or a cough. In some embodiments, the symptom comprises shortness of breath during exercise, shortness of breath at rest, a dry and hacking cough, repeated bouts of coughing, and uncontrollable bouts of coughing.

In some embodiments of the methods of the disclosure, the method prevents the onset of a secondary condition associated with a severe form of the fibrotic lung disease. In some embodiments, a secondary condition comprises a collapsed lung, an infected lung, a blood clot in a lung, lung cancer, respiratory failure, pulmonary hypertension, heart failure or death.

The disclosure provides a method of identifying a therapeutic agent or target thereof for the treatment of a fibrotic lung disease, comprising administering to a non-human subject a dose of a composition that modifies transcription or translation of a sequence encoding Mucin 5B (MUC5B), Telomerase RNA Component (TERC), Family with sequence similarity 13 member A (FAM13A), Telomerase Reverse Transcriptase (TERT), Desmoplakin (DSP), Zinc-alpha 2-Glycoprotein 1 (AZGP1), Oligonucleotide/oligosaccharide-binding Fold Containing 1 (OBFC1), ATPase Phospholipid Transporting 11A (ATP11A), Isovaleryl-CoA dehydrogenase (IVD)/Dispatched RND Transporter Family Member 2 (DISP2), Dipeptidyl Peptidase 9 (DPP9), Sialic Acid Binding Ig-Like Lectin 14 (SIGLEC14), Adrenomedullin 2 (ADM2), Tetraspanin 5 (TSPAN5), Calcium/Calmodulin-Dependent Protein Kinase Kinase 1 (CAMKK1) or Matrix Metalloprotease-7 (MMP-7), wherein the dose of the composition is tolerable to the non-human subject and wherein the dose of the composition is therapeutically effective.

In some embodiments of the methods of identifying a therapeutic agent or target thereof for the treatment of a fibrotic lung disease of the disclosure, the method of identifying a therapeutic agent or target thereof for the treatment of a fibrotic lung disease, comprising administering to a non-human subject a composition that modifies an activity of a product of a sequence encoding MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, wherein the dose of the composition is tolerable to the non-human subject and wherein the dose of the composition is therapeutically effective.

In some embodiments of the methods of identifying a therapeutic agent or target thereof for the treatment of a fibrotic lung disease of the disclosure, the composition that modifies transcription or translation decreases or inhibits transcription or translation.

In some embodiments of the methods of identifying a therapeutic agent or target thereof for the treatment of a fibrotic lung disease of the disclosure, the composition decreases or inhibits transcription or translation of a sequence encoding a gene selected from the group consisting of Leukotriene A4 Hydrolase (LTA4H), Surfactant Protein B (SFTPB), Breast Cancer Anti-Estrogen Resistance 3 (BCAR3), C—X—C motif Chemokine Ligand 13 (CXCL13), EPH Receptor A2 (EPHA2), Serum Amyloid A1 (SAA1), Phospholipase A2 Group IIA (PLA2G2A), Insulin-Like Growth Factor Binding Protein 3 (IGFBP3), C—C Motif Chemokine Ligand 28 (CCL28), S100 Calcium Binding Protein A12 (S100A12), Thromboxane A Synthase 1 (TBXAS1), Leukocyte Cell Derived Chemotaxin 1 (LECT1), Complement C3 (C3), Gastrin Releasing Peptide (GRP), C-Reactive Protein (CRP), Vitrin (VIT), Insulin-Like Growth Factor Binding Protein 1 (IGFBP1), Family with Sequence Similarity 173 Member A (FAM173A), Natriuretic Peptide A (NPPA), Secreted Frizzled Related Protein 1 (SFRP1), Ezrin (EZR), Inter-Alpha-Trypsin Inhibitor Heavy Chain Family Member 5 (ITIH5), Pleckstrin and Sec7 Domain Containing 2 (PSD2), Galectin 3 Binding Protein (LGALS3BP), Catenin Beta 1 (CTNNB1), Chromodomain Y Like 2 (CDYL2), Matrix Metallopeptidase 7 (MMP7), Apolipoprotein B (APOB), Proline and Arginine Rich End Leucine Rich Repeat Protein (PRELP), Eukaryotic Translation Initiation Factor 1A, X-linked (EIF1AX), Mesencephalic Astrocyte Derived Neurotrophic Factor (MANF), TNF Receptor Superfamily Member 13C (TNFRSF13C), Deformed Epidermal Autoregulatory Factor 1 transcription factor (DEAF1), Tumor Protein Translationally-Controlled 1 (TPT1), Unc-5 Netrin Receptor B (UNC5B), Phosphatidylethanolamine Binding Protein 1 (PEBP1), Syntaxin 8 (STX8), Polymeric Immunoglobulin Receptor (PIGR), Adenine Phosphoribosyltransferase (APRT), Matrix Metallopeptidase 3 (MMP3), Galectin 7 (LGALS7), Bruton Tyrosine Kinase (BTK), NSFL1 Cofactor (NSFL1C), FER Tyrosine Kinase (FER), Regenerating Family Member 1 Beta (REG1B), SMAD Family Member 2 (SMAD2), Interleukin 1 Receptor Like 1 (IL1RL1), C—C Motif Chemokine Ligand 18 (CCL18), Acid Phosphatase 2 Lysosomal (ACP2), Eukaryotic Translation Initiation Factor 4E Family Member 2 (EIF4E2), Neurexin 3 (NRXN3), IGF Like Family Member 1 (IGFL1), NME/NM23 Nucleoside Diphosphate Kinase 1 (NME1), Potassium Voltage-Gated Channel Isk-Related Family Member 1-Like (KCNE1L) or Neurexophilin 2 (NXPH2).

In some embodiments of the methods of identifying a therapeutic agent or target thereof for the treatment of a fibrotic lung disease of the disclosure, the composition that modifies transcription or translation increases or activates transcription or translation.

In some embodiments of the methods of identifying a therapeutic agent or target thereof for the treatment of a fibrotic lung disease of the disclosure, the composition increases or activates transcription or translation of a sequence encoding a gene selected from the group consisting of Surfactant Protein D (SFTPD), Glyceraldehyde-3-Phosphate Dehydrogenase (GAPDH), Histone Cluster 1 H1 Family Member C (HIST1H1C), YTH Domain Containing 1 (YTHDC1), Plexin A1 (PLXNA1), Serine Peptidase Inhibitor Kazal Type 6 (SPINK6), LDL Receptor Related Protein Associated Protein 1 (LRPAP1), Secretoglobin Family 3A Member 1 (SCGB3A1), H2A Histone Family Member Z (H2AFZ) or Chromosome 1 Open Reading Frame 162 (C1orf162).

In some embodiments of the methods of identifying a therapeutic agent or target thereof for the treatment of a fibrotic lung disease of the disclosure, the composition that modifies an activity decreases or inhibits the activity.

In some embodiments of the methods of identifying a therapeutic agent or target thereof for the treatment of a fibrotic lung disease of the disclosure, the composition decreases or inhibits the activity of a sequence encoding a gene selected from Leukotriene A4 Hydrolase (LTA4H), Surfactant Protein B (SFTPB), Breast Cancer Anti-Estrogen Resistance 3 (BCAR3), C—X—C motif Chemokine Ligand 13 (CXCL13), EPH Receptor A2 (EPHA2), Serum Amyloid A1 (SAA1), Phospholipase A2 Group IIA (PLA2G2A), Insulin-Like Growth Factor Binding Protein 3 (IGFBP3), C—C Motif Chemokine Ligand 28 (CCL28), S100 Calcium Binding Protein A12 (S100A12), Thromboxane A Synthase 1 (TBXAS1), Leukocyte Cell Derived Chemotaxin 1 (LECT1), Complement C3 (C3), Gastrin Releasing Peptide (GRP), C-Reactive Protein (CRP), Vitrin (VIT), Insulin-Like Growth Factor Binding Protein 1 (IGFBP1), Family with Sequence Similarity 173 Member A (FAM173A), Natriuretic Peptide A (NPPA), Secreted Frizzled Related Protein 1 (SFRP1), Ezrin (EZR), Inter-Alpha-Trypsin Inhibitor Heavy Chain Family Member 5 (ITIH5), Pleckstrin and Sec7 Domain Containing 2 (PSD2), Galectin 3 Binding Protein (LGALS3BP), Catenin Beta 1 (CTNNB1), Chromodomain Y Like 2 (CDYL2), Matrix Metallopeptidase 7 (MMP7), Apolipoprotein B (APOB), Proline and Arginine Rich End Leucine Rich Repeat Protein (PRELP), Eukaryotic Translation Initiation Factor 1A, X-linked (EIF1AX), Mesencephalic Astrocyte Derived Neurotrophic Factor (MANF), TNF Receptor Superfamily Member 13C (TNFRSF13C), Deformed Epidermal Autoregulatory Factor 1 transcription factor (DEAF1), Tumor Protein Translationally-Controlled 1 (TPT1), Unc-5 Netrin Receptor B (UNC5B), Phosphatidylethanolamine Binding Protein 1 (PEBP1), Syntaxin 8 (STX8), Polymeric Immunoglobulin Receptor (PIGR), Adenine Phosphoribosyltransferase (APRT), Matrix Metallopeptidase 3 (MMP3), Galectin 7 (LGALS7), Bruton Tyrosine Kinase (BTK), NSFL1 Cofactor (NSFL1C), FER Tyrosine Kinase (FER), Regenerating Family Member 1 Beta (REG1B), SMAD Family Member 2 (SMAD2), Interleukin 1 Receptor Like 1 (IL1RL1), C—C Motif Chemokine Ligand 18 (CCL18), Acid Phosphatase 2 Lysosomal (ACP2), Eukaryotic Translation Initiation Factor 4E Family Member 2 (EIF4E2), Neurexin 3 (NRXN3), IGF Like Family Member 1 (IGFL1), NME/NM23 Nucleoside Diphosphate Kinase 1 (NME1), Potassium Voltage-Gated Channel Isk-Related Family Member 1-Like (KCNE1L) or Neurexophilin 2 (NXPH2).

In some embodiments of the methods of identifying a therapeutic agent or target thereof for the treatment of a fibrotic lung disease of the disclosure, the composition that modifies an activity increases or activates the activity.

In some embodiments of the methods of identifying a therapeutic agent or target thereof for the treatment of a fibrotic lung disease of the disclosure, the composition increases or activates the activity of a sequence encoding Surfactant Protein D (SFTPD), Glyceraldehyde-3-Phosphate Dehydrogenase (GAPDH), Histone Cluster 1 H1 Family Member C (HIST1H1C), YTH Domain Containing 1 (YTHDC1), Plexin A1 (PLXNA1), Serine Peptidase Inhibitor Kazal Type 6 (SPINK6), LDL Receptor Related Protein Associated Protein 1 (LRPAP1), Secretoglobin Family 3A Member 1 (SCGB3A1), H2A Histone Family Member Z (H2AFZ) or Chromosome 1 Open Reading Frame 162 (C1orf162).

In some embodiments of the methods of identifying a therapeutic agent or target thereof for the treatment of a fibrotic lung disease of the disclosure, the non-human subject is a mammal.

In some embodiments of the methods of identifying a therapeutic agent or target thereof for the treatment of a fibrotic lung disease of the disclosure, the mammal is genetically-modified.

In some embodiments of the methods of the disclosure, the genetically-modified mammal is a model organism for the fibrotic lung disease.

In some embodiments of the methods of identifying a therapeutic agent or target thereof for the treatment of a fibrotic lung disease of the disclosure, the fibrotic lung disease is pulmonary fibrosis, idiopathic pulmonary fibrosis (IPF), an interstitial lung abnormality (ILA), or an asymptomatic ILA.

In some embodiments of the methods of identifying a therapeutic agent or target thereof for the treatment of a fibrotic lung disease of the disclosure, the fibrotic lung disease is pulmonary fibrosis or IPF.

In some embodiments of the methods of identifying a therapeutic agent or target thereof for the treatment of a fibrotic lung disease of the disclosure, the fibrotic lung disease is IPF.

In some embodiments of the methods of identifying a therapeutic agent or target thereof for the treatment of a fibrotic lung disease of the disclosure, the non-human subject carries a mutation in a sequence encoding MUC5B.

In some embodiments of the methods of identifying a therapeutic agent or target thereof for the treatment of a fibrotic lung disease of the disclosure, the mutation comprises a polymorphism in a sequence encoding a MUC5B promoter.

In some embodiments of the methods of identifying a therapeutic agent or target thereof for the treatment of a fibrotic lung disease of the disclosure, the polymorphism is rs35705950.

In some embodiments of the methods of identifying a therapeutic agent or target thereof for the treatment of a fibrotic lung disease of the disclosure, the non-human subject carries a mutation in a sequence encoding TERC, FAM13A, TERT, DSP, ZKSCAN1, AZGP1, OBFC1, MUC5B, AK025511, ATP11A, IVD/DISP2, DPP9, SIGLEC14, ADM2, TSPAN5, CAMKK1 or MMP-7.

In some embodiments of the methods of identifying a therapeutic agent or target thereof for the treatment of a fibrotic lung disease of the disclosure, the composition prevents the onset or development of a sign or symptom of the fibrotic lung disease.

In some embodiments of the methods of identifying a therapeutic agent or target thereof for the treatment of a fibrotic lung disease of the disclosure, the composition delays the onset or development of a sign or symptom of the fibrotic lung disease when compared to the expected onset of the sign or symptom in the absence of treatment with the composition.

In some embodiments of the methods of identifying a therapeutic agent or target thereof for the treatment of a fibrotic lung disease of the disclosure, the composition delays the onset or development of a sign or symptom of the fibrotic lung disease when compared to the expected onset of the sign or symptom when treated using a standard therapeutic intervention.

In some embodiments of the methods of identifying a therapeutic agent or target thereof for the treatment of a fibrotic lung disease of the disclosure, the composition reduces the severity of a sign or symptom of the fibrotic lung disease when compared to the expected severity of the sign or symptom in the absence of treatment with the composition.

In some embodiments of the methods of identifying a therapeutic agent or target thereof for the treatment of a fibrotic lung disease of the disclosure, the composition reduces the severity of a sign or symptom of the fibrotic lung disease when compared to the expected severity of the sign or symptom when treated using a standard therapeutic intervention.

In some embodiments of the methods of identifying a therapeutic agent or target thereof for the treatment of a fibrotic lung disease of the disclosure, the standard therapeutic intervention comprises a N-acetylcysteine, pirfenidone, and nintedanib.

In some embodiments of the methods of identifying a therapeutic agent or target thereof for the treatment of a fibrotic lung disease of the disclosure, the standard therapeutic intervention comprises pirfenidone.

In some embodiments of the methods of identifying a therapeutic agent or target thereof for the treatment of a fibrotic lung disease of the disclosure, an effective dosage of pirfenidone is about 2400 mg/day.

In some embodiments of the methods of identifying a therapeutic agent or target thereof for the treatment of a fibrotic lung disease of the disclosure, the effective dosage is administered orally as a capsule or a tablet.

In some embodiments of the methods of identifying a therapeutic agent or target thereof for the treatment of a fibrotic lung disease of the disclosure, the effective dosage is administered three times per day.

In some embodiments of the methods of identifying a therapeutic agent or target thereof for the treatment of a fibrotic lung disease of the disclosure, the effective dosage is administered according to an escalating dosage regimen.

In some embodiments of the methods of identifying a therapeutic agent or target thereof for the treatment of a fibrotic lung disease of the disclosure, the escalating dosage regimen comprises, administering to the non-human subject about 800 mg of pirfenidone per day for a first week; administering to the non-human subject about 1600 mg of pirfenidone per day for a second week; and administering to the non-human subject about 2400 mg of pirfenidone per day for the remainder of the treatment.

In some embodiments of the methods of identifying a therapeutic agent or target thereof for the treatment of a fibrotic lung disease of the disclosure, the escalating dosage regimen comprises, administering to the non-human subject a capsule or tablet comprising about 250 mg of pirfenidone three times a day for a first week; administering to the non-human subject two capsules or tablets comprising about 250 mg of pirfenidone three times a day for a second week; and administering to the non-human subject three capsules or tablets comprising about 250 mg of pirfenidone three times a day for the remainder of the treatment.

In some embodiments of the methods of identifying a therapeutic agent or target thereof for the treatment of a fibrotic lung disease of the disclosure, the capsule or tablet comprises 267 mg of pirfenidone.

In some embodiments of the methods of identifying a therapeutic agent or target thereof for the treatment of a fibrotic lung disease of the disclosure, the standard therapeutic intervention comprises nintedanib.

In some embodiments of the methods of identifying a therapeutic agent or target thereof for the treatment of a fibrotic lung disease of the disclosure, an effective dosage of nintedanib is administered orally as a capsule or a tablet.

In some embodiments of the methods of identifying a therapeutic agent or target thereof for the treatment of a fibrotic lung disease of the disclosure, the effective dosage is about 300 mg/day.

In some embodiments of the methods of identifying a therapeutic agent or target thereof for the treatment of a fibrotic lung disease of the disclosure, the effective dosage is about 150 mg administered twice per day, wherein the daily doses are administered about 12 hours apart from one another.

In some embodiments of the methods of identifying a therapeutic agent or target thereof for the treatment of a fibrotic lung disease of the disclosure, the effective dosage is about 200 mg/day.

In some embodiments of the methods of identifying a therapeutic agent or target thereof for the treatment of a fibrotic lung disease of the disclosure, the effective dosage is about 100 mg administered twice per day, wherein the daily doses are administered about 12 hours apart from one another.

In some embodiments of the methods of identifying a therapeutic agent or target thereof for the treatment of a fibrotic lung disease of the disclosure, the non-human subject presents at least one sign of the fibrotic lung disease.

In some embodiments of the methods of identifying a therapeutic agent or target thereof for the treatment of a fibrotic lung disease of the disclosure, the at least one sign comprises gradual or unintended weight loss, clubbing of the fingers or toes, rapid and shallow breathing, fibrotic lesions in one or both lungs detectable by radiography, or a cough.

In some embodiments of the methods of identifying a therapeutic agent or target thereof for the treatment of a fibrotic lung disease of the disclosure, the compound prevents the onset of a secondary condition associated with a severe form of the fibrotic lung disease.

In some embodiments of the methods of identifying a therapeutic agent or target thereof for the treatment of a fibrotic lung disease of the disclosure, the compound prevents the onset for at 1 year, 2 years, 3 years, 4 years, 5 years or any whole or fractional number of years in between.

In some embodiments of the methods of identifying a therapeutic agent or target thereof for the treatment of a fibrotic lung disease of the disclosure, secondary condition comprises a collapsed lung, an infected lung, a blood clot in a lung, lung cancer, respiratory failure, pulmonary hypertension, heart failure or death.

The disclosure provides a composition for the treatment of a fibrotic lung disease identified by a method of the disclosure, including, a method of identifying a therapeutic agent or target thereof for the treatment of a fibrotic lung disease of the disclosure.

The disclosure provides a method of treating fibrotic lung disease in a human subject of the disclosure comprising administering a therapeutically effective amount of a composition identified by a method of the disclosure, wherein the subject is asymptomatic and wherein the subject is at risk of developing the fibrotic lung disease. In some embodiments, the subject is wild type (e.g. does not comprises a mutation or a sequence variation) with respect to a nucleic acid or amino acid sequence encoding one or more of TERC, FAM13A, TERT, DSP, ZKSCAN1, AZGP1, OBFC1, MUC5B, AK025511, ATP11A, IVD/DISP2, DPP9, SIGLEC14, ADM2, TSPAN5, CAMKK1 or MMP-7.

In some embodiments of the methods of treating fibrotic lung disease in a human subject of the disclosure by administering a composition identified by a method of the disclosure, the human subject presents radiographic Usual Interstitial Pneumonia (UIP).

In some embodiments of the methods of treating fibrotic lung disease in a human subject of the disclosure by administering a composition identified by a method of the disclosure, wherein the human subject has fibrotic interstitial lung disease (FILD).

In some embodiments of the methods of treating fibrotic lung disease in a human subject of the disclosure by administering a composition identified by a method of the disclosure, wherein the human subject has a blood relative with familial interstitial pneumonia (FIP).

In some embodiments of the methods of treating fibrotic lung disease in a human subject of the disclosure by administering a composition identified by a method of the disclosure, wherein the blood relative is a sibling.

In some embodiments of the methods of treating fibrotic lung disease in a human subject of the disclosure by administering a composition identified by a method of the disclosure, wherein the human subject has a mutation or a sequence variation in a nucleic acid or an amino acid sequence encoding TERC, FAM13A, TERT, DSP, ZKSCAN1, AZGP1, OBFC1, MUC5B, AK025511, ATP11A, IVD/DISP2, DPP9, SIGLEC14, ADM2, TSPAN5, CAMKK1 or MMP-7.

In some embodiments of the methods of treating fibrotic lung disease in a human subject of the disclosure by administering a composition identified by a method of the disclosure, the mutation comprises a polymorphism in a sequence encoding a MUC5B promoter.

In some embodiments of the methods of treating fibrotic lung disease in a human subject of the disclosure by administering a composition identified by a method of the disclosure, the polymorphism is rs35705950.

In some embodiments of the methods of treating fibrotic lung disease in a human subject of the disclosure by administering a composition identified by a method of the disclosure, the fibrotic lung disease is pulmonary fibrosis, idiopathic pulmonary fibrosis (IPF), an interstitial lung abnormality (ILA), or an asymptomatic ILA.

In some embodiments of the methods of treating fibrotic lung disease in a human subject of the disclosure by administering a composition identified by a method of the disclosure, the fibrotic lung disease is pulmonary fibrosis or IPF.

In some embodiments of the methods of treating fibrotic lung disease in a human subject of the disclosure by administering a composition identified by a method of the disclosure, the fibrotic lung disease is IPF.

In some embodiments of the methods of treating fibrotic lung disease in a human subject of the disclosure by administering a composition identified by a method of the disclosure, the method prevents the onset of a secondary condition associated with a severe form of the fibrotic lung disease.

In some embodiments of the methods of treating fibrotic lung disease in a human subject of the disclosure by administering a composition identified by a method of the disclosure, a secondary condition comprises a collapsed lung, an infected lung, a blood clot in a lung, lung cancer, respiratory failure, pulmonary hypertension, heart failure or death.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a map depicting an exemplary hierarchical clustering of differentially expressed genes for pre-pulmonary fibrosis subjects and normal subjects.

FIG. 2A-B is a pair of volcano plots showing serum sample quality control using Principal component analysis (PCA). FIG. 2A shows before outlier exclusion and FIG. 2B shows after outlier exclusion.

FIG. 3 is a volcano plot of 3315 plasma proteins, comparing results from 70 patients with established IPF and 70 controls. Solid red symbols represent 57 proteins that were significantly up-regulated and solid blue symbols 12 proteins that were significantly downregulated in patients with IPF after controlling for multiple comparisons and age/gender/smoking.

FIG. 4 is a survival plot showing receiver operator curves of predictive model for PrePF in asymptomatic relatives from FIP families. Area Under Curve (AUC) values for each model are as follows: Gene Expression alone (red)=0.83, Clinical Predictors (blue)=0.87, Clinical Predictors+MUC5B genotype (green)=0.87, Clinical Predictors+Gene Expression Score (yellow)=0.95, Clinical Predictors+MUC5B genotype+Gene Expression Score (black)=0.95, indicating that a peripheral blood biomarker panel may improve the diagnostic power of a predictive model for PrePF in an at-risk population.

FIG. 5 is a graph showing MUC5B expression in IPF (N=203) and unaffected subjects (N=139) stratified by MUC5B promoter variant (rs35705950) genotype.

FIG. 6A is a microscopic image demonstrating that MUC5B is produced in bronchoalveolar epithelia of patients with IPF (brown staining in photomicrographs). Staining is increased in the airways of patients positive for rs35705950 (TT) compared to WT (GG).

FIG. 6B is a graph showing the percentage of MUC5B positive area of bronchiolar epithelium. Unbiased stereological assessment of staining demonstrates that the volume fraction of stained airways (% positive area) is significantly greater in both the GT heterozygotes and the TT homozygotes.

FIG. 7A-B is series of bar graphs showing that Scgb1a1- and SFPTC promoter show significant worsening of fibrosis (hydroxyproline) after bleomycin while Muc5b−/− mice are protected. FIG. 7A is a series of graphs and FIG. 7B is a series of confocal images showing that the concentration of Muc5b is directly related to the fibroproliferative response to bleomycin. Representative images from second harmonic generation (SHG) demonstrate increased lung collagen (red) in transgenic mice following bleomycin injury.

FIG. 8 is bar graph showing that the baseline expression of ER stress genes in lung tissue from WT and Scgb1a1 Muc5bTg mice. Muc5bTg mice have greater ER stress gene expression than their WT littermates (all genes in the ER stress pathway, with p<0.05). Bleomycin also induces ER stress (data not shown).

FIG. 9 is a pair of microscopic images showing enhanced CHOP (Ddit3) protein in wild type (WT, top photograph) and Scgb1a1-Muc5bTg mice (bottom photograph) after repeat bleomycin.

FIG. 10 is a pair of microscopic images and corresponding graphs showing the expanded mucus layer and decreased mucociliary transport in SFTPC-Muc5bTg mice compared to littermate wild-type mice. Statistical differences were assessed by Mann-Whitney U Test.

FIG. 11 is a series of schematic diagram showing that the MUC5B variant and other biomarkers can identify an at-risk population or those with PrePF, establishing the opportunity for primary and secondary prevention of IPF. The ‘at-risk’ population and the population with PrePF is large (19% with the MUC5B promoter variant and 1.8% of individuals ≥50 years of age respectively), IPF is diagnosed in a small population with established, end-stage disease and PrePF can be identified using the MUC5B variant rs35705950. Results indicate that PrePF (detected via chest CT scan) is associated with a poor prognosis suggesting that PrePF may be a harbinger of IPF.

FIG. 12 is a schematic diagram showing a method of screening at-risk populations (family members of patients with IPF) to identify individuals with PrePF. Focus is placed on identifying the genetic variants and biomarkers that increase the yield of PrePF on HRCT scan, in addition to gender, age, and physiology scores.

FIG. 13 is a table describing the baseline characteristics of patients with rheumatoid arthritis.

FIG. 14 is a table describing the genotypic association of MUC5B rs35705950 single nucleotide polymorphism in patients with RA, with and without interstitial lung disease

FIG. 15 is a table describing the dominant genotypic association of MUC5B rs35705950 single nucleotide polymorphism in patients with RA-ILD and a usual interstitial pneumonia or possible usual interstitial pneumonia pattern (RA-UIP) and in patients with RA-ILD and a pattern inconsistent with usual interstitial pneumonia (RA non-UIP).

FIG. 16A is a forest plot of odds ratios (OR) and 95% confidence intervals {Cl) depicting the lack of association of the MUCSB rs35705950 promoter variant with RA without lLD {RA-nolLD). The boxes indicate OR, and the horizontal lines indicate 95% Cl for the best-fitting genetic model for each association test. The black dotted line represents a mean OR value of 1. The red boxes and red lines indicate the overall OR and 95% Cl, respectively. For comparisons between RA cases and controls, the associations were adjusted for the country of origin and sex. For intra-RA cases comparisons, the associations were adjusted for the country of origin, sex, age at inclusion and smoking.

FIG. 16B is a forest plot of odds ratios (OR) and 95% confidence intervals {Cl) depicting the additive genotypic association of the MUCSB rs 35705950 promoter variant with RA-ILD. The red dotted line represent the mean value of overall OR value. The boxes indicate OR, and the horizontal lines indicate 95% Cl for the best-fitting genetic model for each association test. The black dotted line represents a mean OR value of 1. The red boxes and red lines indicate the overall OR and 95% Cl, respectively. For comparisons between RA cases and controls, the associations were adjusted for the country of origin and sex. For intra-RA cases comparisons, the associations were adjusted for the country of origin, sex, age at inclusion and smoking.

FIG. 16C is a forest plot of odds ratios (OR) and 95% confidence intervals {Cl) depicting dominant genotypic association of the MUCSB re35705950 promoter variant with ILD among patients with RA and those with the usual interstitial pneumonia or possible usual interstitial pneumonia (UIP) pattern. The boxes indicate OR, and the horizontal lines indicate 95% Cl for the best-fitting genetic model for each association test. The red dotted line represent the mean value of overall OR value. The black dotted line represents a mean OR value of 1. The red boxes and red lines indicate the overall OR and 95% Cl, respectively. For comparisons between RA cases and controls, the associations were adjusted for the country of origin and sex. For intra-RA cases comparisons, the associations were adjusted for the country of origin, sex, age at inclusion and smoking.

FIG. 17 is a series of photographs depicting MUC5B expression in explanted lung tissue from rheumatoid arthritis associates interstitial lung disease. Representative lung tissue images from unaffected control (GG genotype, Panel A), RA-ILD case #1 (GG genotype, Panel B), and RA-ILD case #2 (GT genotype, Panel C). Low power views with high power view insets identified. Panel A—low power view of normal lung; top and middle insets with high power view of bronchiole with MUC5B staining; bottom inset with high power view of alveolar epithelia. Panel B and C—low power view of the usual interstitial pneumonia pattern in explanted lung tissue of RA-ILD; top inset with high power view of bronchiole with MUC5B staining; middle and bottom insets with high power view of MUC5B staining in metaplastic epithelia lining honeycomb cysts and MUC5B staining of mucous in honeycomb cysts.

FIG. 18 is a flow chart depicting the screening and enrollment process for study subjects.

FIG. 19A-D is a series of photographs depicting High-resolution CT (HRCT) images of: 19A) chest from a study subject whose scan was read as normal, without signs of interstitial lung disease or fibrosis. 19B) HRCT image from subject who was categorized as having “Probable Fibrotic ILD.” 19C) Representative HRCT image from subject who was characterized as having “Definite Fibrotic ILD.” 19D) HRCT image from a case of previously diagnosed, established Idiopathic Pulmonary Fibrosis (IPF) in one of the study families.

FIG. 20 is a table depicting a summary of characteristics of study subjects used in quantitative CT Analyses.

FIG. 21A-F is a series of photographs depicting representative axial HRCT images visually assessed as “No Fibrosis” (21A), “Probable Fibrotic ILD” (21C) and “Definite Fibrotic ILD” (E). Below each is the corresponding quantitative HRCT results for the above scan: (21B) “No Fibrosis” fibrosis extent 1 7% (fibrosis score=0.55), (21D) “Probable Fibrotic ILD” fibrosis extent 18.5% (fibrosis score 2.92), (F) “Definite Fibrotic ILD” fibrosis extent 35.5% (fibrosis score 3.60), Classification results color coded as follows: green=normal lung, blue=airway, yellow=reticular abnormality, magenta=ground glass opacity, red=honeycombing.

FIG. 22 is a table depicting Screening Cohort Subject Characteristics. * DNA available on a total of 489 subjects (404 No Fibrosis and 75 PrePF subjects). ** Odds ratios reported in this table were calculated from a mixed effects logistic regression model including age (as a continuous variable), male sex, ever smoker (yes/no), and MUC5B promoter variant (rs35705950) genotype. ***In the reported model, rs35705950 coded as a dominant allele; in log-additive genetic model, p=0.05, as well.

FIG. 23 is a table depicting patterns of CT abnormalities in scans with probable or definite fibrotic ILD. * Because a confident single diagnosis was relatively uncommon, most cases included consideration of several patterns. For this reason, the percentages add up to more than 100%.

FIG. 24 is a box plot depicting fibrosis score by visual diagnosis. Boxplots of fibrosis scores based on quantitative HRCT assessment for each visual diagnosis category. Fibrosis score means were significantly different (ANOVA, p<0.0001) across groups defined by visual diagnosis. Comparison of fibrosis score between groups showed significant differences for all comparisons (p<0.01 for all).

FIG. 25A-C is a series of graphs depicting Receiver Operating Characteristic (ROC) curves for quantitative imaging measures of Fibrosis and PrePF. FIG. 5A depicts ROC curves for visual diagnosis compared to log HAA scores. FIG. 5B depicts ROC Curves for visual diagnosis compared to fibrosis scores. ROC analysis showed that fibrosis score discriminates subjects with visual diagnosis of PrePF. Average area under the curve (AUC) in fivefold cross validation was 0.85 (range 0.83-0.87) and average accuracy, sensitivity, and specificity in the test partitions were 0.83 (range 0.74-0.86), 0.74 (range 0.56-0.92) and 0.84 (range 0.76-0.89) respectively. Optimal threshold for fibrosis score ranged from 1.40-1.42. FIG. 5C depicts Density plots of fibrosis scores for visually diagnosed PrePF (pink) and No Fibrosis (blue) scans—the fibrosis score optimal threshold is indicated with the red line (1.40).

FIG. 26 is a series of tables depicting Dyspnea questionnaire data. FIG. 26A depicts breathlessness responses for the cohort. FIG. 26B depicts breathlessness responses by Visual CT diagnosis.

FIG. 27 is a graph that depicts the prevalence of PrePF in FIP Siblings Cohort by Age and MUC5B Genotype. PrePF prevalence in this FIP siblings cohort increases by age, as shown in this graph. By age >60 years, the prevalence of PrePF differed significantly based on MUC5B genotype (*p=0.02). Subjects with the variant are depicted by the red line, while those without it are depicted with the blue line.

FIG. 28 is a table depicting subject characteristics based on Quantitative Fibrosis Score. Clinical characteristics and genotype breakdown of subjects with quantitative HRCT analyses. The cutoff of 1.4 for the logarithm of fibrosis score is based on analyses presented in the text. * p-value compares characteristic between groups. Linear regression values regress fibrosis score on age, male sex, smoking history, and MUC5B promoter variant. **In the reported model, rs35705950 coded as a dominant allele given small number of TT subjects.

FIG. 29 is a table depicting an exploratory genetic association study of 13 pulmonary fibrosis susceptibility variants in RA-ILD.

### DETAILED DESCRIPTION OF THE DISCLOSURE

The present disclosure provides a method of treating a fibrotic lung disease in a subject comprising administering to the subject an effective amount of a therapeutic agent, wherein the subject is asymptomatic and wherein the subject is at risk of developing the fibrotic lung disease.

### Methods of Identifying a Therapeutic Agent of the Disclosure or Target Thereof

The disclosure provides a method of identifying a therapeutic agent or target thereof for the treatment of a fibrotic lung disease, comprising administering to a non-human subject a dose of a composition that modifies transcription or translation of a sequence encoding Mucin 5B (MUC5B), Telomerase RNA Component (TERC), Family with sequence similarity 13 member A (FAM13A), Telomerase Reverse Transcriptase (TERT), Desmoplakin (DSP), Zinc-alpha 2-Glycoprotein 1 (AZGP1), Oligonucleotide/oligosaccharide-binding Fold Containing 1 (OBFC1), ATPase Phospholipid Transporting 11A (ATP11A), Isovaleryl-CoA dehydrogenase (IVD)/Dispatched RND Transporter Family Member 2 (DISP2), Dipeptidyl Peptidase 9 (DPP9), Sialic Acid Binding Ig-Like Lectin 14 (SIGLEC14), Adrenomedullin 2 (ADM2), Tetraspanin 5 (TSPAN5), Calcium/Calmodulin-Dependent Protein Kinase Kinase 1 (CAMKK1) or Matrix Metalloprotease-7 (MMP-7), wherein the dose of the composition is tolerable to the non-human subject and wherein the dose of the composition is therapeutically effective.

The disclosure provides method of identifying a therapeutic agent or target thereof for the treatment of a fibrotic lung disease, comprising administering to a non-human subject a composition that modifies an activity of a product of a sequence encoding MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, wherein the dose of the composition is tolerable to the non-human subject and wherein the dose of the composition is therapeutically effective.

In some embodiments of the methods of the disclosure, the composition that modifies transcription or translation decreases or inhibits transcription or translation. In some embodiments, the composition decreases or inhibits transcription or translation of a sequence encoding a gene selected from the group consisting of Leukotriene A4 Hydrolase (LTA4H), Surfactant Protein B (SFTPB), Breast Cancer Anti-Estrogen Resistance 3 (BCAR3), C—X—C motif Chemokine Ligand 13 (CXCL13), EPH Receptor A2 (EPHA2), Serum Amyloid A1 (SAA1), Phospholipase A2 Group IIA (PLA2G2A), Insulin-Like Growth Factor Binding Protein 3 (IGFBP3), C—C Motif Chemokine Ligand 28 (CCL28), S100 Calcium Binding Protein A12 (S100A12), Thromboxane A Synthase 1 (TBXAS1), Leukocyte Cell Derived Chemotaxin 1 (LECT1), Complement C3 (C3), Gastrin Releasing Peptide (GRP), C-Reactive Protein (CRP), Vitrin (VIT), Insulin-Like Growth Factor Binding Protein 1 (IGFBP1), Family with Sequence Similarity 173 Member A (FAM173A), Natriuretic Peptide A (NPPA), Secreted Frizzled Related Protein 1 (SFRP1), Ezrin (EZR), Inter-Alpha-Trypsin Inhibitor Heavy Chain Family Member 5 (ITIH5), Pleckstrin and Sec7 Domain Containing 2 (PSD2), Galectin 3 Binding Protein (LGALS3BP), Catenin Beta 1 (CTNNB1), Chromodomain Y Like 2 (CDYL2), Matrix Metallopeptidase 7 (MMP7), Apolipoprotein B (APOB), Proline and Arginine Rich End Leucine Rich Repeat Protein (PRELP), Eukaryotic Translation Initiation Factor 1A, X-linked (EIF1AX), Mesencephalic Astrocyte Derived Neurotrophic Factor (MANF), TNF Receptor Superfamily Member 13C (TNFRSF13C), Deformed Epidermal Autoregulatory Factor 1 transcription factor (DEAF1), Tumor Protein Translationally-Controlled 1 (TPT1), Unc-5 Netrin Receptor B (UNC5B), Phosphatidylethanolamine Binding Protein 1 (PEBP1), Syntaxin 8 (STX8), Polymeric Immunoglobulin Receptor (PIGR), Adenine Phosphoribosyltransferase (APRT), Matrix Metallopeptidase 3 (MMP3), Galectin 7 (LGALS7), Bruton Tyrosine Kinase (BTK), NSFL1 Cofactor (NSFL1C), FER Tyrosine Kinase (FER), Regenerating Family Member 1 Beta (REG1B), SMAD Family Member 2 (SMAD2), Interleukin 1 Receptor Like 1 (IL1RL1), C—C Motif Chemokine Ligand 18 (CCL18), Acid Phosphatase 2 Lysosomal (ACP2), Eukaryotic Translation Initiation Factor 4E Family Member 2 (EIF4E2), Neurexin 3 (NRXN3), IGF Like Family Member 1 (IGFL1), NME/NM23 Nucleoside Diphosphate Kinase 1 (NME1), Potassium Voltage-Gated Channel Isk-Related Family Member 1-Like (KCNE1L) or Neurexophilin 2 (NXPH2).

In some embodiments of the methods of the disclosure, the composition that modifies transcription or translation increases or activates transcription or translation. In some embodiments, the composition increases or activates transcription or translation of a sequence encoding a gene selected from the group consisting of Surfactant Protein D (SFTPD), Glyceraldehyde-3-Phosphate Dehydrogenase (GAPDH), Histone Cluster 1 H1 Family Member C (HIST1H1C), YTH Domain Containing 1 (YTHDC1), Plexin A1 (PLXNA1), Serine Peptidase Inhibitor Kazal Type 6 (SPINK6), LDL Receptor Related Protein Associated Protein 1 (LRPAP1), Secretoglobin Family 3A Member 1 (SCGB3A1), H2A Histone Family Member Z (H2AFZ) or Chromosome 1 Open Reading Frame 162 (C1orf162).

In some embodiments of the methods of the disclosure, the composition that modifies an activity decreases or inhibits the activity. In some embodiments, the composition decreases or inhibits the activity of a sequence encoding a gene selected from Leukotriene A4 Hydrolase (LTA4H), Surfactant Protein B (SFTPB), Breast Cancer Anti-Estrogen Resistance 3 (BCAR3), C—X—C motif Chemokine Ligand 13 (CXCL13), EPH Receptor A2 (EPHA2), Serum Amyloid A1 (SAA1), Phospholipase A2 Group IIA (PLA2G2A), Insulin-Like Growth Factor Binding Protein 3 (IGFBP3), C—C Motif Chemokine Ligand 28 (CCL28), S100 Calcium Binding Protein A12 (S100A12), Thromboxane A Synthase 1 (TBXAS1), Leukocyte Cell Derived Chemotaxin 1 (LECT1), Complement C3 (C3), Gastrin Releasing Peptide (GRP), C-Reactive Protein (CRP), Vitrin (VIT), Insulin-Like Growth Factor Binding Protein 1 (IGFBP1), Family with Sequence Similarity 173 Member A (FAM173A), Natriuretic Peptide A (NPPA), Secreted Frizzled Related Protein 1 (SFRP1), Ezrin (EZR), Inter-Alpha-Trypsin Inhibitor Heavy Chain Family Member 5 (ITIH5), Pleckstrin and Sec7 Domain Containing 2 (PSD2), Galectin 3 Binding Protein (LGALS3BP), Catenin Beta 1 (CTNNB1), Chromodomain Y Like 2 (CDYL2), Matrix Metallopeptidase 7 (MMP7), Apolipoprotein B (APOB), Proline and Arginine Rich End Leucine Rich Repeat Protein (PRELP), Eukaryotic Translation Initiation Factor 1A, X-linked (EIF1AX), Mesencephalic Astrocyte Derived Neurotrophic Factor (MANF), TNF Receptor Superfamily Member 13C (TNFRSF13C), Deformed Epidermal Autoregulatory Factor 1 transcription factor (DEAF1), Tumor Protein Translationally-Controlled 1 (TPT1), Unc-5 Netrin Receptor B (UNC5B), Phosphatidylethanolamine Binding Protein 1 (PEBP1), Syntaxin 8 (STX8), Polymeric Immunoglobulin Receptor (PIGR), Adenine Phosphoribosyltransferase (APRT), Matrix Metallopeptidase 3 (MMP3), Galectin 7 (LGALS7), Bruton Tyrosine Kinase (BTK), NSFL1 Cofactor (NSFL1C), FER Tyrosine Kinase (FER), Regenerating Family Member 1 Beta (REG1B), SMAD Family Member 2 (SMAD2), Interleukin 1 Receptor Like 1 (IL1RL1), C—C Motif Chemokine Ligand 18 (CCL18), Acid Phosphatase 2 Lysosomal (ACP2), Eukaryotic Translation Initiation Factor 4E Family Member 2 (EIF4E2), Neurexin 3 (NRXN3), IGF Like Family Member 1 (IGFL1), NME/NM23 Nucleoside Diphosphate Kinase 1 (NME1), Potassium Voltage-Gated Channel Isk-Related Family Member 1-Like (KCNE1L) or Neurexophilin 2 (NXPH2).

In some embodiments of the methods of the disclosure, the composition that modifies an activity increases or activates the activity. In some embodiments, the composition increases or activates the activity of a sequence encoding Surfactant Protein D (SFTPD), Glyceraldehyde-3-Phosphate Dehydrogenase (GAPDH), Histone Cluster 1 H1 Family Member C (HIST1H1C), YTH Domain Containing 1 (YTHDC1), Plexin A1 (PLXNA1), Serine Peptidase Inhibitor Kazal Type 6 (SPINK6), LDL Receptor Related Protein Associated Protein 1 (LRPAP1), Secretoglobin Family 3A Member 1 (SCGB3A1), H2A Histone Family Member Z (H2AFZ) or Chromosome 1 Open Reading Frame 162 (C1orf162).

In some embodiments of the methods of the disclosure, the non-human subject is a mammal. In some embodiments, mammal is genetically-modified. In some embodiments, the genetically-modified mammal is a model organism for the fibrotic lung disease.

In some embodiments of the methods of the disclosure, the fibrotic lung disease is pulmonary fibrosis, idiopathic pulmonary fibrosis (IPF), an interstitial lung abnormality (ILA), or an asymptomatic ILA. In some embodiments, the fibrotic lung disease is pulmonary fibrosis or IPF. In some embodiments, the fibrotic lung disease is IPF.

In some embodiments of the methods of the disclosure, the non-human subject carries a mutation in a sequence encoding MUC5B. In some embodiments, the mutation comprises a polymorphism in a sequence encoding a MUC5B promoter. In some embodiments, the polymorphism is rs35705950. Alternatively, or in addition, in some embodiments, the non-human subject carries a mutation in a sequence encoding TERC, FAM13A, TERT, DSP, AZGP1, OBFC1, ATP11A, IVD/DISP2, DPP9, SIGLEC14, ADM2, TSPAN5, CAMKK1 or MMP-7.

In some embodiments of the methods of the disclosure, the composition prevents the onset or development of a sign or symptom of the fibrotic lung disease.

In some embodiments of the methods of the disclosure, the composition delays the onset or development of a sign or symptom of the fibrotic lung disease when compared to the expected onset of the sign or symptom in the absence of treatment with the composition. In some embodiments, the composition delays the onset or development of a sign or symptom of the fibrotic lung disease when compared to the expected onset of the sign or symptom when treated using a standard therapeutic intervention.

In some embodiments of the methods of the disclosure, the composition reduces the severity of a sign or symptom of the fibrotic lung disease when compared to the expected severity of the sign or symptom in the absence of treatment with the composition. In some embodiments, the composition reduces the severity of a sign or symptom of the fibrotic lung disease when compared to the expected severity of the sign or symptom when treated using a standard therapeutic intervention.

In some embodiments of the methods of the disclosure, the standard therapeutic intervention comprises a N-acetylcysteine, pirfenidone, and nintedanib.

In some embodiments of the methods of the disclosure, the standard therapeutic intervention comprises pirfenidone. In some embodiments, an effective dosage of pirfenidone is about 2400 mg/day. In some embodiments, the effective dosage is administered orally as a capsule or a tablet. In some embodiments, the effective dosage is administered three times per day. In some embodiments, the effective dosage is administered according to an escalating dosage regimen. In some embodiments, the escalating dosage regimen comprises (a) administering to the non-human subject about 800 mg of pirfenidone per day for a first week; (b) administering to the non-human subject about 1600 mg of pirfenidone per day for a second week; and (c) administering to the non-human subject about 2400 mg of pirfenidone per day for the remainder of the treatment. In some embodiments, the escalating dosage regimen comprises (a) administering to the non-human subject a capsule or tablet comprising about 250 mg of pirfenidone three times a day for a first week; (b) administering to the non-human subject two capsules or tablets comprising about 250 mg of pirfenidone three times a day for a second week; and (c) administering to the non-human subject three capsules or tablets comprising about 250 mg of pirfenidone three times a day for the remainder of the treatment. In some embodiments, the capsule or tablet comprises 267 mg of pirfenidone.

In some embodiments of the methods of the disclosure, the standard therapeutic intervention comprises nintedanib. In some embodiments, an effective dosage of nintedanib is administered orally as a capsule or a tablet. In some embodiments, the effective dosage is about 300 mg/day. In some embodiments, the effective dosage is about 150 mg administered twice per day, wherein the daily doses are administered about 12 hours apart from one another. In some embodiments, the effective dosage is about 200 mg/day. In some embodiments, the effective dosage is about 100 mg administered twice per day, wherein the daily doses are administered about 12 hours apart from one another.

In some embodiments of the methods of the disclosure, the non-human subject presents at least one sign of the fibrotic lung disease. In some embodiments, the at least one sign comprises gradual or unintended weight loss, clubbing of the fingers or toes, rapid and shallow breathing, fibrotic lesions in one or both lungs detectable by radiography, or a cough.

In some embodiments of the methods of the disclosure, the compound prevents the onset of a secondary condition associated with a severe form of the fibrotic lung disease. In some embodiments, the compound prevents the onset for at 1 year, 2 years, 3 years, 4 years, 5 years or any whole or fractional number of years in between. In some embodiments, the secondary condition comprises a collapsed lung, an infected lung, a blood clot in a lung, lung cancer, respiratory failure, pulmonary hypertension, heart failure or death.

The disclosure provides a composition for the treatment of a fibrotic lung disease identified by a method of the disclosure for identifying a therapeutic agent or target thereof for the treatment of a fibrotic lung disease.

### Subjects of the Disclosure

The disclosure provides a method of treating a fibrotic lung disease in a human subject comprising administering to the subject the composition for the treatment of a fibrotic lung disease identified by a method of the disclosure for identifying a therapeutic agent or target thereof for the treatment of a fibrotic lung disease, wherein the subject is asymptomatic and wherein the subject is at risk of developing the fibrotic lung disease.

In some embodiments of the methods of treating a fibrotic lung disease in a human subject of the disclosure, the human subject presents radiographic Usual Interstitial Pneumonia (UIP). In some embodiments, the human subject has fibrotic interstitial lung disease (FILD). In some embodiments, the human subject has a blood relative with familial interstitial pneumonia (FIP). In some embodiments, the blood relative is a sibling. Alternatively, or in addition, in some embodiments, the human subject has a mutation in a sequence encoding MUC5B, TERC, FAM13A, TERT, DSP, AZGP1, OBFC1, ATP11A, IVD/DISP2, DPP9, SIGLEC14, ADM2, TSPAN5, CAMKK1 or MMP-7. In some embodiments, the mutation comprises a polymorphism in a sequence encoding a MUC5B promoter. In some embodiments, the polymorphism is rs35705950.

In some embodiments of the methods of treating a fibrotic lung disease in a human subject of the disclosure, the fibrotic lung disease is pulmonary fibrosis, idiopathic pulmonary fibrosis (IPF), an interstitial lung abnormality (ILA), or an asymptomatic ILA. In some embodiments, the fibrotic lung disease is pulmonary fibrosis or IPF. In some embodiments, the fibrotic lung disease is IPF.

In some embodiments of the methods of treating a fibrotic lung disease in a human subject of the disclosure, the method prevents the onset of a secondary condition associated with a severe form of the fibrotic lung disease. In some embodiments, the secondary condition comprises a collapsed lung, an infected lung, a blood clot in a lung, lung cancer, respiratory failure, pulmonary hypertension, heart failure or death.

### Idiopathic Pulmonary Fibrosis (IPF)

IPF is localized to the lung and is characterized by a pattern of heterogeneous, subpleural patches of fibrotic, remodeled lung, and often results in death within 3-5 years of diagnosis. IPF affects 5 million people worldwide, disproportionately affects men, is associated with cigarette smoking, increases with age, is inexplicably increasing in prevalence, and is likely underdiagnosed. Most patients with IPF are discovered in the advanced stage when little can be done to influence survival. There is a critical unmet need in idiopathic pulmonary fibrosis (IPF) for an early detection and prevention of IPF. Earlier diagnosis of IPF detects subjects with a lower burden of fibrotic lung disease providing an opportunity for secondary prevention of this progressive disease and changes the clinical approach to patients with IPF from palliative to preventive.

Early detection and prevention of idiopathic pulmonary fibrosis (IPF) is critical. As demonstrated herein, treatment of subjects at risk for developing PrePF is based on two central concepts of first, understanding that PrePF is essential for primary and secondary prevention of IPF and second, that similar to asymptomatic family members of familial IPF (FIP; ≥2 family members with IPF), asymptomatic family members of sporadic IPF represent an at-risk population for PrePF. These central concepts are supported by the observation that 1) IPF has a pre-symptomatic phase and PrePF appears to be a harbinger of IPF, 2) familial and sporadic IPF are similar etiologically, 3) MUC5B promoter variant is critical to early disease recognition and 4) identification of PrePF represents an opportunity to prevent extensive lung fibrosis. As shown herein, a common gain-of-function MUC5B promoter variant rs35705950 is a strong risk factor (genetic and otherwise), accounting for at least 30% of the total risk of developing IPF. The MUC5B promoter variant rs35705950 may be used to identify individuals with PrePF. MUC5B promoter variant rs35705950 is also predictive of radiographic progression of PrePF and is present in over 50% of non-Hispanic white patients with IPF and is also associated with unique clinical and biological IPF phenotypes. PrePF can be predicted using a combination of clinical risk factors, the MUC5B promoter variant rs35705950, and a panel of biomarkers. This disclosure provides methods of treating subjects with Preclinical Pulmonary Fibrosis (PrePF) and who may also be at risk for developing IPF. The methods of the disclosure fundamentally change the clinical approach to treating subjects with IPF, shifting the focus from a merely palliative to a proactive and preventive therapy.

### Rheumatoid Arthritis-Associated Interstitial Lung Disease (RA-ILD)

Rheumatoid arthritis (RA) is a common inflammatory and autoimmune disease that is associated with progressive impairment, systemic complications and increased mortality. Interstitial lung disease (RA-ILD) is detected in up to 60% of patients with RA on high-resolution computed-tomography (HRCT), is clinically significant in 10%, and is a leading cause of morbidity and mortality in patients with RA.

RA-ILD shares several characteristics with idiopathic pulmonary fibrosis (IPF), including common environmental risk factors, the high prevalence of the usual interstitial pneumonia (UIP) pattern, the progressive nature of the disease, and poor survival. The hypothesis of a shared genetic background between IPF and RA-ILD was recently suggested by a whole-exome sequencing (WES) genetic association study in patients with RA-ILD, revealing an excess of mutations in genes in RA-ILD previously associated with familial interstitial pneumonia (FIP) including TERT, RTEL1, PARN and SFTPC.

The common gain-of-function promoter variant rs3570595013 of the gene encoding mucin 5B (MUCSB) is the strongest genetic risk factor for IPF, observed in at least 50% of the cases of IPF and accounting for 30% of the risk of developing this disease. The MUCSB promoter variant is associated with increased expression of MUCSB in lung parenchyma of unaffected controls and cases of IPF. Consequently, it is hypothesized that the MUCSB promoter variant rs35705950 would also contribute to the occurrence of RA-ILD. To test this hypothesis, a multi-ethnic association study of the MUCSB promoter variant and RA-ILD in seven distinct case series was performed.

The MUC5B promoter variant rs35705950, the strongest genetic risk factor for IPF, is also a strong risk factor for RA-ILD, especially among those with radiographic evidence of UIP. Of note, the effect of the MUC5B promoter variant on the development of ILD associated with RA was similar in magnitude and direction to that observed in IPF.

The relationship between the MUC5B promoter variant and RA-ILD may be specific to UIP and may not be generalizable to other autoimmune conditions of the lung. The MUC5B promoter variant has not been found to be associated with risk of ILDs linked to systemic sclerosis or autoimmune myositis. Unlike these other types of ILD, RA-ILD shares more characteristics with IPF, notably the increased frequency of the UIP pattern (both radiologic and histologic), an increased prevalence of male sex and older age, and genetic susceptibility as assessed by an excess of mutations in genes linked to FIP in a cohort of RA-ILD, and now the MUC5B promoter variant rs35705950.

The disclosure demonstrates that the MUC5B promoter variant is a risk factor for UIP, and not simply limited to IPF and RA-ILD. In fact, emerging studies have identified the MUC5B promoter variant as a risk factor for chronic hypersensitivity pneumonitis, another condition known to have a sub-phenotype of UIP. Further, since HRCT underestimates the presence of ILD and the UIP pattern of fibrosis, our point estimates for association with the MUC5B variant are likely conservative. Similar to IPF, early forms of RA-ILD can be identified using the MUC5B promoter variant as biomarker.

The disclosure demonstrates that Muc5b is overexpressed by the bronchoalveolar epithelia and MUC5B mRNA is co-expressed by cells expressing surfactant protein C, as has been shown in IPF. These findings suggest either type 2 alveolar epithelial cells can express MUC5B or that in patients with RA-ILD, the cells in the distal airspace de-differentiate. Importantly, the disclosure demonstrates for the first time that cells that overexpress MUC5B are undergoing ER stress, a recognized mechanism of cell injury and repair. In aggregate, these findings indicate that the gain-of-function MUC5B promoter variant rs35705950 injures alveolar epithelia by inducing ER stress.

RA-ILD is a complex genetic phenotype with the minor allele of the MUC5B promoter variant rs35705950 identified as a risk factor for the disease. The odds ratios for the association of MUC5B promoter variant with RA-ILD is equivalent to that observed with IPF and substantively higher than those for the most other common risk variants for RA-ILD, including cigarette smoking and the human leukocyte antigen locus for RA.

The MUC5B promoter variant is a risk factor for UIP in general and may prove relevant beyond RA-ILD and IPF.

Expression of MUC5B in the bronchoalveolar epithelia co-incident with markers of ER stress suggest that the MUC5B promoter variant may be causing pulmonary fibrosis by initiating microscopic foci of injury and repair.

The MUC5B promoter variant appears to predict ILD in the RA population, identifying potential opportunities for early ILD detection in patients with RA.

### Preclinical Idiopathic Pulmonary Fibrosis

Better understanding and recognition of early pulmonary fibrosis is critical because medical therapies have been shown to slow progression, not to reverse or even stabilize established fibrosis-therefore, intervention before irreversible fibrosis has become extensive has the potential to improve quality of life and decrease morbidity. While IPF affects approximately 5 million people worldwide, between 1.8 and 14% of the general population ≥50 years of age have radiologic findings of undiagnosed pulmonary fibrosis. Large cohort studies indicate that interstitial lung abnormalities, postulated to represent early pulmonary fibrosis, are associated with increased mortality, and that most of these abnormalities progress over time. Members of families with 2 or more cases of pulmonary fibrosis (FIP, Familial Interstitial Pneumonia) have been identified as an “at-risk” population. In a previous study of FIP relatives, 14% had interstitial lung abnormalities on high resolution computed tomography (HRCT), and 35% had an abnormal transbronchial biopsy indicating interstitial lung disease.

HRCT provides visualization of the lung parenchyma and plays a key role in the diagnosis of the Idiopathic Interstitial Pneumonias (IIPs), including IPF. Currently, visual diagnosis by thoracic radiologists, in conjunction with multidisciplinary clinical conference, is the gold standard for diagnosing IIPs. However, visual assessment is imprecise and hampered by inter-observer variation. Quantitative HRCT (qHRCT) evaluation provides measures of fibrosis extent that, in subjects diagnosed with IPF, correlate with degree of physiologic impairment at baseline, and may be more sensitive to subtle changes in disease status than routinely used physiological metrics. The design and utility of quantitative methods in the context of early forms of fibrotic ILD requires further study. Deep learning methods have been increasingly used in imaging to identify and classify CT patterns, and may be particularly valuable in detection of early lung fibrosis.

PrePF is prevalent among FIP relatives, and a texture-based quantitative method of HRCT analyses is useful in identifying these abnormalities in this population, and key risk factors, including the MUC5B promoter variant, predict those at risk of this disease. PrePF subjects are older, more likely to be male, and more likely to have smoked than the unaffected subjects; additionally, the gain-of-function MUC5B promoter variant rs35705950, which has been shown in prior studies to be associated with pulmonary fibrosis, is more common in PrePF subjects when compared to their unaffected family members. Given the subtlety of the fibrotic change in many of these cases of PrePF, the high prevalence of potential UIP pattern on HRCT scan suggests that PrePF subjects may progress to IPF over time.

### Methods for Detecting a Genetic Variant

The present disclosure also provides methods of detecting the biomarkers of the present disclosure. Methods of detecting a genetic variant are further described in US Application US 2016-0060701A1 (the contents of which are incorporated herein by reference in their entirety). The practice of the present disclosure employs, unless otherwise indicated, conventional methods of analytical biochemistry, microbiology, molecular biology and recombinant DNA techniques within the skill of the art. Such techniques are explained fully in the literature. (See, e.g., Sambrook, J. et al. Molecular Cloning: A Laboratory Manual. 3rd, ed., Cold Spring Harbor Laboratory, Cold Spring Harbor Laboratory Press, Cold Spring Harbor, N. Y., 2000; DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M. Knipe, eds.)).

The methods of the invention are not limited to any particular way of detecting the presence or absence of a genetic variant (e.g. SNP) and can employ any suitable method to detect the presence or absence of a variant(s), of which numerous detection methods are known in the art. Dynamic allele-specific hybridization (DASH) can be used to detect a genetic variant. DASH genotyping takes advantage of the differences in the melting temperature in DNA that results from the instability of mismatched base pairs. The process can be vastly automated and encompasses a few simple principles. Thus, the aspects and embodiments described herein provide methods for assessing the presence or absence of SNPs in a sample (e.g. biological sample) from a subject suspected of having or developing an interstitial lung disease (e.g., because of family history). In certain embodiments, one or more SNPs are screened in one or more samples from a subject. The SNPs can be associated with one or more genes, e.g., one or more genes or other genes associated with mucous secretions as disclosed herein.

Typically, the target genomic segment is amplified and separated from non-target sequence, e.g., through use of a biotinylated primer and chromatography. A probe that is specific for the particular allele is added to the amplification product. The probe can be designed to hybridize specifically to a variant sequence or to the dominant allelic sequence. The probe can be either labeled with or added in the presence of a molecule that fluoresces when bound to double-stranded DNA. The signal intensity is then measured as temperature is increased until the Tm can be determined. A non-matching sequence (either genetic variant or dominant allelic sequence, depending on probe design), will result in a lower than expected Tm.

DASH genotyping relies on a quantifiable change in Tm, and is thus capable of measuring many types of mutations, not just SNPs. Other benefits of DASH include its ability to work with label free probes and its simple design and performance conditions.

Molecular beacons can also be used to detect a genetic variant. This method makes use of a specifically engineered single-stranded oligonucleotide probe. The oligonucleotide is designed such that there are complementary regions at each end and a probe sequence located in between. This design allows the probe to take on a hairpin, or stem-loop, structure in its natural, isolated state. Attached to one end of the probe is a fluorophore and to the other end a fluorescence quencher. Because of the stem-loop structure of the probe, the fluorophore is in close proximity to the quencher, thus preventing the molecule from emitting any fluorescence. The molecule is also engineered such that only the probe sequence is complementary to the targeted genomic DNA sequence.

If the probe sequence of the molecular beacon encounters its target genomic DNA sequence during the assay, it will anneal and hybridize. Because of the length of the probe sequence, the hairpin segment of the probe will be denatured in favor of forming a longer, more stable probe-target hybrid. This conformational change permits the fluorophore and quencher to be free of their tight proximity due to the hairpin association, allowing the molecule to fluoresce.

If on the other hand, the probe sequence encounters a target sequence with as little as one non-complementary nucleotide, the molecular beacon will preferentially stay in its natural hairpin state and no fluorescence will be observed, as the fluorophore remains quenched. The unique design of these molecular beacons allows for a simple diagnostic assay to identify SNPs at a given location. If a molecular beacon is designed to match a wild-type allele and another to match a mutant of the allele, the two can be used to identify the genotype of an individual. If only the first probe's fluorophore wavelength is detected during the assay then the individual is homozygous to the wild type. If only the second probe's wavelength is detected then the individual is homozygous to the mutant allele. Finally, if both wavelengths are detected, then both molecular beacons must be hybridizing to their complements and thus the individual must contain both alleles and be heterozygous.

A microarray can also be used to detect genetic variants. Hundreds of thousands of probes can be arrayed on a small chip, allowing for many genetic variants or SNPs to be interrogated simultaneously. Because SNP alleles only differ in one nucleotide and because it is difficult to achieve optimal hybridization conditions for all probes on the array, the target DNA has the potential to hybridize to mismatched probes. This can be addressed by using several redundant probes to interrogate each SNP. Probes can be designed to have the SNP site in several different locations as well as containing mismatches to the SNP allele. By comparing the differential amount of hybridization of the target DNA to each of these redundant probes, it is possible to determine specific homozygous and heterozygous alleles.

Restriction fragment length polymorphism (RFLP) can be used to detect genetic variants and SNPs. RFLP makes use of the many different restriction endonucleases and their high affinity to unique and specific restriction sites. By performing a digestion on a genomic sample and determining fragment lengths through a gel assay it is possible to ascertain whether or not the enzymes cut the expected restriction sites. A failure to cut the genomic sample results in an identifiably larger than expected fragment implying that there is a mutation at the point of the restriction site which is rendering it protected from nuclease activity.

PCR- and amplification-based methods can be used to detect genetic variants. For example, tetra-primer PCR employs two pairs of primers to amplify two alleles in one PCR reaction. The primers are designed such that the two primer pairs overlap at a SNP location but each matches perfectly to only one of the possible alleles. As a result, if a given allele is present in the PCR reaction, the primer pair specific to that allele will produce product but not the alternative allele with a different allelic sequence. The two primer pairs can be designed such that their PCR products are of a significantly different length allowing for easily distinguishable bands by gel electrophoresis, or such that they are differently labeled.

Primer extension can also be used to detect genetic variants. Primer extension first involves the hybridization of a probe to the bases immediately upstream of the SNP nucleotide followed by a ‘mini-sequencing’ reaction, in which DNA polymerase extends the hybridized primer by adding a base that is complementary to the SNP nucleotide. The incorporated base that is detected determines the presence or absence of the SNP allele. Because primer extension is based on the highly accurate DNA polymerase enzyme, the method is generally very reliable.

Primer extension is able to genotype most SNPs under very similar reaction conditions making it also highly flexible. The primer extension method is used in a number of assay formats, and can be detected using e.g., fluorescent labels or mass spectrometry.

Primer extension can involve incorporation of either fluorescently labeled ddNTP or fluorescently labeled deoxynucleotides (dNTP). With ddNTPs, probes hybridize to the target DNA immediately upstream of SNP nucleotide, and a single, ddNTP complementary to the SNP allele is added to the 3′ end of the probe (the missing 3′-hydroxyl in didioxynucleotide prevents further nucleotides from being added). Each ddNTP is labeled with a different fluorescent signal allowing for the detection of all four alleles in the same reaction. With dNTPs, allele-specific probes have 3′ bases which are complementary to each of the SNP alleles being interrogated. If the target DNA contains an allele complementary to the 3′ base of the probe, the target DNA will completely hybridize to the probe, allowing DNA polymerase to extend from the 3′ end of the probe. This is detected by the incorporation of the fluorescently labeled dNTPs onto the end of the probe. If the target DNA does not contain an allele complementary to the probe's 3′ base, the target DNA will produce a mismatch at the 3′ end of the probe and DNA polymerase will not be able to extend from the 3′ end of the probe.

The iPLEX® SNP genotyping method takes a slightly different approach, and relies on detection by mass spectrometer. Extension probes are designed in such a way that many different SNP assays can be amplified and analyzed in a PCR cocktail. The extension reaction uses ddNTPs as above, but the detection of the SNP allele is dependent on the actual mass of the extension product and not on a fluorescent molecule. This method is for low to medium high throughput, and is not intended for whole genome scanning.

Primer extension methods are, however, amenable to high throughput analysis. Primer extension probes can be arrayed on slides allowing for many SNPs to be genotyped at once. Broadly referred to as arrayed primer extension (APEX), this technology has several benefits over methods based on differential hybridization of probes. Comparatively, APEX methods have greater discriminating power than methods using differential hybridization, as it is often impossible to obtain the optimal hybridization conditions for the thousands of probes on DNA microarrays (usually this is addressed by having highly redundant probes).

Oligonucleotide ligation assays can also be used to detect genetic variants. DNA ligase catalyzes the ligation of the 3′ end of a DNA fragment to the 5′ end of a directly adjacent DNA fragment. This mechanism can be used to interrogate a SNP by hybridizing two probes directly over the SNP polymorphic site, whereby ligation can occur if the probes are identical to the target DNA. For example, two probes can be designed; an allele-specific probe which hybridizes to the target DNA so that its 3′ base is situated directly over the SNP nucleotide and a second probe that hybridizes the template upstream (downstream in the complementary strand) of the SNP polymorphic site providing a 5′ end for the ligation reaction. If the allele-specific probe matches the target DNA, it will fully hybridize to the target DNA and ligation can occur. Ligation does not generally occur in the presence of a mismatched 3′ base. Ligated or unligated products can be detected by gel electrophoresis, MALDI-TOF mass spectrometry or by capillary electrophoresis.

The 5′-nuclease activity of Taq DNA polymerase can be used for detecting genetic variants. The assay is performed concurrently with a PCR reaction and the results can be read in real-time. The assay requires forward and reverse PCR primers that will amplify a region that includes the SNP polymorphic site. Allele discrimination is achieved using FRET, and one or two allele-specific probes that hybridize to the SNP polymorphic site. The probes have a fluorophore linked to their 5′ end and a quencher molecule linked to their 3′ end. While the probe is intact, the quencher will remain in close proximity to the fluorophore, eliminating the fluorophore's signal. During the PCR amplification step, if the allele-specific probe is perfectly complementary to the SNP allele, it will bind to the target DNA strand and then get degraded by 5′-nuclease activity of the Taq polymerase as it extends the DNA from the PCR primers. The degradation of the probe results in the separation of the fluorophore from the quencher molecule, generating a detectable signal. If the allele-specific probe is not perfectly complementary, it will have lower melting temperature and not bind as efficiently. This prevents the nuclease from acting on the probe.

Forster resonance energy transfer (FRET) detection can be used for detection in primer extension and ligation reactions where the two labels are brought into close proximity to each other. It can also be used in the 5′-nuclease reaction, the molecular beacon reaction, and the invasive cleavage reactions where the neighboring donor/acceptor pair is separated by cleavage or disruption of the stem-loop structure that holds them together. FRET occurs when two conditions are met. First, the emission spectrum of the fluorescent donor dye must overlap with the excitation wavelength of the acceptor dye. Second, the two dyes must be in close proximity to each other because energy transfer drops off quickly with distance. The proximity requirement is what makes FRET a good detection method for a number of allelic discrimination mechanisms.

A variety of dyes can be used for FRET, and are known in the art. The most common ones are fluorescein, cyanine dyes (Cy3 to Cy7), rhodamine dyes (e.g. rhodamine 6G), the Alexa series of dyes (Alexa 405 to Alexa 730). Some of these dyes have been used in FRET networks (with multiple donors and acceptors). Optics for imaging all of these require detection from UV to near IR (e.g. Alex 405 to Cy7), and the Atto series of dyes (Atto-Tec GmbH). The Alexa series of dyes from Invitrogen cover the whole spectral range. They are very bright and photostable.

Example dye pairs for FRET labeling include Alexa-405/Alex-488, Alexa-488/Alexa-546, Alexa-532/Alexa-594, Alexa-594/Alexa-680, Alexa-594/Alexa-700, Alexa-700/Alexa-790, Cy3/Cy5, Cy3.5/Cy5.5, and Rhodamine-Green/Rhodamine-Red, etc. Fluorescent metal nanoparticles such as silver and gold nanoclusters can also be used (Richards et al. (2008) J Am Chem Soc 130:5038-39; Vosch et al. (2007) Proc Natl Acad Sci USA 104:12616-21; Petty and Dickson (2003) J Am Chem Soc 125:7780-81 Available filters, dichroics, multichroic mirrors and lasers can affect the choice of dye.

### In Vitro Complexes

Provided herein are nucleic acid complexes, e.g., formed in in vitro assays to indicate the presence of a genetic variant sequence. One of skill will understand that a nucleic acid complex can also be formed to detect the presence of a dominant allelic sequence, depending on the design of the probe or primer, e.g., in assays to distinguish homozygous and heterozygous subjects.

In some embodiments, the complex comprises a first nucleic acid hybridized to a genetic variant nucleic acid, wherein the genetic variant nucleic acid is a genetic variant in a gene selected from MUC5B, TERC, FAM13A, TERT, DSP, AZGP1, OBFC1, ATP11A, IVD/DISP2, DPP9, SIGLEC14, ADM2, TSPAN5, CAMKK1 or MMP-7. In some embodiments, the genetic variant nucleic acid is an amplification product. In some embodiments, the genetic variant nucleic acid is on genomic DNA, e.g., from a subject that has or is suspected of having an interstitial lung disease. In some embodiments, the first nucleic acid is an amplification product or a primer extension product. In some embodiments, the first nucleic acid is labeled. In some embodiments, the nucleic acid complex further comprises a second nucleic acid hybridized to the genetic variant nucleic acid. In some embodiments, the second nucleic acid is labeled e.g., with a FRET or other fluorescent label. In some embodiments, the first and second nucleic acids form a FRET pair when hybridized to a genetic variant sequence.

In some embodiments, the nucleic acid complex further comprises an enzyme, such as a DNA polymerase (e.g., standard DNA polymerase or thermostable polymerase such as Taq) or ligase.

The present disclosure includes but is not limited to the following embodiments:

A method for determining if an individual is predicted to develop and/or progress rapidly with an interstitial pneumonia comprising: detecting in a biological sample from the individual, at least one of: a) the presence of a marker polymorphism selected from the group consisting of: rs35705950; and/or, b) a level of gene expression of a marker gene or plurality of marker genes selected from the group consisting of: a marker gene having at least 95% sequence identity with at least one sequence selected from the group consisting of MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, or homologs or variants thereof; c) polypeptides encoded by the marker genes of b) d) fragments of polypeptides of c); and e) a polynucleotide which is fully complementary to at least a portion of a marker gene of b); wherein the presence of the plurality of markers is indicative of whether an individual will develop a disease. In some embodiments, the genes detected share 100% sequence identity with the corresponding marker gene in b). In some embodiments, the presence or level of at least one of the plurality of markers is determined and compared to a standard level or reference set. In some embodiments, the standard level or reference set is determined according to a statistical procedure for risk prediction. In some embodiments, the statistical procedure for risk prediction comprises using the sum of the gene expression of the marker or markers or the presence or absence of a set of markers, weighted by a Proportional Hazards coefficient. In some embodiments, the presence of the at least one marker is determined by detecting the presence or absence or expression level of a polypeptide. In some embodiments, the method further comprises detecting the presence of the polypeptide using a reagent that specifically binds to the polypeptide or a fragment thereof. In some embodiments, the reagent is selected from the group consisting of an antibody, an antibody derivative, and an antibody fragment. In some embodiments, the presence of the marker is determined by obtaining the sequence of genomic DNA at the locus of the polymorphism. In some embodiments, the presence of the marker is determined by obtaining RNA from the biological sample; generating cDNA from the RNA; amplifying the cDNA with probes or primers for marker genes; obtaining from the amplified cDNA the expression levels of the genes or gene expression products in the sample. In some embodiments, the individual is a human.

In some embodiments, the method further comprises: a) comparing the expression level of the marker gene or plurality of marker genes in the biological sample to a control level of the marker gene(s) selected from the group consisting of: a control level of the marker gene that has been correlated with interstitial lung disease, the risk of developing interstitial lung disease, or having a interstitial lung disease; and a control level of the marker that has been correlated with slow or no progression of interstitial lung disease, or low risk of developing an interstitial lung disease; and b) selecting the individual as being predicted to progress rapidly in the development of interstitial pneumonia, if the expression level of the marker gene in the individual's biological sample is statistically similar to, or greater than, the control level of expression of the marker gene that has been correlated with interstitial lung disease, or c) selecting the individual as being predicted to not develop interstitial lung disease, or to progress slowly, if the level of the marker gene in the individual's biological sample is statistically less than the control level of the marker gene that has been correlated with interstitial lung disease.

In some embodiments, the method further comparing the presence of a polymorphism, in the biological sample to a set of genetic variants or polymorphic markers from an individual or control group having developed interstitial lung disease, and, selecting the individual as being predicted to develop or to progress with interstitial pneumonia if the polymorphic markers present in the biological sample are identical to or statistically similar to a set of polymorphic markers from the individual or control group or, selecting the individual as being predicted to develop or rapidly progress with interstitial pneumonia, if the polymorphic markers present in the biological sample are not identical to or statistically similar to the set of genetic variants or polymorphic markers from the individual or control group.

A method for monitoring the progression of interstitial lung disease in a subject, comprising: i) measuring expression levels of a plurality of gene markers in a first biological sample obtained from the subject, wherein the plurality of markers comprise a plurality of markers selected from the group consisting of: a marker gene having at least 95% sequence identity with a sequence selected from the group consisting of a) MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, or homologs or variants thereof, b) polypeptides encoded by the marker genes of a), c) fragments of polypeptides of d); and e) a polynucleotide which is fully complementary to at least a portion of a marker gene of b); ii) measuring expression levels of the plurality of markers in a second biological sample obtained from the subject; and iii) comparing the expression level of the marker measured in the first sample with the level of the marker measured in the second sample. In some embodiments, the marker genes detected share 100% sequence identity with the corresponding marker gene in a). In some embodiments, the method further comprises performing a follow-up step selected from the group consisting of CT scan of the chest and pathological examination of lung tissues from the subject. In some embodiments, the first biological sample from the subject is obtained at a time to, and the second biological sample from the subject is obtained at a later time ti. In some embodiments, the first biological sample and the second biological sample are obtained from the subject are obtained more than once over a range of times.

A method of assessing the efficacy of a treatment for interstitial lung disease or interstitial pneumonia in a subject, the method comprising comparing: i) the expression level of a marker measured in a first sample obtained from the subject at a time to, wherein the marker is selected from the group consisting of a) a marker gene having at least 95% sequence identity with a sequence selected from the group consisting of MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, or homologs or variants thereof, b) polypeptides encoded by the marker genes of a) c) fragments of polypeptides of b); and d) a polynucleotide which is fully complementary to at least a portion of a marker gene of a); ii) the level of the marker in a second sample obtained from the subject at time ti; and, iii) performing a follow-up step selected from CT scan of the chest and pathological examination of lung tissues from the subject; wherein a decrease in the level of the marker in the second sample relative to the first sample is an indication that the treatment is efficacious for treating interstitial pneumonia in the subject. In some embodiments, the genes detected share 100% sequence identity with the corresponding marker gene in a). In some embodiments, the time t0 is before the treatment has been administered to the subject, and the time t1 is after the treatment has been administered to the subject. In some embodiments, the comparing is repeated over a range of times.

An assay system for predicting individual prognosis therapy for interstitial pneumonia comprising a means to detect at least one of: a) the presence of a marker polymorphism selected from the group consisting of: rs35705950; and/or, b) a level of gene expression of a marker gene or plurality of marker genes selected from the group consisting of: a marker gene having at least 95% sequence identity with a sequence selected from the group consisting of MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, or homologs or variants thereof; c) polypeptides encoded by the marker genes of b) d) fragments of polypeptides of c); and e) a polynucleotide which is fully complementary to at least a portion of a marker gene of b). In some embodiments, the means to detect comprises nucleic acid probes comprising at least 10 to 50 contiguous nucleic acids of the marker polymorphisms or gene(s), or complementary nucleic acid sequences thereof. In some embodiments, the means to detect comprises binding ligands that specifically detect polypeptides encoded by the marker genes. In some embodiments, the genes detected share 100% sequence identity with the corresponding marker gene in b). In some embodiments, the means to detect comprises at least one of nucleic acid probe and binding ligands disposed on an assay surface. In some embodiments, the assay surface comprises a chip, array, or fluidity card. In some embodiments, the probes comprise complementary nucleic acid sequences to at least 10 to 50 nucleic acid sequences of the marker genes. In some embodiments, the binding ligands comprise antibodies or binding fragments thereof. In some embodiments, the assay system further comprises: a control selected from information containing a predetermined control level or set of genetic variants or polymorphic markers that has been correlated with diagnosis, development, progression, or life expectancy in interstitial lung disease patients.

A method of detecting a level of gene expression of one or more marker genes in a human subject with interstitial pneumonia, comprising, optionally, obtaining a biological sample from a human individual with interstitial pneumonia; detecting the level of expression of a gene selected from MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, or homologs or variants thereof, in one or more cells from the biological sample from the individual. In some embodiments, the method further comprises detecting the level of expression of a gene selected from MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, or homologs or variants thereof, in one or more cells from the biological sample from the individual. In some embodiments, the method further comprises detecting the level of expression of a gene selected from MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, or homologs or variants thereof in one or more cells from the biological sample from the individual.

A method of treating an interstitial lung disease in a subject in need of such treatment, comprising: detecting a level of one or more marker genes selected from MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, or homologs or variants thereof in a biological sample obtained from the human subject; and, administering an effective amount of an effective treatment. In some embodiments, the method further comprises detecting the level of expression of a gene selected from MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, or homologs or variants thereof, in one or more cells from the biological sample from the individual. In some embodiments, the method further comprises detecting the level of expression of a gene selected from MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, or homologs or variants thereof, in one or more cells from the biological sample from the individual.

### Detection of Genetic Variants

Methods of detecting a genetic variant are further described, for example, in U.S. Pat. No. 8,673,565 (the contents of which are herein incorporated by reference in their entirety). Genetic variations in the mucin genes are associated with pulmonary diseases. These genetic variations can be found in any part of the gene, e.g., in the regulatory regions, introns, or exons. Relevant genetic variations may also be found the intergene regions, e.g., in sequences between mucin genes. Insertions, substitutions, and deletions are included in genetic variants. Single nucleotide polymorphisms (SNPs) are exemplary genetic variants.

In particular, 14 independent SNPs are associated with pulmonary disorders (e.g. FIP or IPF). The studies disclosed herein demonstrate that presence of one or more of these SNPs associated with MUC5B can lead to predisposition to a pulmonary disorder. In addition, in some embodiments, if present, some of these SNPs are related to a transcription factor binding site. The transcription factor binding site can effect modulation of MUC5B expression, for example E2F3 loss, and HOXA9 and PAX-2 generation.

The disclosure thus provides methods for assessing the presence or absence of SNPs in a sample from a subject suspected of having or developing a pulmonary disorder (e.g., because of family history). In certain embodiments, one or more SNPs are screened in one or more samples from a subject. The SNPs can be associated with one or more genes, e.g., one or more MUC genes or other genes associated with mucous secretion. In some embodiments, a MUC gene associated SNP is associated with MUC5B and/or another MUC gene, such as MUC5AC or MUC1. SNPs contemplated for diagnostic, treatment, or prognosis can include SNPs found within a MUC gene and/or within a regulatory or promoter region associated with a MUC gene. For example, one or more SNPs can include, but are not limited to, detection of the SNPs of MUC5B alone or in combination with other genetic variations or SNPs and/or other diagnostic or prognostic methods.

Methods for detecting genetic variants such as a SNP are known in the art, e.g., Southern or Northern blot, nucleotide array, amplification methods, etc. Primers or probes are designed to hybridize to a target sequence. For example, genomic DNA can be screened for the presence of an identified genetic element of using a probe based upon one or more sequences, e.g., using a probe with substantial identity to a subsequence of the MUC5B gene. Expressed RNA can also be screened, but may not include all relevant genetic variations. Various degrees of stringency of hybridization may be employed in the assay. As the conditions for hybridization become more stringent, there must be a greater degree of complementarity between the probe and the target for duplex formation to occur. Thus, high stringency conditions are typically used for detecting a SNP.

Thus, in some embodiments, a genetic variant MUC5B gene in a subject is detected by contacting a nucleic acid in a sample from the subject with a probe having substantial identity to a subsequence of the MUC5B gene, and determining whether the nucleic acid indicates that the subject has a genetic variant MUC5B gene. In some cases, the sample can be processed prior to amplification, e.g., to separate genomic DNA from other sample components. In some cases, the probe has at least 90, 92, 94, 95, 96, 98, 99, or 100% identity to the MUC5B gene subsequence. Typically, the probe is between 10-500 nucleotides in length, e.g., 10-100, 10-40, 10-20, 20-100, 100-400, etc. In the case of detecting a SNP, the probe can be even shorter, e.g., 8-20 nucleotides in length. In some cases, the MUC5B gene sequence to be detected includes at least 8 contiguous nucleotides, e.g., at least 10, 15, 20, 25, 30, 35 or more contiguous nucleotides. In some embodiments, the sequence to be detected includes 8 contiguous nucleotides, e.g., at least 10, 15, 20, 25, 30, 35 or more contiguous nucleotides.

The degree of stringency can be controlled by temperature, ionic strength, pH and/or the presence of a partially denaturing solvent such as formamide. For example, the stringency of hybridization is conveniently varied by changing the concentration of formamide within the range up to and about 50%. The degree of complementarity (sequence identity) required for detectable binding will vary in accordance with the stringency of the hybridization medium and/or wash medium. In certain embodiments, in particular for detection of a particular SNP, the degree of complementarity is about 100 percent. In other embodiments, sequence variations can result in <100% complementarity, <90% complementarity probes, <80% complementarity probes, etc., in particular, in a sequence that does not involve a SNP. In some examples, e.g., detection of species homologs, primers may be compensated for by reducing the stringency of the hybridization and/or wash medium.

High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. Other exemplary conditions are disclosed in the following Examples. It is understood that the temperature and ionic strength of a desired stringency are determined in part by the length of the particular nucleic acid(s), the length and nucleotide content of the target sequence(s), the charge composition of the nucleic acid(s), and by the presence or concentration of formamide, tetramethylammonium chloride or other solvent(s) in a hybridization mixture. Nucleic acids can be completely complementary to a target sequence or exhibit one or more mismatches.

Nucleic acids of interest can also be amplified using a variety of known amplification techniques. For instance, polymerase chain reaction (PCR) technology may be used to amplify target sequences (e.g., genetic variants) directly from DNA, RNA, or cDNA. In some embodiments, a stretch of nucleic acids is amplified using primers on either side of a targeted genetic variation, and the amplification product is then sequenced to detect the targeted genetic variation (using, e.g., Sanger sequencing, Pyrosequencing, Nextgen® sequencing technologies). For example, the primers can be designed to hybridize to either side of the upstream regulatory region of the MUC5B gene, and the intervening sequence determined to detect a SNP in the promoter region. In some embodiments, one of the primers can be designed to hybridize to the targeted genetic variant. In some cases, a genetic variant nucleotide can be identified using RT-PCR, e.g., using labeled nucleotide monomers. In this way, the identity of the nucleotide at a given position can be detected as it is added to the polymerizing nucleic acid. The Scorpion™ system is a commercially available example of this technology.

Thus, in some embodiments, a genetic variant MUC5B gene in a subject is detected by amplifying a nucleic acid in a sample from the subject to form an amplification product, and determining whether the amplification product indicates a genetic variant MUC5B gene. In some cases, the sample can be processed prior to amplification, e.g., to separate genomic DNA from other sample components. In some cases, amplifying comprises contacting the sample with amplification primers having substantial identity to MUC5B genomic subsequences, e.g., at least 90, 92, 94, 95, 96, 98, 99, or 100% identity. Typically, the sequence to be amplified is between 30-1000 nucleotides in length, e.g., 50-500, 50-400, 100-400, 50-200, 100-300, etc. In some cases, the sequence to be amplified or detected includes at least 8 contiguous nucleotides, e.g., at least 10, 15, 20, 25, 30, 35 or more contiguous nucleotides. In some embodiments, the sequence to be amplified or detected includes 8 contiguous nucleotides, e.g., at least 10, 15, 20, 25, 30, 35 or more contiguous nucleotides. In some aspects, the contiguous nucleotides include nucleotide 28.

Amplification techniques can also be useful for cloning nucleic acid sequences, to make nucleic acids to use as probes for detecting the presence of a target nucleic acid in samples, for nucleic acid sequencing, for control samples, or for other purposes. Probes and primers are also readily available from commercial sources, e.g., from Invitrogen, Clonetech, etc.

### Detection of Expression Levels

Expression of a given gene, e.g., MUC5B or another mucin, pulmonary disease marker, or standard (control), is typically detected by detecting the amount of RNA (e.g., mRNA) or protein. Sample levels can be compared to a control level.

Methods for detecting RNA are largely cumulative with the nucleic acid detection assays described above. RNA to be detected can include mRNA. In some embodiments, a reverse transcriptase reaction is carried out and the targeted sequence is then amplified using standard PCR. Quantitative PCR (qPCR) or real time PCR (RT-PCR) is useful for determining relative expression levels, when compared to a control. Quantitative PCR techniques and platforms are known in the art, and commercially available (see, e.g., the qPCR Symposium website, available at qpersymposium.com). Nucleic acid arrays are also useful for detecting nucleic acid expression. Customizable arrays are available from, e.g., Affimatrix. An exemplary human MUC5B mRNA sequence, e.g., for probe and primer design, can be found at GenBank Accession No. AF086604.1.

Protein levels can be detected using antibodies or antibody fragments specific for that protein, natural ligands, small molecules, aptamers, etc. An exemplary human MUC5B sequence, e.g., for screening a targeting agent, can be found at UniProt Accession No. 000446.

Antibody based techniques are known in the art, and described, e.g., in Harlow & Lane (1988) Antibodies: A Laboratory Manual and Harlow (1998) Using Antibodies: A Laboratory Manual; Wild, The Immunoassay Handbook, 3d edition (2005) and Law, Immunoassay: A Practical Guide (1996). The assay can be directed to detection of a molecular target (e.g., protein or antigen), or a cell, tissue, biological sample, liquid sample or surface suspected of carrying an antibody or antibody target.

A non-exhaustive list of immunoassays includes: competitive and non-competitive formats, enzyme linked immunosorption assays (ELISA), microspot assays, Western blots, gel filtration and chromatography, immunochromatography, immunohistochemistry, flow cytometry or fluorescence activated cell sorting (FACS), microarrays, and more. Such techniques can also be used in situ, ex vivo, or in vivo, e.g., for diagnostic imaging.

Aptamers are nucleic acids that are designed to bind to a wide variety of targets in a non-Watson Crick manner. An aptamer can thus be used to detect or otherwise target nearly any molecule of interest, including a pulmonary disease associated protein. Methods of constructing and determining the binding characteristics of aptamers are well known in the art. For example, such techniques are described in U.S. Pat. Nos. 5,582,981, 5,595,877 and 5,637,459. Aptamers are typically at least 5 nucleotides, 10, 20, 30 or 40 nucleotides in length, and can be composed of modified nucleic acids to improve stability. Flanking sequences can be added for structural stability, e.g., to form 3-dimensional structures in the aptamer.

Protein detection agents described herein can also be used as a treatment and/or diagnosis of pulmonary disease or predictor of disease progression, e.g., propensity for survival, in a subject having or suspected of developing a pulmonary disorder. In certain embodiments, MUC5B antibodies can be used to assess MUC5B protein levels in a subject having or suspected of developing a pulmonary disorder. It is contemplated herein that antibodies or antibody fragments may be used to modulate MUC5B production in a subject having or suspected of developing a pulmonary disease. In certain embodiments, one or more agents capable of modulating MUC5B may be used to treat a subject having or suspected of developing a pulmonary disorder. One or more antibodies or antibody fragments may be generated to detect one or more of the SNPs disclosed herein by any method known in the art.

In certain embodiments, MUC5B diagnostic tests may include, but are not limited to, alone or in combination, analysis of rs35705950 SNP in MUC5B gene, MUC5B mRNA levels, and/or MUC5B protein levels.

### Additional Pulmonary Disease Markers

The above methods of detection can be applied to additional pulmonary disease markers. That is, the expression level or presence of genetic variants of at least one additional pulmonary disease marker gene can be determined, or the activity of the marker protein can be determined, and compared to a standard control for the pulmonary disease marker. The examination of additional pulmonary disease markers can be used to confirm a diagnosis of pulmonary disease, monitor disease progression, or determine the efficacy of a course of treatment in a subject.

In some cases, pulmonary disease is indicated by an increased number of lymphocytes, e.g., CD4+CD28− cells.

Genetic variations in the following genes are associated with pulmonary disease: Surfactant Protein A2, Surfactant Protein B, Surfactant Protein C, TERC, TERT, IL-1RN, IL-1α, IL-1β, TNF, Lymphotoxin α, TNF-RII, IL-10, IL-6, IL-12, IFNγ, TGFβ, CR1, ACE, IL-8, CXCR1, CXCR2, MUC1 (KL6), or MUC5AC. Thus, the invention further includes methods of determining whether the genome of a subject comprises a genetic variant of at least one gene selected from these genes. The presence of a genetic variant indicates that the subject has or is at risk of developing pulmonary disease. Said determining can optionally be combined with determining whether the genome of the subject comprises a genetic variant MUC5B gene, or determining whether the subject has an elevated level of MUC5B RNA or protein to confirm or strengthen the diagnosis or prognosis.

Abnormal expression in the following genes can also be indicative of pulmonary disease: Surfactant Protein A, Surfactant Protein D, KL-6/MUC1, CC16, CK-19, Ca 19-9, SLX, MCP-1, MIP-1a, ITAC, glutathione, type III procollagen peptide, sIL-2R, ACE, neopterin, beta-glucuronidase, LDH, CCL-18, CCL-2, CXCL12, MMP7, and osteopontin. Thus, the expression of one of these genes can be detected and compared to a control, wherein an abnormal expression level indicates that the subject has or is at risk of developing pulmonary disease. Said determining can optionally be combined with determining whether the genome of the subject comprises a genetic variant MUC5B gene, or determining whether the subject has an elevated level of MUC5B RNA or protein to confirm or strengthen the diagnosis or prognosis.

### Biomarkers

The present disclosure provides a peripheral blood biomarker profile for IPF to demonstrate the use of a predictive biomarker profile in cases of preclinical pulmonary fibrosis (PrePF) derived from families with familial IPF. The present disclosure also provides biomarker identification for association between each genetic, epigenetic or protein (gene product) biomarker with PrePF and the predictive value of the combination of biomarkers associated with PrePF.

A large cohort of families with familial IPF for genetic research was established, including 937 families with ≥2 cases of IPF, and 2375 family members that have been previously phenotyped as unaffected. This study focuses on subjects with PrePF to elucidate the processes active in early disease pathogenesis and to predict or prevent the irreversible fibroproliferative process. Genetic risk factors, especially the MUC5B promoter variant, identifies individuals with preclinical interstitial changes on chest CT scan that progress and are associated with reduced survival. Biomarkers may be used to identify those subjects with PrePF among those at-risk for IPF. Given the irreversible nature of IPF, even approved treatments (pirfenidone and nintedanib) only modestly slow progression and have not been shown to alter the 3-5 year survival. Pirfenidone and nintedanib are effective in patients with mild disease, suggesting that patients with PrePF may be targeted for early intervention, before most of the lung has been irreversibly remodeled.

Table 1 below shows additional gene expression changes present in subjects with IPF compared to controls. Specifically, the expression of the genes listed in Table 1 are upregulated in IPF compared to the expression of these same genes in control subjects. Accordingly, the discovery of elevated expression levels of one or more genes listed in Table 1 compared to a control in an asymptomatic subject may indicate that the subject has PrePF and/or that the subject is at risk for developing IPF.

In some embodiments of the methods of the disclosure, the subject has a mutation in a nucleic acid or amino acid sequence encoding a gene or gene product that is upregulated in a subject having a fibrotic pulmonary disease of the disclosure. In some embodiments of the methods of the disclosure, the subject has a mutation in a nucleic acid or amino acid sequence encoding Leukotriene A4 Hydrolase (LTA4H), Surfactant Protein B (SFTPB), Breast Cancer Anti-Estrogen Resistance 3 (BCAR3), C—X—C motif Chemokine Ligand 13 (CXCL13), EPH Receptor A2 (EPHA2), Serum Amyloid A1 (SAA1), Phospholipase A2 Group IIA (PLA2G2A), Insulin-Like Growth Factor Binding Protein 3 (IGFBP3), C—C Motif Chemokine Ligand 28 (CCL28), S100 Calcium Binding Protein A12 (S100A12), Thromboxane A Synthase 1 (TBXAS1), Leukocyte Cell Derived Chemotaxin 1 (LECT1), Complement C3 (C3), Gastrin Releasing Peptide (GRP), C-Reactive Protein (CRP), Vitrin (VIT), Insulin-Like Growth Factor Binding Protein 1 (IGFBP1), Family with Sequence Similarity 173 Member A (FAM173A), Natriuretic Peptide A (NPPA), Secreted Frizzled Related Protein 1 (SFRP1), Ezrin (EZR), Inter-Alpha-Trypsin Inhibitor Heavy Chain Family Member 5 (ITIH5), Pleckstrin and Sec7 Domain Containing 2 (PSD2), Galectin 3 Binding Protein (LGALS3BP), Catenin Beta 1 (CTNNB1), Chromodomain Y Like 2 (CDYL2), Matrix Metallopeptidase 7 (MMP7), Apolipoprotein B (APOB), Proline and Arginine Rich End Leucine Rich Repeat Protein (PRELP), Eukaryotic Translation Initiation Factor 1A, X-linked (EIF1AX), Mesencephalic Astrocyte Derived Neurotrophic Factor (MANF), TNF Receptor Superfamily Member 13C (TNFRSF13C), Deformed Epidermal Autoregulatory Factor 1 transcription factor (DEAF1), Tumor Protein Translationally-Controlled 1 (TPT1), Unc-5 Netrin Receptor B (UNC5B), Phosphatidylethanolamine Binding Protein 1 (PEBP1), Syntaxin 8 (STX8), Polymeric Immunoglobulin Receptor (PIGR), Adenine Phosphoribosyltransferase (APRT), Matrix Metallopeptidase 3 (MMP3), Galectin 7 (LGALS7), Bruton Tyrosine Kinase (BTK), NSFL1 Cofactor (NSFL1C), FER Tyrosine Kinase (FER), Regenerating Family Member 1 Beta (REG1B), SMAD Family Member 2 (SMAD2), Interleukin 1 Receptor Like 1 (IL1RL1), C—C Motif Chemokine Ligand 18 (CCL18), Acid Phosphatase 2 Lysosomal (ACP2), Eukaryotic Translation Initiation Factor 4E Family Member 2 (EIF4E2), Neurexin 3 (NRXN3), IGF Like Family Member 1 (IGFL1), NME/NM23 Nucleoside Diphosphate Kinase 1 (NME1), Potassium Voltage-Gated Channel Isk-Related Family Member 1-Like (KCNE1L) or Neurexophilin 2 (NXPH2).

TABLE 1

| TARGET_GENE_SYM- | ORGAN- | | B—H | Fold |
|---|---|---|---|---|
| BOL | ISM | p-value | q-value | Change |
| LTA4H | Human | 8.70E−43 | 3.13E−39 | 3.912 |
| SFTP8 | Human | 1.17E−37 | 2.10E−34 | 3.399 |
| BCAR3 | Human | 4.28E−25 | 3.85E−22 | 2.906 |
| CXCL13 | Human | 1.30E−29 | 1.56E−26 | 2.904 |
| EPHA2 | Human | 9.62E−23 | 6.93E−20 | 2.651 |
| SAA1 | Human | 6.01E−07 | 7.84E−06 | 2.631 |
| PLA2G2A | Human | 8.19E−21 | 2.95E−18 | 2.171 |
| Igfbp3 | Mouse | 1.18E−18 | 2.66E−16 | 2.149 |
| CCL28 | Human | 1.22E−22 | 7.30E−20 | 2.135 |
| S100A12 | Human | 1.06E−20 | 3.45E−18 | 2.125 |
| TBXAS1 | Human | 1.60E−21 | 7.20E−19 | 2.11 |
| LECT1 | Human | 4.17E−19 | 1.00E−16 | 2.082 |
| C3 | Human | 7.08E−07 | 8.95E−06 | 2.062 |
| GRP | Human | 8.35E−09 | 1.66E−07 | 1.988 |
| CSP | Human | 1.36E−08 | 2.61E−07 | 1.957 |
| VIT | Human | 2.47E−17 | 4.45E−15 | 1.929 |
| IGFBP1 | Human | 4.32E−11 | 1.56E−09 | 1.914 |
| FAM173A | Human | 2.19E−13 | 1.84E−11 | 1.904 |
| NPPA | Human | 5.02E−12 | 2.58E−10 | 1.877 |
| SFRP1 | Human | 1.74E−20 | 5.23E−18 | 1.866 |
| EZR | Human | 6.41E−10 | 1.72E−08 | 1.809 |
| ITIH5 | Human | 5.11E−21 | 2.04E−18 | 1.705 |
| PSD2 | Human | 5.38E−18 | 1.08E−15 | 1.689 |
| LGALS38P | Human | 8.06E−22 | 4.15E−19 | 1.678 |
| | | 1.18E−05 | 0.000102 | 1.668 |
| CTNNB1 | Human | 5.66E−12 | 2.87E−10 | 1.625 |
| CDYL2 | Human | 4.11E−07 | 5.59E−06 | 1.622 |
| MMP7 | Human | 1.56E−19 | 4.02E−17 | 1.621 |
| APOB | Human | 8.73E−13 | 6.42E−11 | 1.597 |
| PRELP | Human | 1.13E−10 | 3.53E−09 | 1.595 |
| EIF1AX | Human | 2.13E−06 | 2.31E−05 | 1.59 |
| MANF | Human | 0.00458 | 0.015006 | 1.585 |
| TNFRSF13C | Human | 1.77E−11 | 7.31E−10 | 1.573 |
| C3 | Human | 2.40E−16 | 3.93E−14 | 1.566 |
| DEAF1 | Human | 0.000221 | 0.001192 | 1.565 |
| TPT1 | Human | 1.22E−12 | 7.82E−11 | 1.548 |
| UNC5B | Human | 2.06E−34 | 2.18E−12 | 1.547 |
| PEBP1 | Human | 4.92E−11 | 1.72E−09 | 1.544 |
| STX8 | Human | 8.82E−12 | 4.13E−10 | 1.537 |
| PIGR | Human | 1.29E−09 | 3.19E−08 | 1.532 |
| APRT | Human | 1.51E−07 | 2.26E−06 | 1.525 |
| MMP3 | Human | 9.50E−07 | 1.15E−05 | 1.524 |
| LGALS7 | Human | 7.51E−05 | 0.000474 | 1.514 |
| BTK | Human | 1.47E−09 | 3.52E−08 | 1.511 |
| NSFL1C | Human | 7.33E−11 | 2.40E−09 | 1.506 |
| FER | Human | 2.24E−07 | 3.24E−06 | 1.503 |
| REG1B | Human | 6.68E−11 | 2.25E−09 | 1.502 |
| SMAD2 | Human | 4.39E−10 | 1.25E−08 | 1.493 |
| IL1RL1 | Human | 9.55E−07 | 1.15E−05 | 1.492 |
| CCL18 | Human | 1.25E−13 | 1.07E−11 | 1.491 |
| ACP2 | Human | 3.73E−08 | 6.33E−07 | 1.488 |
| EIF4E2 | Human | 1.67E−12 | 1.02E−10 | 1.483 |
| NRXN3 | Human | 2.33E−17 | 4.42E−15 | 1.48 |
| IGFL1 | Human | 5.07E−10 | 1.40E−08 | 1.474 |
| NME1 | Human | 1.43E−10 | 4.39E−09 | 1.463 |
| KCNE1L | Human | 3.93E−20 | 1.09E−17 | 1.462 |
| NXPH2 | Human | 9.66E−30 | 2.47E−08 | 1.451 |

Table 2 below shows additional gene expression changes present in subjects with IPF compared to controls. Specifically, the expression of the genes listed in Table 2 are downregulated in IPF compared to the expression of these same genes in control subjects. Accordingly, the discovery of decreased expression levels of one or more genes listed in Table 2 compared to a control in an asymptomatic subject may indicate that the subject has PrePF and/or that the subject is at risk for developing IPF.

In some embodiments of the methods of the disclosure, the subject has a mutation in a nucleic acid or amino acid sequence encoding a gene or gene product that is downregulated in a subject having a fibrotic pulmonary disease of the disclosure. In some embodiments of the methods of the disclosure, the subject has a mutation in a nucleic acid or amino acid sequence encoding Surfactant Protein D (SFTPD), Glyceraldehyde-3-Phosphate Dehydrogenase (GAPDH), Histone Cluster 1 H1 Family Member C (HIST1H1C), YTH Domain Containing 1 (YTHDC1), Plexin A1 (PLXNA1), Serine Peptidase Inhibitor Kazal Type 6 (SPINK6), LDL Receptor Related Protein Associated Protein 1 (LRPAP1), Secretoglobin Family 3A Member 1 (SCGB3A1), H2A Histone Family Member Z (H2AFZ) or Chromosome 1 Open Reading Frame 162 (C1orf162).

TABLE 2

| TARGET_GENE_SYM- | ORGAN- | | B—H | Fold |
|---|---|---|---|---|
| BOL | ISM | p-value | q-value | Change |
| SFTPD | Human | 8.19E−15 | 9.83E−13 | −2.262 |
| GAPDH | Human | 1.46E−09 | 3.52E−08 | −2.096 |
| HIST1H1C | Human | 3.68E−18 | 7.80E−16 | −2.011 |
| | | 3.63E−16 | 5.69E−14 | −1.964 |
| YTHDC1 | Human | 1.19E−11 | 5.38E−10 | −1.699 |
| PLXNA1 | Human | 1.64E−12 | 1.02E−10 | −1.64 |
| SPINK6 | Human | 3.68E−07 | 5.04E−06 | −1.635 |
| LRPAP1 | Human | 2.65E−15 | 3.53E−13 | −1.521 |
| SCGB3A1 | Human | 3.35E−07 | 4.61E−06 | −1.518 |
| H2AFZ | Human | 3.91E−14 | 3.91E−12 | −1.501 |
| | | 2.95E−11 | 1.16E−09 | −1.493 |
| C1orf162 | Human | 1.29E−84 | 7.52E−04 | −1.458 |

In some embodiments of the methods of the disclosure, the subject having PrePF or at risk of developing IPF has a mutation in a sequence encoding MUC5B, TERC, FAM13A, TERT, DSP, AZGP1, OBFC1, ATP11A, IVD/DISP2, DPP9, SIGLEC14, ADM2, TSPAN5, CAMKK1 or MMP-7.

In some embodiments of the methods of the disclosure, the subject having PrePF or at risk of developing IPF has a mutation in a sequence encoding Telomerase RNA Component (TERC). In some embodiments the polymorphism is rs6793295 comprising (SEQ ID NO: 1).

(SEQ ID NO: 1)
AGAAAGAAGT CATGAAAGTA GGAACCACAT
TTTTACTCAT CTTTCTGTCT CCAGCAAGCA
GCTTACTGCT TTTCATACAC ATTTTGCTTT
TATTACTCAT GATTTCAAAG GTGTAATGGT
TCAGCCACAT CAATGTAACA AACAGTTCAC
ACTGGGCTCT TATAGTCTGG CCTTTAAAAC
CTTCACTATT TATGCTTTCA TCTTAACTAC
TTTGACCCTC ACAGGTTTAC TCACTAAGAA
CTTGAGTTTC AAGAGAAAAG ATGACATGTT
TGCTGCTTAA ACAAGCAATA TCTAAAAGCA
TATTTAGTTA TAAACGTCTT ACCAAGAATT
GATATAATTT TCATTTAAAC ATTTTTATAA
ATAGTAGTTT ACAAGATATA GTAAGTACAT
CTCTAAAAAT ACAGTGTATT CATGTACCTT
GACATAAACT TGTAGTAGTA CCTTAGTTTT
ATTCATGTTG TTATATTAAC TACCATCACT
TTGAATACAT ACCTGTTCAC
B
GTACAGTATA GGTCGGTTTA GGTTTATTGC
CTTAATTGCT TGGTTTTGAG TTAGTACTGT
AGCAAATGCT ATCACACTTT GCATTCCCTA
AAAACAGGTA AATTCATTAA GGAAACAGAC
AAAGTATATA ATAATCTCGC TACATAAATA
TTTCAAGATC AGCTATCTGC ATTCTGATAA
AATTGTTTTT AAAATTTAAG CATTCCTTGG
ACTTTGAATT GTAAGTTGAT CAAATTCAAA
AATGAATTGT TACTGTATTC TTCTCTCCTG
GCCCTAAAAT CTATCTAAAA CATGGCATGG
GGAGTTTCTT AATGTTTCAG TGTCCATTTC
CTGGGTGTTT CCCTCTAGGT TTTTTTTCCT
CACCCCTCAA GCTTCTATGT GGATCCCAGC
TAGAGCTCAT ACTACTTATC CAACACACAT
CATTGTGCAA GCACTCTTTT ATATTCATAC
TAGTACTTTT AAGTGTGTGT GCGGTGGGAA
AAGGTTACCA ATCACATTTT

In some embodiments of the methods of the disclosure, the subject having PrePF or at risk of developing IPF has a mutation in a sequence encoding Family with sequence similarity 13 member A (FAM13A). In some embodiments the polymorphism is rs2609255 comprising (SEQ ID NO: 2).

(SEQ ID NO: 2)
GTATTCATCA ACTCCTATTT CATTCCCTCT TCCTGTGCTC
ACTGGAAGAT GACATTTCCC AGACTTCCAA GAATGTTACT
GAGTTCTGGA ATGTAAGTAG AAGGGATAAG TATCACTTCT
GTGCTGTGGC GGTTATGGAC CTGTGAACTT TGCACACGCC
TTCTATCTTC TTTTTCAGTG TCCATTTCAG AGGGCATGTT
TTCAGATGAA ACCAGTAGAA GATGGAAGCA GCCTGTGACT
AGAATCACTG CTTAGGGTCT TGCTGCCTAG GAATCCCACT
CTACCTGCAA CAGACTGTGA AAGAACCGAG AAATACACTG
ATTTTGAACA TAGCCCATAC TATAATGGGG ATGTTTGTTA
CAGCAGTTAG CATTAAAAAC CTTGGCTAGG CATTGGTCAT
AATTGTAGAA CACAGCAAAT GAAGGGAAAC TGGAACATAG
AGGCCAGTGA GAACTTTAGG GTTAATGAAA AATGAGGGCA
ACCAGGATAA TTTGGTTCTT
K
GCCAAATAGG AAGGTGAAAC CAAAGGTAGA CTGGAGGTCA
GAAAATCAGT CCAGCACATG TGATGTTTTC ATTTAGTTGC
CTGTATGTCT GTCTGGTCTC CAGCTCAGCC TGGCTCCTTG
AGGTAAGAGG CAGTGGCTGT TCACCTTTGC ATCCCAGCAC
CTGGCATACA ATAGATGGGA TGAAATGTTC AAACTGAGCC
TAAGCTTCAG GGTGCTTATC AAAGCAGGGA AGATACACAA
GAGGAGATGA TTCAGGTCCA GGGCAGGTCA GGTATCTAAA
CCCAGTCTCT TAGGAAGCTG GATCCTCCGA ACCAGGGAGA
ACAAGCTGGA TATGCACTGG ATTTCCCAGC AGTACTGATC
TAGAGACTCT CATAGAGTCC CTTTTATTCC TTGGCCTAGG
GTTACAACTG CTTATAGCAT CTGGAAAGAC TCAACACCTC
AAAAGAGACT TTCAGTAGAT ACAGCAAATA CACTCATGGA
ATTGATAATT AAGCTTCAAT

In some embodiments of the methods of the disclosure, the subject having PrePF or at risk of developing IPF has a mutation in a sequence encoding Telomerase Reverse Transcriptase (TERT). In some embodiments the polymorphism is rs2736100 comprising (SEQ ID NO: 3).

(SEQ ID NO: 3)
ATTGTCGTTG TTTGCTTTTG TTTATTGAGA CAGTCTCACT
CTGTCACCCA GGCTGGAGTG TAATGGCACA ATCTCGGCTC
ACTGCAACCT CTGCCTCCTC GGTTCAAGCA GTTCTCATTC
CTCAACCTCA TGAGTAGCTG GGATTACAGG CGCCCACCAC
CACGCCTGGC TAATTTTTGT ATTTTTAGTA GAGATAGGCT
TTCACCATGT TGGCCAGGCT GGTCTCAAAC TCCTGACCTC
AAGTGATCTG CCCGCCTTGG CCTCCCACAG TGCTGGGATT
ACAGGTGCAA GCCACCGTGC CCGGCATACC TTGATCTTTT
AAAATGAAGT CTGAAACATT GCTACCCTTG TCCTGAGCAA
TAAGACCCTT AGTGTATTTT AGCTCTGGCC ACCCCCCAGC
CTGTGTGCTG TTTTCCCTGC TGACTTAGTT CTATCTCAGG
CATCTTGACA CCCCCACAAG CTAAGCATTA TTAATATTGT
TTTCCGTGTT GAGTGTTTCT
K
TAGCTTTGCC CCCGCCCTGC TTTTCCTCCT TTGTTCCCCG
TCTGTCTTCT GTCTCAGGCC CGCCGTCTGG GGTCCCCTTC
CTTGTCCTTT GCGTGGTTCT TCTGTCTTGT TATTGCTGGT
AAACCCCAGC TTTACCTGTG CTGGCCTCCA TGGCATCTAG
CGACGTCCGG GGACCTCTGC TTATGATGCA CAGATGAAGA
TGTGGAGACT CACGAGGAGG GCGGTCATCT TGGCCCGTGA
GTGTCTGGAG CACCACGTGG CCAGCGTTCC TTAGCCAGTG
AGTGACAGCA ACGTCCGCTC GGCCTGGGTT CAGCCTGGAA
AACCCCAGGC ATGTCGGGGT CTGGTGGCTC CGCGGTGTCG
AGTTTGAAAT CGCGCAAACC TGCGGTGTGG CGCCAGCTCT
GACGGTGCTG CCTGGCGGGG GAGTGTCTGC TTCCTCCCTT
CTGCTTGGGA ACCAGGACAA AGGATGAGGC TCCGAGCCGT
TGTCGCCCAA CAGGAGCATG

In some embodiments of the methods of the disclosure, the subject having PrePF or at risk of developing IPF has a mutation in a sequence encoding Desmoplakin (DSP). In some embodiments the polymorphism is rs2076295 comprising (SEQ ID NO: 4).

(SEQ ID NO: 4)
ATTTGGGAAC CTTTAAAAAA TATTCTGGCT TCAAAAATAC
TCCATATTTA CATCTTTGGT TCTATCTGAA GTAAAGCCGT
GATGGTGTGC GTAAGTGAAA CAGGTGCAAA GGGGCAACAA
CAAAGGGCGC CTCTCTTTGT CTTTGTGTCG CAGGCGGAGA
TGGACATGGT GGCCTGGGGT GTGGACCTGG CCTCAGTGGA
GCAGCACATT AACAGCCACC GGGGCATCCA CAACTCCATC
GGCGACTATC GCTGGCAGCT GGACAAAATC AAAGCCGACC
TGGTACTTGT CTGTGTTTCA TTTTAGAGTC TTCAAAATAT
CTACCGAAGG ATCGTGTAAT TACTCAATCC CAGGGAGTTT
CTTCTGAAAC ATTGCTATTA TTTCTTTCCC AGAAGACTGG
AAATGTTTAG AAATCCCACT TCTTAAATGG GGAAGTGGAA
TCAGTAGCCC TATTAGAGAT TATGTTAACA CTTGAAGAGG
AGTTAAACCA GAGGCTGAGG
K
TGTGCAAACA CTCATTTGCA GTTTGTGAAT AAGTCTCTTT
AGGGGTGGCA GTTTGTTTCT GCGGTAAGCA GAACATCTTT
TTGAATAGGG GAAATGCAAC AGTCTTATAC AGTAGTTTGT
GTCATTGGTG AATCCTTTCC TAGGTGGTAA TTAAAACATT
ATTTCTACTG AGCAAAGCCA TATGTCATCC CGACACCCGC
TCCCATGCTG AAAAAAGTCA GACTTGAAAC TGGGTTGAGA
ATTACAGCAT AAAATCATAA CTGATCTTAA GTGCTTAGTT
TCCCGCAGGT CTCTACACTT GTAAATCACT AAACTTTTTT
TTTTTTTTTT TACCTGAGAC CATAGCTTCT CATCCTCATT
TCTTCTTCTG GCTTTTTGGG GCTTACTTTT GTCCACCTGA
GCCCCTGACC AACTTTCTCC TTCATTTCTC TAAGACCTAG
GGAATCCTAA ATGATGTCTT TAAACTTTAA GACAATTTTC
TAACACGTGA GTCTTTAAGT

In some embodiments of the methods of the disclosure, the subject having PrePF or at risk of developing IPF has a mutation in a sequence encoding Zinc-alpha 2-Glycoprotein 1 (AZGP1). In some embodiments the polymorphism is rs4727443 comprising (SEQ ID NO: 5).

(SEQ ID NO: 5)
CCCAACCCAA ATAAGCACTA TAACCTCTTG TTATTCACTT
CTCATGCAAC CAGTCTTCTG TTCTCTGTGA GTCTTTAGGA
AATGAGGAGC ATGATCTTCT AGCAGTAAAA CACCTGTAGA
GAATTGCCTT ATGTTTTTTG TTTGTTTATT TGTTTGTGTG
CTTTGGTTTG GTTTGCTTTT TTTTTTTTTT TTTTTTTTTT
TTTGAGATGG AGTCTCGCCC TGTTGCCCAG GCTGGAGTGT
AGTGGCGAAA TCTCGGCTCA CTGCAACCTC CACCTCCCTG
GTTCAAGCAA TTCCCCTGTC TCAGCCTCCC GAGTAGCTGA
GATTACAGGT GCACACCACC ACGCCCGGCT AATTTTTTTG
TATTTTTAGT AGAGATGGGG TTTCACCATG TTGGCCAGAC
TGGTCTCGAA CTTCTGACCT CAGGCAATCC GCCTGCCTCA
GCCTCCCAAA GCGCTGGGAT TACAGGCATG AGCCACTGCG
CCCCGCCTCC ATGTTAATCA
M
TCTTTCTGAT TTCAAATAAC TCATTATCCC CATGACCTTA
TGGATTTGTT TTTCCTCTTC ATCCACAAAA TTCTCCAGAG
AAGTCTCCCT TGTTATCTCT TGGCTGTGCT TTCTATCTCA
CCAGTTATCT TTCTCCAAAG AGCTTCCTCT GCAAAGAAGC
TTTGTATATG AAGACCATGT GGGGGCTGAA TCAAGACCAA
GTTTCACAAC CTAAAAGTAG TTCACAAAGC TTCCTTGCCT
CTATTCTCTG CAAATCTGTA AACTCTTCAG CTGACCCAAT
TTCTCTCTTT AGCCTTCAGA GATTATTTTA TTTTATTTTA
TTTCATTTCA TTTCATTTCA TTTTGACAGA ATCTAGCTCT
GTCGCCCAGG CTGGAGTGCA GTGGCACCAT CTTTGCTCAC
TGCAACCTCC CCCTCACAGG TTCAAGCAAC TGTCCTGCCT
CAGCCTCCCG AGTAGCTGGG ATTACAGGCG TGAGCCACCA
CGCCCAGCTG ATTTTTTTTT

In some embodiments of the methods of the disclosure, the subject having PrePF or at risk of developing IPF has a mutation in a sequence encoding Oligonucleotide/oligosaccharide-binding Fold Containing 1 (OBFC1). In some embodiments the polymorphism is rs11191865 comprising (SEQ ID NO: 6).

(SEQ ID NO: 6)
CCTCTACTGC CGTACACCCC ACCACTCAGC CTTGGAGTGC
CTGTGTGCAG AGCAGGGCTG AGGCATGGTG CTGCTTTGGT
GGTCTAGGTT TGCTGCAGGG CCAGGTGGCC TGAGCTCCAG
GCAGGATCTC TGGCTGCACT CAGCCCTTTC TGCCTCCCCA
AATGCTCTAT ATCACTATTT GTACACTGAG CAGAGTAAAG
TTAGAGAGAA CTGTTTTATA GAATAGGGCT GGCCCCCGCT
CCCCTGGCCT ACGTGATGGT CCTTCCTGGC TGCCAGGTAC
TTGTTTGTAT TAGAGACAGA CACTCCACAG GGTCTGTTGT
GGCCCACAGC ACATAGGCAA TCAGAGGCAG AAAGCAGAGC
TGTTTGGACC CACAGAGGGC CGGCTGTCTG CCACTGAAAT
GTCTTTCCAG TTGGTTGAGA AGCAGCAGGA TGCTCTGCTG
GTGATGTCTG AAAGTCCCAG GATTCTTTGG GTCTCCAAGG
AGATCCTAGC ATATACCACT
R
TCGTGGTTTT AATAAAGAGC AAAAACACTT TCAGATGGGG
AGAAGAGTGG AACAAAAGGT ATTCTTCCTG GGTTGAAGTC
TGGGGGAAAG GCATTGAGAA GACTGGGCTA ATGGCACAAA
CCAATGAAGT ACTCAAGTCA CCTGTGATGG AGGCCAGTCA
TCCAATGGTA TCAACTTTGT ATGTGGCAAC ACTTAATAAA
AATCTGAACA GGTCTTCACT TGTGGACACA GTAGACTTTC
TTGAAAAAGG ACAGAAAAGT GAGCCCTGTG AATTTTCATC
TCACGGACTG ACAACAATGA CTTGCCTTTA AGGACAGTCA
CTCAAGATGA AGATGCAACA AAACCCTTCC AGTTCCAAGT
GGCTGATGAA AAAAAAAAAA TCTTAAAAGC ATCACAGAAC
AACGGAGAAA GAGATCAGAA GACTATAACA GATAGTTTGA
ATTTTAAAAC TCAGAGAAAA GCAACTGAGG AGGAAATACA
CTGCTTAGAA AGAAGAAACT

In some embodiments of the methods of the disclosure, the subject having PrePF or at risk of developing IPF has a mutation in a sequence encoding Mucin 5B (MUC5B). In some embodiments the polymorphism is rs35705950 comprising (SEQ ID NO: 7).

SEQ ID NO: 7)
TGGACGGCCT CTGAAGGGGT CTGTGGGGTC CTGGACGGGT
CCCCATTCAT GGCAGGATTA ACCCCCCTCG GGTTCTGTGT
GGTCCAGGCC GCCCCTTTGT CTCCACTGCC CCCTGGCCAG
AATGAGGGAC AGTGACCCAC CCAGGGCTGG GCCTGGCTCA
GACTCCGTCA GAGCCGCAGG GCAAGTTCCT GGCACGTCCG
AGGTGGGAGG CTCCTCTGCG CTCCAGGAGG CTGTGCCTGG
CCCCCCTTCC CGGCAGGAAC CGGCTGTGTC CCTTTCCTTC
CTTTATCTTC TGTTTTCAGC
D
CCTTCAACTG TGAAGAGGTG AACTCTTCAA ACACGCTGAG
CAAACAGGCC CGACTCCCAG GGCCGCATCC GGGATGTCTC
AATAGCTGTG GCCTTGACGT CCACCTCGGA CCCCTGCCCC
GGACCCAGCC CAGTTCCCAA TGGGCCCTCT GCCCGGGGAG
GTGCCTAGTG GGAGGGACGA GGGCAAAGTC GGGGCCCCCA
CTTGTTTGGT GTCACTGTGT GCCAGCGGCC ACTGGCGGGC
GAGGCTGTTC CAGGGTGGAG GCGGGGAGGG TTGGACCACA
GGCACTGAGC GGGGACAGAG

In some embodiments of the methods of the disclosure, the subject having PrePF or at risk of developing IPF has a mutation in a sequence encoding ATPase Phospholipid Transporting 11A (ATP11A). In some embodiments the polymorphism is rs12787690 comprising (SEQ ID NO: 8).

(SEQ ID NO: 8)
GTCATTGGTC AAATGTGGCC TGTATCTAAA TTCCAACTGT
TAGAATCATA GACATCTAGA GCTTACGTCA GTTTTAGATA
TTTCTTATGA ATTCTCAGAA TTCATAGATT CTCATTTTTA
TTCTTAGACT TCTCAGATAT TCCGTTTTTG ATAGTATACC
CTTCTGAGTC TAATATGTCC TAAAGTGCGA ACTTGTACAA
TTTttttttt tttttttttt tttttttttt t
K
tgataaggag ttttactctg tcacccaggc tggagtgcag
tgacccgatc tcggctcact gcaacctctg cctcccgggt
tcaagtgatt gtgatgtctc agtctcccaa gtagctggga
ttacaggctc ctgccaccac atgcctagct aattgttata
ctttagtaga aatggggctt cgccgtgtta gtcaggctgg
tcttgtactc ctgacctcag ttgatctgcc taccttggcc
cccaaggtgc tgggattaca ggcatgagcc accgcgcctg
accCAGCTTC TTAAATTATT CTGGGCCACC AGTAATGTGA
ATCATGtaaa ttaaaatata taattaaaCA AAATCATATA
GCGATTAGAG ATAATAGTTG TGAAATGCTT GAAAAATCAT
AGGCATTTAA TAAATAGAAG CCATTCCAAT TAGGATTCTT
CTTGATTTTT TTTCAAGACC AAAAAAATAC TCttttaaat
atttattata ataCTCCATG

In some embodiments of the methods of the disclosure, the subject having PrePF or at risk of developing IPF has a mutation in a sequence encoding Isovaleryl-CoA dehydrogenase (IVD)/Dispatched RND Transporter Family Member 2 (DISP2). In some embodiments the polymorphism is rs2034650 comprising (SEQ ID NO: 9).

(SEQ ID NO: 9)
aggctgcagt tagtcatgac tgcgcgctgc actccagcct
gggtgacaaa gtgaggccct gtctcaaaaa caataaaaaa
TTTAAAAGAG CTGAGCATGG AGGCcacttt gggaggctga
ggcaggcaga tctcttaagc ccaggagtct gagaccagcc
tgggcgacat gatgaagccc catctctaca aaaaatacaa
aaaaattagc tgagctttat ggcaaatccc tgtaatccca
gttacctagg aggcccaggc aggaagatgg cttgagccca
aaaggttgag gctgtagtga gctgtgatca tgaacagagt
gagaccctgt ttcaaaacaa aatgaaaaac aaacaaacaa
aaaaaCCAAG AAAACAAGAA AACAAAAACT ATACAATGAT
GAGCCAAAAA GCAAGATATG GAAGAatata tatatatata
tatatatata tataGTATGA GTCCAGCTAT AGAAAGTTTG
AAATCAGGCA ACCTAAACAA TATTGTTCAG GGATCTATAC
AGAGGCAGGA AGCCATTGAG AAAGGTAAGG GGAGGATTAT
CACCAAATTC AGGATGGTGG CTCCCCTGGG GAGAATATGT
CAAGGAGGGG CACATGGGCT TGGAATACTG TCTTCATTGA
CCTGCGTGTT GGGTACACAG GAGTTTGTTA TTTTTCACAC
TGCATATGTG CATGTATATA CTCTCCCATA TATACCATGC
ATTTCACACA AGAACACAAA GGCTGTGTGG CTCTGCTCTG
CCCCTTTCCC CTTCCAGCTC CCATTCTCGT C
Y
TCAGCTAGCA GAGGAGGGTC AGGGTCTTTT AGCACAGCTT
CCTTCTGTCT CTGAGTGGGT CAGAGGAGTA CGGGGATGAG
GGCCTCCCTT CTGCGGCTGG GCTCTGGCCA CTCCAGGGTG
GGAAGGCCTG GAGAAAACAG GGCCAGGCAA AGCCGGCTGG
CCCTGCTGTT TCTGCCAATG CTGGGATTAG GCCAGGGCTC
TGGCCCACCT GTCATTTCAC TCATTCAGCA TGAACATAGC
CACTGAGCAC TTACTGTGAG CCCCGGGTGC TATTGGGAGA
GTTCAGATAA GTGAGAGAGG GTCTTTGACC TCAAAGATCT
TACAGAGAGG ACCGTATACA CAAATAACAG TATACCAGCA
AAATGTGAGC TAAGTGTCAT GTGACTACTC atctactctt
tcaataaata tttgttgtgc acctattaca tgccaggaac
tgtgctggat ggtgatcatg taaagacagt caaatcacag
tcctagctct cagattcaca gcctgcctaa tgctggggaa
acTGGAAT

In some embodiments of the methods of the disclosure, the subject having PrePF or at risk of developing IPF has a mutation in a sequence encoding Dipeptidyl Peptidase 9 (DPP9). In some embodiments the polymorphism is rs12610495 comprising (SEQ ID NO: 10).

(SEQ ID NO: 10)
CCAGCCAGAA GGGGCGCAGT TTGTTAGTTC AGCTCCTCCT
GAGACAGAAA TAAAGACACG AACCAAAGGA CATCAGCACT
TACAGGGCTC TCAGGTCACA CACAGGATGT CCGCGCCCAC
TGCAGAGCTG CAGGTCCCCT CCAGGGCAGT GGGGAGCCAC
AAGCAGCGTT AGGCAGCGGC TGGGACCAGG ACCGCCTGAG
CACTCAAGAA CCCCCACTGC CCCAAGCACT GCTGGCAGCA
AGCCCAGAAA ACTGAGCCCG GGGAGCTCCT CTGAGCGGCC
TAAGCACCCC TCTAAGCTGT GCTGCCCCAA TTCAAGCCTG
GCTCACGGCA GCAAAGAAAA AATGTGACCT TCGGAGCTCC
CAAAGGGGCC ACCCATAAGC TGAGAGCCTG CCCGGAAGCA
CTTATAGACC CGCGTGGCTT GTTTTCATTG CAAAGAACAA
TAAAAATTAT CTTGCCTCTG ATCACCACTG ATAGCCCAAG
AAGCAAAAAT TCGATCCCGG
D
GATGAGAAAT GAAATGAAAC ATCGCGAGAA ACTTCCAGGA
ATCTTCTGGA TGTGGCTAGA CTCTTTAGCT TGAGCTTCCA
GACAGGCCGA GGCTTGGTGC TGGAGCCTGG CCCTCCGCTG
ACCTCTCTTC TACCCGGGGG CACAGCCCGG ATTGCAGAGA
GGCTGGCGCA AGAGTGAGGG AGCGAGGGCT AGCCTGTGAT
GGGCTTTCTC CACCTAGCAC CACCCTATGC TGTGGCTCAG
GGGAGTCAAG AGTTTACACA GCTGCAGAGA TGGATTCCAG
GCCACTTACT CAAGTCTACC TACTCCTTCC TTCGGCCAAT
CAGCTGGGTG CCTCTGCGGC CTGTGACACC ACCAGCAAAC
AGCTCCAGAC CTCCTAGCAT GGTCTCTGTC AAGGCTGGGT
GGCAGATCTG TGATCTCCTT TTTAAATTTT TCATTTTTTT
TAAGAGATGG GGTCTTGCTA TATTGCCCAG GCTGGTCTCA
AACTCCTGGG CTCCAGCGAT

In some embodiments of the methods of the disclosure, the wild type human MUC5B gene of the disclosure consists of or comprises the nucleic acid sequence (Genbank Accession number: NM_002458.2):

```
(SEQ ID NO: 11)
        1 cacccggccc ggctccctcc ctgcccgtcc ccgtcccccc acccgtgcca gcccccagga
      61 tgggtgcccc gagcgcgtgc cggacgctgg tgttggctct ggcggccatg ctcgtggtgc
    121 cgcaggcaga gacccagggc cctgtggagc cgagctggga gaatgcaggg cacaccatgg
    181 atggcggtgc cccgacgtcc tcgcccaccc ggcgcgtgag ctttgttcca cccgtcactg
    241 tcttccccag cctgagcccc ctgaacccgg cgcacaatgg gcgggtgtgc agcacctggg
    301 gtgacttcca ctacaagacc ttcgacggcg acgtcttccg cttccctggc ctttgcaact
    361 acgtgttctc tgagcactgc cgcgccgcct acgaggactt caacgtccag ctacgccgag
    421 gcctagtggg ctccaggcct gtggtcaccc gtgttgtcat caaggcccag gggctggtgc
    481 tggaggcgtc caacggctcc gtcctcatca atgggcagcg ggaggagctg ccttacagcc
    541 gcactggcct cctggtggag cagagcgggg actacatcaa ggtcagcatc cggctggtgc
    601 tgacattcct gtggaacgga gaggacagtg ccctgctgga gctggatccc aaatacgcca
    661 accagacctg tggcctgtgt ggggacttca acggcctccc ggccttcaac gagttctatg
    721 cccacaacgc caggctgacc ccgctccagt ttgggaacct gcagaagttg gatgggccca
    781 cggagcagtg cccggacccg ctgcccttgc cggccggcaa ctgcacggac gaggagggca
    841 tctgccaccg caccctgctg gggccggcct ttgcggagtg ccacgcactg gtggacagca
    901 ctgcgtacct ggccgcctgc gcccaggacc tgtgccgctg ccccacctgc ccgtgtgcca
    961 cctttgtgga atactcacgc cagtgcgccc acgcgggggg ccagccgcgg aactggaggt
  1021 gccctgagct ctgcccccgg acctgccccc tcaacatgca gcaccaggag tgtggctcac
  1081 cctgcacgga cacctgctcc aacccccagc gcgcgcagct ctgcgaggac cactgtgtgg
  1141 acggctgctt ctgcccccca ggcacggtgc tggatgacat cacgcactct ggctgcctgc
  1201 ccctcgggca gtgcccctgc acccacggcg gccgcaccta cagcccgggc acctccttca
  1261 acaccacctg cagctcctgc acctgctccg gggggctatg gcagtgccag gacctgccgt
  1321 gccctggcac ctgctctgtg cagggcgggg cccacatctc cacctatgat gagaaactct
  1381 acgacctgca tggtgactgc agctacgttc tgtccaagaa atgtgccgac agcagcttca
  1441 ccgtgctggc tgagctgcgg aagtgcggcc tgacggacaa cgagaactgc ctgaaagcgg
  1501 tgacgctcag cctggacggc ggggacacgg ccatccgggt ccaagcggac ggcggcgtgt
  1561 tcctcaactc catctacacg cagctgcccc tgtcggcagc caacatcacc ctgttcacac
  1621 cctcgagctt cttcatcgtg gtgcagacag gcctggggct gcagctgctg gtgcagctgg
  1681 tgccactcat gcaggtgttt gtcaggctgg accccgccca ccagggccag atgtgcggcc
  1741 tgtgtgggaa cttcaaccag aaccaggctg acgacttcac ggccctcagc ggggtggtgg
  1801 aggccacggg cgcagccttc gccaacacct ggaaggccca ggctgcctgt gccaatgcca
  1861 ggaacagctt tgaggacccc tgctccctca gtgtggagaa tgagaactac gcccggcact
  1921 ggtgctcgcg cctgaccgat cccaacagtg ccttctcgcg ctgccactcc atcatcaacc
  1981 ccaagccctt ccactcgaac tgcatgtttg acacctgcaa ctgtgagcgg agcgaggact
  2041 gcctgtgcgc cgcgctgtcc tcctatgtgc acgcctgtgc cgccaagggc gtacagctca
  2101 gcgactggag ggacggcgtc tgcaccaagt acatgcagaa ctgccccaag tcccagcgct
  2161 acgcctacgt ggtggatgcc tgccagccca cttgccgcgg cctgagtgag gccgacgtca
  2221 cctgcagcgt ttccttcgtg cctgtggacg gctgcacctg ccccgcgggc accttcctca
  2281 atgacgcggg cgcctgtgtg cccgcccagg agtgcccctg ctacgctcac ggcaccgtgc
  2341 tggctcctgg agaggtggtg cacgacgagg gcgccgtgtg ttcatgtacg ggtgggaagc
  2401 taagctgcct gggagcctct ctgcagaaaa gcacagggtg tgcagccccc atggtgtacc
  2461 tggactgcag caacagctcg gcgggcaccc ctggggccga gtgcctccgg agctgccaca
  2521 cgctggacgt gggctgtttc agcacacact gcgtgtccgg ctgtgtctgt cccccggggc
  2581 tggtgtcgga tgggagtggg ggctgcattg ccgaggagga ctgcccctgt gtgcacaacg
  2641 aggccaccta caagcctgga gagaccatca gggtcgactg caacacctgc acctgcagga
  2701 accggaggtg ggagtgcagc caccggctct gcctgggcac ctgcgtggcc tacggggatg
  2761 gccacttcat cacctttgat ggcgatcgct acagctttga aggcagctgc gagtacatct
  2821 tggcccagga ctactgtggg gacaacacca cccacgggac cttccgcatc gtcaccgaga
  2881 acatcccctg tgggaccacc ggcaccacct gctccaaggc catcaagctc ttcgtggaga
  2941 gctacgagct gatcctccaa gaggggacct ttaaggcggt ggcgagaggg ccgggtgggg
  3001 acccacccta caagatacgc tacatgggga tcttcctggt catcgagacc cacgggatgg
  3061 ccgtgtcctg ggaccggaag accagcgtgt tcatccgact gcaccaggac tacaagggca
  3121 gggtctgcgg cctgtgcggg aacttcgacg acaatgccat caatgacttt gccacgcgta
  3181 gccggtccgt ggtgggggac gcactggagt ttgggaacag ctggaagctc tccccctcct
  3241 gcccggacgc cctggcaccc aaggacccct gcacggccaa ccccttccgc aagtcctggg
  3301 cccagaagca gtgcagcatc ctccacggcc ccaccttcgc cgcctgccgc tcccaggttg
  3361 actccaccaa gtactacgag gcctgcgtga acgacgcgtg tgcctgcgac tcgggtggcg
  3421 actgcgagtg tttctgcacg gctgtggctg cctacgccca ggcctgccac gacgcgggcc
  3481 tgtgtgtgtc ctggcggact ccggacacct gccccttgtt ctgtgacttc tacaacccac
  3541 atgggggctg tgagtggcac taccagccct gcggggcacc ctgcctaaaa acctgccgga
  3601 accccagtgg gcactgcctg gtggacctgc ctggcctgga aggctgctac ccgaagtgcc
  3661 cacccagcca gcccttcttc aatgaggacc agatgaagtg cgtggcccag tgtggctgct
  3721 acgacaagga cggaaactac tatgacgtcg gtgcaagggt ccccacagcg gagaactgcc
  3781 agagctgtaa ctgcacaccc agtggcatcc agtgcgctca cagccttgag gcctgcacct
  3841 gcacctatga ggacaggacc tacagctacc aggacgtcat ctacaacacc accgatgggc
  3901 ttggcgcctg cttgatcgcc atctgcggaa gcaacggcac catcatcagg aaggctgtgg
  3961 catgtcctgg aactccagcc acaacgccat tcaccttcac caccgcctgg gtcccccact
  4021 ccacgacaag cccggccctc ccggtctcca ccgtgtgtgt ccgcgaggtc tgccgctggt
  4081 ccagctggta caatgggcac cgcccagagc ccggcctggg aggcggagac tttgagacgt
  4141 ttgaaaacct gaggcagaga gggtaccagg tatgccctgt gctggctgac atcgagtgcc
  4201 gggcggcgca gcttcccgac atgccgctgg aggagctggg ccagcaggtg gactgtgacc
  4261 gcatgcgggg gctgatgtgc gccaacagcc aacagagtcc cccgctctgt cacgactacg
  4321 agctgcgggt tctctgctgc gaatacgtgc cctgtggccc ctccccggcc ccaggcacca
  4381 gccctcagcc ctccctcagt gccagcacgg agcctgctgt gcctacccca acccagacca
  4441 cagcaaccga aaagaccacc ctatgggtga ccccgagcat ccggtcgacg gcggccctca
  4501 cctcgcagac tgggtccagc tcaggccccg tgacggtcac cccctcggcc ccaggtacca
  4561 ccacctgcca gccccggtgt cagtggacag agtggtttga tgaggactac cccaagtctg
  4621 aacaacttgg aggggacgtt gagtcctacg ataagatcag ggccgctgga gggcacttat
  4681 gccagcagcc taaggacata gagtgccagg ccgagagctt ccccaactgg accctggcac
  4741 aggtggggca gaaggtgcac tgtgacgtcc acttcggcct ggtgtgcagg aactgggagc
  4801 aggagggcgt cttcaagatg tgctacaact acaggatccg ggtcctctgc tgcagtgacg
  4861 accactgcag gggacgtgcc acaaccccgc caccgaccac agagctggag acggccacca
  4921 ccaccaccac ccaggccctg ttctcaacgc cgcagcctac gagtagcccg gggctgacca
  4981 gggctccccc ggccagcacc acagcagtcc ccaccctctc agaaggactg acatccccca
  5041 gatacacaag cacccttggt acagccacca cgggaggccc cacgacgcct gcaggctcca
  5101 cagaacccac tgtcccaggg gtggccacat ccacccttcc aacacgctca gcccttccag
  5161 ggacgacggg gagcttgggc acatggcgcc cctcacagcc acccacgctg gccccaacaa
  5221 caatggcaac ctccagagct cgcccgacag gcacagccag caccgcttcc aaagagccgc
  5281 tgaccacgag cctggcgcca acactcacga gcgagctgtc cacctctcag gccgagacca
  5341 gcacgcccag gacagagacg acaatgagcc ccttgactaa caccaccacc agccagggca
  5401 cgacccgctg tcaaccgaag tgtgagtgga cagagtggtt tgacgtggac ttcccaacct
  5461 caggggttgc aggcggggac atggaaactt ttgaaaacat cagggctgct gggggcaaga
  5521 tgtgctgggc accaaagagc atagagtgcc gggcggagaa ctaccccgag gtaagcatcg
  5581 accaggtcgg gcaggtgctg acctgcagcc tggagacggg gctgacctgc aagaacgaag
  5641 accagacagg caggttcaac atgtgcttca actacaacgt gcgtgtgctt tgctgtgacg
  5701 actacagcca ctgccccagt accccagcca ccagctccac ggccacgccc tcctcaactc
  5761 cggggacgac ctggatcctc acaaagccga ccacaacagc cactacgact gcgtccactg
  5821 gatccacggc caccccgacc tccaccctga gaacagctcc ccctcccaaa gtgctgacca
  5881 ccacggccac cacacccaca gtcaccagct ccaaagccac tccctcctcc agtccaggga
  5941 ctgcaaccgc ccttccagca ctgagaagca cagccaccac acccacagct accagcgtta
  6001 cacccatccc ctcttcctcc ctgggcacca cctggacccg cctatcacag accaccacac
  6061 ccacggccac catgtccaca gccacaccct cctccactcc agagactgcc cacacctcca
  6121 cagtgcttac cgccacggcc accacaactg gggccaccgg ctctgtggcc accccctcct
  6181 ccaccccagg aacagctcac actaccaaag tgccaactac cacaaccacg ggcttcacag
  6241 ccaccccctc ctccagccca gggacggcac tcacgcctcc agtgtggatc agcacaacca
  6301 ccacacccac aaccagaggc tccacggtga ccccctcctc catcccgggg accacccaca
  6361 ccgccacagt gctgaccacc accaccacaa ctgtggccac tggttctatg gcaacaccct
  6421 cctctagcac acagaccagt ggtactcccc catcactgac caccacggcc actacgatca
  6481 cggccaccgg ctccaccacc aacccctcct caactcctgg gacaactccc atccccccag
  6541 tgctgaccac caccgccacc acacctgcag ccaccagcaa cacagtgact ccctcctctg
  6601 ccctagggac cacccacaca cccccagtgc cgaacaccat ggccaccaca cacgggcgat
  6661 ccctgccccc cagcagtccc cacacggtgc gcacagcctg gacttcggcc acctcgggca
  6721 tcttgggcac cacccacatc acagagcctt ccacggtgac ttcccacacc ctagcagcaa
  6781 ccaccggtac cacccagcac tcgactccag ccctttccag ccctcaccct agcagcagaa
  6841 ccaccgagtc acccccttct ccagggacga ccaccccggg ccacaccacg gccacctcca
  6901 ggaccacagc cacggccaca cccagcaaga cccgcacctc gaccctgctg cccagcagcc
  6961 ccacatcggc ccccataacc acggtggtga ccatgggctg tgagccccag tgtgcctggt
  7021 cagagtggct ggactacagc taccccatgc cggggccctc tggcggggac tttgacacct
  7081 actccaacat ccgtgcggcc ggaggggccg tctgtgagca gcccctgggc ctcgagtgcc
  7141 gtgcccaggc ccagcctggt gtccccctgc gggagttggg ccaggtcgtg gaatgcagcc
  7201 tggactttgg cctggtctgc aggaaccgtg agcaggtggg gaagttcaag atgtgcttca
  7261 actatgaaat ccgtgtgttc tgctgcaact acggccactg ccccagcacc ccggccacca
  7321 gctctacggc catgccctcc tccactccgg ggacgacctg gatcctcaca gagctgacca
  7381 caacagccac tacgactgag tccactggat ccacggccac cccgtcctcc accccaggga
  7441 ccacctggat cctcacagag ccgagcacta cagccaccgt gacggtgccc accggatcca
  7501 cggccaccgc ctcctccacc caggcaactg ctggcacccc acatgtgagc accacggcca
  7561 cgacacccac agtcaccagc tccaaagcca ctcccttctc cagtccaggg actgcaaccg
  7621 cccttccagc actgagaagc acagccacca cacccacagc taccagcttt acagccatcc
  7681 cctcctcctc cctgggcacc acctggaccc gcctatcaca gaccaccaca cccacggcca
  7741 ccatgtccac agccacaccc tcctccactc cagagactgt ccacacctcc acagtgctta
  7801 ccaccacggc caccacaacc ggggccaccg gctctgtggc caccccctcc tccaccccag
  7861 gaacagctca cactaccaaa gtgctgacta ccacaaccac gggcttcaca gccaccccct
  7921 cctccagccc agggacggca cgcacgcttc cagtgtggat cagcacaacc accacaccca
  7981 caaccagagg ttccacggtg accccctcct ccatcccggg gaccacccac acccccacag
  8041 tgctgaccac caccaccaca actgtggcca ctggttctat ggcaacaccc tcctctagca
  8101 cacagaccag tggtactccc ccatcactga ccaccacggc cactacgatc acggccaccg
  8161 gctccaccac caacccctcc tcaactccag ggacaacacc tatcccccca gtgctgacca
  8221 ccaccgccac cacacctgca gccaccagca gcacagtgac tccctcctct gccctaggga
  8281 ccacccacac acccccagtg ccgaacacca cggccaccac acacgggcga tccctgtccc
  8341 ccagcagtcc ccacacggtg cgcacagcct ggacttcggc cacctcaggc accttgggca
  8401 ccacccacat cacagagcct tccacgggga cttcccacac cccagcagca accaccggta
  8461 ccacccagca ctcgactcca gccctgtcca gccctcaccc tagcagcagg accaccgagt
  8521 cacccccttc tccagggacg accaccccgg gccacaccag ggccacctcc aggaccacgg
  8581 ccacggccac acccagcaag acccgcacct cgaccctgct gcccagcagc cccacatcgg
  8641 ccccaataac cacggtggtg accatgggct gtgagcccca gtgtgcctgg tcagagtggc
  8701 tggactacag ctaccccatg ccggggccct ctggcgggga ctttgacacc tactccaaca
  8761 tccgtgcggc cggaggggcc gtctgtgagc agcccctggg cctcgagtgc cgtgcccagg
  8821 cccagcctgg tgtccccctg cgggagttgg gccaggtcgt ggaatgcagc ctggactttg
  8881 gcctggtctg caggaaccgt gagcaggtgg ggaagttcaa gatgtgcttc aactatgaaa
  8941 tccgtgtgtt ctgctgcaac tacggccact gccccagcac cccggccacc agctctacgg
  9001 ccacgccctc ctccactcca gggacgacct ggatcctcac agagcagacc acagcagcca
  9061 ctacgaccgc aaccactgga tccacggcca tcccgtcctc caccccggga acagctcccc
  9121 ctcccaaagt gctgaccagc acggccacca cacccacagc caccagttcc aaagccactt
  9181 cctcctccag tccaaggact gcaaccaccc ttccagtgct gacaagcaca gccaccaaat
  9241 ccacagctac cagctttaca cccatcccct ccttcaccct tgggaccacc gggaccctcc
  9301 cagaacagac caccacaccc atggccacca tgtccacaat ccacccctcc tccactccgg
  9361 agaccaccca cacctccaca gtgctgacca cgaaggccac cacgacaagg gccaccagtt
  9421 ccatgtccac cccctcctcc actccgggga cgacctggat cctcacagag ctgaccacag
  9481 cagccactac aactgcagcc actggcccca cggccacccc gtcctccacc ccagggacca
  9541 cctggatcct cacagagccc agcactacag ccaccgtgac ggtgcccacc ggatccacgg
  9601 ccaccgcctc ctccacccgg gcaactgctg gcaccctcaa agtgctgacc agcacggcca
  9661 ccacacccac agtcatcagc tccagagcca ctccctcctc cagtccaggg actgcaaccg
  9721 cccttccagc actgagaagc acagccacca cacccacagc taccagcgtt acagccatcc
  9781 cctcttcctc cctgggcacc gcctggaccc gcctatcaca gaccaccaca cccacggcca
  9841 ccatgtccac agccacaccc tcctctactc cagagactgt ccacacctcc acagtgctta
  9901 ccaccacgac caccacaacc agggccaccg gctctgtggc caccccctcc tccaccccag
  9961 gaacagctca cactaccaaa gtgccgacta ccacaaccac gggcttcaca gccaccccct
10021 cctccagccc agggacggca ctcacgcctc cagtgtggat cagcacaacc accacaccca
10081 caaccagagg ctccacggtg accccctcct ccatcccggg gaccacccac accgccacag
10141 tgctgaccac caccaccaca actgtggcca ctggttctat ggcaacaccc tcctctagca
10201 cacagaccag tggtactccc ccatcactga ccaccacggc cactacgatc acagccaccg
10261 gctccaccac caacccctcc tcaactccag ggacaactcc catcccccca gtgctgacca
10321 ccaccgccac cacacctgca gccaccagca gcacagtgac tccctcctct gccctaggga
10381 ccacccacac acccccagtg ccgaacacca cggccaccac acacgggcgg tccctgcccc
10441 ccagcagtcc ccacacggtg cgcacagcct ggacttcggc cacctcgggc atcttgggca
10501 ccacccacat cacagagcct tccacggtga cttcccacac cccagcagca accaccagta
10561 ccacccagca ctcgactcca gccctgtcca gccctcaccc tagcagcagg accaccgagt
10621 cacccccttc tccagggacg accaccccgg gccacaccag gggcacctcc aggaccacag
10681 ccacagccac acccagcaag acccgcacct cgaccctgct gcccagcagc cccacatcgg
10741 cccccataac cacggtggtg accacgggct gtgagcccca gtgtgcctgg tcagagtggc
10801 tggactacag ctaccccatg ccggggccct ctggcgggga ctttgacacc tactccaaca
10861 tccgtgcggc cggaggggca gtctgtgagc agcccctggg cctcgagtgc cgtgcccagg
10921 cccagcctgg tgtccccctg cgggagttgg gccaggtcgt ggaatgcagc ctggactttg
10981 gcctggtctg caggaaccgt gagcaggtgg ggaagttcaa gatgtgcttc aactatgaaa
11041 tccgtgtgtt ctgctgcaac tacggccact gccccagcac cccggccacc agctctacgg
11101 ccacgccctc ctcaactccg gggacgacct ggatcctcac aaagctgacc acaacagcca
11161 ctacgactga gtccactgga tccacggcca ccccgtcctc caccccaggg accacctgga
11221 tcctcacaga gccgagcact acagccaccg tgacggtgcc caccggatcc acggccaccg
11281 cctcctccac ccaggcaact gctggcaccc cacatgtgag caccacggcc acgacaccca
11341 cagtcaccag ctccaaagcc actcccttct ccagtccagg gactgcaacc gcccttccag
11401 cactgagaag cacagccacc acacccacag ctaccagctt tacagccatc ccctcctcct
11461 ccctgggcac cacctggacc cgcctatcac agaccaccac acccacggcc accatgtcca
11521 cagccacacc ctcctccact ccagagactg cccacacctc cacagtgctt accaccacgg
11581 ccaccacaac cagggccacc ggctctgtgg ccaccccctc ttccacccca ggaacagctc
11641 acactaccaa agtgccgact accacaacca cgggcttcac agtcaccccc tcctccagcc
11701 cagggacggc acgcacgcct ccagtgtgga tcagcacaac caccacaccc acaaccagtg
11761 gctccacggt gaccccctcc tccgtcccgg ggaccaccca cacccccaca gtgctgacca
11821 ccaccaccac aactgtggcc actggttcta tggcaacacc ctcctctagc acacagacca
11881 gtggtactcc cccatcactg atcaccacgg ccactacgat cacggccacc ggctccacca
11941 ccaacccctc ctcaactcca gggacaacac ctatcccccc agtgctgacc accaccgcca
12001 ccacacctgc agccaccagc agcacagtga ctccctcctc tgccctaggg accacccaca
12061 cacccccagt gccgaacacc acggccacca cacacgggcg atccctgtcc cccagcagtc
12121 cccacacggt gcgcacagcc tggacttcgg ccacctcagg caccttgggc accacccaca
12181 tcacagagcc ttccacgggg acttcccaca ccccagcagc aaccaccggt accacccagc
12241 actcgactcc agccctgtcc agccctcacc ctagcagcag gaccaccgag tcaccccctt
12301 ccccagggac gaccaccccg ggccacacca cggccacctc caggaccacg gccacggcca
12361 cacccagcaa gacccgcacc tcgaccctgc tgcccagcag ccccacatcg gcccccataa
12421 ccacggtggt gaccacgggc tgtgagcccc agtgtgcctg gtcagagtgg ctggactaca
12481 gctaccccat gccggggccc tctggcgggg actttgacac ctactccaac atccgtgcgg
12541 ccggaggggc cgtctgtgag cagcccctgg gcctcgagtg ccgtgcccag gcccagcctg
12601 gtgtccccct gggggagttg ggccaggtcg tggaatgcag cctggacttt ggcctggtct
12661 gcaggaaccg tgagcaggtg gggaagttca agatgtgctt caactatgaa atccgtgtgt
12721 tctgctgcaa ctacggccac tgccccagca ccccggccac cagctctacg gccatgccct
12781 cctccactcc ggggacgacc tggatcctca cagagctgac cacaacagcc actacgactg
12841 catccactgg atccacggcc accccgtcct ccaccccggg aacagctccc cctcccaaag
12901 tgctgaccag cccggccacc acacccacag ccaccagttc caaagccact tcctcctcca
12961 gtccaaggac tgcaaccacc cttccagtgc tgacaagcac agccaccaaa tccacagcta
13021 ccagcgttac acccatcccc tcctccaccc ttgggaccac cgggaccctc ccagaacaga
13081 ccaccacacc cgtggccacc atgtccacaa tccacccctc ctccactccg gagaccaccc
13141 acacctccac agtgctgacc acgaaggcca ccacgacaag ggccaccagt tccacgtcca
13201 ccccctcctc cactccgggg acgacctgga tcctcacaga gctgaccaca gcagccacta
13261 caactgcagc cactggcccc acggccaccc cgtcctccac cccagggacc acctggatcc
13321 tcacagagct gaccacaaca gccactacga ctgcgtccac tggatccacg gccaccccgt
13381 cctccacccc agggaccacc tggatcctca cagagccgag cactacagcc accgtgacgg
13441 tgcccaccgg atccacggcc accgcctcct ccacccaggc aactgctggc accccacatg
13501 tgagcaccac ggccacgaca cccacagtca ccagctccaa agccactccc tcctccagtc
13561 cagggactgc aactgccctt ccagcactga gaagcacagc caccacaccc acagctacca
13621 gctttacagc catcccctcc tcctccctgg gcaccacctg gacccgccta tcacagacca
13681 ccacacccac ggccaccatg tccacagcca caccctcctc cactccagag actgtccaca
13741 cctccacagt gcttaccgcc acggccacca caaccggggc caccggctct gtggccaccc
13801 cctcctccac cccaggaaca gctcacacta ccaaagtgcc gactaccaca accacgggct
13861 tcacagccac cccctcctcc agcccaggga cggcactcac gcctccagtg tggatcagca
13921 caaccaccac acccacaacc accacaccca caaccagtgg ctccacggtg accccctcct
13981 ccatcccggg gaccacccac accgccagag tgctgaccac caccaccaca actgtggcca
14041 ctggttctat ggcaacaccc tcctctagca cacagaccag tggtactccc ccatcactga
14101 ccaccacggc cactacgatc acggccaccg gctccaccac caacccctcc tcaactccag
14161 ggacaacacc catcacccca gtgctgacca gcacggccac cacacccgca gccaccagct
14221 ccaaagccac ttcctcctcc agtccaagga ctgcaaccac ccttccagtg ctgacaagca
14281 cagccacaaa atccacagct accagcttta cacccatccc ctcctccacc ctgtggacca
14341 cgtggaccgt cccagcacag accaccacac ccatgtccac catgtccaca atccacacct
14401 cctctactcc agagaccacc cacacctcca cagtgctgac caccacagcc accatgacaa
14461 gggccaccaa ttccacggcc acaccctcct ccactctggg gacgacccgg atcctcactg
14521 agctgaccac aacagccact acaactgcag ccactggatc cacggccacc ctgtcctcca
14581 ccccagggac cacctggatc ctcacagagc cgagcactat agccaccgtg atggtgccca
14641 ccggttccac ggccaccgcc tcctccactc tgggaacagc tcacaccccc aaagtggtga
14701 ccaccatggc cactatgccc acagccactg cctccacggt tcccagctcg tccaccgtgg
14761 ggaccacccg cacccctgca gtgctcccca gcagcctgcc aaccttcagc gtgtccactg
14821 tgtcctcctc agtcctcacc accctgagac ccactggctt ccccagctcc cacttctcta
14881 ctccctgctt ctgcagggca tttggacagt ttttctcgcc cggggaagtc atctacaata
14941 agaccgaccg agccggctgc catttctacg cagtgtgcaa tcagcactgt gacattgacc
15001 gcttccaggg cgcctgtccc acctccccac cgccagtgtc ctccgccccg ctgtcctcgc
15061 cctcccctgc ccctggctgt gacaatgcca tccctctccg gcaggtgaat gagacctgga
15121 ccctggagaa ctgcacggtg gccaggtgcg tgggtgacaa ccgtgtcgtc ctgctggacc
15181 caaagcctgt ggccaacgtc acctgcgtga acaagcacct gcccatcaaa gtgtcggacc
15241 cgagccagcc ctgtgacttc cactatgagt gcgagtgcat ctgcagcatg tggggcggct
15301 cccactattc cacctttgac ggcacctctt acaccttccg gggcaactgc acctatgtcc
15361 tcatgagaga gatccatgca cgctttggga atctcagcct ctacctggac aaccactact
15421 gcacggcctc tgccactgcc gctgccgccc gctgcccccg cgccctcagc atccactaca
15481 agtccatgga tatcgtcctc actgtcacca tggtgcatgg gaaggaggag ggcctgatcc
15541 tgtttgacca aattccggtg agcagcggtt tcagcaagaa cggcgtgctt gtgtctgtgc
15601 tggggaccac caccatgcgt gtggacattc ctgccctggg cgtgagcgtc accttcaatg
15661 gccaagtctt ccaggcccgg ctgccctaca gcctcttcca caacaacacc gagggccagt
15721 gcggcacctg caccaacaac cagagggacg actgtctcca gcgggacgga accactgccg
15781 ccagttgcaa ggacatggcc aagacgtggc tggtccccga cagcagaaag gatggctgct
15841 gggccccgac tggcacaccc cccactgcca gccccgcagc cccggtgtct agcacaccca
15901 cccccacccc atgcccacca cagccgctct gtgatctgat gctgagccag gtctttgctg
15961 agtgccacaa ccttgtgccc ccgggcccat tcttcaacgc ctgcatcagc gaccactgca
16021 ggggccgcct tgaggtgccc tgccagagcc tggaggctta cgcagagctc tgccgcgccc
16081 ggggagtgtg cagtgactgg cgaggtgcaa ccggtggcct gtgcgacctc acctgcccac
16141 ccaccaaagt gtacaagcca tgcggcccca tacagcctgc cacctgcaac tctaggaacc
16201 agagcccaca gctggagggg atggcggagg gctgcttctg ccctgaggac cagatcctct
16261 tcaacgcaca catgggcatc tgcgtgcagg cctgcccctg cgtgggaccc gatgggtttc
16321 ctaaatttcc cggggagcgg tgggtcagca actgccagtc ctgcgtgtgt gacgagggtt
16381 cagtgtcggt gcagtgcaag cccctgccct gtgacgccca gggtcagccc ccgccgtgca
16441 accgtcccgg cttcgtaacc gtgaccaggc cccgggccga gaacccctgc tgccccgaga
16501 cggtgtgcgt gtgcaacaca accacctgcc cccagagcct gcctgtgtgc ccgccagggc
16561 aggagtccat ctgcacccag gaggagggcg actgctgtcc caccttccgc tgcagacctc
16621 agctgtgttc gtacaatggc accttctacg gggttggtgc aaccttccca ggcgcccttc
16681 cctgccacat gtgtacctgc ctctctgggg acacccagga cccaacggtg caatgtcagg
16741 aggatgcctg caacaatact acctgtcccc agggctttga gtacaagaga gtggccgggc
16801 agtgctgtgg ggagtgcgtc cagaccgcct gcctcacgcc cgatggccag ccagtccagc
16861 tgaatgaaac ctgggtcaac agccatgtgg acaactgcac cgtgtacctc tgtgaggctg
16921 agggtggagt ccatttgctg accccacagc ctgcatcctg cccagatgtg tccagctgca
16981 gggggagcct caggaaaacc ggctgctgct actcctgtga ggaggactcc tgtcaagtcc
17041 gcatcaacac gaccatcctg tggcaccagg gctgcgagac cgaggtcaac atcaccttct
17101 gcgagggctc ctgccccgga gcgtccaagt actcagcaga ggcccaggcc atgcagcacc
17161 agtgcacctg ctgccaggag aggcgggtcc acgaggagac ggtgcccttg cactgtccta
17221 acggctcagc catcctgcac acctacaccc acgtggatga gtgtggctgc acgcccttct
17281 gtgtccctgc gcccatggct cccccacaca cccgtggctt cccggcccag gaggccactg
17341 ctgtctgaga acgttctgcc tccatcccca tgctctgtcc acctggagcc aggatgtgca
17401 ttgtctgatc atgaaaacct tgggcctcct ctgcggagcc ccccggcctg tgtgtggcac
17461 cccgcgctcc gtgctcctgc tgcccacccc gtgggtgaaa ccggccccag aagggtgagg
17521 ggccagcagg acccctttcg ggagggcgcc actcaggagt cctaccctgg gagagcctgt
17581 ggcccacctt ggccttgccc ctccctgatg tcactgggac gccctggaac aaactaagca
17641 tgtgcgggcc tatgtgtccc tgccacggcc ggagcgcccg cgcagcacgg attccagctg
17701 gccacgtccg gccgctgggg cagacaggct ggtccaggca aggccagctg ctgccaggaa
17761 gctgcgacag gcaaggcggc cgcctgtcca tgcctgctgc agggtaactc agggctgagg
17821 tcgcaacggc caggtcagag aggggtcagc atcccaaagc cccctctgct caacccagcc
17881 cagttttgca aataaaccct gagcattgag tacgtt
```

In some embodiments of the methods of the disclosure, the wild type human MUC5B gene of the disclosure consists of or comprises the amino acid sequence (Genbank Accession number: NP_002449.2):

```
(SEQ ID NO: 12)
      1 mgapsacrtl vlalaamlvv pqaetqgpve pswenaghtm dggaptsspt rrvsfvppvt
    61 vfpslsplnp ahngrvcstw gdfhyktfdg dvfrfpglcn yvfsehcraa yedfnvqlrr
  121 glvgsrpvvt rvvikaqglv leasngsvli ngqreelpys rtgllveqsg dyikvsirlv
  181 ltflwngeds alleldpkya nqtcglcgdf nglpafnefy ahnarltplq fgnlqkldgp
  241 teqcpdplpl pagnctdeeg ichrtllgpa faechalvds taylaacaqd lcrcptcpca
  301 tfveysrqca haggqprnwr cpelcprtcp lnmqhqecgs pctdtcsnpq raqlcedhcv
  361 dgcfcppgtv lddithsgcl plgqcpcthg grtyspgtsf nttcssctcs gglwqcqdlp
  421 cpgtcsvqgg ahistydekl ydlhgdcsyv lskkcadssf tvlaelrkcg ltdnenclka
  481 vtlsldggdt airvqadggv flnsiytqlp lsaanitlft pssffivvqt glglqllvql
  541 vplmqvfvrl dpahqgqmcg lcgnfnqnqa ddftalsgvv eatgaafant wkaqaacana
  601 rnsfedpcsl svenenyarh wcsrltdpns afsrchsiin pkpfhsncmf dtcncersed
  661 clcaalssyv hacaakgvql sdwrdgvctk ymqncpksqr yayvvdacqp tcrglseadv
  721 tcsysfvpvd gctcpagtfl ndagacvpaq ecpcyahgtv lapgevvhde gavcsctggk
  781 lsclgaslqk stgcaapmvy ldcsnssagt pgaeclrsch tldvgcfsth cvsgcvcppg
  841 lvsdgsggci aeedcpcvhn eatykpgeti rvdcntctcr nrrwecshrl clgtcvaygd
  901 ghfitfdgdr ysfegsceyi laqdycgdnt thgtfrivte nipcgttgtt cskaiklfve
  961 syelilqegt fkavargpgg dppykirymg iflviethgm ayswdrktsv firlhqdykg
1021 rvcglcgnfd dnaindfatr srsvvgdale fgnswklsps cpdalapkdp ctanpfrksw
1081 aqkqcsilhg ptfaacrsqv dstkyyeacv ndacacdsgg dcecfctava ayaqachdag
1141 lcvswrtpdt cplfcdfynp hggcewhyqp cgapclktcr npsghclvdl pglegcypkc
1201 ppsqpffned qmkcvaqcgc ydkdgnyydv garvptaenc qscnctpsgi qcahsleact
1261 ctyedrtysy qdviynttdg lgacliaicg sngtiirkav acpgtpattp ftfttawvph
1321 sttspalpvs tvcvrevcrw sswynghrpe pglgggdfet fenlrqrgyq vcpvladiec
1381 raaqlpdmpl eelgqqvdcd rmrglmcans qqspplchdy elrvlcceyv pcgpspapgt
1441 spqpslsast epavptptqt tatekttlwv tpsirstaal tsqtgsssgp vtvtpsapgt
1501 ttcqprcqwt ewfdedypks eqlggdvesy dkiraagghl cqqpkdiecq aesfpnwtla
1561 qvgqkvhcdv hfglvcrnwe qegvfkmcyn yrirvlccsd dhcrgrattp pptteletat
1621 ttttqalfst pqptsspglt rappasttav ptlsegltsp rytstlgtat tggpttpags
1681 teptvpgvat stlptrsalp gttgslgtwr psqpptlapt tmatsrarpt gtastaskep
1741 lttslaptlt selstsqaet stprtettms pltntttsqg ttrcqpkcew tewfdvdfpt
1801 sgvaggdmet feniraaggk mcwapksiec raenypevsi dqvgqvltcs letgltckne
1861 dqtgrfnmcf nynvrvlccd dyshcpstpa tsstatpsst pgttwiltkp tttatttast
1921 gstatptstl rtapppkvlt ttattptvts skatpssspg tatalpalrs tattptatsv
1981 tpipssslgt twtrlsqttt ptatmstatp sstpetahts tvltatattt gatgsvatps
2041 stpgtahttk vptttttgft atpssspgta ltppvwistt ttpttrgstv tpssipgtth
2101 tatvlttttt tvatgsmatp ssstqtsgtp psltttatti tatgsttnps stpgttpipp
2161 vltttattpa atsntvtpss algtthtppv pntmatthgr slppssphtv rtawtsatsg
2221 ilgtthitep stvtshtlaa ttgttqhstp alssphpssr ttesppspgt ttpghttats
2281 rttatatpsk trtstllpss ptsapittvv tmgcepqcaw sewldysypm pgpsggdfdt
2341 ysniraagga vceqplglec raqaqpgvpl relgqvvecs ldfglvcrnr eqvgkfkmcf
2401 nyeirvfccn yghcpstpat sstampsstp gttwiltelt ttatttestg statpsstpg
2461 ttwiltepst tatvtvptgs tatasstqat agtphvstta ttptvtsska tpfsspgtat
2521 alpalrstat tptatsftai pssslgttwt rlsqtttpta tmstatpsst petvhtstvl
2581 tttatttgat gsvatpsstp gtahttkvlt ttttgftatp ssspgtartl pvwisttttp
2641 ttrgstvtps sipgtthtpt vlttttttva tgsmatpsss tqtsgtppsl tttattitat
2701 gsttnpsstp gttpippvlt ttattpaats stvtpssalg tthtppvpnt tatthgrsls
2761 pssphtvrta wtsatsgtlg tthitepstg tshtpaattg ttqhstpals sphpssrtte
2821 sppspgtttp ghtratsrtt atatpsktrt stllpsspts apittvvtmg cepqcawsew
2881 ldysypmpgp sggdfdtysn iraaggavce qplglecraq aqpgvplrel gqvvecsldf
2941 glvcrnreqv gkfkmcfnye irvfccnygh cpstpatsst atpsstpgtt wilteqttaa
3001 tttattgsta ipsstpgtap ppkvltstat tptatsskat ssssprtatt 1pvltstatk
3061 statsftpip sftlgttgtl peqtttpmat mstihpsstp etthtstvlt tkatttrats
3121 smstpsstpg ttwilteltt aatttaatgp tatpsstpgt twiltepstt atvtvptgst
3181 atasstrata gtlkvltsta ttptvissra tpssspgtat alpalrstat tptatsvtai
3241 pssslgtawt rlsqtttpta tmstatpsst petvhtstvl tttttttrat gsvatpsstp
3301 gtahttkvpt ttttgftatp ssspgtaltp pvwisttttp ttrgstvtps sipgtthtat
3361 vlttttttva tgsmatpsss tqtsgtppsl tttattitat gsttnpsstp gttpippvlt
3421 ttattpaats stvtpssalg tthtppvpnt tatthgrslp pssphtvrta wtsatsgilg
3481 tthitepstv tshtpaatts ttqhstpals sphpssrtte sppspgtttp ghtrgtsrtt
3541 atatpsktrt stllpsspts apittvvttg cepqcawsew ldysypmpgp sggdfdtysn
3601 iraaggavce qplglecraq aqpgvplrel gqvvecsldf glvcrnreqv gkfkmcfnye
3661 irvfccnygh cpstpatsst atpsstpgtt wiltklttta tttestgsta tpsstpgttw
3721 iltepsttat vtvptgstat asstqatagt phvsttattp tvtsskatpf sspgtatalp
3781 alrstattpt atsftaipss slgttwtrls qtttptatms tatpsstpet ahtstvlttt
3841 atttratgsv atpsstpgta httkvptttt tgftvtpsss pgtartppvw isttttptts
3901 gstvtpssvp gtthtptvlt tttttvatgs matpssstqt sgtppslitt attitatgst
3961 tnpsstpgtt pippvlttta ttpaatsstv tpssalgtth tppvpnttat thgrslspss
4021 phtvrtawts atsgtlgtth itepstgtsh tpaattgttq hstpalssph pssrttespp
4081 spgtttpght tatsrttata tpsktrtstl 1pssptsapi ttvvttgcep qcawsewldy
4141 sypmpgpsgg dfdtysnira aggavceqpl glecraqaqp gvplgelgqv vecsldfglv
4201 crnreqvgkf kmcfnyeirv fccnyghcps tpatsstamp sstpgttwil teltttattt
4261 astgstatps stpgtapppk vltspattpt atsskatsss sprtattlpv ltstatksta
4321 tsvtpipsst lgttgtlpeq tttpvatmst ihpsstpett htstvlttka tttratssts
4381 tpsstpgttw iltelttaat ttaatgptat psstpgttwi lteltttatt tastgstatp
4441 sstpgttwil tepsttatvt vptgstatas stqatagtph vsttattptv tsskatpsss
4501 pgtatalpal rstattptat sftaipsssl gttwtrlsqt ttptatmsta tpsstpetvh
4561 tstvltatat ttgatgsvat psstpgtaht tkvptttttg ftatpssspg taltppvwis
4621 ttttpttttp ttsgstvtps sipgtthtar vlttttttva tgsmatpsss tqtsgtppsl
4681 tttattitat gsttnpsstp gttpitpvlt stattpaats skatsssspr tattlpvlts
4741 tatkstatsf tpipsstlwt twtvpaqttt pmstmstiht sstpetthts tvltttatmt
4801 ratnstatps stlgttrilt eltttattta atgstatlss tpgttwilte pstiatvmvp
4861 tgstatasst lgtahtpkvv ttmatmptat astvpssstv gttrtpavlp sslptfsvst
4921 vsssvlttlr ptgfpsshfs tpcfcrafgq ffspgeviyn ktdragchfy avcnqhcdid
4981 rfqgacptsp ppvssaplss pspapgcdna iplrqvnetw tlenctvarc vgdnrvvlld
5041 pkpvanvtcv nkhlpikvsd psqpcdfhye cecicsmwgg shvstfdgts ytfrgnctyv
5101 lmreiharfg nlslyldnhy ctasataaaa rcpralsihy ksmdivltvt mvhgkeegli
5161 lfdqipvssg fskngvlvsv lgtttmrvdi palgvsvtfn gqvfqarlpy slfhnntegq
5221 cgtctnnqrd dclqrdgtta asckdmaktw lvpdsrkdgc waptgtppta spaapvsstp
5281 tptpcppqpl cdlmlsqvfa echnlvppgp ffnacisdhc rgrlevpcqs leayaelcra
5341 rgvcsdwrga tgglcdltcp ptkvykpcgp iqpatcnsrn qspqlegmae gcfcpedqil
5401 fnahmgicvq acpcvgpdgf pkfpgerwvs ncqscvcdeg svsvqckplp cdaqgqpppc
5461 nrpgfvtvtr praenpccpe tvcvcntttc pqslpvcppg qesictqeeg dccptfrcrp
5521 qlcsyngtfy gvgatfpgal pchmctclsg dtqdptvqcq edacnnttcp qgfeykrvag
5581 qccgecvqta cltpdgqpvq lnetwvnshv dnctvylcea eggvhlltpq pascpdvssc
5641 rgslrktgcc ysceedscqv rinttilwhq gcetevnitf cegscpgask ysaeaqamqh
5701 qctccqerry heetvplhcp ngsailhtyt hvdecgctpf cvpapmapph trgfpaqeat
5761 av
```

In some embodiments of the methods of the disclosure, the wild type human TERT gene of the disclosure consists of or comprises the nucleic acid sequence (Genbank Accession number: NM_198253.2, transcript variant 1):

```
(SEQ ID NO: 13)
      1 caggcagcgc tgcgtcctgc tgcgcacgtg ggaagccctg gccccggcca cccccgcgat
    61 gccgcgcgct ccccgctgcc gagccgtgcg ctccctgctg cgcagccact accgcgaggt
  121 gctgccgctg gccacgttcg tgcggcgcct ggggccccag ggctggcggc tggtgcagcg
  181 cggggacccg gcggctttcc gcgcgctggt ggcccagtgc ctggtgtgcg tgccctggga
  241 cgcacggccg ccccccgccg ccccctcctt ccgccaggtg tcctgcctga aggagctggt
  301 ggcccgagtg ctgcagaggc tgtgcgagcg cggcgcgaag aacgtgctgg ccttcggctt
  361 cgcgctgctg gacggggccc gcgggggccc ccccgaggcc ttcaccacca gcgtgcgcag
  421 ctacctgccc aacacggtga ccgacgcact gcgggggagc ggggcgtggg ggctgctgct
  481 gcgccgcgtg ggcgacgacg tgctggttca cctgctggca cgctgcgcgc tctttgtgct
  541 ggtggctccc agctgcgcct accaggtgtg cgggccgccg ctgtaccagc tcggcgctgc
  601 cactcaggcc cggcccccgc cacacgctag tggaccccga aggcgtctgg gatgcgaacg
  661 ggcctggaac catagcgtca gggaggccgg ggtccccctg ggcctgccag ccccgggtgc
  721 gaggaggcgc gggggcagtg ccagccgaag tctgccgttg cccaagaggc ccaggcgtgg
  781 cgctgcccct gagccggagc ggacgcccgt tgggcagggg tcctgggccc acccgggcag
  841 gacgcgtgga ccgagtgacc gtggtttctg tgtggtgtca cctgccagac ccgccgaaga
  901 agccacctct ttggagggtg cgctctctgg cacgcgccac tcccacccat ccgtgggccg
  961 ccagcaccac gcgggccccc catccacatc gcggccacca cgtccctggg acacgccttg
1021 tcccccggtg tacgccgaga ccaagcactt cctctactcc tcaggcgaca aggagcagct
1081 gcggccctcc ttcctactca gctctctgag gcccagcctg actggcgctc ggaggctcgt
1141 ggagaccatc tttctgggtt ccaggccctg gatgccaggg actccccgca ggttgccccg
1201 cctgccccag cgctactggc aaatgcggcc cctgtttctg gagctgcttg ggaaccacgc
1261 gcagtgcccc tacggggtgc tcctcaagac gcactgcccg ctgcgagctg cggtcacccc
1321 agcagccggt gtctgtgccc gggagaagcc ccagggctct gtggcggccc ccgaggagga
1381 ggacacagac ccccgtcgcc tggtgcagct gctccgccag cacagcagcc cctggcaggt
1441 gtacggcttc gtgcgggcct gcctgcgccg gctggtgccc ccaggcctct ggggctccag
1501 gcacaacgaa cgccgcttcc tcaggaacac caagaagttc atctccctgg ggaagcatgc
1561 caagctctcg ctgcaggagc tgacgtggaa gatgagcgtg cgggactgcg cttggctgcg
1621 caggagccca ggggttggct gtgttccggc cgcagagcac cgtctgcgtg aggagatcct
1681 ggccaagttc ctgcactggc tgatgagtgt gtacgtcgtc gagctgctca ggtctttctt
1741 ttatgtcacg gagaccacgt ttcaaaagaa caggctcttt ttctaccgga agagtgtctg
1801 gagcaagttg caaagcattg gaatcagaca gcacttgaag agggtgcagc tgcgggagct
1861 gtcggaagca gaggtcaggc agcatcggga agccaggccc gccctgctga cgtccagact
1921 ccgcttcatc cccaagcctg acgggctgcg gccgattgtg aacatggact acgtcgtggg
1981 agccagaacg ttccgcagag aaaagagggc cgagcgtctc acctcgaggg tgaaggcact
2041 gttcagcgtg ctcaactacg agcgggcgcg gcgccccggc ctcctgggcg cctctgtgct
2101 gggcctggac gatatccaca gggcctggcg caccttcgtg ctgcgtgtgc gggcccagga
2161 cccgccgcct gagctgtact ttgtcaaggt ggatgtgacg ggcgcgtacg acaccatccc
2221 ccaggacagg ctcacggagg tcatcgccag catcatcaaa ccccagaaca cgtactgcgt
2281 gcgtcggtat gccgtggtcc agaaggccgc ccatgggcac gtccgcaagg ccttcaagag
2341 ccacgtctct accttgacag acctccagcc gtacatgcga cagttcgtgg ctcacctgca
2401 ggagaccagc ccgctgaggg atgccgtcgt catcgagcag agctcctccc tgaatgaggc
2461 cagcagtggc ctcttcgacg tcttcctacg cttcatgtgc caccacgccg tgcgcatcag
2521 gggcaagtcc tacgtccagt gccaggggat cccgcagggc tccatcctct ccacgctgct
2581 ctgcagcctg tgctacggcg acatggagaa caagctgttt gcggggattc ggcgggacgg
2641 gctgctcctg cgtttggtgg atgatttctt gttggtgaca cctcacctca cccacgcgaa
2701 aaccttcctc aggaccctgg tccgaggtgt ccctgagtat ggctgcgtgg tgaacttgcg
2761 gaagacagtg gtgaacttcc ctgtagaaga cgaggccctg ggtggcacgg cttttgttca
2821 gatgccggcc cacggcctat tcccctggtg cggcctgctg ctggataccc ggaccctgga
2881 ggtgcagagc gactactcca gctatgcccg gacctccatc agagccagtc tcaccttcaa
2941 ccgcggcttc aaggctggga ggaacatgcg tcgcaaactc tttggggtct tgcggctgaa
3001 gtgtcacagc ctgtttctgg atttgcaggt gaacagcctc cagacggtgt gcaccaacat
3061 ctacaagatc ctcctgctgc aggcgtacag gtttcacgca tgtgtgctgc agctcccatt
3121 tcatcagcaa gtttggaaga accccacatt tttcctgcgc gtcatctctg acacggcctc
3181 cctctgctac tccatcctga aagccaagaa cgcagggatg tcgctggggg ccaagggcgc
3241 cgccggccct ctgccctccg aggccgtgca gtggctgtgc caccaagcat tcctgctcaa
3301 gctgactcga caccgtgtca cctacgtgcc actcctgggg tcactcagga cagcccagac
3361 gcagctgagt cggaagctcc cggggacgac gctgactgcc ctggaggccg cagccaaccc
3421 ggcactgccc tcagacttca agaccatcct ggactgatgg ccacccgccc acagccaggc
3481 cgagagcaga caccagcagc cctgtcacgc cgggctctac gtcccaggga gggaggggcg
3541 gcccacaccc aggcccgcac cgctgggagt ctgaggcctg agtgagtgtt tggccgaggc
3601 ctgcatgtcc ggctgaaggc tgagtgtccg gctgaggcct gagcgagtgt ccagccaagg
3661 gctgagtgtc cagcacacct gccgtcttca cttccccaca ggctggcgct cggctccacc
3721 ccagggccag cttttcctca ccaggagccc ggcttccact ccccacatag gaatagtcca
3781 tccccagatt cgccattgtt cacccctcgc cctgccctcc tttgccttcc acccccacca
3841 tccaggtgga gaccctgaga aggaccctgg gagctctggg aatttggagt gaccaaaggt
3901 gtgccctgta cacaggcgag gaccctgcac ctggatgggg gtccctgtgg gtcaaattgg
3961 ggggaggtgc tgtgggagta aaatactgaa tatatgagtt tttcagtttt gaaaaaaa
```

In some embodiments of the methods of the disclosure, the wild type human TERT gene of the disclosure consists of or comprises the amino acid sequence (Genbank Accession number: NP_937983.2, transcript variant 1):

```
(SEQ ID NO: 14)
      1 mpraprcrav rsllrshyre vlplatfvrr lgpqgwrlvq rgdpaafral vaqclvcvpw
    61 darpppaaps frqvsclkel varvlqrlce rgaknvlafg falldgargg ppeafttsvr
  121 sylpntvtda lrgsgawgll lrrvgddvlv hllarcalfv lvapscayqv cgpplyqlga
  181 atqarpppha sgprrrlgce rawnhsvrea gvplglpapg arrrggsasr slplpkrprr
  241 gaapepertp vgqgswahpg rtrgpsdrgf cvvsparpae eatslegals gtrhshpsvg
  301 rqhhagppst srpprpwdtp cppvyaetkh flyssgdkeq lrpsfllssl rpsltgarrl
  361 vetiflgsrp wmpgtprrlp rlpqrywqmr plflellgnh aqcpygvllk thcplraavt
  421 paagvcarek pqgsvaapee edtdprrlvq llrqhsspwq vygfvraclr rlvppglwgs
  481 rhnerrflrn tkkfislgkh aklslqeltw kmsvrdcawl rrspgvgcvp aaehrlreei
  541 lakflhwlms vyvvellrsf fyvtettfqk nrlffyrksv wsklqsigir qhlkrvqlre
  601 lseaevrqhr earpalltsr lrfipkpdgl rpivnmdyvv gartfrrekr aerltsrvka
  661 lfsvinyera rrpgllgasv lglddihraw rtfvlrvraq dpppelyfvk vdvtgaydti
  721 pqdrltevia siikpqntyc vrryavvqka ahghvrkafk shvstltdlq pymrqfvahl
  781 qetsplrdav vieqssslne assglfdvfl rfmchhavri rgksyvqcqg ipqgsilstl
  841 lcslcygdme nklfagirrd glllrlvddf llvtphltha ktflrtivrg vpeygcvvnl
  901 rktvvnfpve dealggtafv qmpahglfpw cglildtrtl evqsdyssya rtsirasltf
  961 nrgfkagrnm rrklfgvlrl kchslfldlq vnslqtvctn iykilllqay rfhacvlqlp
1021 fhqqvwknpt fflrvisdta slcysilkak nagmslgakg aagplpseav qwlchqafll
1081 kltrhrvtyv pllgslrtaq tqlsrklpgt tltaleaaan palpsdfkti ld
```

In some embodiments of the methods of the disclosure, the wild type human TERT gene of the disclosure consists of or comprises the nucleic acid sequence (Genbank Accession number: NM_001193376.1, transcript variant 2):

```
(SEQ ID NO: 15)
      1 caggcagcgc tgcgtcctgc tgcgcacgtg ggaagccctg gccccggcca cccccgcgat
    61 gccgcgcgct ccccgctgcc gagccgtgcg ctccctgctg cgcagccact accgcgaggt
  121 gctgccgctg gccacgttcg tgcggcgcct ggggccccag ggctggcggc tggtgcagcg
  181 cggggacccg gcggctttcc gcgcgctggt ggcccagtgc ctggtgtgcg tgccctggga
  241 cgcacggccg ccccccgccg ccccctcctt ccgccaggtg tcctgcctga aggagctggt
  301 ggcccgagtg ctgcagaggc tgtgcgagcg cggcgcgaag aacgtgctgg ccttcggctt
  361 cgcgctgctg gacggggccc gcgggggccc ccccgaggcc ttcaccacca gcgtgcgcag
  421 ctacctgccc aacacggtga ccgacgcact gcgggggagc ggggcgtggg ggctgctgct
  481 gcgccgcgtg ggcgacgacg tgctggttca cctgctggca cgctgcgcgc tctttgtgct
  541 ggtggctccc agctgcgcct accaggtgtg cgggccgccg ctgtaccagc tcggcgctgc
  601 cactcaggcc cggcccccgc cacacgctag tggaccccga aggcgtctgg gatgcgaacg
  661 ggcctggaac catagcgtca gggaggccgg ggtccccctg ggcctgccag ccccgggtgc
  721 gaggaggcgc gggggcagtg ccagccgaag tctgccgttg cccaagaggc ccaggcgtgg
  781 cgctgcccct gagccggagc ggacgcccgt tgggcagggg tcctgggccc acccgggcag
  841 gacgcgtgga ccgagtgacc gtggtttctg tgtggtgtca cctgccagac ccgccgaaga
  901 agccacctct ttggagggtg cgctctctgg cacgcgccac tcccacccat ccgtgggccg
  961 ccagcaccac gcgggccccc catccacatc gcggccacca cgtccctggg acacgccttg
1021 tcccccggtg tacgccgaga ccaagcactt cctctactcc tcaggcgaca aggagcagct
1081 gcggccctcc ttcctactca gctctctgag gcccagcctg actggcgctc ggaggctcgt
1141 ggagaccatc tttctgggtt ccaggccctg gatgccaggg actccccgca ggttgccccg
1201 cctgccccag cgctactggc aaatgcggcc cctgtttctg gagctgcttg ggaaccacgc
1261 gcagtgcccc tacggggtgc tcctcaagac gcactgcccg ctgcgagctg cggtcacccc
1321 agcagccggt gtctgtgccc gggagaagcc ccagggctct gtggcggccc ccgaggagga
1381 ggacacagac ccccgtcgcc tggtgcagct gctccgccag cacagcagcc cctggcaggt
1441 gtacggcttc gtgcgggcct gcctgcgccg gctggtgccc ccaggcctct ggggctccag
1501 gcacaacgaa cgccgcttcc tcaggaacac caagaagttc atctccctgg ggaagcatgc
1561 caagctctcg ctgcaggagc tgacgtggaa gatgagcgtg cgggactgcg cttggctgcg
1621 caggagccca ggggttggct gtgttccggc cgcagagcac cgtctgcgtg aggagatcct
1681 ggccaagttc ctgcactggc tgatgagtgt gtacgtcgtc gagctgctca ggtctttctt
1741 ttatgtcacg gagaccacgt ttcaaaagaa caggctcttt ttctaccgga agagtgtctg
1801 gagcaagttg caaagcattg gaatcagaca gcacttgaag agggtgcagc tgcgggagct
1861 gtcggaagca gaggtcaggc agcatcggga agccaggccc gccctgctga cgtccagact
1921 ccgcttcatc cccaagcctg acgggctgcg gccgattgtg aacatggact acgtcgtggg
1981 agccagaacg ttccgcagag aaaagagggc cgagcgtctc acctcgaggg tgaaggcact
2041 gttcagcgtg ctcaactacg agcgggcgcg gcgccccggc ctcctgggcg cctctgtgct
2101 gggcctggac gatatccaca gggcctggcg caccttcgtg ctgcgtgtgc gggcccagga
2161 cccgccgcct gagctgtact ttgtcaaggt ggatgtgacg ggcgcgtacg acaccatccc
2221 ccaggacagg ctcacggagg tcatcgccag catcatcaaa ccccagaaca cgtactgcgt
2281 gcgtcggtat gccgtggtcc agaaggccgc ccatgggcac gtccgcaagg ccttcaagag
2341 ccacgtctct accttgacag acctccagcc gtacatgcga cagttcgtgg ctcacctgca
2401 ggagaccagc ccgctgaggg atgccgtcgt catcgagcag agctcctccc tgaatgaggc
2461 cagcagtggc ctcttcgacg tcttcctacg cttcatgtgc caccacgccg tgcgcatcag
2521 gggcaagtcc tacgtccagt gccaggggat cccgcagggc tccatcctct ccacgctgct
2581 ctgcagcctg tgctacggcg acatggagaa caagctgttt gcggggattc ggcgggacgg
2641 gctgctcctg cgtttggtgg atgatttctt gttggtgaca cctcacctca cccacgcgaa
2701 aaccttcctc agctatgccc ggacctccat cagagccagt ctcaccttca accgcggctt
2761 caaggctggg aggaacatgc gtcgcaaact ctttggggtc ttgcggctga agtgtcacag
2821 cctgtttctg gatttgcagg tgaacagcct ccagacggtg tgcaccaaca tctacaagat
2881 cctcctgctg caggcgtaca ggtttcacgc atgtgtgctg cagctcccat ttcatcagca
2941 agtttggaag aaccccacat ttttcctgcg cgtcatctct gacacggcct ccctctgcta
3001 ctccatcctg aaagccaaga acgcagggat gtcgctgggg gccaagggcg ccgccggccc
3061 tctgccctcc gaggccgtgc agtggctgtg ccaccaagca ttcctgctca agctgactcg
3121 acaccgtgtc acctacgtgc cactcctggg gtcactcagg acagcccaga cgcagctgag
3181 tcggaagctc ccggggacga cgctgactgc cctggaggcc gcagccaacc cggcactgcc
3241 ctcagacttc aagaccatcc tggactgatg gccacccgcc cacagccagg ccgagagcag
3301 acaccagcag ccctgtcacg ccgggctcta cgtcccaggg agggaggggc ggcccacacc
3361 caggcccgca ccgctgggag tctgaggcct gagtgagtgt ttggccgagg cctgcatgtc
3421 cggctgaagg ctgagtgtcc ggctgaggcc tgagcgagtg tccagccaag ggctgagtgt
3481 ccagcacacc tgccgtcttc acttccccac aggctggcgc tcggctccac cccagggcca
3541 gcttttcctc accaggagcc cggcttccac tccccacata ggaatagtcc atccccagat
3601 tcgccattgt tcacccctcg ccctgccctc ctttgccttc cacccccacc atccaggtgg
3661 agaccctgag aaggaccctg ggagctctgg gaatttggag tgaccaaagg tgtgccctgt
3721 acacaggcga ggaccctgca cctggatggg ggtccctgtg ggtcaaattg gggggaggtg
3781 ctgtgggagt aaaatactga atatatgagt ttttcagttt tgaaaaaaa
```

In some embodiments of the methods of the disclosure, the wild type human TERT gene of the disclosure consists of or comprises the amino acid sequence (Genbank Accession number: NP_001180305.1, transcript variant 2):

```
(SEQ ID NO: 16)
      1 mpraprcrav rsllrshyre vlplatfvrr lgpqgwrlvq rgdpaafral vaqclvcvpw
    61 darpppaaps frqvsclkel varvlqrlce rgaknvlafg falldgargg ppeafttsvr
  121 sylpntvtda lrgsgawgll lrrvgddvlv hllarcalfv lvapscayqv cgpplyqlga
  181 atqarpppha sgprrrlgce rawnhsvrea gvplglpapg arrrggsasr slplpkrprr
  241 gaapepertp vgqgswahpg rtrgpsdrgf cvvsparpae eatslegals gtrhshpsvg
  301 rqhhagppst srpprpwdtp cppvyaetkh flyssgdkeq lrpsfllssl rpsltgarrl
  361 vetiflgsrp wmpgtprrlp rlpqrywqmr plflellgnh aqcpygvllk thcplraavt
  421 paagvcarek pqgsvaapee edtdprrlvq llrqhsspwq vygfvraclr rlvppglwgs
  481 rhnerrflrn tkkfislgkh aklslqeltw kmsvrdcawl rrspgvgcvp aaehrlreei
  541 lakflhwlms vyvvellrsf fyvtettfqk nrlffyrksv wsklqsigir qhlkrvqlre
  601 lseaevrqhr earpalltsr lrfipkpdgl rpivnmdyvv gartfrrekr aerltsrvka
  661 lfsvlnyera rrpgllgasv lglddihraw rtfvlrvraq dpppelyfvk vdvtgaydti
  721 pqdrltevia siikpqntyc vrryavvqka ahghvrkafk shvstltdlq pymrqfvahl
  781 qetsplrdav vieqssslne assglfdvfl rfmchhavri rgksyvqcqg ipqgsilstl
  841 lcslcygdme nklfagirrd glllrlvddf llvtphltha ktflsyarts irasltfnrg
  901 fkagrnmrrk lfgvlrlkch slfldlqvns lqtvctniyk illlqayrfh acvlqlpfhq
  961 qvwknptffl rvisdtaslc ysilkaknag mslgakgaag plpseavqwl chqafllklt
1021 rhrvtyvpll gslrtaqtql srklpgttlt aleaaanpal psdfktild
```

In some embodiments of the methods of the disclosure, the wild type human FAM13A gene of the disclosure consists of or comprises the nucleic acid sequence (Genbank Accession number: NM_014883.3, transcript variant 1):

| (SEQ ID NO: 36) | |
|---|---|
| 1 | atcaaatttc aactccaggc agtccttcca gccatgtggg ttcagcggaa agagaagcaa |
| 61 | aaccactctt cctaaaatgt tagaagctgc tcttcgctta ccttggggcc tttgcattgg |
| 121 | gagctgtttt tcacatcaaa gaatatgtgc tgaatggaat tttagtattt tgctgtcgtt |
| 181 | ttaatatttt cgtctggtct tcctcagttc ttccagacgc tttctgagag aatgggggca |
| 241 | ggagctctag ccatctgtca aagtaaagca gcggttcggc tgaaagaaga catgaaaaag |
| 301 | atagtggcag tgccattaaa tgaacagaag gattttacct atcagaagtt atttggagtc |
| 361 | agtctccaag aacttgaacg gcaggggctc accgagaatg gcattccagc agtagtgtgg |
| 421 | aatatagtgg aatatttgac gcagcatgga cttacccaag aaggtctttt tagggtgaat |
| 481 | ggtaacgtga aggtggtgga acaacttcga ctgaagttcg agagtggagt gcccgtggag |
| 541 | ctcgggaagg acggtgatgt ctgctcagca gccagtctgt tgaagctgtt tctgagggag |
| 601 | ctgcctgaca gtctgatcac ctcagcgttg cagcctcgat tcattcaact ctttcaggat |
| 661 | ggcagaaatg atgttcagga gagtagctta agagacttaa taaaagagct gccagacacc |
| 721 | cactactgcc tcctcaagta cctttgccag ttcttgacaa aagtagccaa gcatcatgtg |
| 781 | cagaatcgca tgaatgttca caatctcgcc actgtatttg ggccaaattg ctttcatgtg |
| 841 | ccacctgggc ttgaaggcat gaaggaacag gacctgtgca acaagataat ggctaaaatt |
| 901 | ctagaaaatt acaataccct gtttgaagta gagtatacag aaaatgatca tctgagatgt |
| 961 | gaaaacctgg ctaggcttat catagtaaaa gaggtctatt ataagaactc cctgcccatc |
| 1021 | cttttaacaa gaggcttaga aagagacatg ccaaaaccac ctccaaaaac caagatccca |
| 1081 | aaatccagga gtgagggatc tattcaggcc cacagagtac tgcaaccaga gctatctgat |
| 1141 | ggcattcctc agctcagctt gcggctaagt tatagaaaag cctgcttgga agacatgaat |
| 1201 | tcagcagagg gtgctattag tgccaagttg gtacccagtt cacaggaaga tgaaagacct |
| 1261 | ctgtcacctt tctatttgag tgctcatgta ccccaagtca gcaatgtgtc tgcaaccgga |
| 1321 | gaactcttag aaagaaccat ccgatcagct gtagaacaac atctttttga tgttaataac |
| 1381 | tctggaggtc aaagttcaga ggactcagaa tctggaacac tatcagcatc ttctgccaca |
| 1441 | tctgccagac agcgccgccg ccagtccaag gagcaggatg aagttcgaca tgggagagac |
| 1501 | aagggactta tcaacaaaga aaatactcct tctgggttca accaccttga tgattgtatt |
| 1561 | ttgaatactc aggaagtcga aaaggtacac aaaaatactt ttggttgtgc tggagaaagg |
| 1621 | agcaagccta aacgtcagaa atccagtact aaactttctg agcttcatga caatcaggac |
| 1681 | ggtcttgtga atatggaaag tctcaattcc acacgatctc atgagagaac tggacctgat |
| 1741 | gattttgaat ggatgtctga tgaaaggaaa ggaaatgaaa aagatggtgg acacactcag |
| 1801 | cattttgaga gccccacaat gaagatccag gagcatccca gcctatctga caccaaacag |
| 1861 | cagagaaatc aagatgccgg tgaccaggag gagagctttg tctccgaagt gccccagtcg |
| 1921 | gacctgactg cattgtgtga tgaaaagaac tgggaagagc ctatccctgc tttctcctcc |
| 1981 | tggcagcggg agaacagtga ctctgatgaa gcccacctct cgccgcaggc tgggcgcctg |
| 2041 | atccgtcagc tgctggacga agacagcgac cccatgctct ctcctcggtt ctacgcttat |
| 2101 | gggcagagca ggcaatacct ggatgacaca gaagtgcctc cttccccacc aaactcccat |
| 2161 | tctttcatga ggcggcgaag ctcctctctg gggtcctatg atgatgagca agaggacctg |
| 2221 | acacctgccc agctcacacg aaggattcag agccttaaaa agaagatccg gaagtttgaa |
| 2281 | gatagattcg aagaagagaa gaagtacaga ccttcccaca gtgacaaagc agccaatccg |
| 2341 | gaggttctga aatggacaaa tgaccttgcc aaattccgga gacaacttaa agaatcaaaa |
| 2401 | ctaaagatat ctgaagagga cctaactccc aggatgcggc agcgaagcaa cacactcccc |
| 2461 | aagagttttg gttcccaact tgagaaagaa gatgagaaga agcaagagct ggtggataaa |
| 2521 | gcaataaagc ccagtgttga agccacattg gaatctattc agaggaagct ccaggagaag |
| 2581 | cgagcggaaa gcagccgccc tgaggacatt aaggatatga ccaaagacca gattgctaat |
| 2641 | gagaaagtgg ctctgcagaa agctctgtta tattatgaaa gcattcatgg acggccggta |
| 2701 | acaaagaacg aacggcaggt gatgaagcca ctatacgaca ggtaccggct ggtcaaacag |
| 2761 | atcctctccc gagctaacac catacccatc attggttccc cctccagcaa gcggagaagc |
| 2821 | cctttgctgc agccaattat cgagggcgaa actgcttcct tcttcaagga gataaaggaa |
| 2881 | gaagaggagg ggtcagaaga cgatagcaat gtgaagccag acttcatggt cactctgaaa |
| 2941 | accgatttca gtgcacgatg ctttctggac caattcgaag atgacgctga tggatttatt |
| 3001 | tccccaatgg atgataaaat accatcaaaa tgcagccagg acacagggct ttcaaatctc |
| 3061 | catgctgcct caatacctga actcctggaa cacctccagg aaatgagaga agaaaagaaa |
| 3121 | aggattcgaa agaaacttcg ggattttgaa gacaactttt tcagacagaa tggaagaaat |
| 3181 | gtccagaagg aagaccgcac tcctatggct gaagaataca gtgaatataa gcacataaag |
| 3241 | gcgaaactga ggctcctgga ggtgctcatc agcaagagag acactgattc caagtccatg |
| 3301 | tgaggggcat ggccaagcac agggggctgg cagctgcggt gagagtttac tgtccccaga |
| 3361 | gaaagtgcag ctctggaagg cagccttggg gctggccctg caaagcatgc agcccttctg |
| 3421 | cctctagacc atttggcatc ggctcctgtt tccattgcct gccttagaaa ctggctggaa |
| 3481 | gaagacaatg tgacctgact taggcatttt gtaattggaa agtcaagact gcagtatgtg |
| 3541 | cacatgcgca cgcgcatgca cgcacacaca cacacagtag tggagctttc ctaacactag |
| 3601 | cagagattaa tcactacatt agacaacact catctacaga gaatatacac tgttcttccc |
| 3661 | tggataactg agaaacaaga gaccattctc tgtctaactg tgataaaaac aagctcagga |
| 3721 | ctttattcta tagagcaaac ttgctgtgga gggccatgct ctccttggac ccagttaact |
| 3781 | gcaaacgtgc attggagccc tatttgctgc cgctgccatt ctagtgacct ttccacagag |
| 3841 | ctgcgccttc ctcacgtgtg tgaaaggttt tccccttcag ccctcaggta gatggaagct |
| 3901 | gcatctgccc acgatggcag tgcagtcatc atcttcagga tgtttcttca ggacttcctc |
| 3961 | agctgacaag gaattttggt ccctgcctag gaccgggtca tctgcagagg acagagagat |
| 4021 | ggtaagcagc tgtatgaatg ctgattttaa aaccaggtca tgggagaaga gcctggagat |
| 4081 | tctttcctga acactgactg cacttaccag tctgatttta tcgtcaaaca ccaagccagg |
| 4141 | ctagcatgct catggcaatc tgtttggggc tgttttgttg tggcactagc caaacataaa |
| 4201 | ggggcttaag tcagcctgca tacagaggat cggggagaga aggggcctgt gttctcagcc |
| 4261 | tcctgagtac ttaccagagt ttaatttttt taaaaaaaat ctgcactaaa atccccaaac |
| 4321 | tgacaggtaa atgtagccct cagagctcag cccaaggcag aatctaaatc acactatttt |
| 4381 | cgagatcatg tataaaaaga aaaaaaagaa gtcatgctgt gtggccaatt ataatttttt |
| 4441 | tcaaagactt tgtcacaaaa ctgtctatat tagacatttt ggagggacca ggaaatgtaa |
| 4501 | gacaccaaat cctccatctc ttcagtgtgc ctgatgtcac ctcatgattt gctgttactt |
| 4561 | ttttaactcc tgcgccaagg acagtgggtt ctgtgtccac ctttgtgctt tgcgaggccg |
| 4621 | agcccaggca tctgctcgcc tgccacggct gaccagagaa ggtgcttcag gagctctgcc |
| 4681 | ttagacgacg tgttacagta tgaacacaca gcagaggcac cctcgtatgt tttgaaagtt |
| 4741 | gccttctgaa agggcacagt tttaaggaaa agaaaaagaa tgtaaaacta tactgacccg |
| 4801 | ttttcagttt taaagggtcg tgagaaactg gctggtccaa tgggatttac agcaacattt |
| 4861 | tccattgctg aagtgaggta gcagctctct tctgtcagct gaatgttaag gatggggaaa |
| 4921 | aagaatgcct ttaagtttgc tcttaatcgt atggaagctt gagctatgtg ttggaagtgc |
| 4981 | cctggtttta atccatacac aaagacggta cataatccta caggtttaaa tgtacataaa |
| 5041 | aatatagttt ggaattcttt gctctactgt ttacattgca gattgctata atttcaagga |
| 5101 | gtgagattat aaataaaatg atgcacttta ggatgtttcc tatttttgaa atctgaacat |
| 5161 | gaatcattca catgaccaaa aattgtgttt ttttaaaaat acatgtctag tctgtccttt |
| 5221 | aatagctctc ttaaataagc tatgatatta atcagatcat taccagttag cttttaaagc |
| 5281 | acatttgttt aagactatgt ttttggaaaa atacgctaca gaattttttt ttaagctaca |
| 5341 | aataaatgag atgctactaa ttgttttgga atctgttgtt tctgccaaag gtaaattaac |
| 5401 | taaagattta ttcaggaatc cccatttgaa tttgtatgat tcaataaaag aaaacaccaa |
| 5461 | gtaagttata taaaataaat tgtgtatgag atgttgtgtt ttcctttgta atttccacta |
| 5521 | actaactaac taacttatat tcttcatgga atggagccca gaagaaatga gaggaagccc |
| 5581 | ttttcacact agatcttatt tgaagaaatg tttgttagtc agtcagtcag tggtttctgg |
| 5641 | ctctgccgag ggagatgtgt tccccagcaa ccatttctgc agcccagaat ctcaaggcac |
| 5701 | tagaggcggt gtcttaatta attggcttca caaagacaaa atgctctgga ctgggatttt |
| 5761 | tcctttgctg tgttgggaat atgtgtttat taattagcac atgccaacaa aataaatgtc |
| 5821 | aagagttatt tcataagtgt aagtaaactt aagaattaaa gagtgcagac ttataatttt |
| 5881 | ca |

In some embodiments of the methods of the disclosure, the wild type human FAM13A gene of the disclosure consists of or comprises the amino acid sequence (Genbank Accession number: NP_055698.2, transcript variant 1):

| (SEQ ID NO: 37) | |
|---|---|
| 1 | mgagalaicq skaavrlked mkkivavpln eqkdftyqkl fgvslqeler qgltengipa |
| 61 | vvwniveylt qhgltqeglf rvngnvkvve qlrlkfesgv pvelgkdgdv csaasllklf |
| 121 | lrelpdslit salqprfiql fqdgrndvqe sslrdlikel pdthycllky lcqfltkvak |
| 181 | hhvqnrmnvh nlatvfgpnc fhvppglegm keqdlcnkim akilenyntl feveytendh |
| 241 | lrcenlarli ivkevyykns lpilltrgle rdmpkpppkt kipksrsegs iqahrvlqpe |
| 301 | lsdgipqlsl rlsyrkacle dmnsaegais aklvpssqed erplspfyls ahvpqvsnvs |
| 361 | atgellerti rsaveqhlfd vnnsggqsse dsesgtlsas satsarqrrr qskeqdevrh |
| 421 | grdkglinke ntpsgfnhld dcilntqeve kvhkntfgca gerskpkrqk sstklselhd |
| 481 | nqdglvnmes lnstrshert gpddfewmsd erkgnekdgg htqhfesptm kigehpslsd |
| 541 | tkqqrnqdag dqeesfvsev pqsdltalcd eknweepipa fsswqrensd sdeahlspqa |
| 601 | grlirqllde dsdpmlsprf yaygqsrqyl ddtevppspp nshsfmrrrs sslgsyddeq |
| 661 | edltpaqltr riqslkkkir kfedrfeeek kyrpshsdka anpevlkwtn dlakfrrqlk |
| 721 | esklkiseed ltprmrqrsn tlpksfgsql ekedekkqel vdkaikpsve atlesiqrkl |
| 781 | qekraessrp edikdmtkdq ianekvalqk allyyesihg rpvtknerqv mkplydryrl |
| 841 | vkqilsrant ipiigspssk rrspllqpii egetasffke ikeeeegsed dsnvkpdfmv |
| 901 | tlktdfsarc fldqfeddad gfispmddki pskcsqdtgl snlhaasipe llehlqemre |
| 961 | ekkrirkklr dfednffrqn grnvqkedrt pmaeeyseyk hikaklrlle vliskrdtds |
| 1021 | ksm |

In some embodiments of the methods of the disclosure, the wild type human FAM13A gene of the disclosure consists of or comprises the nucleic acid sequence (Genbank Accession number: NM_001015045.2, transcript variant 2):

| (SEQ ID NO: 17) | |
|---|---|
| 1 | attgaggagc agaaggagta gggtgcgggg gaggaggagg agcgccttta gtgctgcagc |
| 61 | agctgctgct ctgattggcc cggtggttca gctgcttccc tggaacaaaa ggtcaaagtg |
| 121 | gactgcagtg taaatgtaga gaagcagccg ataaaatagc attgcctgaa gaagtttgga |
| 181 | ggctgagagc agcagtagac tggccaactg cagagcaagt tgtttctcca gccgtgcggt |
| 241 | gcagcctcat gcccccaacc cagcttagcc actgtaagaa gacgttcact gtacagacga |
| 301 | ccaaacttgc cgtggaagag acagttgtga gattcccttg caaatttaca tacgagaatg |
| 361 | gcttgtgaaa tcatgcctct gcaaagttca caggaagatg aaagacctct gtcacctttc |
| 421 | tatttgagtg ctcatgtacc ccaagtcagc aatgtgtctg caaccggaga actcttagaa |
| 481 | agaaccatcc gatcagctgt agaacaacat ctttttgatg ttaataactc tggaggtcaa |
| 541 | agttcagagg actcagaatc tggaacacta tcagcatctt ctgccacatc tgccagacag |
| 601 | cgccgccgcc agtccaagga gcaggatgaa gttcgacatg ggagagacaa gggacttatc |
| 661 | aacaaagaaa atactccttc tgggttcaac caccttgatg attgtatttt gaatactcag |
| 721 | gaagtcgaaa aggtacacaa aaatactttt ggttgtgctg gagaaaggag caagcctaaa |
| 781 | cgtcagaaat ccagtactaa actttctgag cttcatgaca atcaggacgg tcttgtgaat |
| 841 | atggaaagtc tcaattccac acgatctcat gagagaactg gacctgatga ttttgaatgg |
| 901 | atgtctgatg aaaggaaagg aaatgaaaaa gatggtggac acactcagca ttttgagagc |
| 961 | cccacaatga agatccagga gcatcccagc ctatctgaca ccaaacagca gagaaatcaa |
| 1021 | gatgccggtg accaggagga gagctttgtc tccgaagtgc cccagtcgga cctgactgca |
| 1081 | ttgtgtgatg aaaagaactg ggaagagcct atccctgctt tctcctcctg gcagcgggag |
| 1141 | aacagtgact ctgatgaagc ccacctctcg ccgcaggctg ggcgcctgat ccgtcagctg |
| 1201 | ctggacgaag acagcgaccc catgctctct cctcggttct acgcttatgg gcagagcagg |
| 1261 | caatacctgg atgacacaga agtgcctcct tccccaccaa actcccattc tttcatgagg |
| 1321 | cggcgaagct cctctctggg gtcctatgat gatgagcaag aggacctgac acctgcccag |
| 1381 | ctcacacgaa ggattcagag ccttaaaaag aagatccgga agtttgaaga tagattcgaa |
| 1441 | gaagagaaga agtacagacc ttcccacagt gacaaagcag ccaatccgga ggttctgaaa |
| 1501 | tggacaaatg accttgccaa attccggaga caacttaaag aatcaaaact aaagatatct |
| 1561 | gaagaggacc taactcccag gatgcggcag cgaagcaaca cactccccaa gagttttggt |
| 1621 | tcccaacttg agaaagaaga tgagaagaag caagagctgg tggataaagc aataaagccc |
| 1681 | agtgttgaag ccacattgga atctattcag aggaagctcc aggagaagcg agcggaaagc |
| 1741 | agccgccctg aggacattaa ggatatgacc aaagaccaga ttgctaatga gaaagtggct |
| 1801 | ctgcagaaag ctctgttata ttatgaaagc attcatggac ggccggtaac aaagaacgaa |
| 1861 | cggcaggtga tgaagccact atacgacagg taccggctgg tcaaacagat cctctcccga |
| 1921 | gctaacacca tacccatcat tggttccccc tccagcaagc ggagaagccc tttgctgcag |
| 1981 | ccaattatcg agggcgaaac tgcttccttc ttcaaggaga taaaggaaga agaggagggg |
| 2041 | tcagaagacg atagcaatgt gaagccagac ttcatggtca ctctgaaaac cgatttcagt |
| 2101 | gcacgatgct ttctggacca attcgaagat gacgctgatg gatttatttc cccaatggat |
| 2161 | gataaaatac catcaaaatg cagccaggac acagggcttt caaatctcca tgctgcctca |
| 2221 | atacctgaac tcctggaaca cctccaggaa atgagagaag aaaagaaaag gattcgaaag |
| 2281 | aaacttcggg attttgaaga caactttttc agacagaatg gaagaaatgt ccagaaggaa |
| 2341 | gaccgcactc ctatggctga agaatacagt gaatataagc acataaaggc gaaactgagg |
| 2401 | ctcctggagg tgctcatcag caagagagac actgattcca agtccatgtg aggggcatgg |
| 2461 | ccaagcacag ggggctggca gctgcggtga gagtttactg tccccagaga aagtgcagct |
| 2521 | ctggaaggca gccttggggc tggccctgca aagcatgcag cccttctgcc tctagaccat |
| 2581 | ttggcatcgg ctcctgtttc cattgcctgc cttagaaact ggctggaaga agacaatgtg |
| 2641 | acctgactta ggcattttgt aattggaaag tcaagactgc agtatgtgca catgcgcacg |
| 2701 | cgcatgcacg cacacacaca cacagtagtg gagctttcct aacactagca gagattaatc |
| 2761 | actacattag acaacactca tctacagaga atatacactg ttcttccctg gataactgag |
| 2821 | aaacaagaga ccattctctg tctaactgtg ataaaaacaa gctcaggact ttattctata |
| 2881 | gagcaaactt gctgtggagg gccatgctct ccttggaccc agttaactgc aaacgtgcat |
| 2941 | tggagcccta tttgctgccg ctgccattct agtgaccttt ccacagagct gcgccttcct |
| 3001 | cacgtgtgtg aaaggttttc cccttcagcc ctcaggtaga tggaagctgc atctgcccac |
| 3061 | gatggcagtg cagtcatcat cttcaggatg tttcttcagg acttcctcag ctgacaagga |
| 3121 | attttggtcc ctgcctagga ccgggtcatc tgcagaggac agagagatgg taagcagctg |
| 3181 | tatgaatgct gattttaaaa ccaggtcatg ggagaagagc ctggagattc tttcctgaac |
| 3241 | actgactgca cttaccagtc tgattttatc gtcaaacacc aagccaggct agcatgctca |
| 3301 | tggcaatctg tttggggctg ttttgttgtg gcactagcca aacataaagg ggcttaagtc |
| 3361 | agcctgcata cagaggatcg gggagagaag gggcctgtgt tctcagcctc ctgagtactt |
| 3421 | accagagttt aattttttta aaaaaaatct gcactaaaat ccccaaactg acaggtaaat |
| 3481 | gtagccctca gagctcagcc caaggcagaa tctaaatcac actattttcg agatcatgta |
| 3541 | taaaaagaaa aaaaagaagt catgctgtgt ggccaattat aatttttttc aaagactttg |
| 3601 | tcacaaaact gtctatatta gacattttgg agggaccagg aaatgtaaga caccaaatcc |
| 3661 | tccatctctt cagtgtgcct gatgtcacct catgatttgc tgttactttt ttaactcctg |
| 3721 | cgccaaggac agtgggttct gtgtccacct ttgtgctttg cgaggccgag cccaggcatc |
| 3781 | tgctcgcctg ccacggctga ccagagaagg tgcttcagga gctctgcctt agacgacgtg |
| 3841 | ttacagtatg aacacacagc agaggcaccc tcgtatgttt tgaaagttgc cttctgaaag |
| 3901 | ggcacagttt taaggaaaag aaaaagaatg taaaactata ctgacccgtt ttcagtttta |
| 3961 | aagggtcgtg agaaactggc tggtccaatg ggatttacag caacattttc cattgctgaa |
| 4021 | gtgaggtagc agctctcttc tgtcagctga atgttaagga tggggaaaaa gaatgccttt |
| 4081 | aagtttgctc ttaatcgtat ggaagcttga gctatgtgtt ggaagtgccc tggttttaat |
| 4141 | ccatacacaa agacggtaca taatcctaca ggtttaaatg tacataaaaa tatagtttgg |
| 4201 | aattctttgc tctactgttt acattgcaga ttgctataat ttcaaggagt gagattataa |
| 4261 | ataaaatgat gcactttagg atgtttccta tttttgaaat ctgaacatga atcattcaca |
| 4321 | tgaccaaaaa ttgtgttttt ttaaaaatac atgtctagtc tgtcctttaa tagctctctt |
| 4381 | aaataagcta tgatattaat cagatcatta ccagttagct tttaaagcac atttgtttaa |
| 4441 | gactatgttt ttggaaaaat acgctacaga attttttttt aagctacaaa taaatgagat |
| 4501 | gctactaatt gttttggaat ctgttgtttc tgccaaaggt aaattaacta aagatttatt |
| 4561 | caggaatccc catttgaatt tgtatgattc aataaaagaa aacaccaagt aagttatata |
| 4621 | aaataaattg tgtatgagat gttgtgtttt cctttgtaat ttccactaac taactaacta |
| 4681 | acttatattc ttcatggaat ggagcccaga agaaatgaga ggaagccctt ttcacactag |
| 4741 | atcttatttg aagaaatgtt tgttagtcag tcagtcagtg gtttctggct ctgccgaggg |
| 4801 | agatgtgttc cccagcaacc atttctgcag cccagaatct caaggcacta gaggcggtgt |
| 4861 | cttaattaat tggcttcaca aagacaaaat gctctggact gggatttttc ctttgctgtg |
| 4921 | ttgggaatat gtgtttatta attagcacat gccaacaaaa taaatgtcaa gagttatttc |
| 4981 | ataagtgtaa gtaaacttaa gaattaaaga gtgcagactt ataattttca |

In some embodiments of the methods of the disclosure, the wild type human FAM13A gene of the disclosure consists of or comprises the amino acid sequence (Genbank Accession number: NP_001015045.1, transcript variant 2):

| (SEQ ID NO: 18) | |
|---|---|
| 1 | maceimplqs sqederplsp fylsahvpqv snvsatgell ertirsaveq hlfdvnnsgg |
| 61 | qssedsesgt lsassatsar qrrrqskeqd evrhgrdkgl inkentpsgf nhlddcilnt |
| 121 | qevekvhknt fgcagerskp krqksstkls elhdnqdglv nmeslnstrs hertgpddfe |
| 181 | wmsderkgne kdgghtqhfe sptmkigehp slsdtkqqrn qdagdqeesf vsevpqsdlt |
| 241 | alcdeknwee pipafsswqr ensdsdeahl spqagrlirq lldedsdpml sprfyaygqs |
| 301 | rqylddtevp psppnshsfm rrrssslgsy ddeqedltpa qltrriqslk kkirkfedrf |
| 361 | eeekkyrpsh sdkaanpevl kwtndlakfr rqlkesklki seedltprmr qrsntlpksf |
| 421 | gsqlekedek kqelvdkaik psveatlesi qrklqekrae ssrpedikdm tkdqianekv |
| 481 | alqkallyye sihgrpvtkn erqvmkplyd ryrlvkqils rantipiigs psskrrspll |
| 541 | qpiiegetas ffkeikeeee gseddsnvkp dfmvtlktdf sarcfldqfe ddadgfispm |
| 601 | ddkipskcsq dtglsnlhaa sipellehlq emreekkrir kklrdfednf frqngrnvqk |
| 661 | edrtpmaeey seykhikakl rllevliskr dtdsksm |

In some embodiments of the methods of the disclosure, the wild type human FAM13A gene of the disclosure consists of or comprises the nucleic acid sequence (Genbank Accession number: NM001265578.1, transcript variant 3):

| (SEQ ID NO: 38) | |
|---|---|
| 1 | attgaggagc agaaggagta gggtgcgggg gaggaggagg agcgccttta gtgctgcagc |
| 61 | agctgctgct ctgattggcc cggtggttca gctgcttccc tggaacaaaa ggtcaaagtg |
| 121 | gactgcagtg taaatgtaga gaagcagccg ataaaatagc attgcctgaa gaagtttgga |
| 181 | ggctgagagc agcagtagac tggccaactg cagagcaagt tgtttctcca gccgtgcggt |
| 241 | gcagcctcat gcccccaacc cagcttagcc actgtaagaa gacgttcact gtacagacga |
| 301 | ccaaacttgc cgtggaagag acagttgtga gattcccttg caaatttaca tacgagaatg |
| 361 | gcttgtgaaa tcatgcctct gcaaagtgct catgtacccc aagtcagcaa tgtgtctgca |
| 421 | accggagaac tcttagaaag aaccatccga tcagctgtag aacaacatct ttttgatgtt |
| 481 | aataactctg gaggtcaaag ttcagaggac tcagaatctg gaacactatc agcatcttct |
| 541 | gccacatctg ccagacagcg ccgccgccag tccaaggagc aggatgaagt tcgacatggg |
| 601 | agagacaagg gacttatcaa caaagaaaat actccttctg ggttcaacca ccttgatgat |
| 661 | tgtattttga atactcagga agtcgaaaag gtacacaaaa atacttttgg ttgtgctgga |
| 721 | gaaaggagca agcctaaacg tcagaaatcc agtactaaac tttctgagct tcatgacaat |
| 781 | caggacggtc ttgtgaatat ggaaagtctc aattccacac gatctcatga gagaactgga |
| 841 | cctgatgatt ttgaatggat gtctgatgaa aggaaaggaa atgaaaaaga tggtggacac |
| 901 | actcagcatt ttgagagccc cacaatgaag atccaggagc atcccagcct atctgacacc |
| 961 | aaacagcaga gaaatcaaga tgccggtgac caggaggaga gctttgtctc cgaagtgccc |
| 1021 | cagtcggacc tgactgcatt gtgtgatgaa aagaactggg aagagcctat ccctgctttc |
| 1081 | tcctcctggc agcgggagaa cagtgactct gatgaagccc acctctcgcc gcaggctggg |
| 1141 | cgcctgatcc gtcagctgct ggacgaagac agcgacccca tgctctctcc tcggttctac |
| 1201 | gcttatgggc agagcaggca atacctggat gacacagaag tgcctccttc cccaccaaac |
| 1261 | tcccattctt tcatgaggcg gcgaagctcc tctctggggt cctatgatga tgagcaagag |
| 1321 | gacctgacac ctgcccagct cacacgaagg attcagagcc ttaaaaagaa gatccggaag |
| 1381 | tttgaagata gattcgaaga agagaagaag tacagacctt cccacagtga caaagcagcc |
| 1441 | aatccggagg ttctgaaatg gacaaatgac cttgccaaat tccggagaca acttaaagaa |
| 1501 | tcaaaactaa agatatctga agaggaccta actcccagga tgcggcagcg aagcaacaca |
| 1561 | ctccccaaga gttttggttc ccaacttgag aaagaagatg agaagaagca agagctggtg |
| 1621 | gataaagcaa taaagcccag tgttgaagcc acattggaat ctattcagag gaagctccag |
| 1681 | gagaagcgag cggaaagcag ccgccctgag gacattaagg atatgaccaa agaccagatt |
| 1741 | gctaatgaga aagtggctct gcagaaagct ctgttatatt atgaaagcat tcatggacgg |
| 1801 | ccggtaacaa agaacgaacg gcaggtgatg aagccactat acgacaggta ccggctggtc |
| 1861 | aaacagatcc tctcccgagc taacaccata cccatcattg gttccccctc cagcaagcgg |
| 1921 | agaagccctt tgctgcagcc aattatcgag ggcgaaactg cttccttctt caaggagata |
| 1981 | aaggaagaag aggaggggtc agaagacgat agcaatgtga agccagactt catggtcact |
| 2041 | ctgaaaaccg atttcagtgc acgatgcttt ctggaccaat tcgaagatga cgctgatgga |
| 2101 | tttatttccc caatggatga taaaatacca tcaaaatgca gccaggacac agggctttca |
| 2161 | aatctccatg ctgcctcaat acctgaactc ctggaacacc tccaggaaat gagagaagaa |
| 2221 | aagaaaagga ttcgaaagaa acttcgggat tttgaagaca actttttcag acagaatgga |
| 2281 | agaaatgtcc agaaggaaga ccgcactcct atggctgaag aatacagtga atataagcac |
| 2341 | ataaaggcga aactgaggct cctggaggtg ctcatcagca agagagacac tgattccaag |
| 2401 | tccatgtgag gggcatggcc aagcacaggg ggctggcagc tgcggtgaga gtttactgtc |
| 2461 | cccagagaaa gtgcagctct ggaaggcagc cttggggctg gccctgcaaa gcatgcagcc |
| 2521 | cttctgcctc tagaccattt ggcatcggct cctgtttcca ttgcctgcct tagaaactgg |
| 2581 | ctggaagaag acaatgtgac ctgacttagg cattttgtaa ttggaaagtc aagactgcag |
| 2641 | tatgtgcaca tgcgcacgcg catgcacgca cacacacaca cagtagtgga gctttcctaa |
| 2701 | cactagcaga gattaatcac tacattagac aacactcatc tacagagaat atacactgtt |
| 2761 | cttccctgga taactgagaa acaagagacc attctctgtc taactgtgat aaaaacaagc |
| 2821 | tcaggacttt attctataga gcaaacttgc tgtggagggc catgctctcc ttggacccag |
| 2881 | ttaactgcaa acgtgcattg gagccctatt tgctgccgct gccattctag tgacctttcc |
| 2941 | acagagctgc gccttcctca cgtgtgtgaa aggttttccc cttcagccct caggtagatg |
| 3001 | gaagctgcat ctgcccacga tggcagtgca gtcatcatct tcaggatgtt tcttcaggac |
| 3061 | ttcctcagct gacaaggaat tttggtccct gcctaggacc gggtcatctg cagaggacag |
| 3121 | agagatggta agcagctgta tgaatgctga ttttaaaacc aggtcatggg agaagagcct |
| 3181 | ggagattctt tcctgaacac tgactgcact taccagtctg attttatcgt caaacaccaa |
| 3241 | gccaggctag catgctcatg gcaatctgtt tggggctgtt ttgttgtggc actagccaaa |
| 3301 | cataaagggg cttaagtcag cctgcataca gaggatcggg gagagaaggg gcctgtgttc |
| 3361 | tcagcctcct gagtacttac cagagtttaa tttttttaaa aaaaatctgc actaaaatcc |
| 3421 | ccaaactgac aggtaaatgt agccctcaga gctcagccca aggcagaatc taaatcacac |
| 3481 | tattttcgag atcatgtata aaaagaaaaa aaagaagtca tgctgtgtgg ccaattataa |
| 3541 | tttttttcaa agactttgtc acaaaactgt ctatattaga cattttggag ggaccaggaa |
| 3601 | atgtaagaca ccaaatcctc catctcttca gtgtgcctga tgtcacctca tgatttgctg |
| 3661 | ttactttttt aactcctgcg ccaaggacag tgggttctgt gtccaccttt gtgctttgcg |
| 3721 | aggccgagcc caggcatctg ctcgcctgcc acggctgacc agagaaggtg cttcaggagc |
| 3781 | tctgccttag acgacgtgtt acagtatgaa cacacagcag aggcaccctc gtatgttttg |
| 3841 | aaagttgcct tctgaaaggg cacagtttta aggaaaagaa aaagaatgta aaactatact |
| 3901 | gacccgtttt cagttttaaa gggtcgtgag aaactggctg gtccaatggg atttacagca |
| 3961 | acattttcca ttgctgaagt gaggtagcag ctctcttctg tcagctgaat gttaaggatg |
| 4021 | gggaaaaaga atgcctttaa gtttgctctt aatcgtatgg aagcttgagc tatgtgttgg |
| 4081 | aagtgccctg gttttaatcc atacacaaag acggtacata atcctacagg tttaaatgta |
| 4141 | cataaaaata tagtttggaa ttctttgctc tactgtttac attgcagatt gctataattt |
| 4201 | caaggagtga gattataaat aaaatgatgc actttaggat gtttcctatt tttgaaatct |
| 4261 | gaacatgaat cattcacatg accaaaaatt gtgttttttt aaaaatacat gtctagtctg |
| 4321 | tcctttaata gctctcttaa ataagctatg atattaatca gatcattacc agttagcttt |
| 4381 | taaagcacat ttgtttaaga ctatgttttt ggaaaaatac gctacagaat ttttttttaa |
| 4441 | gctacaaata aatgagatgc tactaattgt tttggaatct gttgtttctg ccaaaggtaa |
| 4501 | attaactaaa gatttattca ggaatcccca tttgaatttg tatgattcaa taaaagaaaa |
| 4561 | caccaagtaa gttatataaa ataaattgtg tatgagatgt tgtgttttcc tttgtaattt |
| 4621 | ccactaacta actaactaac ttatattctt catggaatgg agcccagaag aaatgagagg |
| 4681 | aagccctttt cacactagat cttatttgaa gaaatgtttg ttagtcagtc agtcagtggt |
| 4741 | ttctggctct gccgagggag atgtgttccc cagcaaccat ttctgcagcc cagaatctca |
| 4801 | aggcactaga ggcggtgtct taattaattg gcttcacaaa gacaaaatgc tctggactgg |
| 4861 | gatttttcct ttgctgtgtt gggaatatgt gtttattaat tagcacatgc caacaaaata |
| 4921 | aatgtcaaga gttatttcat aagtgtaagt aaacttaaga attaaagagt gcagacttat |
| 4981 | aattttca |

In some embodiments of the methods of the disclosure, the wild type human FAM13A gene of the disclosure consists of or comprises the amino acid sequence (Genbank Accession number: NP_001252507.1, transcript variant 3):

| (SEQ ID NO: 39) | |
|---|---|
| 1 | maceimplqs ahvpqvsnvs atgellerti rsaveqhlfd vnnsggqsse dsesgtlsas |
| 61 | satsarqrrr qskeqdevrh grdkglinke ntpsgfnhld dcilntqeve kvhkntfgca |
| 121 | gerskpkrqk sstklselhd nqdglvnmes lnstrshert gpddfewmsd erkgnekdgg |
| 181 | htqhfesptm kigehpslsd tkqqrnqdag dqeesfvsev pqsdltalcd eknweepipa |
| 241 | fsswqrensd sdeahlspqa grlirqllde dsdpmlsprf yayggsrqyl ddtevppspp |
| 301 | nshsfmrrrs sslgsyddeq edltpaqltr riqslkkkir kfedrfeeek kyrpshsdka |
| 361 | anpevlkwtn dlakfrrqlk esklkiseed ltprmrqrsn tlpksfgsql ekedekkqel |
| 421 | vdkaikpsve atlesiqrkl qekraessrp edikdmtkdq ianekvalqk allyyesihg |
| 481 | rpvtknerqv mkplydryrl vkqilsrant ipiigspssk rrspllqpii egetasffke |
| 541 | ikeeeegsed dsnvkpdfmv tlktdfsarc fldqfeddad gfispmddki pskcsqdtgl |
| 601 | snlhaasipe llehlqemre ekkrirkklr dfednffrqn grnvqkedrt pmaeeyseyk |
| 661 | hikaklrlle vliskrdtds ksm |

In some embodiments of the methods of the disclosure, the wild type human FAM13A gene of the disclosure consists of or comprises the nucleic acid sequence (Genbank Accession number: NM_001265579.1, transcript variant 4):

| (SEQ ID NO: 40) | |
|---|---|
| 1 | attgaggagc agaaggagta gggtgcgggg gaggaggagg agcgccttta gtgctgcagc |
| 61 | agctgctgct ctgattggcc cggtggttca gctgcttccc tggaacaaaa ggtcaaagtg |
| 121 | gactgcagtg taaatgtaga gaagcagccg ataaaatagc attgcctgaa gaagtttgga |
| 181 | ggctgagagc agcagtagac tggccaactg cagagcaagt tgtttctcca gccgtgcggt |
| 241 | gcagcctcat gcccccaacc cagcttagcc actgtaagaa gacgttcact gtacagacga |
| 301 | ccaaacttgc cgtggaagag acagttgtga gattcccttg caaatttaca tacgagaatg |
| 361 | gcttgtgaaa tcatgcctct gcaaagttca caggaagatg aaagacctct gtcacctttc |
| 421 | tatttgagtg ctcatgtacc ccaagtcagc aatgtgtctg caaccggaga actcttagaa |
| 481 | agaaccatcc gatcagctgt agaacaacat ctttttgatg ttaataactc tggaggtcaa |
| 541 | agttcagagg actcagaatc tggaacacta tcagcatctt ctgccacatc tgccagacag |
| 601 | cgccgccgcc agtccaagga gcaggatgaa gttcgacatg ggagagacaa gggacttatc |
| 661 | aacaaagaaa atactccttc tgggttcaac caccttgatg attgtatttt gaatactcag |
| 721 | gaagtcgaaa aggtacacaa aaatactttt ggttgtgctg gagaaaggag caagcctaaa |
| 781 | cgtcagaaat ccagtactaa actttctgag cttcatgaca atcaggacgg tcttgtgaat |
| 841 | atggaaagtc tcaattccac acgatctcat gagagaactg gacctgatga ttttgaatgg |
| 901 | atgtctgatg aaaggaaagg aaatgaaaaa gatggtggac acactcagca ttttgagagc |
| 961 | cccacaatga agatccagga gcatcccagc ctatctgaca ccaaacagca gagaaatcaa |
| 1021 | gatgccggtg accaggagga gagctttgtc tccgaagtgc cccagtcgga cctgactgca |
| 1081 | ttgtgtgatg aaaagaactg ggaagagcct atccctgctt tctcctcctg gcagcgggag |
| 1141 | aacagtgact ctgatgaagc ccacctctcg ccgcaggctg ggcgcctgat ccgtcagctg |
| 1201 | ctggacgaag acagcgaccc catgctctct cctcggttct acgcttatgg gcagagcagg |
| 1261 | caatacctgg atgacacaga agtgcctcct tccccaccaa actcccattc tttcatgagg |
| 1321 | cggcgaagct cctctctggg gtcctatgat gatgagcaag aggacctgac acctgcccag |
| 1381 | ctcacacgaa ggattcagag ccttaaaaag aagatccgga agtttgaaga tagattcgaa |
| 1441 | gaagagaaga agtacagacc ttcccacagt gacaaagcag ccaatccgga ggttctgaaa |
| 1501 | tggacaaatg accttgccaa attccggaga caacttaaag aatcaaaact aaagatatct |
| 1561 | gaagaggacc taactcccag gatgcggcag cgaagcaaca cactccccaa gagttttggt |
| 1621 | tcccaacttg agaaagaaga tgagaagaag caagagctgg tggataaagc aataaagccc |
| 1681 | agtgttgaag ccacattgga atctattcag aggaagctcc aggagaagcg agcggaaagc |
| 1741 | agccgccctg aggacattaa ggatatgacc aaagaccaga ttgctaatga gaaagtggct |
| 1801 | ctgcagaaag ctctgttata ttatgaaagc attcatggac ggccggtaac aaagaacgaa |
| 1861 | cggcaggtga tgaagccact atacgacagg taccggctgg tcaaacagat cctctcccga |
| 1921 | gctaacacca tacccatcat tgaagaagag gaggggtcag aagacgatag caatgtgaag |
| 1981 | ccagacttca tggtcactct gaaaaccgat ttcagtgcac gatgctttct ggaccaattc |
| 2041 | gaagatgacg ctgatggatt tatttcccca atggatgata aaataccatc aaaatgcagc |
| 2101 | caggacacag ggctttcaaa tctccatgct gcctcaatac ctgaactcct ggaacacctc |
| 2161 | caggaaatga gagaagaaaa gaaaaggatt cgaaagaaac ttcgggattt tgaagacaac |
| 2221 | tttttcagac agaatggaag aaatgtccag aaggaagacc gcactcctat ggctgaagaa |
| 2281 | tacagtgaat ataagcacat aaaggcgaaa ctgaggctcc tggaggtgct catcagcaag |
| 2341 | agagacactg attccaagtc catgtgaggg gcatggccaa gcacaggggg ctggcagctg |
| 2401 | cggtgagagt ttactgtccc cagagaaagt gcagctctgg aaggcagcct tggggctggc |
| 2461 | cctgcaaagc atgcagccct tctgcctcta gaccatttgg catcggctcc tgtttccatt |
| 2521 | gcctgcctta gaaactggct ggaagaagac aatgtgacct gacttaggca ttttgtaatt |
| 2581 | ggaaagtcaa gactgcagta tgtgcacatg cgcacgcgca tgcacgcaca cacacacaca |
| 2641 | gtagtggagc tttcctaaca ctagcagaga ttaatcacta cattagacaa cactcatcta |
| 2701 | cagagaatat acactgttct tccctggata actgagaaac aagagaccat tctctgtcta |
| 2761 | actgtgataa aaacaagctc aggactttat tctatagagc aaacttgctg tggagggcca |
| 2821 | tgctctcctt ggacccagtt aactgcaaac gtgcattgga gccctatttg ctgccgctgc |
| 2881 | cattctagtg acctttccac agagctgcgc cttcctcacg tgtgtgaaag gttttcccct |
| 2941 | tcagccctca ggtagatgga agctgcatct gcccacgatg gcagtgcagt catcatcttc |
| 3001 | aggatgtttc ttcaggactt cctcagctga caaggaattt tggtccctgc ctaggaccgg |
| 3061 | gtcatctgca gaggacagag agatggtaag cagctgtatg aatgctgatt ttaaaaccag |
| 3121 | gtcatgggag aagagcctgg agattctttc ctgaacactg actgcactta ccagtctgat |
| 3181 | tttatcgtca aacaccaagc caggctagca tgctcatggc aatctgtttg gggctgtttt |
| 3241 | gttgtggcac tagccaaaca taaaggggct taagtcagcc tgcatacaga ggatcgggga |
| 3301 | gagaaggggc ctgtgttctc agcctcctga gtacttacca gagtttaatt tttttaaaaa |
| 3361 | aaatctgcac taaaatcccc aaactgacag gtaaatgtag ccctcagagc tcagcccaag |
| 3421 | gcagaatcta aatcacacta ttttcgagat catgtataaa aagaaaaaaa agaagtcatg |
| 3481 | ctgtgtggcc aattataatt tttttcaaag actttgtcac aaaactgtct atattagaca |
| 3541 | ttttggaggg accaggaaat gtaagacacc aaatcctcca tctcttcagt gtgcctgatg |
| 3601 | tcacctcatg atttgctgtt acttttttaa ctcctgcgcc aaggacagtg ggttctgtgt |
| 3661 | ccacctttgt gctttgcgag gccgagccca ggcatctgct cgcctgccac ggctgaccag |
| 3721 | agaaggtgct tcaggagctc tgccttagac gacgtgttac agtatgaaca cacagcagag |
| 3781 | gcaccctcgt atgttttgaa agttgccttc tgaaagggca cagttttaag gaaaagaaaa |
| 3841 | agaatgtaaa actatactga cccgttttca gttttaaagg gtcgtgagaa actggctggt |
| 3901 | ccaatgggat ttacagcaac attttccatt gctgaagtga ggtagcagct ctcttctgtc |
| 3961 | agctgaatgt taaggatggg gaaaaagaat gcctttaagt ttgctcttaa tcgtatggaa |
| 4021 | gcttgagcta tgtgttggaa gtgccctggt tttaatccat acacaaagac ggtacataat |
| 4081 | cctacaggtt taaatgtaca taaaaatata gtttggaatt ctttgctcta ctgtttacat |
| 4141 | tgcagattgc tataatttca aggagtgaga ttataaataa aatgatgcac tttaggatgt |
| 4201 | ttcctatttt tgaaatctga acatgaatca ttcacatgac caaaaattgt gtttttttaa |
| 4261 | aaatacatgt ctagtctgtc ctttaatagc tctcttaaat aagctatgat attaatcaga |
| 4321 | tcattaccag ttagctttta aagcacattt gtttaagact atgtttttgg aaaaatacgc |
| 4381 | tacagaattt ttttttaagc tacaaataaa tgagatgcta ctaattgttt tggaatctgt |
| 4441 | tgtttctgcc aaaggtaaat taactaaaga tttattcagg aatccccatt tgaatttgta |
| 4501 | tgattcaata aaagaaaaca ccaagtaagt tatataaaat aaattgtgta tgagatgttg |
| 4561 | tgttttcctt tgtaatttcc actaactaac taactaactt atattcttca tggaatggag |
| 4621 | cccagaagaa atgagaggaa gcccttttca cactagatct tatttgaaga aatgtttgtt |
| 4681 | agtcagtcag tcagtggttt ctggctctgc cgagggagat gtgttcccca gcaaccattt |
| 4741 | ctgcagccca gaatctcaag gcactagagg cggtgtctta attaattggc ttcacaaaga |
| 4801 | caaaatgctc tggactggga tttttccttt gctgtgttgg gaatatgtgt ttattaatta |
| 4861 | gcacatgcca acaaaataaa tgtcaagagt tatttcataa gtgtaagtaa acttaagaat |
| 4921 | taaagagtgc agacttataa ttttca |

In some embodiments of the methods of the disclosure, the wild type human FAM13A gene of the disclosure consists of or comprises the amino acid sequence (Genbank Accession number: NP_001252508.1, transcript variant 4):

| (SEQ ID NO: 41) | |
|---|---|
| 1 | maceimplqs sqederplsp fylsahvpqv snvsatgell ertirsaveq hlfdvnnsgg |
| 61 | qssedsesgt lsassatsar qrrrqskeqd evrhgrdkgl inkentpsgf nhlddcilnt |
| 121 | qevekvhknt fgcagerskp krqksstkls elhdnqdglv nmeslnstrs hertgpddfe |
| 181 | wmsderkgne kdgghtqhfe sptmkigehp slsdtkqqrn qdagdqeesf vsevpqsdlt |
| 241 | alcdeknwee pipafsswqr ensdsdeahl spqagrlirq lldedsdpml sprfyaygqs |
| 301 | rqylddtevp psppnshsfm rrrssslgsy ddeqedltpa qltrriqslk kkirkfedrf |
| 361 | eeekkyrpsh sdkaanpevl kwtndlakfr rqlkesklki seedltprmr qrsntlpksf |
| 421 | gsqlekedek kqelvdkaik psveatlesi qrklqekrae ssrpedikdm tkdqianekv |
| 481 | alqkallyye sihgrpvtkn erqvmkplyd ryrlvkqils rantipiiee eegseddsnv |
| 541 | kpdfmvtlkt dfsarcfldq feddadgfis pmddkipskc sqdtglsnlh aasipelleh |
| 601 | lqemreekkr irkklrdfed nffrqngrnv qkedrtpmae eyseykhika klrllevlis |
| 661 | krdtdsksm |

In some embodiments of the methods of the disclosure, the wild type human FAM13A gene of the disclosure consists of or comprises the nucleic acid sequence (Genbank Accession number: NM_001265580.1, transcript variant 5):

| (SEQ ID NO: 42) | |
|---|---|
| 1 | attgaggagc agaaggagta gggtgcgggg gaggaggagg agcgccttta gtgctgcagc |
| 61 | agctgctgct ctgattggcc cggtggttca gctgcttccc tggaacaaaa ggtcaaagtg |
| 121 | gactgcagtg taaatgtaga gaagcagccg ataaaatagc attgcctgaa gaagtttgga |
| 181 | ggctgagagc agcagtagac tggccaactg cagagcaagt tgtttctcca gccgtgcggt |
| 241 | gcagcctcat gcccccaacc cagcttagcc actgtaagaa gacgttcact gtacagacga |
| 301 | ccaaacttgc cgtggaagag acagttgtga gattcccttg caaatttaca tacgagaatg |
| 361 | gcttgtgaaa tcatgcctct gcaaagactc ttagaaagaa ccatccgatc agctgtagaa |
| 421 | caacatcttt ttgatgttaa taactctgga ggtcaaagtt cagaggactc agaatctgga |
| 481 | acactatcag catcttctgc cacatctgcc agacagcgcc gccgccagtc caaggagcag |
| 541 | gatgaagttc gacatgggag agacaaggga cttatcaaca aagaaaatac tccttctggg |
| 601 | ttcaaccacc ttgatgattg tattttgaat actcaggaag tcgaaaaggt acacaaaaat |
| 661 | acttttggtt gtgctggaga aaggagcaag cctaaacgtc agaaatccag tactaaactt |
| 721 | tctgagcttc atgacaatca ggacggtctt gtgaatatgg aaagtctcaa ttccacacga |
| 781 | tctcatgaga gaactggacc tgatgatttt gaatggatgt ctgatgaaag gaaaggaaat |
| 841 | gaaaaagatg gtggacacac tcagcatttt gagagcccca caatgaagat ccaggagcat |
| 901 | cccagcctat ctgacaccaa acagcagaga aatcaagatg ccggtgacca ggaggagagc |
| 961 | tttgtctccg aagtgcccca gtcggacctg actgcattgt gtgatgaaaa gaactgggaa |
| 1021 | gagcctatcc ctgctttctc ctcctggcag cgggagaaca gtgactctga tgaagcccac |
| 1081 | ctctcgccgc aggctgggcg cctgatccgt cagctgctgg acgaagacag cgaccccatg |
| 1141 | ctctctcctc ggttctacgc ttatgggcag agcaggcaat acctggatga cacagaagtg |
| 1201 | cctccttccc caccaaactc ccattctttc atgaggcggc gaagctcctc tctggggtcc |
| 1261 | tatgatgatg agcaagagga cctgacacct gcccagctca cacgaaggat tcagagcctt |
| 1321 | aaaaagaaga tccggaagtt tgaagataga ttcgaagaag agaagaagta cagaccttcc |
| 1381 | cacagtgaca aagcagccaa tccggaggtt ctgaaatgga caaatgacct tgccaaattc |
| 1441 | cggagacaac ttaaagaatc aaaactaaag atatctgaag aggacctaac tcccaggatg |
| 1501 | cggcagcgaa gcaacacact ccccaagagt tttggttccc aacttgagaa agaagatgag |
| 1561 | aagaagcaag agctggtgga taaagcaata aagcccagtg ttgaagccac attggaatct |
| 1621 | attcagagga agctccagga gaagcgagcg gaaagcagcc gccctgagga cattaaggat |
| 1681 | atgaccaaag accagattgc taatgagaaa gtggctctgc agaaagctct gttatattat |
| 1741 | gaaagcattc atggacggcc ggtaacaaag aacgaacggc aggtgatgaa gccactatac |
| 1801 | gacaggtacc ggctggtcaa acagatcctc tcccgagcta acaccatacc catcattggt |
| 1861 | tccccctcca gcaagcggag aagccctttg ctgcagccaa ttatcgaggg cgaaactgct |
| 1921 | tccttcttca aggagataaa ggaagaagag gaggggtcag aagacgatag caatgtgaag |
| 1981 | ccagacttca tggtcactct gaaaaccgat ttcagtgcac gatgctttct ggaccaattc |
| 2041 | gaagatgacg ctgatggatt tatttcccca atggatgata aaataccatc aaaatgcagc |
| 2101 | caggacacag ggctttcaaa tctccatgct gcctcaatac ctgaactcct ggaacacctc |
| 2161 | caggaaatga gagaagaaaa gaaaaggatt cgaaagaaac ttcgggattt tgaagacaac |
| 2221 | tttttcagac agaatggaag aaatgtccag aaggaagacc gcactcctat ggctgaagaa |
| 2281 | tacagtgaat ataagcacat aaaggcgaaa ctgaggctcc tggaggtgct catcagcaag |
| 2341 | agagacactg attccaagtc catgtgaggg gcatggccaa gcacaggggg ctggcagctg |
| 2401 | cggtgagagt ttactgtccc cagagaaagt gcagctctgg aaggcagcct tggggctggc |
| 2461 | cctgcaaagc atgcagccct tctgcctcta gaccatttgg catcggctcc tgtttccatt |
| 2521 | gcctgcctta gaaactggct ggaagaagac aatgtgacct gacttaggca ttttgtaatt |
| 2581 | ggaaagtcaa gactgcagta tgtgcacatg cgcacgcgca tgcacgcaca cacacacaca |
| 2641 | gtagtggagc tttcctaaca ctagcagaga ttaatcacta cattagacaa cactcatcta |
| 2701 | cagagaatat acactgttct tccctggata actgagaaac aagagaccat tctctgtcta |
| 2761 | actgtgataa aaacaagctc aggactttat tctatagagc aaacttgctg tggagggcca |
| 2821 | tgctctcctt ggacccagtt aactgcaaac gtgcattgga gccctatttg ctgccgctgc |
| 2881 | cattctagtg acctttccac agagctgcgc cttcctcacg tgtgtgaaag gttttcccct |
| 2941 | tcagccctca ggtagatgga agctgcatct gcccacgatg gcagtgcagt catcatcttc |
| 3001 | aggatgtttc ttcaggactt cctcagctga caaggaattt tggtccctgc ctaggaccgg |
| 3061 | gtcatctgca gaggacagag agatggtaag cagctgtatg aatgctgatt ttaaaaccag |
| 3121 | gtcatgggag aagagcctgg agattctttc ctgaacactg actgcactta ccagtctgat |
| 3181 | tttatcgtca aacaccaagc caggctagca tgctcatggc aatctgtttg gggctgtttt |
| 3241 | gttgtggcac tagccaaaca taaaggggct taagtcagcc tgcatacaga ggatcgggga |
| 3301 | gagaaggggc ctgtgttctc agcctcctga gtacttacca gagtttaatt tttttaaaaa |
| 3361 | aaatctgcac taaaatcccc aaactgacag gtaaatgtag ccctcagagc tcagcccaag |
| 3421 | gcagaatcta aatcacacta ttttcgagat catgtataaa aagaaaaaaa agaagtcatg |
| 3481 | ctgtgtggcc aattataatt tttttcaaag actttgtcac aaaactgtct atattagaca |
| 3541 | ttttggaggg accaggaaat gtaagacacc aaatcctcca tctcttcagt gtgcctgatg |
| 3601 | tcacctcatg atttgctgtt acttttttaa ctcctgcgcc aaggacagtg ggttctgtgt |
| 3661 | ccacctttgt gctttgcgag gccgagccca ggcatctgct cgcctgccac ggctgaccag |
| 3721 | agaaggtgct tcaggagctc tgccttagac gacgtgttac agtatgaaca cacagcagag |
| 3781 | gcaccctcgt atgttttgaa agttgccttc tgaaagggca cagttttaag gaaaagaaaa |
| 3841 | agaatgtaaa actatactga cccgttttca gttttaaagg gtcgtgagaa actggctggt |
| 3901 | ccaatgggat ttacagcaac attttccatt gctgaagtga ggtagcagct ctcttctgtc |
| 3961 | agctgaatgt taaggatggg gaaaaagaat gcctttaagt ttgctcttaa tcgtatggaa |
| 4021 | gcttgagcta tgtgttggaa gtgccctggt tttaatccat acacaaagac ggtacataat |
| 4081 | cctacaggtt taaatgtaca taaaaatata gtttggaatt ctttgctcta ctgtttacat |
| 4141 | tgcagattgc tataatttca aggagtgaga ttataaataa aatgatgcac tttaggatgt |
| 4201 | ttcctatttt tgaaatctga acatgaatca ttcacatgac caaaaattgt gtttttttaa |
| 4261 | aaatacatgt ctagtctgtc ctttaatagc tctcttaaat aagctatgat attaatcaga |
| 4321 | tcattaccag ttagctttta aagcacattt gtttaagact atgtttttgg aaaaatacgc |
| 4381 | tacagaattt ttttttaagc tacaaataaa tgagatgcta ctaattgttt tggaatctgt |
| 4441 | tgtttctgcc aaaggtaaat taactaaaga tttattcagg aatccccatt tgaatttgta |
| 4501 | tgattcaata aaagaaaaca ccaagtaagt tatataaaat aaattgtgta tgagatgttg |
| 4561 | tgttttcctt tgtaatttcc actaactaac taactaactt atattcttca tggaatggag |
| 4621 | cccagaagaa atgagaggaa gcccttttca cactagatct tatttgaaga aatgtttgtt |
| 4681 | agtcagtcag tcagtggttt ctggctctgc cgagggagat gtgttcccca gcaaccattt |
| 4741 | ctgcagccca gaatctcaag gcactagagg cggtgtctta attaattggc ttcacaaaga |
| 4801 | caaaatgctc tggactggga tttttccttt gctgtgttgg gaatatgtgt ttattaatta |
| 4861 | gcacatgcca acaaaataaa tgtcaagagt tatttcataa gtgtaagtaa acttaagaat |
| 4921 | taaagagtgc agacttataa ttttca |

In some embodiments of the methods of the disclosure, the wild type human FAM13A gene of the disclosure consists of or comprises the amino acid sequence (Genbank Accession number: NP_001252509.1, transcript variant 5):

| (SEQ ID NO: 43) | |
|---|---|
| 1 | maceimplqr llertirsav eqhlfdvnns ggqssedses gtlsassats arqrrrqske |
| 61 | qdevrhgrdk glinkentps gfnhlddcil ntqevekvhk ntfgcagers kpkrqksstk |
| 121 | lselhdnqdg lvnmeslnst rshertgpdd fewmsderkg nekdgghtqh fesptmkiqe |
| 181 | hpslsdtkqq rnqdagdqee sfvsevpqsd ltalcdeknw eepipafssw qrensdsdea |
| 241 | hlspqagrli rqlldedsdp mlsprfyayg qsrqylddte vppsppnshs fmrrrssslg |
| 301 | syddeqedlt paqltrriqs lkkkirkfed rfeeekkyrp shsdkaanpe vlkwtndlak |
| 361 | frrqlkeskl kiseedltpr mrqrsntlpk sfgsqleked ekkqelvdka ikpsveatle |
| 421 | siqrklqekr aessrpedik dmtkdqiane kvalqkally yesihgrpvt knerqvmkpl |
| 481 | ydryrlvkqi lsrantipii gspsskrrsp llqpiieget asffkeikee eegseddsnv |
| 541 | kpdfmvtlkt dfsarcfldq feddadgfis pmddkipskc sqdtglsnlh aasipelleh |
| 601 | lqemreekkr irkklrdfed nffrqngrnv qkedrtpmae eyseykhika klrllevlis |
| 661 | krdtdsksm |

In some embodiments of the methods of the disclosure, the wild type human DSP gene of the disclosure consists of or comprises the nucleic acid sequence (Genbank Accession number: NM004415.3, transcript variant 1):

| (SEQ ID NO: 44) | |
|---|---|
| 1 | aagaaaccgg ccaggtgtgg cctaggcgcc cagtgccagc ggggaggaga ctcgctccgc |
| 61 | cgccgaccaa caccaacacc cagctccgac gcagctcctc tgcgcccttg ccgccctccg |
| 121 | agccacagct ttcctcccgc tcctgccccc ggcccgtcgc cgtctccgcg ctcgcagcgg |
| 181 | cctcgggagg gcccaggtag cgagcagcga cctcgcgagc cttccgcact cccgcccggt |
| 241 | tccccggccg tccgcctatc cttggccccc tccgctttct ccgcgccggc ccgcctcgct |
| 301 | tatgcctcgg cgctgagccg ctctcccgat tgcccgccga catgagctgc aacggaggct |
| 361 | cccacccgcg gatcaacact ctgggccgca tgatccgcgc cgagtctggc ccggacctgc |
| 421 | gctacgaggt gaccagcggc ggcgggggca ccagcaggat gtactattct cggcgcggcg |
| 481 | tgatcaccga ccagaactcg gacggctact gtcaaaccgg cacgatgtcc aggcaccaga |
| 541 | accagaacac catccaggag ctgctgcaga actgctccga ctgcttgatg cgagcagagc |
| 601 | tcatcgtgca gcctgaattg aagtatggag atggaataca actgactcgg agtcgagaat |
| 661 | tggatgagtg ttttgcccag gccaatgacc aaatggaaat cctcgacagc ttgatcagag |
| 721 | agatgcggca gatgggccag ccctgtgatg cttaccagaa aaggcttctt cagctccaag |
| 781 | agcaaatgcg agccctttat aaagccatca gtgtccctcg agtccgcagg gccagctcca |
| 841 | agggtggtgg aggctacact tgtcagagtg gctctggctg ggatgagttc accaaacatg |
| 901 | tcaccagtga atgtttgggg tggatgaggc agcaaagggc ggagatggac atggtggcct |
| 961 | ggggtgtgga cctggcctca gtggagcagc acattaacag ccaccggggc atccacaact |
| 1021 | ccatcggcga ctatcgctgg cagctggaca aaatcaaagc cgacctgcgc gagaaatctg |
| 1081 | cgatctacca gttggaggag gagtatgaaa acctgctgaa agcgtccttt gagaggatgg |
| 1141 | atcacctgcg acagctgcag aacatcattc aggccacgtc cagggagatc atgtggatca |
| 1201 | atgactgcga ggaggaggag ctgctgtacg actggagcga caagaacacc aacatcgctc |
| 1261 | agaaacagga ggccttctcc atacgcatga gtcaactgga agttaaagaa aaagagctca |
| 1321 | ataagctgaa acaagaaagt gaccaacttg tcctcaatca gcatccagct tcagacaaaa |
| 1381 | ttgaggccta tatggacact ctgcagacgc agtggagttg gattcttcag atcaccaagt |
| 1441 | gcattgatgt tcatctgaaa gaaaatgctg cctactttca gttttttgaa gaggcgcagt |
| 1501 | ctactgaagc atacctgaag gggctccagg actccatcag gaagaagtac ccctgcgaca |
| 1561 | agaacatgcc cctgcagcac ctgctggaac agatcaagga gctggagaaa gaacgagaga |
| 1621 | aaatccttga atacaagcgt caggtgcaga acttggtaaa caagtctaag aagattgtac |
| 1681 | agctgaagcc tcgtaaccca gactacagaa gcaataaacc cattattctc agagctctct |
| 1741 | gtgactacaa acaagatcag aaaatcgtgc ataaggggga tgagtgtatc ctgaaggaca |
| 1801 | acaacgagcg cagcaagtgg tacgtgacgg gcccgggagg cgttgacatg cttgttccct |
| 1861 | ctgtggggct gatcatccct cctccgaacc cactggccgt ggacctctct tgcaagattg |
| 1921 | agcagtacta cgaagccatc ttggctctgt ggaaccagct ctacatcaac atgaagagcc |
| 1981 | tggtgtcctg gcactactgc atgattgaca tagagaagat cagggccatg acaatcgcca |
| 2041 | agctgaaaac aatgcggcag gaagattaca tgaagacgat agccgacctt gagttacatt |
| 2101 | accaagagtt catcagaaat agccaaggct cagagatgtt tggagatgat gacaagcgga |
| 2161 | aaatacagtc tcagttcacc gatgcccaga agcattacca gaccctggtc attcagctcc |
| 2221 | ctggctatcc ccagcaccag acagtgacca caactgaaat cactcatcat ggaacctgcc |
| 2281 | aagatgtcaa ccataataaa gtaattgaaa ccaacagaga aaatgacaag caagaaacat |
| 2341 | ggatgctgat ggagctgcag aagattcgca ggcagataga gcactgcgag ggcaggatga |
| 2401 | ctctcaaaaa cctccctcta gcagaccagg gatcttctca ccacatcaca gtgaaaatta |
| 2461 | acgagcttaa gagtgtgcag aatgattcac aagcaattgc tgaggttctc aaccagctta |
| 2521 | aagatatgct tgccaacttc agaggttctg aaaagtactg ctatttacag aatgaagtat |
| 2581 | ttggactatt tcagaaactg gaaaatatca atggtgttac agatggctac ttaaatagct |
| 2641 | tatgcacagt aagggcactg ctccaggcta ttctccaaac agaagacatg ttaaaggttt |
| 2701 | atgaagccag gctcactgag gaggaaactg tctgcctgga cctggataaa gtggaagctt |
| 2761 | accgctgtgg actgaagaaa ataaaaaatg acttgaactt gaagaagtcg ttgttggcca |
| 2821 | ctatgaagac agaactacag aaagcccagc agatccactc tcagacttca cagcagtatc |
| 2881 | cactttatga tctggacttg ggcaagttcg gtgaaaaagt cacacagctg acagaccgct |
| 2941 | ggcaaaggat agataaacag atcgacttta ggttatggga cctggagaaa caaatcaagc |
| 3001 | aattgaggaa ttatcgtgat aactatcagg ctttctgcaa gtggctctat gatgctaaac |
| 3061 | gccgccagga ttccttagaa tccatgaaat ttggagattc caacacagtc atgcggtttt |
| 3121 | tgaatgagca gaagaacttg cacagtgaaa tatctggcaa acgagacaaa tcagaggaag |
| 3181 | tacaaaaaat tgctgaactt tgcgccaatt caattaagga ttatgagctc cagctggcct |
| 3241 | catacacctc aggactggaa actctgctga acatacctat caagaggacc atgattcagt |
| 3301 | ccccttctgg ggtgattctg caagaggctg cagatgttca tgctcggtac attgaactac |
| 3361 | ttacaagatc tggagactat tacaggttct taagtgagat gctgaagagt ttggaagatc |
| 3421 | tgaagctgaa aaataccaag atcgaagttt tggaagagga gctcagactg gcccgagatg |
| 3481 | ccaactcgga aaactgtaat aagaacaaat tcctggatca gaacctgcag aaataccagg |
| 3541 | cagagtgttc ccagttcaaa gcgaagcttg cgagcctgga ggagctgaag agacaggctg |
| 3601 | agctggatgg gaagtcggct aagcaaaatc tagacaagtg ctacggccaa ataaaagaac |
| 3661 | tcaatgagaa gatcacccga ctgacttatg agattgaaga tgaaaagaga agaagaaaat |
| 3721 | ctgtggaaga cagatttgac caacagaaga atgactatga ccaactgcag aaagcaaggc |
| 3781 | aatgtgaaaa ggagaacctt ggttggcaga aattagagtc tgagaaagcc atcaaggaga |
| 3841 | aggagtacga gattgaaagg ttgagggttc tactgcagga agaaggcacc cggaagagag |
| 3901 | aatatgaaaa tgagctggca aaggtaagaa accactataa tgaggagatg agtaatttaa |
| 3961 | ggaacaagta tgaaacagag attaacatta cgaagaccac catcaaggag atatccatgc |
| 4021 | aaaaagagga tgattccaaa aatcttagaa accagcttga tagactttca agggaaaatc |
| 4081 | gagatctgaa ggatgaaatt gtcaggctca atgacagcat cttgcaggcc actgagcagc |
| 4141 | gaaggcgagc tgaagaaaac gcccttcagc aaaaggcctg tggctctgag ataatgcaga |
| 4201 | agaagcagca tctggagata gaactgaagc aggtcatgca gcagcgctct gaggacaatg |
| 4261 | cccggcacaa gcagtccctg gaggaggctg ccaagaccat tcaggacaaa aataaggaga |
| 4321 | tcgagagact caaagctgag tttcaggagg aggccaagcg ccgctgggaa tatgaaaatg |
| 4381 | aactgagtaa ggtaagaaac aattatgatg aggagatcat tagcttaaaa aatcagtttg |
| 4441 | agaccgagat caacatcacc aagaccacca tccaccagct caccatgcag aaggaagagg |
| 4501 | ataccagtgg ctaccgggct cagatagaca atctcacccg agaaaacagg agcttatctg |
| 4561 | aagaaataaa gaggctgaag aacactctaa cccagaccac agagaatctc aggagggtgg |
| 4621 | aagaagacat ccaacagcaa aaggccactg gctctgaggt gtctcagagg aaacagcagc |
| 4681 | tggaggttga gctgagacaa gtcactcaga tgcgaacaga ggagagcgta agatataagc |
| 4741 | aatctcttga tgatgctgcc aaaaccatcc aggataaaaa caaggagata gaaaggttaa |
| 4801 | aacaactgat cgacaaagaa acaaatgacc ggaaatgcct ggaagatgaa aacgcgagat |
| 4861 | tacaaagggt ccagtatgac ctgcagaaag caaacagtag tgcgacggag acaataaaca |
| 4921 | aactgaaggt tcaggagcaa gaactgacac gcctgaggat cgactatgaa agggtttccc |
| 4981 | aggagaggac tgtgaaggac caggatatca cgcggttcca gaactctctg aaagagctgc |
| 5041 | agctgcagaa gcagaaggtg gaagaggagc tgaatcggct gaagaggacc gcgtcagaag |
| 5101 | actcctgcaa gaggaagaag ctggaggaag agctggaagg catgaggagg tcgctgaagg |
| 5161 | agcaagccat caaaatcacc aacctgaccc agcagctgga gcaggcatcc attgttaaga |
| 5221 | agaggagtga ggatgacctc cggcagcaga gggacgtgct ggatggccac ctgagggaaa |
| 5281 | agcagaggac ccaggaagag ctgaggaggc tctcttctga ggtcgaggcc ctgaggcggc |
| 5341 | agttactcca ggaacaggaa agtgtcaaac aagctcactt gaggaatgag catttccaga |
| 5401 | aggcgataga agataaaagc agaagcttaa atgaaagcaa aatagaaatt gagaggctgc |
| 5461 | agtctctcac agagaacctg accaaggagc acttgatgtt agaagaagaa ctgcggaacc |
| 5521 | tgaggctgga gtacgatgac ctgaggagag gacgaagcga agcggacagt gataaaaatg |
| 5581 | caaccatctt ggaactaagg agccagctgc agatcagcaa caaccggacc ctggaactgc |
| 5641 | aggggctgat taatgattta cagagagaga gggaaaattt gagacaggaa attgagaaat |
| 5701 | tccaaaagca ggctttagag gcatctaata ggattcagga atcaaagaat cagtgtactc |
| 5761 | aggtggtaca ggaaagagag agccttctgg tgaaaatcaa agtcctggag caagacaagg |
| 5821 | caaggctgca gaggctggag gatgagctga atcgtgcaaa atcaactcta gaggcagaaa |
| 5881 | ccagggtgaa acagcgcctg gagtgtgaga aacagcaaat tcagaatgac ctgaatcagt |
| 5941 | ggaagactca atattcccgc aaggaggagg ctattaggaa gatagaatcg gaaagagaaa |
| 6001 | agagtgagag agagaagaac agtcttagga gtgagatcga aagactccaa gcagagatca |
| 6061 | agagaattga agagaggtgc aggcgtaagc tggaggattc taccagggag acacagtcac |
| 6121 | agttagaaac agaacgctcc cgatatcaga gggagattga taaactcaga cagcgcccat |
| 6181 | atgggtccca tcgagagacc cagactgagt gtgagtggac cgttgacacc tccaagctgg |
| 6241 | tgtttgatgg gctgaggaag aaggtgacag caatgcagct ctatgagtgt cagctgatcg |
| 6301 | acaaaacaac cttggacaaa ctattgaagg ggaagaagtc agtggaagaa gttgcttctg |
| 6361 | aaatccagcc attccttcgg ggtgcaggat ctatcgctgg agcatctgct tctcctaagg |
| 6421 | aaaaatactc tttggtagag gccaagagaa agaaattaat cagcccagaa tccacagtca |
| 6481 | tgcttctgga ggcccaggca gctacaggtg gtataattga tccccatcgg aatgagaagc |
| 6541 | tgactgtcga cagtgccata gctcgggacc tcattgactt cgatgaccgt cagcagatat |
| 6601 | atgcagcaga aaaagctatc actggttttg atgatccatt ttcaggcaag acagtatctg |
| 6661 | tttcagaagc catcaagaaa aatttgattg atagagaaac cggaatgcgc ctgctggaag |
| 6721 | cccagattgc ttcagggggt gtagtagacc ctgtgaacag tgtctttttg ccaaaagatg |
| 6781 | tcgccttggc ccgggggctg attgatagag atttgtatcg atccctgaat gatccccgag |
| 6841 | atagtcagaa aaactttgtg gatccagtca ccaaaaagaa ggtcagttac gtgcagctga |
| 6901 | aggaacggtg cagaatcgaa ccacatactg gtctgctctt gctttcagta cagaagagaa |
| 6961 | gcatgtcctt ccaaggaatc agacaacctg tgaccgtcac tgagctagta gattctggta |
| 7021 | tattgagacc gtccactgtc aatgaactgg aatctggtca gatttcttat gacgaggttg |
| 7081 | gtgagagaat taaggacttc ctccagggtt caagctgcat agcaggcata tacaatgaga |
| 7141 | ccacaaaaca gaagcttggc atttatgagg ccatgaaaat tggcttagtc cgacctggta |
| 7201 | ctgctctgga gttgctggaa gcccaagcag ctactggctt tatagtggat cctgttagca |
| 7261 | acttgaggtt accagtggag gaagcctaca agagaggtct ggtgggcatt gagttcaaag |
| 7321 | agaagctcct gtctgcagaa cgagctgtca ctgggtataa tgatcctgaa acaggaaaca |
| 7381 | tcatctcttt gttccaagcc atgaataagg aactcatcga aaagggccac ggtattcgct |
| 7441 | tattagaagc acagatcgca accgggggga tcattgaccc aaaggagagc catcgtttac |
| 7501 | cagttgacat agcatataag aggggctatt tcaatgagga actcagtgag attctctcag |
| 7561 | atccaagtga tgataccaaa ggattttttg accccaacac tgaagaaaat cttacctatc |
| 7621 | tgcaactaaa agaaagatgc attaaggatg aggaaacagg gctctgtctt ctgcctctga |
| 7681 | aagaaaagaa gaaacaggtg cagacatcac aaaagaatac cctcaggaag cgtagagtgg |
| 7741 | tcatagttga cccagaaacc aataaagaaa tgtctgttca ggaggcctac aagaagggcc |
| 7801 | taattgatta tgaaaccttc aaagaactgt gtgagcagga atgtgaatgg gaagaaataa |
| 7861 | ccatcacggg atcagatggc tccaccaggg tggtcctggt agatagaaag acaggcagtc |
| 7921 | agtatgatat tcaagatgct attgacaagg gccttgttga caggaagttc tttgatcagt |
| 7981 | accgatccgg cagcctcagc ctcactcaat ttgctgacat gatctccttg aaaaatggtg |
| 8041 | tcggcaccag cagcagcatg ggcagtggtg tcagcgatga tgtttttagc agctcccgac |
| 8101 | atgaatcagt aagtaagatt tccaccatat ccagcgtcag gaatttaacc ataaggagca |
| 8161 | gctctttttc agacaccctg gaagaatcga gccccattgc agccatcttt gacacagaaa |
| 8221 | acctggagaa aatctccatt acagaaggta tagagcgggg catcgttgac agcatcacgg |
| 8281 | gtcagaggct tctggaggct caggcctgca caggtggcat catccaccca accacgggcc |
| 8341 | agaagctgtc acttcaggac gcagtctccc agggtgtgat tgaccaagac atggccacca |
| 8401 | ggctgaagcc tgctcagaaa gccttcatag gcttcgaggg tgtgaaggga aagaagaaga |
| 8461 | tgtcagcagc agaggcagtg aaagaaaaat ggctcccgta tgaggctggc cagcgcttcc |
| 8521 | tggagttcca gtacctcacg ggaggtcttg ttgacccgga agtgcatggg aggataagca |
| 8581 | ccgaagaagc catccggaag gggttcatag atggccgcgc cgcacagagg ctgcaagaca |
| 8641 | ccagcagcta tgccaaaatc ctgacctgcc ccaaaaccaa attaaaaata tcctataagg |
| 8701 | atgccataaa tcgctccatg gtagaagata tcactgggct gcgccttctg gaagccgcct |
| 8761 | ccgtgtcgtc caagggctta cccagccctt acaacatgtc ttcggctccg gggtcccgct |
| 8821 | ccggctcccg ctcgggatct cgctccggat ctcgctccgg gtcccgcagt gggtcccgga |
| 8881 | gaggaagctt tgacgccaca gggaattctt cctactctta ttcctactca tttagcagta |
| 8941 | gttctattgg gcactagtag tcagttggga gtggttgcta taccttgact tcatttatat |
| 9001 | gaatttccac tttattaaat aatagaaaag aaaatcccgg tgcttgcagt agagtgatag |
| 9061 | gacattctat gcttacagaa aatatagcca tgattgaaat caaatagtaa aggctgttct |
| 9121 | ggctttttat cttcttagct catcttaaat aagcagtaca cttggatgca gtgcgtctga |
| 9181 | agtgctaatc agttgtaaca atagcacaaa tcgaacttag gatttgtttc ttctcttctg |
| 9241 | tgtttcgatt tttgatcaat tctttaattt tggaagccta taatacagtt ttctattctt |
| 9301 | ggagataaaa attaaatgga tcactgatat tttagtcatt ctgcttctca tctaaatatt |
| 9361 | tccatattct gtattaggag aaaattaccc tcccagcacc agcccccctc tcaaaccccc |
| 9421 | aacccaaaac caagcatttt ggaatgagtc tcctttagtt tcagagtgtg gattgtataa |
| 9481 | cccatatact cttcgatgta cttgtttggt ttggtattaa tttgactgtg catgacagcg |
| 9541 | gcaatctttt ctttggtcaa agttttctgt ttattttgct tgtcatattc gatgtacttt |
| 9601 | aaggtgtctt tatgaagttt gctattctgg caataaactt ttagactttt gaagtgtttg |
| 9661 | tgttttaatt taatatgttt ataagcatgt ataaacattt agcatatttt tatcataggt |
| 9721 | ctaaaaatat ttgtttacta aatacctgtg aagaaatacc attaaaaaac tatttggttc |
| 9781 | tgaattctta ctagaaaaaa aa |

In some embodiments of the methods of the disclosure, the wild type human DSP gene of the disclosure consists of or comprises the amino acid sequence (Genbank Accession number: NP 004406.2, transcript variant 1):

| (SEQ ID NO: 45) | |
|---|---|
| 1 | mscnggshpr intlgrmira esgpdlryev tsggggtsrm yysrrgvitd qnsdgycqtg |
| 61 | tmsrhqnqnt iqellqncsd clmraelivq pelkygdgiq ltrsreldec faqandqmei |
| 121 | ldsliremrq mgqpcdayqk rllqlqeqmr alykaisvpr vrrasskggg gytcqsgsgw |
| 181 | deftkhvtse clgwmrqqra emdmvawgvd lasveqhins hrgihnsigd yrwqldkika |
| 241 | dlreksaiyq leeeyenllk asfermdhlr qlqniiqats reimwindce eeellydwsd |
| 301 | kntniaqkqe afsirmsqle vkekelnklk qesdqlvinq hpasdkieay mdtlqtqwsw |
| 361 | ilqitkcidv hlkenaayfq ffeeaqstea ylkglqdsir kkypcdknmp lqhlleqike |
| 421 | lekerekile ykrqvqnlvn kskkivqlkp rnpdyrsnkp iilralcdyk qdqkivhkgd |
| 481 | ecilkdnner skwyvtgpgg vdmlvpsvgl iipppnplav dlsckieqyy eailalwnql |
| 541 | yinmkslvsw hycmidieki ramtiaklkt mrqedymkti adlelhyqef irnsqgsemf |
| 601 | gdddkrkiqs qftdaqkhyq tlviqlpgyp qhqtvtttei thhgtcqdvn hnkvietnre |
| 661 | ndkqetwmlm elqkirrqie hcegrmtlkn lpladqgssh hitvkinelk svqndsgqia |
| 721 | evlnqlkdml anfrgsekyc ylqnevfglf qkleningvt dgylnslctv rallqailqt |
| 781 | edmlkvyear lteeetvcld ldkveayrcg lkkikndlnl kksllatmkt elqkaqqihs |
| 841 | qtsqqyplyd ldlgkfgekv tqltdrwqri dkqidfrlwd lekqikqlrn yrdnyqafck |
| 901 | wlydakrrqd slesmkfgds ntvmrflneq knlhseisgk rdkseevqki aelcansikd |
| 961 | yelqlasyts gletllnipi krtmiqspsg vilqeaadvh aryielltrs gdyyrflsem |
| 1021 | lksledlklk ntkievleee lrlardanse ncnknkfldq nlqkyqaecs qfkaklasle |
| 1081 | elkrqaeldg ksakqnldkc ygqikelnek itrltyeied ekrrrksved rfdqqkndyd |
| 1141 | qlqkarqcek enlgwqkles ekaikekeye ierlrvllqe egtrkreyen elakvrnhyn |
| 1201 | eemsnlrnky eteinitktt ikeismqked dsknlrnqld rlsrenrdlk deivrlndsi |
| 1261 | lqateqrrra eenalqqkac gseimqkkqh leielkqvmq qrsednarhk qsleeaakti |
| 1321 | qdknkeierl kaefqeeakr rweyenelsk vrnnydeeii slknqfetei nitkttihql |
| 1381 | tmqkeedtsg yraqidnltr enrslseeik rlkntltqtt enlrrveedi qqqkatgsev |
| 1441 | sqrkqqleve lrqvtqmrte esvrykqsld daaktiqdkn keierlkqli dketndrkcl |
| 1501 | edenarlqry qydlqkanss atetinklkv qeqeltrlri dyervsgert vkdqditrfq |
| 1561 | nslkelqlqk qkveeelnrl krtasedsck rkkleeeleg mrrslkeqai kitnitqqle |
| 1621 | qasivkkrse ddlrqqrdvl dghlrekqrt qeelrrlsse vealrrqllq eqesvkqahl |
| 1681 | rnehfqkaie dksrslnesk ieierlqslt enitkehlml eeelrnlrle yddlrrgrse |
| 1741 | adsdknatil elrsqlqisn nrtlelqgli ndlqrerenl rqeiekfqkq aleasnriqe |
| 1801 | sknqctqvvq eresllvkik vleqdkarlq rledelnrak stleaetrvk qrlecekqqi |
| 1861 | qndlnqwktq ysrkeeairk ieserekser eknslrseie rlqaeikrie ercrrkleds |
| 1921 | tretqsqlet ersrygreid klrqrpygsh retqtecewt vdtsklvfdg lrkkvtamql |
| 1981 | yecqlidktt ldkllkgkks veevaseiqp flrgagsiag asaspkekys lveakrkkli |
| 2041 | spestvmlle aqaatggiid phrnekltvd saiardlidf ddrqqiyaae kaitgfddpf |
| 2101 | sgktvsvsea ikknlidret gmrlleaqia sggvvdpvns vflpkdvala rglidrdlyr |
| 2161 | slndprdsqk nfvdpvtkkk vsyvqlkerc riephtglll lsvqkrsmsf qgirqpvtvt |
| 2221 | elvdsgilrp stvnelesgq isydevgeri kdflqgssci agiynettkq klgiyeamki |
| 2281 | glvrpgtale lleaqaatgf ivdpvsnlrl pveeaykrgl vgiefkekll saeravtgyn |
| 2341 | dpetgniisl fqamnkelie kghgirllea qiatggiidp keshrlpvdi aykrgyfnee |
| 2401 | lseilsdpsd dtkgffdpnt eenltylqlk ercikdeetg lcllplkekk kqvqtsqknt |
| 2461 | lrkrrvvivd petnkemsvq eaykkglidy etfkelceqe ceweeititg sdgstrvvlv |
| 2521 | drktgsqydi qdaidkglvd rkffdqyrsg slsltqfadm islkngvgts ssmgsgvsdd |
| 2581 | vfsssrhesv skistissvr nltirsssfs dtleesspia aifdtenlek isitegierg |
| 2641 | ivdsitgqrl leaqactggi ihpttgqkls lqdaysqgvi dqdmatrlkp aqkafigfeg |
| 2701 | vkgkkkmsaa eavkekwlpy eagqrflefq yltgglvdpe vhgristeea irkgfidgra |
| 2761 | aqrlqdtssy akiltcpktk lkisykdain rsmveditgl rlleaasvss kglpspynms |
| 2821 | sapgsrsgsr sgsrsgsrsg srsgsrrgsf datgnssysy sysfssssig h |

In some embodiments of the methods of the disclosure, the wild type human DSP gene of the disclosure consists of or comprises the nucleic acid sequence (Genbank Accession number: NM_001008844.2, transcript variant 2):

| (SEQ ID NO: 19) | |
|---|---|
| 1 | aagaaaccgg ccaggtgtgg cctaggcgcc cagtgccagc ggggaggaga ctcgctccgc |
| 61 | cgccgaccaa caccaacacc cagctccgac gcagctcctc tgcgcccttg ccgccctccg |
| 121 | agccacagct ttcctcccgc tcctgccccc ggcccgtcgc cgtctccgcg ctcgcagcgg |
| 181 | cctcgggagg gcccaggtag cgagcagcga cctcgcgagc cttccgcact cccgcccggt |
| 241 | tccccggccg tccgcctatc cttggccccc tccgctttct ccgcgccggc ccgcctcgct |
| 301 | tatgcctcgg cgctgagccg ctctcccgat tgcccgccga catgagctgc aacggaggct |
| 361 | cccacccgcg gatcaacact ctgggccgca tgatccgcgc cgagtctggc ccggacctgc |
| 421 | gctacgaggt gaccagcggc ggcgggggca ccagcaggat gtactattct cggcgcggcg |
| 481 | tgatcaccga ccagaactcg gacggctact gtcaaaccgg cacgatgtcc aggcaccaga |
| 541 | accagaacac catccaggag ctgctgcaga actgctccga ctgcttgatg cgagcagagc |
| 601 | tcatcgtgca gcctgaattg aagtatggag atggaataca actgactcgg agtcgagaat |
| 661 | tggatgagtg ttttgcccag gccaatgacc aaatggaaat cctcgacagc ttgatcagag |
| 721 | agatgcggca gatgggccag ccctgtgatg cttaccagaa aaggcttctt cagctccaag |
| 781 | agcaaatgcg agccctttat aaagccatca gtgtccctcg agtccgcagg gccagctcca |
| 841 | agggtggtgg aggctacact tgtcagagtg gctctggctg ggatgagttc accaaacatg |
| 901 | tcaccagtga atgtttgggg tggatgaggc agcaaagggc ggagatggac atggtggcct |
| 961 | ggggtgtgga cctggcctca gtggagcagc acattaacag ccaccggggc atccacaact |
| 1021 | ccatcggcga ctatcgctgg cagctggaca aaatcaaagc cgacctgcgc gagaaatctg |
| 1081 | cgatctacca gttggaggag gagtatgaaa acctgctgaa agcgtccttt gagaggatgg |
| 1141 | atcacctgcg acagctgcag aacatcattc aggccacgtc cagggagatc atgtggatca |
| 1201 | atgactgcga ggaggaggag ctgctgtacg actggagcga caagaacacc aacatcgctc |
| 1261 | agaaacagga ggccttctcc atacgcatga gtcaactgga agttaaagaa aaagagctca |
| 1321 | ataagctgaa acaagaaagt gaccaacttg tcctcaatca gcatccagct tcagacaaaa |
| 1381 | ttgaggccta tatggacact ctgcagacgc agtggagttg gattcttcag atcaccaagt |
| 1441 | gcattgatgt tcatctgaaa gaaaatgctg cctactttca gttttttgaa gaggcgcagt |
| 1501 | ctactgaagc atacctgaag gggctccagg actccatcag gaagaagtac ccctgcgaca |
| 1561 | agaacatgcc cctgcagcac ctgctggaac agatcaagga gctggagaaa gaacgagaga |
| 1621 | aaatccttga atacaagcgt caggtgcaga acttggtaaa caagtctaag aagattgtac |
| 1681 | agctgaagcc tcgtaaccca gactacagaa gcaataaacc cattattctc agagctctct |
| 1741 | gtgactacaa acaagatcag aaaatcgtgc ataaggggga tgagtgtatc ctgaaggaca |
| 1801 | acaacgagcg cagcaagtgg tacgtgacgg gcccgggagg cgttgacatg cttgttccct |
| 1861 | ctgtggggct gatcatccct cctccgaacc cactggccgt ggacctctct tgcaagattg |
| 1921 | agcagtacta cgaagccatc ttggctctgt ggaaccagct ctacatcaac atgaagagcc |
| 1981 | tggtgtcctg gcactactgc atgattgaca tagagaagat cagggccatg acaatcgcca |
| 2041 | agctgaaaac aatgcggcag gaagattaca tgaagacgat agccgacctt gagttacatt |
| 2101 | accaagagtt catcagaaat agccaaggct cagagatgtt tggagatgat gacaagcgga |
| 2161 | aaatacagtc tcagttcacc gatgcccaga agcattacca gaccctggtc attcagctcc |
| 2221 | ctggctatcc ccagcaccag acagtgacca caactgaaat cactcatcat ggaacctgcc |
| 2281 | aagatgtcaa ccataataaa gtaattgaaa ccaacagaga aaatgacaag caagaaacat |
| 2341 | ggatgctgat ggagctgcag aagattcgca ggcagataga gcactgcgag ggcaggatga |
| 2401 | ctctcaaaaa cctccctcta gcagaccagg gatcttctca ccacatcaca gtgaaaatta |
| 2461 | acgagcttaa gagtgtgcag aatgattcac aagcaattgc tgaggttctc aaccagctta |
| 2521 | aagatatgct tgccaacttc agaggttctg aaaagtactg ctatttacag aatgaagtat |
| 2581 | ttggactatt tcagaaactg gaaaatatca atggtgttac agatggctac ttaaatagct |
| 2641 | tatgcacagt aagggcactg ctccaggcta ttctccaaac agaagacatg ttaaaggttt |
| 2701 | atgaagccag gctcactgag gaggaaactg tctgcctgga cctggataaa gtggaagctt |
| 2761 | accgctgtgg actgaagaaa ataaaaaatg acttgaactt gaagaagtcg ttgttggcca |
| 2821 | ctatgaagac agaactacag aaagcccagc agatccactc tcagacttca cagcagtatc |
| 2881 | cactttatga tctggacttg ggcaagttcg gtgaaaaagt cacacagctg acagaccgct |
| 2941 | ggcaaaggat agataaacag atcgacttta ggttatggga cctggagaaa caaatcaagc |
| 3001 | aattgaggaa ttatcgtgat aactatcagg ctttctgcaa gtggctctat gatgctaaac |
| 3061 | gccgccagga ttccttagaa tccatgaaat ttggagattc caacacagtc atgcggtttt |
| 3121 | tgaatgagca gaagaacttg cacagtgaaa tatctggcaa acgagacaaa tcagaggaag |
| 3181 | tacaaaaaat tgctgaactt tgcgccaatt caattaagga ttatgagctc cagctggcct |
| 3241 | catacacctc aggactggaa actctgctga acatacctat caagaggacc atgattcagt |
| 3301 | ccccttctgg ggtgattctg caagaggctg cagatgttca tgctcggtac attgaactac |
| 3361 | ttacaagatc tggagactat tacaggttct taagtgagat gctgaagagt ttggaagatc |
| 3421 | tgaagctgaa aaataccaag atcgaagttt tggaagagga gctcagactg gcccgagatg |
| 3481 | ccaactcgga aaactgtaat aagaacaaat tcctggatca gaacctgcag aaataccagg |
| 3541 | cagagtgttc ccagttcaaa gcgaagcttg cgagcctgga ggagctgaag agacaggctg |
| 3601 | agctggatgg gaagtcggct aagcaaaatc tagacaagtg ctacggccaa ataaaagaac |
| 3661 | tcaatgagaa gatcacccga ctgacttatg agattgaaga tgaaaagaga agaagaaaat |
| 3721 | ctgtggaaga cagatttgac caacagaaga atgactatga ccaactgcag aaagcaaggc |
| 3781 | aatgtgaaaa ggagaacctt ggttggcaga aattagagtc tgagaaagcc atcaaggaga |
| 3841 | aggagtacga gattgaaagg ttgagggttc tactgcagga agaaggcacc cggaagagag |
| 3901 | aatatgaaaa tgagctggca aaggcatcta ataggattca ggaatcaaag aatcagtgta |
| 3961 | ctcaggtggt acaggaaaga gagagccttc tggtgaaaat caaagtcctg gagcaagaca |
| 4021 | aggcaaggct gcagaggctg gaggatgagc tgaatcgtgc aaaatcaact ctagaggcag |
| 4081 | aaaccagggt gaaacagcgc ctggagtgtg agaaacagca aattcagaat gacctgaatc |
| 4141 | agtggaagac tcaatattcc cgcaaggagg aggctattag gaagatagaa tcggaaagag |
| 4201 | aaaagagtga gagagagaag aacagtctta ggagtgagat cgaaagactc caagcagaga |
| 4261 | tcaagagaat tgaagagagg tgcaggcgta agctggagga ttctaccagg gagacacagt |
| 4321 | cacagttaga aacagaacgc tcccgatatc agagggagat tgataaactc agacagcgcc |
| 4381 | catatgggtc ccatcgagag acccagactg agtgtgagtg gaccgttgac acctccaagc |
| 4441 | tggtgtttga tgggctgagg aagaaggtga cagcaatgca gctctatgag tgtcagctga |
| 4501 | tcgacaaaac aaccttggac aaactattga aggggaagaa gtcagtggaa gaagttgctt |
| 4561 | ctgaaatcca gccattcctt cggggtgcag gatctatcgc tggagcatct gcttctccta |
| 4621 | aggaaaaata ctctttggta gaggccaaga gaaagaaatt aatcagccca gaatccacag |
| 4681 | tcatgcttct ggaggcccag gcagctacag gtggtataat tgatccccat cggaatgaga |
| 4741 | agctgactgt cgacagtgcc atagctcggg acctcattga cttcgatgac cgtcagcaga |
| 4801 | tatatgcagc agaaaaagct atcactggtt ttgatgatcc attttcaggc aagacagtat |
| 4861 | ctgtttcaga agccatcaag aaaaatttga ttgatagaga aaccggaatg cgcctgctgg |
| 4921 | aagcccagat tgcttcaggg ggtgtagtag accctgtgaa cagtgtcttt ttgccaaaag |
| 4981 | atgtcgcctt ggcccggggg ctgattgata gagatttgta tcgatccctg aatgatcccc |
| 5041 | gagatagtca gaaaaacttt gtggatccag tcaccaaaaa gaaggtcagt tacgtgcagc |
| 5101 | tgaaggaacg gtgcagaatc gaaccacata ctggtctgct cttgctttca gtacagaaga |
| 5161 | gaagcatgtc cttccaagga atcagacaac ctgtgaccgt cactgagcta gtagattctg |
| 5221 | gtatattgag accgtccact gtcaatgaac tggaatctgg tcagatttct tatgacgagg |
| 5281 | ttggtgagag aattaaggac ttcctccagg gttcaagctg catagcaggc atatacaatg |
| 5341 | agaccacaaa acagaagctt ggcatttatg aggccatgaa aattggctta gtccgacctg |
| 5401 | gtactgctct ggagttgctg gaagcccaag cagctactgg ctttatagtg gatcctgtta |
| 5461 | gcaacttgag gttaccagtg gaggaagcct acaagagagg tctggtgggc attgagttca |
| 5521 | aagagaagct cctgtctgca gaacgagctg tcactgggta taatgatcct gaaacaggaa |
| 5581 | acatcatctc tttgttccaa gccatgaata aggaactcat cgaaaagggc cacggtattc |
| 5641 | gcttattaga agcacagatc gcaaccgggg ggatcattga cccaaaggag agccatcgtt |
| 5701 | taccagttga catagcatat aagaggggct atttcaatga ggaactcagt gagattctct |
| 5761 | cagatccaag tgatgatacc aaaggatttt ttgaccccaa cactgaagaa aatcttacct |
| 5821 | atctgcaact aaaagaaaga tgcattaagg atgaggaaac agggctctgt cttctgcctc |
| 5881 | tgaaagaaaa gaagaaacag gtgcagacat cacaaaagaa taccctcagg aagcgtagag |
| 5941 | tggtcatagt tgacccagaa accaataaag aaatgtctgt tcaggaggcc tacaagaagg |
| 6001 | gcctaattga ttatgaaacc ttcaaagaac tgtgtgagca ggaatgtgaa tgggaagaaa |
| 6061 | taaccatcac gggatcagat ggctccacca gggtggtcct ggtagataga aagacaggca |
| 6121 | gtcagtatga tattcaagat gctattgaca agggccttgt tgacaggaag ttctttgatc |
| 6181 | agtaccgatc cggcagcctc agcctcactc aatttgctga catgatctcc ttgaaaaatg |
| 6241 | gtgtcggcac cagcagcagc atgggcagtg gtgtcagcga tgatgttttt agcagctccc |
| 6301 | gacatgaatc agtaagtaag atttccacca tatccagcgt caggaattta accataagga |
| 6361 | gcagctcttt ttcagacacc ctggaagaat cgagccccat tgcagccatc tttgacacag |
| 6421 | aaaacctgga gaaaatctcc attacagaag gtatagagcg gggcatcgtt gacagcatca |
| 6481 | cgggtcagag gcttctggag gctcaggcct gcacaggtgg catcatccac ccaaccacgg |
| 6541 | gccagaagct gtcacttcag gacgcagtct cccagggtgt gattgaccaa gacatggcca |
| 6601 | ccaggctgaa gcctgctcag aaagccttca taggcttcga gggtgtgaag ggaaagaaga |
| 6661 | agatgtcagc agcagaggca gtgaaagaaa aatggctccc gtatgaggct ggccagcgct |
| 6721 | tcctggagtt ccagtacctc acgggaggtc ttgttgaccc ggaagtgcat gggaggataa |
| 6781 | gcaccgaaga agccatccgg aaggggttca tagatggccg cgccgcacag aggctgcaag |
| 6841 | acaccagcag ctatgccaaa atcctgacct gccccaaaac caaattaaaa atatcctata |
| 6901 | aggatgccat aaatcgctcc atggtagaag atatcactgg gctgcgcctt ctggaagccg |
| 6961 | cctccgtgtc gtccaagggc ttacccagcc cttacaacat gtcttcggct ccggggtccc |
| 7021 | gctccggctc ccgctcggga tctcgctccg gatctcgctc cgggtcccgc agtgggtccc |
| 7081 | ggagaggaag ctttgacgcc acagggaatt cttcctactc ttattcctac tcatttagca |
| 7141 | gtagttctat tgggcactag tagtcagttg ggagtggttg ctataccttg acttcattta |
| 7201 | tatgaatttc cactttatta aataatagaa aagaaaatcc cggtgcttgc agtagagtga |
| 7261 | taggacattc tatgcttaca gaaaatatag ccatgattga aatcaaatag taaaggctgt |
| 7321 | tctggctttt tatcttctta gctcatctta aataagcagt acacttggat gcagtgcgtc |
| 7381 | tgaagtgcta atcagttgta acaatagcac aaatcgaact taggatttgt ttcttctctt |
| 7441 | ctgtgtttcg atttttgatc aattctttaa ttttggaagc ctataataca gttttctatt |
| 7501 | cttggagata aaaattaaat ggatcactga tattttagtc attctgcttc tcatctaaat |
| 7561 | atttccatat tctgtattag gagaaaatta ccctcccagc accagccccc ctctcaaacc |
| 7621 | cccaacccaa aaccaagcat tttggaatga gtctccttta gtttcagagt gtggattgta |
| 7681 | taacccatat actcttcgat gtacttgttt ggtttggtat taatttgact gtgcatgaca |
| 7741 | gcggcaatct tttctttggt caaagttttc tgtttatttt gcttgtcata ttcgatgtac |
| 7801 | tttaaggtgt ctttatgaag tttgctattc tggcaataaa cttttagact tttgaagtgt |
| 7861 | ttgtgtttta atttaatatg tttataagca tgtataaaca tttagcatat ttttatcata |
| 7921 | ggtctaaaaa tatttgttta ctaaatacct gtgaagaaat accattaaaa aactatttgg |
| 7981 | ttctgaattc ttactagaaa aaaaa |

In some embodiments of the methods of the disclosure, the wild type human DSP gene of the disclosure consists of or comprises the amino acid sequence (Genbank Accession number: NP_001008844.1, transcript variant 2):

| (SEQ ID NO: 20) | |
|---|---|
| 1 | mscnggshpr intlgrmira esgpdlryev tsggggtsrm yysrrgvitd qnsdgycqtg |
| 61 | tmsrhqnqnt iqellqncsd clmraelivq pelkygdgiq ltrsreldec faqandqmei |
| 121 | ldsliremrq mgqpcdayqk rllqlqeqmr alykaisvpr vrrasskggg gytcqsgsgw |
| 181 | deftkhvtse clgwmrqqra emdmvawgvd lasveqhins hrgihnsigd yrwqldkika |
| 241 | dlreksaiyq leeeyenllk asfermdhlr qlqniiqats reimwindce eeellydwsd |
| 301 | kntniaqkqe afsirmsqle vkekelnklk qesdqlvinq hpasdkieay mdtlqtqwsw |
| 361 | ilqitkcidv hlkenaayfq ffeeaqstea ylkglqdsir kkypcdknmp lqhlleqike |
| 421 | lekerekile ykrqvqnlvn kskkivqlkp rnpdyrsnkp iilralcdyk qdqkivhkgd |
| 481 | ecilkdnner skwyvtgpgg vdmlvpsvgl iipppnplav dlsckieqyy eailalwnql |
| 541 | yinmkslvsw hycmidieki ramtiaklkt mrqedymkti adlelhyqef irnsqgsemf |
| 601 | gdddkrkiqs qftdaqkhyq tiviqlpgyp qhqtvtttei thhgtcqdvn hnkvietnre |
| 661 | ndkqetwmlm elqkirrqie hcegrmtlkn lpladqgssh hitvkinelk svqndsgaia |
| 721 | evinqlkdml anfrgsekyc ylqnevfglf qkleningvt dgylnslctv rallqailqt |
| 781 | edmlkvyear lteeetvcld ldkveayrcg lkkikndlnl kksllatmkt elqkaqqihs |
| 841 | qtsqqyplyd ldlgkfgekv tqltdrwqri dkqidfrlwd lekqikqlrn yrdnyqafck |
| 901 | wlydakrrqd slesmkfgds ntvmrflneq knlhseisgk rdkseevqki aelcansikd |
| 961 | yelqlasyts gletllnipi krtmiqspsg vilqeaadvh aryielltrs gdyyrflsem |
| 1021 | lksledlklk ntkievleee lrlardanse ncnknkfldq nlqkyqaecs qfkaklasle |
| 1081 | elkrqaeldg ksakqnldkc ygqikelnek itrltyeied ekrrrksved rfdqqkndyd |
| 1141 | qlqkarqcek enlgwqkles ekaikekeye ierlrvllqe egtrkreyen elakasnriq |
| 1201 | esknqctqvv qeresllvki kvleqdkarl qrledelnra kstleaetrv kqrlecekqq |
| 1261 | iqndlnqwkt qysrkeeair kieserekse reknslrsei erlqaeikri eercrrkled |
| 1321 | stretqsqle tersrygrei dklrqrpygs hretqtecew tvdtsklvfd glrkkvtamq |
| 1381 | lyecqlidkt tldkllkgkk sveevaseiq pflrgagsia gasaspkeky slveakrkkl |
| 1441 | ispestvmll eaqaatggii dphrnekltv dsaiardlid fddrqqiyaa ekaitgfddp |
| 1501 | fsgktvsvse aikknlidre tgmrlleaqi asggvvdpvn svflpkdval arglidrdly |
| 1561 | rslndprdsq knfvdpvtkk kvsyvqlker criephtgll llsvqkrsms fqgirqpvtv |
| 1621 | telvdsgilr pstvnelesg qisydevger ikdflqgssc iagiynettk qklgiyeamk |
| 1681 | iglvrpgtal elleaqaatg fivdpvsnlr lpveeaykrg lvgiefkekl lsaeravtgy |
| 1741 | ndpetgniis lfqamnkeli ekghgirlle aqiatggiid pkeshrlpvd iaykrgyfne |
| 1801 | elseilsdps ddtkgffdpn teenitylql kercikdeet glcllplkek kkqvqtsqkn |
| 1861 | tlrkrrvviv dpetnkemsv qeaykkglid yetfkelceq eceweeitit gsdgstrvvl |
| 1921 | vdrktgsqyd iqdaidkglv drkffdqyrs gslsltqfad mislkngvgt sssmgsgvsd |
| 1981 | dvfsssrhes vskistissv rnltirsssf sdtleesspi aaifdtenle kisitegier |
| 2041 | givdsitgqr lleaqactgg iihpttgqkl slqdavsqgv idqdmatrlk paqkafigfe |
| 2101 | gvkgkkkmsa aeavkekwlp yeagqrflef qyltgglvdp evhgristee airkgfidgr |
| 2161 | aaqrlqdtss yakiltcpkt klkisykdai nrsmveditg lrlleaasvs skglpspynm |
| 2221 | ssapgsrsgs rsgsrsgsrs gsrsgsrrgs fdatgnssys ysysfssssi gh |

In some embodiments of the methods of the disclosure, the wild type human DSP gene of the disclosure consists of or comprises the nucleic acid sequence (Genbank Accession number: NM001319034.1, transcript variant 3):

| (SEQ ID NO: 46) | |
|---|---|
| 1 | aagaaaccgg ccaggtgtgg cctaggcgcc cagtgccagc ggggaggaga ctcgctccgc |
| 61 | cgccgaccaa caccaacacc cagctccgac gcagctcctc tgcgcccttg ccgccctccg |
| 121 | agccacagct ttcctcccgc tcctgccccc ggcccgtcgc cgtctccgcg ctcgcagcgg |
| 181 | cctcgggagg gcccaggtag cgagcagcga cctcgcgagc cttccgcact cccgcccggt |
| 241 | tccccggccg tccgcctatc cttggccccc tccgctttct ccgcgccggc ccgcctcgct |
| 301 | tatgcctcgg cgctgagccg ctctcccgat tgcccgccga catgagctgc aacggaggct |
| 361 | cccacccgcg gatcaacact ctgggccgca tgatccgcgc cgagtctggc ccggacctgc |
| 421 | gctacgaggt gaccagcggc ggcgggggca ccagcaggat gtactattct cggcgcggcg |
| 481 | tgatcaccga ccagaactcg gacggctact gtcaaaccgg cacgatgtcc aggcaccaga |
| 541 | accagaacac catccaggag ctgctgcaga actgctccga ctgcttgatg cgagcagagc |
| 601 | tcatcgtgca gcctgaattg aagtatggag atggaataca actgactcgg agtcgagaat |
| 661 | tggatgagtg ttttgcccag gccaatgacc aaatggaaat cctcgacagc ttgatcagag |
| 721 | agatgcggca gatgggccag ccctgtgatg cttaccagaa aaggcttctt cagctccaag |
| 781 | agcaaatgcg agccctttat aaagccatca gtgtccctcg agtccgcagg gccagctcca |
| 841 | agggtggtgg aggctacact tgtcagagtg gctctggctg ggatgagttc accaaacatg |
| 901 | tcaccagtga atgtttgggg tggatgaggc agcaaagggc ggagatggac atggtggcct |
| 961 | ggggtgtgga cctggcctca gtggagcagc acattaacag ccaccggggc atccacaact |
| 1021 | ccatcggcga ctatcgctgg cagctggaca aaatcaaagc cgacctgcgc gagaaatctg |
| 1081 | cgatctacca gttggaggag gagtatgaaa acctgctgaa agcgtccttt gagaggatgg |
| 1141 | atcacctgcg acagctgcag aacatcattc aggccacgtc cagggagatc atgtggatca |
| 1201 | atgactgcga ggaggaggag ctgctgtacg actggagcga caagaacacc aacatcgctc |
| 1261 | agaaacagga ggccttctcc atacgcatga gtcaactgga agttaaagaa aaagagctca |
| 1321 | ataagctgaa acaagaaagt gaccaacttg tcctcaatca gcatccagct tcagacaaaa |
| 1381 | ttgaggccta tatggacact ctgcagacgc agtggagttg gattcttcag atcaccaagt |
| 1441 | gcattgatgt tcatctgaaa gaaaatgctg cctactttca gttttttgaa gaggcgcagt |
| 1501 | ctactgaagc atacctgaag gggctccagg actccatcag gaagaagtac ccctgcgaca |
| 1561 | agaacatgcc cctgcagcac ctgctggaac agatcaagga gctggagaaa gaacgagaga |
| 1621 | aaatccttga atacaagcgt caggtgcaga acttggtaaa caagtctaag aagattgtac |
| 1681 | agctgaagcc tcgtaaccca gactacagaa gcaataaacc cattattctc agagctctct |
| 1741 | gtgactacaa acaagatcag aaaatcgtgc ataaggggga tgagtgtatc ctgaaggaca |
| 1801 | acaacgagcg cagcaagtgg tacgtgacgg gcccgggagg cgttgacatg cttgttccct |
| 1861 | ctgtggggct gatcatccct cctccgaacc cactggccgt ggacctctct tgcaagattg |
| 1921 | agcagtacta cgaagccatc ttggctctgt ggaaccagct ctacatcaac atgaagagcc |
| 1981 | tggtgtcctg gcactactgc atgattgaca tagagaagat cagggccatg acaatcgcca |
| 2041 | agctgaaaac aatgcggcag gaagattaca tgaagacgat agccgacctt gagttacatt |
| 2101 | accaagagtt catcagaaat agccaaggct cagagatgtt tggagatgat gacaagcgga |
| 2161 | aaatacagtc tcagttcacc gatgcccaga agcattacca gaccctggtc attcagctcc |
| 2221 | ctggctatcc ccagcaccag acagtgacca caactgaaat cactcatcat ggaacctgcc |
| 2281 | aagatgtcaa ccataataaa gtaattgaaa ccaacagaga aaatgacaag caagaaacat |
| 2341 | ggatgctgat ggagctgcag aagattcgca ggcagataga gcactgcgag ggcaggatga |
| 2401 | ctctcaaaaa cctccctcta gcagaccagg gatcttctca ccacatcaca gtgaaaatta |
| 2461 | acgagcttaa gagtgtgcag aatgattcac aagcaattgc tgaggttctc aaccagctta |
| 2521 | aagatatgct tgccaacttc agaggttctg aaaagtactg ctatttacag aatgaagtat |
| 2581 | ttggactatt tcagaaactg gaaaatatca atggtgttac agatggctac ttaaatagct |
| 2641 | tatgcacagt aagggcactg ctccaggcta ttctccaaac agaagacatg ttaaaggttt |
| 2701 | atgaagccag gctcactgag gaggaaactg tctgcctgga cctggataaa gtggaagctt |
| 2761 | accgctgtgg actgaagaaa ataaaaaatg acttgaactt gaagaagtcg ttgttggcca |
| 2821 | ctatgaagac agaactacag aaagcccagc agatccactc tcagacttca cagcagtatc |
| 2881 | cactttatga tctggacttg ggcaagttcg gtgaaaaagt cacacagctg acagaccgct |
| 2941 | ggcaaaggat agataaacag atcgacttta ggttatggga cctggagaaa caaatcaagc |
| 3001 | aattgaggaa ttatcgtgat aactatcagg ctttctgcaa gtggctctat gatgctaaac |
| 3061 | gccgccagga ttccttagaa tccatgaaat ttggagattc caacacagtc atgcggtttt |
| 3121 | tgaatgagca gaagaacttg cacagtgaaa tatctggcaa acgagacaaa tcagaggaag |
| 3181 | tacaaaaaat tgctgaactt tgcgccaatt caattaagga ttatgagctc cagctggcct |
| 3241 | catacacctc aggactggaa actctgctga acatacctat caagaggacc atgattcagt |
| 3301 | ccccttctgg ggtgattctg caagaggctg cagatgttca tgctcggtac attgaactac |
| 3361 | ttacaagatc tggagactat tacaggttct taagtgagat gctgaagagt ttggaagatc |
| 3421 | tgaagctgaa aaataccaag atcgaagttt tggaagagga gctcagactg gcccgagatg |
| 3481 | ccaactcgga aaactgtaat aagaacaaat tcctggatca gaacctgcag aaataccagg |
| 3541 | cagagtgttc ccagttcaaa gcgaagcttg cgagcctgga ggagctgaag agacaggctg |
| 3601 | agctggatgg gaagtcggct aagcaaaatc tagacaagtg ctacggccaa ataaaagaac |
| 3661 | tcaatgagaa gatcacccga ctgacttatg agattgaaga tgaaaagaga agaagaaaat |
| 3721 | ctgtggaaga cagatttgac caacagaaga atgactatga ccaactgcag aaagcaaggc |
| 3781 | aatgtgaaaa ggagaacctt ggttggcaga aattagagtc tgagaaagcc atcaaggaga |
| 3841 | aggagtacga gattgaaagg ttgagggttc tactgcagga agaaggcacc cggaagagag |
| 3901 | aatatgaaaa tgagctggca aaggtaagaa accactataa tgaggagatg agtaatttaa |
| 3961 | ggaacaagta tgaaacagag attaacatta cgaagaccac catcaaggag atatccatgc |
| 4021 | aaaaagagga tgattccaaa aatcttagaa accagcttga tagactttca agggaaaatc |
| 4081 | gagatctgaa ggatgaaatt gtcaggctca atgacagcat cttgcaggcc actgagcagc |
| 4141 | gaaggcgagc tgaagaaaac gcccttcagc aaaaggcctg tggctctgag ataatgcaga |
| 4201 | agaagcagca tctggagata gaactgaagc aggtcatgca gcagcgctct gaggacaatg |
| 4261 | cccggcacaa gcagtccctg gaggaggctg ccaagaccat tcaggacaaa aataaggaga |
| 4321 | tcgagagact caaagctgag tttcaggagg aggccaagcg ccgctgggaa tatgaaaatg |
| 4381 | aactgagtaa ggcatctaat aggattcagg aatcaaagaa tcagtgtact caggtggtac |
| 4441 | aggaaagaga gagccttctg gtgaaaatca aagtcctgga gcaagacaag gcaaggctgc |
| 4501 | agaggctgga ggatgagctg aatcgtgcaa aatcaactct agaggcagaa accagggtga |
| 4561 | aacagcgcct ggagtgtgag aaacagcaaa ttcagaatga cctgaatcag tggaagactc |
| 4621 | aatattcccg caaggaggag gctattagga agatagaatc ggaaagagaa aagagtgaga |
| 4681 | gagagaagaa cagtcttagg agtgagatcg aaagactcca agcagagatc aagagaattg |
| 4741 | aagagaggtg caggcgtaag ctggaggatt ctaccaggga gacacagtca cagttagaaa |
| 4801 | cagaacgctc ccgatatcag agggagattg ataaactcag acagcgccca tatgggtccc |
| 4861 | atcgagagac ccagactgag tgtgagtgga ccgttgacac ctccaagctg gtgtttgatg |
| 4921 | ggctgaggaa gaaggtgaca gcaatgcagc tctatgagtg tcagctgatc gacaaaacaa |
| 4981 | ccttggacaa actattgaag gggaagaagt cagtggaaga agttgcttct gaaatccagc |
| 5041 | cattccttcg gggtgcagga tctatcgctg gagcatctgc ttctcctaag gaaaaatact |
| 5101 | ctttggtaga ggccaagaga aagaaattaa tcagcccaga atccacagtc atgcttctgg |
| 5161 | aggcccaggc agctacaggt ggtataattg atccccatcg gaatgagaag ctgactgtcg |
| 5221 | acagtgccat agctcgggac ctcattgact tcgatgaccg tcagcagata tatgcagcag |
| 5281 | aaaaagctat cactggtttt gatgatccat tttcaggcaa gacagtatct gtttcagaag |
| 5341 | ccatcaagaa aaatttgatt gatagagaaa ccggaatgcg cctgctggaa gcccagattg |
| 5401 | cttcaggggg tgtagtagac cctgtgaaca gtgtcttttt gccaaaagat gtcgccttgg |
| 5461 | cccgggggct gattgataga gatttgtatc gatccctgaa tgatccccga gatagtcaga |
| 5521 | aaaactttgt ggatccagtc accaaaaaga aggtcagtta cgtgcagctg aaggaacggt |
| 5581 | gcagaatcga accacatact ggtctgctct tgctttcagt acagaagaga agcatgtcct |
| 5641 | tccaaggaat cagacaacct gtgaccgtca ctgagctagt agattctggt atattgagac |
| 5701 | cgtccactgt caatgaactg gaatctggtc agatttctta tgacgaggtt ggtgagagaa |
| 5761 | ttaaggactt cctccagggt tcaagctgca tagcaggcat atacaatgag accacaaaac |
| 5821 | agaagcttgg catttatgag gccatgaaaa ttggcttagt ccgacctggt actgctctgg |
| 5881 | agttgctgga agcccaagca gctactggct ttatagtgga tcctgttagc aacttgaggt |
| 5941 | taccagtgga ggaagcctac aagagaggtc tggtgggcat tgagttcaaa gagaagctcc |
| 6001 | tgtctgcaga acgagctgtc actgggtata atgatcctga aacaggaaac atcatctctt |
| 6061 | tgttccaagc catgaataag gaactcatcg aaaagggcca cggtattcgc ttattagaag |
| 6121 | cacagatcgc aaccgggggg atcattgacc caaaggagag ccatcgttta ccagttgaca |
| 6181 | tagcatataa gaggggctat ttcaatgagg aactcagtga gattctctca gatccaagtg |
| 6241 | atgataccaa aggatttttt gaccccaaca ctgaagaaaa tcttacctat ctgcaactaa |
| 6301 | aagaaagatg cattaaggat gaggaaacag ggctctgtct tctgcctctg aaagaaaaga |
| 6361 | agaaacaggt gcagacatca caaaagaata ccctcaggaa gcgtagagtg gtcatagttg |
| 6421 | acccagaaac caataaagaa atgtctgttc aggaggccta caagaagggc ctaattgatt |
| 6481 | atgaaacctt caaagaactg tgtgagcagg aatgtgaatg ggaagaaata accatcacgg |
| 6541 | gatcagatgg ctccaccagg gtggtcctgg tagatagaaa gacaggcagt cagtatgata |
| 6601 | ttcaagatgc tattgacaag ggccttgttg acaggaagtt ctttgatcag taccgatccg |
| 6661 | gcagcctcag cctcactcaa tttgctgaca tgatctcctt gaaaaatggt gtcggcacca |
| 6721 | gcagcagcat gggcagtggt gtcagcgatg atgtttttag cagctcccga catgaatcag |
| 6781 | taagtaagat ttccaccata tccagcgtca ggaatttaac cataaggagc agctcttttt |
| 6841 | cagacaccct ggaagaatcg agccccattg cagccatctt tgacacagaa aacctggaga |
| 6901 | aaatctccat tacagaaggt atagagcggg gcatcgttga cagcatcacg ggtcagaggc |
| 6961 | ttctggaggc tcaggcctgc acaggtggca tcatccaccc aaccacgggc cagaagctgt |
| 7021 | cacttcagga cgcagtctcc cagggtgtga ttgaccaaga catggccacc aggctgaagc |
| 7081 | ctgctcagaa agccttcata ggcttcgagg gtgtgaaggg aaagaagaag atgtcagcag |
| 7141 | cagaggcagt gaaagaaaaa tggctcccgt atgaggctgg ccagcgcttc ctggagttcc |
| 7201 | agtacctcac gggaggtctt gttgacccgg aagtgcatgg gaggataagc accgaagaag |
| 7261 | ccatccggaa ggggttcata gatggccgcg ccgcacagag gctgcaagac accagcagct |
| 7321 | atgccaaaat cctgacctgc cccaaaacca aattaaaaat atcctataag gatgccataa |
| 7381 | atcgctccat ggtagaagat atcactgggc tgcgccttct ggaagccgcc tccgtgtcgt |
| 7441 | ccaagggctt acccagccct tacaacatgt cttcggctcc ggggtcccgc tccggctccc |
| 7501 | gctcgggatc tcgctccgga tctcgctccg ggtcccgcag tgggtcccgg agaggaagct |
| 7561 | ttgacgccac agggaattct tcctactctt attcctactc atttagcagt agttctattg |
| 7621 | ggcactagta gtcagttggg agtggttgct ataccttgac ttcatttata tgaatttcca |
| 7681 | ctttattaaa taatagaaaa gaaaatcccg gtgcttgcag tagagtgata ggacattcta |
| 7741 | tgcttacaga aaatatagcc atgattgaaa tcaaatagta aaggctgttc tggcttttta |
| 7801 | tcttcttagc tcatcttaaa taagcagtac acttggatgc agtgcgtctg aagtgctaat |
| 7861 | cagttgtaac aatagcacaa atcgaactta ggatttgttt cttctcttct gtgtttcgat |
| 7921 | ttttgatcaa ttctttaatt ttggaagcct ataatacagt tttctattct tggagataaa |
| 7981 | aattaaatgg atcactgata ttttagtcat tctgcttctc atctaaatat ttccatattc |
| 8041 | tgtattagga gaaaattacc ctcccagcac cagcccccct ctcaaacccc caacccaaaa |
| 8101 | ccaagcattt tggaatgagt ctcctttagt ttcagagtgt ggattgtata acccatatac |
| 8161 | tcttcgatgt acttgtttgg tttggtatta atttgactgt gcatgacagc ggcaatcttt |
| 8221 | tctttggtca aagttttctg tttattttgc ttgtcatatt cgatgtactt taaggtgtct |
| 8281 | ttatgaagtt tgctattctg gcaataaact tttagacttt tgaagtgttt gtgttttaat |
| 8341 | ttaatatgtt tataagcatg tataaacatt tagcatattt ttatcatagg tctaaaaata |
| 8401 | tttgtttact aaatacctgt gaagaaatac cattaaaaaa ctatttggtt ctgaattctt |
| 8461 | actagaaaaa aaa |

In some embodiments of the methods of the disclosure, the wild type human DSP gene of the disclosure consists of or comprises the amino acid sequence (Genbank Accession number: NP_001305963.1, transcript variant 3):

| (SEQ ID NO: 47) | |
|---|---|
| 1 | mscnggshpr intlgrmira esgpdlryev tsggggtsrm yysrrgvitd qnsdgycqtg |
| 61 | tmsrhqnqnt iqellqncsd clmraelivq pelkygdgiq ltrsreldec faqandqmei |
| 121 | ldsliremrq mgqpcdayqk rllqlqeqmr alykaisvpr vrrasskggg gytcqsgsgw |
| 181 | deftkhvtse clgwmrqqra emdmvawgvd lasveqhins hrgihnsigd yrwqldkika |
| 241 | dlreksaiyq leeeyenllk asfermdhlr qlqniiqats reimwindce eeellydwsd |
| 301 | kntniaqkqe afsirmsqle vkekelnklk qesdqlvlnq hpasdkieay mdtlqtqwsw |
| 361 | ilqitkcidv hlkenaayfq ffeeaqstea ylkglqdsir kkypcdknmp lqhlleqike |
| 421 | lekerekile ykrqvqnlvn kskkivqlkp rnpdyrsnkp iilralcdyk qdqkivhkgd |
| 481 | ecilkdnner skwyvtgpgg vdmlvpsvgl iipppnplav dlsckieqyy eailalwnql |
| 541 | yinmkslvsw hycmidieki ramtiaklkt mrqedymkti adlelhyqef irnsqgsemf |
| 601 | gdddkrkiqs qftdaqkhyq tlviqlpgyp qhqtvtttei thhgtcqdvn hnkvietnre |
| 661 | ndkqetwmlm elqkirrqie hcegrmtlkn lpladqgssh hitvkinelk svqndsqaia |
| 721 | evlnqlkdml anfrgsekyc ylqnevfglf qkleningvt dgylnslctv rallqailqt |
| 781 | edmlkvyear lteeetvcld ldkveayrcg lkkikndlnl kksllatmkt elqkaqqihs |
| 841 | qtsqqyplyd ldlgkfgekv tqltdrwqri dkqidfrlwd lekqikqlrn yrdnyqafck |
| 901 | wlydakrrqd slesmkfgds ntvmrflneq knlhseisgk rdkseevqki aelcansikd |
| 961 | yelqlasyts gletllnipi krtmiqspsg vilqeaadvh aryielltrs gdyyrflsem |
| 1021 | lksledlklk ntkievleee lrlardanse ncnknkfldq nlqkyqaecs qfkaklasle |
| 1081 | elkrqaeldg ksakqnldkc ygqikelnek itrltyeied ekrrrksved rfdqqkndyd |
| 1141 | qlqkarqcek enlgwqkles ekaikekeye ierlrvllqe egtrkreyen elakvrnhyn |
| 1201 | eemsnlrnky eteinitktt ikeismqked dsknlrnqld rlsrenrdlk deivrlndsi |
| 1261 | lqateqrrra eenalqqkac gseimqkkqh leielkqvmq qrsednarhk qsleeaakti |
| 1321 | qdknkeierl kaefqeeakr rweyenelsk asnriqeskn qctqvvqere sllvkikvle |
| 1381 | qdkarlqrle delnrakstl eaetrvkqrl ecekqqiqnd lnqwktqysr keeairkies |
| 1441 | erekserekn slrseierlq aeikrieerc rrkledstre tqsqleters ryqreidklr |
| 1501 | qrpygshret qtecewtvdt sklvfdglrk kvtamqlyec qlidkttldk llkgkksvee |
| 1561 | vaseiqpflr gagsiagasa spkekyslve akrkklispe stvmlleaqa atggiidphr |
| 1621 | nekltvdsai ardlidfddr qqiyaaekai tgfddpfsgk tvsyseaikk nlidretgmr |
| 1681 | lleaqiasgg vvdpvnsvfl pkdvalargl idrdlyrsln dprdsqknfv dpvtkkkvsy |
| 1741 | vqlkercrie phtgllllsv qkrsmsfqgi rqpvtvtelv dsgilrpstv nelesgqisy |
| 1801 | devgerikdf lqgssciagi ynettkqklg iyeamkiglv rpgtalelle aqaatgfivd |
| 1861 | pvsnlrlpve eaykrglvgi efkekllsae ravtgyndpe tgniislfqa mnkeliekgh |
| 1921 | girlleaqia tggiidpkes hrlpvdiayk rgyfneelse ilsdpsddtk gffdpnteen |
| 1981 | ltylqlkerc ikdeetglcl lplkekkkqv qtsqkntlrk rrvvivdpet nkemsvqeay |
| 2041 | kkglidyetf kelceqecew eeititgsdg strvvlvdrk tgsqydiqda idkglvdrkf |
| 2101 | fdqyrsgsls ltqfadmisl kngvgtsssm gsgvsddvfs ssrhesvski stissvrnlt |
| 2161 | irsssfsdtl eesspiaaif dtenlekisi tegiergivd sitgqrllea qactggiihp |
| 2221 | ttgqklslqd aysqgvidqd matrlkpaqk afigfegvkg kkkmsaaeav kekwlpyeag |
| 2281 | qrflefqylt gglvdpevhg risteeairk gfidgraaqr lqdtssyaki ltcpktklki |
| 2341 | sykdainrsm veditglrll eaasysskgl pspynmssap gsrsgsrsgs rsgsrsgsrs |
| 2401 | gsrrgsfdat gnssysysys fssssigh |

In some embodiments of the methods of the disclosure, the wild type human AZGP1 gene of the disclosure consists of or comprises the nucleic acid sequence (Genbank Accession number: NM_001185.3):

| (SEQ ID NO: 21) | |
|---|---|
| 1 | ccattggcct gtagattcac ctcccctggg cagggcccca ggacccagga taatatctgt |
| 61 | gcctcctgcc cagaaccctc caagcagaca caatggtaag aatggtgcct gtcctgctgt |
| 121 | ctctgctgct gcttctgggt cctgctgtcc cccaggagaa ccaagatggt cgttactctc |
| 181 | tgacctatat ctacactggg ctgtccaagc atgttgaaga cgtccccgcg tttcaggccc |
| 241 | ttggctcact caatgacctc cagttcttta gatacaacag taaagacagg aagtctcagc |
| 301 | ccatgggact ctggagacag gtggaaggaa tggaggattg gaagcaggac agccaacttc |
| 361 | agaaggccag ggaggacatc tttatggaga ccctgaaaga catcgtggag tattacaacg |
| 421 | acagtaacgg gtctcacgta ttgcagggaa ggtttggttg tgagatcgag aataacagaa |
| 481 | gcagcggagc attctggaaa tattactatg atggaaagga ctacattgaa ttcaacaaag |
| 541 | aaatcccagc ctgggtcccc ttcgacccag cagcccagat aaccaagcag aagtgggagg |
| 601 | cagaaccagt ctacgtgcag cgggccaagg cttacctgga ggaggagtgc cctgcgactc |
| 661 | tgcggaaata cctgaaatac agcaaaaata tcctggaccg gcaagatcct ccctctgtgg |
| 721 | tggtcaccag ccaccaggcc ccaggagaaa agaagaaact gaagtgcctg gcctacgact |
| 781 | tctacccagg gaaaattgat gtgcactgga ctcgggccgg cgaggtgcag gagcctgagt |
| 841 | tacggggaga tgttcttcac aatggaaatg gcacttacca gtcctgggtg gtggtggcag |
| 901 | tgcccccgca ggacacagcc ccctactcct gccacgtgca gcacagcagc ctggcccagc |
| 961 | ccctcgtggt gccctgggag gccagctagg aagcaagggt tggaggcaat gtgggatctc |
| 1021 | agacccagta gctgcccttc ctgcctgatg tgggagctga accacagaaa tcacagtcaa |
| 1081 | tggatccaca aggcctgagg agcagtgtgg ggggacagac aggaggtgga tttggagacc |
| 1141 | gaagactggg atgcctgtct tgagtagact tggacccaaa aaatcatctc accttgagcc |
| 1201 | cacccccacc ccattgtcta atctgtagaa gctaataaat aatcatccct ccttgcctag |
| 1261 | cataaaaaaa aaaaaaaa |

In some embodiments of the methods of the disclosure, the wild type human AZGP1 gene of the disclosure consists of or comprises the nucleic acid sequence (Genbank Accession number: NP_001176.1):

| (SEQ ID NO: 22) | |
|---|---|
| 1 | mvrmvpvlls lllllgpavp qenqdgrysl tyiytglskh vedvpafqal gslndlqffr |
| 61 | ynskdrksqp mglwrqvegm edwkqdsqlq karedifmet lkdiveyynd sngshvlqgr |
| 121 | fgceiennrs sgafwkyyyd gkdyiefnke ipawvpfdpa aqitkqkwea epvyvqraka |
| 181 | yleeecpatl rkylkyskni ldrqdppsvv vtshqapgek kklkclaydf ypgkidvhwt |
| 241 | ragevqepel rgdvlhngng tyqswvvvav ppqdtapysc hvqhsslaqp lvvpweas |

In some embodiments of the methods of the disclosure, the wild type human OBFC1 gene of the disclosure consists of or comprises the nucleic acid sequence (Genbank Accession number: NM_024928):

| (SEQ ID NO: 23) | |
|---|---|
| 1 | aaatgcgctg gcggggagac cggggttggt ccctggcggg gcagggggcg ggctcaggcc |
| 61 | ggaactccag agacgacctc agccaactgc tcctgcgccg ggcggggtcg tcgccgccag |
| 121 | cggctccgag cgccggaagg gccaggtctc agggctcctg gagctgcagg cggcgggagg |
| 181 | ggctacaaat gcttgactca gtgatgcaga acctttcaga gttagctgga agccacagcc |
| 241 | ctgcctcttg atgcagcctg gatccagccg gtgtgaagag gagacccctt ccctcttgtg |
| 301 | gggtttggat cctgtgtttc tagcctttgc aaaactctac atcagggata tcctggacat |
| 361 | gaaggagtcc cgccaggtgc caggtgtatt tttgtacaat ggacatccaa taaaacaggt |
| 421 | agatgtcttg ggaactgtca ttggagtgag agaaagagat gctttctaca gttatggagt |
| 481 | ggatgacagc actggagtta taaactgcat ctgctggaaa aagttgaata ctgagtctgt |
| 541 | atcagctgct ccaagtgcag caagagagct cagcttaacc tcacaactta agaagctaca |
| 601 | agagaccatt gagcagaaaa caaagataga gatcggggac acgatccgag tcagaggcag |
| 661 | tatccgcaca tacagagaag agcgagagat tcatgccacc acttactata aagtggacga |
| 721 | cccagtgtgg aacattcaaa ttgcaaggat gcttgagctg cccactatct acaggaaagt |
| 781 | ttatgaccag ccttttcaca gctcagccct agagaaagaa gaggcactaa gcaatccagg |
| 841 | cgccctggac ctccccagtc tcacgagttt gctgagtgaa aaagccaaag aattcctcat |
| 901 | ggagaacaga gtgcagagct tttaccagca ggagctggaa atggtggagt ctttgctgtc |
| 961 | ccttgccaat cagcctgtga ttcacagtgc ctcctccgac caagtgaatt ttaagaagga |
| 1021 | caccacttcc aaggcaattc atagtatatt taagaatgct atacaactgc tgcaggaaaa |
| 1081 | aggacttgtt ttccagaaag atgatggttt tgataaccta tactatgtaa ccagagaaga |
| 1141 | caaagacctg cacagaaaga tccaccggat cattcagcag gactgccaga aaccaaatca |
| 1201 | catggagaag ggctgtcact tcctgcacat cttggcctgt gctcgcctga gcatccgccc |
| 1261 | gggcctgagc gaggctgtgc tgcagcaagt tctggagctc ctggaggacc agagtgacat |
| 1321 | tgtcagcaca atggagcact actacacagc gttctgagca gagacacgca gaccagctga |
| 1381 | ggaggacaaa gataaggtgg cattcacccc caggctctga ctttcagcat catgcagggg |
| 1441 | cttatctgtc tggaggcagt tacctcataa taaactataa aatatagtca tcttgggaat |
| 1501 | gggatttggc ataaatgttg ttggctccct tctgtccact atgtccttgg tgtacaatga |
| 1561 | ctttgatctc agccatgaca caacaagaaa accctccctg ttgagctcct ggctggactg |
| 1621 | tgcgttgttc gcagagcaga atggggagga aacagtgttg gcagcttaac tgatgtgtgt |
| 1681 | ggttggagtc tcttccatgg caaagggaca ccacagggta gtgaacattc aggaactgag |
| 1741 | gggcatatgg cctgatcaca cagttctaag cttttcaaaa cttcaggtta tcagagacct |
| 1801 | tcctgtgggc ctctcttgct ggctaagaac cggtttaggg gagtagttct ccctggatga |
| 1861 | gtgcttacag tttctgtggc tcagttacca gcagtggggt tgagacctgg gtcgatgctc |
| 1921 | tttacaggcc tgcccagaga tgggaataaa cagggatcca cagcgtgact atgtgtttgt |
| 1981 | cattttcctt ttatttcctt gggaatcgaa aggtgtccca gtacatttcc ctgcacttac |
| 2041 | agaggtgcat gactaaatac attgtccctc gatgcccctg aagatcacgg aggcagtcag |
| 2101 | ccaattgcct ggcaggtggt agatgttatt ttcagggttg ccgctgagtg tgcaggatgt |
| 2161 | gctgacacca tccagacaaa gactcggtat gtgcccagac aggtgatgga gtcatgcttt |
| 2221 | tgctcagaat gacaaggtaa aggaaaaaca tctgaggtat gttgtaggcc tgttctgaca |
| 2281 | gcaaaatgac aaatccagcc agcaaaaata aagtgtggag aaagatttgg agttaattac |
| 2341 | agtcatttca cagaaggcac tgccttcgtc tgctgcattt gctcttgatg tgataagctc |
| 2401 | ttcgtggctc agctggagat cctttaggcc tggagagttg ctcctctctc cgtggaaaca |
| 2461 | ggacagtctt tatacgcaga agtccgctgc agctcgatac gtcaggctga gagctagaac |
| 2521 | cagtagattg cctcctgtca tagacttttg taatgatgca aacctttgct gatttctaac |
| 2581 | agtgattatg tagtggctgc cctgcatctt ctctgtgtac agaagggtcc ctagcataga |
| 2641 | gtctgcctgg aatgatgtcc tgggcagttc ttccttgagg tcagcagctg ttccacgttg |
| 2701 | aatgcatctg attagtgggg ctgcccagga aggagttcag aatcagaagg taaaaagggc |
| 2761 | atacccttgc ctatagcaac tctgctctta ggggtttatc tcaaggagat ggctacacaa |
| 2821 | gtgtgaaagg atggttgcac aaggtgttca ttgctgtata atctagaatt ctatattggg |
| 2881 | gaaaatacct atagggaaaa agttaattac ggttcttggg cacaatgaaa tactatgcag |
| 2941 | ctatgaaaaa aatgatgaaa gcagacagac agtgttgcca tggcacactg tccctagtag |
| 3001 | atttagtggg aagtagatag agttatagat ctgtttctat agtataacac cattatctac |
| 3061 | agctccctgt gtgtatgtat atatccgtag agagagtgta tatttctgca tggaggtctt |
| 3121 | tataaatgta gcacatgtac atatatatat atatacacac acacagtcga ccactccctt |
| 3181 | ctcctggaag tactttccgc gtttggcttt caggacacca agctctctgg ttgctccttc |
| 3241 | tcaggttcct ttgttcagtg ctctgcctcc ctgaggactc agtcccagac ctcttttcta |
| 3301 | tctggcttgc tcactggggt gtctccagca gccacatgga ttataccatc tacatgctgt |
| 3361 | ctaacacctc agtttaaacc cagaatgggc ctcttccctg aactgcagac ccctatattc |
| 3421 | agtttgctac tgacatctcc acttaggtct ctaatggaca tctcagattt cacaggccca |
| 3481 | aagccaggct cccaattact cctgacccca ggcttgctcc tgatagtgac atgaggcagc |
| 3541 | caaatgccta ggcagagagg ggagggtccc aaatgaaacc ccacgttcaa gcaaagatca |
| 3601 | gcctgaaggc taaaagacca gattgctggt cctggatgaa acccaccacg cagagtggga |
| 3661 | acttctgttc ctgtttgccc accctttccc aattgttctt tctgaataac gccttaacca |
| 3721 | atcgaatgtt gccttttcca gtaataccta cagcctgccc ctccccccat tctgagccca |
| 3781 | taaaaagacc cagactcccc catattaagg ggactttcct gcctttgggt agggggacca |
| 3841 | cccccacgtc tcctctctgt tgaaaactgt ttcatcactc aataaaactc ccagctttgc |
| 3901 | tcactcttcc actgtcagca cattctcatt cttctttggt gctgggcaag aactcaacca |
| 3961 | gtgtggaagc catacttggc ccaggcgggt gaagtgggcg ggccgtctcc tgcagcaggt |
| 4021 | agcatggtca agcgaggccc aggtgggccg tcaccagcca gaggtccctg gcttgcaaag |
| 4081 | tgaccgagaa aaaaatcctg tgccactcct ttggaaaatg tccctgattc aggaagaggt |
| 4141 | agctccatcc agttgctcaa accaaatcca ttggcttctt tctttctatc atacctcaca |
| 4201 | tccaatctgt ctgcaagtct tttggctcta ccttcagaat atctccagaa tcttaactgc |
| 4261 | ttcaccctcc tccccggcct cctcagtcct ctctgcttcc gccctggccc ctcttgggct |
| 4321 | gttcacagca cagcagctgt tgccaccctg ttaatgctcc cactctccta cagccttcgg |
| 4381 | tcttgcccca ggtaggagcc tgaggctgca cagaggtcag cacggccccg cttaccctgc |
| 4441 | cctcccagcc cagccgcacg ggccttgcac acatgcctcg gcatattcct gccttagggc |
| 4501 | tggtgctcct gctatttcct cttcccaggt aaccatgtga agtgcctccc tctgccctct |
| 4561 | ttccagcctt tacttgagtg tcaccttctc agtgaggcct gccctcattc ctctttcgct |
| 4621 | gtttgcaacc catctcctgt cccccttccc agaactccct ttcctacttc gtttttcttc |
| 4681 | acagtacttg atactgccta acacactcca tggtttctta cttgccctgt ttattatttt |
| 4741 | cccccaatag acagaatgtt ccatgatggc agaattctct gttttgtttc cttccatgtc |
| 4801 | cccagcacct agaacagtgc ctgacgcatc tcctaagcaa tacgaccaat aagtatgtgt |
| 4861 | ctggctgcct tccggctgcc agtgtctgcc tctttcctag gggcagtggt tgcgggggtg |
| 4921 | ctttctcaca tgtcttagta ggctgtgcag gctggaagtg ctcagaagtc acacccccag |
| 4981 | ggagcagcct cagccaacag caccttggct gtaaatgccc cagctccctc gccctcaggt |
| 5041 | aagcattgct gaggcacacg ttccatactc ttttccacag ttcctccgtg ggactgagca |
| 5101 | ccacccagcc acccacagga gcagctaacc tgataaccac cagcctcacc ctccctgcct |
| 5161 | tacttccccg ctccccttta ccacatgctg acctcccaga tgcatttctt gctttccggt |
| 5221 | ctctgtctca ggattggctc ctggatgaac acaaactaac actatgttca caaatatatt |
| 5281 | tgggaaatgc tggatgaata attatacaca tcagacagat tactagaaat tctcaccaaa |
| 5341 | gggatgcaca tgttacctct gcatggtgag atctcaggtg ctttttaccc cacatagcta |
| 5401 | tcctttggca tttttataat tagcaagtgc tcactcttcc actgtcagta cattctcatt |
| 5461 | cttcttgggc gctggacaag aattcaaccg gtgtgtaagc cagactcggc ccgggcagtc |
| 5521 | tcaaactcct gactccttat ataatttcta caaaaattat aaagctattt cccactcccc |
| 5581 | accccacatt catgtaacct gaagcatgag taaaccaaga atgaggtagg cctctgtctt |
| 5641 | ctaagcaaca tcagaactct aagaacatga gggactctta gaaaactctc tggagctaac |
| 5701 | cacagctggg tcactgctca tgtactgaag accagccaga gggttcccct gaaaaggagg |
| 5761 | gaaactgagc aaacattctc cagttctctt agtgtgcaca tgtttcagga ggtgtgaacc |
| 5821 | ccacatgtag cttgtgtagg caagaagaca aatagtgcta ctgtctggtc aaggatttgt |
| 5881 | ttgaagagcc atgattatgc ccatatggta agccaccagt gctccccatc cctgtaagac |
| 5941 | acttctttct cattattttc tcctctgatg gtgtgccagg atgctggcca agagaagcca |
| 6001 | agtggaaaga aggctgttca gtgacaagga acctaagact tagtgccaag gactgaaacc |
| 6061 | aagtaaactt gtaattttcc atgatggaaa catctacact ttctcattag tggcctctac |
| 6121 | agcagttgcc ccaaagaagc gtctcattgt ttttttacta catttatgtg aagcatacag |
| 6181 | gcaaactcag aaagactgtg ataaggctcg ccagagatgc ctgcacaggt gctgggggaa |
| 6241 | aagcaggacc atcctgaagg gagatggtgt ctgtggacaa agaactctgc agtggttctt |
| 6301 | atttgcatga tttctgctgg tggaggctgt aaatgtgagc tcaaactccc acataagtga |
| 6361 | gttttcattg taatccagaa tgtttttaaa tcaccctact tctattgaac ttgcactatc |
| 6421 | atctgttaac ctctactgta tttattaaat aaacctgaat aggtaaatca cagtacagca |
| 6481 | aaa |

In some embodiments of the methods of the disclosure, the wild type human OBFC1 gene of the disclosure consists of or comprises the amino acid sequence (Genbank Accession number: NP_079204.2):

| (SEQ ID NO: 24) | |
|---|---|
| 1 | mqpgssrcee etpsllwgld pvflafakly irdildmkes rqvpgvflyn ghpikqvdvl |
| 61 | gtvigvrerd afysygvdds tgvincicwk klntesvsaa psaarelslt sqlkklqeti |
| 121 | eqktkieigd tirvrgsirt yreereihat tyykvddpvw niqiarmlel ptiyrkvydq |
| 181 | pfhssaleke ealsnpgald lpsltsllse kakeflmenr vqsfyqqele mvesllslan |
| 241 | qpvihsassd qvnfkkdtts kaihsifkna iqllqekglv fqkddgfdnl yyvtredkdl |
| 301 | hrkihriiqq dcqkpnhmek gchflhilac arlsirpgls eavlqqvlel ledqsdivst |
| 361 | mehyytaf |

In some embodiments of the methods of the disclosure, the wild type human ATP11A gene of the disclosure consists of or comprises the nucleic acid sequence (Genbank Accession number: NM_015205.2, transcript variant 1):

| (SEQ ID NO: 25) | |
|---|---|
| 1 | gcggccgcac tagtaccccg gagcccatgg gcgcgccgag ccgggcgcgg gggcgctgaa |
| 61 | cggcggagcg ggagcggccg gaggagccat ggactgcagc ctcgtgcgga cgctcgtgca |
| 121 | cagatactgt gcaggagaag agaattgggt ggacagcagg accatctacg tgggacacag |
| 181 | ggagccacct ccgggcgcag aggcctacat cccacagaga tacccagaca acaggatcgt |
| 241 | ctcgtccaag tacacatttt ggaactttat acccaagaat ttatttgaac aattcagaag |
| 301 | agtagccaac ttttatttcc ttatcatatt tctggtgcag ttgattattg atacacccac |
| 361 | aagtccagtg acaagcggac ttccactctt ctttgtcatt actgtgacgg ctatcaaaca |
| 421 | gggttatgaa gactggcttc gacataaagc agacaatgcc atgaaccagt gtcctgttca |
| 481 | tttcattcag cacggcaagc tcgttcggaa acaaagtcga aagctgcgag ttggggacat |
| 541 | tgtcatggtt aaggaggacg agacctttcc ctgcgacttg atcttccttt ccagcaaccg |
| 601 | gggagatggg acgtgccacg tcaccaccgc cagcttggat ggagaatcca gccataaaac |
| 661 | gcattacgcg gtccaggaca ccaaaggctt ccacacagag gaggatatcg gcggacttca |
| 721 | cgccaccatc gagtgtgagc agccccagcc cgacctctac aagttcgtgg gtcgcatcaa |
| 781 | cgtttacagt gacctgaatg accccgtggt gaggccctta ggatcggaaa acctgctgct |
| 841 | tagaggagct acactgaaga acactgagaa aatctttggt gtggctattt acacgggaat |
| 901 | ggaaaccaag atggcattaa attatcaatc aaaatctcag aagcgatctg ccgtggaaaa |
| 961 | atcgatgaat gcgttcctca ttgtgtatct ctgcattctg atcagcaaag ccctgataaa |
| 1021 | cactgtgctg aaatacatgt ggcagagtga gccctttcgg gatgagccgt ggtataatca |
| 1081 | gaaaacggag tcggaaaggc agaggaatct gttcctcaag gcattcacgg acttcctggc |
| 1141 | cttcatggtc ctctttaact acatcatccc tgtgtccatg tacgtcacgg tcgagatgca |
| 1201 | gaagttcctc ggctcttact tcatcacctg ggacgaagac atgtttgacg aggagactgg |
| 1261 | cgaggggcct ctggtgaaca cgtcggacct caatgaagag ctgggacagg tggagtacat |
| 1321 | cttcacagac aagaccggca ccctcacgga aaacaacatg gagttcaagg agtgctgcat |
| 1381 | cgaaggccat gtctacgtgc cccacgtcat ctgcaacggg caggtcctcc cagagtcgtc |
| 1441 | aggaatcgac atgattgact cgtcccccag cgtcaacggg agggagcgcg aggagctgtt |
| 1501 | tttccgggcc ctctgtctct gccacaccgt ccaggtgaaa gacgatgaca gcgtagacgg |
| 1561 | ccccaggaaa tcgccggacg gggggaaatc ctgtgtgtac atctcatcct cgcccgacga |
| 1621 | ggtggcgctg gtcgaaggtg tccagagact tggctttacc tacctaaggc tgaaggacaa |
| 1681 | ttacatggag atattaaaca gggagaacca catcgaaagg tttgaattgc tggaaatttt |
| 1741 | gagttttgac tcagtcagaa ggagaatgag tgtaattgta aaatctgcta caggagaaat |
| 1801 | ttatctgttt tgcaaaggag cagattcttc gatattcccc cgagtgatag aaggcaaagt |
| 1861 | tgaccagatc cgagccagag tggagcgtaa cgcagtggag gggctccgaa ctttgtgtgt |
| 1921 | tgcttataaa aggctgatcc aagaagaata tgaaggcatt tgtaagctgc tgcaggctgc |
| 1981 | caaagtggcc cttcaagatc gagagaaaaa gttagcagaa gcctatgagc aaatagagaa |
| 2041 | agatcttact ctgcttggtg ctacagctgt tgaggaccgg ctgcaggaga aagctgcaga |
| 2101 | caccatcgag gccctgcaga aggccgggat caaagtctgg gttctcacgg gagacaagat |
| 2161 | ggagacggcc gcggccacgt gctacgcctg caagctcttc cgcaggaaca cgcagctgct |
| 2221 | ggagctgacc accaagagga tcgaggagca gagcctgcac gacgtcctgt tcgagctgag |
| 2281 | caagacggtc ctgcgccaca gcgggagcct gaccagagac aacctgtccg gactttcagc |
| 2341 | agatatgcag gactacggtt taattatcga cggagctgca ctgtctctga taatgaagcc |
| 2401 | tcgagaagac gggagttccg gcaactacag ggagctcttc ctggaaatct gccggagctg |
| 2461 | cagcgcggtg ctctgctgcc gcatggcgcc cttgcagaag gctcagattg ttaaattaat |
| 2521 | caaattttca aaagagcacc caatcacgtt agcaattggc gatggtgcaa atgatgtcag |
| 2581 | catgattctg gaagcgcacg tgggcatagg tgtcatcggc aaggaaggcc gccaggctgc |
| 2641 | caggaacagc gactatgcaa tcccaaagtt taagcatttg aagaagatgc tgcttgttca |
| 2701 | cgggcatttt tattacatta ggatctctga gctcgtgcag tacttcttct ataagaacgt |
| 2761 | ctgcttcatc ttccctcagt ttttatacca gttcttctgt gggttttcac aacagacttt |
| 2821 | gtacgacacc gcgtatctga ccctctacaa catcagcttc acctccctcc ccatcctcct |
| 2881 | gtacagcctc atggagcagc atgttggcat tgacgtgctc aagagagacc cgaccctgta |
| 2941 | cagggacgtc gccaagaatg ccctgctgcg ctggcgcgtg ttcatctact ggacgctcct |
| 3001 | gggactgttt gacgcactgg tgttcttctt tggtgcttat ttcgtgtttg aaaatacaac |
| 3061 | tgtgacaagc aacgggcaga tatttggaaa ctggacgttt ggaacgctgg tattcaccgt |
| 3121 | gatggtgttc acagttacac taaagcttgc attggacaca cactactgga cttggatcaa |
| 3181 | ccattttgtc atctgggggt cgctgctgtt ctacgttgtc ttttcgcttc tctggggagg |
| 3241 | agtgatctgg ccgttcctca actaccagag gatgtactac gtgttcatcc agatgctgtc |
| 3301 | cagcgggccc gcctggctgg ccatcgtgct gctggtgacc atcagcctcc ttcccgacgt |
| 3361 | cctcaagaaa gtcctgtgcc ggcagctgtg gccaacagca acagagagag tccagactaa |
| 3421 | gagccagtgc ctttctgtcg agcagtcaac catctttatg ctttctcaga cttccagcag |
| 3481 | cctgagtttc tgatggaaca agagcccagg ctaccagagc acctgtccct cggccgcctg |
| 3541 | gtacagctcc cactctcagc aggtgacact cgcggcctgg aaggagaagg tgtccacgga |
| 3601 | gcccccaccc atcctcggcg gttcccatca ccactgcagt tccatcccaa gtcacagctg |
| 3661 | ccctaggtcc cgtgtgggaa tgctcgtgtg atggatggtc ctaagcctgt ggagactgtg |
| 3721 | cacgtgcctc ttcctggccc ccagcaggca aggagggggg tcacaggcct tgccctcgag |
| 3781 | catggcaccc tggccgcctg gacccagcac tgtggttgtt gagccacacc agtggcctct |
| 3841 | gggcattcgg ctcaacgcag gagggacatt ctgctggccc accctgcgcg ctgtcatgca |
| 3901 | gaggccattc ccccaggcct gtgtcttcac ccacctgcca tcattggcct ttgctgtcac |
| 3961 | tgggagagaa gagccgtcca gggacccatg gtggcccaca tgtggatgcc acatgctgct |
| 4021 | gtttcctgct tgcccggcca ccacccatgc cctccatagg gtgaggtgga gccatggtgg |
| 4081 | tgcgtccttt actcaacaac cctccaatcc ggatgctgtg ggaagggccg ggtcactcgg |
| 4141 | ataccatcat ccctgcggat gcaccgccgt accctgctca tctgggagtg gtttccctgc |
| 4201 | ggttacgtcc aagcccgcct gccctgtgtg ttggggctgg ctgagtttcg gtctccccat |
| 4261 | caccggccgc ctcgtggaga aggcagtgcc acgtgggagg acaaggccac gccggcagct |
| 4321 | tccagccctg ccgcagaagt gccaggatgt ccatcagcca ctcgccaggg cacggagccg |
| 4381 | tcagtccact gttacgggag aatgttgatt tcgcgggtgc gagggccggg agacagatac |
| 4441 | ttggctgtga tgagcagaca tcctctgtcc ccgtggaggg gtcaacacca aggtggtgtt |
| 4501 | cgtgcaccag aacctgtctc gggctgacgg gggtggcaca caggacacgg gtggatccca |
| 4561 | acaggcagca ccgcacctct gcccgcctcc cgcactgcag ctccgcccgc cgggctctgc |
| 4621 | gtccccacgt cccctcgtcc catccccacg tcccctcatc ccgtcacctc gtccccacat |
| 4681 | ccccttgccc cgtcacctcg tcctcatgtc cccttgtcct gtcacctcgt ccccacgtcc |
| 4741 | cctcgtctcc tcatccccac gtcctctcgt ccccttgtcc cgtccccaca taccctcgtc |
| 4801 | cccatgtccc cacgcagggc tctccttcgt cttaggatct gtccagcgct gctctgggtg |
| 4861 | ggttagcaac cccagggctg ctgtgatagg aagtccctgt tgttctccgt actggcattt |
| 4921 | ctatttctag aaataatatt tgacatagcc ttaatggtcc ttaaagaaga catttcagtg |
| 4981 | tgagattcag acttcagacg ctgaaactgc tgcctttcag gaaagcacca ccaacgctgg |
| 5041 | aggaggagcc ggccctcacg cccgccccgc gccacgctgt ggaacggggc tccggcaagt |
| 5101 | gaaacccaga gggtgtttcc gaggtgctcg acagtaggta tttttggaag ctcagatttc |
| 5161 | accatttgat tgtataatct tttacctata aaatatttat ttgaagtaga gggtaaatca |
| 5221 | gcggtaagaa cagtgaacac agtggttggg ataaaataag gtgacaaaca tcacaccaaa |
| 5281 | gatgagggta gcgagcaact ggcttgagca gacagaacgg ggaagactcc actctgtccc |
| 5341 | gaggggccag ccgcaggcgt ccccagggcc accctgccct gaggtccttg tgtggccgcc |
| 5401 | ctggcttggc agccctgccc acgctgcccc cgcaaacaat ggtgtgtgcg tttttacagc |
| 5461 | cctttttagg aacccaatat gggcataaat gtaacacctg tagcgggggc agattctctg |
| 5521 | tatgttcagt taacaaatta tttgtaatgt atttttttag aaatcttaaa attgcctttg |
| 5581 | cactgaagta ttttcatagc tgtttatatc tcttttattc atttatttaa catactgtct |
| 5641 | aattttaaaa ataggttttt aaagctttca tttttaagtt tatgaaattt tggccacttt |
| 5701 | acatttagat tctggtgaga gttttgactg aatgttccaa tctctgatga atgcgaattt |
| 5761 | tcagatttga ttttattctc tacacacacc tcttcttttc ttggtatttc tggtggcagt |
| 5821 | gattagttga acagcacatt taaggcacga taatttgcta cactttttct ttacaatttg |
| 5881 | ttgcaatttc atctgctttc tatgtttcat tgttaattgc catccttcag ccttaaaaat |
| 5941 | agaagattct cacgtgaagg tttagtaagt tgggtcccag ctctgcctgt gtggagatag |
| 6001 | tcaccatgta cctctgacaa caagttttag tgtgaaagtc actaaacttt tacacactcc |
| 6061 | caaacgtctt tttaaaaatt gcttgggaaa ttattaaatg aatgtgcctg atgatttgaa |
| 6121 | atagacaagg ggcacgagat aaaaaagaaa aggatgagaa gatcctcagt gaatgacgtt |
| 6181 | gcagggtctt catgcaattt tccacctcgc agtagttagt atttacttgc cttaaactaa |
| 6241 | ctttgaagca agtaatgtca actttgagca ctttgttgag ttttgaaaaa tcttatttgt |
| 6301 | tgctgcacag gttaataaat tatcaatttg taattcagca tgttggtcag agacacggtc |
| 6361 | actgattcac acccagtccc tgccacagac cgtctcagac acgcacagtg ggcctgctgc |
| 6421 | atgattcaca cccagtccct gccacagacc gtctcagaca cgcacagtgg gcctgctgca |
| 6481 | tgattcacac ccagtccctg ccacagaccg tctcagacac gcacagtggg cctgctgcat |
| 6541 | gcgtgttacc tggcttttgg ctccacgctc actcatagcc atgtccacat gggggcttgc |
| 6601 | acacaggatc actcacatat gtacatgtac ccaccacaaa cgtgcaagct cctgcacaca |
| 6661 | tgcatgcaca caaacgtgta cacaagtgtg agctcctaca cgcatacaca cacacacgtg |
| 6721 | tacatgcacc aaagcatgtg tgacctacag acatgcagaa catgcacgtg tacacatacc |
| 6781 | acagacacgc gtgtgcatgc tcctacacaa tacatatgca catatcatga acagcgtaag |
| 6841 | ttcctacaca cggacgtgtg atacacacat gcatgtacag gtaagcacac atgtacaagc |
| 6901 | tcctacaggc ttgctctcac acacgtgtat gcacagcaga gagacgtatg agcttctact |
| 6961 | gcacacatgc acacacacac gcacacgtac attcactaca aacgtgcagc ctcctgcaca |
| 7021 | cgtgcacatt catgtgtaca ccacaaatga gttcccagac gtgtaaacac acgtgcacac |
| 7081 | atcgtacaca tgtgagctcc cacacgtaca cacagatgca catggacaca ccccaaacac |
| 7141 | gcacaggctc ctacacacat gcacacacgt gtacaccaca aacgagctcc cagacatgta |
| 7201 | aacacacgtc tcccacacgt gagctcccac acgtacacat gcacatgtac gcaccacaaa |
| 7261 | cacatgcgca ggctcctgca ggcgtgaata cacacatgca cacacatata cacacatgtg |
| 7321 | ccacaaacaa gtgcacactg tcctggtgtc ctgcactgca tcctgcctcc ttgctgaggg |
| 7381 | gcccctgtga gaggcctctg gatgggcatg ggaagatggg ctccctggcc cccagcccat |
| 7441 | gcctccctgg gatgaagagt ccccctcctg gcagaatgtc tgggctttgc agagcaggcc |
| 7501 | ccgggggtga agtcgcagct tcacttacac cagctgctct gtgagcaagg cttggtgccc |
| 7561 | tggacaaggc ccttcccctt tagggaggtc cagcctcgca agctgaaacc tcccctcggc |
| 7621 | tcagccctat accaggcggc cacagcagga ctggccacac ccacgccgca cctcatccgt |
| 7681 | gcacgcgtcg gagcacggcc agccttccgc cacgagccag ctgggaaggg ccgcggccgc |
| 7741 | ctaaagcccc agtcaaccca gcctgtgtct gagcagacag ggcgaacaag caggccacac |
| 7801 | cgtctcgagg gaggaggcca gatgcggcca gcgtctccaa cagggtgacc atccgctcgg |
| 7861 | cttgctgagc gtttaaacaa atgtttagac aggctgtggg gactcccctg agttgagcct |
| 7921 | tggccagggg tccggtgctg tcgcgggaaa cctccagcct tgttcttcaa accactcagc |
| 7981 | tcatgtgttt tgcactgact agtactgaat aatacaacca ctcttattta atgttagtat |
| 8041 | tatttatttg acaactcagt gtctaacagc ttgatatgca ggtccttgca tcctacattt |
| 8101 | ctttaggaag ttacccattt gtaactttaa aaacaggaaa aatatcagtt ggcaaatgca |
| 8161 | atcttttttt tttttaagct aaaggtgggt gaactggaat gaaaatcttt ctgatgttgt |
| 8221 | gtctataagc agccttgatg ggatatgtta gaagtgtcat gaaagtgtga ttctactttt |
| 8281 | gcagaaaaat ctaaagatca atttatatag ctttattttt tactttatca aagtatacag |
| 8341 | aattttaata tgcatatatt gtgtctgact taaaattata atgtctgcgt caccatttaa |
| 8401 | aatgtctgtt cattatgtaa tgtaataaaa gaaggtcttc aaaaatgtat ttaacatgaa |
| 8461 | tggtatccat agttgtcatc atcataaata ctggagttta tttttaaatt attaaacata |
| 8521 | gtaggtgcat taacataaat cagtctccac acagtaacat ttaactgata attcattaat |
| 8581 | cagctttgaa aaattaaatt gttaattaaa ccaatctaac atttcagtaa agtttatttt |
| 8641 | gtatgcttct gtttttaact tttatttctg tagataaact gactggataa tattatattg |
| 8701 | gacttttctc tagattatct aagcaggaga cctgaatctg cttgcaataa agaataaaag |
| 8761 | tctgcttcag tttctttata aagaaactca cacaa |

In some embodiments of the methods of the disclosure, the wild type human ATP11A gene of the disclosure consists of or comprises the nucleic acid sequence (Genbank Accession number: NP_056020.2, transcript variant 1):

| (SEQ ID NO: 26) | |
|---|---|
| 1 | mdcslvrtlv hrycageenw vdsrtiyvgh repppgaeay ipqrypdnri vsskytfwnf |
| 61 | ipknlfeqfr rvanfyflii flvqliidtp tspvtsglpl ffvitvtaik qgyedwlrhk |
| 121 | adnamnqcpv hfiqhgklvr kqsrklrvgd ivmvkedetf pcdliflssn rgdgtchvtt |
| 181 | asldgesshk thyavqdtkg fhteediggl hatieceqpq pdlykfvgri nvysdlndpv |
| 241 | vrplgsenll lrgatlknte kifgvaiytg metkmalnyq sksqkrsave ksmnaflivy |
| 301 | lciliskali ntvlkymwqs epfrdepwyn qkteserqrn lflkaftdfl afmvlfnyii |
| 361 | pvsmyvtvem qkflgsyfit wdedmfdeet gegplvntsd lneelgqvey iftdktgtlt |
| 421 | ennmefkecc ieghvyvphv icngqvlpes sgidmidssp svngrereel ffralclcht |
| 481 | vqvkdddsvd gprkspdggk scvyissspd evalvegvqr lgftylrlkd nymeilnren |
| 541 | hierfellei lsfdsvrrrm svivksatge iylfckgads sifprviegk vdqirarver |
| 601 | naveglrtlc vaykrliqee yegickllqa akvalqdrek klaeayeqie kdltllgata |
| 661 | vedrlqekaa dtiealqkag ikvwvltgdk metaaatcya cklfrrntql lelttkriee |
| 721 | qslhdvlfel sktvlrhsgs ltrdnlsgls admqdyglii dgaalslimk predgssgny |
| 781 | relfleicrs csavlccrma plqkaqivkl ikfskehpit laigdgandv smileahvgi |
| 841 | gvigkegrqa arnsdyaipk fkhlkkmllv hghfyyiris elvqyffykn vcfifpqfly |
| 901 | qffcgfsqqt lydtayltly nisftslpil lyslmeqhvg idvlkrdptl yrdvaknall |
| 961 | rwrvfiywtl lglfdalvff fgayfvfent tvtsngqifg nwtfgtlvft vmvftvtlkl |
| 1021 | aldthywtwi nhfviwgsll fyvvfsllwg gviwpflnyq rmyyvfiqml ssgpawlaiv |
| 1081 | llvtisllpd vlkkvlcrql wptatervqt ksqclsveqs tifmlsqtss slsf |

In some embodiments of the methods of the disclosure, the wild type human ATP11A gene of the disclosure consists of or comprises the nucleic acid sequence (Genbank Accession number: NM_032189.3, transcript variant 2):

| (SEQ ID NO: 48) | |
|---|---|
| 1 | gcggccgcac tagtaccccg gagcccatgg gcgcgccgag ccgggcgcgg gggcgctgaa |
| 61 | cggcggagcg ggagcggccg gaggagccat ggactgcagc ctcgtgcgga cgctcgtgca |
| 121 | cagatactgt gcaggagaag agaattgggt ggacagcagg accatctacg tgggacacag |
| 181 | ggagccacct ccgggcgcag aggcctacat cccacagaga tacccagaca acaggatcgt |
| 241 | ctcgtccaag tacacatttt ggaactttat acccaagaat ttatttgaac aattcagaag |
| 301 | agtagccaac ttttatttcc ttatcatatt tctggtgcag ttgattattg atacacccac |
| 361 | aagtccagtg acaagcggac ttccactctt ctttgtcatt actgtgacgg ctatcaaaca |
| 421 | gggttatgaa gactggcttc gacataaagc agacaatgcc atgaaccagt gtcctgttca |
| 481 | tttcattcag cacggcaagc tcgttcggaa acaaagtcga aagctgcgag ttggggacat |
| 541 | tgtcatggtt aaggaggacg agacctttcc ctgcgacttg atcttccttt ccagcaaccg |
| 601 | gggagatggg acgtgccacg tcaccaccgc cagcttggat ggagaatcca gccataaaac |
| 661 | gcattacgcg gtccaggaca ccaaaggctt ccacacagag gaggatatcg gcggacttca |
| 721 | cgccaccatc gagtgtgagc agccccagcc cgacctctac aagttcgtgg gtcgcatcaa |
| 781 | cgtttacagt gacctgaatg accccgtggt gaggccctta ggatcggaaa acctgctgct |
| 841 | tagaggagct acactgaaga acactgagaa aatctttggt gtggctattt acacgggaat |
| 901 | ggaaaccaag atggcattaa attatcaatc aaaatctcag aagcgatctg ccgtggaaaa |
| 961 | atcgatgaat gcgttcctca ttgtgtatct ctgcattctg atcagcaaag ccctgataaa |
| 1021 | cactgtgctg aaatacatgt ggcagagtga gccctttcgg gatgagccgt ggtataatca |
| 1081 | gaaaacggag tcggaaaggc agaggaatct gttcctcaag gcattcacgg acttcctggc |
| 1141 | cttcatggtc ctctttaact acatcatccc tgtgtccatg tacgtcacgg tcgagatgca |
| 1201 | gaagttcctc ggctcttact tcatcacctg ggacgaagac atgtttgacg aggagactgg |
| 1261 | cgaggggcct ctggtgaaca cgtcggacct caatgaagag ctgggacagg tggagtacat |
| 1321 | cttcacagac aagaccggca ccctcacgga aaacaacatg gagttcaagg agtgctgcat |
| 1381 | cgaaggccat gtctacgtgc cccacgtcat ctgcaacggg caggtcctcc cagagtcgtc |
| 1441 | aggaatcgac atgattgact cgtcccccag cgtcaacggg agggagcgcg aggagctgtt |
| 1501 | tttccgggcc ctctgtctct gccacaccgt ccaggtgaaa gacgatgaca gcgtagacgg |
| 1561 | ccccaggaaa tcgccggacg gggggaaatc ctgtgtgtac atctcatcct cgcccgacga |
| 1621 | ggtggcgctg gtcgaaggtg tccagagact tggctttacc tacctaaggc tgaaggacaa |
| 1681 | ttacatggag atattaaaca gggagaacca catcgaaagg tttgaattgc tggaaatttt |
| 1741 | gagttttgac tcagtcagaa ggagaatgag tgtaattgta aaatctgcta caggagaaat |
| 1801 | ttatctgttt tgcaaaggag cagattcttc gatattcccc cgagtgatag aaggcaaagt |
| 1861 | tgaccagatc cgagccagag tggagcgtaa cgcagtggag gggctccgaa ctttgtgtgt |
| 1921 | tgcttataaa aggctgatcc aagaagaata tgaaggcatt tgtaagctgc tgcaggctgc |
| 1981 | caaagtggcc cttcaagatc gagagaaaaa gttagcagaa gcctatgagc aaatagagaa |
| 2041 | agatcttact ctgcttggtg ctacagctgt tgaggaccgg ctgcaggaga aagctgcaga |
| 2101 | caccatcgag gccctgcaga aggccgggat caaagtctgg gttctcacgg gagacaagat |
| 2161 | ggagacggcc gcggccacgt gctacgcctg caagctcttc cgcaggaaca cgcagctgct |
| 2221 | ggagctgacc accaagagga tcgaggagca gagcctgcac gacgtcctgt tcgagctgag |
| 2281 | caagacggtc ctgcgccaca gcgggagcct gaccagagac aacctgtccg gactttcagc |
| 2341 | agatatgcag gactacggtt taattatcga cggagctgca ctgtctctga taatgaagcc |
| 2401 | tcgagaagac gggagttccg gcaactacag ggagctcttc ctggaaatct gccggagctg |
| 2461 | cagcgcggtg ctctgctgcc gcatggcgcc cttgcagaag gctcagattg ttaaattaat |
| 2521 | caaattttca aaagagcacc caatcacgtt agcaattggc gatggtgcaa atgatgtcag |
| 2581 | catgattctg gaagcgcacg tgggcatagg tgtcatcggc aaggaaggcc gccaggctgc |
| 2641 | caggaacagc gactatgcaa tcccaaagtt taagcatttg aagaagatgc tgcttgttca |
| 2701 | cgggcatttt tattacatta ggatctctga gctcgtgcag tacttcttct ataagaacgt |
| 2761 | ctgcttcatc ttccctcagt ttttatacca gttcttctgt gggttttcac aacagacttt |
| 2821 | gtacgacacc gcgtatctga ccctctacaa catcagcttc acctccctcc ccatcctcct |
| 2881 | gtacagcctc atggagcagc atgttggcat tgacgtgctc aagagagacc cgaccctgta |
| 2941 | cagggacgtc gccaagaatg ccctgctgcg ctggcgcgtg ttcatctact ggacgctcct |
| 3001 | gggactgttt gacgcactgg tgttcttctt tggtgcttat ttcgtgtttg aaaatacaac |
| 3061 | tgtgacaagc aacgggcaga tatttggaaa ctggacgttt ggaacgctgg tattcaccgt |
| 3121 | gatggtgttc acagttacac taaagcttgc attggacaca cactactgga cttggatcaa |
| 3181 | ccattttgtc atctgggggt cgctgctgtt ctacgttgtc ttttcgcttc tctggggagg |
| 3241 | agtgatctgg ccgttcctca actaccagag gatgtactac gtgttcatcc agatgctgtc |
| 3301 | cagcgggccc gcctggctgg ccatcgtgct gctggtgacc atcagcctcc ttcccgacgt |
| 3361 | cctcaagaaa gtcctgtgcc ggcagctgtg gccaacagca acagagagag tccagaatgg |
| 3421 | gtgcgcacag cctcgggacc gcgactcaga attcacccct cttgcctctc tgcagagccc |
| 3481 | aggctaccag agcacctgtc cctcggccgc ctggtacagc tcccactctc agcaggtgac |
| 3541 | actcgcggcc tggaaggaga aggtgtccac ggagccccca cccatcctcg gcggttccca |
| 3601 | tcaccactgc agttccatcc caagtcacag ctgccctagg tcccgtgtgg gaatgctcgt |
| 3661 | gtgatggatg gtcctaagcc tgtggagact gtgcacgtgc ctcttcctgg cccccagcag |
| 3721 | gcaaggaggg gggtcacagg ccttgccctc gagcatggca ccctggccgc ctggacccag |
| 3781 | cactgtggtt gttgagccac accagtggcc tctgggcatt cggctcaacg caggagggac |
| 3841 | attctgctgg cccaccctgc gcgctgtcat gcagaggcca ttcccccagg cctgtgtctt |
| 3901 | cacccacctg ccatcattgg cctttgctgt cactgggaga gaagagccgt ccagggaccc |
| 3961 | atggtggccc acatgtggat gccacatgct gctgtttcct gcttgcccgg ccaccaccca |
| 4021 | tgccctccat agggtgaggt ggagccatgg tggtgcgtcc tttactcaac aaccctccaa |
| 4081 | tccggatgct gtgggaaggg ccgggtcact cggataccat catccctgcg gatgcaccgc |
| 4141 | cgtaccctgc tcatctggga gtggtttccc tgcggttacg tccaagcccg cctgccctgt |
| 4201 | gtgttggggc tggctgagtt tcggtctccc catcaccggc cgcctcgtgg agaaggcagt |
| 4261 | gccacgtggg aggacaaggc cacgccggca gcttccagcc ctgccgcaga agtgccagga |
| 4321 | tgtccatcag ccactcgcca gggcacggag ccgtcagtcc actgttacgg gagaatgttg |
| 4381 | atttcgcggg tgcgagggcc gggagacaga tacttggctg tgatgagcag acatcctctg |
| 4441 | tccccgtgga ggggtcaaca ccaaggtggt gttcgtgcac cagaacctgt ctcgggctga |
| 4501 | cgggggtggc acacaggaca cgggtggatc ccaacaggca gcaccgcacc tctgcccgcc |
| 4561 | tcccgcactg cagctccgcc cgccgggctc tgcgtcccca cgtcccctcg tcccatcccc |
| 4621 | acgtcccctc atcccgtcac ctcgtcccca catccccttg ccccgtcacc tcgtcctcat |
| 4681 | gtccccttgt cctgtcacct cgtccccacg tcccctcgtc tcctcatccc cacgtcctct |
| 4741 | cgtccccttg tcccgtcccc acataccctc gtccccatgt ccccacgcag ggctctcctt |
| 4801 | cgtcttagga tctgtccagc gctgctctgg gtgggttagc aaccccaggg ctgctgtgat |
| 4861 | aggaagtccc tgttgttctc cgtactggca tttctatttc tagaaataat atttgacata |
| 4921 | gccttaatgg tccttaaaga agacatttca gtgtgagatt cagacttcag acgctgaaac |
| 4981 | tgctgccttt caggaaagca ccaccaacgc tggaggagga gccggccctc acgcccgccc |
| 5041 | cgcgccacgc tgtggaacgg ggctccggca agtgaaaccc agagggtgtt tccgaggtgc |
| 5101 | tcgacagtag gtatttttgg aagctcagat ttcaccattt gattgtataa tcttttacct |
| 5161 | ataaaatatt tatttgaagt agagggtaaa tcagcggtaa gaacagtgaa cacagtggtt |
| 5221 | gggataaaat aaggtgacaa acatcacacc aaagatgagg gtagcgagca actggcttga |
| 5281 | gcagacagaa cggggaagac tccactctgt cccgaggggc cagccgcagg cgtccccagg |
| 5341 | gccaccctgc cctgaggtcc ttgtgtggcc gccctggctt ggcagccctg cccacgctgc |
| 5401 | ccccgcaaac aatggtgtgt gcgtttttac agcccttttt aggaacccaa tatgggcata |
| 5461 | aatgtaacac ctgtagcggg ggcagattct ctgtatgttc agttaacaaa ttatttgtaa |
| 5521 | tgtatttttt tagaaatctt aaaattgcct ttgcactgaa gtattttcat agctgtttat |
| 5581 | atctctttta ttcatttatt taacatactg tctaatttta aaaataggtt tttaaagctt |
| 5641 | tcatttttaa gtttatgaaa ttttggccac tttacattta gattctggtg agagttttga |
| 5701 | ctgaatgttc caatctctga tgaatgcgaa ttttcagatt tgattttatt ctctacacac |
| 5761 | acctcttctt ttcttggtat ttctggtggc agtgattagt tgaacagcac atttaaggca |
| 5821 | cgataatttg ctacactttt tctttacaat ttgttgcaat ttcatctgct ttctatgttt |
| 5881 | cattgttaat tgccatcctt cagccttaaa aatagaagat tctcacgtga aggtttagta |
| 5941 | agttgggtcc cagctctgcc tgtgtggaga tagtcaccat gtacctctga caacaagttt |
| 6001 | tagtgtgaaa gtcactaaac ttttacacac tcccaaacgt ctttttaaaa attgcttggg |
| 6061 | aaattattaa atgaatgtgc ctgatgattt gaaatagaca aggggcacga gataaaaaag |
| 6121 | aaaaggatga gaagatcctc agtgaatgac gttgcagggt cttcatgcaa ttttccacct |
| 6181 | cgcagtagtt agtatttact tgccttaaac taactttgaa gcaagtaatg tcaactttga |
| 6241 | gcactttgtt gagttttgaa aaatcttatt tgttgctgca caggttaata aattatcaat |
| 6301 | ttgtaattca gcatgttggt cagagacacg gtcactgatt cacacccagt ccctgccaca |
| 6361 | gaccgtctca gacacgcaca gtgggcctgc tgcatgattc acacccagtc cctgccacag |
| 6421 | accgtctcag acacgcacag tgggcctgct gcatgattca cacccagtcc ctgccacaga |
| 6481 | ccgtctcaga cacgcacagt gggcctgctg catgcgtgtt acctggcttt tggctccacg |
| 6541 | ctcactcata gccatgtcca catgggggct tgcacacagg atcactcaca tatgtacatg |
| 6601 | tacccaccac aaacgtgcaa gctcctgcac acatgcatgc acacaaacgt gtacacaagt |
| 6661 | gtgagctcct acacgcatac acacacacac gtgtacatgc accaaagcat gtgtgaccta |
| 6721 | cagacatgca gaacatgcac gtgtacacat accacagaca cgcgtgtgca tgctcctaca |
| 6781 | caatacatat gcacatatca tgaacagcgt aagttcctac acacggacgt gtgatacaca |
| 6841 | catgcatgta caggtaagca cacatgtaca agctcctaca ggcttgctct cacacacgtg |
| 6901 | tatgcacagc agagagacgt atgagcttct actgcacaca tgcacacaca cacgcacacg |
| 6961 | tacattcact acaaacgtgc agcctcctgc acacgtgcac attcatgtgt acaccacaaa |
| 7021 | tgagttccca gacgtgtaaa cacacgtgca cacatcgtac acatgtgagc tcccacacgt |
| 7081 | acacacagat gcacatggac acaccccaaa cacgcacagg ctcctacaca catgcacaca |
| 7141 | cgtgtacacc acaaacgagc tcccagacat gtaaacacac gtctcccaca cgtgagctcc |
| 7201 | cacacgtaca catgcacatg tacgcaccac aaacacatgc gcaggctcct gcaggcgtga |
| 7261 | atacacacat gcacacacat atacacacat gtgccacaaa caagtgcaca ctgtcctggt |
| 7321 | gtcctgcact gcatcctgcc tccttgctga ggggcccctg tgagaggcct ctggatgggc |
| 7381 | atgggaagat gggctccctg gcccccagcc catgcctccc tgggatgaag agtccccctc |
| 7441 | ctggcagaat gtctgggctt tgcagagcag gccccggggg tgaagtcgca gcttcactta |
| 7501 | caccagctgc tctgtgagca aggcttggtg ccctggacaa ggcccttccc ctttagggag |
| 7561 | gtccagcctc gcaagctgaa acctcccctc ggctcagccc tataccaggc ggccacagca |
| 7621 | ggactggcca cacccacgcc gcacctcatc cgtgcacgcg tcggagcacg gccagccttc |
| 7681 | cgccacgagc cagctgggaa gggccgcggc cgcctaaagc cccagtcaac ccagcctgtg |
| 7741 | tctgagcaga cagggcgaac aagcaggcca caccgtctcg agggaggagg ccagatgcgg |
| 7801 | ccagcgtctc caacagggtg accatccgct cggcttgctg agcgtttaaa caaatgttta |
| 7861 | gacaggctgt ggggactccc ctgagttgag ccttggccag gggtccggtg ctgtcgcggg |
| 7921 | aaacctccag ccttgttctt caaaccactc agctcatgtg ttttgcactg actagtactg |
| 7981 | aataatacaa ccactcttat ttaatgttag tattatttat ttgacaactc agtgtctaac |
| 8041 | agcttgatat gcaggtcctt gcatcctaca tttctttagg aagttaccca tttgtaactt |
| 8101 | taaaaacagg aaaaatatca gttggcaaat gcaatctttt ttttttttaa gctaaaggtg |
| 8161 | ggtgaactgg aatgaaaatc tttctgatgt tgtgtctata agcagccttg atgggatatg |
| 8221 | ttagaagtgt catgaaagtg tgattctact tttgcagaaa aatctaaaga tcaatttata |
| 8281 | tagctttatt ttttacttta tcaaagtata cagaatttta atatgcatat attgtgtctg |
| 8341 | acttaaaatt ataatgtctg cgtcaccatt taaaatgtct gttcattatg taatgtaata |
| 8401 | aaagaaggtc ttcaaaaatg tatttaacat gaatggtatc catagttgtc atcatcataa |
| 8461 | atactggagt ttatttttaa attattaaac atagtaggtg cattaacata aatcagtctc |
| 8521 | cacacagtaa catttaactg ataattcatt aatcagcttt gaaaaattaa attgttaatt |
| 8581 | aaaccaatct aacatttcag taaagtttat tttgtatgct tctgttttta acttttattt |
| 8641 | ctgtagataa actgactgga taatattata ttggactttt ctctagatta tctaagcagg |
| 8701 | agacctgaat ctgcttgcaa taaagaataa aagtctgctt cagtttcttt ataaagaaac |
| 8761 | tcacacaa |

In some embodiments of the methods of the disclosure, the wild type human ATP11A gene of the disclosure consists of or comprises the amino acid sequence (Genbank Accession number: NP_115565.3, transcript variant 2):

| (SEQ ID NO: 49) | |
|---|---|
| 1 | mdcslvrtlv hrycageenw vdsrtiyvgh repppgaeay ipqrypdnri vsskytfwnf |
| 61 | ipknlfeqfr rvanfyflii flvqliidtp tspvtsglpl ffvitvtaik qgyedwlrhk |
| 121 | adnamnqcpv hfiqhgklvr kqsrklrvgd ivmvkedetf pcdliflssn rgdgtchvtt |
| 181 | asldgesshk thyavqdtkg fhteediggl hatieceqpq pdlykfvgri nvysdlndpv |
| 241 | vrplgsenll lrgatlknte kifgvaiytg metkmalnyq sksqkrsave ksmnaflivy |
| 301 | lciliskali ntvlkymwqs epfrdepwyn qkteserqrn lflkaftdfl afmvlfnyii |
| 361 | pvsmyvtvem qkflgsyfit wdedmfdeet gegplvntsd lneelgqvey iftdktgtlt |
| 421 | ennmefkecc ieghvyvphv icngqvlpes sgidmidssp svngrereel ffralclcht |
| 481 | vqvkdddsvd gprkspdggk scvyissspd evalvegvqr lgftylrlkd nymeilnren |
| 541 | hierfellei lsfdsvrrrm svivksatge iylfckgads sifprviegk vdqirarver |
| 601 | naveglrtlc vaykrliqee yegickllqa akvalqdrek klaeayeqie kdltllgata |
| 661 | vedrlqekaa dtiealqkag ikvwvltgdk metaaatcya cklfrrntql lelttkriee |
| 721 | qslhdvlfel sktvlrhsgs ltrdnlsgls admqdyglii dgaalslimk predgssgny |
| 781 | relfleicrs csavlccrma plqkaqivkl ikfskehpit laigdgandv smileahvgi |
| 841 | gvigkegrqa arnsdyaipk fkhlkkmllv hghfyyiris elvqyffykn vcfifpqfly |
| 901 | qffcgfsqqt lydtayltly nisftslpil lyslmeqhvg idvlkrdptl yrdvaknall |
| 961 | rwrvfiywtl lglfdalvff fgayfvfent tvtsngqifg nwtfgtlvft vmvftvtlkl |
| 1021 | aldthywtwi nhfviwgsll fyvvfsllwg gviwpflnyq rmyyvfiqml ssgpawlaiv |
| 1081 | llvtisllpd vlkkvlcrql wptatervqn gcaqprdrds eftplaslqs pgyqstcpsa |
| 1141 | awysshsqqv tlaawkekvs tepppilggs hhhcssipsh scprsrvgml v |

In some embodiments of the methods of the disclosure, the wild type human IVD/DISP2 gene of the disclosure consists of or comprises the nucleic acid sequence (Genbank Accession number: NM_002225.3, transcript variant 1):

| (SEQ ID NO: 50) | |
|---|---|
| 1 | tttccgcagt taggggctgc tatttcaacg cagggagata aaaagaaaaa aacacttgct |
| 61 | cttctacccc gctaaaaaca ctcatcctag ggagcacgcc agcatttgca gcgttcgggg |
| 121 | cagggccact cggcctgcgg ccgttgcact ggctggaagc tggcaggcga tcacggttga |
| 181 | ttggctcggg tgcggtccaa gggcagcaac gccttcggcg ggccgcctag ggtgattggc |
| 241 | tgctgcagcc caccccctag ccggtttggt gggcggcgaa gcctggattg gtggagctaa |
| 301 | gagctggctc agtttcagcg ctggctcttc gtgcatggca gagatggcga ctgcgactcg |
| 361 | gctgctgggg tggcgtgtgg cgagctggag gctgcggccg ccgcttgccg gcttcgtttc |
| 421 | ccagcgggcc cactcgcttt tgcccgtgga cgatgcaatc aatgggctaa gcgaggagca |
| 481 | gaggcagctt cgtcagacca tggctaagtt ccttcaggag cacctggccc ccaaggccca |
| 541 | ggagatcgat cgcagcaatg agttcaagaa cctgcgagaa ttttggaagc agctggggaa |
| 601 | cctgggcgta ttgggcatca cagcccctgt tcagtatggc ggctccggcc tgggctacct |
| 661 | ggagcatgtg ctggtgatgg aggagatatc ccgagcttcc ggagcagtgg ggctcagtta |
| 721 | cggtgcccac tccaacctct gcatcaacca gcttgtacgc aatgggaatg aggcccagaa |
| 781 | agagaagtat ctcccgaagc tgatcagtgg tgagtacatc ggagccctgg ccatgagtga |
| 841 | gcccaatgca ggctctgatg ttgtctctat gaagctcaaa gcggaaaaga aaggaaatca |
| 901 | ctacatcctg aatggcaaca agttctggat cactaatggc cctgatgctg acgtcctgat |
| 961 | tgtctatgcc aagacagatc tggctgctgt gccagcttct cggggcatca cagccttcat |
| 1021 | tgtggagaag ggtatgcctg gctttagcac ctctaagaag ctggacaagc tggggatgag |
| 1081 | gggctctaac acctgtgagc taatctttga agactgcaag attcctgctg ccaacatcct |
| 1141 | gggccatgag aataagggtg tctacgtgct gatgagtggg ctggacctgg agcggctggt |
| 1201 | gctggccggg gggcctcttg ggctcatgca agcggtcctg gaccacacca ttccctacct |
| 1261 | gcacgtgagg gaagcctttg gccagaagat cggccacttc cagttgatgc aggggaagat |
| 1321 | ggctgacatg tacacccgcc tcatggcgtg tcggcagtat gtctacaatg tcgccaaggc |
| 1381 | ctgcgatgag ggccattgca ctgctaagga ctgtgcaggt gtgattcttt actcagctga |
| 1441 | gtgtgccaca caggtagccc tggacggcat tcagtgtttt ggtggcaatg gctacatcaa |
| 1501 | tgactttccc atgggccgct ttcttcgaga tgccaagctg tatgagatag gggctgggac |
| 1561 | cagcgaggtg aggcggctgg tcatcggcag agccttcaat gcagactttc actagtcctg |
| 1621 | agacccttcg cccccttttc ctgcacctag tggcctttct tgggaagtag agatgtggcg |
| 1681 | gctttcccac cctgcccaca gcaggccctc ctgcccagct gctcttgtca gccctctggc |
| 1741 | ctctggatga ggttgagttc tccacaacag ctcccaagca tcatgggcct cgcagccggg |
| 1801 | cctgtgccac ggctagtgtt gtgtgattta aaatggactc agcaggaagc atattgtctg |
| 1861 | gggattgttg ggacaggttt tggtgactct gtgcccttgc tctctaactt ctgagcccac |
| 1921 | ctcccagggt aggcacctgg gggcatgcag gtgcccacct cccagggtag gcacctgggg |
| 1981 | gcatgcaggt acccacctct ttctcttggg tgaggctctg gcaaggagat ctctctgctc |
| 2041 | aagcacagca gaatcatggc ccctctccat gaattggaac ttggtacagg ttaagtatcc |
| 2101 | ctaatcctga aatctgaaac acttgtggtt ccaagcattt tggataaggc aaattcaact |
| 2161 | ttcagtctct tttctggggg aaaaaaataa taaacctagc ctagccaggc gtggtggctc |
| 2221 | atgcttgtaa tcccagcact tcaggaggct gagatgggtg gatcacctga ggtcaggagt |
| 2281 | tcaagaccag cctggccaac atgtggaaac ctcgcctcaa ctaaaaatag aaaaaaatta |
| 2341 | gttgggcatg gtggtgggca cctgtaatcc cagctacttc aggaggctga ggcaggagaa |
| 2401 | ttacttgaac ccaggaggcg gacgttgcag tgagccgagc ttgtgccatt gcactccagc |
| 2461 | ctgggcgaca agagcaaaac tcttcaaaaa acaaaacaaa acaaaaaaac cctggccctt |
| 2521 | gtttcttcca gtttctagag gtatcagctc ctagcagctt atgaacacat atgcttgctt |
| 2581 | ggccaggcaa ggtggtgtgt gcctgtaatc ccagcacttt gggaggccaa ggcaggtgga |
| 2641 | tcacttgcag tcaggagttc aagaccagcc tgtccaacgt ggtgaaaccc catctctact |
| 2701 | aaaaatacaa aaattagcca ggggtggtgg tgcacgtctg taatcccagc tactcaggag |
| 2761 | gctgaggcag gagaatcact tgaacccggg aggtggaggt tgcaatgagc caatatgaca |
| 2821 | ccgctgcagt ccagcctggg ccatagagtg agactctgtc tcaaaaaagg aaagaaaaat |
| 2881 | aggctgggca cagtgactca tgcctgtaat cccaacactt tgggaggccg aggcaggtgg |
| 2941 | atcacgaggt caggagttca agaccagcct ggccaagatg gtaaaacctc gtctctacta |
| 3001 | aaaatacaaa aattagccag gtgtggtggc aggctcctgt aatcccagct actcaggagg |
| 3061 | ctgaggcaga gaattgcttg aacccgggag gcagagtttg cagtgagcca agatcacacc |
| 3121 | actgcactcc agcttggacg acagagcgag actctgtctc aaaaaataat aggccaggca |
| 3181 | tggtggctca acgtctgtaa tcccagcact ttgggaggcc gaggcgggca gatcacaagg |
| 3241 | tcaggagttc gagaccagcc tgacgaccaa catggtgaaa cctcgtctct actaaaaata |
| 3301 | caaaaattag ccaggcctgg tggcacgcgc ctgtaatccc agttacacag aagactgagg |
| 3361 | caggagaatc gcttgaacgc aggaggcaga ggttgcagga gctgagatcg cgccattgca |
| 3421 | ctccagcctg ggcaacagag tgagactctg tctcaaaaaa taataataaa ataaatgaac |
| 3481 | acacatgctg ctgagtccgc agggggggca gagcagagga cagcgtgctt ttgtgtactg |
| 3541 | ttggaagact ggctcctcct gtacagcacc tctgagccct tgtgcaccgc cctgccacgg |
| 3601 | gcaccatcca gtcctggccg tgtgaccacc cacagctgac tgggcagcag gcacaggccc |
| 3661 | tacccgagca ggccggagtt ggctcgcatg actccagctg aggctgcctg tgtacatttc |
| 3721 | tccagatacc ctatggctaa ttttgttata actgcacagt ggctgctgcc attttgtatt |
| 3781 | aaatatattg tgaaacaaac ctatctgggg agaagcaatc tacttgccgc tgcttcctgt |
| 3841 | ctggatccag cttgtgtcct tggagagtgg ctggcccagg tcctattcct gtcctccagc |
| 3901 | ccgttctttc atgagggaca ggaaggtaaa atcagccctt aggagagagg tctcagcctc |
| 3961 | cctttcccag atctcccagt gagttttaaa ggaagcaggg agcccagagt gctaagttct |
| 4021 | tacagccaga aggaagctta tagatttctg aaaaccgccc ctttgttttt aaaaagatca |
| 4081 | acacaatttg actttctcaa ggtcaaaacg aactagaatc cagatctgct catggcaaaa |
| 4141 | atgggggtgt tctgagaatt ccagctttgg gccgcactgt acagcagtct ggatagagtg |
| 4201 | tgatctgaga agggaatggg tctgggttgt tccacccctt ccgagttcca aaaagaggga |
| 4261 | actggttttc ttggttctca gcccagcagc acctatcctg gctcttggtc ctggcctgca |
| 4321 | gccaagtgct gttcctagcc tgaggcttga gacaggtggg gttggctcct caccaacccc |
| 4381 | agttccgtcc catcctgagg gcaagatcct gggctcatag gcagtccctt tcacttcctt |
| 4441 | gtcttgctcc ctgctatgtt ggagatgaat gtgactaaaa gggccatctt gctggcttaa |
| 4501 | tgtgtggctg gagagaccag cctggagaca atgtggcaaa atggggcgct tcatccagtc |
| 4561 | tgtctaagcc ctgtcgactt ggggaggtga tttctttcct ggttctatat gtgaagcaaa |
| 4621 | ataaatgttt taaaattaaa agcaaaaaaa acaaaatgaa ccatgaaaaa aaa |

In some embodiments of the methods of the disclosure, the wild type human IVD/DISP2 gene of the disclosure consists of or comprises the amino acid sequence (Genbank Accession number: transcript variant 1):

| (SEQ ID NO: 51) | |
|---|---|
| 1 | maematatrl lgwrvaswrl rpplagfvsq rahsllpvdd ainglseeqr qlrqtmakfl |
| 61 | qehlapkage idrsnefknl refwkqlgnl gvlgitapvq yggsglgyle hvlvmeeisr |
| 121 | asgavglsyg ahsnlcinql vrngneaqke kylpklisge yigalamsep nagsdvvsmk |
| 181 | lkaekkgnhy ilngnkfwit ngpdadvliv yaktdlaavp asrgitafiv ekgmpgfsts |
| 241 | kkldklgmrg sntcelifed ckipaanilg henkgvyvlm sgldlerlvl aggplglmqa |
| 301 | vldhtipylh vreafgqkig hfqlmqgkma dmytrlmacr qyvynvakac deghctakdc |
| 361 | agvilysaec atqvaldgiq cfggngyind fpmgrflrda klyeigagts evrrlvigra |
| 421 | fnadfh |

In some embodiments of the methods of the disclosure, the wild type human IVD/DISP2 gene of the disclosure consists of or comprises the nucleic acid sequence (Genbank Accession number: NM_001159508.1, transcript variant 2):

| (SEQ ID NO: 27) | |
|---|---|
| 1 | tttccgcagt taggggctgc tatttcaacg cagggagata aaaagaaaaa aacacttgct |
| 61 | cttctacccc gctaaaaaca ctcatcctag ggagcacgcc agcatttgca gcgttcgggg |
| 121 | cagggccact cggcctgcgg ccgttgcact ggctggaagc tggcaggcga tcacggttga |
| 181 | ttggctcggg tgcggtccaa gggcagcaac gccttcggcg ggccgcctag ggtgattggc |
| 241 | tgctgcagcc caccccctag ccggtttggt gggcggcgaa gcctggattg gtggagctaa |
| 301 | gagctggctc agtttcagcg ctggctcttc gtgcatggca gagatggcga ctgcgactcg |
| 361 | gctgctgggg tggcgtgtgg cgagctggag gctgcggccg ccgcttgccg gcttcgtttc |
| 421 | ccagcgggcc cactcgcttt tgcccgtgga cgatgcaatc aatgggctaa gcgaggagca |
| 481 | gaggcaggaa ttttggaagc agctggggaa cctgggcgta ttgggcatca cagcccctgt |
| 541 | tcagtatggc ggctccggcc tgggctacct ggagcatgtg ctggtgatgg aggagatatc |
| 601 | ccgagcttcc ggagcagtgg ggctcagtta cggtgcccac tccaacctct gcatcaacca |
| 661 | gcttgtacgc aatgggaatg aggcccagaa agagaagtat ctcccgaagc tgatcagtgg |
| 721 | tgagtacatc ggagccctgg ccatgagtga gcccaatgca ggctctgatg ttgtctctat |
| 781 | gaagctcaaa gcggaaaaga aaggaaatca ctacatcctg aatggcaaca agttctggat |
| 841 | cactaatggc cctgatgctg acgtcctgat tgtctatgcc aagacagatc tggctgctgt |
| 901 | gccagcttct cggggcatca cagccttcat tgtggagaag ggtatgcctg gctttagcac |
| 961 | ctctaagaag ctggacaagc tggggatgag gggctctaac acctgtgagc taatctttga |
| 1021 | agactgcaag attcctgctg ccaacatcct gggccatgag aataagggtg tctacgtgct |
| 1081 | gatgagtggg ctggacctgg agcggctggt gctggccggg gggcctcttg ggctcatgca |
| 1141 | agcggtcctg gaccacacca ttccctacct gcacgtgagg gaagcctttg gccagaagat |
| 1201 | cggccacttc cagttgatgc aggggaagat ggctgacatg tacacccgcc tcatggcgtg |
| 1261 | tcggcagtat gtctacaatg tcgccaaggc ctgcgatgag ggccattgca ctgctaagga |
| 1321 | ctgtgcaggt gtgattcttt actcagctga gtgtgccaca caggtagccc tggacggcat |
| 1381 | tcagtgtttt ggtggcaatg gctacatcaa tgactttccc atgggccgct ttcttcgaga |
| 1441 | tgccaagctg tatgagatag gggctgggac cagcgaggtg aggcggctgg tcatcggcag |
| 1501 | agccttcaat gcagactttc actagtcctg agacccttcg cccccttttc ctgcacctag |
| 1561 | tggcctttct tgggaagtag agatgtggcg gctttcccac cctgcccaca gcaggccctc |
| 1621 | ctgcccagct gctcttgtca gccctctggc ctctggatga ggttgagttc tccacaacag |
| 1681 | ctcccaagca tcatgggcct cgcagccggg cctgtgccac ggctagtgtt gtgtgattta |
| 1741 | aaatggactc agcaggaagc atattgtctg gggattgttg ggacaggttt tggtgactct |
| 1801 | gtgcccttgc tctctaactt ctgagcccac ctcccagggt aggcacctgg gggcatgcag |
| 1861 | gtgcccacct cccagggtag gcacctgggg gcatgcaggt acccacctct ttctcttggg |
| 1921 | tgaggctctg gcaaggagat ctctctgctc aagcacagca gaatcatggc ccctctccat |
| 1981 | gaattggaac ttggtacagg ttaagtatcc ctaatcctga aatctgaaac acttgtggtt |
| 2041 | ccaagcattt tggataaggc aaattcaact ttcagtctct tttctggggg aaaaaaataa |
| 2101 | taaacctagc ctagccaggc gtggtggctc atgcttgtaa tcccagcact tcaggaggct |
| 2161 | gagatgggtg gatcacctga ggtcaggagt tcaagaccag cctggccaac atgtggaaac |
| 2221 | ctcgcctcaa ctaaaaatag aaaaaaatta gttgggcatg gtggtgggca cctgtaatcc |
| 2281 | cagctacttc aggaggctga ggcaggagaa ttacttgaac ccaggaggcg gacgttgcag |
| 2341 | tgagccgagc ttgtgccatt gcactccagc ctgggcgaca agagcaaaac tcttcaaaaa |
| 2401 | acaaaacaaa acaaaaaaac cctggccctt gtttcttcca gtttctagag gtatcagctc |
| 2461 | ctagcagctt atgaacacat atgcttgctt ggccaggcaa ggtggtgtgt gcctgtaatc |
| 2521 | ccagcacttt gggaggccaa ggcaggtgga tcacttgcag tcaggagttc aagaccagcc |
| 2581 | tgtccaacgt ggtgaaaccc catctctact aaaaatacaa aaattagcca ggggtggtgg |
| 2641 | tgcacgtctg taatcccagc tactcaggag gctgaggcag gagaatcact tgaacccggg |
| 2701 | aggtggaggt tgcaatgagc caatatgaca ccgctgcagt ccagcctggg ccatagagtg |
| 2761 | agactctgtc tcaaaaaagg aaagaaaaat aggctgggca cagtgactca tgcctgtaat |
| 2821 | cccaacactt tgggaggccg aggcaggtgg atcacgaggt caggagttca agaccagcct |
| 2881 | ggccaagatg gtaaaacctc gtctctacta aaaatacaaa aattagccag gtgtggtggc |
| 2941 | aggctcctgt aatcccagct actcaggagg ctgaggcaga gaattgcttg aacccgggag |
| 3001 | gcagagtttg cagtgagcca agatcacacc actgcactcc agcttggacg acagagcgag |
| 3061 | actctgtctc aaaaaataat aggccaggca tggtggctca acgtctgtaa tcccagcact |
| 3121 | ttgggaggcc gaggcgggca gatcacaagg tcaggagttc gagaccagcc tgacgaccaa |
| 3181 | catggtgaaa cctcgtctct actaaaaata caaaaattag ccaggcctgg tggcacgcgc |
| 3241 | ctgtaatccc agttacacag aagactgagg caggagaatc gcttgaacgc aggaggcaga |
| 3301 | ggttgcagga gctgagatcg cgccattgca ctccagcctg ggcaacagag tgagactctg |
| 3361 | tctcaaaaaa taataataaa ataaatgaac acacatgctg ctgagtccgc agggggggca |
| 3421 | gagcagagga cagcgtgctt ttgtgtactg ttggaagact ggctcctcct gtacagcacc |
| 3481 | tctgagccct tgtgcaccgc cctgccacgg gcaccatcca gtcctggccg tgtgaccacc |
| 3541 | cacagctgac tgggcagcag gcacaggccc tacccgagca ggccggagtt ggctcgcatg |
| 3601 | actccagctg aggctgcctg tgtacatttc tccagatacc ctatggctaa ttttgttata |
| 3661 | actgcacagt ggctgctgcc attttgtatt aaatatattg tgaaacaaac ctatctgggg |
| 3721 | agaagcaatc tacttgccgc tgcttcctgt ctggatccag cttgtgtcct tggagagtgg |
| 3781 | ctggcccagg tcctattcct gtcctccagc ccgttctttc atgagggaca ggaaggtaaa |
| 3841 | atcagccctt aggagagagg tctcagcctc cctttcccag atctcccagt gagttttaaa |
| 3901 | ggaagcaggg agcccagagt gctaagttct tacagccaga aggaagctta tagatttctg |
| 3961 | aaaaccgccc ctttgttttt aaaaagatca acacaatttg actttctcaa ggtcaaaacg |
| 4021 | aactagaatc cagatctgct catggcaaaa atgggggtgt tctgagaatt ccagctttgg |
| 4081 | gccgcactgt acagcagtct ggatagagtg tgatctgaga agggaatggg tctgggttgt |
| 4141 | tccacccctt ccgagttcca aaaagaggga actggttttc ttggttctca gcccagcagc |
| 4201 | acctatcctg gctcttggtc ctggcctgca gccaagtgct gttcctagcc tgaggcttga |
| 4261 | gacaggtggg gttggctcct caccaacccc agttccgtcc catcctgagg gcaagatcct |
| 4321 | gggctcatag gcagtccctt tcacttcctt gtcttgctcc ctgctatgtt ggagatgaat |
| 4381 | gtgactaaaa gggccatctt gctggcttaa tgtgtggctg gagagaccag cctggagaca |
| 4441 | atgtggcaaa atggggcgct tcatccagtc tgtctaagcc ctgtcgactt ggggaggtga |
| 4501 | tttctttcct ggttctatat gtgaagcaaa ataaatgttt taaaattaaa agcaaaaaaa |
| 4561 | acaaaatgaa ccatg |

In some embodiments of the methods of the disclosure, the wild type human IVD/DISP2 gene of the disclosure consists of or comprises the nucleic acid sequence (Genbank Accession number: NP 001152980.1, transcript variant 2):

| (SEQ ID NO: 28) | |
|---|---|
| 1 | maematatrl lgwrvaswrl rpplagfvsq rahsllpvdd ainglseeqr qefwkqlgnl |
| 61 | gvlgitapvq yggsglgyle hvlvmeeisr asgavglsyg ahsnlcinql vrngneaqke |
| 121 | kylpklisge yigalamsep nagsdvvsmk lkaekkgnhy ilngnkfwit ngpdadvliv |
| 181 | yaktdlaavp asrgitafiv ekgmpgfsts kkldklgmrg sntcelifed ckipaanilg |
| 241 | henkgvyvlm sgldlerlvl aggplglmqa vldhtipylh vreafgqkig hfqlmqgkma |
| 301 | dmytrlmacr qyvynvakac deghctakdc agvilysaec atqvaldgiq cfggngyind |
| 361 | fpmgrflrda klyeigagts evrrlvigra fnadfh |

In some embodiments of the methods of the disclosure, the wild type human DPP9 gene of the disclosure consists of or comprises the nucleic acid sequence (Genbank Accession number: NM_139159.4):

| (SEQ ID NO: 29) | |
|---|---|
| 1 | caacttccgg gtcaaaggtg cctgagccgg cgggtcccct gtgtccgccg cggctgtcgt |
| 61 | cccccgctcc cgccacttcc ggggtcgcag tcccgggcat ggagccgcga ccgtgaggcg |
| 121 | ccgctggacc cgggacgacc tgcccagtcc ggccgccgcc ccacgtcccg gtctgtgtcc |
| 181 | cacgcctgca gctggaatgg aggctctctg gaccctttag aaggcacccc tgccctcctg |
| 241 | aggtcagctg agcggttaat gcggaaggtt aagaaactgc gcctggacaa ggagaacacc |
| 301 | ggaagttgga gaagcttctc gctgaattcc gagggggctg agaggatggc caccaccggg |
| 361 | accccaacgg ccgaccgagg cgacgcagcc gccacagatg acccggccgc ccgcttccag |
| 421 | gtgcagaagc actcgtggga cgggctccgg agcatcatcc acggcagccg caagtactcg |
| 481 | ggcctcattg tcaacaaggc gccccacgac ttccagtttg tgcagaagac ggatgagtct |
| 541 | gggccccact cccaccgcct ctactacctg ggaatgccat atggcagccg agagaactcc |
| 601 | ctcctctact ctgagattcc caagaaggtc cggaaagagg ctctgctgct cctgtcctgg |
| 661 | aagcagatgc tggatcattt ccaggccacg ccccaccatg gggtctactc tcgggaggag |
| 721 | gagctgctga gggagcggaa acgcctgggg gtcttcggca tcacctccta cgacttccac |
| 781 | agcgagagtg gcctcttcct cttccaggcc agcaacagcc tcttccactg ccgcgacggc |
| 841 | ggcaagaacg gcttcatggt gtcccctatg aaaccgctgg aaatcaagac ccagtgctca |
| 901 | gggccccgga tggaccccaa aatctgccct gccgaccctg ccttcttctc cttcatcaat |
| 961 | aacagcgacc tgtgggtggc caacatcgag acaggcgagg agcggcggct gaccttctgc |
| 1021 | caccaaggtt tatccaatgt cctggatgac cccaagtctg cgggtgtggc caccttcgtc |
| 1081 | atacaggaag agttcgaccg cttcactggg tactggtggt gccccacagc ctcctgggaa |
| 1141 | ggttcagagg gcctcaagac gctgcgaatc ctgtatgagg aagtcgatga gtccgaggtg |
| 1201 | gaggtcattc acgtcccctc tcctgcgcta gaagaaagga agacggactc gtatcggtac |
| 1261 | cccaggacag gcagcaagaa tcccaagatt gccttgaaac tggctgagtt ccagactgac |
| 1321 | agccagggca agatcgtctc gacccaggag aaggagctgg tgcagccctt cagctcgctg |
| 1381 | ttcccgaagg tggagtacat cgccagggcc gggtggaccc gggatggcaa atacgcctgg |
| 1441 | gccatgttcc tggaccggcc ccagcagtgg ctccagctcg tcctcctccc cccggccctg |
| 1501 | ttcatcccga gcacagagaa tgaggagcag cggctagcct ctgccagagc tgtccccagg |
| 1561 | aatgtccagc cgtatgtggt gtacgaggag gtcaccaacg tctggatcaa tgttcatgac |
| 1621 | atcttctatc ccttccccca atcagaggga gaggacgagc tctgctttct ccgcgccaat |
| 1681 | gaatgcaaga ccggcttctg ccatttgtac aaagtcaccg ccgttttaaa atcccagggc |
| 1741 | tacgattgga gtgagccctt cagccccggg gaagatgaat ttaagtgccc cattaaggaa |
| 1801 | gagattgctc tgaccagcgg tgaatgggag gttttggcga ggcacggctc caagatctgg |
| 1861 | gtcaatgagg agaccaagct ggtgtacttc cagggcacca aggacacgcc gctggagcac |
| 1921 | cacctctacg tggtcagcta tgaggcggcc ggcgagatcg tacgcctcac cacgcccggc |
| 1981 | ttctcccata gctgctccat gagccagaac ttcgacatgt tcgtcagcca ctacagcagc |
| 2041 | gtgagcacgc cgccctgcgt gcacgtctac aagctgagcg gccccgacga cgaccccctg |
| 2101 | cacaagcagc cccgcttctg ggctagcatg atggaggcag ccagctgccc cccggattat |
| 2161 | gttcctccag agatcttcca tttccacacg cgctcggatg tgcggctcta cggcatgatc |
| 2221 | tacaagcccc acgccttgca gccagggaag aagcacccca ccgtcctctt tgtatatgga |
| 2281 | ggcccccagg tgcagctggt gaataactcc ttcaaaggca tcaagtactt gcggctcaac |
| 2341 | acactggcct ccctgggcta cgccgtggtt gtgattgacg gcaggggctc ctgtcagcga |
| 2401 | gggcttcggt tcgaaggggc cctgaaaaac caaatgggcc aggtggagat cgaggaccag |
| 2461 | gtggagggcc tgcagttcgt ggccgagaag tatggcttca tcgacctgag ccgagttgcc |
| 2521 | atccatggct ggtcctacgg gggcttcctc tcgctcatgg ggctaatcca caagccccag |
| 2581 | gtgttcaagg tggccatcgc gggtgccccg gtcaccgtct ggatggccta cgacacaggg |
| 2641 | tacactgagc gctacatgga cgtccctgag aacaaccagc acggctatga ggcgggttcc |
| 2701 | gtggccctgc acgtggagaa gctgcccaat gagcccaacc gcttgcttat cctccacggc |
| 2761 | ttcctggacg aaaacgtgca ctttttccac acaaacttcc tcgtctccca actgatccga |
| 2821 | gcagggaaac cttaccagct ccagatctac cccaacgaga gacacagtat tcgctgcccc |
| 2881 | gagtcgggcg agcactatga agtcacgttg ctgcactttc tacaggaata cctctgagcc |
| 2941 | tgcccaccgg gagccgccac atcacagcac aagtggctgc agcctccgcg gggaaccagg |
| 3001 | cgggagggac tgagtggccc gcgggcccca gtgaggcact ttgtcccgcc cagcgctggc |
| 3061 | cagccccgag gagccgctgc cttcaccgcc ccgacgcctt ttatcctttt ttaaacgctc |
| 3121 | ttgggtttta tgtccgctgc ttcttggttg ccgagacaga gagatggtgg tctcgggcca |
| 3181 | gcccctcctc tccccgcctt ctgggaggag gaggtcacac gctgatgggc actggagagg |
| 3241 | ccagaagaga ctcagaggag cgggctgcct tccgcctggg gctccctgtg acctctcagt |
| 3301 | cccctggccc ggccagccac cgtccccagc acccaagcat gcaattgcct gtcccccccg |
| 3361 | gccagcctcc ccaacttgat gtttgtgttt tgtttggggg gatatttttc ataattattt |
| 3421 | aaaagacagg ccgggcgcgg tggctcacgt ctgtaatccc agcactttgg gaggctgagg |
| 3481 | cgggcggatc acctgaggtt gggagttcaa gaccagcctg gccaacatgg ggaaaccccg |
| 3541 | tctctactaa aaatacaaaa aattagccgg gtgtggtggc gcgtgcctat aatcccagct |
| 3601 | actcgggagg ctgaggcagg agaatcgctt gaacccggga ggtggaggtt gcggtgagcc |
| 3661 | aagatcgcac cattgcactc cagcctgggc aacaagagcg aaactctgtc tcaaaataaa |
| 3721 | taaaaaataa aagacagaaa gcaaggggtg cctaaatcta gacttggggt ccacaccggg |
| 3781 | cagcggggtt gcaacccagc acctggtagg ctccatttct tcccaagccc gagcagaggg |
| 3841 | tcatgcgggc cccacaggag aagcggccag ggcccgcggg gggcaccacc tgtggacagc |
| 3901 | cctcctgtcc ccaagctttc aggcaggcac tgaaacgcac cgaacttcca cgctctgctg |
| 3961 | gtcagtggcg gctgtcccct ccccagccca gccgcccagc cacatgtgtc tgcctgaccc |
| 4021 | gtacacacca ggggttccgg ggttgggagc tgaaccatcc ccacctcagg gttatatttc |
| 4081 | cctctcccct tccctccccg ccaagagctc tgccaggggc gggcaaaaaa aaaagtaaaa |
| 4141 | agaaaagaaa aaaaaaaaaa agaaacaaac cacctctaca tattatggaa agaaaatatt |
| 4201 | tttgtcgatt cttattcttt tataattatg cgtggaagaa gtagacacat taaacgattc |
| 4261 | cagttggaaa aaaaaaaaaa aaaaaa |

In some embodiments of the methods of the disclosure, the wild type human DPP9 gene of the disclosure consists of or comprises the amino acid sequence (Genbank Accession number: NP 631898.3):

| (SEQ ID NO: 30) | |
|---|---|
| 1 | mrkvkklrld kentgswrsf slnsegaerm attgtptadr gdaaatddpa arfqvqkhsw |
| 61 | dglrsiihgs rkysglivnk aphdfqfvqk tdesgphshr lyylgmpygs rensllysei |
| 121 | pkkvrkeall llswkqmldh fqatphhgvy sreeellrer krlgvfgits ydfhsesglf |
| 181 | lfqasnslfh crdggkngfm vspmkpleik tqcsgprmdp kicpadpaff sfinnsdlwv |
| 241 | anietgeerr ltfchqglsn vlddpksagv atfviqeefd rftgywwcpt aswegseglk |
| 301 | tlrilyeevd esevevihvp spaleerktd syryprtgsk npkialklae fqtdsqgkiv |
| 361 | stqekelvqp fsslfpkvey iaragwtrdg kyawamfldr pqqwlqlvll ppalfipste |
| 421 | neeqrlasar avprnvqpyv vyeevtnvwi nvhdifypfp qsegedelcf lranecktgf |
| 481 | chlykvtavl ksqgydwsep fspgedefkc pikeeialts gewevlarhg skiwvneetk |
| 541 | lvyfqgtkdt plehhlyvvs yeaageivrl ttpgfshscs msqnfdmfvs hyssystppc |
| 601 | vhvyklsgpd ddplhkqprf wasmmeaasc ppdyvppeif hfhtrsdvrl ygmiykphal |
| 661 | qpgkkhptvl fvyggpqvql vnnsfkgiky lrlntlaslg yavvvidgrg scqrglrfeg |
| 721 | alknqmgqve iedqveglqf vaekygfidl srvaihgwsy ggflslmgli hkpqvfkvai |
| 781 | agapvtvwma ydtgyterym dvpennqhgy eagsvalhve klpnepnrll ilhgfldenv |
| 841 | hffhtnflvs qliragkpyq lqiypnerhs ircpesgehy evtllhflqe yl |

In some embodiments of the methods of the disclosure, the wild type human SIGLEC14 gene of the disclosure consists of or comprises the nucleic acid sequence (Genbank Accession number: NM_001098612.1):

| (SEQ ID NO: 31) | |
|---|---|
| 1 | actcaccctc cggcttcctg tcggggcttt ctcagcccca ccccacgttt ggacatttgg |
| 61 | agcatttcct tccctgacag ccggacctgg gactgggctg gggccctggc ggatggagac |
| 121 | atgctgcccc tgctgctgct gcccctgctg tggggggggt ccctgcagga gaagccagtg |
| 181 | tacgagctgc aagtgcagaa gtcggtgacg gtgcaggagg gcctgtgcgt ccttgtgccc |
| 241 | tgctccttct cttacccctg gagatcctgg tattcctctc ccccactcta cgtctactgg |
| 301 | ttccgggacg gggagatccc atactacgct gaggttgtgg ccacaaacaa cccagacaga |
| 361 | agagtgaagc cagagaccca gggccgattc cgcctccttg gggatgtcca gaagaagaac |
| 421 | tgctccctga gcatcggaga tgccagaatg gaggacacgg gaagctattt cttccgcgtg |
| 481 | gagagaggaa gggatgtaaa atatagctac caacagaata agctgaactt ggaggtgaca |
| 541 | gccctgatag agaaacccga catccacttt ctggagcctc tggagtccgg ccgccccaca |
| 601 | aggctgagct gcagccttcc aggatcctgt gaagcgggac cacctctcac attctcctgg |
| 661 | acggggaatg ccctcagccc cctggacccc gagaccaccc gctcctcgga gctcaccctc |
| 721 | acccccaggc ccgaggacca tggcaccaac ctcacctgtc aggtgaaacg ccaaggagct |
| 781 | caggtgacca cggagagaac tgtccagctc aatgtctcct atgctccaca gaacctcgcc |
| 841 | atcagcatct tcttcagaaa tggcacaggc acagccctgc ggatcctgag caatggcatg |
| 901 | tcggtgccca tccaggaggg ccagtccctg ttcctcgcct gcacagttga cagcaacccc |
| 961 | cctgcctcac tgagctggtt ccgggaggga aaagccctca atccttccca gacctcaatg |
| 1021 | tctgggaccc tggagctgcc taacatagga gctagagagg gaggggaatt cacctgccgg |
| 1081 | gttcagcatc cgctgggctc ccagcacctg tccttcatcc tttctgtgca gagaagctcc |
| 1141 | tcttcctgca tatgtgtaac tgagaaacag cagggctcct ggcccctcgt cctcaccctg |
| 1201 | atcagggggg ctctcatggg ggctggcttc ctcctcacct atggcctcac ctggatctac |
| 1261 | tataccaggt gtggaggccc ccagcagagc agggctgaga ggcctggctg agcccctccc |
| 1321 | gctcaagaca gaactgaggt gtggacactt agccctgtgg gacacatgca ggacatcact |
| 1381 | gtcagcttct ttctggaagc tcacatccca ctgactaccc ctcttttcct tcctgcccca |
| 1441 | taccccttct acttattccc ctctgcttgt gagtcttgcc ccaccacacc tgcatcccca |
| 1501 | tctgcacccc atcccctctc cacctgccct tctcttccct ctccatccac catctccagc |
| 1561 | cctgtgaagg gaatgtactt tcggtcttat acccccatta cccattaccc aaaagttacc |
| 1621 | tttttttttt tttttttttt ttgagacaga gtctcactct gttgcacagg ctggagttca |
| 1681 | gtggcacaat ctccgttcac tgcaacctcc acctctgggg ttcaagcaat tctcctgcct |
| 1741 | cagcctccct agtagctggg attacaggtg cctgccacca catccagtta attttttttt |
| 1801 | tttgtatgtt agtagagatg gggttttacc atgttggcca ggtctcgaac tcctgacctc |
| 1861 | aagcaatcca ctgcattggc ctcccaaagt gctggcatta caggtatgag ccaccgtgcc |
| 1921 | tggctgccaa aagttacctt cttaacactt gaatttctgg tctcctcagc ttccctatcc |
| 1981 | atataggcac agagaggcag catttgtttt ccagttaaaa ctctacctca ttgtgattat |
| 2041 | tatccaatac aattgttaca aaataagtaa aacttttatg aaacaataca acataactga |
| 2101 | ttttactctt taa |

In some embodiments of the methods of the disclosure, the wild type human SIGLEC14 gene of the disclosure consists of or comprises the amino acid sequence (Genbank Accession number: NP_001092082.1):

| (SEQ ID NO: 32) | |
|---|---|
| 1 | mlpllllpll wggslqekpv yelqvqksvt vqeglcvlvp csfsypwrsw ysspplyvyw |
| 61 | frdgeipyya evvatnnpdr rvkpetqgrf rllgdvqkkn cslsigdarm edtgsyffrv |
| 121 | ergrdvkysy qqnklnlevt aliekpdihf leplesgrpt rlscslpgsc eagppltfsw |
| 181 | tgnalspldp ettrsseltl tprpedhgtn ltcqvkrqga qvttertvql nvsyapqnla |
| 241 | isiffrngtg talrilsngm svpiqegqsl flactvdsnp paslswfreg kalnpsqtsm |
| 301 | sgtlelpnig areggeftcr vqhplgsqhl sfilsvqrss sscicvtekq qgswplvltl |
| 361 | irgalmgagf lltygltwiy ytrcggpqqs raerpg |

In some embodiments of the methods of the disclosure, the wild type human ADM2 gene of the disclosure consists of or comprises the nucleic acid sequence (Genbank Accession number: NM001253845.1):

| (SEQ ID NO: 33) | |
|---|---|
| 1 | cgcccacgcc cggcgccccg accgcggagg actccccgag ccccgcccgc catggcccgg |
| 61 | atcccgacgg ccgccctggg ttgcatcagc ctcctctgcc tgcagctccc tggctcgctg |
| 121 | tcccgcagcc tgggcgggga cccgcgaccc gtcaaaccca gggagccccc agcccggagc |
| 181 | ccttccagca gcctgcagcc caggcacccc gcaccccgac ctgtggtctg gaagcttcac |
| 241 | cgggccctcc aggcacagag gggtgccggc ctggcccctg ttatgggtca gcctctccgg |
| 301 | gatggtggcc gccaacactc gggcccccga agacactcgg gcccccgcag gacccaagcc |
| 361 | cagctcctgc gagtgggctg tgtgctgggc acctgccagg tgcagaatct cagccaccgc |
| 421 | ctgtggcaac tcatgggacc ggccggccgg caggactcag ctcctgtgga ccccagcagc |
| 481 | ccccacagct atggctgagg tggggccggg ccacacccct gcccatccca gccagggtgc |
| 541 | tgtgcccccg tccagagctg cagctgagcc ccatctgaag cccagtccct cggagctgca |
| 601 | gacagcaggt cctgcagcaa caatacctgc acggctttgc acacgtaaac ctaggctggt |
| 661 | ctacacgcag tgctggtacg tcaaggagcc taaacaccct gaaattgtga ccccctgggg |
| 721 | gacagctgcc agacacagct ggcggcagca ccagatgcta agcgcttcag agaggaggtg |
| 781 | tctgcccaga gatgtggagc agaagctggg ccctgaacac acggggccat gtctggacga |
| 841 | gcaggggaga gaggctgaac tggccagaag tggcccctcc gctgctggtc cagtcagact |
| 901 | gaagcccggc cttgtgcctg ggctgttcct gctctcatgc acaaccagcc cttccacgtg |
| 961 | cctgcctgtg ggacaggagg gggagcgtgg gatgctgtag cccccggggt tgggcaaggg |
| 1021 | aaggatggtg gccctccaga ggtcatgaag ggacctctgt ggctccagct gccaaccctg |
| 1081 | gagcccagac cgaggtggcc atggagactc cacctggatc ccctgtagga ggccagggag |
| 1141 | gggaactcag cagttcagga gccaccccaa accattctgg gacagggaca cccctttcta |
| 1201 | ccccagggca gggcagggct gggtggggca agatccccca gcccgactag acccacctca |
| 1261 | cctgaagggg gtgagaccct tgttggcagc cagacaaggg tggggctcca caggcagcac |
| 1321 | aggcgcccca ccaccaccca gtttggggac ccagtgggac caggtgcggg ggcagagggt |
| 1381 | gacttaccaa gagccaggga gggcagccca ggcccaagtg acagcaagaa caagaaccac |
| 1441 | tgccggcgtg cacagacttg gtgtgtgtcc ttccctgggg ggacggggga ctcacatgtg |
| 1501 | cctgccactg gagcctctca accgtccagc agaacacggg gttcagaaag ggctccttct |
| 1561 | gctatttagc gaacactgag catttaattt acaaatgttt gctagggtca ccctctcggc |
| 1621 | catcccacga gggtcgccat gatcacccca actctagagg ccgcagcaga gctcaggaca |
| 1681 | ttcccccaca gagcttgccc ctcagttcct acctccaagg gggagggtcc tggaagcgcc |
| 1741 | cacccaggcg ccgcccctgt gcttgctccc cgagctcagg gattgccgag tccacgtaac |
| 1801 | tgacctgtac tccacgaggc cctgtgggaa cggtccaggc tggtcctgcc ctgtggaggc |
| 1861 | ctccgtgcac tgagagatgt actaggattg cagcaaaggt ggtcagggtg atgggccgca |
| 1921 | cagcgaggca gtcaaggcca gctccctggg agaagcactg ggtcaggtga ggtctgagga |
| 1981 | cagcaggcct tccctagggg aaggagctgg gagtgccaag gccccaggtg cacaggaggc |
| 2041 | gtggctgctg agaggctgca gggtggaggg gcctcggcct cagagtcatg tgccctgtga |
| 2101 | ccactgaagg gtgtcagcag agcacacggc atgaggacag agggaggggc acggggagtg |
| 2161 | aaggaggggg ccctggggca aggctcgggg gtcaggagct cagcgtccgc tactcagccc |
| 2221 | agccaaaacc ctcccagacg tctcctctcc tgcctgggca aagtccagct tggcaccccg |
| 2281 | tctggggcct gcctgtggtc agggccaagt gttccctcct ccaggaaagc ctttaccctc |
| 2341 | ctcatgccct gtagtcagga ggccgcctgc tgtaaccctc cgtgtcgcct cgggtgcgaa |
| 2401 | atcagaccca cctgacacca tcacgcggag gcccagcagc acctgcaccc acttccagct |
| 2461 | gctctggcca aaatctccgc tcggccaggc cccgtggctc acacctgtaa tcctagcaca |
| 2521 | ttgggaggcc aaggcaggca catcacctga gttcaggagt tcaagaccag cctggccaac |
| 2581 | atggtgaaat cccgtctcta ctaaaaacag aaaattatcc gggcgtggtg gcacatgact |
| 2641 | gtaatcccag ctactcagga ggctgaggca ggaggatcac ttgaacctgg gaggcggagg |
| 2701 | ttgcagtgag ctgagattgc gccattgcac tccagcctgg gcaacaagag caaaattctg |
| 2761 | cctcaaaaaa aaaaatagta ataatacaaa aattagctgg gcgtggtggc acatgccagt |
| 2821 | aattccatct actcgggagg ctgaggcagg agaatcgtct aagcccggga ggtggaggtt |
| 2881 | gcagtgagcc cagatggcgc tgctgcactc aagcttggat gacagagcaa gactccgttt |
| 2941 | caaaaaaaaa aaacctcctc tcttccttca caccttcctc tgaatcccac ccggtcccac |
| 3001 | ctcctgaacc tatccagaca ccttctcctg acccaggcac cacctgcttt cggggcgatg |
| 3061 | gccgtagcct cctcccaggc acctgtctgc atccctctgg ccagtgcatg ctgagcacgt |
| 3121 | gacctacccg tgttgggaca cgtgaggata cagccttgac ccccaggggc tgacattcta |
| 3181 | gggggagata gaaggagaca aacgtagaag gtagaataag tgggtggtgg agtggcaggg |
| 3241 | agtgctgagt gccacaggaa gtcagacaag gaaggagagt gtggggcagg tgccgtttaa |
| 3301 | atggggggcg ctggggtctc ctcacagttg cttctcagct cagctgtgcc aggatcttgt |
| 3361 | tgagtcaggt cagctgccca cagccctctt gcctgacccc tgaagcccag aactctgatc |
| 3421 | ttcacagccc taggtatggc cccagcaccc cactgccctc tctcctgccc cagccgactg |
| 3481 | ctgttcccag acttccctgg ccacgctcca agacgccagc tctgccgcgg gcactttgtt |
| 3541 | ctcacggtgt cctccatgcc tgcagggccc atgcatggga agttgcgttg gcggcctggg |
| 3601 | tgttggcggt tccgtgcctg ctccaactct ccgtgaggcc cctctcccag agcctgacac |
| 3661 | actctgtggc cgaactctag gcaggtgccc ctgagtcctt tcctcgacga ggcctgaccc |
| 3721 | catccccatc ctcgctgggc ccgccgaccc cggtgttagc aagaatcctc taaatcagtt |
| 3781 | tatggagaat tacccaccct cgatatctga tcccattcct catctcccac ccttgatctc |
| 3841 | atcaccctgc cggcctcctg caagatcctc attgagccac tccagtgaga atccccctac |
| 3901 | cctcgaaggc cgccctaaca acttcccatc cgctgacccc tccaacgcca tcaatctcca |
| 3961 | gctgtggttg ttgaactcgg aggtgagctc ctctcaccac tctcttgaat aaagcttttc |
| 4021 | tcaccatttt aaaaaaaaaa aaaaa |

In some embodiments of the methods of the disclosure, the wild type human ADM2 gene of the disclosure consists of or comprises the amino acid sequence (Genbank Accession number: NP_001240774.1):

| (SEQ ID NO: 34) | |
|---|---|
| 1 | mariptaalg cisllclqlp gslsrslggd prpvkprepp arspssslqp rhpaprpvvw |
| 61 | klhralqaqr gaglapvmgq plrdggrqhs gprrhsgprr tqaqllrvgc vlgtcqvqnl |
| 121 | shrlwqlmgp agrqdsapvd pssphsyg |

In some embodiments of the methods of the disclosure, the wild type human TSPAN5 gene of the disclosure consists of or comprises the nucleic acid sequence (Genbank Accession number: NM005723.3):

| (SEQ ID NO: 35) | |
|---|---|
| 1 | aggcgggcgg agcgaggggt gggagggcgc gcgcgaacgg gcgggcgagc aagcgagcgg |
| 61 | cgtctccacc agcatctgcc gcggccgcct ttgcccgaag cccggggacg aaccgacgga |
| 121 | ccgaccgcct ggcgcacgga cgcgggcgct cgctttgtgt tcggggctag cgtcggcgag |
| 181 | gcttgagctt gcagcgcgcg gcttccctgc tttctcgcgg ccaccccggc tccggcggcc |
| 241 | tcggcgcgcg aggggctgga ggtgcgggag ccgctctccg ccggtcggtc cccgcgcggc |
| 301 | tgagcccagg ccgccagcgc cgcggccccg tgcggtgtcc ctgagctcct gctccccgcc |
| 361 | gggctgctcc gagcaacggt gcttcggagc tccaaactcg ggctgccggg gcaagtgtct |
| 421 | tcatgaaccc agaggatgtc cgggaagcac tacaagggtc ctgaagtcag ttgttgcatc |
| 481 | aaatacttca tatttggctt caatgtcata ttttggtttt tgggaataac atttcttgga |
| 541 | attggactgt gggcatggaa tgaaaaagga gttctgtcca acatctcttc catcaccgat |
| 601 | ctcggcggct ttgacccagt ttggctcttc cttgtggtgg gaggagtgat gttcattttg |
| 661 | ggatttgcag ggtgcattgg agcgctacgg gaaaacactt tccttctcaa gtttttttct |
| 721 | gtgttcctgg gaattatttt cttcctggag ctcactgccg gagttctagc atttgttttc |
| 781 | aaagactgga tcaaagacca gctgtatttc tttataaaca acaacatcag agcatatcgg |
| 841 | gatgacattg atttgcaaaa cctcatagac ttcacccagg aatattggca gtgctgtggg |
| 901 | gcttttggag ctgatgattg gaacctaaat atttacttca attgcacaga ttccaatgca |
| 961 | agtcgagagc gatgtggcgt tccattctcc tgctgcacta aagatcccgc agaagatgtc |
| 1021 | atcaacactc agtgtggcta tgatgccagg caaaaaccag aagttgacca gcagattgta |
| 1081 | atctacacga aaggctgtgt gccccagttt gagaagtggt tgcaggacaa tttaaccatc |
| 1141 | gttgctggta ttttcatagg cattgcattg ctgcagatat ttgggatatg cctggcccag |
| 1201 | aatttggtta gcgatatcga agctgtcagg gcgagctggt agaccccctg caaccgctgc |
| 1261 | tgcaagacac tggacagacc cagctttcgg gaccctcccg cgtgccgaac tgatcttcga |
| 1321 | gctgcatgga cctaatcaca gatgcagcct gcagtctcgc ctaatggagc tgccattagg |
| 1381 | ggagtgtaaa actgggaaat gctgctcact gacagaatta aaaaaaaaaa taaccagtat |
| 1441 | gaaagtcgtt gcgccgtgaa tctctactgt agccatgaat ttatggacag ttagatgctt |
| 1501 | accaaaaaag aaaaaaaggg agggtagggg acccagatgt acttgaatgt gcagaaaata |
| 1561 | cattcttgtc ctcatcttcc gtaattggag ggctgggaga ggcagctttg ctcttcacca |
| 1621 | caccttggac ggaccacctt ctttctgttc catggcctga aggagtgcat ctcctcaaag |
| 1681 | actcagcccc tcacctggga gggcagtggt ttgtgggcat ccctccatgt acattttagg |
| 1741 | aaacacttgc aactctcatc tgaagaagaa aacaactcat ctttgggttc agattttgtg |
| 1801 | atggtattca gcaagtcact tgggcgagca cacttggtct atcctggaaa gtctccttat |
| 1861 | aagagaagtt gtgtatttca tgtgcaccga gcaagggcat tggaagacgt catgaggctg |
| 1921 | tattttagca ggactgatcg tttttctaag tagacctgag ctttgtttat cagtgaaatt |
| 1981 | caaggagaaa atgaggttaa tgaagaggta tcagttaaat atccccttct tctcaccctg |
| 2041 | ccaaaattag cagttggatt tttggaaact ctggaatatt ctgggtcatt ttgttttgta |
| 2101 | tgtttgttgt ttttcgtctt ccaaaggtga aagctatgat acagttccac ttaaatttta |
| 2161 | gtgttttctt actcagctca agcattaatt tttgattaag tcttaatctg catgacctgt |
| 2221 | gaatctgaat ccatcatctc cctttcctgc cagcttttct acaaacattg aaatatgtta |
| 2281 | tttggtcagc acttatttcc taggttcaca gccttgggag gttgtggcat gtcctcccag |
| 2341 | tctggctggg aagagaccag ctgtaccatc caaatgcttc cctggtcttg atgatctctt |
| 2401 | ccagagtcga tctgagtggc cttttctgca ccctcccctt ctttctcttt gaatggaatt |
| 2461 | aaacccaatt tggaaacaac attgacccag tcaaaagctt ctaatggttt ctttttcttc |
| 2521 | ctccagtttt agtttgcttt tattaaaaaa agaaaatagt gcatggccat agctccttca |
| 2581 | gttctcttat tgcagactaa ccatcaggat ggtatcaaag cacaaatact ttggagggga |
| 2641 | atgcgttgaa ctggggcaag tactctgtaa cacaaagtgg gaaaccactt cctggtgctg |
| 2701 | ccgctcctgc ccccacttta ggtgggaggg acgagttttg ccctctagat tttaatccag |
| 2761 | ctggtgtcca ccggatgttg ccctcctggg gagcagatat cagtctgtgg aactctggga |
| 2821 | aaaccacagg cacatttttc ggtgcggaca gatttgccag cacataactg ggcagccagc |
| 2881 | tagaatactt tgtggaaatt aagcgaggtt ttccatttca gccccatggt gcatggtggt |
| 2941 | ggccgatgaa tgtgtcagtc tgctcagaga aaggacaaaa aggaaattat tttcaaaact |
| 3001 | gtgttcactg tttgggtgtg tgtatggctc tgcatgtgtg tgtttttgtc tctgtatagg |
| 3061 | tagaggtatt cacatcttac tccgactgta aggttgtctt acttcatctc tgcccccacc |
| 3121 | acagttgcca ttttgtaatg tccttccaac atggagaaga cacgagctct ctccagttgg |
| 3181 | catcatttgt cttttttgtt gattgcctca ttctccagtg aactccatct ggccaattga |
| 3241 | ttcagaatca ggcaagatcc ctgccctttg gcacatccac tgaaaggcca aacagcaagt |
| 3301 | ccgagtgagt tttaaatatt aattaatcac cctttatttt ttacacttga gagtgattgt |
| 3361 | aataaaggct gtcattaata aacttggttc taccttaaaa aaaaaa |

In some embodiments of the methods of the disclosure, the wild type human TSPAN5 gene of the disclosure consists of or comprises the amino acid sequence (Genbank Accession number: NP_005714.2):

| (SEQ ID NO: 52) | |
|---|---|
| 1 | msgkhykgpe vsccikyfif gfnvifwflg itflgiglwa wnekgvlsni ssitdlggfd |
| 61 | pvwlflvvgg vmfilgfagc igalrentfl lkffsvflgi iffleltagv lafvfkdwik |
| 121 | dqlyffinnn irayrddidl qnlidftqey wqccgafgad dwnlniyfnc tdsnasrerc |
| 181 | gvpfscctkd paedvintqc gydarqkpev dqqiviytkg cvpqfekwlq dnltivagif |
| 241 | igiallqifg iclaqnlvsd ieavrasw |

In some embodiments of the methods of the disclosure, the wild type human CAMKK1 gene of the disclosure consists of or comprises the nucleic acid sequence (Genbank Accession number: NM_032294.2, transcript variant 1):

| (SEQ ID NO: 53) | |
|---|---|
| 1 | ctgggcccca gcgaggcggt ggggcggggc ggggcggggc ggggcgcgca gcaggagcga |
| 61 | gtggggccgc ccgccgggcc gcggacactg tcgcccggcg cccaggttcc caacaaggct |
| 121 | acgcagaaga acccccttga ctgaagcaat ggaggggggt ccagctgtct gctgccagga |
| 181 | tcctcgggca gagctggtag aacgggtggc agccatcgat gtgactcact tggaggaggc |
| 241 | agatggtggc ccagagccta ctagaaacgg tgtggacccc ccaccacggg ccagagctgc |
| 301 | ctctgtgatc cctggcagta cttcaagact gctcccagcc cggcctagcc tctcagccag |
| 361 | gaagctttcc ctacaggagc ggccagcagg aagctatctg gaggcgcagg ctgggcctta |
| 421 | tgccacgggg cctgccagcc acatctcccc ccgggcctgg cggaggccca ccatcgagtc |
| 481 | ccaccacgtg gccatctcag atgcagagga ctgcgtgcag ctgaaccagt acaagctgca |
| 541 | gagtgagatt ggcaagggtg cctacggtgt ggtgaggctg gcctacaacg aaagtgaaga |
| 601 | cagacactat gcaatgaaag tcctttccaa aaagaagtta ctgaagcagt atggctttcc |
| 661 | acgtcgccct cccccgagag ggtcccaggc tgcccaggga ggaccagcca agcagctgct |
| 721 | gcccctggag cgggtgtacc aggagattgc catcctgaag aagctggacc acgtgaatgt |
| 781 | ggtcaaactg atcgaggtcc tggatgaccc agctgaggac aacctctatt tggtgtttga |
| 841 | cctcctgaga aaggggcccg tcatggaagt gccctgtgac aagcccttct cggaggagca |
| 901 | agctcgcctc tacctgcggg acgtcatcct gggcctcgag tacttgcact gccagaagat |
| 961 | cgtccacagg gacatcaagc catccaacct gctcctgggg gatgatgggc acgtgaagat |
| 1021 | cgccgacttt ggcgtcagca accagtttga ggggaacgac gctcagctgt ccagcacggc |
| 1081 | gggaacccca gcattcatgg cccccgaggc catttctgat tccggccaga gcttcagtgg |
| 1141 | gaaggccttg gatgtatggg ccactggcgt cacgttgtac tgctttgtct atgggaagtg |
| 1201 | cccattcatc gacgatttca tcctggccct ccacaggaag atcaagaatg agcccgtggt |
| 1261 | gtttcctgag gagccagaaa tcagcgagga gctcaaggac ctgatcctga agatgttaga |
| 1321 | caagaatccc gagacgagaa ttggggtgcc agacatcaag ttgcaccctt gggtgaccaa |
| 1381 | gaacggggag gagccccttc cttcggagga ggagcactgc agcgtggtgg aggtgacaga |
| 1441 | ggaggaggtt aagaactcag tcaggctcat ccccagctgg accacggtga tcctggtgaa |
| 1501 | gtccatgctg aggaagcgtt cctttgggaa cccgtttgag ccccaagcac ggagggaaga |
| 1561 | gcgatccatg tctgctccag gaaacctact ggtgaaagaa gggtttggtg aagggggcaa |
| 1621 | gagcccagag ctccccggcg tccaggaaga cgaggctgca tcctgagccc ctgcatgcac |
| 1681 | ccagggccac ccggcagcac actcatcccg cgcctccaga ggcccacccc tcatgcaaca |
| 1741 | gccgcccccg caggcagggg gctggggact gcagccccac tcccgcccct cccccatcgt |
| 1801 | gctgcatgac ctccacgcac gcacgtccag ggacagactg gaatgtatgt catttggggt |
| 1861 | cttgggggca gggctcccac gaggccatcc tcctcttctt ggacctcctt ggcctgaccc |
| 1921 | attctgtggg gaaaccgggt gcccatggag cctcagaaat gccacccggc tggttggcat |
| 1981 | ggcctggggc aggaggcaga ggcaggagac caagatggca ggtggaggcc aggcttacca |
| 2041 | caacggaaga gacctcccgc tggggccggg caggcctggc tcagctgcca caggcatatg |
| 2101 | gtggagaggg gggtaccctg cccaccttgg ggtggtggca ccagagctct tgtctattca |
| 2161 | gacgctggta tgggggctcg gacccctcac tggggacagg gccagtgttg gagaattctg |
| 2221 | attccttttt tgttgtcttt tacttttgtt tttaacctgg gggttcgggg agaggccctg |
| 2281 | cttgggaaca tctcacgagc tttcctacat cttccgtggt tcccagcaca gcccaagatt |
| 2341 | atttggcagc caagtggatg gaactaactt tcctggactg tgtttcgcat tcggcgttat |
| 2401 | ctggaaagtg gactgaacgg aatcaagctc tgagcagagg cctgaagcgg aagcaccaca |
| 2461 | tcgtccctgc ccatctcact ctctcccttg atgatgcccc tagagctgag gctggagaag |
| 2521 | acaccagggc tgactttgac cgagggccat ggacgcgaca ggcctgtggc cctgcgcatg |
| 2581 | ctgaaataac tggaacccag cctctcctcc tacaccggcc tacccatctg ggcccaagag |
| 2641 | ctgcactcac actcctacaa cgaaggacaa actgtccagg tcggagggat cacgagacac |
| 2701 | agaacctgga ggggtgtgca cgctggcagg tggcctctgc ggcaattgcc tcaccctgag |
| 2761 | gacatcagca gtcagcctgc tcagagcggg ggtgctggag cgcgtgcaga cacagctctt |
| 2821 | ccggagcagc cttcaccttc tctctgggat cagtgtccgg ctggccgacg tggcatttgc |
| 2881 | tgaccgaatg ctcatagagg ttgaccccca cagggtcacg caggactcgg acactgccct |
| 2941 | ggaaacatgg atggacaagg gcttttggcc acaggtgtgg gtgtcctgtt ggaggagggc |
| 3001 | ttgtttggag aagggaggct ggctggggga gaaacccgga tcccgctgca tctccgcgcc |
| 3061 | tgtgggtgca tgtcgcgtgc tcatctgttg cacacagctc actcgtatgt cctgcactgg |
| 3121 | tacatgcatc tgtaatacag tttctacgtc tatttaaggc taggagccga atgtgcccca |
| 3181 | ttgtcagtgg gtccacgttt ctccccggct cctctgggct aaggcagtgt ggcccgaagc |
| 3241 | ttaaaaagtt actcggtact gtttttaaga acacttttat agagttagtg gaaggcaagt |
| 3301 | taagagccaa tcactgatcc ccaagtgttt cttgagcatc tggtctgggg ggaccacttt |
| 3361 | gatcggaccc acccttggaa agctcagggg taggcccagg tgggatgctc accctgtcac |
| 3421 | tgagggtttt ggttggcatc gttgtttttg aatgtagcac aagcgatgag caaactctat |
| 3481 | aagagtgttt taaaaattaa cttcccagga agtgagttaa aaacaataaa agccctttct |
| 3541 | tgagttaaaa agaaaaaaaa aaaaaaaaaa aaaaaaaaaa aaa |

In some embodiments of the methods of the disclosure, the wild type human CAMKK1 gene of the disclosure consists of or comprises the amino acid sequence (Genbank Accession number: NP_115670.1, transcript variant 1):

| (SEQ ID NO: 54) | |
|---|---|
| 1 | meggpavccq dpraelverv aaidvthlee adggpeptrn gvdppprara asvipgstsr |
| 61 | llparpslsa rklslqerpa gsyleaqagp yatgpashis prawrrptie shhvaisdae |
| 121 | dcvqlnqykl qseigkgayg vvrlaynese drhyamkvls kkkllkqygf prrppprgsq |
| 181 | aaqggpakql lplervyqei ailkkldhvn vvklievldd paednlylvf dllrkgpvme |
| 241 | vpcdkpfsee qarlylrdvi lgleylhcqk ivhrdikpsn lllgddghvk iadfgvsnqf |
| 301 | egndaqlsst agtpafmape aisdsgqsfs gkaldvwatg vtlycfvygk cpfiddfila |
| 361 | lhrkiknepv vfpeepeise elkdlilkml dknpetrigv pdiklhpwvt kngeeplpse |
| 421 | eehcsvvevt eeevknsvrl ipswttvilv ksmlrkrsfg npfepqarre ersmsapgnl |
| 481 | lvkegfgegg kspelpgvqe deaas |

In some embodiments of the methods of the disclosure, the wild type human CAMKK1 gene of the disclosure consists of or comprises the nucleic acid sequence (Genbank Accession number: NM_172206.1, transcript variant 2):

| (SEQ ID NO: 55) | |
|---|---|
| 1 | agcagaacag agtatgcaat ttgggaagct gtggtgtggc tgcagtggag agttcccaac |
| 61 | aaggctacgc agaagaaccc ccttgactga agcaatggag gggggtccag ctgtctgctg |
| 121 | ccaggatcct cgggcagagc tggtagaacg ggtggcagcc atcgatgtga ctcacttgga |
| 181 | ggaggcagat ggtggcccag agcctactag aaacggtgtg gaccccccac cacgggccag |
| 241 | agctgcctct gtgatccctg gcagtacttc aagactgctc ccagcccggc ctagcctctc |
| 301 | agccaggaag ctttccctac aggagcggcc agcaggaagc tatctggagg cgcaggctgg |
| 361 | gccttatgcc acggggcctg ccagccacat ctccccccgg gcctggcgga ggcccaccat |
| 421 | cgagtcccac cacgtggcca tctcagatgc agaggactgc gtgcagctga accagtacaa |
| 481 | gctgcagagt gagattggca agggtgccta cggtgtggtg aggctggcct acaacgaaag |
| 541 | tgaagacaga cactatgcaa tgaaagtcct ttccaaaaag aagttactga agcagtatgg |
| 601 | ctttccacgt cgccctcccc cgagagggtc ccaggctgcc cagggaggac cagccaagca |
| 661 | gctgctgccc ctggagcggg tgtaccagga gattgccatc ctgaagaagc tggaccacgt |
| 721 | gaatgtggtc aaactgatcg aggtcctgga tgacccagct gaggacaacc tctatttggt |
| 781 | gtttgacctc ctgagaaagg ggcccgtcat ggaagtgccc tgtgacaagc ccttctcgga |
| 841 | ggagcaagct cgcctctacc tgcgggacgt catcctgggc ctcgagtact tgcactgcca |
| 901 | gaagatcgtc cacagggaca tcaagccatc caacctgctc ctgggggatg atgggcacgt |
| 961 | gaagatcgcc gactttggcg tcagcaacca gtttgagggg aacgacgctc agctgtccag |
| 1021 | cacggcggga accccagcat tcatggcccc cgaggccatt tctgattccg gccagagctt |
| 1081 | cagtgggaag gccttggatg tatgggccac tggcgtcacg ttgtactgct ttgtctatgg |
| 1141 | gaagtgccca ttcatcgacg atttcatcct ggccctccac aggaagatca agaatgagcc |
| 1201 | cgtggtgttt cctgaggagc cagaaatcag cgaggagctc aaggacctga tcctgaagat |
| 1261 | gttagacaag aatcccgaga cgagaattgg ggtgccagac atcaagttgc acccttgggt |
| 1321 | gaccaagaac ggggaggagc cccttccttc ggaggaggag cactgcagcg tggtggaggt |
| 1381 | gacagaggag gaggttaaga actcagtcag gctcatcccc agctggacca cggtgatcct |
| 1441 | ggtgaagtcc atgctgagga agcgttcctt tgggaacccg tttgagcccc aagcacggag |
| 1501 | ggaagagcga tccatgtctg ctccaggaaa cctactggtg aaagaagggt ttggtgaagg |
| 1561 | gggcaagagc ccagagctcc ccggcgtcca ggaagacgag gctgcatcct gagcccctgc |
| 1621 | atgcacccag ggccacccgg cagcacactc atcccgcgcc tccagaggcc cacccctcat |
| 1681 | gcaacagccg cccccgcagg cagggggctg gggactgcag ccccactccc gcccctcccc |
| 1741 | catcgtgctg catgacctcc acgcacgcac gtccagggac agactggaat gtatgtcatt |
| 1801 | tggggtcttg ggggcagggc tcccacgagg ccatcctcct cttcttggac ctccttggcc |
| 1861 | tgacccattc tgtggggaaa ccgggtgccc atggagcctc agaaatgcca cccggctggt |
| 1921 | tggcatggcc tggggcagga ggcagaggca ggagaccaag atggcaggtg gaggccaggc |
| 1981 | ttaccacaac ggaagagacc tcccgctggg gccgggcagg cctggctcag ctgccacagg |
| 2041 | catatggtgg agaggggggt accctgccca ccttggggtg gtggcaccag agctcttgtc |
| 2101 | tattcagacg ctggtatggg ggctcggacc cctcactggg gacagggcca gtgttggaga |
| 2161 | attctgattc cttttttgtt gtcttttact tttgttttta acctgggggt tcggggagag |
| 2221 | gccctgcttg ggaacatctc acgagctttc ctacatcttc cgtggttccc agcacagccc |
| 2281 | aagattattt ggcagccaag tggatggaac taactttcct ggactgtgtt tcgcattcgg |
| 2341 | cgttatctgg aaagtggact gaacggaatc aagctctgag cagaggcctg aagcggaagc |
| 2401 | accacatcgt ccctgcccat ctcactctct cccttgatga tgcccctaga gctgaggctg |
| 2461 | gagaagacac cagggctgac tttgaccgag ggccatggac gcgacaggcc tgtggccctg |
| 2521 | cgcatgctga aataactgga acccagcctc tcctcctaca ccggcctacc catctgggcc |
| 2581 | caagagctgc actcacactc ctacaacgaa ggacaaactg tccaggtcgg agggatcacg |
| 2641 | agacacagaa cctggagggg tgtgcacgct ggcaggtggc ctctgcggca attgcctcac |
| 2701 | cctgaggaca tcagcagtca gcctgctcag agcgggggtg ctggagcgcg tgcagacaca |
| 2761 | gctcttccgg agcagccttc accttctctc tgggatcagt gtccggctgg ccgacgtggc |
| 2821 | atttgctgac cgaatgctca tagaggttga cccccacagg gtcacgcagg actcggacac |
| 2881 | tgccctggaa acatggatgg acaagggctt ttggccacag gtgtgggtgt cctgttggag |
| 2941 | gagggcttgt ttggagaagg gaggctggct gggggagaaa cccggatccc gctgcatctc |
| 3001 | cgcgcctgtg ggtgcatgtc gcgtgctcat ctgttgcaca cagctcactc gtatgtcctg |
| 3061 | cactggtaca tgcatctgta atacagtttc tacgtctatt taaggctagg agccgaatgt |
| 3121 | gccccattgt cagtgggtcc acgtttctcc ccggctcctc tgggctaagg cagtgtggcc |
| 3181 | cgaagcttaa aaagttactc ggtactgttt ttaagaacac ttttatagag ttagtggaag |
| 3241 | gcaagttaag agccaatcac tgatccccaa gtgtttcttg agcatctggt ctggggggac |
| 3301 | cactttgatc ggacccaccc ttggaaagct caggggtagg cccaggtggg atgctcaccc |
| 3361 | tgtcactgag ggttttggtt ggcatcgttg tttttgaatg tagcacaagc gatgagcaaa |
| 3421 | ctctataaga gtgttttaaa aattaacttc ccaggaagtg agttaaaaac aataaaagcc |
| 3481 | ctttcttgag ttaaaaagaa aaaaaaaaaa aaaaaaaaaa aaaaaaaaa |

In some embodiments of the methods of the disclosure, the wild type human CAMKK1 gene of the disclosure consists of or comprises the amino acid sequence (Genbank Accession number: NP_757343.2, transcript variant 2):

| (SEQ ID NO: 56) | |
|---|---|
| 1 | mqfgklwcgc sgefptrlrr rtplteameg gpavccqdpr aelvervaai dvthleeadg |
| 61 | gpeptrngvd pppraraasv ipgstsrllp arpslsarkl slgerpagsy leaqagpyat |
| 121 | gpashispra wrrptieshh vaisdaedcv qlnqyklqse igkgaygvvr laynesedrh |
| 181 | yamkvlskkk llkqygfprr ppprgsqaaq ggpakqllpl ervyqeiail kkldhvnvvk |
| 241 | lievlddpae dnlylvfdll rkgpvmevpc dkpfseeqar lylrdvilgl eylhcqkivh |
| 301 | rdikpsnlll gddghvkiad fgvsnqfegn daqlsstagt pafmapeais dsgqsfsgka |
| 361 | ldvwatgvtl ycfvygkcpf iddfilalhr kiknepvvfp eepeiseelk dlilkmldkn |
| 421 | petrigvpdi klhpwvtkng eeplpseeeh csvvevteee vknsvrlips wttvilvksm |
| 481 | lrkrsfgnpf epqarreers msapgnllvk egfgeggksp elpgvqedea as |

In some embodiments of the methods of the disclosure, the wild type human CAMKK1 gene of the disclosure consists of or comprises the nucleic acid sequence (Genbank Accession number: NM_172207.2, transcript variant 3):

| (SEQ ID NO: 57) | |
|---|---|
| 1 | ctgggcccca gcgaggcggt ggggcggggc ggggcggggc ggggcgcgca gcaggagcga |
| 61 | gtggggccgc ccgccgggcc gcggacactg tcgcccggcg cccaggttcc caacaaggct |
| 121 | acgcagaaga acccccttga ctgaagcaat ggaggggggt ccagctgtct gctgccagga |
| 181 | tcctcgggca gagctggtag aacgggtggc agccatcgat gtgactcact tggaggaggc |
| 241 | agatggtggc ccagagccta ctagaaacgg tgtggacccc ccaccacggg ccagagctgc |
| 301 | ctctgtgatc cctggcagta cttcaagact gctcccagcc cggcctagcc tctcagccag |
| 361 | gaagctttcc ctacaggagc ggccagcagg aagctatctg gaggcgcagg ctgggcctta |
| 421 | tgccacgggg cctgccagcc acatctcccc ccgggcctgg cggaggccca ccatcgagtc |
| 481 | ccaccacgtg gccatctcag atgcagagga ctgcgtgcag ctgaaccagt acaagctgca |
| 541 | gagtgagatt ggcaagggtg cctacggtgt ggtgaggctg gcctacaacg aaagtgaaga |
| 601 | cagacactat gcaatgaaag tcctttccaa aaagaagtta ctgaagcagt atggctttcc |
| 661 | acgtcgccct cccccgagag ggtcccaggc tgcccaggga ggaccagcca agcagctgct |
| 721 | gcccctggag cgggtgtacc aggagattgc catcctgaag aagctggacc acgtgaatgt |
| 781 | ggtcaaactg atcgaggtcc tggatgaccc agctgaggac aacctctatt tggccctgca |
| 841 | gaaccaggcc cagaatatcc agttagattc aacaaatatc gccaagcccc actccctgct |
| 901 | tccctctgag cagcaagaca gtggatccac gtgggctgcg cgctcagtgt ttgacctcct |
| 961 | gagaaagggg cccgtcatgg aagtgccctg tgacaagccc ttctcggagg agcaagctcg |
| 1021 | cctctacctg cgggacgtca tcctgggcct cgagtacttg cactgccaga agatcgtcca |
| 1081 | cagggacatc aagccatcca acctgctcct gggggatgat gggcacgtga agatcgccga |
| 1141 | ctttggcgtc agcaaccagt ttgaggggaa cgacgctcag ctgtccagca cggcgggaac |
| 1201 | cccagcattc atggcccccg aggccatttc tgattccggc cagagcttca gtgggaaggc |
| 1261 | cttggatgta tgggccactg gcgtcacgtt gtactgcttt gtctatggga agtgcccatt |
| 1321 | catcgacgat ttcatcctgg ccctccacag gaagatcaag aatgagcccg tggtgtttcc |
| 1381 | tgaggagcca gaaatcagcg aggagctcaa ggacctgatc ctgaagatgt tagacaagaa |
| 1441 | tcccgagacg agaattgggg tgccagacat caagttgcac ccttgggtga ccaagaacgg |
| 1501 | ggaggagccc cttccttcgg aggaggagca ctgcagcgtg gtggaggtga cagaggagga |
| 1561 | ggttaagaac tcagtcaggc tcatccccag ctggaccacg gtgatcctgg tgaagtccat |
| 1621 | gctgaggaag cgttcctttg ggaacccgtt tgagccccaa gcacggaggg aagagcgatc |
| 1681 | catgtctgct ccaggaaacc tactggtgta agtactggtg ggccagggac tgccgggcac |
| 1741 | tccctggagt tgggtgggga ggtctgaggc ccatcctccc actctcactg tcgttgggcc |
| 1801 | aaggccagag cctggggact tggccaggtc tcggtgttgg ccccatttgc atctctgtcc |
| 1861 | ccaaggttag tcggggctag aagggacctt ttgggcccag ctcttgcttc attcctgggg |
| 1921 | ccagcatccc tcacacacac acttccaggg atgaggagct cacgcagccc ctccatggga |
| 1981 | caggaagacc cttcttccat gcagcttgat gtcactctct cactgggtcc agcccctctg |
| 2041 | gggcttcaaa tctgtggccc cctcagccct tggcagcctg gcagaggttt gcagacaggc |
| 2101 | tgatgttggc ttcctgtagg aggctggcgg gctgtagagg aggggtgctg gcccctctgc |
| 2161 | ctggccctgg ggactgttgg ctgctctccc aagtggccca ggctgcctgc agccattgct |
| 2221 | ggggctctgt gcccagtcag cactttgtga gtgcttgttc agtgagtaag cagggacagg |
| 2281 | ctggccggtg gaccacggga gaggaacccg cattggccga gggctcccta tggtgagcca |
| 2341 | cgcctgtggg ttcaccacct cctaggaggg tccagaaaag cagctcccca agcctgtgcg |
| 2401 | cctcgtcctc agcagatcca ccttcttcac tataataaaa gccagtctgg gatgctaaaa |
| 2461 | aaaaaaaaaa aaaaaaaaaa aaaaaaaaaa aaaaaaaaaa aaaaaaaaaa aaaaaaaaaa |
| 2521 | aaaaaaaaaa aaaaa |

In some embodiments of the methods of the disclosure, the wild type human CAMKK1 gene of the disclosure consists of or comprises the amino acid sequence (Genbank Accession number: NP_757344.2, transcript variant 3):

| (SEQ ID NO: 58) | |
|---|---|
| 1 | meggpavccq dpraelverv aaidvthlee adggpeptrn gvdppprara asvipgstsr |
| 61 | llparpslsa rklslqerpa gsyleaqagp yatgpashis prawrrptie shhvaisdae |
| 121 | dcvqlnqykl qseigkgayg vvrlaynese drhyamkvls kkkllkqygf prrppprgsq |
| 181 | aaqggpakql lplervyqei ailkkldhvn vvklievldd paednlylal qnqaqniqld |
| 241 | stniakphsl lpseqqdsgs twaarsvfdl lrkgpvmevp cdkpfseeqa rlylrdvilg |
| 301 | leylhcqkiv hrdikpsnll lgddghvkia dfgvsnqfeg ndaqlsstag tpafmapeai |
| 361 | sdsgqsfsgk aldvwatgvt lycfvygkcp fiddfilalh rkiknepvvf peepeiseel |
| 421 | kdlilkmldk npetrigvpd iklhpwvtkn geeplpseee hcsvvevtee evknsvrlip |
| 481 | swttvilvks mlrkrsfgnp fepqarreer smsapgnllv |

In some embodiments of the methods of the disclosure, the wild type human MMP7 gene of the disclosure consists of or comprises the nucleic acid sequence (Genbank Accession number: NM_002423.4):

| (SEQ ID NO: 59) | |
|---|---|
| 1 | gaaaacacca aatcaaccat aggtccaaga acaattgtct ctggacggca gctatgcgac |
| 61 | tcaccgtgct gtgtgctgtg tgcctgctgc ctggcagcct ggccctgccg ctgcctcagg |
| 121 | aggcgggagg catgagtgag ctacagtggg aacaggctca ggactatctc aagagatttt |
| 181 | atctctatga ctcagaaaca aaaaatgcca acagtttaga agccaaactc aaggagatgc |
| 241 | aaaaattctt tggcctacct ataactggaa tgttaaactc ccgcgtcata gaaataatgc |
| 301 | agaagcccag atgtggagtg ccagatgttg cagaatactc actatttcca aatagcccaa |
| 361 | aatggacttc caaagtggtc acctacagga tcgtatcata tactcgagac ttaccgcata |
| 421 | ttacagtgga tcgattagtg tcaaaggctt taaacatgtg gggcaaagag atccccctgc |
| 481 | atttcaggaa agttgtatgg ggaactgctg acatcatgat tggctttgcg cgaggagctc |
| 541 | atggggactc ctacccattt gatgggccag gaaacacgct ggctcatgcc tttgcgcctg |
| 601 | ggacaggtct cggaggagat gctcacttcg atgaggatga acgctggacg gatggtagca |
| 661 | gtctagggat taacttcctg tatgctgcaa ctcatgaact tggccattct ttgggtatgg |
| 721 | gacattcctc tgatcctaat gcagtgatgt atccaaccta tggaaatgga gatccccaaa |
| 781 | attttaaact ttcccaggat gatattaaag gcattcagaa actatatgga aagagaagta |
| 841 | attcaagaaa gaaatagaaa cttcaggcag aacatccatt cattcattca ttggattgta |
| 901 | tatcattgtt gcacaatcag aattgataag cactgttcct ccactccatt tagcaattat |
| 961 | gtcacccttt tttattgcag ttggtttttg aatgtctttc actcctttta aggataaact |
| 1021 | cctttatggt gtgactgtgt cttattcatc tatacttgca gtgggtagat gtcaataaat |
| 1081 | gttacataca caaataaata aaatgtttat tccatggtaa atttaaaaaa aaaaaaaaaa |
| 1141 | aaaaaaaaaa aaa |

In some embodiments of the methods of the disclosure, the wild type human MMP7 gene of the disclosure consists of or comprises the amino acid sequence (Genbank Accession number: NP_002414.1):

| (SEQ ID NO: 60) | |
|---|---|
| 1 | mrltvlcavc llpgslalpl pqeaggmsel qweqaqdylk rfylydsetk nansleaklk |
| 61 | emqkffglpi tgmlnsrvie imqkprcgvp dvaeyslfpn spkwtskvvt yrivsytrdl |
| 121 | phitvdrlvs kalnmwgkei plhfrkvvwg tadimigfar gahgdsypfd gpgntlahaf |
| 181 | apgtglggda hfdederwtd gsslginfly aathelghsl gmghssdpna vmyptygngd |
| 241 | pqnfklsqdd ikgiqklygk rsnsrkk |

In some embodiments of the methods of the disclosure, the wild type human TERC gene of the disclosure consists of or comprises the nucleic acid sequence (Genbank Accession number: NR_001566.1):

| (SEQ ID NO: 61) | |
|---|---|
| 1 | gggttgcgga gggtgggcct gggaggggtg gtggccattt tttgtctaac cctaactgag |
| 61 | aagggcgtag gcgccgtgct tttgctcccc gcgcgctgtt tttctcgctg actttcagcg |
| 121 | ggcggaaaag cctcggcctg ccgccttcca ccgttcattc tagagcaaac aaaaaatgtc |
| 181 | agctgctggc ccgttcgccc ctcccgggga cctgcggcgg gtcgcctgcc cagcccccga |
| 241 | accccgcctg gaggccgcgg tcggcccggg gcttctccgg aggcacccac tgccaccgcg |
| 301 | aagagttggg ctctgtcagc cgcgggtctc tcgggggcga gggcgaggtt caggcctttc |
| 361 | aggccgcagg aagaggaacg gagcgagtcc ccgcgcgcgg cgcgattccc tgagctgtgg |
| 421 | gacgtgcacc caggactcgg ctcacacatg c |

### Definitions

The following definitions are included for the purpose of understanding the present subject matter and for constructing the appended patent claims. Abbreviations used herein have their conventional meaning within the chemical and biological arts.

Unless defined otherwise, technical and scientific terms used herein have the same meaning as commonly understood by a person of ordinary skill in the art. Any methods, devices and materials similar or equivalent to those described herein can be used in the practice of this disclosure. The following definitions are provided to facilitate understanding of certain terms used frequently herein and are not meant to limit the scope of the present disclosure.

As used herein, the term “FILD” refers to fibrotic interstitial lung disease.

As used herein, the term “FIP” refers to Familial Interstitial Pneumonia.

As used herein, the term “HRCT” refers to high-resolution CT (HRCT).

As used herein, the term “ILA” refers to asymptomatic interstitial lung abnormalities.

As used herein, the term “IPF” refers to idiopathic pulmonary fibrosis.

As used herein, the term “PBMC” refers to peripheral blood mononuclear cell.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions disclosed herein leads to the elimination of a sign or symptom, however, elimination is not required. Effective dosages are expected to decrease the severity of a sign or symptom. A sign is an objective indication of a medical condition that is observable or detectable by a medical professional or lay person (e.g. family member) (for example, with respect to fibrotic pulmonary disease, signs include, but are not limited to, changes in body weight, changes in body temperature and the presence of a fibrotic lesion in one or both lungs detectable by radiography).

A symptom is an indication of disease that may be a sign but may also be exclusively observable or subjectively experienced by the subject (for example, with respect to fibrotic pulmonary disease, symptoms may include but are not limited to, a dry or hacking cough, a sore throat, a tight chest, shortness of breath, and a feeling of exhaustion or malaise).

In one aspect, the terms “co-administered” and “co-administration” as relating to a subject refer to administering to the subject a compound of the invention or salt thereof along with a compound that may also treat the disorders or diseases contemplated within the invention. In one embodiment, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary and topical administration.

A “disease” as used herein is a state of health of an animal or subject wherein the animal or subject cannot maintain homeostasis, and wherein if the disease is not ameliorated then the animal's or subject's health continues to deteriorate.

A “disorder” as used herein in an animal is a state of health in which the animal or subject is able to maintain homeostasis, but in which the animal's or subject's state of health is less favorable than it would be in the absence of the disorder. Left untreated, a disorder does not necessarily cause a further decrease in the animal's or subject's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “fibrotic lung disease” or “fibroid lung disease” or “pulmonary fibrosis” or “scarring of the lung” refers to a group of diseases characterized by the formation or development of excess fibrous connective tissue (fibrosis) in the lungs. Symptoms of pulmonary fibrosis are mainly: shortness of breath, particularly with exertion; chronic dry, hacking coughing; fatigue and weakness; chest discomfort; and loss of appetite and rapid weight loss. Pulmonary fibrosis may be a secondary effect of other diseases, most of them being classified as interstitial lung diseases, such as autoimmune disorders, viral infections or other microscopic injuries to the lung. Pulmonary fibrosis can also appear without any known cause (“idiopathic”). Idiopathic pulmonary fibrosis is a diagnosis of exclusion of a characteristic set of histologic/pathologic features known as usual interstitial pneumonia (UIP).

Diseases and conditions that may cause pulmonary fibrosis as a secondary effect include: inhalation of environmental and occupational pollutants (asbestosis, silicosis and gas exposure); hypersensitivity pneumonitis, most often resulting from inhaling dust contaminated with bacterial, fungal, or animal products; cigarette smoking; connective tissue diseases such as rheumatoid arthritis, SLE; scleroderma, sarcoidosis and Wegener's granulomatosis; infections; medications such as amiodarone, bleomycin (pingyangmycin), busulfan, methotrexate, apomorphine and nitrofurantoin; and radiation therapy to the chest.

As used herein, a “subject in need thereof” is a subject suffering from fibrotic lung disease relative to the population at large. For example, the subject is a patient who is or is about to be administered with comprising administering to the subject an effective amount of a therapeutic agent. For example, the subject is asymptomatic and is at risk of developing the fibrotic lung disease. A “subject” includes a mammal. The mammal can be e.g., any mammal, e.g., a human, primate, bird, mouse, rat, fowl, dog, cat, cow, horse, goat, camel, sheep or pig. Preferably, the mammal is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

Pharmaceutically acceptable carriers of the disclosure include, but are not limited to, pharmaceutically acceptable materials, compositions or carriers, such as a liquid or solid fillers, stabilizers, dispersing agents, suspending agents, diluents, excipients, thickening agents, solvents or encapsulating materials, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

Suitable forms for administration include forms suitable for systemic administration, oral administration, for example by a capsule or tablet. Once formulated, the compositions of the disclosure can be administered directly to the subject.

The term “prevent,” “preventing” or “prevention,” as used herein, means avoiding or delaying the onset of symptoms associated with a disease or condition in a subject that has not developed such symptoms at the time the administering of an agent or compound commences.

### Compounds and Compositions

In some embodiments, compounds known to be useful in treating pulmonary fibrosis are useful within the methods of the invention. Non-limiting examples of such compounds are pirfenidone (5-methyl-1-phenylpyridin-2-one, or a salt or solvate thereof) and nintedanib (methyl (3Z)-3-{[(4-{methyl[(4-methylpiperazin-1-yl)acetyl]amino}phenyl)amino](phenyl)methylidene}-2-oxo-2,3-dihydro-1 H-indole-6-carboxylate, or a salt or solvate thereof).

In some embodiments, the subject identified as having MUC5B promoter polymorphism rs35705950 is administered a compound contemplated within the disclosure. In some embodiments, the subject is a mammal. In other embodiments, the mammal is a human.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation. Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

A suitable dose of a compound of the disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

In some embodiments of the methods of the disclosure, the therapeutic agent comprises pirfenidone. In some embodiments, the effective dosage is administered orally as a capsule or a tablet. In some embodiments, including those embodiments wherein the therapeutic agent comprises pirfenidone, the effective dosage is about 2400 mg/day. In some embodiments, the effective dosage is administered according to an escalating dosage regimen. In some embodiments, including those embodiments wherein the therapeutic agent comprises pirfenidone, the escalating dosage regimen comprises (a) administering to the subject about 800 mg of pirfenidone per day for a first week; (b) administering to the subject about 1600 mg of pirfenidone per day for a second week; and (c) administering to the subject about 2400 mg of pirfenidone per day for the remainder of the treatment. In some embodiments, including those embodiments wherein the therapeutic agent comprises pirfenidone, the escalating dosage regimen comprises (a) administering to the subject a capsule or tablet comprising about 250 mg of pirfenidone three times a day for a first week; (b) administering to the subject two capsules or tablets comprising about 250 mg of pirfenidone three times a day for a second week; and (c) administering to the subject three capsules or tablets comprising about 250 mg of pirfenidone three times a day for the remainder of the treatment. In some embodiments of the escalating dosage regimen, the capsule or tablet comprises 267 mg of pirfenidone.

In some embodiments of the methods of the disclosure, the therapeutic agent comprises nintedanib. In some embodiments, the effective dosage is administered orally as a capsule or a tablet. In some embodiments, including those embodiments wherein the therapeutic agent comprises nintedanib, the effective dosage is about 300 mg/day. In some embodiments, the effective dosage is about 150 mg administered twice per day, wherein the daily doses are administered about 12 hours apart from one another. In some embodiments, including those embodiments wherein the therapeutic agent comprises nintedanib, the effective dosage is about 200 mg/day. In some embodiments, the effective dosage is about 100 mg administered twice per day, wherein the daily doses are administered about 12 hours apart from one another. In some embodiments, including those embodiments wherein the therapeutic agent comprises nintedanib, the effective dosage is administered according to a modified or interrupted dosage regimen. In some embodiments, the modified or interrupted dosage regimen comprises (a) administering to the subject about 300 mg of nintedanib per day until the subject presents an elevated level of liver enzymes compared to a control level of liver enzymes; (b) administering to the subject about 200 mg of nintedanib per day until the subject presents the control level of liver enzymes; and (c) administering to the subject about 300 mg of nintedanib per day for the remainder of the treatment; wherein the control level of liver enzymes is a level detected in the subject prior to an initiation of the treatment. In some embodiments, including those embodiments wherein the therapeutic agent comprises nintedanib, the modified or interrupted regimen comprises (a) administering to the subject a capsule or tablet comprising about 150 mg of nintedanib twice per day until the subject presents an elevated level of liver enzymes compared to a control level of liver enzymes; (b) administering to the subject two capsules or tablets comprising about 100 mg twice per day until the subject presents an elevated level of liver enzymes compared to a control level of liver enzymes; and (c) administering to the subject a capsule or tablet comprising about 150 mg of nintedanib twice per day for the remainder of the treatment; wherein the control level of liver enzymes is a level detected in the subject prior to an initiation of the treatment.

In some embodiments, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition.

It is also noted that the term “comprising” is intended to be open and permits but does not require the inclusion of additional elements or steps. When the term “comprising” is used herein, the terms “consisting essentially of” and “consisting of” are thus also encompassed and disclosed. Throughout the description, where compositions or combinations are described as having, including, or comprising specific components or steps, it is contemplated that compositions or combinations also consist essentially of, or consist of, the recited components. Similarly, where methods or processes are described as having, including, or comprising specific process steps, the processes also consist essentially of, or consist of, the recited processing steps.

All publications and patent documents cited herein are incorporated herein by reference as if each such publication or document was specifically and individually indicated to be incorporated herein by reference.

### EXAMPLES

In order that the invention disclosed herein may be more efficiently understood, examples are provided below. It should be understood that these examples are for illustrative purposes only and are not to be construed as limiting the invention in any manner.

### Example 1: Genetic Background of Asymptomatic Siblings of FIP Subjects

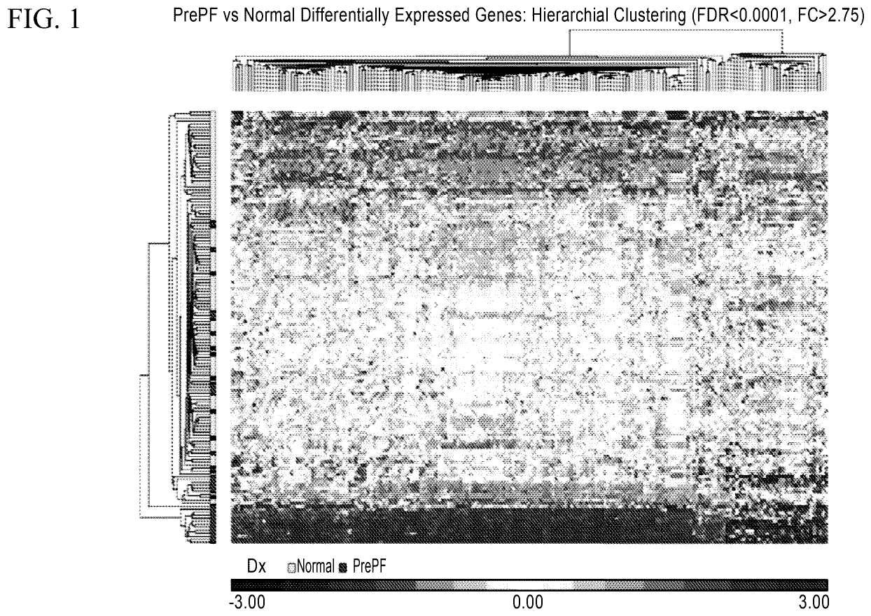

Asymptomatic siblings (>50 years old) of patients with established FIP underwent HRCT scan of the chest. HRCT scans were assessed for FILD by blinded thoracic radiologists; when possible, specific radiographic patterns were identified. PBMCs RNA and DNA were isolated. Genotyping for rs35705950 and microarray analysis were performed (SurePrint G3 Human Gene Expression Microarray). Data were analyzed using Partek Genomics Suite and RStudio. Four-hundred eighty-eight FIP siblings from 271 families were evaluated, 25 HRCT scans were excluded due to technically inadequacy, leaving 463 to be interpreted. Of these, 19% (n=88) met criteria for FILD. A subset of the positive FILD scans (n=58) were evaluated for specific interstitial patterns: the predominant radiographic finding was Usual Interstitial Pneumonia (UIP), documented as possible (n=37), probable (n=6), or definite (n=5) in 82.8% of these cases. DNA was available for 443 subjects (358 without and 85 with FILD). The minor allele (T) frequency (MAF) of rs35705950 was higher among those with evidence of FILD (MAF=0.29) than among those with normal appearing HRCT scans (MAF=0.21, p=0.005). The rs35705950 variant was associated with the presence of FILD (OR=1.90, 95% CI 1.10-3.30, p=0.02), and FILD was associated with age (OR=1.09, 95% CI 1.06-1.12, p=7.24×10-9), male sex (OR=1.81, 95% CI 1.04-3.16, p=0.04), and history of smoking (OR=1.94, 95% CI 1.11-3.40, p=0.02). Microarray analysis on PBMC RNA from 40 subjects with FILD and 105 unaffected siblings revealed 1,272 differentially expressed genes (FDR<0.05, fold-change>2); hierarchical clustering performed on the top 194 differentially expressed probes illustrates segregation of FELD subjects from unaffected siblings (FIG. 1).

### Example 2: Role of MUC5B in Pathogenesis of IPF

Common genetic variants play major and similar roles in the development of both familial and sporadic IPF (Table 3), indicating a similar etiology for familial and sporadic IPF. A common gain-of-function MUC5B promoter variant rs35705950 is a strong risk factor (genetic and otherwise), accounting for at least 30% of the total risk of developing IPF (10) confirmed in 10 independent studies, including a GWAS (OR for T (minor) allele=4.51; 95% CI=3.91-5.21; P=7.21×10-95); 3) rs35705950 may be used to identify individuals with PrePF and is predictive of radiographic progression of PrePF. MUC5B promoter variant rs35705950 is present in over 50% of non-Hispanic white (NHW) patients with IPF and is associated with unique biological and clinical IPF phenotypes. PrePF can be predicted using a combination of clinical risk factors, the MUC5B promoter variant rs35705950, and a panel of biomarkers.

TABLE 1
Common IPF risk variants identified by targeted sequencing of risk loci in 3,642 IPF cases and 4,442 unaffected controls

| | Common | Nearest | | Minor | MAF in | OR Aa vs AA | OR aa vs AA | |
|---|---|---|---|---|---|---|---|---|
| Chrm | Variant | Gene | Annotation^{a} | Allele | cases | (95% CI) | (95% CI) | P^{b} |
| 3q26 | rs2293607 | TERC | 3′ UTR | C | 0.2999 | 1.30 (1.18-1.43) | 1.79 (1.49-2.15) | 9.11 × 10⁻¹³ |
| 4q22 | rs2609260 | FAM13A | Intronic | C | 0.2289 | 1.35 (1.22-1.50) | 1.96 (1.56-2.47) | 1.03 × 10⁻¹³ |
| 5p15 | rs4449583 | TERT | Intronic | T | 0.2641 | 0.68 (0.62-0.75) | 0.46 (0.39-0.55) | 2.67 × 10⁻²⁵ |
| 6p24 | rs2076295 | DSP | Intronic | G | 0.5428 | 1.27 (1.14-1.42) | 2.08 (1.83-2.37) | 1.11 × 10⁻²⁹ |
| 7q22 | rs6963345 | ZKSCAN1 | Intronic | A | 0.4444 | 1.35 (1.22-1.50) | 1.73 (1.51-1.99) | 1.89 × 10⁻¹⁵ |
| 10q24 | rs2488000 | OBFC1 | Intronic | T | 0.08 | 0.70 (0.62-0.79)^{c} | | 7.13 × 10⁻⁹ |
| 11p15 | rs35705950 | MUC5B | Promoter | T | 0.3533 | 5.45 (4.91-6.06) | 18.68 (13.34-6.17) | 9.60 × 10⁻²⁹⁵ |
| 13q34 | rs1278769 | AK025511 | 3′ UTR | A | 0.1996 | 0.77 (0.70-0.85) | 0.69 (0.56-0.86) | 7.48 × 10⁻⁸ |
| 15q15 | rs35700143 | IVD | — | C | 0.4118 | 0.76 (0.68-0.84) | 0.63 (0.55-0.71) | 3.44 × 10⁻¹² |
| 19p13 | rs12610495 | DPP9 | Intronic | G | 0.3398 | 1.22 (1.11-1.35) | 1.59 (1.36-1.87) | 3.11 × 10⁻⁹ |

OR, odds ratio. The minor allele is defined as the minor allele in the combined case and control group.
^{a}Based on SNPDOC;
^{b}P value adjusted for sex;
^{c}OR resulting from dominant test.

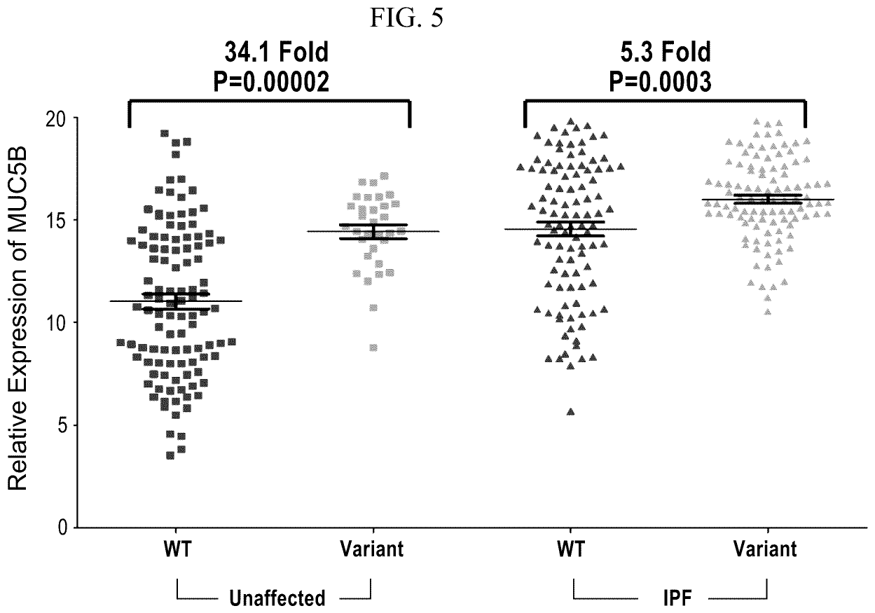

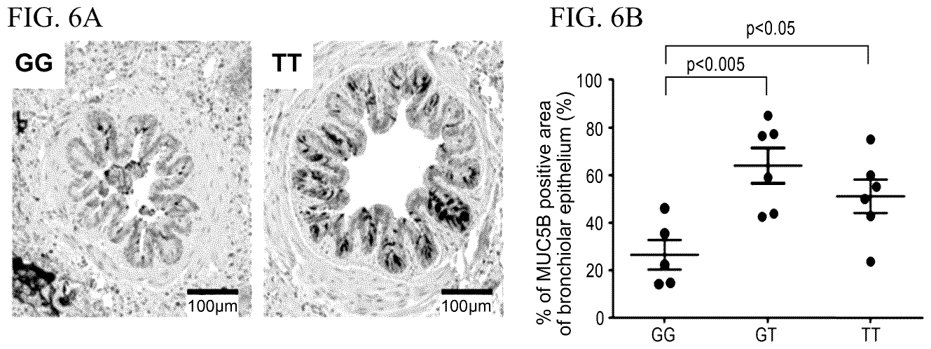

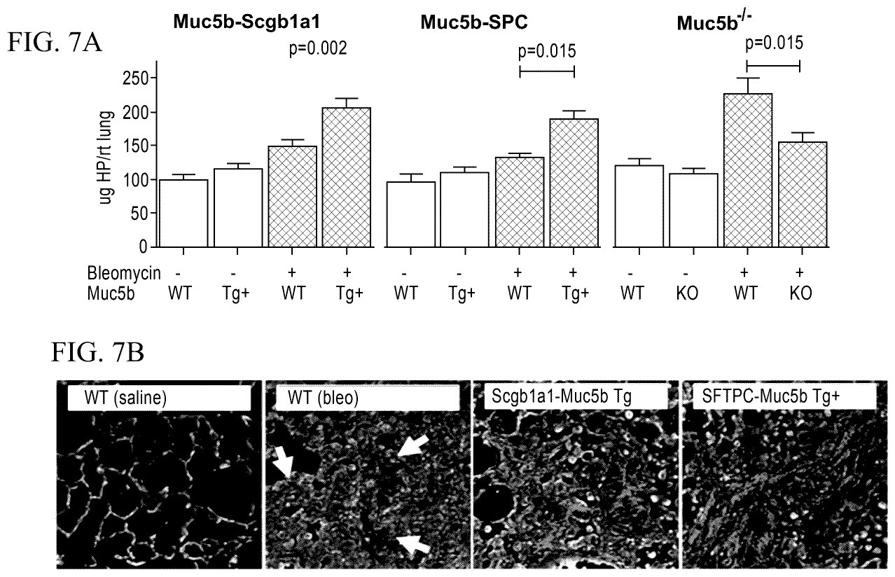

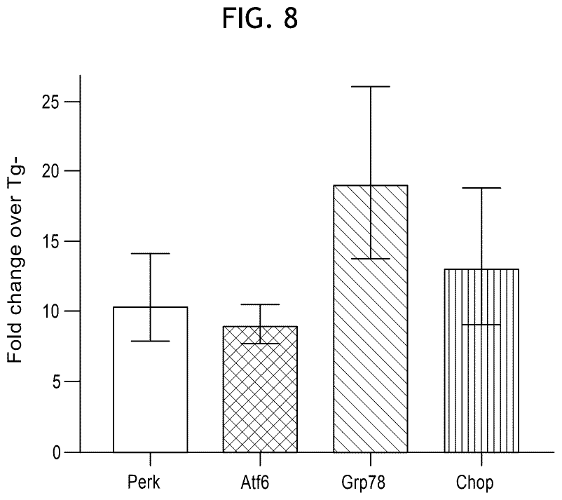

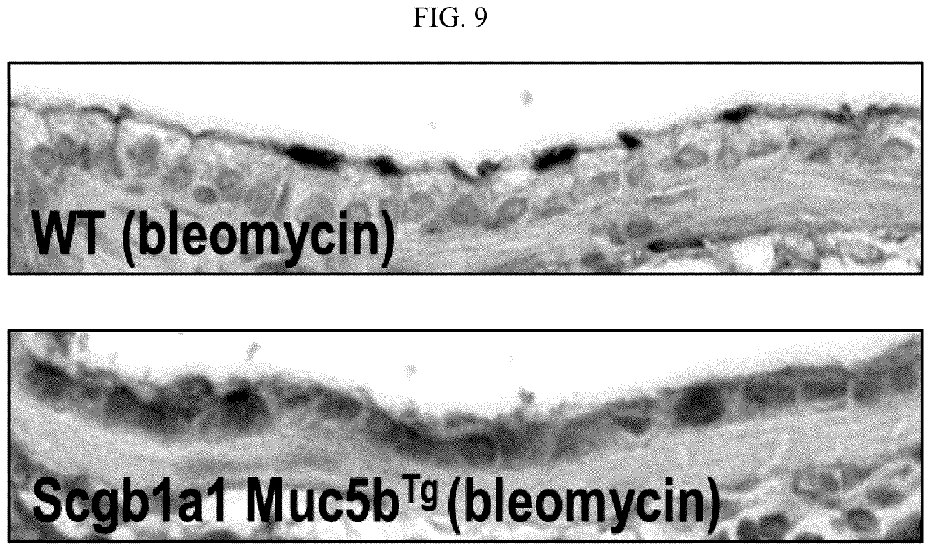

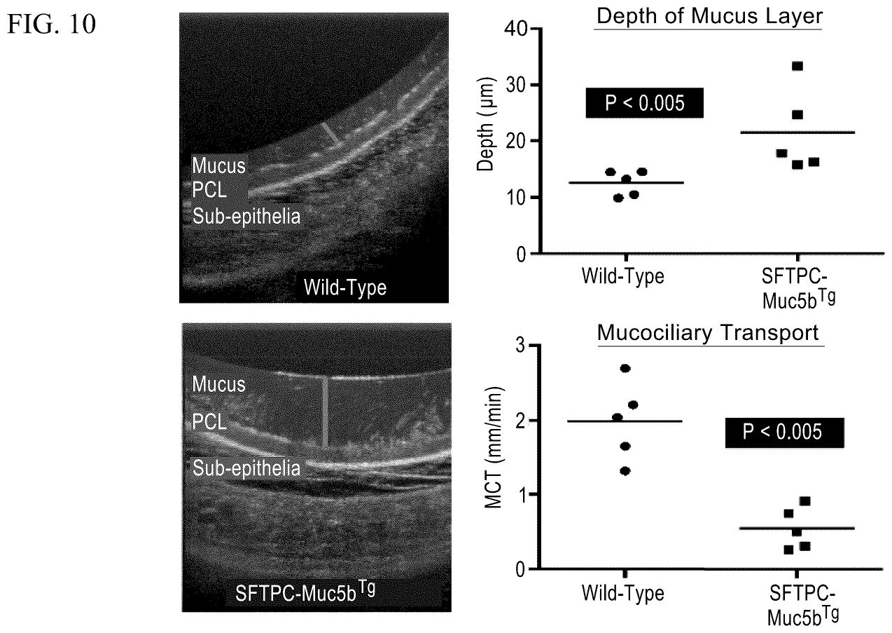

MUC5B is predicted is involved in the pathogenesis of IPF. FIG. 5 shows that MUC5B promoter variant is associated with enhanced MUC5B expression in both unaffected subjects and in patients with IPF and in IPF, MUC5B message and protein are expressed in bronchoalveolar epithelia (FIG. 6) and honeycomb cysts. In mice, the concentration of Muc5b is directly related to the fibroproliferative response to bleomycin (FIG. 7), Muc5b protein is expressed in the injured lung following bleomycin challenge, and enhanced production of Muc5b in mice appears to initiate endoplasmic reticulum (ER) stress in peripheral airways (FIGS. 8 and 9). Preliminary studies, also show that mucociliary clearance is decreased in mice that overexpress Muc5b (SFTPC-Muc5b^{Tg}) and in humans with IPF (FIG. 10).

Interstitial lung abnormalities on HRCT scans show asymptomatic relatives of patients with familial IPF and in the elderly. Similar to patients with IPF, interstitial lung abnormalities in asymptomatic subjects are associated with advanced age, cigarette smoking, reduced lung volume and decreased exercise tolerance. Moreover, the MUC5B promoter variant rs35705950 is associated with a higher prevalence of interstitial lung abnormalities on HRCT scan and is predictive of radiographic progression. Suggesting that interstitial lung abnormalities on HRCT scan are a precursor of IPF. However, interstitial lung abnormalities are not specific and include non-fibrotic and fibrotic HRCT defects, and consequently, the prevalence of interstitial lung abnormalities (>5% in the general population ≥50 years of age is orders of magnitude higher than IPF.

To address the non-specificity of interstitial lung abnormalities, a novel entity—Preclinical Pulmonary Fibrosis (PrePF) was used. PrePF is reported more frequently among smokers and in families with two or more cases of pulmonary fibrosis. In the Framingham population, data shows that PrePF is present in 1.8% of the general population ≥50 years of age (in contrast, interstitial lung abnormalities were seen in 6.7%) and that the MUC5B promoter variant rs35705950 is predictive of those with PrePF (OR=6.3 per allele [95% CI 3.1-12.7). As shown herein, among asymptomatic first-degree family members of familial interstitial pneumonia (FIP) 14% have fibrotic interstitial changes on CT scan and 35% have interstitial abnormalities on transbronchial biopsy. Moreover, in the Framingham population, it is shown that rs35705950 is predictive of radiographic progression of PrePF (OR=2.8 per allele [95% CI 1.8-4.4];) which is associated with a greater FVC decline (P=0.0001) and an increased risk of death (HR=3.7 [95% CI 1.3, 10.7]; P=0.02), indicating that in addition to having radiographic features of IPF, PrePF has similar risk factors (age, gender, smoking, and MUC5B variant) and a progressive clinical course. While the MUC5B promoter variant is predictive of PrePF, rs35705950 is present in =19% (minor allele frequency (MAF)=0.09) of the NHW population, however IPF occurs infrequently (<0.1%). Thus, additional biomarkers may be used in combination with rs35705950 identify PrePF within at-risk populations.

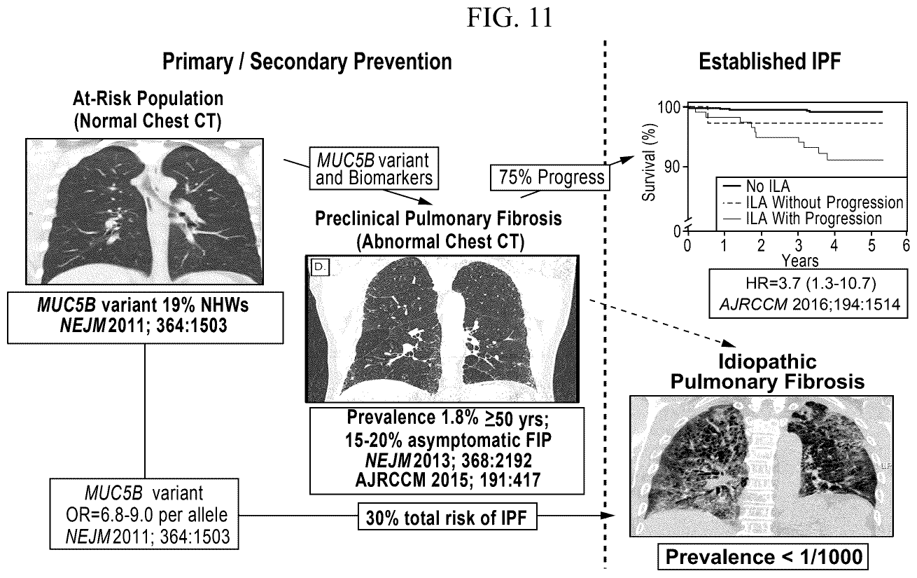

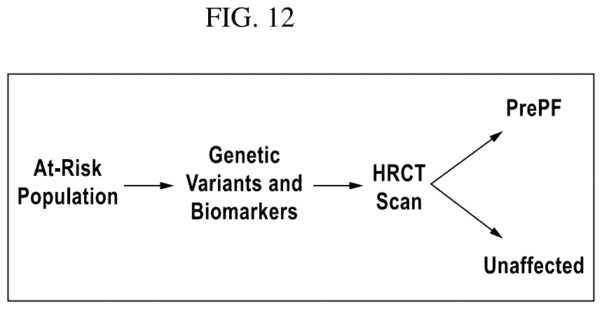

The data provided herein suggest that 1) IPF is under-diagnosed; 2) PrePF is prevalent in at-risk populations; 3) approximately 75% of the cases of PrePF are progressive; 4) radiographic progression of PrePF is associated with increased morbidity and mortality; and 5) MUC5B variant rs35705950, peripheral blood biomarkers, clinical/biological, and radiographic screening should be useful in identifying those with PrePF (FIG. 11). While IPF takes years to develop, most patients with IPF are diagnosed in the advanced stage when little can be done to influence survival. Once the lung has undergone remodeling, the non-compliant, stiff lung matrix causes additional remodeling through activation of myofibroblasts, resulting in a feed-forward loop of lung remodeling. Earlier diagnosis of IPF detects subjects with a lower burden of fibrotic lung disease.

This disclosure provides a strategic approach to screening for early forms of IPF needs to be established (FIG. 11). While the MUC5B promoter variant is predictive of PrePF (defined as chest HRCT consistent with probable or definite fibrosis (e.g., bilateral subpleural reticular changes, honeycombing, or traction bronchiectasis) occurring in asymptomatic subjects ≥40 years of age that emerge from at-risk populations), the MUC5B promoter variant is present in ≈19% of the NHW population and IPF occurs infrequently (<0.1%). To study at-risk populations (asymptomatic siblings ≥40 years of age of patients with family or sporadic IPF), identification of genetic variants and biomarkers that increase the yield of patients with PrePF are used to establish screening tools and approaches that identify early stages of IPF. This approach changes the way IPF is diagnosed and treated, and is critical to developing interventions to prevent PrePF progression to established IPF. The methods provided in this disclosure fundamentally alter the clinical approach to patients with IPF from palliative to preventive (FIG. 10).

### Example 3: Predictive Biomarker Profile for Established IPF

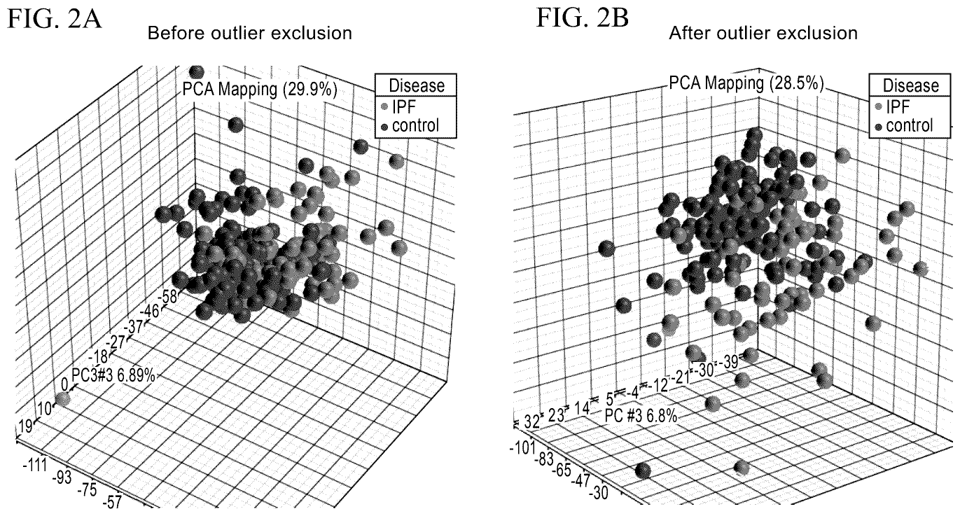

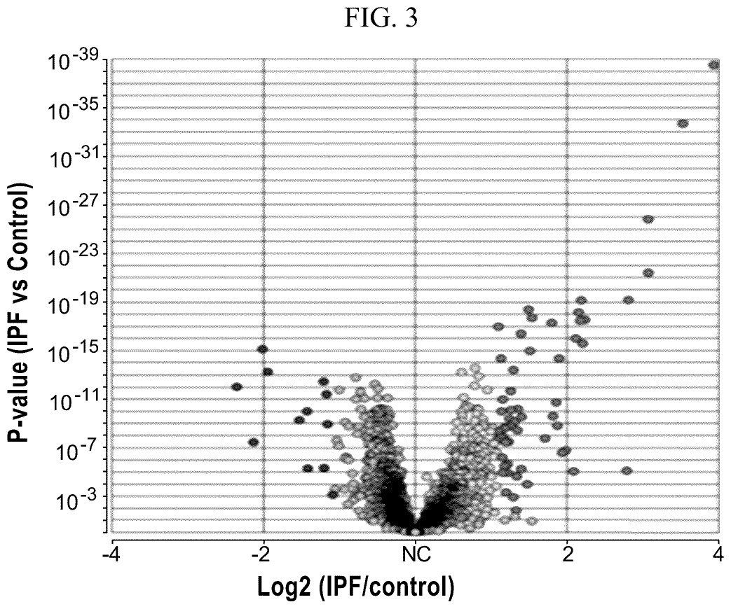

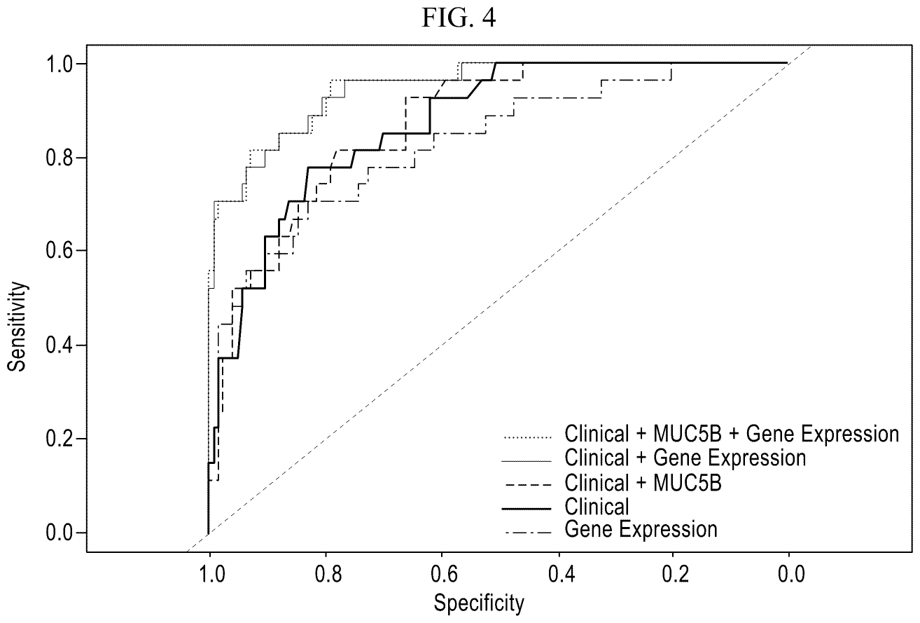

To address the development of a peripheral blood biomarker profile for IPF, an assay of the expression levels of >3700 plasma proteins was performed on plasma from 70 patients with established IPF and 70 controls. After controlling for multiple comparisons and appropriate co-variables, 57 proteins were up-regulated >1.5-fold (including surfactant proteins, MMP7, and C3) in the plasma of patients with IPF and 12 were significantly down-regulated (FIG. 2).

### Example 4: Predictive Biomarker Profile for Early IPF

To evaluate a predictive biomarker profile in cases of preclinical pulmonary fibrosis (PrePF) derived from families with familial IPF (≥2 cases of IPF in a family), HRCT scans were performed on 496 asymptomatic family members ≥40 years of age previously phenotyped as unaffected from 263 families with familial IPF. PrePF, consistent with the operational definition (defined as abnormalities on chest HRCT consistent with probable or definite fibrosis (e.g., bilateral subpleural reticular changes, honeycombing, or traction bronchiectasis) occurring in asymptomatic subjects ≥40 years that emerge from at-risk populations), was present in 77 (15.5%) of 496 asymptomatic individuals from families with familial IPF. The minor allele frequency (MAF) of the MUC5B promoter variant was 0.29 in those with PrePF versus 0.21 in those without fibrosis (P=0.025). Preliminary analysis of PBMC gene expression profiles evaluated by microarrays from 38 cases of PrePF and 187 subjects without fibrosis identified 16 genes significantly differentially expressed between the two groups (p-value <0.05 and >1.5 fold change). Among genes differentially expressed in PrePF are those involved in innate immunity and inflammatory responses (SIGLEC14), antibacterial effects (ADM2), growth and motility (TSPAN5), and protein phosphorylation (CAMKK1). Moreover, PBMC gene expression appears to contribute to the ability to predict PrePF in an at-risk population (FIG. 3).

Additionally, RNA-sequencing analysis was performed on 40 PrePF subjects and 80 subjects with a normal HRCT scan. Sequencing of the polyA-enriched libraries was prepared using Illumina TrueSEQ reagents and multiplexing 10 samples on each lane of HiSEQ4000 to obtain on average 35-40 million reads per sample. This high coverage allows for the consideration of a broad dynamic range of mRNA transcripts for biomarker selection. Platform selection of serum and plasma samples from the same subjects are used for proteomic analysis.

### Example 5: Biomarker Identification

To examine for association between each biomarkers and PrePF, a multivariable logistic regression model for PrePF with biomarkers and covariates is used for inclusion and a step-wise forward selection procedure is constructed. Variables stay in the model if associated at P≤0.01 after adjustment for the variables already in the model. Protein biomarkers that are significantly associated with established IPF and the top 20 differentially expressed genes in PrePF are considered for inclusion in a multivariable model. The number of potential biomarkers allowed in the joint model is restricted to approximately 20 given the number cases of PrePF expected. Secondarily, interactions between MUC5B genotype and the other biomarkers are tested for, which allow for the possibility that different biomarker profiles are diagnostic in IPF patients with/without the MUC5B risk allele.

### Example 6: Predictive Ability of Biomarkers

To test the predictive value of the combination of biomarkers associated with PrePF, the observed expression and other biomarker values from those associated with PrePF in the siblings of FIP patients is used to obtain the probability, for each sibling, having PrePF.

Following, a construct receiver operating characteristic (ROC) curves (see M. S. Pepe et al., Phases of biomarker development for early detection of cancer. Journal of the National Cancer Institute 93, 1054-1061 (2001)), is used to choose the probability threshold that maximizes the area under the ROC curve. This probability threshold is used to classify each individual as predicted to have PrePF or not, allowing calculation of the sensitivity, specificity, positive predictive value, and negative predictive value of the predictive model. The properties of the predictive model(s) in the independent set of siblings of patients with IPF are evaluated. Different aliquots are run for 10 samples for each assay at each time the assays is run in order to use those 10 samples to evaluate the need for standardization of the absolute values for each assay over time. Either the raw or standardized values, for a given model, is used to observe biomarker values in the PrePF siblings and non-PrePF siblings to obtain the probability of being in the disease group based on the model parameters developed using the FIP siblings. The thresholds identified among the FIP siblings are used to classify each individual as predicted to have PrePF or not. This categorization allows for the calculation of the sensitivity, specificity, positive predictive value, and negative predictive value of the predictive model among the siblings of independent cases of IPF to that observed in the siblings of FIP cases.

Power is calculated to detect differences between those with and without PrePF assuming 500 siblings and 10% (N=50) with PrePF. Assuming α=0.00005 (conservatively correcting for up to 1000 independent tests), we have 80% (90%) power to detect differences in protein or expression level of 0.74 (0.80) standard deviation between PrePF and unaffected siblings. These differences are larger than previously-observed protein and gene-expression levels in IPF patients and controls (see I. V. Yang et al., The peripheral blood transcriptome identifies the presence and extent of disease in idiopathic pulmonary fibrosis. PLoS One 7, e37708 (2012). With 50 PrePF and 450 unaffected, there is 90% power to bound the sensitivity of the biomarker-based classification of PrePF with a margin of error of 11% if the sensitivity is 65%, and 6.5% if the sensitivity is 95%; the margins of error for 65% and 95% sensitivity are 4.5% and 2.5%, respectively.

### Example 7: MUC5B Promoter Variant r35705950 is a Risk Factor for Rheumatoid Arthritis—Interstitial Lung Disease

### Methods

### Study Cohorts

This study included a discovery cohort and multi-ethnic replication cohorts. The discovery cohort included patients with RA, with and without ILD (RA-noILD) as assessed by chest HRCT, and controls, from the French RA-ILD network. The multi-ethnic replication cohorts were obtained from six countries (China, Greece, Japan, Mexico, the Netherlands and United States). This included patients with RA-ILD and RA-noILD patients, and controls. All cases fulfilled the 2010 European League Against Rheumatism-American College of Rheumatology (EULAR-ACR) and/or 1987 ACR revised criteria for RA. The ILD status of patients with RA was established by chest HRCT images that were centrally reviewed by experienced readers for each participating cohort. There was one cohort, the RA-noILD cases from the USA1 cohort, which was determined by self-report. The chest HRCT ILD pattern was classified as UIP, possible UIP or inconsistent with UIP according to international criteria and all readers were blinded to the clinical and genetic data. The institutional review boards at each institution approved all protocols, and all patients provided written informed consent.

### Genotyping

Genotyping of the MUC5B rs35705950 single nucleotide polymorphism (SNP) involved use of Taqman Genotyping Assays (Applied Biosystems, Foster City, CA, USA) as previously reported, by direct Sanger Sequencing or imputation from genome-wide association study data.

The additional common IPF risk variants on 3q26, 4q22, 5p15, 6p21.3, 6p24, 7q22, 10q24, 11p15.5, 13q34, 15q14-15, and 19p13 were genotyped by Taqman qPCR (Thermo Fisher Scientific, California) per the manufacturer's instructions.

### Lung Tissue Analysis

In order to determine if MUC5B was expressed in RA-ILD ling tissue, we analyzed lung tissue was analyzed from nine patients with RA-ILD undergoing lung transplantation (University of California, San Francisco) compared to six unaffected controls with ILD (NHLBI Lung Tissue Research Consortium; https://ltrcpublic.com) or concordant expression of other relevant markers of pulmonary fibrosis. The tissue was formalin fixed, paraffin embedded and cut in 4 um sections. Tissue sections were deparaffinized in xylene, followed by dehydration in series of ethanol. Following citrate buffer antigen retrieval, slides were incubated overnight with primary antibodies against MUC5B (1:4000, Santa Cruz, Dallas, TX). Secondary antibody diluted 1:1000 tagged with HRP (Life Technologies) was visualized using an Aperio CS2 slide scanner (Leica, Buffalo Grove, IL).

### Results

### Study Cohorts

This case-control genetic study included 620 RA-ILD cases, 614 RA-noILD cases and 5448 unaffected controls. The discovery cohort included 118 RA-ILD cases, 105 RAnoILD cases and 1229 unaffected controls. The multi-ethnic replication sample included 502 RA-ILD, 509 RA-noILD cases and 4219 unaffected controls.

### Characteristics of the Discovery Cohort

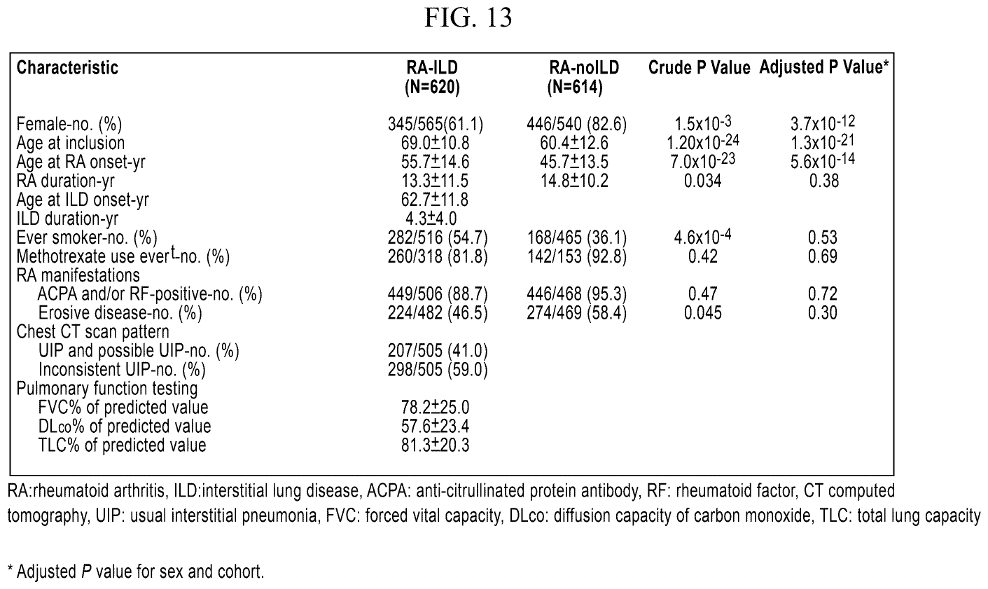

As compared with RA-noILD, patients with RA-ILD were more frequently male, older and more frequently smoked cigarettes (54.7% versus 36.1%) (FIG. 13). However, after adjusting for sex, the relationship between RA-ILD and cigarette smoking was no longer statistically significant (FIG. 13). After adjustment, RA-ILD and RA-noILD patients did not differ in rheumatoid factor (RF) and/or anti-citrullinated protein antibody (ACPA) positivity, erosive status of RA, exposure to methotrexate or the mean RA duration from diagnosis at inclusion in the cohort. Overall, 41% of patients with RA-ILD had a UIP or possible UIP HRCT pattern.

### Muc5B Promoter Variant and Risk of Rheumatoid Arthritis-Associated Interstitial Lung Disease

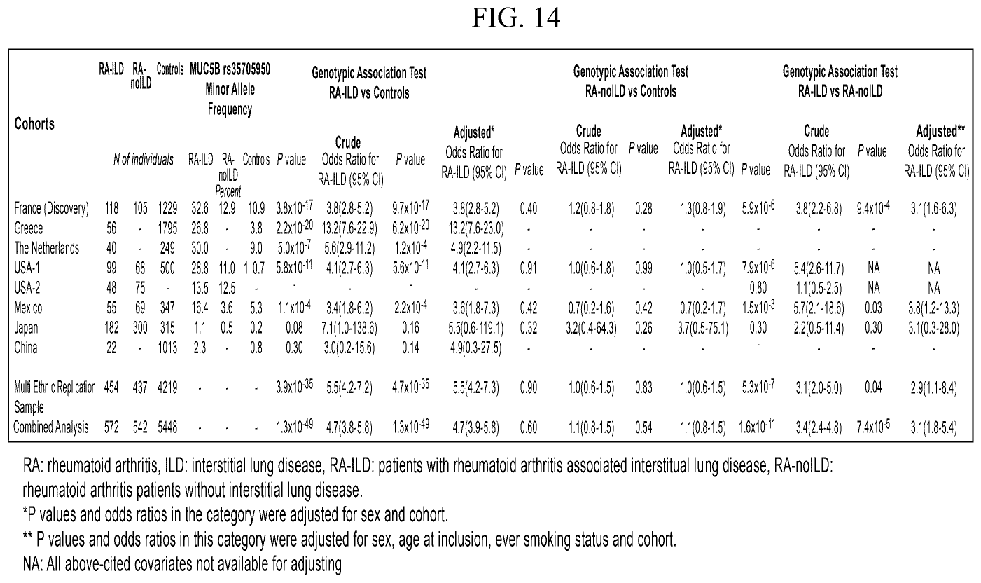

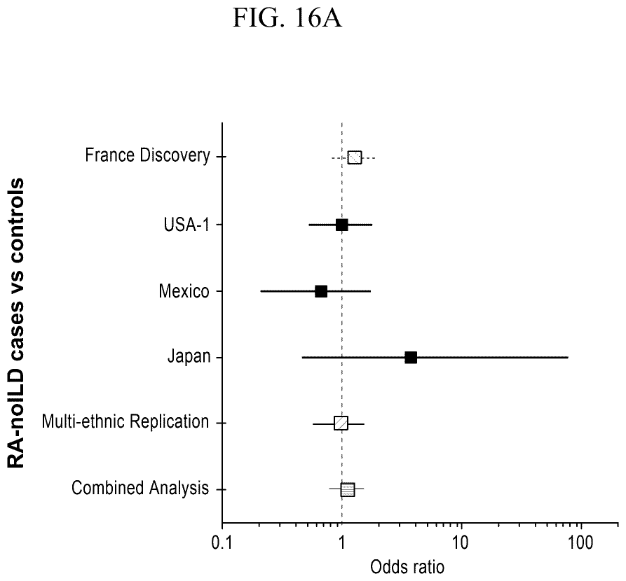

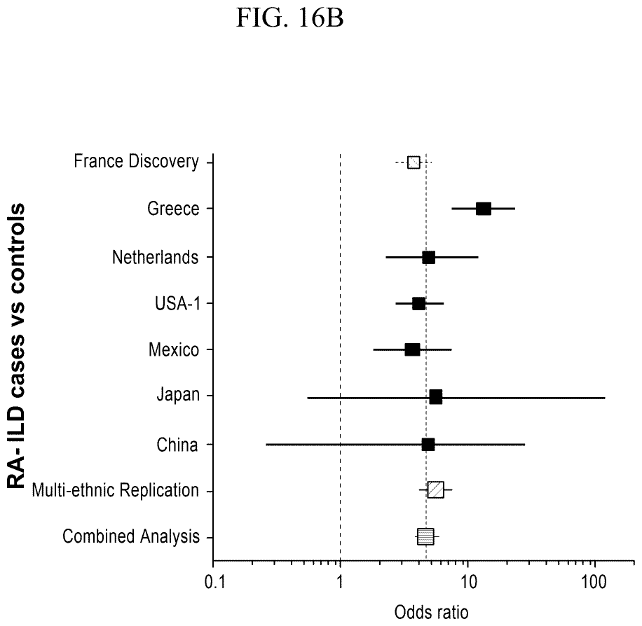

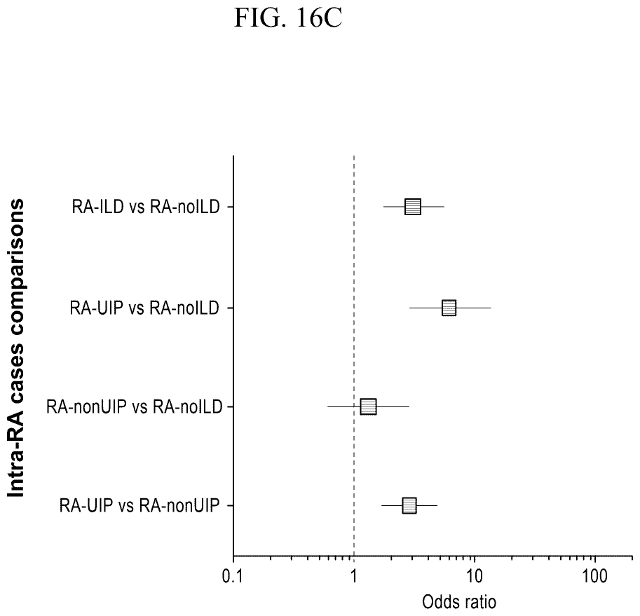

Comparison of RA-noILD and controls revealed that none of the cohorts (discovery cohort and multi-ethnic cohorts) demonstrated a significant difference in the frequency of the MUC5B promoter variant (FIG. 14; FIG. 16A), suggesting a lack of association between the MUC5B promoter variant and RA. In the discovery cohort, the minor allele frequency (MAF) of the MUC5B promoter variant was 10.9% in unaffected controls and 32.6% in cases of RAILD; this variant was in Hardy-Weinberg equilibrium (HWE) in both study groups. I In the discovery population, after controlling for sex we detected a significant association between the MUCSB promoter variant and RA-ILD when compared to non-RA controls (ORadj=3.8; 95% CI, 2.8 to 5.2; P=9.7×10-17) (FIG. 14). Similar to the discovery population, the MUCSB promoter variant was significantly over-represented among the cases of RA-ILD compared to unaffected non-RA controls in all of the multi-ethnic study case series, except in the two Asian case series (FIG. 14). Given that the MUCSB promoter variant is under-represented in Asian populations compared to non-Hispanic whites (FIG. 14; www.ncbi.nlm.nih.gov/projects/SNP/snp_ref.cgi?rs=35705950), a likely explanation, especially given the consistent point estimates, for the absence of a significant relationship between the MUCSB promoter variant and RA-ILD is that the analysis of the two Asian case series is likely underpowered. The relationship between the MUCSB promoter variant and RA-ILD in combined multi-ethnic study case series (ORadj=4.7; 95% CI, 3.9 to 5.8; P=1.3×10-49) (FIG. 14) (FIG. 16B) validated the observed association between the MUCSB promoter variant and RA-ILD in the discovery study population. In addition, the cases of RA-ILD in the study populations from Greece and USA-1 were not in HWE, suggesting (as has been observed in cases of IPF 14), that the MUCSB promoter variant and/or common variants in high or complete linkage disequilibrium with the MUCSB promoter variant should be considered as causative in these cases of RA-ILD. For the comparison with non-RA controls, the best-fitting genetic model for the three study populations (discovery population, combined multi-ethnic case series, and combined analysis) for the association of the MUCSB MUC5B RS35705950 AND RISK OF INTERSTITIAL LUNG DISEASE AMONG PATIENTS WITH RHEUMATOID ARTHRITIS

To further investigate whether the MUC5B promoter variant rs35705950 contributes to the risk of ILD among patients with RA, we compared RA-ILD and RA-noILD patients, adjusting for sex, age at inclusion and cigarette smoking. In the discovery cohort, the MUC5B variant was associated with RA-ILD (ORadj=3.1; 95% CI, 1.6 to 6.3; P=9.4×10⁻⁴), and this finding was replicated in the aggregate multi-ethnic cohort (ORadj=2.9; 95% CI, 1.1 to 8.4; P=0.04) and the combined analysis (ORadj, 3.1; 95% CI, 1.8 to 5.4; P=7.4×10⁻⁵) (FIG. 14; FIG. 16C). For the comparison of RA-ILD with RA-noILD, the best-fitting genetic model for the three study cohorts (discovery population, combined multi-ethnic case series, and combined analysis) was dominant. After adjusting for covariates, no association between tobacco smoking and the risk of ILD among patients with RA was found and no interaction of tobacco smoke exposure with the MUC5B promoter variant was observed (ORadj=0.7; 95% CI, 0.3 to 1.9; P=0.51).

MUC5B rs35705950 and UIP on HRCT Scan

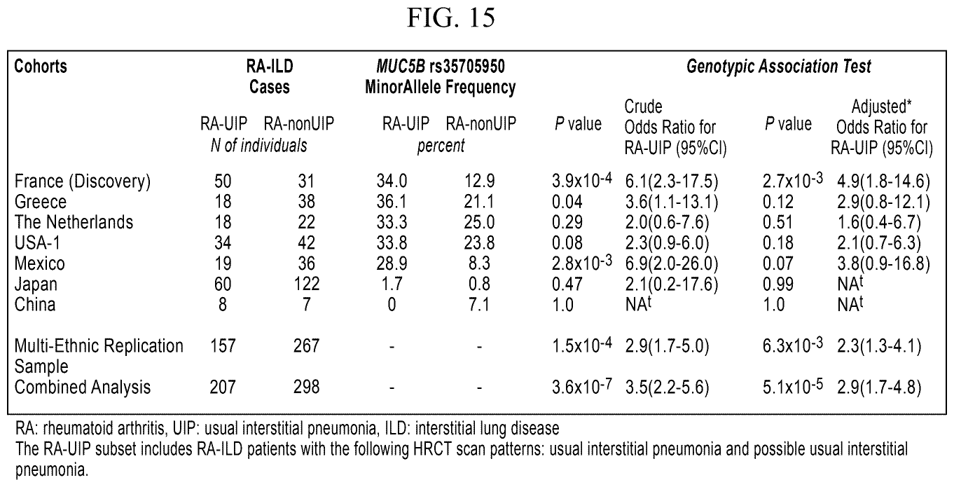

Limiting the RA-ILD cases to those with radiographic evidence of definite or possible UIP on HRCT scan, the association observed in the discovery cohort (ORadj=5.0; 95% CI, 2.1 to 12.3; P=3.0×10⁻⁴), was replicated in the combined multi-ethnic cohort (ORadj=9.2; 95% CI, 2.3 to 38.7; P=1.8×10⁻³) (FIG. 16C), and was observed in the combined cohort analysis (ORadj=6.1; 95% CI, 2.9 to 13.1; P=2.5×10⁻⁶) (FIG. 16C). In the combined analysis, the comparison of odds ratios for UIP RA-ILD vs RA-noILD (ORadj=6.1; 95% CI, 2.9 to 13.1; P=2.5×10⁻⁶) to non-UIP RA-ILD vs RA-noILD (ORadj=1.3; 95% CI, 0.6 to 2.8; P=0.46) was statistically significant (P=0.02), suggesting that the effect of the MUCSB promoter variant was restricted to the UIP RA-ILD sub-phenotype (FIG. 16C). Finally, consistent with our previous findings, the MUCSB promoter variant was found to increase the risk of developing a UIP pattern among patients with RA-ILD through a dominant model in the discovery, replication and combined analysis; the odds of having a UIP and possible UIP pattern for patients with RA-ILD carrying at least one MUCSB rs35705950 T risk allele were 2.9 times greater than individuals having the GG genotype (ORadj=2.9; 95% CI, 1.7 to 4.8; P=5.1×10-5) (FIG. 15; FIG. 16C). After adjusting for covariates, tobacco smoking exposure did not contribute to a specific HRCT pattern for RA-ILD and no interaction with the MUC5B rs35705950 variant was detected.

### Sites of MUC5B Expression in RA-ILD

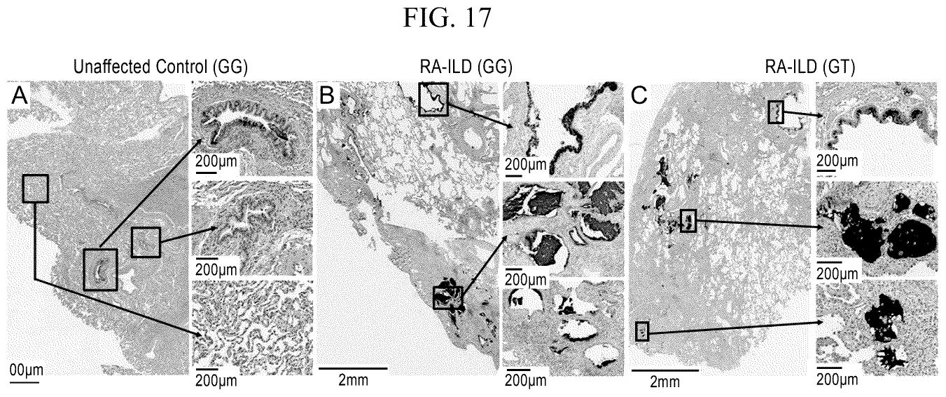

We performed immunohistochemical staining for MUC5B in nine RA-ILD lung tissue explants (5 GG and 4 GT) and 6 unaffected controls (3 GG and 3 GT). Similar to what has been reported in IPF, RA-ILD lung tissue demonstrated MUC5B in the cytoplasm of the bronchioles and in areas of microscopic honeycombing, including staining of the metaplastic epithelia lining the honeycomb cysts and the mucous within the cyst (FIG. 17). The controls demonstrated MUC5B expression in the bronchioles only. There were no obvious differences in MUC5B expression by genotype.

### Exploratory Genetic Association Study of 12 Common IPF Risk Variants in RA-ILD

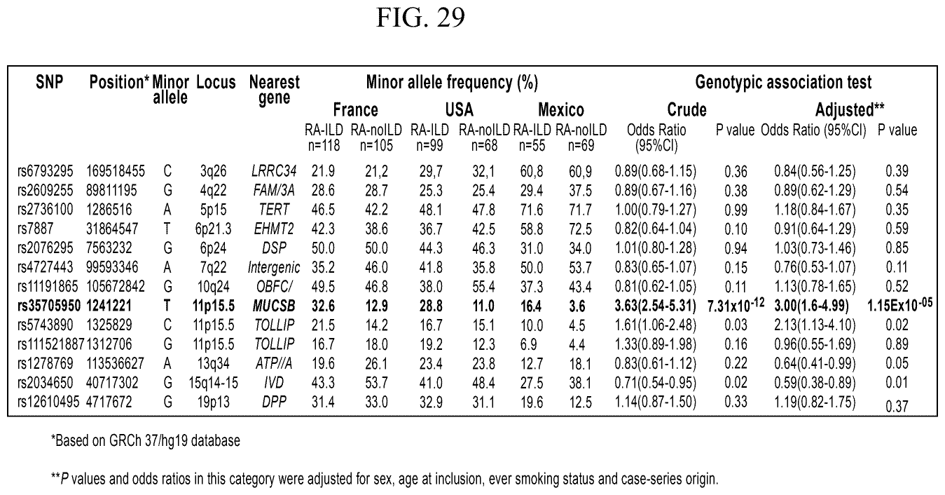

Having provided evidence for the contribution of the dominant IPF genetic risk variant, i.e. the MUCSB promoter variant, to RA-ILD, we decided to test the association of 12 additional common IPF risk variants with RA-ILD (FIG. 29). This exploratory study included 272 RA-ILD and 242 RA-noILD patients from the France, USA-1 and Mexico case series. Taking into account the relatively small sample size and related low power of detection corresponding P-values, Odds Ratio and 95% CI for the 12 candidate variants were considered as descriptive and Bonferoni correction was therefore not applied (Table 4). Comparison between RA-ILD and RA-noILD revealed that 2 common IPF risk variants, TOLLIP rs5743890 and IVD rs2034650, were significantly associated with RA-ILD. The TOLLIP rs5743890 minor allele was associated with increased risk of RA-ILD and the IVD rs2034650 minor allele was associated with decreased risk of RA-ILD (ORadj=2.13; 95% CI, 1.13 to 4.10; P=0.02 and ORadj=0.59; 95% CI, 0.38 to 0.89; P=0.01, respectively) and the directionality of these relationships is consistent with what has been observed for IPF.16,17 No association with RA-ILD was detected for the 10 other IPF risk variants (FIG. 29).

### Example 8: MUC5B Promoter Variant is Associated with Visually and Quantitatively Detected Preclinical Pulmonary Fibrosis

Better understanding and recognition of early pulmonary fibrosis is critical because medical therapies have been shown to slow progression, not to reverse or even stabilize established fibrosis—therefore, intervention before irreversible fibrosis has become extensive has the potential to improve quality of life and decrease morbidity. While IPF affects approximately 5 million people worldwide, between 1 8 and 14% of the general population ≥50 years of age have radiologic findings of undiagnosed pulmonary fibrosis. Large cohort studies indicate that interstitial lung abnormalities, postulated to represent early pulmonary fibrosis, are associated with increased mortality, and that most of these abnormalities progress over time. Members of families with 2 or more cases of pulmonary fibrosis (FIP, Familial Interstitial Pneumonia) have been identified as an “at-risk” population. In a previous study of FIP relatives, 14% had interstitial lung abnormalities on high resolution computed tomography (HRCT), and 35% had an abnormal transbronchial biopsy indicating interstitial lung disease.

HRCT provides visualization of the lung parenchyma and plays a key role in the diagnosis of the Idiopathic Interstitial Pneumonias (IIPs), including IPF. Currently, visual diagnosis by thoracic radiologists, in conjunction with multidisciplinary clinical conference, is the gold standard for diagnosing IPs. However, visual assessment is imprecise and hampered by inter-observer variation. Quantitative HRCT (qHRCT) evaluation provides measures of fibrosis extent that, in subjects diagnosed with IPF, correlate with degree of physiologic impairment at baseline, and may be more sensitive to subtle changes in disease status than routinely used physiological metrics. The design and utility of quantitative methods in the context of early forms of fibrotic ILD requires further study. Deep learning methods have been increasingly used in imaging to identify and classify CT patterns, and may be particularly valuable in detection of early lung fibrosis.

This study aims to: (1) examine risk factors, including two common fibrosis-associated genetic variants in MUC5B and TERT, for undiagnosed pulmonary fibrosis (PrePF) in FIP first-degree relatives; and (2) determine the utility of a deep learning, texture-based qHRCT method in the detection of early fibrosis in this cohort.

### Materials and Methods

### FIP Relatives Screening:

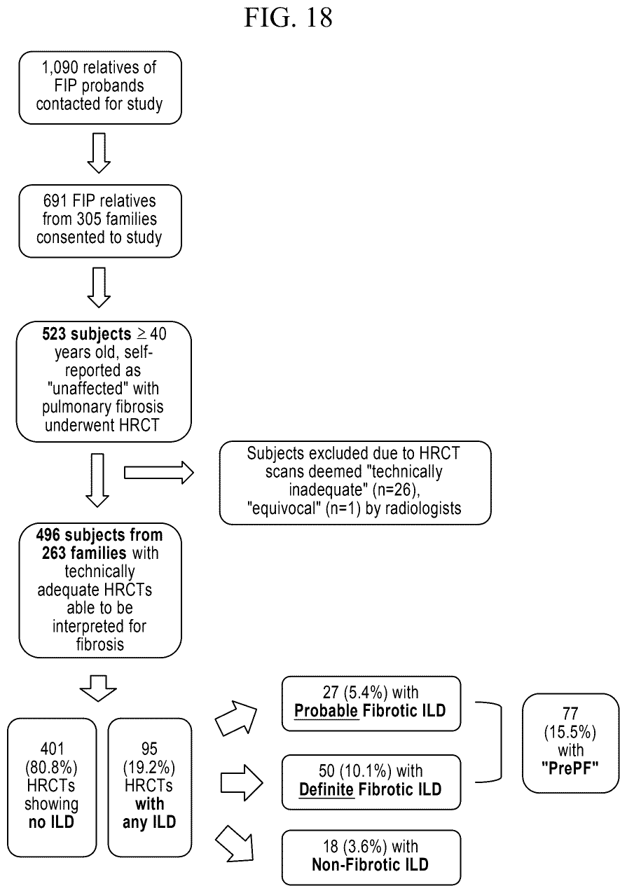

As part of a study of FIP conducted at the University of Colorado, National Jewish Health, and Vanderbilt University (COMIRB #15-1147; NJH IRB 1441a; Vanderbilt IRB #020343), non-Hispanic white (NHW) relatives of FIP patients, defined as those in families with two or more cases of pulmonary fibrosis, were contacted for enrollment. First-degree relatives without a known prior diagnosis of pulmonary fibrosis and greater than 40 years of age were offered HRCT scans of the chest and asked to undergo peripheral blood draw. Study subjects younger than 40 years of age or older than 40 years of age who reported on pre-study questionnaires to be personally affected by pulmonary fibrosis were excluded (FIG. 18).

### Visual CT Review:

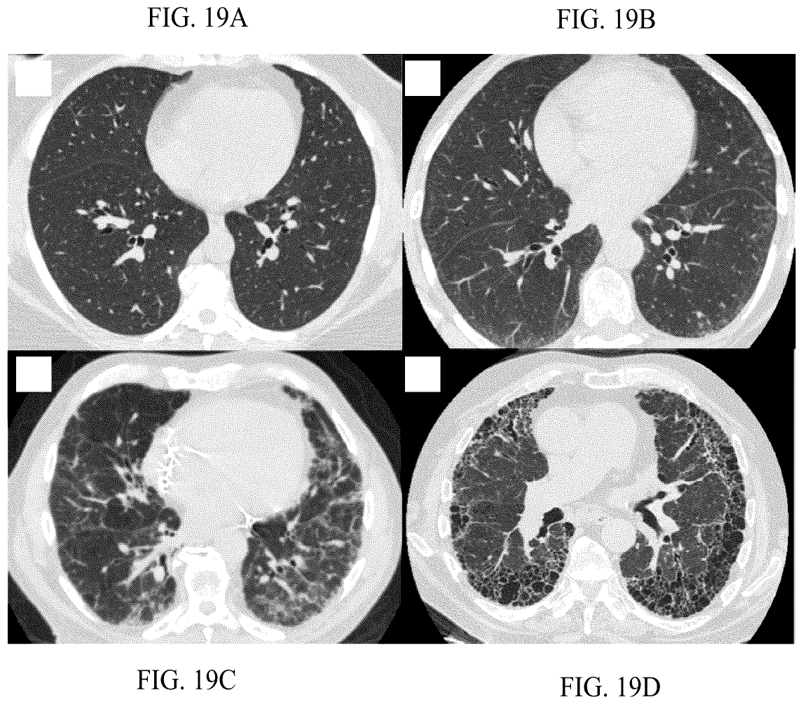

HRCT scans were interpreted by study radiologists and examined for the presence of fibrotic ILD. “PrePF” was defined as the presence of “probable” or “definite” fibrotic ILD on HRCT in FIP relatives who had no known diagnosis of pulmonary fibrosis at the time of study enrollment (FIGS. 18, 19).

### Quantitative CT:

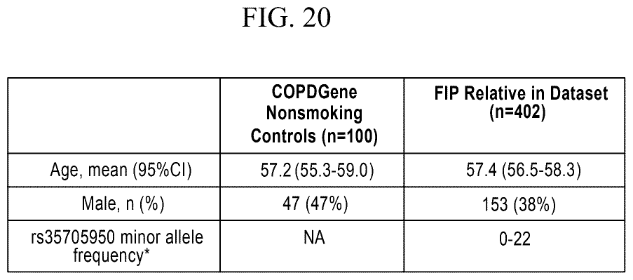

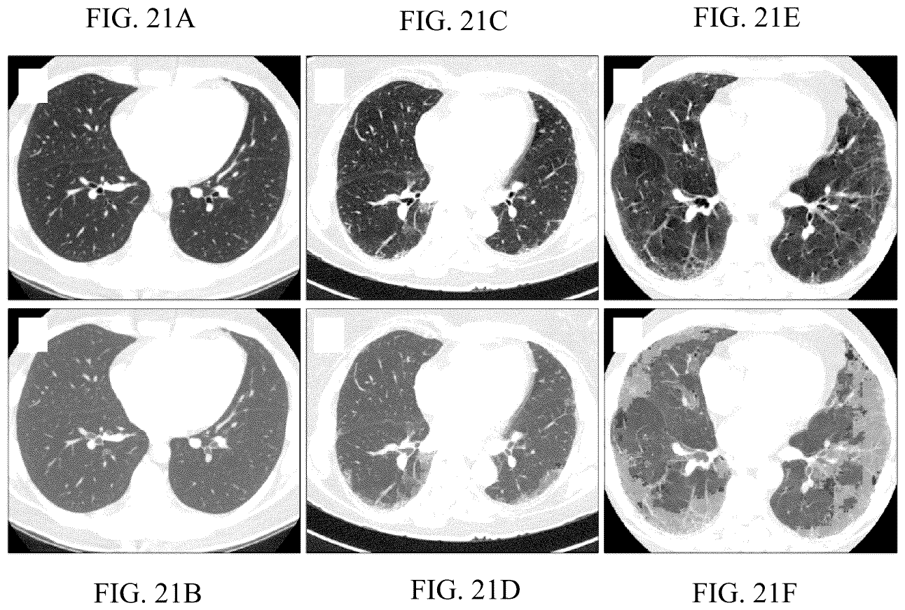

Inspiratory HRCT series with slice thickness ≤1.25 mm and spacing ≤20.0 mm were selected for quantitative analysis. This included 212 volumetric series with thin, contiguous sections (slice thickness and spacing both <=1.25 mm) and 191 non-volumetric scans (56 with slice spacing >1.25 mm and <10 mm, 65 with slice spacing of 10 mm and 70 with slice spacing=20 mm). Scans identified as technically inadequate were omitted. In addition, 100 inspiratory volumetric HRCT of never-smoking control subjects from the COPDGene cohort were analyzed (FIG. 20). The lungs were segmented in a semi-automatic fashion using open source software followed by manual editing, if necessary, performed by trained analysts. Examples of the categorization of different parts of CT scans are shown in FIG. 21. Some studies were acquired with contiguous thin axial sections while others used 1 or 2 cm intervals. Also, reconstruction kernel, a parameter that affects image sharpness and noise, was not standardized.

Fibrosis quantification on CT scans was performed using a deep learning technique, with a convolutional neural network (CNN) algorithm trained with image regions of normal and abnormal lung identified by expert radiologists. Training data and an earlier algorithm version were described previously. Here, a more complex CNN architecture was employed that classifies image regions using pixel and texture features extracted by multiple convolutional layers at different scales. Classification categories included normal lung, airways, reticular abnormality, honeycombing and ground glass. An additional category, “not normal”, was also included for lung regions not classified into any of the named categories. Further, pixels in the “not normal” category were split into two subcategories: “not normal” low density and “not normal” high density using the threshold value of −650 Hounsfield Units (HU). Subject level scores were computed as the percentage of total lung volume classified in each category. HRCT fibrosis score was defined as the sum of CNN classification scores for reticular abnormality, honeycombing, ground glass, and “not normal high density” (FIG. 21).

A simpler previously described densitometric analysis of HRCTs was also performed for comparison. Percent high attenuation area (% HAA), the percentage of total lung volume with HRCT pixel intensity greater than −600 HU and less than −250 HU, has been used as a measure of interstitial lung disease on CT.

### Statistical Analysis:

Analysis of the effect of specific alleles on PrePF risk was performed using minor allele frequency (MAF) for comparison of variant prevalence in the study groups; statistical significance was determined utilizing either a z-score test for proportions or a mixed effects logistic regression model when controlling for other clinical factors (age, sex, and history of smoking) and family [random effect]) in both dominant and log-additive models.

Distribution of qHRCT fibrosis scores was left skewed as was % HAA, and therefore these values were log transformed prior to analyses. Log of qHRCT fibrosis score (hereafter, “fibrosis score”) and log (% HAA) were compared with visual scores using ANOVA and Tukey's honest significant difference (HSD) test. To determine the ability of qHRCT scores to predict visual diagnosis of PrePF, receiver-operating characteristic (ROC) analysis was performed. Optimal threshold for discriminating visual diagnosis of fibrotic ILD was determined with Youden's method. Five-fold cross-validation was performed to test detection accuracy, sensitivity and specificity, and consistency of optimal threshold. Linear regression was performed to test association between the MUC5B genotype and qHRCT fibrosis score and log (% HAA).

A p-value of <0.05 was considered statistically significant for differences between groups as well as for associations between individual variables and outcomes in linear and logistic regression modeling. Statistical analyses were performed using RStudio (Version 0 99 473).

### Results

### Study Cohort Characteristics

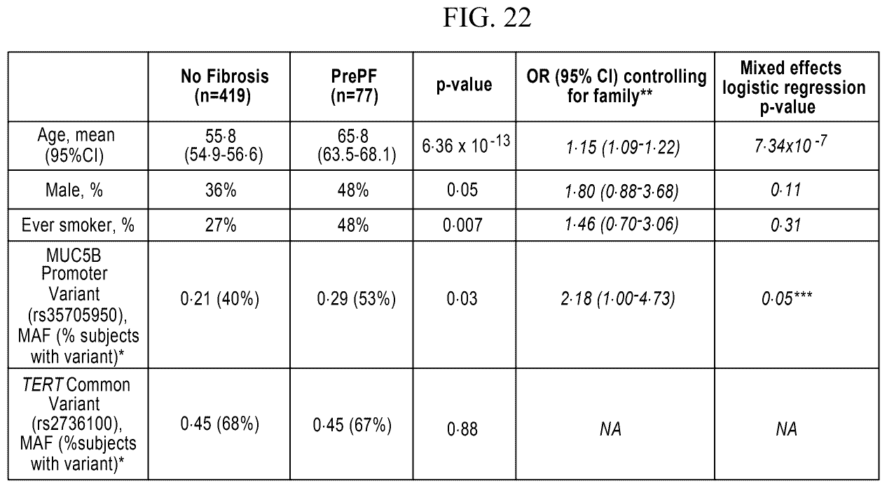

A total of 1,090 FIP relatives were contacted, and 523 eligible subjects were recruited and underwent HRCT screening (FIG. 18). Of the 523 subjects, 26 were excluded due to technical inadequacy of images and one for an equivocal consensus read by study radiologists. The remaining 496 subjects from 263 families were included in the final analyses. The mean age of study subjects was 57 years (95% CI: 56 5-58), 189 (38%) were male, and 148 (29%) were either current or former smokers. The minor allele (T) frequency of the MUC5B promoter polymorphism rs35705950 was 0 22 in this cohort; 45% of the subjects in this cohort had one or two copies of the minor allele (FIG. 22). The minor allele (C) frequency of the TERT variant rs2736100 was 0 47 in the entire cohort; 69% of the subjects in the cohort having one or two copies of the minor allele (FIG. 22).

### Prevalence of Preclinical Pulmonary Fibrosis (PrePF) in FIP Relatives

Of the 496 HRCT scans, 401 showed no CT evidence of interstitial lung disease (ILD), and 95 showed evidence of ILD, either fibrotic (27 probable and 50 definite) or non-fibrotic (n=18). Therefore, among these 496 subjects who reported being personally unaffected by pulmonary fibrosis, the PrePF prevalence was 15 5% (n=77) (FIG. 18).

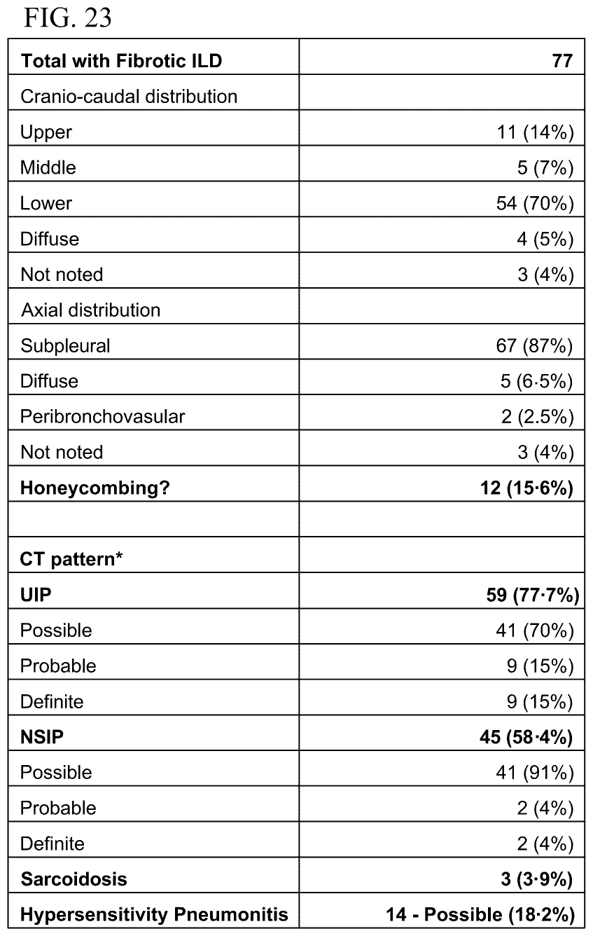

The CT patterns noted in PrePF subjects (FIG. 23) show that possible, probable, or definite UIP pattern was the most commonly considered (n=59, 77% of all PrePF cases). NSIP was considered in 45 subjects (58% of all PrePF cases). The fibrotic changes were most commonly lower-lobe predominant and subpleural in nature, consistent with a UIP pattern (FIG. 23). Non-fibrotic ILD scans, on the other hand, generally had more diffuse, upper-lobe predominant abnormalities.

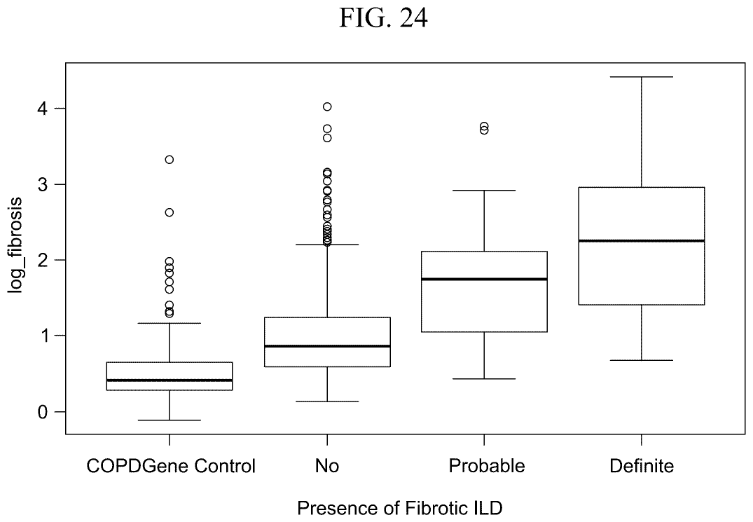

There were 402 study subjects with HRCT scans that were technically adequate for quantitative assessment. 212 of the scans had both slice thickness and spacing <=1.25 mm (thin, contiguous); of the remaining 191 scans, 56 had slice spacing >1.25 mm and <10 mm, 65 had slice spacing=10 mm, and 70 had slice spacing=20 mm. Volumetric HRCT scans on an additional 100 COPDGene subjects were included as normal controls. Fibrosis score means were significantly different (p<0.0001) across groups defined by visual diagnosis (FIG. 24). Comparison of means showed fibrosis score were significantly different comparing each group (all between-group comparisons p<0.01). Means of log (% HAA) scores were also significantly different across visual scoring groups (p<0.0001), and individual between-group comparisons showed log (% HAA) was significantly different in most comparisons (p<0.0001), except between the “probable” and “definite” visual scores (p=0.35).

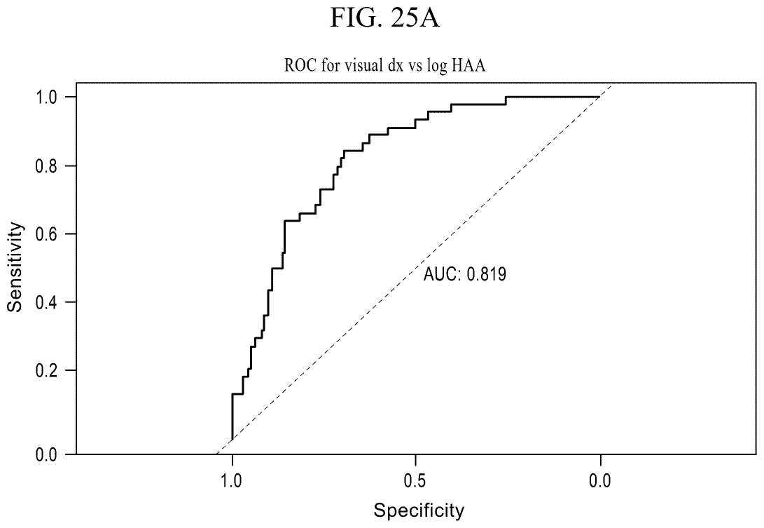

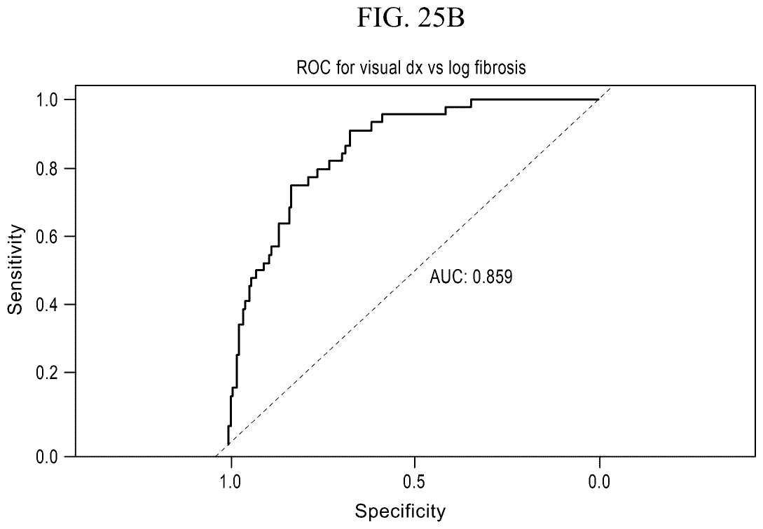

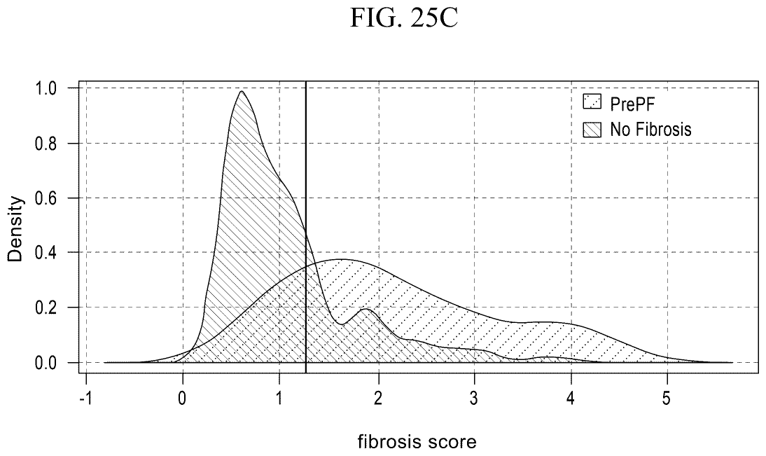

ROC analysis showed that fibrosis score discriminates subjects with visual diagnosis of PrePF (FIG. 25B). Average area under the curve (AUC) in five-fold cross validation was 0.85 (range 0.83-0.87) and average accuracy, sensitivity, and specificity in the test partitions were 0.83 (range 0.74-0.86), 0.74 (range 0.56-0.92), and 0.84 (range 0.76-0.89), respectively. Optimal threshold for fibrosis score ranged from 1.40-1.42, corresponding to 4.1% fibrotic area in examined lung. Utilizing a cutoff of 1.40 for fibrosis score on the entire dataset, the sensitivity was 74%, specificity was 82%, and accuracy was 81%; the negative predictive value of this test was 95%, exceeding its positive predictive value (42%) (FIG. 25C).

Compared to the classification achieved with the CNN as described above, ROC analysis of log % HAA had lower mean AUC 0 80 (range 0.79-0.81) and average accuracy, sensitivity, and specificity of 0.67 (range 0.63-0.70), 0.82 (range 0.75-0.91) and 0.64 (range 0.62-0.70) respectively (FIG. 25A). Optimal threshold for log % HAA ranged from 1.49-1.57. Utilizing a cutoff of 1.49 for log % HAA, the sensitivity was 88%, specificity was 55%, and accuracy was 60%; the negative predictive value of this test was 96%, exceeding its positive predictive value (26%).

### Risk Factors for PrePF

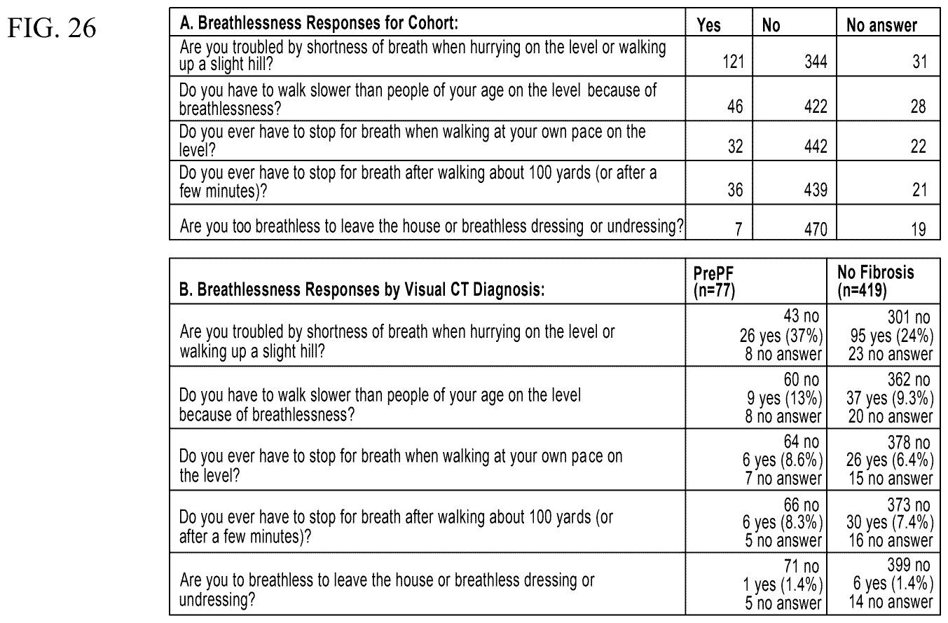

Subjects with PrePF were older (mean age 65 8 years, 95% CI 63.5-68.1) than those without fibrosis (mean age 55.8, 95% CI 54.9-56.6, p=6.36×10⁻¹³); they were also more likely to have ever smoked (43% versus 27%, p=0.007), and to be male (48% versus 36%, p=0.05). However, there was no difference in breathlessness between the PrePF and subjects without fibrosis (mean score 0.5 versus 0.6, p=0.24, FIG. 26). When fibrosis was defined by quantitative fibrosis score cutoff (1.4), there was a significant difference between groups in terms of mean breathlessness score (0.39 versus 0.78, p=0.003). Quantitative fibrosis score was positively associated with breathlessness score (p=0.001), even after controlling for age (p=1.9×10⁻⁹), male sex (p=0.7), and smoking history (p=0.8).

Screening for autoantibodies in this cohort revealed that there were no differences between PrePF and No Fibrosis subjects in terms of overall seropositivity or individual antibodies' testing in this cohort. For quantitatively defined fibrosis, there was no significant difference between groups in terms of auto-antibody testing, with similar overall seropositivity rates (11% versus 16%, p=0.30).

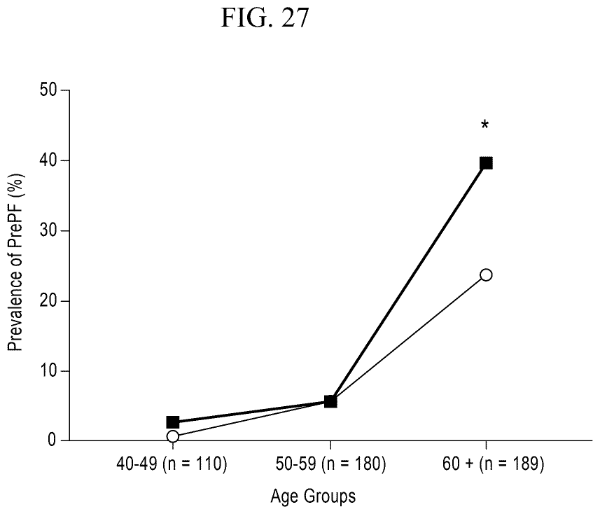

The MUC5B promoter polymorphism rs35705950 was associated with the visual diagnosis of PrePF (present in 40% of those without fibrosis versus 53% with PrePF; MAF 0 29 versus 0.21, respectively, p=0.03, FIG. 22). After age 60, there was a statistically significant difference in the proportion of subjects with visually diagnosed PrePF when the cohort was stratified by MUC5B genotype (23.8% versus 39.8% prevalence, p=0.02) (FIG. 27).

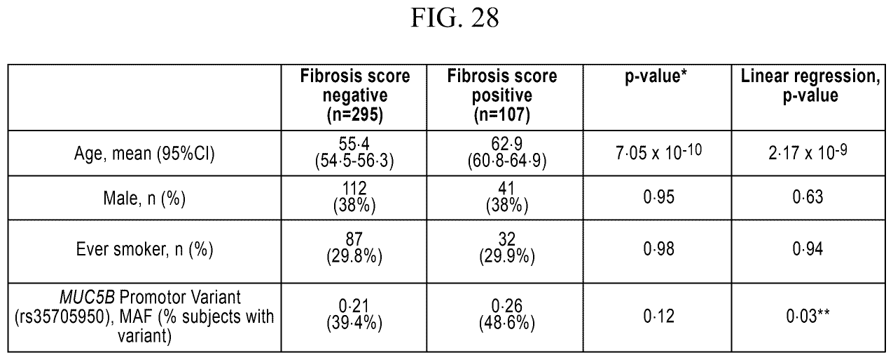

MUC5B variant carriers, regardless of their visual CT diagnosis, had significantly higher qHRCT fibrosis scores (1.3 [95% CI 1.2-1.5] versus 1.1 [95% CI 1.0-1.2], p=0.02). The association between MUC5B genotype and fibrosis score was significant even when controlling for age and male sex in linear regression (p=0.03, FIG. 28). Age was significantly associated with fibrosis score (p=2.17×10⁻⁹), but male sex (p=0.63) and smoking (p=0.94) were not. To determine whether individual textural components were driving the association of the composite fibrosis score with genotype, each score component was tested individually for association with the MUC5B variant, controlling for age and sex. Quantitative scores for reticulation, honeycombing, and ground glass were significantly associated with the MUC5B variant (p=0.02, p=0.02, p=0 04, respectively), while “not normal high density” was not (p=0.18). The simpler quantitative scoring method, log % HAA, was not significantly different in MUC5B variant carriers (p=0.4).

In contrast to the MUC5B variant, the common IPF-associated TERT polymorphism (rs2736100) was not significantly associated with PrePF assessed either qualitatively (MAF 0.47 in PrePF versus 0.46 in unaffected, p=0.77) or quantitatively (MAF 0.50 fibrotic versus 0 47 not fibrotic, p=0.40).

When these factors were examined individually for their contributions to risk of PrePF in our study cohort, we used a mixed effects logistic regression model to test the independent effects of age sex, smoking, and MUC5B or TERT genotypes while controlling for family. Age and the MUC5B genotype remained statistically significantly associated with PrePF (OR 1.15, 95% CI 1.09-1.22, p=7.34×10⁻⁷ and OR 2.18, 95% CI 1.00-4.73, p=0.05, respectively) (FIG. 22). The common TERT polymorphism (rs2736100) associated with fibrotic idiopathic interstitial pneumonia (29) was not significantly associated with PrePF (MAF was 0.45 in PrePF versus 0.45 in unaffected, p=0.88) or in a log-additive model controlling for age, sex, and smoking history (p=0.57).

Given the presence of non-fibrotic ILD (n=18, FIG. 18) in the “No Fibrosis” cohort, secondary analyses were performed that (1) excluded non-fibrotic ILDs and (2) compared all ILD (inclusive of non-fibrotic ILD) to those without any ILD. When non-fibrotic ILDs were excluded from analyses, PrePF subjects were older (p=4.7×10⁻¹³), more commonly male (p=0.04), more often had a smoking history (p=0.003), and had a higher prevalence of the MUC5B promoter variant (MAF 0.29 versus 0.20, p=0.02). However, when controlling for family relatedness and the other risk factors in a mixed effects logistic regression, only age and the MUC5B promoter variant were significantly associated with PrePF with odds ratios 1.15 (95% CI 1.09-1.22, p=9.5×10⁻⁷) and 2.16 (95% CI 1.00-4.75, p=0.05), respectively. Another secondary analysis of the data was performed in which all subjects with CT findings of ILD (fibrotic or non-fibrotic) were compared to those without any evidence of ILD. Those with CT evidence of ILD were older (mean age 64.3 years, 95% CI 62.2-66.3) compared to those without any evidence of ILD (mean age 55.7 years, 95% CI 54.8-56.6, p=4.1×10⁻¹²), more likely to be male (p=0.01), more likely to have smoked (p=0.0003), and more likely to carry the MUC5B promoter variant (MAF 0.21 versus 0.30, p=0.006). When controlling for family relatedness in a mixed effects logistic regression model, age (OR 1.10, 95% CI 1.07-1.14, p=1.21×10⁻⁹), smoking history (OR 1.72, 95% CI 1.00-2.99, p=0.04), and the MUC5B promoter variant (OR 1.73, 95% CI 1.08-2.76, p=0.02) were significantly associated with risk of ILD.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A method of treating a fibrotic lung disease in a subject comprising administering to the subject an effective amount of a therapeutic agent, wherein the subject is asymptomatic and wherein the subject is at risk of developing the fibrotic lung disease.

2. The method of claim 1, wherein the subject presents radiographic Usual Interstitial Pneumonia (UIP).

3. The method of claim 1 or 2, wherein the subject has fibrotic interstitial lung disease (FILD).

4. The method of claim 1 or 2, wherein the subject has an interstitial lung disease (ILD).

5. The method of claim 4, where in the subject has rheumatoid arthritis-associated interstitial lung disease (RA-ILD).

6. The method of any one of claims 1-3, wherein the subject has a blood relative with familial interstitial pneumonia (FIP).

7. The method of claim 6, wherein the blood relative is a sibling.

8. The method of any one of claims 1-7, wherein the subject has a mutation in a sequence encoding Mucin 5B (MUC5B), Telomerase RNA Component (TERC), Family with sequence similarity 13 member A (FAM13A), Telomerase Reverse Transcriptase (TERT), Desmoplakin (DSP), Zinc-alpha 2-Glycoprotein 1 (AZGP1), Oligonucleotide/oligosaccharide-binding Fold Containing 1 (OBFC1), ATPase Phospholipid Transporting 11A (ATP11A), Isovaleryl-CoA dehydrogenase (IVD)/Dispatched RND Transporter Family Member 2 (DISP2), Dipeptidyl Peptidase 9 (DPP9), Sialic Acid Binding Ig-Like Lectin 14 (SIGLEC14), Adrenomedullin 2 (ADM2), Tetraspanin 5 (TSPAN5), Calcium/Calmodulin-Dependent Protein Kinase Kinase 1 (CAMKK1), zinc finger with KRAB and SCAN domains 1 (ZKSCAN1), isovaleryl-CoA dehydrogenase (IVD), ATPase phospholipid transporting 11A (AK025511) or Matrix Metalloprotease-7 (MMP-7).

9. The method of claim 8, wherein the mutation comprises a polymorphism in a sequence encoding a MUC5B promoter.

10. The method of claim 9, wherein the polymorphism is rs35705950

11. The method of claim 8, wherein the mutation comprises a polymorphism in a sequence encoding a TERC 3′ untranslated region (UTR).

12. The method of claim 11, wherein the polymorphism is rs2293607.

13. The method of claim 8, wherein the mutation comprises a polymorphism in a sequence encoding intronic FAM13A.

14. The method of claim 13, wherein the polymorphism is rs2609260.

15. The method of claim 8, wherein the mutation comprises a polymorphism in a sequence encoding intronic TERT.

16. The method of claim 15, wherein the polymorphism is rs4449583.

17. The method of claim 8, wherein the mutation comprises a polymorphism in a sequence encoding intronic DSP.

18. The method of claim 17, wherein the polymorphism is rs2076295.

19. The method of claim 8, wherein the mutation comprises a polymorphism in a sequence encoding intronic ZKSCAN1.

20. The method of claim 19, wherein the polymorphism is rs6963345.

21. The method of claim 8, wherein the mutation comprises a polymorphism in a sequence encoding intronic OBFC1.

22. The method of claim 21, wherein the polymorphism is rs2488000.

23. The method of claim 8, wherein the mutation comprises a polymorphism in a sequence encoding an AK025511 3′ UTR.

24. The method of claim 23, wherein the polymorphism is rs1278769.

25. The method of claim 8, wherein the mutation comprises a polymorphism in a sequence encoding IVD.

26. The method of claim 25, wherein the polymorphism is rs35700143.

27. The method of claim 8, wherein the mutation comprises a polymorphism in a sequence encoding intronic DPP9.

28. The method of claim 27, wherein the polymorphism is rs12610495.

29. The method of any one of claims 1-28, wherein the fibrotic lung disease is pulmonary fibrosis, idiopathic pulmonary fibrosis (IPF), an interstitial lung abnormality (ILA), an asymptomatic ILA, interstitial lung disease (ILD) or rheumatoid arthritis-associated interstitial lung disease (RA-ILD).

30. The method of any one of claims 1-28, wherein the fibrotic lung disease is ILD or RA-ILD.

31. The method of any one of claims 1-28, wherein the fibrotic lung disease is pulmonary fibrosis or IPF.

32. The method of any one of claims 1-28, wherein the fibrotic lung disease is IPF.

33. The method of any one of claims 1-32, wherein the therapeutic agent comprises a N-acetylcysteine, pirfenidone, and nintedanib.

34. The method of any one of claims 1-32, wherein the therapeutic agent comprises pirfenidone.

35. The method of claim 34, wherein the effective dosage is about 2400 mg/day.

36. The method of claim 35, wherein the effective dosage is administered orally as a capsule or a tablet.

37. The method of claim 35 or 36, wherein the effective dosage is administered three times per day.

38. The method of any one of claims 35-37, wherein the effective dosage is administered according to an escalating dosage regimen.

39. The method of claim 39, wherein the escalating dosage regimen comprises
(a) administering to the subject about 800 mg of pirfenidone per day for a first week;
(b) administering to the subject about 1600 mg of pirfenidone per day for a second week; and
(c) administering to the subject about 2400 mg of pirfenidone per day for the remainder of the treatment.

40. The method of claim 38, wherein the escalating dosage regimen comprises
(a) administering to the subject a capsule or tablet comprising about 250 mg of pirfenidone three times a day for a first week;
(b) administering to the subject two capsules or tablets comprising about 250 mg of pirfenidone three times a day for a second week; and
(c) administering to the subject three capsules or tablets comprising about 250 mg of pirfenidone three times a day for the remainder of the treatment.

41. The method of claim 40, wherein the capsule or tablet comprises 267 mg of pirfenidone.

42. The method of any one of claims 1-32, wherein the therapeutic agent comprises nintedanib.

43. The method of claim 42, wherein the effective dosage is administered orally as a capsule or a tablet.

44. The method of claim 43, wherein the effective dosage is about 300 mg/day.

45. The method of claim 42 or 43, wherein the effective dosage is about 150 mg administered twice per day, wherein the daily doses are administered about 12 hours apart from one another.

46. The method of claim 43, wherein the effective dosage is about 200 mg/day.

47. The method of claim 43 or 46, wherein the effective dosage is about 100 mg administered twice per day, wherein the daily doses are administered about 12 hours apart from one another.

48. The method of any one of claims 42-47, wherein the effective dosage is administered according to a modified or interrupted dosage regimen.

49. The method of claim 48, wherein the modified or interrupted dosage regimen comprises
(a) administering to the subject about 300 mg of nintedanib per day until the subject presents an elevated level of liver enzymes compared to a control level of liver enzymes;
(b) administering to the subject about 200 mg of nintedanib per day until the subject presents the control level of liver enzymes; and
(c) administering to the subject about 300 mg of nintedanib per day for the remainder of the treatment;
wherein the control level of liver enzymes is a level detected in the subject prior to an initiation of the treatment.

50. The method of claim 48, wherein the modified or interrupted regimen comprises
(a) administering to the subject a capsule or tablet comprising about 150 mg of nintedanib twice per day until the subject presents an elevated level of liver enzymes compared to a control level of liver enzymes;
(b) administering to the subject two capsules or tablets comprising about 100 mg twice per day until the subject presents an elevated level of liver enzymes compared to a control level of liver enzymes; and
(c) administering to the subject a capsule or tablet comprising about 150 mg of nintedanib twice per day for the remainder of the treatment;
wherein the control level of liver enzymes is a level detected in the subject prior to an initiation of the treatment.

51. The method of any one of claims 1-50, wherein the therapeutic agent prevents the onset or development of a sign or symptom of the fibrotic lung disease.

52. The method of any one of claims 1-50, wherein the therapeutic agent delays the onset or development of a sign or symptom of the fibrotic lung disease when compared to the expected onset of the sign or symptom in the absence of treatment with the therapeutic agent.

53. The method of any one of claims 1-50, wherein the therapeutic agent reduces the severity of a sign or symptom of the fibrotic lung disease when compared to the expected severity of the sign or symptom in the absence of treatment with the therapeutic agent.

54. The method of any one of claims 51-53, wherein at least one sign of the fibrotic lung disease is detectable before the subject presents a symptom of the fibrotic lung disease.

55. The method of claim 54, wherein the at least one sign comprises gradual or unintended weight loss, clubbing of the fingers or toes, rapid and shallow breathing, fibrotic lesions in one or both lungs detectable by radiography, or a cough.

56. The method of claim 54, wherein the symptom comprises shortness of breath during exercise, shortness of breath at rest, a dry and hacking cough, repeated bouts of coughing, and uncontrollable bouts of coughing.

57. The method of any one of claims 1-56, wherein the method prevents the onset of a secondary condition associated with a severe form of the fibrotic lung disease.

58. The method of claim 57, wherein secondary condition comprises a collapsed lung, an infected lung, a blood clot in a lung, lung cancer, respiratory failure, pulmonary hypertension, heart failure or death.

59. A method of identifying a therapeutic agent or target thereof for the treatment of a fibrotic lung disease, comprising administering to a non-human subject a dose of a composition that modifies transcription or translation of a sequence encoding Mucin 5B (MUC5B), Telomerase RNA Component (TERC), Family with sequence similarity 13 member A (FAM13A), Telomerase Reverse Transcriptase (TERT), Desmoplakin (DSP), Zinc-alpha 2-Glycoprotein 1 (AZGP1), Oligonucleotide/oligosaccharide-binding Fold Containing 1 (OBFC1), ATPase Phospholipid Transporting 11A (ATP11A), Isovaleryl-CoA dehydrogenase (IVD)/Dispatched RND Transporter Family Member 2 (DISP2), Dipeptidyl Peptidase 9 (DPP9), Sialic Acid Binding Ig-Like Lectin 14 (SIGLEC14), Adrenomedullin 2 (ADM2), Tetraspanin 5 (TSPAN5), Calcium/Calmodulin-Dependent Protein Kinase Kinase 1 (CAM1KK1) or Matrix Metalloprotease-7 (MMP-7), wherein the dose of the composition is tolerable to the non-human subject and wherein the dose of the composition is therapeutically effective.

60. A method of identifying a therapeutic agent or target thereof for the treatment of a fibrotic lung disease, comprising administering to a non-human subject a composition that modifies an activity of a product of a sequence encoding MUC5B, TERC, FAM13A, TERT, DSP, AZGP1, OBFC1, ATP11A, IVD/DISP2, DPP9, SIGLEC14, ADM2, TSPAN5, CAMKK1 or MMP-7, wherein the dose of the composition is tolerable to the non-human subject and wherein the dose of the composition is therapeutically effective.

61. The method of claim 59, wherein the composition that modifies transcription or translation decreases or inhibits transcription or translation.

62. The method of claim 61, wherein the composition decreases or inhibits transcription or translation of a sequence encoding a gene selected from the group consisting of Leukotriene A4 Hydrolase (LTA4H), Surfactant Protein B (SFTPB), Breast Cancer Anti-Estrogen Resistance 3 (BCAR3), C—X—C motif Chemokine Ligand 13 (CXCL13), EPH Receptor A2 (EPHA2), Serum Amyloid A1 (SAA1), Phospholipase A2 Group IIA (PLA2G2A), Insulin-Like Growth Factor Binding Protein 3 (IGFBP3), C—C Motif Chemokine Ligand 28 (CCL28), S100 Calcium Binding Protein A12 (S100A12), Thromboxane A Synthase 1 (TBXAS1), Leukocyte Cell Derived Chemotaxin 1 (LECT1), Complement C3 (C3), Gastrin Releasing Peptide (GRP), C-Reactive Protein (CRP), Vitrin (VIT), Insulin-Like Growth Factor Binding Protein 1 (IGFBP1), Family with Sequence Similarity 173 Member A (FAM173A), Natriuretic Peptide A (NPPA), Secreted Frizzled Related Protein 1 (SFRP1), Ezrin (EZR), Inter-Alpha-Trypsin Inhibitor Heavy Chain Family Member 5 (ITIH5), Pleckstrin and Sec7 Domain Containing 2 (PSD2), Galectin 3 Binding Protein (LGALS3BP), Catenin Beta 1 (CTNNB1), Chromodomain Y Like 2 (CDYL2), Matrix Metallopeptidase 7 (MMP7), Apolipoprotein B (APOB), Proline and Arginine Rich End Leucine Rich Repeat Protein (PRELP), Eukaryotic Translation Initiation Factor 1A, X-linked (EIF1AX), Mesencephalic Astrocyte Derived Neurotrophic Factor (MANF), TNF Receptor Superfamily Member 13C (TNFRSF13C), Deformed Epidermal Autoregulatory Factor 1 transcription factor (DEAF1), Tumor Protein Translationally-Controlled 1 (TPT1), Unc-5 Netrin Receptor B (UNC5B), Phosphatidylethanolamine Binding Protein 1 (PEBP1), Syntaxin 8 (STX8), Polymeric Immunoglobulin Receptor (PIGR), Adenine Phosphoribosyltransferase (APRT), Matrix Metallopeptidase 3 (MMP3), Galectin 7 (LGALS7), Bruton Tyrosine Kinase (BTK), NSFL1 Cofactor (NSFL1C), FER Tyrosine Kinase (FER), Regenerating Family Member 1 Beta (REG1B), SMAD Family Member 2 (SMAD2), Interleukin 1 Receptor Like 1 (IL1RL1), C—C Motif Chemokine Ligand 18 (CCL18), Acid Phosphatase 2 Lysosomal (ACP2), Eukaryotic Translation Initiation Factor 4E Family Member 2 (EIF4E2), Neurexin 3 (NRXN3), IGF Like Family Member 1 (IGFL1), NME/NM23 Nucleoside Diphosphate Kinase 1 (NME1), Potassium Voltage-Gated Channel Isk-Related Family Member 1-Like (KCNE1L) or Neurexophilin 2 (NXPH2).

63. The method of claim 59, wherein the composition that modifies transcription or translation increases or activates transcription or translation.

64. The method of claim 63, wherein the composition increases or activates transcription or translation of a sequence encoding a gene selected from the group consisting of Surfactant Protein D (SFTPD), Glyceraldehyde-3-Phosphate Dehydrogenase (GAPDH), Histone Cluster 1 H1 Family Member C (HIST1H1C), YTH Domain Containing 1 (YTHDC1), Plexin A1 (PLXNA1), Serine Peptidase Inhibitor Kazal Type 6 (SPINK6), LDL Receptor Related Protein Associated Protein 1 (LRPAP1), Secretoglobin Family 3A Member 1 (SCGB3A1), H2A Histone Family Member Z (H2AFZ) or Chromosome 1 Open Reading Frame 162 (C1orf162).

65. The method of claim 60, wherein the composition that modifies an activity decreases or inhibits the activity.

66. The method of claim 65, wherein the composition decreases or inhibits the activity of a sequence encoding a gene selected from Leukotriene A4 Hydrolase (LTA4H), Surfactant Protein B (SFTPB), Breast Cancer Anti-Estrogen Resistance 3 (BCAR3), C—X—C motif Chemokine Ligand 13 (CXCL13), EPH Receptor A2 (EPHA2), Serum Amyloid A1 (SAA1), Phospholipase A2 Group IIA (PLA2G2A), Insulin-Like Growth Factor Binding Protein 3 (IGFBP3), C—C Motif Chemokine Ligand 28 (CCL28), S100 Calcium Binding Protein A12 (S100A12), Thromboxane A Synthase 1 (TBXAS1), Leukocyte Cell Derived Chemotaxin 1 (LECT1), Complement C3 (C3), Gastrin Releasing Peptide (GRP), C-Reactive Protein (CRP), Vitrin (VIT), Insulin-Like Growth Factor Binding Protein 1 (IGFBP1), Family with Sequence Similarity 173 Member A (FAM173A), Natriuretic Peptide A (NPPA), Secreted Frizzled Related Protein 1 (SFRP1), Ezrin (EZR), Inter-Alpha-Trypsin Inhibitor Heavy Chain Family Member 5 (ITIH5), Pleckstrin and Sec7 Domain Containing 2 (PSD2), Galectin 3 Binding Protein (LGALS3BP), Catenin Beta 1 (CTNNB1), Chromodomain Y Like 2 (CDYL2), Matrix Metallopeptidase 7 (MMP7), Apolipoprotein B (APOB), Proline and Arginine Rich End Leucine Rich Repeat Protein (PRELP), Eukaryotic Translation Initiation Factor 1A, X-linked (EIF1AX), Mesencephalic Astrocyte Derived Neurotrophic Factor (MANF), TNF Receptor Superfamily Member 13C (TNFRSF13C), Deformed Epidermal Autoregulatory Factor 1 transcription factor (DEAF1), Tumor Protein Translationally-Controlled 1 (TPT1), Unc-5 Netrin Receptor B (UNC5B), Phosphatidylethanolamine Binding Protein 1 (PEBP1), Syntaxin 8 (STX8), Polymeric Immunoglobulin Receptor (PIGR), Adenine Phosphoribosyltransferase (APRT), Matrix Metallopeptidase 3 (MMP3), Galectin 7 (LGALS7), Bruton Tyrosine Kinase (BTK), NSFL1 Cofactor (NSFL1C), FER Tyrosine Kinase (FER), Regenerating Family Member 1 Beta (REG1B), SMAD Family Member 2 (SMAD2), Interleukin 1 Receptor Like 1 (IL1RL1), C—C Motif Chemokine Ligand 18 (CCL18), Acid Phosphatase 2 Lysosomal (ACP2), Eukaryotic Translation Initiation Factor 4E Family Member 2 (EIF4E2), Neurexin 3 (NRXN3), IGF Like Family Member 1 (IGFL1), NME/NM23 Nucleoside Diphosphate Kinase 1 (NME1), Potassium Voltage-Gated Channel Isk-Related Family Member 1-Like (KCNE1L) or Neurexophilin 2 (NXPH2).

67. The method of claim 60, wherein the composition that modifies an activity increases or activates the activity.

68. The method of claim 67, wherein the composition increases or activates the activity of a sequence encoding Surfactant Protein D (SFTPD), Glyceraldehyde-3-Phosphate Dehydrogenase (GAPDH), Histone Cluster 1 H1 Family Member C (HIST1H1C), YTH Domain Containing 1 (YTHDC1), Plexin A1 (PLXNA1), Serine Peptidase Inhibitor Kazal Type 6 (SPINK6), LDL Receptor Related Protein Associated Protein 1 (LRPAP1), Secretoglobin Family 3A Member 1 (SCGB3A1), H2A Histone Family Member Z (H2AFZ) or Chromosome 1 Open Reading Frame 162 (C1orf162).

69. The method of any one of claims 60-68, wherein the non-human subject is a mammal.

70. The method of any one of claims 60-69, wherein the mammal is genetically-modified.

71. The method of claim 70, wherein the genetically-modified mammal is a model organism for the fibrotic lung disease.

72. The method of any one of claims 60-71, wherein the fibrotic lung disease is pulmonary fibrosis, idiopathic pulmonary fibrosis (IPF), an interstitial lung abnormality (ILA), or an asymptomatic ILA.

73. The method of any one of claims 60-71, wherein the fibrotic lung disease is pulmonary fibrosis or IPF.

74. The method of any one of claims 60-71, wherein the fibrotic lung disease is IPF.

75. The method of any one of claims 60-74, wherein the non-human subject carries a mutation in a sequence encoding MUC5B.

76. The method of claim 75, wherein the mutation comprises a polymorphism in a sequence encoding a MUC5B promoter.

77. The method of claim 76, wherein the polymorphism is rs35705950.

78. The method of any one of claims 60-77, wherein the non-human subject carries a mutation in a sequence encoding TERC, FAM13A, TERT, DSP, AZGP1, OBFC1, ATP11A, IVD/DISP2, DPP9, SIGLEC14, ADM2, TSPAN5, CAMKK1 or MMP-7.

79. The method of any one of claims 60-78, wherein the composition prevents the onset or development of a sign or symptom of the fibrotic lung disease.

80. The method of any one of claims 60-78, wherein the composition delays the onset or development of a sign or symptom of the fibrotic lung disease when compared to the expected onset of the sign or symptom in the absence of treatment with the composition.

81. The method of claim 80, wherein the composition delays the onset or development of a sign or symptom of the fibrotic lung disease when compared to the expected onset of the sign or symptom when treated using a standard therapeutic intervention.

82. The method of any one of claims 60-78, wherein the composition reduces the severity of a sign or symptom of the fibrotic lung disease when compared to the expected severity of the sign or symptom in the absence of treatment with the composition.

83. The method of claim 24, wherein the composition reduces the severity of a sign or symptom of the fibrotic lung disease when compared to the expected severity of the sign or symptom when treated using a standard therapeutic intervention.

84. The method of claim 81 or 83, wherein the standard therapeutic intervention comprises a N-acetylcysteine, pirfenidone, and nintedanib.

85. The method of claim 81 or 83, wherein the standard therapeutic intervention comprises pirfenidone.

86. The method of claim 85, wherein an effective dosage of pirfenidone is about 2400 mg/day.

87. The method of claim 86, wherein the effective dosage is administered orally as a capsule or a tablet.

88. The method of claim 86 or 87, wherein the effective dosage is administered three times per day.

89. The method of any one of claims 85-88, wherein the effective dosage is administered according to an escalating dosage regimen.

90. The method of claim 85, 86 or 89, wherein the escalating dosage regimen comprises
(a) administering to the non-human subject about 800 mg of pirfenidone per day for a first week;
(b) administering to the non-human subject about 1600 mg of pirfenidone per day for a second week; and
(c) administering to the non-human subject about 2400 mg of pirfenidone per day for the remainder of the treatment.

91. The method of claim 85, 86 or 89, wherein the escalating dosage regimen comprises
(a) administering to the non-human subject a capsule or tablet comprising about 250 mg of pirfenidone three times a day for a first week;
(b) administering to the non-human subject two capsules or tablets comprising about 250 mg of pirfenidone three times a day for a second week; and
(c) administering to the non-human subject three capsules or tablets comprising about 250 mg of pirfenidone three times a day for the remainder of the treatment.

92. The method of claim 91, wherein the capsule or tablet comprises 267 mg of pirfenidone.

93. The method of claim 81 or 83, wherein the standard therapeutic intervention comprises nintedanib.

94. The method of claim 93, wherein an effective dosage of nintedanib is administered orally as a capsule or a tablet.

95. The method of claim 94, wherein the effective dosage is about 300 mg/day.

96. The method of claim 94 or 95, wherein the effective dosage is about 150 mg administered twice per day, wherein the daily doses are administered about 12 hours apart from one another.

97. The method of claim 94, wherein the effective dosage is about 200 mg/day.

98. The method of claim 94 or 95, wherein the effective dosage is about 100 mg administered twice per day, wherein the daily doses are administered about 12 hours apart from one another.

99. The method of any one of claims 79-98, wherein the non-human subject presents at least one sign of the fibrotic lung disease.

100. The method of claim 99, wherein the at least one sign comprises gradual or unintended weight loss, clubbing of the fingers or toes, rapid and shallow breathing, fibrotic lesions in one or both lungs detectable by radiography, or a cough.

101. The method of any one of claims 60-100, wherein the compound prevents the onset of a secondary condition associated with a severe form of the fibrotic lung disease.

102. The method of claim 100, wherein the compound prevents the onset for at 1 year, 2 years, 3 years, 4 years, 5 years or any whole or fractional number of years in between.

103. The method of claim 101 or 102, wherein secondary condition comprises a collapsed lung, an infected lung, a blood clot in a lung, lung cancer, respiratory failure, pulmonary hypertension, heart failure or death.

104. A composition for the treatment of a fibrotic lung disease identified by the method of any one of claims 60-103.

105. A method of treating a fibrotic lung disease in a human subject comprising administering to the subject the composition of claim 104, wherein the subject is asymptomatic and wherein the subject is at risk of developing the fibrotic lung disease.

106. The method of claim 105, wherein the human subject presents radiographic Usual Interstitial Pneumonia (UIP).

107. The method of claim 105 or 106, wherein the human subject has fibrotic interstitial lung disease (FILD).

108. The method of any one of claims 105-107, wherein the human subject has a blood relative with familial interstitial pneumonia (FIP).

109. The method of claim 108, wherein the blood relative is a sibling.

110. The method of any one of claims 105-109, wherein the human subject has a mutation in a sequence encoding MUC5B, TERC, FAM13A, TERT, DSP, AZGP1, OBFC1, ATP11A, IVD/DISP2, DPP9, SIGLEC14, ADM2, TSPAN5, CAMKK1 or MMP-7.

111. The method of claim 110, wherein the mutation comprises a polymorphism in a sequence encoding a MUC5B promoter.

112. The method of claim 111, wherein the polymorphism is rs35705950.

113. The method of any one of claims 105-112, wherein the fibrotic lung disease is pulmonary fibrosis, idiopathic pulmonary fibrosis (IPF), an interstitial lung abnormality (ILA), or an asymptomatic ILA.

114. The method of any one of claims 105-112, wherein the fibrotic lung disease is pulmonary fibrosis or IPF.

115. The method of any one of claims 105-112, wherein the fibrotic lung disease is IPF.

116. The method of any one of claims 105-112, wherein the method prevents the onset of a secondary condition associated with a severe form of the fibrotic lung disease.

117. The method of claim 116, wherein secondary condition comprises a collapsed lung, an infected lung, a blood clot in a lung, lung cancer, respiratory failure, pulmonary hypertension, heart failure or death.


SEQUENCE LISTING (61 sequences)

SEQ ID NO: 1 - DNA, 1001 bp, Homo sapiens
  source 1..1001: mol_type = genomic DNA
agaaagaagtcatgaaagtaggaaccacatttttactcatctttctgtctccagcaagcagcttactgcttttcatacacattttgcttttattactcatgatttcaaaggtgtaatggttcagccacatcaatgtaacaaacagttcacactgggctcttatagtctggcctttaaaaccttcactatttatgctttcatcttaactactttgaccctcacaggtttactcactaagaacttgagtttcaagagaaaagatgacatgtttgctgcttaaacaagcaatatctaaaagcatatttagttataaacgtcttaccaagaattgatataattttcatttaaacatttttataaatagtagtttacaagatatagtaagtacatctctaaaaatacagtgtattcatgtaccttgacataaacttgtagtagtaccttagttttattcatgttgttatattaactaccatcactttgaatacatacctgttcacbgtacagtataggtcggtttaggtttattgccttaattgcttggttttgagttagtactgtagcaaatgctatcacactttgcattccctaaaaacaggtaaattcattaaggaaacagacaaagtatataataatctcgctacataaatatttcaagatcagctatctgcattctgataaaattgtttttaaaatttaagcattccttggactttgaattgtaagttgatcaaattcaaaaatgaattgttactgtattcttctctcctggccctaaaatctatctaaaacatggcatggggagtttcttaatgtttcagtgtccatttcctgggtgtttccctctaggttttttttcctcacccctcaagcttctatgtggatcccagctagagctcatactacttatccaacacacatcattgtgcaagcactcttttatattcatactagtacttttaagtgtgtgtgcggtgggaaaaggttaccaatcacatttt

SEQ ID NO: 2 - DNA, 1001 bp, Homo sapiens
  source 1..1001: mol_type = genomic DNA
gtattcatcaactcctatttcattccctcttcctgtgctcactggaagatgacatttcccagacttccaagaatgttactgagttctggaatgtaagtagaagggataagtatcacttctgtgctgtggcggttatggacctgtgaactttgcacacgccttctatcttctttttcagtgtccatttcagagggcatgttttcagatgaaaccagtagaagatggaagcagcctgtgactagaatcactgcttagggtcttgctgcctaggaatcccactctacctgcaacagactgtgaaagaaccgagaaatacactgattttgaacatagcccatactataatggggatgtttgttacagcagttagcattaaaaaccttggctaggcattggtcataattgtagaacacagcaaatgaagggaaactggaacatagaggccagtgagaactttagggttaatgaaaaatgagggcaaccaggataatttggttcttkgccaaataggaaggtgaaaccaaaggtagactggaggtcagaaaatcagtccagcacatgtgatgttttcatttagttgcctgtatgtctgtctggtctccagctcagcctggctccttgaggtaagaggcagtggctgttcacctttgcatcccagcacctggcatacaatagatgggatgaaatgttcaaactgagcctaagcttcagggtgcttatcaaagcagggaagatacacaagaggagatgattcaggtccagggcaggtcaggtatctaaacccagtctcttaggaagctggatcctccgaaccagggagaacaagctggatatgcactggatttcccagcagtactgatctagagactctcatagagtcccttttattccttggcctagggttacaactgcttatagcatctggaaagactcaacacctcaaaagagactttcagtagatacagcaaatacactcatggaattgataattaagcttcaat

SEQ ID NO: 3 - DNA, 1001 bp, Homo sapiens
  source 1..1001: mol_type = genomic DNA
attgtcgttgtttgcttttgtttattgagacagtctcactctgtcacccaggctggagtgtaatggcacaatctcggctcactgcaacctctgcctcctcggttcaagcagttctcattcctcaacctcatgagtagctgggattacaggcgcccaccaccacgcctggctaatttttgtatttttagtagagataggctttcaccatgttggccaggctggtctcaaactcctgacctcaagtgatctgcccgccttggcctcccacagtgctgggattacaggtgcaagccaccgtgcccggcataccttgatcttttaaaatgaagtctgaaacattgctacccttgtcctgagcaataagacccttagtgtattttagctctggccaccccccagcctgtgtgctgttttccctgctgacttagttctatctcaggcatcttgacacccccacaagctaagcattattaatattgttttccgtgttgagtgtttctktagctttgcccccgccctgcttttcctcctttgttccccgtctgtcttctgtctcaggcccgccgtctggggtccccttccttgtcctttgcgtggttcttctgtcttgttattgctggtaaaccccagctttacctgtgctggcctccatggcatctagcgacgtccggggacctctgcttatgatgcacagatgaagatgtggagactcacgaggagggcggtcatcttggcccgtgagtgtctggagcaccacgtggccagcgttccttagccagtgagtgacagcaacgtccgctcggcctgggttcagcctggaaaaccccaggcatgtcggggtctggtggctccgcggtgtcgagtttgaaatcgcgcaaacctgcggtgtggcgccagctctgacggtgctgcctggcgggggagtgtctgcttcctcccttctgcttgggaaccaggacaaaggatgaggctccgagccgttgtcgcccaacaggagcatg

SEQ ID NO: 4 - DNA, 1001 bp, Homo sapiens
  source 1..1001: mol_type = genomic DNA
atttgggaacctttaaaaaatattctggcttcaaaaatactccatatttacatctttggttctatctgaagtaaagccgtgatggtgtgcgtaagtgaaacaggtgcaaaggggcaacaacaaagggcgcctctctttgtctttgtgtcgcaggcggagatggacatggtggcctggggtgtggacctggcctcagtggagcagcacattaacagccaccggggcatccacaactccatcggcgactatcgctggcagctggacaaaatcaaagccgacctggtacttgtctgtgtttcattttagagtcttcaaaatatctaccgaaggatcgtgtaattactcaatcccagggagtttcttctgaaacattgctattatttctttcccagaagactggaaatgtttagaaatcccacttcttaaatggggaagtggaatcagtagccctattagagattatgttaacacttgaagaggagttaaaccagaggctgaggktgtgcaaacactcatttgcagtttgtgaataagtctctttaggggtggcagtttgtttctgcggtaagcagaacatctttttgaataggggaaatgcaacagtcttatacagtagtttgtgtcattggtgaatcctttcctaggtggtaattaaaacattatttctactgagcaaagccatatgtcatcccgacacccgctcccatgctgaaaaaagtcagacttgaaactgggttgagaattacagcataaaatcataactgatcttaagtgcttagtttcccgcaggtctctacacttgtaaatcactaaactttttttttttttttttacctgagaccatagcttctcatcctcatttcttcttctggctttttggggcttacttttgtccacctgagcccctgaccaactttctccttcatttctctaagacctagggaatcctaaatgatgtctttaaactttaagacaattttctaacacgtgagtctttaagt

SEQ ID NO: 5 - DNA, 941 bp, Homo sapiens
  source 1..941: mol_type = genomic DNA
ttctctgtgagtctttaggaaatgaggagcatgatcttctagcagtaaaacacctgtagagaattgccttatgttttttgtttgtttatttgtttgtgtgctttggtttggtttgctttttttttttttttttttttttttttgagatggagtctcgccctgttgcccaggctggagtgtagtggcgaaatctcggctcactgcaacctccacctccctggttcaagcaattcccctgtctcagcctcccgagtagctgagattacaggtgcacaccaccacgcccggctaatttttttgtatttttagtagagatggggtttcaccatgttggccagactggtctcgaacttctgacctcaggcaatccgcctgcctcagcctcccaaagcgctgggattacaggcatgagccactgcgccccgcctccatgttaatcamtctttctgatttcaaataactcattatccccatgaccttatggatttgtttttcctcttcatccacaaaattctccagagaagtctcccttgttatctcttggctgtgctttctatctcaccagttatctttctccaaagagcttcctctgcaaagaagctttgtatatgaagaccatgtgggggctgaatcaagaccaagtttcacaacctaaaagtagttcacaaagcttccttgcctctattctctgcaaatctgtaaactcttcagctgacccaatttctctctttagccttcagagattattttattttattttatttcatttcatttcatttcattttgacagaatctagctctgtcgcccaggctggagtgcagtggcaccatctttgctcactgcaacctccccctcacaggttcaagcaactgtcctgcctcagcctcccgagtagctgggattacaggcgtgagccaccacgcccagctgattttttttt

SEQ ID NO: 6 - DNA, 1001 bp, Homo sapiens
  source 1..1001: mol_type = genomic DNA
cctctactgccgtacaccccaccactcagccttggagtgcctgtgtgcagagcagggctgaggcatggtgctgctttggtggtctaggtttgctgcagggccaggtggcctgagctccaggcaggatctctggctgcactcagccctttctgcctccccaaatgctctatatcactatttgtacactgagcagagtaaagttagagagaactgttttatagaatagggctggcccccgctcccctggcctacgtgatggtccttcctggctgccaggtacttgtttgtattagagacagacactccacagggtctgttgtggcccacagcacataggcaatcagaggcagaaagcagagctgtttggacccacagagggccggctgtctgccactgaaatgtctttccagttggttgagaagcagcaggatgctctgctggtgatgtctgaaagtcccaggattctttgggtctccaaggagatcctagcatataccactrtcgtggttttaataaagagcaaaaacactttcagatggggagaagagtggaacaaaaggtattcttcctgggttgaagtctgggggaaaggcattgagaagactgggctaatggcacaaaccaatgaagtactcaagtcacctgtgatggaggccagtcatccaatggtatcaactttgtatgtggcaacacttaataaaaatctgaacaggtcttcacttgtggacacagtagactttcttgaaaaaggacagaaaagtgagccctgtgaattttcatctcacggactgacaacaatgacttgcctttaaggacagtcactcaagatgaagatgcaacaaaacccttccagttccaagtggctgatgaaaaaaaaaaaatcttaaaagcatcacagaacaacggagaaagagatcagaagactataacagatagtttgaattttaaaactcagagaaaagcaactgaggaggaaatacactgcttagaaagaagaaact

SEQ ID NO: 7 - DNA, 601 bp, Homo sapiens
  source 1..601: mol_type = genomic DNA
tggacggcctctgaaggggtctgtggggtcctggacgggtccccattcatggcaggattaacccccctcgggttctgtgtggtccaggccgcccctttgtctccactgccccctggccagaatgagggacagtgacccacccagggctgggcctggctcagactccgtcagagccgcagggcaagttcctggcacgtccgaggtgggaggctcctctgcgctccaggaggctgtgcctggccccccttcccggcaggaaccggctgtgtccctttccttcctttatcttctgttttcagcdccttcaactgtgaagaggtgaactcttcaaacacgctgagcaaacaggcccgactcccagggccgcatccgggatgtctcaatagctgtggccttgacgtccacctcggacccctgccccggacccagcccagttcccaatgggccctctgcccggggaggtgcctagtgggagggacgagggcaaagtcggggcccccacttgtttggtgtcactgtgtgccagcggccactggcgggcgaggctgttccagggtggaggcggggagggttggaccacaggcactgagcggggacagag

SEQ ID NO: 8 - DNA, 732 bp, Homo sapiens
  source 1..732: mol_type = genomic DNA
gtcattggtcaaatgtggcctgtatctaaattccaactgttagaatcatagacatctagagcttacgtcagttttagatatttcttatgaattctcagaattcatagattctcatttttattcttagacttctcagatattccgtttttgatagtatacccttctgagtctaatatgtcctaaagtgcgaacttgtacaatttttttttttttttttttttttttttttttktgataaggagttttactctgtcacccaggctggagtgcagtgacccgatctcggctcactgcaacctctgcctcccgggttcaagtgattgtgatgtctcagtctcccaagtagctgggattacaggctcctgccaccacatgcctagctaattgttatactttagtagaaatggggcttcgccgtgttagtcaggctggtcttgtactcctgacctcagttgatctgcctaccttggcccccaaggtgctgggattacaggcatgagccaccgcgcctgacccagcttcttaaattattctgggccaccagtaatgtgaatcatgtaaattaaaatatataattaaacaaaatcatatagcgattagagataatagttgtgaaatgcttgaaaaatcataggcatttaataaatagaagccattccaattaggattcttcttgattttttttcaagaccaaaaaaatactcttttaaatatttattataatactccatg

SEQ ID NO: 9 - DNA, 1320 bp, Homo sapiens
  source 1..1320: mol_type = genomic DNA
aggctgcagttagtcatgactgcgcgctgcactccagcctgggtgacaaagtgaggccctgtctcaaaaacaataaaaaatttaaaagagctgagcatggaggccactttgggaggctgaggcaggcagatctcttaagcccaggagtctgagaccagcctgggcgacatgatgaagccccatctctacaaaaaatacaaaaaaattagctgagctttatggcaaatccctgtaatcccagttacctaggaggcccaggcaggaagatggcttgagcccaaaaggttgaggctgtagtgagctgtgatcatgaacagagtgagaccctgtttcaaaacaaaatgaaaaacaaacaaacaaaaaaaccaagaaaacaagaaaacaaaaactatacaatgatgagccaaaaagcaagatatggaagaatatatatatatatatatatatatatatagtatgagtccagctatagaaagtttgaaatcaggcaacctaaacaatattgttcagggatctatacagaggcaggaagccattgagaaaggtaaggggaggattatcaccaaattcaggatggtggctcccctggggagaatatgtcaaggaggggcacatgggcttggaatactgtcttcattgacctgcgtgttgggtacacaggagtttgttatttttcacactgcatatgtgcatgtatatactctcccatatataccatgcatttcacacaagaacacaaaggctgtgtggctctgctctgcccctttccccttccagctcccattctcgtcytcagctagcagaggagggtcagggtcttttagcacagcttccttctgtctctgagtgggtcagaggagtacggggatgagggcctcccttctgcggctgggctctggccactccagggtgggaaggcctggagaaaacagggccaggcaaagccggctggccctgctgtttctgccaatgctgggattaggccagggctctggcccacctgtcatttcactcattcagcatgaacatagccactgagcacttactgtgagccccgggtgctattgggagagttcagataagtgagagagggtctttgacctcaaagatcttacagagaggaccgtatacacaaataacagtataccagcaaaatgtgagctaagtgtcatgtgactactcatctactctttcaataaatatttgttgtgcacctattacatgccaggaactgtgctggatggtgatcatgtaaagacagtcaaatcacagtcctagctctcagattcacagcctgcctaatgctggggaaactggaat

SEQ ID NO: 10 - DNA, 1001 bp, Homo sapiens
  source 1..1001: mol_type = genomic DNA
ccagccagaaggggcgcagtttgttagttcagctcctcctgagacagaaataaagacacgaaccaaaggacatcagcacttacagggctctcaggtcacacacaggatgtccgcgcccactgcagagctgcaggtcccctccagggcagtggggagccacaagcagcgttaggcagcggctgggaccaggaccgcctgagcactcaagaacccccactgccccaagcactgctggcagcaagcccagaaaactgagcccggggagctcctctgagcggcctaagcacccctctaagctgtgctgccccaattcaagcctggctcacggcagcaaagaaaaaatgtgaccttcggagctcccaaaggggccacccataagctgagagcctgcccggaagcacttatagacccgcgtggcttgttttcattgcaaagaacaataaaaattatcttgcctctgatcaccactgatagcccaagaagcaaaaattcgatcccggdgatgagaaatgaaatgaaacatcgcgagaaacttccaggaatcttctggatgtggctagactctttagcttgagcttccagacaggccgaggcttggtgctggagcctggccctccgctgacctctcttctacccgggggcacagcccggattgcagagaggctggcgcaagagtgagggagcgagggctagcctgtgatgggctttctccacctagcaccaccctatgctgtggctcaggggagtcaagagtttacacagctgcagagatggattccaggccacttactcaagtctacctactccttccttcggccaatcagctgggtgcctctgcggcctgtgacaccaccagcaaacagctccagacctcctagcatggtctctgtcaaggctgggtggcagatctgtgatctcctttttaaatttttcattttttttaagagatggggtcttgctatattgcccaggctggtctcaaactcctgggctccagcgat

SEQ ID NO: 11 - DNA, 17916 bp, Homo sapiens
  source 1..17916: mol_type = genomic DNA
cacccggcccggctccctccctgcccgtccccgtccccccacccgtgccagcccccaggatgggtgccccgagcgcgtgccggacgctggtgttggctctggcggccatgctcgtggtgccgcaggcagagacccagggccctgtggagccgagctgggagaatgcagggcacaccatggatggcggtgccccgacgtcctcgcccacccggcgcgtgagctttgttccacccgtcactgtcttccccagcctgagccccctgaacccggcgcacaatgggcgggtgtgcagcacctggggtgacttccactacaagaccttcgacggcgacgtcttccgcttccctggcctttgcaactacgtgttctctgagcactgccgcgccgcctacgaggacttcaacgtccagctacgccgaggcctagtgggctccaggcctgtggtcacccgtgttgtcatcaaggcccaggggctggtgctggaggcgtccaacggctccgtcctcatcaatgggcagcgggaggagctgccttacagccgcactggcctcctggtggagcagagcggggactacatcaaggtcagcatccggctggtgctgacattcctgtggaacggagaggacagtgccctgctggagctggatcccaaatacgccaaccagacctgtggcctgtgtggggacttcaacggcctcccggccttcaacgagttctatgcccacaacgccaggctgaccccgctccagtttgggaacctgcagaagttggatgggcccacggagcagtgcccggacccgctgcccttgccggccggcaactgcacggacgaggagggcatctgccaccgcaccctgctggggccggcctttgcggagtgccacgcactggtggacagcactgcgtacctggccgcctgcgcccaggacctgtgccgctgccccacctgcccgtgtgccacctttgtggaatactcacgccagtgcgcccacgcggggggccagccgcggaactggaggtgccctgagctctgcccccggacctgccccctcaacatgcagcaccaggagtgtggctcaccctgcacggacacctgctccaacccccagcgcgcgcagctctgcgaggaccactgtgtggacggctgcttctgccccccaggcacggtgctggatgacatcacgcactctggctgcctgcccctcgggcagtgcccctgcacccacggcggccgcacctacagcccgggcacctccttcaacaccacctgcagctcctgcacctgctccggggggctatggcagtgccaggacctgccgtgccctggcacctgctctgtgcagggcggggcccacatctccacctatgatgagaaactctacgacctgcatggtgactgcagctacgttctgtccaagaaatgtgccgacagcagcttcaccgtgctggctgagctgcggaagtgcggcctgacggacaacgagaactgcctgaaagcggtgacgctcagcctggacggcggggacacggccatccgggtccaagcggacggcggcgtgttcctcaactccatctacacgcagctgcccctgtcggcagccaacatcaccctgttcacaccctcgagcttcttcatcgtggtgcagacaggcctggggctgcagctgctggtgcagctggtgccactcatgcaggtgtttgtcaggctggaccccgcccaccagggccagatgtgcggcctgtgtgggaacttcaaccagaaccaggctgacgacttcacggccctcagcggggtggtggaggccacgggcgcagccttcgccaacacctggaaggcccaggctgcctgtgccaatgccaggaacagctttgaggacccctgctccctcagtgtggagaatgagaactacgcccggcactggtgctcgcgcctgaccgatcccaacagtgccttctcgcgctgccactccatcatcaaccccaagcccttccactcgaactgcatgtttgacacctgcaactgtgagcggagcgaggactgcctgtgcgccgcgctgtcctcctatgtgcacgcctgtgccgccaagggcgtacagctcagcgactggagggacggcgtctgcaccaagtacatgcagaactgccccaagtcccagcgctacgcctacgtggtggatgcctgccagcccacttgccgcggcctgagtgaggccgacgtcacctgcagcgtttccttcgtgcctgtggacggctgcacctgccccgcgggcaccttcctcaatgacgcgggcgcctgtgtgcccgcccaggagtgcccctgctacgctcacggcaccgtgctggctcctggagaggtggtgcacgacgagggcgccgtgtgttcatgtacgggtgggaagctaagctgcctgggagcctctctgcagaaaagcacagggtgtgcagcccccatggtgtacctggactgcagcaacagctcggcgggcacccctggggccgagtgcctccggagctgccacacgctggacgtgggctgtttcagcacacactgcgtgtccggctgtgtctgtcccccggggctggtgtcggatgggagtgggggctgcattgccgaggaggactgcccctgtgtgcacaacgaggccacctacaagcctggagagaccatcagggtcgactgcaacacctgcacctgcaggaaccggaggtgggagtgcagccaccggctctgcctgggcacctgcgtggcctacggggatggccacttcatcacctttgatggcgatcgctacagctttgaaggcagctgcgagtacatcttggcccaggactactgtggggacaacaccacccacgggaccttccgcatcgtcaccgagaacatcccctgtgggaccaccggcaccacctgctccaaggccatcaagctcttcgtggagagctacgagctgatcctccaagaggggacctttaaggcggtggcgagagggccgggtggggacccaccctacaagatacgctacatggggatcttcctggtcatcgagacccacgggatggccgtgtcctgggaccggaagaccagcgtgttcatccgactgcaccaggactacaagggcagggtctgcggcctgtgcgggaacttcgacgacaatgccatcaatgactttgccacgcgtagccggtccgtggtgggggacgcactggagtttgggaacagctggaagctctccccctcctgcccggacgccctggcacccaaggacccctgcacggccaaccccttccgcaagtcctgggcccagaagcagtgcagcatcctccacggccccaccttcgccgcctgccgctcccaggttgactccaccaagtactacgaggcctgcgtgaacgacgcgtgtgcctgcgactcgggtggcgactgcgagtgtttctgcacggctgtggctgcctacgcccaggcctgccacgacgcgggcctgtgtgtgtcctggcggactccggacacctgccccttgttctgtgacttctacaacccacatgggggctgtgagtggcactaccagccctgcggggcaccctgcctaaaaacctgccggaaccccagtgggcactgcctggtggacctgcctggcctggaaggctgctacccgaagtgcccacccagccagcccttcttcaatgaggaccagatgaagtgcgtggcccagtgtggctgctacgacaaggacggaaactactatgacgtcggtgcaagggtccccacagcggagaactgccagagctgtaactgcacacccagtggcatccagtgcgctcacagccttgaggcctgcacctgcacctatgaggacaggacctacagctaccaggacgtcatctacaacaccaccgatgggcttggcgcctgcttgatcgccatctgcggaagcaacggcaccatcatcaggaaggctgtggcatgtcctggaactccagccacaacgccattcaccttcaccaccgcctgggtcccccactccacgacaagcccggccctcccggtctccaccgtgtgtgtccgcgaggtctgccgctggtccagctggtacaatgggcaccgcccagagcccggcctgggaggcggagactttgagacgtttgaaaacctgaggcagagagggtaccaggtatgccctgtgctggctgacatcgagtgccgggcggcgcagcttcccgacatgccgctggaggagctgggccagcaggtggactgtgaccgcatgcgggggctgatgtgcgccaacagccaacagagtcccccgctctgtcacgactacgagctgcgggttctctgctgcgaatacgtgccctgtggcccctccccggccccaggcaccagccctcagccctccctcagtgccagcacggagcctgctgtgcctaccccaacccagaccacagcaaccgaaaagaccaccctatgggtgaccccgagcatccggtcgacggcggccctcacctcgcagactgggtccagctcaggccccgtgacggtcaccccctcggccccaggtaccaccacctgccagccccggtgtcagtggacagagtggtttgatgaggactaccccaagtctgaacaacttggaggggacgttgagtcctacgataagatcagggccgctggagggcacttatgccagcagcctaaggacatagagtgccaggccgagagcttccccaactggaccctggcacaggtggggcagaaggtgcactgtgacgtccacttcggcctggtgtgcaggaactgggagcaggagggcgtcttcaagatgtgctacaactacaggatccgggtcctctgctgcagtgacgaccactgcaggggacgtgccacaaccccgccaccgaccacagagctggagacggccaccaccaccaccacccaggccctgttctcaacgccgcagcctacgagtagcccggggctgaccagggctcccccggccagcaccacagcagtccccaccctctcagaaggactgacatcccccagatacacaagcacccttggtacagccaccacgggaggccccacgacgcctgcaggctccacagaacccactgtcccaggggtggccacatccacccttccaacacgctcagcccttccagggacgacggggagcttgggcacatggcgcccctcacagccacccacgctggccccaacaacaatggcaacctccagagctcgcccgacaggcacagccagcaccgcttccaaagagccgctgaccacgagcctggcgccaacactcacgagcgagctgtccacctctcaggccgagaccagcacgcccaggacagagacgacaatgagccccttgactaacaccaccaccagccagggcacgacccgctgtcaaccgaagtgtgagtggacagagtggtttgacgtggacttcccaacctcaggggttgcaggcggggacatggaaacttttgaaaacatcagggctgctgggggcaagatgtgctgggcaccaaagagcatagagtgccgggcggagaactaccccgaggtaagcatcgaccaggtcgggcaggtgctgacctgcagcctggagacggggctgacctgcaagaacgaagaccagacaggcaggttcaacatgtgcttcaactacaacgtgcgtgtgctttgctgtgacgactacagccactgccccagtaccccagccaccagctccacggccacgccctcctcaactccggggacgacctggatcctcacaaagccgaccacaacagccactacgactgcgtccactggatccacggccaccccgacctccaccctgagaacagctccccctcccaaagtgctgaccaccacggccaccacacccacagtcaccagctccaaagccactccctcctccagtccagggactgcaaccgcccttccagcactgagaagcacagccaccacacccacagctaccagcgttacacccatcccctcttcctccctgggcaccacctggacccgcctatcacagaccaccacacccacggccaccatgtccacagccacaccctcctccactccagagactgcccacacctccacagtgcttaccgccacggccaccacaactggggccaccggctctgtggccaccccctcctccaccccaggaacagctcacactaccaaagtgccaactaccacaaccacgggcttcacagccaccccctcctccagcccagggacggcactcacgcctccagtgtggatcagcacaaccaccacacccacaaccagaggctccacggtgaccccctcctccatcccggggaccacccacaccgccacagtgctgaccaccaccaccacaactgtggccactggttctatggcaacaccctcctctagcacacagaccagtggtactcccccatcactgaccaccacggccactacgatcacggccaccggctccaccaccaacccctcctcaactcctgggacaactcccatccccccagtgctgaccaccaccgccaccacacctgcagccaccagcaacacagtgactccctcctctgccctagggaccacccacacacccccagtgccgaacaccatggccaccacacacgggcgatccctgccccccagcagtccccacacggtgcgcacagcctggacttcggccacctcgggcatcttgggcaccacccacatcacagagccttccacggtgacttcccacaccctagcagcaaccaccggtaccacccagcactcgactccagccctttccagccctcaccctagcagcagaaccaccgagtcacccccttctccagggacgaccaccccgggccacaccacggccacctccaggaccacagccacggccacacccagcaagacccgcacctcgaccctgctgcccagcagccccacatcggcccccataaccacggtggtgaccatgggctgtgagccccagtgtgcctggtcagagtggctggactacagctaccccatgccggggccctctggcggggactttgacacctactccaacatccgtgcggccggaggggccgtctgtgagcagcccctgggcctcgagtgccgtgcccaggcccagcctggtgtccccctgcgggagttgggccaggtcgtggaatgcagcctggactttggcctggtctgcaggaaccgtgagcaggtggggaagttcaagatgtgcttcaactatgaaatccgtgtgttctgctgcaactacggccactgccccagcaccccggccaccagctctacggccatgccctcctccactccggggacgacctggatcctcacagagctgaccacaacagccactacgactgagtccactggatccacggccaccccgtcctccaccccagggaccacctggatcctcacagagccgagcactacagccaccgtgacggtgcccaccggatccacggccaccgcctcctccacccaggcaactgctggcaccccacatgtgagcaccacggccacgacacccacagtcaccagctccaaagccactcccttctccagtccagggactgcaaccgcccttccagcactgagaagcacagccaccacacccacagctaccagctttacagccatcccctcctcctccctgggcaccacctggacccgcctatcacagaccaccacacccacggccaccatgtccacagccacaccctcctccactccagagactgtccacacctccacagtgcttaccaccacggccaccacaaccggggccaccggctctgtggccaccccctcctccaccccaggaacagctcacactaccaaagtgctgactaccacaaccacgggcttcacagccaccccctcctccagcccagggacggcacgcacgcttccagtgtggatcagcacaaccaccacacccacaaccagaggttccacggtgaccccctcctccatcccggggaccacccacacccccacagtgctgaccaccaccaccacaactgtggccactggttctatggcaacaccctcctctagcacacagaccagtggtactcccccatcactgaccaccacggccactacgatcacggccaccggctccaccaccaacccctcctcaactccagggacaacacctatccccccagtgctgaccaccaccgccaccacacctgcagccaccagcagcacagtgactccctcctctgccctagggaccacccacacacccccagtgccgaacaccacggccaccacacacgggcgatccctgtcccccagcagtccccacacggtgcgcacagcctggacttcggccacctcaggcaccttgggcaccacccacatcacagagccttccacggggacttcccacaccccagcagcaaccaccggtaccacccagcactcgactccagccctgtccagccctcaccctagcagcaggaccaccgagtcacccccttctccagggacgaccaccccgggccacaccagggccacctccaggaccacggccacggccacacccagcaagacccgcacctcgaccctgctgcccagcagccccacatcggccccaataaccacggtggtgaccatgggctgtgagccccagtgtgcctggtcagagtggctggactacagctaccccatgccggggccctctggcggggactttgacacctactccaacatccgtgcggccggaggggccgtctgtgagcagcccctgggcctcgagtgccgtgcccaggcccagcctggtgtccccctgcgggagttgggccaggtcgtggaatgcagcctggactttggcctggtctgcaggaaccgtgagcaggtggggaagttcaagatgtgcttcaactatgaaatccgtgtgttctgctgcaactacggccactgccccagcaccccggccaccagctctacggccacgccctcctccactccagggacgacctggatcctcacagagcagaccacagcagccactacgaccgcaaccactggatccacggccatcccgtcctccaccccgggaacagctccccctcccaaagtgctgaccagcacggccaccacacccacagccaccagttccaaagccacttcctcctccagtccaaggactgcaaccacccttccagtgctgacaagcacagccaccaaatccacagctaccagctttacacccatcccctccttcacccttgggaccaccgggaccctcccagaacagaccaccacacccatggccaccatgtccacaatccacccctcctccactccggagaccacccacacctccacagtgctgaccacgaaggccaccacgacaagggccaccagttccatgtccaccccctcctccactccggggacgacctggatcctcacagagctgaccacagcagccactacaactgcagccactggccccacggccaccccgtcctccaccccagggaccacctggatcctcacagagcccagcactacagccaccgtgacggtgcccaccggatccacggccaccgcctcctccacccgggcaactgctggcaccctcaaagtgctgaccagcacggccaccacacccacagtcatcagctccagagccactccctcctccagtccagggactgcaaccgcccttccagcactgagaagcacagccaccacacccacagctaccagcgttacagccatcccctcttcctccctgggcaccgcctggacccgcctatcacagaccaccacacccacggccaccatgtccacagccacaccctcctctactccagagactgtccacacctccacagtgcttaccaccacgaccaccacaaccagggccaccggctctgtggccaccccctcctccaccccaggaacagctcacactaccaaagtgccgactaccacaaccacgggcttcacagccaccccctcctccagcccagggacggcactcacgcctccagtgtggatcagcacaaccaccacacccacaaccagaggctccacggtgaccccctcctccatcccggggaccacccacaccgccacagtgctgaccaccaccaccacaactgtggccactggttctatggcaacaccctcctctagcacacagaccagtggtactcccccatcactgaccaccacggccactacgatcacagccaccggctccaccaccaacccctcctcaactccagggacaactcccatccccccagtgctgaccaccaccgccaccacacctgcagccaccagcagcacagtgactccctcctctgccctagggaccacccacacacccccagtgccgaacaccacggccaccacacacgggcggtccctgccccccagcagtccccacacggtgcgcacagcctggacttcggccacctcgggcatcttgggcaccacccacatcacagagccttccacggtgacttcccacaccccagcagcaaccaccagtaccacccagcactcgactccagccctgtccagccctcaccctagcagcaggaccaccgagtcacccccttctccagggacgaccaccccgggccacaccaggggcacctccaggaccacagccacagccacacccagcaagacccgcacctcgaccctgctgcccagcagccccacatcggcccccataaccacggtggtgaccacgggctgtgagccccagtgtgcctggtcagagtggctggactacagctaccccatgccggggccctctggcggggactttgacacctactccaacatccgtgcggccggaggggcagtctgtgagcagcccctgggcctcgagtgccgtgcccaggcccagcctggtgtccccctgcgggagttgggccaggtcgtggaatgcagcctggactttggcctggtctgcaggaaccgtgagcaggtggggaagttcaagatgtgcttcaactatgaaatccgtgtgttctgctgcaactacggccactgccccagcaccccggccaccagctctacggccacgccctcctcaactccggggacgacctggatcctcacaaagctgaccacaacagccactacgactgagtccactggatccacggccaccccgtcctccaccccagggaccacctggatcctcacagagccgagcactacagccaccgtgacggtgcccaccggatccacggccaccgcctcctccacccaggcaactgctggcaccccacatgtgagcaccacggccacgacacccacagtcaccagctccaaagccactcccttctccagtccagggactgcaaccgcccttccagcactgagaagcacagccaccacacccacagctaccagctttacagccatcccctcctcctccctgggcaccacctggacccgcctatcacagaccaccacacccacggccaccatgtccacagccacaccctcctccactccagagactgcccacacctccacagtgcttaccaccacggccaccacaaccagggccaccggctctgtggccaccccctcttccaccccaggaacagctcacactaccaaagtgccgactaccacaaccacgggcttcacagtcaccccctcctccagcccagggacggcacgcacgcctccagtgtggatcagcacaaccaccacacccacaaccagtggctccacggtgaccccctcctccgtcccggggaccacccacacccccacagtgctgaccaccaccaccacaactgtggccactggttctatggcaacaccctcctctagcacacagaccagtggtactcccccatcactgatcaccacggccactacgatcacggccaccggctccaccaccaacccctcctcaactccagggacaacacctatccccccagtgctgaccaccaccgccaccacacctgcagccaccagcagcacagtgactccctcctctgccctagggaccacccacacacccccagtgccgaacaccacggccaccacacacgggcgatccctgtcccccagcagtccccacacggtgcgcacagcctggacttcggccacctcaggcaccttgggcaccacccacatcacagagccttccacggggacttcccacaccccagcagcaaccaccggtaccacccagcactcgactccagccctgtccagccctcaccctagcagcaggaccaccgagtcacccccttccccagggacgaccaccccgggccacaccacggccacctccaggaccacggccacggccacacccagcaagacccgcacctcgaccctgctgcccagcagccccacatcggcccccataaccacggtggtgaccacgggctgtgagccccagtgtgcctggtcagagtggctggactacagctaccccatgccggggccctctggcggggactttgacacctactccaacatccgtgcggccggaggggccgtctgtgagcagcccctgggcctcgagtgccgtgcccaggcccagcctggtgtccccctgggggagttgggccaggtcgtggaatgcagcctggactttggcctggtctgcaggaaccgtgagcaggtggggaagttcaagatgtgcttcaactatgaaatccgtgtgttctgctgcaactacggccactgccccagcaccccggccaccagctctacggccatgccctcctccactccggggacgacctggatcctcacagagctgaccacaacagccactacgactgcatccactggatccacggccaccccgtcctccaccccgggaacagctccccctcccaaagtgctgaccagcccggccaccacacccacagccaccagttccaaagccacttcctcctccagtccaaggactgcaaccacccttccagtgctgacaagcacagccaccaaatccacagctaccagcgttacacccatcccctcctccacccttgggaccaccgggaccctcccagaacagaccaccacacccgtggccaccatgtccacaatccacccctcctccactccggagaccacccacacctccacagtgctgaccacgaaggccaccacgacaagggccaccagttccacgtccaccccctcctccactccggggacgacctggatcctcacagagctgaccacagcagccactacaactgcagccactggccccacggccaccccgtcctccaccccagggaccacctggatcctcacagagctgaccacaacagccactacgactgcgtccactggatccacggccaccccgtcctccaccccagggaccacctggatcctcacagagccgagcactacagccaccgtgacggtgcccaccggatccacggccaccgcctcctccacccaggcaactgctggcaccccacatgtgagcaccacggccacgacacccacagtcaccagctccaaagccactccctcctccagtccagggactgcaactgcccttccagcactgagaagcacagccaccacacccacagctaccagctttacagccatcccctcctcctccctgggcaccacctggacccgcctatcacagaccaccacacccacggccaccatgtccacagccacaccctcctccactccagagactgtccacacctccacagtgcttaccgccacggccaccacaaccggggccaccggctctgtggccaccccctcctccaccccaggaacagctcacactaccaaagtgccgactaccacaaccacgggcttcacagccaccccctcctccagcccagggacggcactcacgcctccagtgtggatcagcacaaccaccacacccacaaccaccacacccacaaccagtggctccacggtgaccccctcctccatcccggggaccacccacaccgccagagtgctgaccaccaccaccacaactgtggccactggttctatggcaacaccctcctctagcacacagaccagtggtactcccccatcactgaccaccacggccactacgatcacggccaccggctccaccaccaacccctcctcaactccagggacaacacccatcaccccagtgctgaccagcacggccaccacacccgcagccaccagctccaaagccacttcctcctccagtccaaggactgcaaccacccttccagtgctgacaagcacagccacaaaatccacagctaccagctttacacccatcccctcctccaccctgtggaccacgtggaccgtcccagcacagaccaccacacccatgtccaccatgtccacaatccacacctcctctactccagagaccacccacacctccacagtgctgaccaccacagccaccatgacaagggccaccaattccacggccacaccctcctccactctggggacgacccggatcctcactgagctgaccacaacagccactacaactgcagccactggatccacggccaccctgtcctccaccccagggaccacctggatcctcacagagccgagcactatagccaccgtgatggtgcccaccggttccacggccaccgcctcctccactctgggaacagctcacacccccaaagtggtgaccaccatggccactatgcccacagccactgcctccacggttcccagctcgtccaccgtggggaccacccgcacccctgcagtgctccccagcagcctgccaaccttcagcgtgtccactgtgtcctcctcagtcctcaccaccctgagacccactggcttccccagctcccacttctctactccctgcttctgcagggcatttggacagtttttctcgcccggggaagtcatctacaataagaccgaccgagccggctgccatttctacgcagtgtgcaatcagcactgtgacattgaccgcttccagggcgcctgtcccacctccccaccgccagtgtcctccgccccgctgtcctcgccctcccctgcccctggctgtgacaatgccatccctctccggcaggtgaatgagacctggaccctggagaactgcacggtggccaggtgcgtgggtgacaaccgtgtcgtcctgctggacccaaagcctgtggccaacgtcacctgcgtgaacaagcacctgcccatcaaagtgtcggacccgagccagccctgtgacttccactatgagtgcgagtgcatctgcagcatgtggggcggctcccactattccacctttgacggcacctcttacaccttccggggcaactgcacctatgtcctcatgagagagatccatgcacgctttgggaatctcagcctctacctggacaaccactactgcacggcctctgccactgccgctgccgcccgctgcccccgcgccctcagcatccactacaagtccatggatatcgtcctcactgtcaccatggtgcatgggaaggaggagggcctgatcctgtttgaccaaattccggtgagcagcggtttcagcaagaacggcgtgcttgtgtctgtgctggggaccaccaccatgcgtgtggacattcctgccctgggcgtgagcgtcaccttcaatggccaagtcttccaggcccggctgccctacagcctcttccacaacaacaccgagggccagtgcggcacctgcaccaacaaccagagggacgactgtctccagcgggacggaaccactgccgccagttgcaaggacatggccaagacgtggctggtccccgacagcagaaaggatggctgctgggccccgactggcacaccccccactgccagccccgcagccccggtgtctagcacacccacccccaccccatgcccaccacagccgctctgtgatctgatgctgagccaggtctttgctgagtgccacaaccttgtgcccccgggcccattcttcaacgcctgcatcagcgaccactgcaggggccgccttgaggtgccctgccagagcctggaggcttacgcagagctctgccgcgcccggggagtgtgcagtgactggcgaggtgcaaccggtggcctgtgcgacctcacctgcccacccaccaaagtgtacaagccatgcggccccatacagcctgccacctgcaactctaggaaccagagcccacagctggaggggatggcggagggctgcttctgccctgaggaccagatcctcttcaacgcacacatgggcatctgcgtgcaggcctgcccctgcgtgggacccgatgggtttcctaaatttcccggggagcggtgggtcagcaactgccagtcctgcgtgtgtgacgagggttcagtgtcggtgcagtgcaagcccctgccctgtgacgcccagggtcagcccccgccgtgcaaccgtcccggcttcgtaaccgtgaccaggccccgggccgagaacccctgctgccccgagacggtgtgcgtgtgcaacacaaccacctgcccccagagcctgcctgtgtgcccgccagggcaggagtccatctgcacccaggaggagggcgactgctgtcccaccttccgctgcagacctcagctgtgttcgtacaatggcaccttctacggggttggtgcaaccttcccaggcgcccttccctgccacatgtgtacctgcctctctggggacacccaggacccaacggtgcaatgtcaggaggatgcctgcaacaatactacctgtccccagggctttgagtacaagagagtggccgggcagtgctgtggggagtgcgtccagaccgcctgcctcacgcccgatggccagccagtccagctgaatgaaacctgggtcaacagccatgtggacaactgcaccgtgtacctctgtgaggctgagggtggagtccatttgctgaccccacagcctgcatcctgcccagatgtgtccagctgcagggggagcctcaggaaaaccggctgctgctactcctgtgaggaggactcctgtcaagtccgcatcaacacgaccatcctgtggcaccagggctgcgagaccgaggtcaacatcaccttctgcgagggctcctgccccggagcgtccaagtactcagcagaggcccaggccatgcagcaccagtgcacctgctgccaggagaggcgggtccacgaggagacggtgcccttgcactgtcctaacggctcagccatcctgcacacctacacccacgtggatgagtgtggctgcacgcccttctgtgtccctgcgcccatggctcccccacacacccgtggcttcccggcccaggaggccactgctgtctgagaacgttctgcctccatccccatgctctgtccacctggagccaggatgtgcattgtctgatcatgaaaaccttgggcctcctctgcggagccccccggcctgtgtgtggcaccccgcgctccgtgctcctgctgcccaccccgtgggtgaaaccggccccagaagggtgaggggccagcaggacccctttcgggagggcgccactcaggagtcctaccctgggagagcctgtggcccaccttggccttgcccctccctgatgtcactgggacgccctggaacaaactaagcatgtgcgggcctatgtgtccctgccacggccggagcgcccgcgcagcacggattccagctggccacgtccggccgctggggcagacaggctggtccaggcaaggccagctgctgccaggaagctgcgacaggcaaggcggccgcctgtccatgcctgctgcagggtaactcagggctgaggtcgcaacggccaggtcagagaggggtcagcatcccaaagccccctctgctcaacccagcccagttttgcaaataaaccctgagcattgagtacgtt

SEQ ID NO: 12 - protein, 5762 aa, Homo sapiens
MGAPSACRTLVLALAAMLVVPQAETQGPVEPSWENAGHTMDGGAPTSSPTRRVSFVPPVTVFPSLSPLNPAHNGRVCSTWGDFHYKTFDGDVFRFPGLCNYVFSEHCRAAYEDFNVQLRRGLVGSRPVVTRVVIKAQGLVLEASNGSVLINGQREELPYSRTGLLVEQSGDYIKVSIRLVLTFLWNGEDSALLELDPKYANQTCGLCGDFNGLPAFNEFYAHNARLTPLQFGNLQKLDGPTEQCPDPLPLPAGNCTDEEGICHRTLLGPAFAECHALVDSTAYLAACAQDLCRCPTCPCATFVEYSRQCAHAGGQPRNWRCPELCPRTCPLNMQHQECGSPCTDTCSNPQRAQLCEDHCVDGCFCPPGTVLDDITHSGCLPLGQCPCTHGGRTYSPGTSFNTTCSSCTCSGGLWQCQDLPCPGTCSVQGGAHISTYDEKLYDLHGDCSYVLSKKCADSSFTVLAELRKCGLTDNENCLKAVTLSLDGGDTAIRVQADGGVFLNSIYTQLPLSAANITLFTPSSFFIVVQTGLGLQLLVQLVPLMQVFVRLDPAHQGQMCGLCGNFNQNQADDFTALSGVVEATGAAFANTWKAQAACANARNSFEDPCSLSVENENYARHWCSRLTDPNSAFSRCHSIINPKPFHSNCMFDTCNCERSEDCLCAALSSYVHACAAKGVQLSDWRDGVCTKYMQNCPKSQRYAYVVDACQPTCRGLSEADVTCSVSFVPVDGCTCPAGTFLNDAGACVPAQECPCYAHGTVLAPGEVVHDEGAVCSCTGGKLSCLGASLQKSTGCAAPMVYLDCSNSSAGTPGAECLRSCHTLDVGCFSTHCVSGCVCPPGLVSDGSGGCIAEEDCPCVHNEATYKPGETIRVDCNTCTCRNRRWECSHRLCLGTCVAYGDGHFITFDGDRYSFEGSCEYILAQDYCGDNTTHGTFRIVTENIPCGTTGTTCSKAIKLFVESYELILQEGTFKAVARGPGGDPPYKIRYMGIFLVIETHGMAVSWDRKTSVFIRLHQDYKGRVCGLCGNFDDNAINDFATRSRSVVGDALEFGNSWKLSPSCPDALAPKDPCTANPFRKSWAQKQCSILHGPTFAACRSQVDSTKYYEACVNDACACDSGGDCECFCTAVAAYAQACHDAGLCVSWRTPDTCPLFCDFYNPHGGCEWHYQPCGAPCLKTCRNPSGHCLVDLPGLEGCYPKCPPSQPFFNEDQMKCVAQCGCYDKDGNYYDVGARVPTAENCQSCNCTPSGIQCAHSLEACTCTYEDRTYSYQDVIYNTTDGLGACLIAICGSNGTIIRKAVACPGTPATTPFTFTTAWVPHSTTSPALPVSTVCVREVCRWSSWYNGHRPEPGLGGGDFETFENLRQRGYQVCPVLADIECRAAQLPDMPLEELGQQVDCDRMRGLMCANSQQSPPLCHDYELRVLCCEYVPCGPSPAPGTSPQPSLSASTEPAVPTPTQTTATEKTTLWVTPSIRSTAALTSQTGSSSGPVTVTPSAPGTTTCQPRCQWTEWFDEDYPKSEQLGGDVESYDKIRAAGGHLCQQPKDIECQAESFPNWTLAQVGQKVHCDVHFGLVCRNWEQEGVFKMCYNYRIRVLCCSDDHCRGRATTPPPTTELETATTTTTQALFSTPQPTSSPGLTRAPPASTTAVPTLSEGLTSPRYTSTLGTATTGGPTTPAGSTEPTVPGVATSTLPTRSALPGTTGSLGTWRPSQPPTLAPTTMATSRARPTGTASTASKEPLTTSLAPTLTSELSTSQAETSTPRTETTMSPLTNTTTSQGTTRCQPKCEWTEWFDVDFPTSGVAGGDMETFENIRAAGGKMCWAPKSIECRAENYPEVSIDQVGQVLTCSLETGLTCKNEDQTGRFNMCFNYNVRVLCCDDYSHCPSTPATSSTATPSSTPGTTWILTKPTTTATTTASTGSTATPTSTLRTAPPPKVLTTTATTPTVTSSKATPSSSPGTATALPALRSTATTPTATSVTPIPSSSLGTTWTRLSQTTTPTATMSTATPSSTPETAHTSTVLTATATTTGATGSVATPSSTPGTAHTTKVPTTTTTGFTATPSSSPGTALTPPVWISTTTTPTTRGSTVTPSSIPGTTHTATVLTTTTTTVATGSMATPSSSTQTSGTPPSLTTTATTITATGSTTNPSSTPGTTPIPPVLTTTATTPAATSNTVTPSSALGTTHTPPVPNTMATTHGRSLPPSSPHTVRTAWTSATSGILGTTHITEPSTVTSHTLAATTGTTQHSTPALSSPHPSSRTTESPPSPGTTTPGHTTATSRTTATATPSKTRTSTLLPSSPTSAPITTVVTMGCEPQCAWSEWLDYSYPMPGPSGGDFDTYSNIRAAGGAVCEQPLGLECRAQAQPGVPLRELGQVVECSLDFGLVCRNREQVGKFKMCFNYEIRVFCCNYGHCPSTPATSSTAMPSSTPGTTWILTELTTTATTTESTGSTATPSSTPGTTWILTEPSTTATVTVPTGSTATASSTQATAGTPHVSTTATTPTVTSSKATPFSSPGTATALPALRSTATTPTATSFTAIPSSSLGTTWTRLSQTTTPTATMSTATPSSTPETVHTSTVLTTTATTTGATGSVATPSSTPGTAHTTKVLTTTTTGFTATPSSSPGTARTLPVWISTTTTPTTRGSTVTPSSIPGTTHTPTVLTTTTTTVATGSMATPSSSTQTSGTPPSLTTTATTITATGSTTNPSSTPGTTPIPPVLTTTATTPAATSSTVTPSSALGTTHTPPVPNTTATTHGRSLSPSSPHTVRTAWTSATSGTLGTTHITEPSTGTSHTPAATTGTTQHSTPALSSPHPSSRTTESPPSPGTTTPGHTRATSRTTATATPSKTRTSTLLPSSPTSAPITTVVTMGCEPQCAWSEWLDYSYPMPGPSGGDFDTYSNIRAAGGAVCEQPLGLECRAQAQPGVPLRELGQVVECSLDFGLVCRNREQVGKFKMCFNYEIRVFCCNYGHCPSTPATSSTATPSSTPGTTWILTEQTTAATTTATTGSTAIPSSTPGTAPPPKVLTSTATTPTATSSKATSSSSPRTATTLPVLTSTATKSTATSFTPIPSFTLGTTGTLPEQTTTPMATMSTIHPSSTPETTHTSTVLTTKATTTRATSSMSTPSSTPGTTWILTELTTAATTTAATGPTATPSSTPGTTWILTEPSTTATVTVPTGSTATASSTRATAGTLKVLTSTATTPTVISSRATPSSSPGTATALPALRSTATTPTATSVTAIPSSSLGTAWTRLSQTTTPTATMSTATPSSTPETVHTSTVLTTTTTTTRATGSVATPSSTPGTAHTTKVPTTTTTGFTATPSSSPGTALTPPVWISTTTTPTTRGSTVTPSSIPGTTHTATVLTTTTTTVATGSMATPSSSTQTSGTPPSLTTTATTITATGSTTNPSSTPGTTPIPPVLTTTATTPAATSSTVTPSSALGTTHTPPVPNTTATTHGRSLPPSSPHTVRTAWTSATSGILGTTHITEPSTVTSHTPAATTSTTQHSTPALSSPHPSSRTTESPPSPGTTTPGHTRGTSRTTATATPSKTRTSTLLPSSPTSAPITTVVTTGCEPQCAWSEWLDYSYPMPGPSGGDFDTYSNIRAAGGAVCEQPLGLECRAQAQPGVPLRELGQVVECSLDFGLVCRNREQVGKFKMCFNYEIRVFCCNYGHCPSTPATSSTATPSSTPGTTWILTKLTTTATTTESTGSTATPSSTPGTTWILTEPSTTATVTVPTGSTATASSTQATAGTPHVSTTATTPTVTSSKATPFSSPGTATALPALRSTATTPTATSFTAIPSSSLGTTWTRLSQTTTPTATMSTATPSSTPETAHTSTVLTTTATTTRATGSVATPSSTPGTAHTTKVPTTTTTGFTVTPSSSPGTARTPPVWISTTTTPTTSGSTVTPSSVPGTTHTPTVLTTTTTTVATGSMATPSSSTQTSGTPPSLITTATTITATGSTTNPSSTPGTTPIPPVLTTTATTPAATSSTVTPSSALGTTHTPPVPNTTATTHGRSLSPSSPHTVRTAWTSATSGTLGTTHITEPSTGTSHTPAATTGTTQHSTPALSSPHPSSRTTESPPSPGTTTPGHTTATSRTTATATPSKTRTSTLLPSSPTSAPITTVVTTGCEPQCAWSEWLDYSYPMPGPSGGDFDTYSNIRAAGGAVCEQPLGLECRAQAQPGVPLGELGQVVECSLDFGLVCRNREQVGKFKMCFNYEIRVFCCNYGHCPSTPATSSTAMPSSTPGTTWILTELTTTATTTASTGSTATPSSTPGTAPPPKVLTSPATTPTATSSKATSSSSPRTATTLPVLTSTATKSTATSVTPIPSSTLGTTGTLPEQTTTPVATMSTIHPSSTPETTHTSTVLTTKATTTRATSSTSTPSSTPGTTWILTELTTAATTTAATGPTATPSSTPGTTWILTELTTTATTTASTGSTATPSSTPGTTWILTEPSTTATVTVPTGSTATASSTQATAGTPHVSTTATTPTVTSSKATPSSSPGTATALPALRSTATTPTATSFTAIPSSSLGTTWTRLSQTTTPTATMSTATPSSTPETVHTSTVLTATATTTGATGSVATPSSTPGTAHTTKVPTTTTTGFTATPSSSPGTALTPPVWISTTTTPTTTTPTTSGSTVTPSSIPGTTHTARVLTTTTTTVATGSMATPSSSTQTSGTPPSLTTTATTITATGSTTNPSSTPGTTPITPVLTSTATTPAATSSKATSSSSPRTATTLPVLTSTATKSTATSFTPIPSSTLWTTWTVPAQTTTPMSTMSTIHTSSTPETTHTSTVLTTTATMTRATNSTATPSSTLGTTRILTELTTTATTTAATGSTATLSSTPGTTWILTEPSTIATVMVPTGSTATASSTLGTAHTPKVVTTMATMPTATASTVPSSSTVGTTRTPAVLPSSLPTFSVSTVSSSVLTTLRPTGFPSSHFSTPCFCRAFGQFFSPGEVIYNKTDRAGCHFYAVCNQHCDIDRFQGACPTSPPPVSSAPLSSPSPAPGCDNAIPLRQVNETWTLENCTVARCVGDNRVVLLDPKPVANVTCVNKHLPIKVSDPSQPCDFHYECECICSMWGGSHYSTFDGTSYTFRGNCTYVLMREIHARFGNLSLYLDNHYCTASATAAAARCPRALSIHYKSMDIVLTVTMVHGKEEGLILFDQIPVSSGFSKNGVLVSVLGTTTMRVDIPALGVSVTFNGQVFQARLPYSLFHNNTEGQCGTCTNNQRDDCLQRDGTTAASCKDMAKTWLVPDSRKDGCWAPTGTPPTASPAAPVSSTPTPTPCPPQPLCDLMLSQVFAECHNLVPPGPFFNACISDHCRGRLEVPCQSLEAYAELCRARGVCSDWRGATGGLCDLTCPPTKVYKPCGPIQPATCNSRNQSPQLEGMAEGCFCPEDQILFNAHMGICVQACPCVGPDGFPKFPGERWVSNCQSCVCDEGSVSVQCKPLPCDAQGQPPPCNRPGFVTVTRPRAENPCCPETVCVCNTTTCPQSLPVCPPGQESICTQEEGDCCPTFRCRPQLCSYNGTFYGVGATFPGALPCHMCTCLSGDTQDPTVQCQEDACNNTTCPQGFEYKRVAGQCCGECVQTACLTPDGQPVQLNETWVNSHVDNCTVYLCEAEGGVHLLTPQPASCPDVSSCRGSLRKTGCCYSCEEDSCQVRINTTILWHQGCETEVNITFCEGSCPGASKYSAEAQAMQHQCTCCQERRVHEETVPLHCPNGSAILHTYTHVDECGCTPFCVPAPMAPPHTRGFPAQEATAV

SEQ ID NO: 13 - DNA, 4018 bp, Homo sapiens
  source 1..4018: mol_type = genomic DNA
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 14 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 15 - DNA, 3829 bp, Homo sapiens
  source 1..3829: mol_type = genomic DNA
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 16 - protein, 1069 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 17 - DNA, 5030 bp, Homo sapiens
  source 1..5030: mol_type = genomic DNA
attgaggagcagaaggagtagggtgcgggggaggaggaggagcgcctttagtgctgcagcagctgctgctctgattggcccggtggttcagctgcttccctggaacaaaaggtcaaagtggactgcagtgtaaatgtagagaagcagccgataaaatagcattgcctgaagaagtttggaggctgagagcagcagtagactggccaactgcagagcaagttgtttctccagccgtgcggtgcagcctcatgcccccaacccagcttagccactgtaagaagacgttcactgtacagacgaccaaacttgccgtggaagagacagttgtgagattcccttgcaaatttacatacgagaatggcttgtgaaatcatgcctctgcaaagttcacaggaagatgaaagacctctgtcacctttctatttgagtgctcatgtaccccaagtcagcaatgtgtctgcaaccggagaactcttagaaagaaccatccgatcagctgtagaacaacatctttttgatgttaataactctggaggtcaaagttcagaggactcagaatctggaacactatcagcatcttctgccacatctgccagacagcgccgccgccagtccaaggagcaggatgaagttcgacatgggagagacaagggacttatcaacaaagaaaatactccttctgggttcaaccaccttgatgattgtattttgaatactcaggaagtcgaaaaggtacacaaaaatacttttggttgtgctggagaaaggagcaagcctaaacgtcagaaatccagtactaaactttctgagcttcatgacaatcaggacggtcttgtgaatatggaaagtctcaattccacacgatctcatgagagaactggacctgatgattttgaatggatgtctgatgaaaggaaaggaaatgaaaaagatggtggacacactcagcattttgagagccccacaatgaagatccaggagcatcccagcctatctgacaccaaacagcagagaaatcaagatgccggtgaccaggaggagagctttgtctccgaagtgccccagtcggacctgactgcattgtgtgatgaaaagaactgggaagagcctatccctgctttctcctcctggcagcgggagaacagtgactctgatgaagcccacctctcgccgcaggctgggcgcctgatccgtcagctgctggacgaagacagcgaccccatgctctctcctcggttctacgcttatgggcagagcaggcaatacctggatgacacagaagtgcctccttccccaccaaactcccattctttcatgaggcggcgaagctcctctctggggtcctatgatgatgagcaagaggacctgacacctgcccagctcacacgaaggattcagagccttaaaaagaagatccggaagtttgaagatagattcgaagaagagaagaagtacagaccttcccacagtgacaaagcagccaatccggaggttctgaaatggacaaatgaccttgccaaattccggagacaacttaaagaatcaaaactaaagatatctgaagaggacctaactcccaggatgcggcagcgaagcaacacactccccaagagttttggttcccaacttgagaaagaagatgagaagaagcaagagctggtggataaagcaataaagcccagtgttgaagccacattggaatctattcagaggaagctccaggagaagcgagcggaaagcagccgccctgaggacattaaggatatgaccaaagaccagattgctaatgagaaagtggctctgcagaaagctctgttatattatgaaagcattcatggacggccggtaacaaagaacgaacggcaggtgatgaagccactatacgacaggtaccggctggtcaaacagatcctctcccgagctaacaccatacccatcattggttccccctccagcaagcggagaagccctttgctgcagccaattatcgagggcgaaactgcttccttcttcaaggagataaaggaagaagaggaggggtcagaagacgatagcaatgtgaagccagacttcatggtcactctgaaaaccgatttcagtgcacgatgctttctggaccaattcgaagatgacgctgatggatttatttccccaatggatgataaaataccatcaaaatgcagccaggacacagggctttcaaatctccatgctgcctcaatacctgaactcctggaacacctccaggaaatgagagaagaaaagaaaaggattcgaaagaaacttcgggattttgaagacaactttttcagacagaatggaagaaatgtccagaaggaagaccgcactcctatggctgaagaatacagtgaatataagcacataaaggcgaaactgaggctcctggaggtgctcatcagcaagagagacactgattccaagtccatgtgaggggcatggccaagcacagggggctggcagctgcggtgagagtttactgtccccagagaaagtgcagctctggaaggcagccttggggctggccctgcaaagcatgcagcccttctgcctctagaccatttggcatcggctcctgtttccattgcctgccttagaaactggctggaagaagacaatgtgacctgacttaggcattttgtaattggaaagtcaagactgcagtatgtgcacatgcgcacgcgcatgcacgcacacacacacacagtagtggagctttcctaacactagcagagattaatcactacattagacaacactcatctacagagaatatacactgttcttccctggataactgagaaacaagagaccattctctgtctaactgtgataaaaacaagctcaggactttattctatagagcaaacttgctgtggagggccatgctctccttggacccagttaactgcaaacgtgcattggagccctatttgctgccgctgccattctagtgacctttccacagagctgcgccttcctcacgtgtgtgaaaggttttccccttcagccctcaggtagatggaagctgcatctgcccacgatggcagtgcagtcatcatcttcaggatgtttcttcaggacttcctcagctgacaaggaattttggtccctgcctaggaccgggtcatctgcagaggacagagagatggtaagcagctgtatgaatgctgattttaaaaccaggtcatgggagaagagcctggagattctttcctgaacactgactgcacttaccagtctgattttatcgtcaaacaccaagccaggctagcatgctcatggcaatctgtttggggctgttttgttgtggcactagccaaacataaaggggcttaagtcagcctgcatacagaggatcggggagagaaggggcctgtgttctcagcctcctgagtacttaccagagtttaatttttttaaaaaaaatctgcactaaaatccccaaactgacaggtaaatgtagccctcagagctcagcccaaggcagaatctaaatcacactattttcgagatcatgtataaaaagaaaaaaaagaagtcatgctgtgtggccaattataatttttttcaaagactttgtcacaaaactgtctatattagacattttggagggaccaggaaatgtaagacaccaaatcctccatctcttcagtgtgcctgatgtcacctcatgatttgctgttacttttttaactcctgcgccaaggacagtgggttctgtgtccacctttgtgctttgcgaggccgagcccaggcatctgctcgcctgccacggctgaccagagaaggtgcttcaggagctctgccttagacgacgtgttacagtatgaacacacagcagaggcaccctcgtatgttttgaaagttgccttctgaaagggcacagttttaaggaaaagaaaaagaatgtaaaactatactgacccgttttcagttttaaagggtcgtgagaaactggctggtccaatgggatttacagcaacattttccattgctgaagtgaggtagcagctctcttctgtcagctgaatgttaaggatggggaaaaagaatgcctttaagtttgctcttaatcgtatggaagcttgagctatgtgttggaagtgccctggttttaatccatacacaaagacggtacataatcctacaggtttaaatgtacataaaaatatagtttggaattctttgctctactgtttacattgcagattgctataatttcaaggagtgagattataaataaaatgatgcactttaggatgtttcctatttttgaaatctgaacatgaatcattcacatgaccaaaaattgtgtttttttaaaaatacatgtctagtctgtcctttaatagctctcttaaataagctatgatattaatcagatcattaccagttagcttttaaagcacatttgtttaagactatgtttttggaaaaatacgctacagaatttttttttaagctacaaataaatgagatgctactaattgttttggaatctgttgtttctgccaaaggtaaattaactaaagatttattcaggaatccccatttgaatttgtatgattcaataaaagaaaacaccaagtaagttatataaaataaattgtgtatgagatgttgtgttttcctttgtaatttccactaactaactaactaacttatattcttcatggaatggagcccagaagaaatgagaggaagcccttttcacactagatcttatttgaagaaatgtttgttagtcagtcagtcagtggtttctggctctgccgagggagatgtgttccccagcaaccatttctgcagcccagaatctcaaggcactagaggcggtgtcttaattaattggcttcacaaagacaaaatgctctggactgggatttttcctttgctgtgttgggaatatgtgtttattaattagcacatgccaacaaaataaatgtcaagagttatttcataagtgtaagtaaacttaagaattaaagagtgcagacttataattttca

SEQ ID NO: 18 - protein, 697 aa, Homo sapiens
MACEIMPLQSSQEDERPLSPFYLSAHVPQVSNVSATGELLERTIRSAVEQHLFDVNNSGGQSSEDSESGTLSASSATSARQRRRQSKEQDEVRHGRDKGLINKENTPSGFNHLDDCILNTQEVEKVHKNTFGCAGERSKPKRQKSSTKLSELHDNQDGLVNMESLNSTRSHERTGPDDFEWMSDERKGNEKDGGHTQHFESPTMKIQEHPSLSDTKQQRNQDAGDQEESFVSEVPQSDLTALCDEKNWEEPIPAFSSWQRENSDSDEAHLSPQAGRLIRQLLDEDSDPMLSPRFYAYGQSRQYLDDTEVPPSPPNSHSFMRRRSSSLGSYDDEQEDLTPAQLTRRIQSLKKKIRKFEDRFEEEKKYRPSHSDKAANPEVLKWTNDLAKFRRQLKESKLKISEEDLTPRMRQRSNTLPKSFGSQLEKEDEKKQELVDKAIKPSVEATLESIQRKLQEKRAESSRPEDIKDMTKDQIANEKVALQKALLYYESIHGRPVTKNERQVMKPLYDRYRLVKQILSRANTIPIIGSPSSKRRSPLLQPIIEGETASFFKEIKEEEEGSEDDSNVKPDFMVTLKTDFSARCFLDQFEDDADGFISPMDDKIPSKCSQDTGLSNLHAASIPELLEHLQEMREEKKRIRKKLRDFEDNFFRQNGRNVQKEDRTPMAEEYSEYKHIKAKLRLLEVLISKRDTDSKSM

SEQ ID NO: 19 - DNA, 8005 bp, Homo sapiens
  source 1..8005: mol_type = genomic DNA
aagaaaccggccaggtgtggcctaggcgcccagtgccagcggggaggagactcgctccgccgccgaccaacaccaacacccagctccgacgcagctcctctgcgcccttgccgccctccgagccacagctttcctcccgctcctgcccccggcccgtcgccgtctccgcgctcgcagcggcctcgggagggcccaggtagcgagcagcgacctcgcgagccttccgcactcccgcccggttccccggccgtccgcctatccttggccccctccgctttctccgcgccggcccgcctcgcttatgcctcggcgctgagccgctctcccgattgcccgccgacatgagctgcaacggaggctcccacccgcggatcaacactctgggccgcatgatccgcgccgagtctggcccggacctgcgctacgaggtgaccagcggcggcgggggcaccagcaggatgtactattctcggcgcggcgtgatcaccgaccagaactcggacggctactgtcaaaccggcacgatgtccaggcaccagaaccagaacaccatccaggagctgctgcagaactgctccgactgcttgatgcgagcagagctcatcgtgcagcctgaattgaagtatggagatggaatacaactgactcggagtcgagaattggatgagtgttttgcccaggccaatgaccaaatggaaatcctcgacagcttgatcagagagatgcggcagatgggccagccctgtgatgcttaccagaaaaggcttcttcagctccaagagcaaatgcgagccctttataaagccatcagtgtccctcgagtccgcagggccagctccaagggtggtggaggctacacttgtcagagtggctctggctgggatgagttcaccaaacatgtcaccagtgaatgtttggggtggatgaggcagcaaagggcggagatggacatggtggcctggggtgtggacctggcctcagtggagcagcacattaacagccaccggggcatccacaactccatcggcgactatcgctggcagctggacaaaatcaaagccgacctgcgcgagaaatctgcgatctaccagttggaggaggagtatgaaaacctgctgaaagcgtcctttgagaggatggatcacctgcgacagctgcagaacatcattcaggccacgtccagggagatcatgtggatcaatgactgcgaggaggaggagctgctgtacgactggagcgacaagaacaccaacatcgctcagaaacaggaggccttctccatacgcatgagtcaactggaagttaaagaaaaagagctcaataagctgaaacaagaaagtgaccaacttgtcctcaatcagcatccagcttcagacaaaattgaggcctatatggacactctgcagacgcagtggagttggattcttcagatcaccaagtgcattgatgttcatctgaaagaaaatgctgcctactttcagttttttgaagaggcgcagtctactgaagcatacctgaaggggctccaggactccatcaggaagaagtacccctgcgacaagaacatgcccctgcagcacctgctggaacagatcaaggagctggagaaagaacgagagaaaatccttgaatacaagcgtcaggtgcagaacttggtaaacaagtctaagaagattgtacagctgaagcctcgtaacccagactacagaagcaataaacccattattctcagagctctctgtgactacaaacaagatcagaaaatcgtgcataagggggatgagtgtatcctgaaggacaacaacgagcgcagcaagtggtacgtgacgggcccgggaggcgttgacatgcttgttccctctgtggggctgatcatccctcctccgaacccactggccgtggacctctcttgcaagattgagcagtactacgaagccatcttggctctgtggaaccagctctacatcaacatgaagagcctggtgtcctggcactactgcatgattgacatagagaagatcagggccatgacaatcgccaagctgaaaacaatgcggcaggaagattacatgaagacgatagccgaccttgagttacattaccaagagttcatcagaaatagccaaggctcagagatgtttggagatgatgacaagcggaaaatacagtctcagttcaccgatgcccagaagcattaccagaccctggtcattcagctccctggctatccccagcaccagacagtgaccacaactgaaatcactcatcatggaacctgccaagatgtcaaccataataaagtaattgaaaccaacagagaaaatgacaagcaagaaacatggatgctgatggagctgcagaagattcgcaggcagatagagcactgcgagggcaggatgactctcaaaaacctccctctagcagaccagggatcttctcaccacatcacagtgaaaattaacgagcttaagagtgtgcagaatgattcacaagcaattgctgaggttctcaaccagcttaaagatatgcttgccaacttcagaggttctgaaaagtactgctatttacagaatgaagtatttggactatttcagaaactggaaaatatcaatggtgttacagatggctacttaaatagcttatgcacagtaagggcactgctccaggctattctccaaacagaagacatgttaaaggtttatgaagccaggctcactgaggaggaaactgtctgcctggacctggataaagtggaagcttaccgctgtggactgaagaaaataaaaaatgacttgaacttgaagaagtcgttgttggccactatgaagacagaactacagaaagcccagcagatccactctcagacttcacagcagtatccactttatgatctggacttgggcaagttcggtgaaaaagtcacacagctgacagaccgctggcaaaggatagataaacagatcgactttaggttatgggacctggagaaacaaatcaagcaattgaggaattatcgtgataactatcaggctttctgcaagtggctctatgatgctaaacgccgccaggattccttagaatccatgaaatttggagattccaacacagtcatgcggtttttgaatgagcagaagaacttgcacagtgaaatatctggcaaacgagacaaatcagaggaagtacaaaaaattgctgaactttgcgccaattcaattaaggattatgagctccagctggcctcatacacctcaggactggaaactctgctgaacatacctatcaagaggaccatgattcagtccccttctggggtgattctgcaagaggctgcagatgttcatgctcggtacattgaactacttacaagatctggagactattacaggttcttaagtgagatgctgaagagtttggaagatctgaagctgaaaaataccaagatcgaagttttggaagaggagctcagactggcccgagatgccaactcggaaaactgtaataagaacaaattcctggatcagaacctgcagaaataccaggcagagtgttcccagttcaaagcgaagcttgcgagcctggaggagctgaagagacaggctgagctggatgggaagtcggctaagcaaaatctagacaagtgctacggccaaataaaagaactcaatgagaagatcacccgactgacttatgagattgaagatgaaaagagaagaagaaaatctgtggaagacagatttgaccaacagaagaatgactatgaccaactgcagaaagcaaggcaatgtgaaaaggagaaccttggttggcagaaattagagtctgagaaagccatcaaggagaaggagtacgagattgaaaggttgagggttctactgcaggaagaaggcacccggaagagagaatatgaaaatgagctggcaaaggcatctaataggattcaggaatcaaagaatcagtgtactcaggtggtacaggaaagagagagccttctggtgaaaatcaaagtcctggagcaagacaaggcaaggctgcagaggctggaggatgagctgaatcgtgcaaaatcaactctagaggcagaaaccagggtgaaacagcgcctggagtgtgagaaacagcaaattcagaatgacctgaatcagtggaagactcaatattcccgcaaggaggaggctattaggaagatagaatcggaaagagaaaagagtgagagagagaagaacagtcttaggagtgagatcgaaagactccaagcagagatcaagagaattgaagagaggtgcaggcgtaagctggaggattctaccagggagacacagtcacagttagaaacagaacgctcccgatatcagagggagattgataaactcagacagcgcccatatgggtcccatcgagagacccagactgagtgtgagtggaccgttgacacctccaagctggtgtttgatgggctgaggaagaaggtgacagcaatgcagctctatgagtgtcagctgatcgacaaaacaaccttggacaaactattgaaggggaagaagtcagtggaagaagttgcttctgaaatccagccattccttcggggtgcaggatctatcgctggagcatctgcttctcctaaggaaaaatactctttggtagaggccaagagaaagaaattaatcagcccagaatccacagtcatgcttctggaggcccaggcagctacaggtggtataattgatccccatcggaatgagaagctgactgtcgacagtgccatagctcgggacctcattgacttcgatgaccgtcagcagatatatgcagcagaaaaagctatcactggttttgatgatccattttcaggcaagacagtatctgtttcagaagccatcaagaaaaatttgattgatagagaaaccggaatgcgcctgctggaagcccagattgcttcagggggtgtagtagaccctgtgaacagtgtctttttgccaaaagatgtcgccttggcccgggggctgattgatagagatttgtatcgatccctgaatgatccccgagatagtcagaaaaactttgtggatccagtcaccaaaaagaaggtcagttacgtgcagctgaaggaacggtgcagaatcgaaccacatactggtctgctcttgctttcagtacagaagagaagcatgtccttccaaggaatcagacaacctgtgaccgtcactgagctagtagattctggtatattgagaccgtccactgtcaatgaactggaatctggtcagatttcttatgacgaggttggtgagagaattaaggacttcctccagggttcaagctgcatagcaggcatatacaatgagaccacaaaacagaagcttggcatttatgaggccatgaaaattggcttagtccgacctggtactgctctggagttgctggaagcccaagcagctactggctttatagtggatcctgttagcaacttgaggttaccagtggaggaagcctacaagagaggtctggtgggcattgagttcaaagagaagctcctgtctgcagaacgagctgtcactgggtataatgatcctgaaacaggaaacatcatctctttgttccaagccatgaataaggaactcatcgaaaagggccacggtattcgcttattagaagcacagatcgcaaccggggggatcattgacccaaaggagagccatcgtttaccagttgacatagcatataagaggggctatttcaatgaggaactcagtgagattctctcagatccaagtgatgataccaaaggattttttgaccccaacactgaagaaaatcttacctatctgcaactaaaagaaagatgcattaaggatgaggaaacagggctctgtcttctgcctctgaaagaaaagaagaaacaggtgcagacatcacaaaagaataccctcaggaagcgtagagtggtcatagttgacccagaaaccaataaagaaatgtctgttcaggaggcctacaagaagggcctaattgattatgaaaccttcaaagaactgtgtgagcaggaatgtgaatgggaagaaataaccatcacgggatcagatggctccaccagggtggtcctggtagatagaaagacaggcagtcagtatgatattcaagatgctattgacaagggccttgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccggggtcccgctccggctcccgctcgggatctcgctccggatctcgctccgggtcccgcagtgggtcccggagaggaagctttgacgccacagggaattcttcctactcttattcctactcatttagcagtagttctattgggcactagtagtcagttgggagtggttgctataccttgacttcatttatatgaatttccactttattaaataatagaaaagaaaatcccggtgcttgcagtagagtgataggacattctatgcttacagaaaatatagccatgattgaaatcaaatagtaaaggctgttctggctttttatcttcttagctcatcttaaataagcagtacacttggatgcagtgcgtctgaagtgctaatcagttgtaacaatagcacaaatcgaacttaggatttgtttcttctcttctgtgtttcgatttttgatcaattctttaattttggaagcctataatacagttttctattcttggagataaaaattaaatggatcactgatattttagtcattctgcttctcatctaaatatttccatattctgtattaggagaaaattaccctcccagcaccagcccccctctcaaacccccaacccaaaaccaagcattttggaatgagtctcctttagtttcagagtgtggattgtataacccatatactcttcgatgtacttgtttggtttggtattaatttgactgtgcatgacagcggcaatcttttctttggtcaaagttttctgtttattttgcttgtcatattcgatgtactttaaggtgtctttatgaagtttgctattctggcaataaacttttagacttttgaagtgtttgtgttttaatttaatatgtttataagcatgtataaacatttagcatatttttatcataggtctaaaaatatttgtttactaaatacctgtgaagaaataccattaaaaaactatttggttctgaattcttactagaaaaaaaa

SEQ ID NO: 20 - protein, 2272 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 21 - DNA, 1278 bp, Homo sapiens
  source 1..1278: mol_type = genomic DNA
ccattggcctgtagattcacctcccctgggcagggccccaggacccaggataatatctgtgcctcctgcccagaaccctccaagcagacacaatggtaagaatggtgcctgtcctgctgtctctgctgctgcttctgggtcctgctgtcccccaggagaaccaagatggtcgttactctctgacctatatctacactgggctgtccaagcatgttgaagacgtccccgcgtttcaggcccttggctcactcaatgacctccagttctttagatacaacagtaaagacaggaagtctcagcccatgggactctggagacaggtggaaggaatggaggattggaagcaggacagccaacttcagaaggccagggaggacatctttatggagaccctgaaagacatcgtggagtattacaacgacagtaacgggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctacgtgcagcgggccaaggcttacctggaggaggagtgccctgcgactctgcggaaatacctgaaatacagcaaaaatatcctggaccggcaagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagctaataaataatcatccctccttgcctagcataaaaaaaaaaaaaaa

SEQ ID NO: 22 - protein, 298 aa, Homo sapiens
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

SEQ ID NO: 23 - DNA, 6483 bp, Homo sapiens
  source 1..6483: mol_type = genomic DNA
aaatgcgctggcggggagaccggggttggtccctggcggggcagggggcgggctcaggccggaactccagagacgacctcagccaactgctcctgcgccgggcggggtcgtcgccgccagcggctccgagcgccggaagggccaggtctcagggctcctggagctgcaggcggcgggaggggctacaaatgcttgactcagtgatgcagaacctttcagagttagctggaagccacagccctgcctcttgatgcagcctggatccagccggtgtgaagaggagaccccttccctcttgtggggtttggatcctgtgtttctagcctttgcaaaactctacatcagggatatcctggacatgaaggagtcccgccaggtgccaggtgtatttttgtacaatggacatccaataaaacaggtagatgtcttgggaactgtcattggagtgagagaaagagatgctttctacagttatggagtggatgacagcactggagttataaactgcatctgctggaaaaagttgaatactgagtctgtatcagctgctccaagtgcagcaagagagctcagcttaacctcacaacttaagaagctacaagagaccattgagcagaaaacaaagatagagatcggggacacgatccgagtcagaggcagtatccgcacatacagagaagagcgagagattcatgccaccacttactataaagtggacgacccagtgtggaacattcaaattgcaaggatgcttgagctgcccactatctacaggaaagtttatgaccagccttttcacagctcagccctagagaaagaagaggcactaagcaatccaggcgccctggacctccccagtctcacgagtttgctgagtgaaaaagccaaagaattcctcatggagaacagagtgcagagcttttaccagcaggagctggaaatggtggagtctttgctgtcccttgccaatcagcctgtgattcacagtgcctcctccgaccaagtgaattttaagaaggacaccacttccaaggcaattcatagtatatttaagaatgctatacaactgctgcaggaaaaaggacttgttttccagaaagatgatggttttgataacctatactatgtaaccagagaagacaaagacctgcacagaaagatccaccggatcattcagcaggactgccagaaaccaaatcacatggagaagggctgtcacttcctgcacatcttggcctgtgctcgcctgagcatccgcccgggcctgagcgaggctgtgctgcagcaagttctggagctcctggaggaccagagtgacattgtcagcacaatggagcactactacacagcgttctgagcagagacacgcagaccagctgaggaggacaaagataaggtggcattcacccccaggctctgactttcagcatcatgcaggggcttatctgtctggaggcagttacctcataataaactataaaatatagtcatcttgggaatgggatttggcataaatgttgttggctcccttctgtccactatgtccttggtgtacaatgactttgatctcagccatgacacaacaagaaaaccctccctgttgagctcctggctggactgtgcgttgttcgcagagcagaatggggaggaaacagtgttggcagcttaactgatgtgtgtggttggagtctcttccatggcaaagggacaccacagggtagtgaacattcaggaactgaggggcatatggcctgatcacacagttctaagcttttcaaaacttcaggttatcagagaccttcctgtgggcctctcttgctggctaagaaccggtttaggggagtagttctccctggatgagtgcttacagtttctgtggctcagttaccagcagtggggttgagacctgggtcgatgctctttacaggcctgcccagagatgggaataaacagggatccacagcgtgactatgtgtttgtcattttccttttatttccttgggaatcgaaaggtgtcccagtacatttccctgcacttacagaggtgcatgactaaatacattgtccctcgatgcccctgaagatcacggaggcagtcagccaattgcctggcaggtggtagatgttattttcagggttgccgctgagtgtgcaggatgtgctgacaccatccagacaaagactcggtatgtgcccagacaggtgatggagtcatgcttttgctcagaatgacaaggtaaaggaaaaacatctgaggtatgttgtaggcctgttctgacagcaaaatgacaaatccagccagcaaaaataaagtgtggagaaagatttggagttaattacagtcatttcacagaaggcactgccttcgtctgctgcatttgctcttgatgtgataagctcttcgtggctcagctggagatcctttaggcctggagagttgctcctctctccgtggaaacaggacagtctttatacgcagaagtccgctgcagctcgatacgtcaggctgagagctagaaccagtagattgcctcctgtcatagacttttgtaatgatgcaaacctttgctgatttctaacagtgattatgtagtggctgccctgcatcttctctgtgtacagaagggtccctagcatagagtctgcctggaatgatgtcctgggcagttcttccttgaggtcagcagctgttccacgttgaatgcatctgattagtggggctgcccaggaaggagttcagaatcagaaggtaaaaagggcatacccttgcctatagcaactctgctcttaggggtttatctcaaggagatggctacacaagtgtgaaaggatggttgcacaaggtgttcattgctgtataatctagaattctatattggggaaaatacctatagggaaaaagttaattacggttcttgggcacaatgaaatactatgcagctatgaaaaaaatgatgaaagcagacagacagtgttgccatggcacactgtccctagtagatttagtgggaagtagatagagttatagatctgtttctatagtataacaccattatctacagctccctgtgtgtatgtatatatccgtagagagagtgtatatttctgcatggaggtctttataaatgtagcacatgtacatatatatatatatacacacacacagtcgaccactcccttctcctggaagtactttccgcgtttggctttcaggacaccaagctctctggttgctccttctcaggttcctttgttcagtgctctgcctccctgaggactcagtcccagacctcttttctatctggcttgctcactggggtgtctccagcagccacatggattataccatctacatgctgtctaacacctcagtttaaacccagaatgggcctcttccctgaactgcagacccctatattcagtttgctactgacatctccacttaggtctctaatggacatctcagatttcacaggcccaaagccaggctcccaattactcctgaccccaggcttgctcctgatagtgacatgaggcagccaaatgcctaggcagagaggggagggtcccaaatgaaaccccacgttcaagcaaagatcagcctgaaggctaaaagaccagattgctggtcctggatgaaacccaccacgcagagtgggaacttctgttcctgtttgcccaccctttcccaattgttctttctgaataacgccttaaccaatcgaatgttgccttttccagtaatacctacagcctgcccctccccccattctgagcccataaaaagacccagactcccccatattaaggggactttcctgcctttgggtagggggaccacccccacgtctcctctctgttgaaaactgtttcatcactcaataaaactcccagctttgctcactcttccactgtcagcacattctcattcttctttggtgctgggcaagaactcaaccagtgtggaagccatacttggcccaggcgggtgaagtgggcgggccgtctcctgcagcaggtagcatggtcaagcgaggcccaggtgggccgtcaccagccagaggtccctggcttgcaaagtgaccgagaaaaaaatcctgtgccactcctttggaaaatgtccctgattcaggaagaggtagctccatccagttgctcaaaccaaatccattggcttctttctttctatcatacctcacatccaatctgtctgcaagtcttttggctctaccttcagaatatctccagaatcttaactgcttcaccctcctccccggcctcctcagtcctctctgcttccgccctggcccctcttgggctgttcacagcacagcagctgttgccaccctgttaatgctcccactctcctacagccttcggtcttgccccaggtaggagcctgaggctgcacagaggtcagcacggccccgcttaccctgccctcccagcccagccgcacgggccttgcacacatgcctcggcatattcctgccttagggctggtgctcctgctatttcctcttcccaggtaaccatgtgaagtgcctccctctgccctctttccagcctttacttgagtgtcaccttctcagtgaggcctgccctcattcctctttcgctgtttgcaacccatctcctgtcccccttcccagaactccctttcctacttcgtttttcttcacagtacttgatactgcctaacacactccatggtttcttacttgccctgtttattattttcccccaatagacagaatgttccatgatggcagaattctctgttttgtttccttccatgtccccagcacctagaacagtgcctgacgcatctcctaagcaatacgaccaataagtatgtgtctggctgccttccggctgccagtgtctgcctctttcctaggggcagtggttgcgggggtgctttctcacatgtcttagtaggctgtgcaggctggaagtgctcagaagtcacacccccagggagcagcctcagccaacagcaccttggctgtaaatgccccagctccctcgccctcaggtaagcattgctgaggcacacgttccatactcttttccacagttcctccgtgggactgagcaccacccagccacccacaggagcagctaacctgataaccaccagcctcaccctccctgccttacttccccgctcccctttaccacatgctgacctcccagatgcatttcttgctttccggtctctgtctcaggattggctcctggatgaacacaaactaacactatgttcacaaatatatttgggaaatgctggatgaataattatacacatcagacagattactagaaattctcaccaaagggatgcacatgttacctctgcatggtgagatctcaggtgctttttaccccacatagctatcctttggcatttttataattagcaagtgctcactcttccactgtcagtacattctcattcttcttgggcgctggacaagaattcaaccggtgtgtaagccagactcggcccgggcagtctcaaactcctgactccttatataatttctacaaaaattataaagctatttcccactccccaccccacattcatgtaacctgaagcatgagtaaaccaagaatgaggtaggcctctgtcttctaagcaacatcagaactctaagaacatgagggactcttagaaaactctctggagctaaccacagctgggtcactgctcatgtactgaagaccagccagagggttcccctgaaaaggagggaaactgagcaaacattctccagttctcttagtgtgcacatgtttcaggaggtgtgaaccccacatgtagcttgtgtaggcaagaagacaaatagtgctactgtctggtcaaggatttgtttgaagagccatgattatgcccatatggtaagccaccagtgctccccatccctgtaagacacttctttctcattattttctcctctgatggtgtgccaggatgctggccaagagaagccaagtggaaagaaggctgttcagtgacaaggaacctaagacttagtgccaaggactgaaaccaagtaaacttgtaattttccatgatggaaacatctacactttctcattagtggcctctacagcagttgccccaaagaagcgtctcattgtttttttactacatttatgtgaagcatacaggcaaactcagaaagactgtgataaggctcgccagagatgcctgcacaggtgctgggggaaaagcaggaccatcctgaagggagatggtgtctgtggacaaagaactctgcagtggttcttatttgcatgatttctgctggtggaggctgtaaatgtgagctcaaactcccacataagtgagttttcattgtaatccagaatgtttttaaatcaccctacttctattgaacttgcactatcatctgttaacctctactgtatttattaaataaacctgaataggtaaatcacagtacagcaaaa

SEQ ID NO: 24 - protein, 368 aa, Homo sapiens
MQPGSSRCEEETPSLLWGLDPVFLAFAKLYIRDILDMKESRQVPGVFLYNGHPIKQVDVLGTVIGVRERDAFYSYGVDDSTGVINCICWKKLNTESVSAAPSAARELSLTSQLKKLQETIEQKTKIEIGDTIRVRGSIRTYREEREIHATTYYKVDDPVWNIQIARMLELPTIYRKVYDQPFHSSALEKEEALSNPGALDLPSLTSLLSEKAKEFLMENRVQSFYQQELEMVESLLSLANQPVIHSASSDQVNFKKDTTSKAIHSIFKNAIQLLQEKGLVFQKDDGFDNLYYVTREDKDLHRKIHRIIQQDCQKPNHMEKGCHFLHILACARLSIRPGLSEAVLQQVLELLEDQSDIVSTMEHYYTAF

SEQ ID NO: 25 - DNA, 8795 bp, Homo sapiens
  source 1..8795: mol_type = genomic DNA
gcggccgcactagtaccccggagcccatgggcgcgccgagccgggcgcgggggcgctgaacggcggagcgggagcggccggaggagccatggactgcagcctcgtgcggacgctcgtgcacagatactgtgcaggagaagagaattgggtggacagcaggaccatctacgtgggacacagggagccacctccgggcgcagaggcctacatcccacagagatacccagacaacaggatcgtctcgtccaagtacacattttggaactttatacccaagaatttatttgaacaattcagaagagtagccaacttttatttccttatcatatttctggtgcagttgattattgatacacccacaagtccagtgacaagcggacttccactcttctttgtcattactgtgacggctatcaaacagggttatgaagactggcttcgacataaagcagacaatgccatgaaccagtgtcctgttcatttcattcagcacggcaagctcgttcggaaacaaagtcgaaagctgcgagttggggacattgtcatggttaaggaggacgagacctttccctgcgacttgatcttcctttccagcaaccggggagatgggacgtgccacgtcaccaccgccagcttggatggagaatccagccataaaacgcattacgcggtccaggacaccaaaggcttccacacagaggaggatatcggcggacttcacgccaccatcgagtgtgagcagccccagcccgacctctacaagttcgtgggtcgcatcaacgtttacagtgacctgaatgaccccgtggtgaggcccttaggatcggaaaacctgctgcttagaggagctacactgaagaacactgagaaaatctttggtgtggctatttacacgggaatggaaaccaagatggcattaaattatcaatcaaaatctcagaagcgatctgccgtggaaaaatcgatgaatgcgttcctcattgtgtatctctgcattctgatcagcaaagccctgataaacactgtgctgaaatacatgtggcagagtgagccctttcgggatgagccgtggtataatcagaaaacggagtcggaaaggcagaggaatctgttcctcaaggcattcacggacttcctggccttcatggtcctctttaactacatcatccctgtgtccatgtacgtcacggtcgagatgcagaagttcctcggctcttacttcatcacctgggacgaagacatgtttgacgaggagactggcgaggggcctctggtgaacacgtcggacctcaatgaagagctgggacaggtggagtacatcttcacagacaagaccggcaccctcacggaaaacaacatggagttcaaggagtgctgcatcgaaggccatgtctacgtgccccacgtcatctgcaacgggcaggtcctcccagagtcgtcaggaatcgacatgattgactcgtcccccagcgtcaacgggagggagcgcgaggagctgtttttccgggccctctgtctctgccacaccgtccaggtgaaagacgatgacagcgtagacggccccaggaaatcgccggacggggggaaatcctgtgtgtacatctcatcctcgcccgacgaggtggcgctggtcgaaggtgtccagagacttggctttacctacctaaggctgaaggacaattacatggagatattaaacagggagaaccacatcgaaaggtttgaattgctggaaattttgagttttgactcagtcagaaggagaatgagtgtaattgtaaaatctgctacaggagaaatttatctgttttgcaaaggagcagattcttcgatattcccccgagtgatagaaggcaaagttgaccagatccgagccagagtggagcgtaacgcagtggaggggctccgaactttgtgtgttgcttataaaaggctgatccaagaagaatatgaaggcatttgtaagctgctgcaggctgccaaagtggcccttcaagatcgagagaaaaagttagcagaagcctatgagcaaatagagaaagatcttactctgcttggtgctacagctgttgaggaccggctgcaggagaaagctgcagacaccatcgaggccctgcagaaggccgggatcaaagtctgggttctcacgggagacaagatggagacggccgcggccacgtgctacgcctgcaagctcttccgcaggaacacgcagctgctggagctgaccaccaagaggatcgaggagcagagcctgcacgacgtcctgttcgagctgagcaagacggtcctgcgccacagcgggagcctgaccagagacaacctgtccggactttcagcagatatgcaggactacggtttaattatcgacggagctgcactgtctctgataatgaagcctcgagaagacgggagttccggcaactacagggagctcttcctggaaatctgccggagctgcagcgcggtgctctgctgccgcatggcgcccttgcagaaggctcagattgttaaattaatcaaattttcaaaagagcacccaatcacgttagcaattggcgatggtgcaaatgatgtcagcatgattctggaagcgcacgtgggcataggtgtcatcggcaaggaaggccgccaggctgccaggaacagcgactatgcaatcccaaagtttaagcatttgaagaagatgctgcttgttcacgggcatttttattacattaggatctctgagctcgtgcagtacttcttctataagaacgtctgcttcatcttccctcagtttttataccagttcttctgtgggttttcacaacagactttgtacgacaccgcgtatctgaccctctacaacatcagcttcacctccctccccatcctcctgtacagcctcatggagcagcatgttggcattgacgtgctcaagagagacccgaccctgtacagggacgtcgccaagaatgccctgctgcgctggcgcgtgttcatctactggacgctcctgggactgtttgacgcactggtgttcttctttggtgcttatttcgtgtttgaaaatacaactgtgacaagcaacgggcagatatttggaaactggacgtttggaacgctggtattcaccgtgatggtgttcacagttacactaaagcttgcattggacacacactactggacttggatcaaccattttgtcatctgggggtcgctgctgttctacgttgtcttttcgcttctctggggaggagtgatctggccgttcctcaactaccagaggatgtactacgtgttcatccagatgctgtccagcgggcccgcctggctggccatcgtgctgctggtgaccatcagcctccttcccgacgtcctcaagaaagtcctgtgccggcagctgtggccaacagcaacagagagagtccagactaagagccagtgcctttctgtcgagcagtcaaccatctttatgctttctcagacttccagcagcctgagtttctgatggaacaagagcccaggctaccagagcacctgtccctcggccgcctggtacagctcccactctcagcaggtgacactcgcggcctggaaggagaaggtgtccacggagcccccacccatcctcggcggttcccatcaccactgcagttccatcccaagtcacagctgccctaggtcccgtgtgggaatgctcgtgtgatggatggtcctaagcctgtggagactgtgcacgtgcctcttcctggcccccagcaggcaaggaggggggtcacaggccttgccctcgagcatggcaccctggccgcctggacccagcactgtggttgttgagccacaccagtggcctctgggcattcggctcaacgcaggagggacattctgctggcccaccctgcgcgctgtcatgcagaggccattcccccaggcctgtgtcttcacccacctgccatcattggcctttgctgtcactgggagagaagagccgtccagggacccatggtggcccacatgtggatgccacatgctgctgtttcctgcttgcccggccaccacccatgccctccatagggtgaggtggagccatggtggtgcgtcctttactcaacaaccctccaatccggatgctgtgggaagggccgggtcactcggataccatcatccctgcggatgcaccgccgtaccctgctcatctgggagtggtttccctgcggttacgtccaagcccgcctgccctgtgtgttggggctggctgagtttcggtctccccatcaccggccgcctcgtggagaaggcagtgccacgtgggaggacaaggccacgccggcagcttccagccctgccgcagaagtgccaggatgtccatcagccactcgccagggcacggagccgtcagtccactgttacgggagaatgttgatttcgcgggtgcgagggccgggagacagatacttggctgtgatgagcagacatcctctgtccccgtggaggggtcaacaccaaggtggtgttcgtgcaccagaacctgtctcgggctgacgggggtggcacacaggacacgggtggatcccaacaggcagcaccgcacctctgcccgcctcccgcactgcagctccgcccgccgggctctgcgtccccacgtcccctcgtcccatccccacgtcccctcatcccgtcacctcgtccccacatccccttgccccgtcacctcgtcctcatgtccccttgtcctgtcacctcgtccccacgtcccctcgtctcctcatccccacgtcctctcgtccccttgtcccgtccccacataccctcgtccccatgtccccacgcagggctctccttcgtcttaggatctgtccagcgctgctctgggtgggttagcaaccccagggctgctgtgataggaagtccctgttgttctccgtactggcatttctatttctagaaataatatttgacatagccttaatggtccttaaagaagacatttcagtgtgagattcagacttcagacgctgaaactgctgcctttcaggaaagcaccaccaacgctggaggaggagccggccctcacgcccgccccgcgccacgctgtggaacggggctccggcaagtgaaacccagagggtgtttccgaggtgctcgacagtaggtatttttggaagctcagatttcaccatttgattgtataatcttttacctataaaatatttatttgaagtagagggtaaatcagcggtaagaacagtgaacacagtggttgggataaaataaggtgacaaacatcacaccaaagatgagggtagcgagcaactggcttgagcagacagaacggggaagactccactctgtcccgaggggccagccgcaggcgtccccagggccaccctgccctgaggtccttgtgtggccgccctggcttggcagccctgcccacgctgcccccgcaaacaatggtgtgtgcgtttttacagccctttttaggaacccaatatgggcataaatgtaacacctgtagcgggggcagattctctgtatgttcagttaacaaattatttgtaatgtatttttttagaaatcttaaaattgcctttgcactgaagtattttcatagctgtttatatctcttttattcatttatttaacatactgtctaattttaaaaataggtttttaaagctttcatttttaagtttatgaaattttggccactttacatttagattctggtgagagttttgactgaatgttccaatctctgatgaatgcgaattttcagatttgattttattctctacacacacctcttcttttcttggtatttctggtggcagtgattagttgaacagcacatttaaggcacgataatttgctacactttttctttacaatttgttgcaatttcatctgctttctatgtttcattgttaattgccatccttcagccttaaaaatagaagattctcacgtgaaggtttagtaagttgggtcccagctctgcctgtgtggagatagtcaccatgtacctctgacaacaagttttagtgtgaaagtcactaaacttttacacactcccaaacgtctttttaaaaattgcttgggaaattattaaatgaatgtgcctgatgatttgaaatagacaaggggcacgagataaaaaagaaaaggatgagaagatcctcagtgaatgacgttgcagggtcttcatgcaattttccacctcgcagtagttagtatttacttgccttaaactaactttgaagcaagtaatgtcaactttgagcactttgttgagttttgaaaaatcttatttgttgctgcacaggttaataaattatcaatttgtaattcagcatgttggtcagagacacggtcactgattcacacccagtccctgccacagaccgtctcagacacgcacagtgggcctgctgcatgattcacacccagtccctgccacagaccgtctcagacacgcacagtgggcctgctgcatgattcacacccagtccctgccacagaccgtctcagacacgcacagtgggcctgctgcatgcgtgttacctggcttttggctccacgctcactcatagccatgtccacatgggggcttgcacacaggatcactcacatatgtacatgtacccaccacaaacgtgcaagctcctgcacacatgcatgcacacaaacgtgtacacaagtgtgagctcctacacgcatacacacacacacgtgtacatgcaccaaagcatgtgtgacctacagacatgcagaacatgcacgtgtacacataccacagacacgcgtgtgcatgctcctacacaatacatatgcacatatcatgaacagcgtaagttcctacacacggacgtgtgatacacacatgcatgtacaggtaagcacacatgtacaagctcctacaggcttgctctcacacacgtgtatgcacagcagagagacgtatgagcttctactgcacacatgcacacacacacgcacacgtacattcactacaaacgtgcagcctcctgcacacgtgcacattcatgtgtacaccacaaatgagttcccagacgtgtaaacacacgtgcacacatcgtacacatgtgagctcccacacgtacacacagatgcacatggacacaccccaaacacgcacaggctcctacacacatgcacacacgtgtacaccacaaacgagctcccagacatgtaaacacacgtctcccacacgtgagctcccacacgtacacatgcacatgtacgcaccacaaacacatgcgcaggctcctgcaggcgtgaatacacacatgcacacacatatacacacatgtgccacaaacaagtgcacactgtcctggtgtcctgcactgcatcctgcctccttgctgaggggcccctgtgagaggcctctggatgggcatgggaagatgggctccctggcccccagcccatgcctccctgggatgaagagtccccctcctggcagaatgtctgggctttgcagagcaggccccgggggtgaagtcgcagcttcacttacaccagctgctctgtgagcaaggcttggtgccctggacaaggcccttcccctttagggaggtccagcctcgcaagctgaaacctcccctcggctcagccctataccaggcggccacagcaggactggccacacccacgccgcacctcatccgtgcacgcgtcggagcacggccagccttccgccacgagccagctgggaagggccgcggccgcctaaagccccagtcaacccagcctgtgtctgagcagacagggcgaacaagcaggccacaccgtctcgagggaggaggccagatgcggccagcgtctccaacagggtgaccatccgctcggcttgctgagcgtttaaacaaatgtttagacaggctgtggggactcccctgagttgagccttggccaggggtccggtgctgtcgcgggaaacctccagccttgttcttcaaaccactcagctcatgtgttttgcactgactagtactgaataatacaaccactcttatttaatgttagtattatttatttgacaactcagtgtctaacagcttgatatgcaggtccttgcatcctacatttctttaggaagttacccatttgtaactttaaaaacaggaaaaatatcagttggcaaatgcaatcttttttttttttaagctaaaggtgggtgaactggaatgaaaatctttctgatgttgtgtctataagcagccttgatgggatatgttagaagtgtcatgaaagtgtgattctacttttgcagaaaaatctaaagatcaatttatatagctttattttttactttatcaaagtatacagaattttaatatgcatatattgtgtctgacttaaaattataatgtctgcgtcaccatttaaaatgtctgttcattatgtaatgtaataaaagaaggtcttcaaaaatgtatttaacatgaatggtatccatagttgtcatcatcataaatactggagtttatttttaaattattaaacatagtaggtgcattaacataaatcagtctccacacagtaacatttaactgataattcattaatcagctttgaaaaattaaattgttaattaaaccaatctaacatttcagtaaagtttattttgtatgcttctgtttttaacttttatttctgtagataaactgactggataatattatattggacttttctctagattatctaagcaggagacctgaatctgcttgcaataaagaataaaagtctgcttcagtttctttataaagaaactcacacaa

SEQ ID NO: 26 - protein, 1134 aa, Homo sapiens
MDCSLVRTLVHRYCAGEENWVDSRTIYVGHREPPPGAEAYIPQRYPDNRIVSSKYTFWNFIPKNLFEQFRRVANFYFLIIFLVQLIIDTPTSPVTSGLPLFFVITVTAIKQGYEDWLRHKADNAMNQCPVHFIQHGKLVRKQSRKLRVGDIVMVKEDETFPCDLIFLSSNRGDGTCHVTTASLDGESSHKTHYAVQDTKGFHTEEDIGGLHATIECEQPQPDLYKFVGRINVYSDLNDPVVRPLGSENLLLRGATLKNTEKIFGVAIYTGMETKMALNYQSKSQKRSAVEKSMNAFLIVYLCILISKALINTVLKYMWQSEPFRDEPWYNQKTESERQRNLFLKAFTDFLAFMVLFNYIIPVSMYVTVEMQKFLGSYFITWDEDMFDEETGEGPLVNTSDLNEELGQVEYIFTDKTGTLTENNMEFKECCIEGHVYVPHVICNGQVLPESSGIDMIDSSPSVNGREREELFFRALCLCHTVQVKDDDSVDGPRKSPDGGKSCVYISSSPDEVALVEGVQRLGFTYLRLKDNYMEILNRENHIERFELLEILSFDSVRRRMSVIVKSATGEIYLFCKGADSSIFPRVIEGKVDQIRARVERNAVEGLRTLCVAYKRLIQEEYEGICKLLQAAKVALQDREKKLAEAYEQIEKDLTLLGATAVEDRLQEKAADTIEALQKAGIKVWVLTGDKMETAAATCYACKLFRRNTQLLELTTKRIEEQSLHDVLFELSKTVLRHSGSLTRDNLSGLSADMQDYGLIIDGAALSLIMKPREDGSSGNYRELFLEICRSCSAVLCCRMAPLQKAQIVKLIKFSKEHPITLAIGDGANDVSMILEAHVGIGVIGKEGRQAARNSDYAIPKFKHLKKMLLVHGHFYYIRISELVQYFFYKNVCFIFPQFLYQFFCGFSQQTLYDTAYLTLYNISFTSLPILLYSLMEQHVGIDVLKRDPTLYRDVAKNALLRWRVFIYWTLLGLFDALVFFFGAYFVFENTTVTSNGQIFGNWTFGTLVFTVMVFTVTLKLALDTHYWTWINHFVIWGSLLFYVVFSLLWGGVIWPFLNYQRMYYVFIQMLSSGPAWLAIVLLVTISLLPDVLKKVLCRQLWPTATERVQTKSQCLSVEQSTIFMLSQTSSSLSF

SEQ ID NO: 27 - DNA, 4575 bp, Homo sapiens
  source 1..4575: mol_type = genomic DNA
tttccgcagttaggggctgctatttcaacgcagggagataaaaagaaaaaaacacttgctcttctaccccgctaaaaacactcatcctagggagcacgccagcatttgcagcgttcggggcagggccactcggcctgcggccgttgcactggctggaagctggcaggcgatcacggttgattggctcgggtgcggtccaagggcagcaacgccttcggcgggccgcctagggtgattggctgctgcagcccaccccctagccggtttggtgggcggcgaagcctggattggtggagctaagagctggctcagtttcagcgctggctcttcgtgcatggcagagatggcgactgcgactcggctgctggggtggcgtgtggcgagctggaggctgcggccgccgcttgccggcttcgtttcccagcgggcccactcgcttttgcccgtggacgatgcaatcaatgggctaagcgaggagcagaggcaggaattttggaagcagctggggaacctgggcgtattgggcatcacagcccctgttcagtatggcggctccggcctgggctacctggagcatgtgctggtgatggaggagatatcccgagcttccggagcagtggggctcagttacggtgcccactccaacctctgcatcaaccagcttgtacgcaatgggaatgaggcccagaaagagaagtatctcccgaagctgatcagtggtgagtacatcggagccctggccatgagtgagcccaatgcaggctctgatgttgtctctatgaagctcaaagcggaaaagaaaggaaatcactacatcctgaatggcaacaagttctggatcactaatggccctgatgctgacgtcctgattgtctatgccaagacagatctggctgctgtgccagcttctcggggcatcacagccttcattgtggagaagggtatgcctggctttagcacctctaagaagctggacaagctggggatgaggggctctaacacctgtgagctaatctttgaagactgcaagattcctgctgccaacatcctgggccatgagaataagggtgtctacgtgctgatgagtgggctggacctggagcggctggtgctggccggggggcctcttgggctcatgcaagcggtcctggaccacaccattccctacctgcacgtgagggaagcctttggccagaagatcggccacttccagttgatgcaggggaagatggctgacatgtacacccgcctcatggcgtgtcggcagtatgtctacaatgtcgccaaggcctgcgatgagggccattgcactgctaaggactgtgcaggtgtgattctttactcagctgagtgtgccacacaggtagccctggacggcattcagtgttttggtggcaatggctacatcaatgactttcccatgggccgctttcttcgagatgccaagctgtatgagataggggctgggaccagcgaggtgaggcggctggtcatcggcagagccttcaatgcagactttcactagtcctgagacccttcgcccccttttcctgcacctagtggcctttcttgggaagtagagatgtggcggctttcccaccctgcccacagcaggccctcctgcccagctgctcttgtcagccctctggcctctggatgaggttgagttctccacaacagctcccaagcatcatgggcctcgcagccgggcctgtgccacggctagtgttgtgtgatttaaaatggactcagcaggaagcatattgtctggggattgttgggacaggttttggtgactctgtgcccttgctctctaacttctgagcccacctcccagggtaggcacctgggggcatgcaggtgcccacctcccagggtaggcacctgggggcatgcaggtacccacctctttctcttgggtgaggctctggcaaggagatctctctgctcaagcacagcagaatcatggcccctctccatgaattggaacttggtacaggttaagtatccctaatcctgaaatctgaaacacttgtggttccaagcattttggataaggcaaattcaactttcagtctcttttctgggggaaaaaaataataaacctagcctagccaggcgtggtggctcatgcttgtaatcccagcacttcaggaggctgagatgggtggatcacctgaggtcaggagttcaagaccagcctggccaacatgtggaaacctcgcctcaactaaaaatagaaaaaaattagttgggcatggtggtgggcacctgtaatcccagctacttcaggaggctgaggcaggagaattacttgaacccaggaggcggacgttgcagtgagccgagcttgtgccattgcactccagcctgggcgacaagagcaaaactcttcaaaaaacaaaacaaaacaaaaaaaccctggcccttgtttcttccagtttctagaggtatcagctcctagcagcttatgaacacatatgcttgcttggccaggcaaggtggtgtgtgcctgtaatcccagcactttgggaggccaaggcaggtggatcacttgcagtcaggagttcaagaccagcctgtccaacgtggtgaaaccccatctctactaaaaatacaaaaattagccaggggtggtggtgcacgtctgtaatcccagctactcaggaggctgaggcaggagaatcacttgaacccgggaggtggaggttgcaatgagccaatatgacaccgctgcagtccagcctgggccatagagtgagactctgtctcaaaaaaggaaagaaaaataggctgggcacagtgactcatgcctgtaatcccaacactttgggaggccgaggcaggtggatcacgaggtcaggagttcaagaccagcctggccaagatggtaaaacctcgtctctactaaaaatacaaaaattagccaggtgtggtggcaggctcctgtaatcccagctactcaggaggctgaggcagagaattgcttgaacccgggaggcagagtttgcagtgagccaagatcacaccactgcactccagcttggacgacagagcgagactctgtctcaaaaaataataggccaggcatggtggctcaacgtctgtaatcccagcactttgggaggccgaggcgggcagatcacaaggtcaggagttcgagaccagcctgacgaccaacatggtgaaacctcgtctctactaaaaatacaaaaattagccaggcctggtggcacgcgcctgtaatcccagttacacagaagactgaggcaggagaatcgcttgaacgcaggaggcagaggttgcaggagctgagatcgcgccattgcactccagcctgggcaacagagtgagactctgtctcaaaaaataataataaaataaatgaacacacatgctgctgagtccgcagggggggcagagcagaggacagcgtgcttttgtgtactgttggaagactggctcctcctgtacagcacctctgagcccttgtgcaccgccctgccacgggcaccatccagtcctggccgtgtgaccacccacagctgactgggcagcaggcacaggccctacccgagcaggccggagttggctcgcatgactccagctgaggctgcctgtgtacatttctccagataccctatggctaattttgttataactgcacagtggctgctgccattttgtattaaatatattgtgaaacaaacctatctggggagaagcaatctacttgccgctgcttcctgtctggatccagcttgtgtccttggagagtggctggcccaggtcctattcctgtcctccagcccgttctttcatgagggacaggaaggtaaaatcagcccttaggagagaggtctcagcctccctttcccagatctcccagtgagttttaaaggaagcagggagcccagagtgctaagttcttacagccagaaggaagcttatagatttctgaaaaccgcccctttgtttttaaaaagatcaacacaatttgactttctcaaggtcaaaacgaactagaatccagatctgctcatggcaaaaatgggggtgttctgagaattccagctttgggccgcactgtacagcagtctggatagagtgtgatctgagaagggaatgggtctgggttgttccaccccttccgagttccaaaaagagggaactggttttcttggttctcagcccagcagcacctatcctggctcttggtcctggcctgcagccaagtgctgttcctagcctgaggcttgagacaggtggggttggctcctcaccaaccccagttccgtcccatcctgagggcaagatcctgggctcataggcagtccctttcacttccttgtcttgctccctgctatgttggagatgaatgtgactaaaagggccatcttgctggcttaatgtgtggctggagagaccagcctggagacaatgtggcaaaatggggcgcttcatccagtctgtctaagccctgtcgacttggggaggtgatttctttcctggttctatatgtgaagcaaaataaatgttttaaaattaaaagcaaaaaaaacaaaatgaaccatg

SEQ ID NO: 28 - protein, 396 aa, Homo sapiens
MAEMATATRLLGWRVASWRLRPPLAGFVSQRAHSLLPVDDAINGLSEEQRQEFWKQLGNLGVLGITAPVQYGGSGLGYLEHVLVMEEISRASGAVGLSYGAHSNLCINQLVRNGNEAQKEKYLPKLISGEYIGALAMSEPNAGSDVVSMKLKAEKKGNHYILNGNKFWITNGPDADVLIVYAKTDLAAVPASRGITAFIVEKGMPGFSTSKKLDKLGMRGSNTCELIFEDCKIPAANILGHENKGVYVLMSGLDLERLVLAGGPLGLMQAVLDHTIPYLHVREAFGQKIGHFQLMQGKMADMYTRLMACRQYVYNVAKACDEGHCTAKDCAGVILYSAECATQVALDGIQCFGGNGYINDFPMGRFLRDAKLYEIGAGTSEVRRLVIGRAFNADFH

SEQ ID NO: 29 - DNA, 4286 bp, Homo sapiens
  source 1..4286: mol_type = genomic DNA
caacttccgggtcaaaggtgcctgagccggcgggtcccctgtgtccgccgcggctgtcgtcccccgctcccgccacttccggggtcgcagtcccgggcatggagccgcgaccgtgaggcgccgctggacccgggacgacctgcccagtccggccgccgccccacgtcccggtctgtgtcccacgcctgcagctggaatggaggctctctggaccctttagaaggcacccctgccctcctgaggtcagctgagcggttaatgcggaaggttaagaaactgcgcctggacaaggagaacaccggaagttggagaagcttctcgctgaattccgagggggctgagaggatggccaccaccgggaccccaacggccgaccgaggcgacgcagccgccacagatgacccggccgcccgcttccaggtgcagaagcactcgtgggacgggctccggagcatcatccacggcagccgcaagtactcgggcctcattgtcaacaaggcgccccacgacttccagtttgtgcagaagacggatgagtctgggccccactcccaccgcctctactacctgggaatgccatatggcagccgagagaactccctcctctactctgagattcccaagaaggtccggaaagaggctctgctgctcctgtcctggaagcagatgctggatcatttccaggccacgccccaccatggggtctactctcgggaggaggagctgctgagggagcggaaacgcctgggggtcttcggcatcacctcctacgacttccacagcgagagtggcctcttcctcttccaggccagcaacagcctcttccactgccgcgacggcggcaagaacggcttcatggtgtcccctatgaaaccgctggaaatcaagacccagtgctcagggccccggatggaccccaaaatctgccctgccgaccctgccttcttctccttcatcaataacagcgacctgtgggtggccaacatcgagacaggcgaggagcggcggctgaccttctgccaccaaggtttatccaatgtcctggatgaccccaagtctgcgggtgtggccaccttcgtcatacaggaagagttcgaccgcttcactgggtactggtggtgccccacagcctcctgggaaggttcagagggcctcaagacgctgcgaatcctgtatgaggaagtcgatgagtccgaggtggaggtcattcacgtcccctctcctgcgctagaagaaaggaagacggactcgtatcggtaccccaggacaggcagcaagaatcccaagattgccttgaaactggctgagttccagactgacagccagggcaagatcgtctcgacccaggagaaggagctggtgcagcccttcagctcgctgttcccgaaggtggagtacatcgccagggccgggtggacccgggatggcaaatacgcctgggccatgttcctggaccggccccagcagtggctccagctcgtcctcctccccccggccctgttcatcccgagcacagagaatgaggagcagcggctagcctctgccagagctgtccccaggaatgtccagccgtatgtggtgtacgaggaggtcaccaacgtctggatcaatgttcatgacatcttctatcccttcccccaatcagagggagaggacgagctctgctttctccgcgccaatgaatgcaagaccggcttctgccatttgtacaaagtcaccgccgttttaaaatcccagggctacgattggagtgagcccttcagccccggggaagatgaatttaagtgccccattaaggaagagattgctctgaccagcggtgaatgggaggttttggcgaggcacggctccaagatctgggtcaatgaggagaccaagctggtgtacttccagggcaccaaggacacgccgctggagcaccacctctacgtggtcagctatgaggcggccggcgagatcgtacgcctcaccacgcccggcttctcccatagctgctccatgagccagaacttcgacatgttcgtcagccactacagcagcgtgagcacgccgccctgcgtgcacgtctacaagctgagcggccccgacgacgaccccctgcacaagcagccccgcttctgggctagcatgatggaggcagccagctgccccccggattatgttcctccagagatcttccatttccacacgcgctcggatgtgcggctctacggcatgatctacaagccccacgccttgcagccagggaagaagcaccccaccgtcctctttgtatatggaggcccccaggtgcagctggtgaataactccttcaaaggcatcaagtacttgcggctcaacacactggcctccctgggctacgccgtggttgtgattgacggcaggggctcctgtcagcgagggcttcggttcgaaggggccctgaaaaaccaaatgggccaggtggagatcgaggaccaggtggagggcctgcagttcgtggccgagaagtatggcttcatcgacctgagccgagttgccatccatggctggtcctacgggggcttcctctcgctcatggggctaatccacaagccccaggtgttcaaggtggccatcgcgggtgccccggtcaccgtctggatggcctacgacacagggtacactgagcgctacatggacgtccctgagaacaaccagcacggctatgaggcgggttccgtggccctgcacgtggagaagctgcccaatgagcccaaccgcttgcttatcctccacggcttcctggacgaaaacgtgcactttttccacacaaacttcctcgtctcccaactgatccgagcagggaaaccttaccagctccagatctaccccaacgagagacacagtattcgctgccccgagtcgggcgagcactatgaagtcacgttgctgcactttctacaggaatacctctgagcctgcccaccgggagccgccacatcacagcacaagtggctgcagcctccgcggggaaccaggcgggagggactgagtggcccgcgggccccagtgaggcactttgtcccgcccagcgctggccagccccgaggagccgctgccttcaccgccccgacgccttttatccttttttaaacgctcttgggttttatgtccgctgcttcttggttgccgagacagagagatggtggtctcgggccagcccctcctctccccgccttctgggaggaggaggtcacacgctgatgggcactggagaggccagaagagactcagaggagcgggctgccttccgcctggggctccctgtgacctctcagtcccctggcccggccagccaccgtccccagcacccaagcatgcaattgcctgtcccccccggccagcctccccaacttgatgtttgtgttttgtttggggggatatttttcataattatttaaaagacaggccgggcgcggtggctcacgtctgtaatcccagcactttgggaggctgaggcgggcggatcacctgaggttgggagttcaagaccagcctggccaacatggggaaaccccgtctctactaaaaatacaaaaaattagccgggtgtggtggcgcgtgcctataatcccagctactcgggaggctgaggcaggagaatcgcttgaacccgggaggtggaggttgcggtgagccaagatcgcaccattgcactccagcctgggcaacaagagcgaaactctgtctcaaaataaataaaaaataaaagacagaaagcaaggggtgcctaaatctagacttggggtccacaccgggcagcggggttgcaacccagcacctggtaggctccatttcttcccaagcccgagcagagggtcatgcgggccccacaggagaagcggccagggcccgcggggggcaccacctgtggacagccctcctgtccccaagctttcaggcaggcactgaaacgcaccgaacttccacgctctgctggtcagtggcggctgtcccctccccagcccagccgcccagccacatgtgtctgcctgacccgtacacaccaggggttccggggttgggagctgaaccatccccacctcagggttatatttccctctccccttccctccccgccaagagctctgccaggggcgggcaaaaaaaaaagtaaaaagaaaagaaaaaaaaaaaaaagaaacaaaccacctctacatattatggaaagaaaatatttttgtcgattcttattcttttataattatgcgtggaagaagtagacacattaaacgattccagttggaaaaaaaaaaaaaaaaaaa

SEQ ID NO: 30 - protein, 892 aa, Homo sapiens
MRKVKKLRLDKENTGSWRSFSLNSEGAERMATTGTPTADRGDAAATDDPAARFQVQKHSWDGLRSIIHGSRKYSGLIVNKAPHDFQFVQKTDESGPHSHRLYYLGMPYGSRENSLLYSEIPKKVRKEALLLLSWKQMLDHFQATPHHGVYSREEELLRERKRLGVFGITSYDFHSESGLFLFQASNSLFHCRDGGKNGFMVSPMKPLEIKTQCSGPRMDPKICPADPAFFSFINNSDLWVANIETGEERRLTFCHQGLSNVLDDPKSAGVATFVIQEEFDRFTGYWWCPTASWEGSEGLKTLRILYEEVDESEVEVIHVPSPALEERKTDSYRYPRTGSKNPKIALKLAEFQTDSQGKIVSTQEKELVQPFSSLFPKVEYIARAGWTRDGKYAWAMFLDRPQQWLQLVLLPPALFIPSTENEEQRLASARAVPRNVQPYVVYEEVTNVWINVHDIFYPFPQSEGEDELCFLRANECKTGFCHLYKVTAVLKSQGYDWSEPFSPGEDEFKCPIKEEIALTSGEWEVLARHGSKIWVNEETKLVYFQGTKDTPLEHHLYVVSYEAAGEIVRLTTPGFSHSCSMSQNFDMFVSHYSSVSTPPCVHVYKLSGPDDDPLHKQPRFWASMMEAASCPPDYVPPEIFHFHTRSDVRLYGMIYKPHALQPGKKHPTVLFVYGGPQVQLVNNSFKGIKYLRLNTLASLGYAVVVIDGRGSCQRGLRFEGALKNQMGQVEIEDQVEGLQFVAEKYGFIDLSRVAIHGWSYGGFLSLMGLIHKPQVFKVAIAGAPVTVWMAYDTGYTERYMDVPENNQHGYEAGSVALHVEKLPNEPNRLLILHGFLDENVHFFHTNFLVSQLIRAGKPYQLQIYPNERHSIRCPESGEHYEVTLLHFLQEYL

SEQ ID NO: 31 - DNA, 2113 bp, Homo sapiens
  source 1..2113: mol_type = genomic DNA
actcaccctccggcttcctgtcggggctttctcagccccaccccacgtttggacatttggagcatttccttccctgacagccggacctgggactgggctggggccctggcggatggagacatgctgcccctgctgctgctgcccctgctgtggggggggtccctgcaggagaagccagtgtacgagctgcaagtgcagaagtcggtgacggtgcaggagggcctgtgcgtccttgtgccctgctccttctcttacccctggagatcctggtattcctctcccccactctacgtctactggttccgggacggggagatcccatactacgctgaggttgtggccacaaacaacccagacagaagagtgaagccagagacccagggccgattccgcctccttggggatgtccagaagaagaactgctccctgagcatcggagatgccagaatggaggacacgggaagctatttcttccgcgtggagagaggaagggatgtaaaatatagctaccaacagaataagctgaacttggaggtgacagccctgatagagaaacccgacatccactttctggagcctctggagtccggccgccccacaaggctgagctgcagccttccaggatcctgtgaagcgggaccacctctcacattctcctggacggggaatgccctcagccccctggaccccgagaccacccgctcctcggagctcaccctcacccccaggcccgaggaccatggcaccaacctcacctgtcaggtgaaacgccaaggagctcaggtgaccacggagagaactgtccagctcaatgtctcctatgctccacagaacctcgccatcagcatcttcttcagaaatggcacaggcacagccctgcggatcctgagcaatggcatgtcggtgcccatccaggagggccagtccctgttcctcgcctgcacagttgacagcaacccccctgcctcactgagctggttccgggagggaaaagccctcaatccttcccagacctcaatgtctgggaccctggagctgcctaacataggagctagagagggaggggaattcacctgccgggttcagcatccgctgggctcccagcacctgtccttcatcctttctgtgcagagaagctcctcttcctgcatatgtgtaactgagaaacagcagggctcctggcccctcgtcctcaccctgatcaggggggctctcatgggggctggcttcctcctcacctatggcctcacctggatctactataccaggtgtggaggcccccagcagagcagggctgagaggcctggctgagcccctcccgctcaagacagaactgaggtgtggacacttagccctgtgggacacatgcaggacatcactgtcagcttctttctggaagctcacatcccactgactacccctcttttccttcctgccccataccccttctacttattcccctctgcttgtgagtcttgccccaccacacctgcatccccatctgcaccccatcccctctccacctgcccttctcttccctctccatccaccatctccagccctgtgaagggaatgtactttcggtcttatacccccattacccattacccaaaagttaccttttttttttttttttttttttgagacagagtctcactctgttgcacaggctggagttcagtggcacaatctccgttcactgcaacctccacctctggggttcaagcaattctcctgcctcagcctccctagtagctgggattacaggtgcctgccaccacatccagttaattttttttttttgtatgttagtagagatggggttttaccatgttggccaggtctcgaactcctgacctcaagcaatccactgcattggcctcccaaagtgctggcattacaggtatgagccaccgtgcctggctgccaaaagttaccttcttaacacttgaatttctggtctcctcagcttccctatccatataggcacagagaggcagcatttgttttccagttaaaactctacctcattgtgattattatccaatacaattgttacaaaataagtaaaacttttatgaaacaatacaacataactgattttactctttaa

SEQ ID NO: 32 - protein, 396 aa, Homo sapiens
MLPLLLLPLLWGGSLQEKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQVKRQGAQVTTERTVQLNVSYAPQNLAISIFFRNGTGTALRILSNGMSVPIQEGQSLFLACTVDSNPPASLSWFREGKALNPSQTSMSGTLELPNIGAREGGEFTCRVQHPLGSQHLSFILSVQRSSSSCICVTEKQQGSWPLVLTLIRGALMGAGFLLTYGLTWIYYTRCGGPQQSRAERPG

SEQ ID NO: 33 - DNA, 4045 bp, Homo sapiens
  source 1..4045: mol_type = genomic DNA
cgcccacgcccggcgccccgaccgcggaggactccccgagccccgcccgccatggcccggatcccgacggccgccctgggttgcatcagcctcctctgcctgcagctccctggctcgctgtcccgcagcctgggcggggacccgcgacccgtcaaacccagggagcccccagcccggagcccttccagcagcctgcagcccaggcaccccgcaccccgacctgtggtctggaagcttcaccgggccctccaggcacagaggggtgccggcctggcccctgttatgggtcagcctctccgggatggtggccgccaacactcgggcccccgaagacactcgggcccccgcaggacccaagcccagctcctgcgagtgggctgtgtgctgggcacctgccaggtgcagaatctcagccaccgcctgtggcaactcatgggaccggccggccggcaggactcagctcctgtggaccccagcagcccccacagctatggctgaggtggggccgggccacacccctgcccatcccagccagggtgctgtgcccccgtccagagctgcagctgagccccatctgaagcccagtccctcggagctgcagacagcaggtcctgcagcaacaatacctgcacggctttgcacacgtaaacctaggctggtctacacgcagtgctggtacgtcaaggagcctaaacaccctgaaattgtgaccccctgggggacagctgccagacacagctggcggcagcaccagatgctaagcgcttcagagaggaggtgtctgcccagagatgtggagcagaagctgggccctgaacacacggggccatgtctggacgagcaggggagagaggctgaactggccagaagtggcccctccgctgctggtccagtcagactgaagcccggccttgtgcctgggctgttcctgctctcatgcacaaccagcccttccacgtgcctgcctgtgggacaggagggggagcgtgggatgctgtagcccccggggttgggcaagggaaggatggtggccctccagaggtcatgaagggacctctgtggctccagctgccaaccctggagcccagaccgaggtggccatggagactccacctggatcccctgtaggaggccagggaggggaactcagcagttcaggagccaccccaaaccattctgggacagggacacccctttctaccccagggcagggcagggctgggtggggcaagatcccccagcccgactagacccacctcacctgaagggggtgagacccttgttggcagccagacaagggtggggctccacaggcagcacaggcgccccaccaccacccagtttggggacccagtgggaccaggtgcgggggcagagggtgacttaccaagagccagggagggcagcccaggcccaagtgacagcaagaacaagaaccactgccggcgtgcacagacttggtgtgtgtccttccctggggggacgggggactcacatgtgcctgccactggagcctctcaaccgtccagcagaacacggggttcagaaagggctccttctgctatttagcgaacactgagcatttaatttacaaatgtttgctagggtcaccctctcggccatcccacgagggtcgccatgatcaccccaactctagaggccgcagcagagctcaggacattcccccacagagcttgcccctcagttcctacctccaagggggagggtcctggaagcgcccacccaggcgccgcccctgtgcttgctccccgagctcagggattgccgagtccacgtaactgacctgtactccacgaggccctgtgggaacggtccaggctggtcctgccctgtggaggcctccgtgcactgagagatgtactaggattgcagcaaaggtggtcagggtgatgggccgcacagcgaggcagtcaaggccagctccctgggagaagcactgggtcaggtgaggtctgaggacagcaggccttccctaggggaaggagctgggagtgccaaggccccaggtgcacaggaggcgtggctgctgagaggctgcagggtggaggggcctcggcctcagagtcatgtgccctgtgaccactgaagggtgtcagcagagcacacggcatgaggacagagggaggggcacggggagtgaaggagggggccctggggcaaggctcgggggtcaggagctcagcgtccgctactcagcccagccaaaaccctcccagacgtctcctctcctgcctgggcaaagtccagcttggcaccccgtctggggcctgcctgtggtcagggccaagtgttccctcctccaggaaagcctttaccctcctcatgccctgtagtcaggaggccgcctgctgtaaccctccgtgtcgcctcgggtgcgaaatcagacccacctgacaccatcacgcggaggcccagcagcacctgcacccacttccagctgctctggccaaaatctccgctcggccaggccccgtggctcacacctgtaatcctagcacattgggaggccaaggcaggcacatcacctgagttcaggagttcaagaccagcctggccaacatggtgaaatcccgtctctactaaaaacagaaaattatccgggcgtggtggcacatgactgtaatcccagctactcaggaggctgaggcaggaggatcacttgaacctgggaggcggaggttgcagtgagctgagattgcgccattgcactccagcctgggcaacaagagcaaaattctgcctcaaaaaaaaaaatagtaataatacaaaaattagctgggcgtggtggcacatgccagtaattccatctactcgggaggctgaggcaggagaatcgtctaagcccgggaggtggaggttgcagtgagcccagatggcgctgctgcactcaagcttggatgacagagcaagactccgtttcaaaaaaaaaaaacctcctctcttccttcacaccttcctctgaatcccacccggtcccacctcctgaacctatccagacaccttctcctgacccaggcaccacctgctttcggggcgatggccgtagcctcctcccaggcacctgtctgcatccctctggccagtgcatgctgagcacgtgacctacccgtgttgggacacgtgaggatacagccttgacccccaggggctgacattctagggggagatagaaggagacaaacgtagaaggtagaataagtgggtggtggagtggcagggagtgctgagtgccacaggaagtcagacaaggaaggagagtgtggggcaggtgccgtttaaatggggggcgctggggtctcctcacagttgcttctcagctcagctgtgccaggatcttgttgagtcaggtcagctgcccacagccctcttgcctgacccctgaagcccagaactctgatcttcacagccctaggtatggccccagcaccccactgccctctctcctgccccagccgactgctgttcccagacttccctggccacgctccaagacgccagctctgccgcgggcactttgttctcacggtgtcctccatgcctgcagggcccatgcatgggaagttgcgttggcggcctgggtgttggcggttccgtgcctgctccaactctccgtgaggcccctctcccagagcctgacacactctgtggccgaactctaggcaggtgcccctgagtcctttcctcgacgaggcctgaccccatccccatcctcgctgggcccgccgaccccggtgttagcaagaatcctctaaatcagtttatggagaattacccaccctcgatatctgatcccattcctcatctcccacccttgatctcatcaccctgccggcctcctgcaagatcctcattgagccactccagtgagaatccccctaccctcgaaggccgccctaacaacttcccatccgctgacccctccaacgccatcaatctccagctgtggttgttgaactcggaggtgagctcctctcaccactctcttgaataaagcttttctcaccattttaaaaaaaaaaaaaaa

SEQ ID NO: 34 - protein, 148 aa, Homo sapiens
MARIPTAALGCISLLCLQLPGSLSRSLGGDPRPVKPREPPARSPSSSLQPRHPAPRPVVWKLHRALQAQRGAGLAPVMGQPLRDGGRQHSGPRRHSGPRRTQAQLLRVGCVLGTCQVQNLSHRLWQLMGPAGRQDSAPVDPSSPHSYG

SEQ ID NO: 35 - DNA, 3406 bp, Homo sapiens
  source 1..3406: mol_type = genomic DNA
aggcgggcggagcgaggggtgggagggcgcgcgcgaacgggcgggcgagcaagcgagcggcgtctccaccagcatctgccgcggccgcctttgcccgaagcccggggacgaaccgacggaccgaccgcctggcgcacggacgcgggcgctcgctttgtgttcggggctagcgtcggcgaggcttgagcttgcagcgcgcggcttccctgctttctcgcggccaccccggctccggcggcctcggcgcgcgaggggctggaggtgcgggagccgctctccgccggtcggtccccgcgcggctgagcccaggccgccagcgccgcggccccgtgcggtgtccctgagctcctgctccccgccgggctgctccgagcaacggtgcttcggagctccaaactcgggctgccggggcaagtgtcttcatgaacccagaggatgtccgggaagcactacaagggtcctgaagtcagttgttgcatcaaatacttcatatttggcttcaatgtcatattttggtttttgggaataacatttcttggaattggactgtgggcatggaatgaaaaaggagttctgtccaacatctcttccatcaccgatctcggcggctttgacccagtttggctcttccttgtggtgggaggagtgatgttcattttgggatttgcagggtgcattggagcgctacgggaaaacactttccttctcaagtttttttctgtgttcctgggaattattttcttcctggagctcactgccggagttctagcatttgttttcaaagactggatcaaagaccagctgtatttctttataaacaacaacatcagagcatatcgggatgacattgatttgcaaaacctcatagacttcacccaggaatattggcagtgctgtggggcttttggagctgatgattggaacctaaatatttacttcaattgcacagattccaatgcaagtcgagagcgatgtggcgttccattctcctgctgcactaaagatcccgcagaagatgtcatcaacactcagtgtggctatgatgccaggcaaaaaccagaagttgaccagcagattgtaatctacacgaaaggctgtgtgccccagtttgagaagtggttgcaggacaatttaaccatcgttgctggtattttcataggcattgcattgctgcagatatttgggatatgcctggcccagaatttggttagcgatatcgaagctgtcagggcgagctggtagaccccctgcaaccgctgctgcaagacactggacagacccagctttcgggaccctcccgcgtgccgaactgatcttcgagctgcatggacctaatcacagatgcagcctgcagtctcgcctaatggagctgccattaggggagtgtaaaactgggaaatgctgctcactgacagaattaaaaaaaaaaataaccagtatgaaagtcgttgcgccgtgaatctctactgtagccatgaatttatggacagttagatgcttaccaaaaaagaaaaaaagggagggtaggggacccagatgtacttgaatgtgcagaaaatacattcttgtcctcatcttccgtaattggagggctgggagaggcagctttgctcttcaccacaccttggacggaccaccttctttctgttccatggcctgaaggagtgcatctcctcaaagactcagcccctcacctgggagggcagtggtttgtgggcatccctccatgtacattttaggaaacacttgcaactctcatctgaagaagaaaacaactcatctttgggttcagattttgtgatggtattcagcaagtcacttgggcgagcacacttggtctatcctggaaagtctccttataagagaagttgtgtatttcatgtgcaccgagcaagggcattggaagacgtcatgaggctgtattttagcaggactgatcgtttttctaagtagacctgagctttgtttatcagtgaaattcaaggagaaaatgaggttaatgaagaggtatcagttaaatatccccttcttctcaccctgccaaaattagcagttggatttttggaaactctggaatattctgggtcattttgttttgtatgtttgttgtttttcgtcttccaaaggtgaaagctatgatacagttccacttaaattttagtgttttcttactcagctcaagcattaatttttgattaagtcttaatctgcatgacctgtgaatctgaatccatcatctccctttcctgccagcttttctacaaacattgaaatatgttatttggtcagcacttatttcctaggttcacagccttgggaggttgtggcatgtcctcccagtctggctgggaagagaccagctgtaccatccaaatgcttccctggtcttgatgatctcttccagagtcgatctgagtggccttttctgcaccctccccttctttctctttgaatggaattaaacccaatttggaaacaacattgacccagtcaaaagcttctaatggtttctttttcttcctccagttttagtttgcttttattaaaaaaagaaaatagtgcatggccatagctccttcagttctcttattgcagactaaccatcaggatggtatcaaagcacaaatactttggaggggaatgcgttgaactggggcaagtactctgtaacacaaagtgggaaaccacttcctggtgctgccgctcctgcccccactttaggtgggagggacgagttttgccctctagattttaatccagctggtgtccaccggatgttgccctcctggggagcagatatcagtctgtggaactctgggaaaaccacaggcacatttttcggtgcggacagatttgccagcacataactgggcagccagctagaatactttgtggaaattaagcgaggttttccatttcagccccatggtgcatggtggtggccgatgaatgtgtcagtctgctcagagaaaggacaaaaaggaaattattttcaaaactgtgttcactgtttgggtgtgtgtatggctctgcatgtgtgtgtttttgtctctgtataggtagaggtattcacatcttactccgactgtaaggttgtcttacttcatctctgcccccaccacagttgccattttgtaatgtccttccaacatggagaagacacgagctctctccagttggcatcatttgtcttttttgttgattgcctcattctccagtgaactccatctggccaattgattcagaatcaggcaagatccctgccctttggcacatccactgaaaggccaaacagcaagtccgagtgagttttaaatattaattaatcaccctttattttttacacttgagagtgattgtaataaaggctgtcattaataaacttggttctaccttaaaaaaaaaa

SEQ ID NO: 36 - DNA, 5882 bp, Homo sapiens
  source 1..5882: mol_type = genomic DNA
atcaaatttcaactccaggcagtccttccagccatgtgggttcagcggaaagagaagcaaaaccactcttcctaaaatgttagaagctgctcttcgcttaccttggggcctttgcattgggagctgtttttcacatcaaagaatatgtgctgaatggaattttagtattttgctgtcgttttaatattttcgtctggtcttcctcagttcttccagacgctttctgagagaatgggggcaggagctctagccatctgtcaaagtaaagcagcggttcggctgaaagaagacatgaaaaagatagtggcagtgccattaaatgaacagaaggattttacctatcagaagttatttggagtcagtctccaagaacttgaacggcaggggctcaccgagaatggcattccagcagtagtgtggaatatagtggaatatttgacgcagcatggacttacccaagaaggtctttttagggtgaatggtaacgtgaaggtggtggaacaacttcgactgaagttcgagagtggagtgcccgtggagctcgggaaggacggtgatgtctgctcagcagccagtctgttgaagctgtttctgagggagctgcctgacagtctgatcacctcagcgttgcagcctcgattcattcaactctttcaggatggcagaaatgatgttcaggagagtagcttaagagacttaataaaagagctgccagacacccactactgcctcctcaagtacctttgccagttcttgacaaaagtagccaagcatcatgtgcagaatcgcatgaatgttcacaatctcgccactgtatttgggccaaattgctttcatgtgccacctgggcttgaaggcatgaaggaacaggacctgtgcaacaagataatggctaaaattctagaaaattacaataccctgtttgaagtagagtatacagaaaatgatcatctgagatgtgaaaacctggctaggcttatcatagtaaaagaggtctattataagaactccctgcccatccttttaacaagaggcttagaaagagacatgccaaaaccacctccaaaaaccaagatcccaaaatccaggagtgagggatctattcaggcccacagagtactgcaaccagagctatctgatggcattcctcagctcagcttgcggctaagttatagaaaagcctgcttggaagacatgaattcagcagagggtgctattagtgccaagttggtacccagttcacaggaagatgaaagacctctgtcacctttctatttgagtgctcatgtaccccaagtcagcaatgtgtctgcaaccggagaactcttagaaagaaccatccgatcagctgtagaacaacatctttttgatgttaataactctggaggtcaaagttcagaggactcagaatctggaacactatcagcatcttctgccacatctgccagacagcgccgccgccagtccaaggagcaggatgaagttcgacatgggagagacaagggacttatcaacaaagaaaatactccttctgggttcaaccaccttgatgattgtattttgaatactcaggaagtcgaaaaggtacacaaaaatacttttggttgtgctggagaaaggagcaagcctaaacgtcagaaatccagtactaaactttctgagcttcatgacaatcaggacggtcttgtgaatatggaaagtctcaattccacacgatctcatgagagaactggacctgatgattttgaatggatgtctgatgaaaggaaaggaaatgaaaaagatggtggacacactcagcattttgagagccccacaatgaagatccaggagcatcccagcctatctgacaccaaacagcagagaaatcaagatgccggtgaccaggaggagagctttgtctccgaagtgccccagtcggacctgactgcattgtgtgatgaaaagaactgggaagagcctatccctgctttctcctcctggcagcgggagaacagtgactctgatgaagcccacctctcgccgcaggctgggcgcctgatccgtcagctgctggacgaagacagcgaccccatgctctctcctcggttctacgcttatgggcagagcaggcaatacctggatgacacagaagtgcctccttccccaccaaactcccattctttcatgaggcggcgaagctcctctctggggtcctatgatgatgagcaagaggacctgacacctgcccagctcacacgaaggattcagagccttaaaaagaagatccggaagtttgaagatagattcgaagaagagaagaagtacagaccttcccacagtgacaaagcagccaatccggaggttctgaaatggacaaatgaccttgccaaattccggagacaacttaaagaatcaaaactaaagatatctgaagaggacctaactcccaggatgcggcagcgaagcaacacactccccaagagttttggttcccaacttgagaaagaagatgagaagaagcaagagctggtggataaagcaataaagcccagtgttgaagccacattggaatctattcagaggaagctccaggagaagcgagcggaaagcagccgccctgaggacattaaggatatgaccaaagaccagattgctaatgagaaagtggctctgcagaaagctctgttatattatgaaagcattcatggacggccggtaacaaagaacgaacggcaggtgatgaagccactatacgacaggtaccggctggtcaaacagatcctctcccgagctaacaccatacccatcattggttccccctccagcaagcggagaagccctttgctgcagccaattatcgagggcgaaactgcttccttcttcaaggagataaaggaagaagaggaggggtcagaagacgatagcaatgtgaagccagacttcatggtcactctgaaaaccgatttcagtgcacgatgctttctggaccaattcgaagatgacgctgatggatttatttccccaatggatgataaaataccatcaaaatgcagccaggacacagggctttcaaatctccatgctgcctcaatacctgaactcctggaacacctccaggaaatgagagaagaaaagaaaaggattcgaaagaaacttcgggattttgaagacaactttttcagacagaatggaagaaatgtccagaaggaagaccgcactcctatggctgaagaatacagtgaatataagcacataaaggcgaaactgaggctcctggaggtgctcatcagcaagagagacactgattccaagtccatgtgaggggcatggccaagcacagggggctggcagctgcggtgagagtttactgtccccagagaaagtgcagctctggaaggcagccttggggctggccctgcaaagcatgcagcccttctgcctctagaccatttggcatcggctcctgtttccattgcctgccttagaaactggctggaagaagacaatgtgacctgacttaggcattttgtaattggaaagtcaagactgcagtatgtgcacatgcgcacgcgcatgcacgcacacacacacacagtagtggagctttcctaacactagcagagattaatcactacattagacaacactcatctacagagaatatacactgttcttccctggataactgagaaacaagagaccattctctgtctaactgtgataaaaacaagctcaggactttattctatagagcaaacttgctgtggagggccatgctctccttggacccagttaactgcaaacgtgcattggagccctatttgctgccgctgccattctagtgacctttccacagagctgcgccttcctcacgtgtgtgaaaggttttccccttcagccctcaggtagatggaagctgcatctgcccacgatggcagtgcagtcatcatcttcaggatgtttcttcaggacttcctcagctgacaaggaattttggtccctgcctaggaccgggtcatctgcagaggacagagagatggtaagcagctgtatgaatgctgattttaaaaccaggtcatgggagaagagcctggagattctttcctgaacactgactgcacttaccagtctgattttatcgtcaaacaccaagccaggctagcatgctcatggcaatctgtttggggctgttttgttgtggcactagccaaacataaaggggcttaagtcagcctgcatacagaggatcggggagagaaggggcctgtgttctcagcctcctgagtacttaccagagtttaatttttttaaaaaaaatctgcactaaaatccccaaactgacaggtaaatgtagccctcagagctcagcccaaggcagaatctaaatcacactattttcgagatcatgtataaaaagaaaaaaaagaagtcatgctgtgtggccaattataatttttttcaaagactttgtcacaaaactgtctatattagacattttggagggaccaggaaatgtaagacaccaaatcctccatctcttcagtgtgcctgatgtcacctcatgatttgctgttacttttttaactcctgcgccaaggacagtgggttctgtgtccacctttgtgctttgcgaggccgagcccaggcatctgctcgcctgccacggctgaccagagaaggtgcttcaggagctctgccttagacgacgtgttacagtatgaacacacagcagaggcaccctcgtatgttttgaaagttgccttctgaaagggcacagttttaaggaaaagaaaaagaatgtaaaactatactgacccgttttcagttttaaagggtcgtgagaaactggctggtccaatgggatttacagcaacattttccattgctgaagtgaggtagcagctctcttctgtcagctgaatgttaaggatggggaaaaagaatgcctttaagtttgctcttaatcgtatggaagcttgagctatgtgttggaagtgccctggttttaatccatacacaaagacggtacataatcctacaggtttaaatgtacataaaaatatagtttggaattctttgctctactgtttacattgcagattgctataatttcaaggagtgagattataaataaaatgatgcactttaggatgtttcctatttttgaaatctgaacatgaatcattcacatgaccaaaaattgtgtttttttaaaaatacatgtctagtctgtcctttaatagctctcttaaataagctatgatattaatcagatcattaccagttagcttttaaagcacatttgtttaagactatgtttttggaaaaatacgctacagaatttttttttaagctacaaataaatgagatgctactaattgttttggaatctgttgtttctgccaaaggtaaattaactaaagatttattcaggaatccccatttgaatttgtatgattcaataaaagaaaacaccaagtaagttatataaaataaattgtgtatgagatgttgtgttttcctttgtaatttccactaactaactaactaacttatattcttcatggaatggagcccagaagaaatgagaggaagcccttttcacactagatcttatttgaagaaatgtttgttagtcagtcagtcagtggtttctggctctgccgagggagatgtgttccccagcaaccatttctgcagcccagaatctcaaggcactagaggcggtgtcttaattaattggcttcacaaagacaaaatgctctggactgggatttttcctttgctgtgttgggaatatgtgtttattaattagcacatgccaacaaaataaatgtcaagagttatttcataagtgtaagtaaacttaagaattaaagagtgcagacttataattttca

SEQ ID NO: 37 - protein, 1023 aa, Homo sapiens
MGAGALAICQSKAAVRLKEDMKKIVAVPLNEQKDFTYQKLFGVSLQELERQGLTENGIPAVVWNIVEYLTQHGLTQEGLFRVNGNVKVVEQLRLKFESGVPVELGKDGDVCSAASLLKLFLRELPDSLITSALQPRFIQLFQDGRNDVQESSLRDLIKELPDTHYCLLKYLCQFLTKVAKHHVQNRMNVHNLATVFGPNCFHVPPGLEGMKEQDLCNKIMAKILENYNTLFEVEYTENDHLRCENLARLIIVKEVYYKNSLPILLTRGLERDMPKPPPKTKIPKSRSEGSIQAHRVLQPELSDGIPQLSLRLSYRKACLEDMNSAEGAISAKLVPSSQEDERPLSPFYLSAHVPQVSNVSATGELLERTIRSAVEQHLFDVNNSGGQSSEDSESGTLSASSATSARQRRRQSKEQDEVRHGRDKGLINKENTPSGFNHLDDCILNTQEVEKVHKNTFGCAGERSKPKRQKSSTKLSELHDNQDGLVNMESLNSTRSHERTGPDDFEWMSDERKGNEKDGGHTQHFESPTMKIQEHPSLSDTKQQRNQDAGDQEESFVSEVPQSDLTALCDEKNWEEPIPAFSSWQRENSDSDEAHLSPQAGRLIRQLLDEDSDPMLSPRFYAYGQSRQYLDDTEVPPSPPNSHSFMRRRSSSLGSYDDEQEDLTPAQLTRRIQSLKKKIRKFEDRFEEEKKYRPSHSDKAANPEVLKWTNDLAKFRRQLKESKLKISEEDLTPRMRQRSNTLPKSFGSQLEKEDEKKQELVDKAIKPSVEATLESIQRKLQEKRAESSRPEDIKDMTKDQIANEKVALQKALLYYESIHGRPVTKNERQVMKPLYDRYRLVKQILSRANTIPIIGSPSSKRRSPLLQPIIEGETASFFKEIKEEEEGSEDDSNVKPDFMVTLKTDFSARCFLDQFEDDADGFISPMDDKIPSKCSQDTGLSNLHAASIPELLEHLQEMREEKKRIRKKLRDFEDNFFRQNGRNVQKEDRTPMAEEYSEYKHIKAKLRLLEVLISKRDTDSKSM

SEQ ID NO: 38 - DNA, 4988 bp, Homo sapiens
  source 1..4988: mol_type = genomic DNA
attgaggagcagaaggagtagggtgcgggggaggaggaggagcgcctttagtgctgcagcagctgctgctctgattggcccggtggttcagctgcttccctggaacaaaaggtcaaagtggactgcagtgtaaatgtagagaagcagccgataaaatagcattgcctgaagaagtttggaggctgagagcagcagtagactggccaactgcagagcaagttgtttctccagccgtgcggtgcagcctcatgcccccaacccagcttagccactgtaagaagacgttcactgtacagacgaccaaacttgccgtggaagagacagttgtgagattcccttgcaaatttacatacgagaatggcttgtgaaatcatgcctctgcaaagtgctcatgtaccccaagtcagcaatgtgtctgcaaccggagaactcttagaaagaaccatccgatcagctgtagaacaacatctttttgatgttaataactctggaggtcaaagttcagaggactcagaatctggaacactatcagcatcttctgccacatctgccagacagcgccgccgccagtccaaggagcaggatgaagttcgacatgggagagacaagggacttatcaacaaagaaaatactccttctgggttcaaccaccttgatgattgtattttgaatactcaggaagtcgaaaaggtacacaaaaatacttttggttgtgctggagaaaggagcaagcctaaacgtcagaaatccagtactaaactttctgagcttcatgacaatcaggacggtcttgtgaatatggaaagtctcaattccacacgatctcatgagagaactggacctgatgattttgaatggatgtctgatgaaaggaaaggaaatgaaaaagatggtggacacactcagcattttgagagccccacaatgaagatccaggagcatcccagcctatctgacaccaaacagcagagaaatcaagatgccggtgaccaggaggagagctttgtctccgaagtgccccagtcggacctgactgcattgtgtgatgaaaagaactgggaagagcctatccctgctttctcctcctggcagcgggagaacagtgactctgatgaagcccacctctcgccgcaggctgggcgcctgatccgtcagctgctggacgaagacagcgaccccatgctctctcctcggttctacgcttatgggcagagcaggcaatacctggatgacacagaagtgcctccttccccaccaaactcccattctttcatgaggcggcgaagctcctctctggggtcctatgatgatgagcaagaggacctgacacctgcccagctcacacgaaggattcagagccttaaaaagaagatccggaagtttgaagatagattcgaagaagagaagaagtacagaccttcccacagtgacaaagcagccaatccggaggttctgaaatggacaaatgaccttgccaaattccggagacaacttaaagaatcaaaactaaagatatctgaagaggacctaactcccaggatgcggcagcgaagcaacacactccccaagagttttggttcccaacttgagaaagaagatgagaagaagcaagagctggtggataaagcaataaagcccagtgttgaagccacattggaatctattcagaggaagctccaggagaagcgagcggaaagcagccgccctgaggacattaaggatatgaccaaagaccagattgctaatgagaaagtggctctgcagaaagctctgttatattatgaaagcattcatggacggccggtaacaaagaacgaacggcaggtgatgaagccactatacgacaggtaccggctggtcaaacagatcctctcccgagctaacaccatacccatcattggttccccctccagcaagcggagaagccctttgctgcagccaattatcgagggcgaaactgcttccttcttcaaggagataaaggaagaagaggaggggtcagaagacgatagcaatgtgaagccagacttcatggtcactctgaaaaccgatttcagtgcacgatgctttctggaccaattcgaagatgacgctgatggatttatttccccaatggatgataaaataccatcaaaatgcagccaggacacagggctttcaaatctccatgctgcctcaatacctgaactcctggaacacctccaggaaatgagagaagaaaagaaaaggattcgaaagaaacttcgggattttgaagacaactttttcagacagaatggaagaaatgtccagaaggaagaccgcactcctatggctgaagaatacagtgaatataagcacataaaggcgaaactgaggctcctggaggtgctcatcagcaagagagacactgattccaagtccatgtgaggggcatggccaagcacagggggctggcagctgcggtgagagtttactgtccccagagaaagtgcagctctggaaggcagccttggggctggccctgcaaagcatgcagcccttctgcctctagaccatttggcatcggctcctgtttccattgcctgccttagaaactggctggaagaagacaatgtgacctgacttaggcattttgtaattggaaagtcaagactgcagtatgtgcacatgcgcacgcgcatgcacgcacacacacacacagtagtggagctttcctaacactagcagagattaatcactacattagacaacactcatctacagagaatatacactgttcttccctggataactgagaaacaagagaccattctctgtctaactgtgataaaaacaagctcaggactttattctatagagcaaacttgctgtggagggccatgctctccttggacccagttaactgcaaacgtgcattggagccctatttgctgccgctgccattctagtgacctttccacagagctgcgccttcctcacgtgtgtgaaaggttttccccttcagccctcaggtagatggaagctgcatctgcccacgatggcagtgcagtcatcatcttcaggatgtttcttcaggacttcctcagctgacaaggaattttggtccctgcctaggaccgggtcatctgcagaggacagagagatggtaagcagctgtatgaatgctgattttaaaaccaggtcatgggagaagagcctggagattctttcctgaacactgactgcacttaccagtctgattttatcgtcaaacaccaagccaggctagcatgctcatggcaatctgtttggggctgttttgttgtggcactagccaaacataaaggggcttaagtcagcctgcatacagaggatcggggagagaaggggcctgtgttctcagcctcctgagtacttaccagagtttaatttttttaaaaaaaatctgcactaaaatccccaaactgacaggtaaatgtagccctcagagctcagcccaaggcagaatctaaatcacactattttcgagatcatgtataaaaagaaaaaaaagaagtcatgctgtgtggccaattataatttttttcaaagactttgtcacaaaactgtctatattagacattttggagggaccaggaaatgtaagacaccaaatcctccatctcttcagtgtgcctgatgtcacctcatgatttgctgttacttttttaactcctgcgccaaggacagtgggttctgtgtccacctttgtgctttgcgaggccgagcccaggcatctgctcgcctgccacggctgaccagagaaggtgcttcaggagctctgccttagacgacgtgttacagtatgaacacacagcagaggcaccctcgtatgttttgaaagttgccttctgaaagggcacagttttaaggaaaagaaaaagaatgtaaaactatactgacccgttttcagttttaaagggtcgtgagaaactggctggtccaatgggatttacagcaacattttccattgctgaagtgaggtagcagctctcttctgtcagctgaatgttaaggatggggaaaaagaatgcctttaagtttgctcttaatcgtatggaagcttgagctatgtgttggaagtgccctggttttaatccatacacaaagacggtacataatcctacaggtttaaatgtacataaaaatatagtttggaattctttgctctactgtttacattgcagattgctataatttcaaggagtgagattataaataaaatgatgcactttaggatgtttcctatttttgaaatctgaacatgaatcattcacatgaccaaaaattgtgtttttttaaaaatacatgtctagtctgtcctttaatagctctcttaaataagctatgatattaatcagatcattaccagttagcttttaaagcacatttgtttaagactatgtttttggaaaaatacgctacagaatttttttttaagctacaaataaatgagatgctactaattgttttggaatctgttgtttctgccaaaggtaaattaactaaagatttattcaggaatccccatttgaatttgtatgattcaataaaagaaaacaccaagtaagttatataaaataaattgtgtatgagatgttgtgttttcctttgtaatttccactaactaactaactaacttatattcttcatggaatggagcccagaagaaatgagaggaagcccttttcacactagatcttatttgaagaaatgtttgttagtcagtcagtcagtggtttctggctctgccgagggagatgtgttccccagcaaccatttctgcagcccagaatctcaaggcactagaggcggtgtcttaattaattggcttcacaaagacaaaatgctctggactgggatttttcctttgctgtgttgggaatatgtgtttattaattagcacatgccaacaaaataaatgtcaagagttatttcataagtgtaagtaaacttaagaattaaagagtgcagacttataattttca

SEQ ID NO: 39 - protein, 683 aa, Homo sapiens
MACEIMPLQSAHVPQVSNVSATGELLERTIRSAVEQHLFDVNNSGGQSSEDSESGTLSASSATSARQRRRQSKEQDEVRHGRDKGLINKENTPSGFNHLDDCILNTQEVEKVHKNTFGCAGERSKPKRQKSSTKLSELHDNQDGLVNMESLNSTRSHERTGPDDFEWMSDERKGNEKDGGHTQHFESPTMKIQEHPSLSDTKQQRNQDAGDQEESFVSEVPQSDLTALCDEKNWEEPIPAFSSWQRENSDSDEAHLSPQAGRLIRQLLDEDSDPMLSPRFYAYGQSRQYLDDTEVPPSPPNSHSFMRRRSSSLGSYDDEQEDLTPAQLTRRIQSLKKKIRKFEDRFEEEKKYRPSHSDKAANPEVLKWTNDLAKFRRQLKESKLKISEEDLTPRMRQRSNTLPKSFGSQLEKEDEKKQELVDKAIKPSVEATLESIQRKLQEKRAESSRPEDIKDMTKDQIANEKVALQKALLYYESIHGRPVTKNERQVMKPLYDRYRLVKQILSRANTIPIIGSPSSKRRSPLLQPIIEGETASFFKEIKEEEEGSEDDSNVKPDFMVTLKTDFSARCFLDQFEDDADGFISPMDDKIPSKCSQDTGLSNLHAASIPELLEHLQEMREEKKRIRKKLRDFEDNFFRQNGRNVQKEDRTPMAEEYSEYKHIKAKLRLLEVLISKRDTDSKSM

SEQ ID NO: 40 - DNA, 4946 bp, Homo sapiens
  source 1..4946: mol_type = genomic DNA
attgaggagcagaaggagtagggtgcgggggaggaggaggagcgcctttagtgctgcagcagctgctgctctgattggcccggtggttcagctgcttccctggaacaaaaggtcaaagtggactgcagtgtaaatgtagagaagcagccgataaaatagcattgcctgaagaagtttggaggctgagagcagcagtagactggccaactgcagagcaagttgtttctccagccgtgcggtgcagcctcatgcccccaacccagcttagccactgtaagaagacgttcactgtacagacgaccaaacttgccgtggaagagacagttgtgagattcccttgcaaatttacatacgagaatggcttgtgaaatcatgcctctgcaaagttcacaggaagatgaaagacctctgtcacctttctatttgagtgctcatgtaccccaagtcagcaatgtgtctgcaaccggagaactcttagaaagaaccatccgatcagctgtagaacaacatctttttgatgttaataactctggaggtcaaagttcagaggactcagaatctggaacactatcagcatcttctgccacatctgccagacagcgccgccgccagtccaaggagcaggatgaagttcgacatgggagagacaagggacttatcaacaaagaaaatactccttctgggttcaaccaccttgatgattgtattttgaatactcaggaagtcgaaaaggtacacaaaaatacttttggttgtgctggagaaaggagcaagcctaaacgtcagaaatccagtactaaactttctgagcttcatgacaatcaggacggtcttgtgaatatggaaagtctcaattccacacgatctcatgagagaactggacctgatgattttgaatggatgtctgatgaaaggaaaggaaatgaaaaagatggtggacacactcagcattttgagagccccacaatgaagatccaggagcatcccagcctatctgacaccaaacagcagagaaatcaagatgccggtgaccaggaggagagctttgtctccgaagtgccccagtcggacctgactgcattgtgtgatgaaaagaactgggaagagcctatccctgctttctcctcctggcagcgggagaacagtgactctgatgaagcccacctctcgccgcaggctgggcgcctgatccgtcagctgctggacgaagacagcgaccccatgctctctcctcggttctacgcttatgggcagagcaggcaatacctggatgacacagaagtgcctccttccccaccaaactcccattctttcatgaggcggcgaagctcctctctggggtcctatgatgatgagcaagaggacctgacacctgcccagctcacacgaaggattcagagccttaaaaagaagatccggaagtttgaagatagattcgaagaagagaagaagtacagaccttcccacagtgacaaagcagccaatccggaggttctgaaatggacaaatgaccttgccaaattccggagacaacttaaagaatcaaaactaaagatatctgaagaggacctaactcccaggatgcggcagcgaagcaacacactccccaagagttttggttcccaacttgagaaagaagatgagaagaagcaagagctggtggataaagcaataaagcccagtgttgaagccacattggaatctattcagaggaagctccaggagaagcgagcggaaagcagccgccctgaggacattaaggatatgaccaaagaccagattgctaatgagaaagtggctctgcagaaagctctgttatattatgaaagcattcatggacggccggtaacaaagaacgaacggcaggtgatgaagccactatacgacaggtaccggctggtcaaacagatcctctcccgagctaacaccatacccatcattgaagaagaggaggggtcagaagacgatagcaatgtgaagccagacttcatggtcactctgaaaaccgatttcagtgcacgatgctttctggaccaattcgaagatgacgctgatggatttatttccccaatggatgataaaataccatcaaaatgcagccaggacacagggctttcaaatctccatgctgcctcaatacctgaactcctggaacacctccaggaaatgagagaagaaaagaaaaggattcgaaagaaacttcgggattttgaagacaactttttcagacagaatggaagaaatgtccagaaggaagaccgcactcctatggctgaagaatacagtgaatataagcacataaaggcgaaactgaggctcctggaggtgctcatcagcaagagagacactgattccaagtccatgtgaggggcatggccaagcacagggggctggcagctgcggtgagagtttactgtccccagagaaagtgcagctctggaaggcagccttggggctggccctgcaaagcatgcagcccttctgcctctagaccatttggcatcggctcctgtttccattgcctgccttagaaactggctggaagaagacaatgtgacctgacttaggcattttgtaattggaaagtcaagactgcagtatgtgcacatgcgcacgcgcatgcacgcacacacacacacagtagtggagctttcctaacactagcagagattaatcactacattagacaacactcatctacagagaatatacactgttcttccctggataactgagaaacaagagaccattctctgtctaactgtgataaaaacaagctcaggactttattctatagagcaaacttgctgtggagggccatgctctccttggacccagttaactgcaaacgtgcattggagccctatttgctgccgctgccattctagtgacctttccacagagctgcgccttcctcacgtgtgtgaaaggttttccccttcagccctcaggtagatggaagctgcatctgcccacgatggcagtgcagtcatcatcttcaggatgtttcttcaggacttcctcagctgacaaggaattttggtccctgcctaggaccgggtcatctgcagaggacagagagatggtaagcagctgtatgaatgctgattttaaaaccaggtcatgggagaagagcctggagattctttcctgaacactgactgcacttaccagtctgattttatcgtcaaacaccaagccaggctagcatgctcatggcaatctgtttggggctgttttgttgtggcactagccaaacataaaggggcttaagtcagcctgcatacagaggatcggggagagaaggggcctgtgttctcagcctcctgagtacttaccagagtttaatttttttaaaaaaaatctgcactaaaatccccaaactgacaggtaaatgtagccctcagagctcagcccaaggcagaatctaaatcacactattttcgagatcatgtataaaaagaaaaaaaagaagtcatgctgtgtggccaattataatttttttcaaagactttgtcacaaaactgtctatattagacattttggagggaccaggaaatgtaagacaccaaatcctccatctcttcagtgtgcctgatgtcacctcatgatttgctgttacttttttaactcctgcgccaaggacagtgggttctgtgtccacctttgtgctttgcgaggccgagcccaggcatctgctcgcctgccacggctgaccagagaaggtgcttcaggagctctgccttagacgacgtgttacagtatgaacacacagcagaggcaccctcgtatgttttgaaagttgccttctgaaagggcacagttttaaggaaaagaaaaagaatgtaaaactatactgacccgttttcagttttaaagggtcgtgagaaactggctggtccaatgggatttacagcaacattttccattgctgaagtgaggtagcagctctcttctgtcagctgaatgttaaggatggggaaaaagaatgcctttaagtttgctcttaatcgtatggaagcttgagctatgtgttggaagtgccctggttttaatccatacacaaagacggtacataatcctacaggtttaaatgtacataaaaatatagtttggaattctttgctctactgtttacattgcagattgctataatttcaaggagtgagattataaataaaatgatgcactttaggatgtttcctatttttgaaatctgaacatgaatcattcacatgaccaaaaattgtgtttttttaaaaatacatgtctagtctgtcctttaatagctctcttaaataagctatgatattaatcagatcattaccagttagcttttaaagcacatttgtttaagactatgtttttggaaaaatacgctacagaatttttttttaagctacaaataaatgagatgctactaattgttttggaatctgttgtttctgccaaaggtaaattaactaaagatttattcaggaatccccatttgaatttgtatgattcaataaaagaaaacaccaagtaagttatataaaataaattgtgtatgagatgttgtgttttcctttgtaatttccactaactaactaactaacttatattcttcatggaatggagcccagaagaaatgagaggaagcccttttcacactagatcttatttgaagaaatgtttgttagtcagtcagtcagtggtttctggctctgccgagggagatgtgttccccagcaaccatttctgcagcccagaatctcaaggcactagaggcggtgtcttaattaattggcttcacaaagacaaaatgctctggactgggatttttcctttgctgtgttgggaatatgtgtttattaattagcacatgccaacaaaataaatgtcaagagttatttcataagtgtaagtaaacttaagaattaaagagtgcagacttataattttca

SEQ ID NO: 41 - protein, 669 aa, Homo sapiens
MACEIMPLQSSQEDERPLSPFYLSAHVPQVSNVSATGELLERTIRSAVEQHLFDVNNSGGQSSEDSESGTLSASSATSARQRRRQSKEQDEVRHGRDKGLINKENTPSGFNHLDDCILNTQEVEKVHKNTFGCAGERSKPKRQKSSTKLSELHDNQDGLVNMESLNSTRSHERTGPDDFEWMSDERKGNEKDGGHTQHFESPTMKIQEHPSLSDTKQQRNQDAGDQEESFVSEVPQSDLTALCDEKNWEEPIPAFSSWQRENSDSDEAHLSPQAGRLIRQLLDEDSDPMLSPRFYAYGQSRQYLDDTEVPPSPPNSHSFMRRRSSSLGSYDDEQEDLTPAQLTRRIQSLKKKIRKFEDRFEEEKKYRPSHSDKAANPEVLKWTNDLAKFRRQLKESKLKISEEDLTPRMRQRSNTLPKSFGSQLEKEDEKKQELVDKAIKPSVEATLESIQRKLQEKRAESSRPEDIKDMTKDQIANEKVALQKALLYYESIHGRPVTKNERQVMKPLYDRYRLVKQILSRANTIPIIEEEEGSEDDSNVKPDFMVTLKTDFSARCFLDQFEDDADGFISPMDDKIPSKCSQDTGLSNLHAASIPELLEHLQEMREEKKRIRKKLRDFEDNFFRQNGRNVQKEDRTPMAEEYSEYKHIKAKLRLLEVLISKRDTDSKSM

SEQ ID NO: 42 - DNA, 4946 bp, Homo sapiens
  source 1..4946: mol_type = genomic DNA
attgaggagcagaaggagtagggtgcgggggaggaggaggagcgcctttagtgctgcagcagctgctgctctgattggcccggtggttcagctgcttccctggaacaaaaggtcaaagtggactgcagtgtaaatgtagagaagcagccgataaaatagcattgcctgaagaagtttggaggctgagagcagcagtagactggccaactgcagagcaagttgtttctccagccgtgcggtgcagcctcatgcccccaacccagcttagccactgtaagaagacgttcactgtacagacgaccaaacttgccgtggaagagacagttgtgagattcccttgcaaatttacatacgagaatggcttgtgaaatcatgcctctgcaaagactcttagaaagaaccatccgatcagctgtagaacaacatctttttgatgttaataactctggaggtcaaagttcagaggactcagaatctggaacactatcagcatcttctgccacatctgccagacagcgccgccgccagtccaaggagcaggatgaagttcgacatgggagagacaagggacttatcaacaaagaaaatactccttctgggttcaaccaccttgatgattgtattttgaatactcaggaagtcgaaaaggtacacaaaaatacttttggttgtgctggagaaaggagcaagcctaaacgtcagaaatccagtactaaactttctgagcttcatgacaatcaggacggtcttgtgaatatggaaagtctcaattccacacgatctcatgagagaactggacctgatgattttgaatggatgtctgatgaaaggaaaggaaatgaaaaagatggtggacacactcagcattttgagagccccacaatgaagatccaggagcatcccagcctatctgacaccaaacagcagagaaatcaagatgccggtgaccaggaggagagctttgtctccgaagtgccccagtcggacctgactgcattgtgtgatgaaaagaactgggaagagcctatccctgctttctcctcctggcagcgggagaacagtgactctgatgaagcccacctctcgccgcaggctgggcgcctgatccgtcagctgctggacgaagacagcgaccccatgctctctcctcggttctacgcttatgggcagagcaggcaatacctggatgacacagaagtgcctccttccccaccaaactcccattctttcatgaggcggcgaagctcctctctggggtcctatgatgatgagcaagaggacctgacacctgcccagctcacacgaaggattcagagccttaaaaagaagatccggaagtttgaagatagattcgaagaagagaagaagtacagaccttcccacagtgacaaagcagccaatccggaggttctgaaatggacaaatgaccttgccaaattccggagacaacttaaagaatcaaaactaaagatatctgaagaggacctaactcccaggatgcggcagcgaagcaacacactccccaagagttttggttcccaacttgagaaagaagatgagaagaagcaagagctggtggataaagcaataaagcccagtgttgaagccacattggaatctattcagaggaagctccaggagaagcgagcggaaagcagccgccctgaggacattaaggatatgaccaaagaccagattgctaatgagaaagtggctctgcagaaagctctgttatattatgaaagcattcatggacggccggtaacaaagaacgaacggcaggtgatgaagccactatacgacaggtaccggctggtcaaacagatcctctcccgagctaacaccatacccatcattggttccccctccagcaagcggagaagccctttgctgcagccaattatcgagggcgaaactgcttccttcttcaaggagataaaggaagaagaggaggggtcagaagacgatagcaatgtgaagccagacttcatggtcactctgaaaaccgatttcagtgcacgatgctttctggaccaattcgaagatgacgctgatggatttatttccccaatggatgataaaataccatcaaaatgcagccaggacacagggctttcaaatctccatgctgcctcaatacctgaactcctggaacacctccaggaaatgagagaagaaaagaaaaggattcgaaagaaacttcgggattttgaagacaactttttcagacagaatggaagaaatgtccagaaggaagaccgcactcctatggctgaagaatacagtgaatataagcacataaaggcgaaactgaggctcctggaggtgctcatcagcaagagagacactgattccaagtccatgtgaggggcatggccaagcacagggggctggcagctgcggtgagagtttactgtccccagagaaagtgcagctctggaaggcagccttggggctggccctgcaaagcatgcagcccttctgcctctagaccatttggcatcggctcctgtttccattgcctgccttagaaactggctggaagaagacaatgtgacctgacttaggcattttgtaattggaaagtcaagactgcagtatgtgcacatgcgcacgcgcatgcacgcacacacacacacagtagtggagctttcctaacactagcagagattaatcactacattagacaacactcatctacagagaatatacactgttcttccctggataactgagaaacaagagaccattctctgtctaactgtgataaaaacaagctcaggactttattctatagagcaaacttgctgtggagggccatgctctccttggacccagttaactgcaaacgtgcattggagccctatttgctgccgctgccattctagtgacctttccacagagctgcgccttcctcacgtgtgtgaaaggttttccccttcagccctcaggtagatggaagctgcatctgcccacgatggcagtgcagtcatcatcttcaggatgtttcttcaggacttcctcagctgacaaggaattttggtccctgcctaggaccgggtcatctgcagaggacagagagatggtaagcagctgtatgaatgctgattttaaaaccaggtcatgggagaagagcctggagattctttcctgaacactgactgcacttaccagtctgattttatcgtcaaacaccaagccaggctagcatgctcatggcaatctgtttggggctgttttgttgtggcactagccaaacataaaggggcttaagtcagcctgcatacagaggatcggggagagaaggggcctgtgttctcagcctcctgagtacttaccagagtttaatttttttaaaaaaaatctgcactaaaatccccaaactgacaggtaaatgtagccctcagagctcagcccaaggcagaatctaaatcacactattttcgagatcatgtataaaaagaaaaaaaagaagtcatgctgtgtggccaattataatttttttcaaagactttgtcacaaaactgtctatattagacattttggagggaccaggaaatgtaagacaccaaatcctccatctcttcagtgtgcctgatgtcacctcatgatttgctgttacttttttaactcctgcgccaaggacagtgggttctgtgtccacctttgtgctttgcgaggccgagcccaggcatctgctcgcctgccacggctgaccagagaaggtgcttcaggagctctgccttagacgacgtgttacagtatgaacacacagcagaggcaccctcgtatgttttgaaagttgccttctgaaagggcacagttttaaggaaaagaaaaagaatgtaaaactatactgacccgttttcagttttaaagggtcgtgagaaactggctggtccaatgggatttacagcaacattttccattgctgaagtgaggtagcagctctcttctgtcagctgaatgttaaggatggggaaaaagaatgcctttaagtttgctcttaatcgtatggaagcttgagctatgtgttggaagtgccctggttttaatccatacacaaagacggtacataatcctacaggtttaaatgtacataaaaatatagtttggaattctttgctctactgtttacattgcagattgctataatttcaaggagtgagattataaataaaatgatgcactttaggatgtttcctatttttgaaatctgaacatgaatcattcacatgaccaaaaattgtgtttttttaaaaatacatgtctagtctgtcctttaatagctctcttaaataagctatgatattaatcagatcattaccagttagcttttaaagcacatttgtttaagactatgtttttggaaaaatacgctacagaatttttttttaagctacaaataaatgagatgctactaattgttttggaatctgttgtttctgccaaaggtaaattaactaaagatttattcaggaatccccatttgaatttgtatgattcaataaaagaaaacaccaagtaagttatataaaataaattgtgtatgagatgttgtgttttcctttgtaatttccactaactaactaactaacttatattcttcatggaatggagcccagaagaaatgagaggaagcccttttcacactagatcttatttgaagaaatgtttgttagtcagtcagtcagtggtttctggctctgccgagggagatgtgttccccagcaaccatttctgcagcccagaatctcaaggcactagaggcggtgtcttaattaattggcttcacaaagacaaaatgctctggactgggatttttcctttgctgtgttgggaatatgtgtttattaattagcacatgccaacaaaataaatgtcaagagttatttcataagtgtaagtaaacttaagaattaaagagtgcagacttataattttca

SEQ ID NO: 43 - protein, 669 aa, Homo sapiens
MACEIMPLQRLLERTIRSAVEQHLFDVNNSGGQSSEDSESGTLSASSATSARQRRRQSKEQDEVRHGRDKGLINKENTPSGFNHLDDCILNTQEVEKVHKNTFGCAGERSKPKRQKSSTKLSELHDNQDGLVNMESLNSTRSHERTGPDDFEWMSDERKGNEKDGGHTQHFESPTMKIQEHPSLSDTKQQRNQDAGDQEESFVSEVPQSDLTALCDEKNWEEPIPAFSSWQRENSDSDEAHLSPQAGRLIRQLLDEDSDPMLSPRFYAYGQSRQYLDDTEVPPSPPNSHSFMRRRSSSLGSYDDEQEDLTPAQLTRRIQSLKKKIRKFEDRFEEEKKYRPSHSDKAANPEVLKWTNDLAKFRRQLKESKLKISEEDLTPRMRQRSNTLPKSFGSQLEKEDEKKQELVDKAIKPSVEATLESIQRKLQEKRAESSRPEDIKDMTKDQIANEKVALQKALLYYESIHGRPVTKNERQVMKPLYDRYRLVKQILSRANTIPIIGSPSSKRRSPLLQPIIEGETASFFKEIKEEEEGSEDDSNVKPDFMVTLKTDFSARCFLDQFEDDADGFISPMDDKIPSKCSQDTGLSNLHAASIPELLEHLQEMREEKKRIRKKLRDFEDNFFRQNGRNVQKEDRTPMAEEYSEYKHIKAKLRLLEVLISKRDTDSKSM

SEQ ID NO: 44 - DNA, 9802 bp, Homo sapiens
  source 1..9802: mol_type = genomic DNA
aagaaaccggccaggtgtggcctaggcgcccagtgccagcggggaggagactcgctccgccgccgaccaacaccaacacccagctccgacgcagctcctctgcgcccttgccgccctccgagccacagctttcctcccgctcctgcccccggcccgtcgccgtctccgcgctcgcagcggcctcgggagggcccaggtagcgagcagcgacctcgcgagccttccgcactcccgcccggttccccggccgtccgcctatccttggccccctccgctttctccgcgccggcccgcctcgcttatgcctcggcgctgagccgctctcccgattgcccgccgacatgagctgcaacggaggctcccacccgcggatcaacactctgggccgcatgatccgcgccgagtctggcccggacctgcgctacgaggtgaccagcggcggcgggggcaccagcaggatgtactattctcggcgcggcgtgatcaccgaccagaactcggacggctactgtcaaaccggcacgatgtccaggcaccagaaccagaacaccatccaggagctgctgcagaactgctccgactgcttgatgcgagcagagctcatcgtgcagcctgaattgaagtatggagatggaatacaactgactcggagtcgagaattggatgagtgttttgcccaggccaatgaccaaatggaaatcctcgacagcttgatcagagagatgcggcagatgggccagccctgtgatgcttaccagaaaaggcttcttcagctccaagagcaaatgcgagccctttataaagccatcagtgtccctcgagtccgcagggccagctccaagggtggtggaggctacacttgtcagagtggctctggctgggatgagttcaccaaacatgtcaccagtgaatgtttggggtggatgaggcagcaaagggcggagatggacatggtggcctggggtgtggacctggcctcagtggagcagcacattaacagccaccggggcatccacaactccatcggcgactatcgctggcagctggacaaaatcaaagccgacctgcgcgagaaatctgcgatctaccagttggaggaggagtatgaaaacctgctgaaagcgtcctttgagaggatggatcacctgcgacagctgcagaacatcattcaggccacgtccagggagatcatgtggatcaatgactgcgaggaggaggagctgctgtacgactggagcgacaagaacaccaacatcgctcagaaacaggaggccttctccatacgcatgagtcaactggaagttaaagaaaaagagctcaataagctgaaacaagaaagtgaccaacttgtcctcaatcagcatccagcttcagacaaaattgaggcctatatggacactctgcagacgcagtggagttggattcttcagatcaccaagtgcattgatgttcatctgaaagaaaatgctgcctactttcagttttttgaagaggcgcagtctactgaagcatacctgaaggggctccaggactccatcaggaagaagtacccctgcgacaagaacatgcccctgcagcacctgctggaacagatcaaggagctggagaaagaacgagagaaaatccttgaatacaagcgtcaggtgcagaacttggtaaacaagtctaagaagattgtacagctgaagcctcgtaacccagactacagaagcaataaacccattattctcagagctctctgtgactacaaacaagatcagaaaatcgtgcataagggggatgagtgtatcctgaaggacaacaacgagcgcagcaagtggtacgtgacgggcccgggaggcgttgacatgcttgttccctctgtggggctgatcatccctcctccgaacccactggccgtggacctctcttgcaagattgagcagtactacgaagccatcttggctctgtggaaccagctctacatcaacatgaagagcctggtgtcctggcactactgcatgattgacatagagaagatcagggccatgacaatcgccaagctgaaaacaatgcggcaggaagattacatgaagacgatagccgaccttgagttacattaccaagagttcatcagaaatagccaaggctcagagatgtttggagatgatgacaagcggaaaatacagtctcagttcaccgatgcccagaagcattaccagaccctggtcattcagctccctggctatccccagcaccagacagtgaccacaactgaaatcactcatcatggaacctgccaagatgtcaaccataataaagtaattgaaaccaacagagaaaatgacaagcaagaaacatggatgctgatggagctgcagaagattcgcaggcagatagagcactgcgagggcaggatgactctcaaaaacctccctctagcagaccagggatcttctcaccacatcacagtgaaaattaacgagcttaagagtgtgcagaatgattcacaagcaattgctgaggttctcaaccagcttaaagatatgcttgccaacttcagaggttctgaaaagtactgctatttacagaatgaagtatttggactatttcagaaactggaaaatatcaatggtgttacagatggctacttaaatagcttatgcacagtaagggcactgctccaggctattctccaaacagaagacatgttaaaggtttatgaagccaggctcactgaggaggaaactgtctgcctggacctggataaagtggaagcttaccgctgtggactgaagaaaataaaaaatgacttgaacttgaagaagtcgttgttggccactatgaagacagaactacagaaagcccagcagatccactctcagacttcacagcagtatccactttatgatctggacttgggcaagttcggtgaaaaagtcacacagctgacagaccgctggcaaaggatagataaacagatcgactttaggttatgggacctggagaaacaaatcaagcaattgaggaattatcgtgataactatcaggctttctgcaagtggctctatgatgctaaacgccgccaggattccttagaatccatgaaatttggagattccaacacagtcatgcggtttttgaatgagcagaagaacttgcacagtgaaatatctggcaaacgagacaaatcagaggaagtacaaaaaattgctgaactttgcgccaattcaattaaggattatgagctccagctggcctcatacacctcaggactggaaactctgctgaacatacctatcaagaggaccatgattcagtccccttctggggtgattctgcaagaggctgcagatgttcatgctcggtacattgaactacttacaagatctggagactattacaggttcttaagtgagatgctgaagagtttggaagatctgaagctgaaaaataccaagatcgaagttttggaagaggagctcagactggcccgagatgccaactcggaaaactgtaataagaacaaattcctggatcagaacctgcagaaataccaggcagagtgttcccagttcaaagcgaagcttgcgagcctggaggagctgaagagacaggctgagctggatgggaagtcggctaagcaaaatctagacaagtgctacggccaaataaaagaactcaatgagaagatcacccgactgacttatgagattgaagatgaaaagagaagaagaaaatctgtggaagacagatttgaccaacagaagaatgactatgaccaactgcagaaagcaaggcaatgtgaaaaggagaaccttggttggcagaaattagagtctgagaaagccatcaaggagaaggagtacgagattgaaaggttgagggttctactgcaggaagaaggcacccggaagagagaatatgaaaatgagctggcaaaggtaagaaaccactataatgaggagatgagtaatttaaggaacaagtatgaaacagagattaacattacgaagaccaccatcaaggagatatccatgcaaaaagaggatgattccaaaaatcttagaaaccagcttgatagactttcaagggaaaatcgagatctgaaggatgaaattgtcaggctcaatgacagcatcttgcaggccactgagcagcgaaggcgagctgaagaaaacgcccttcagcaaaaggcctgtggctctgagataatgcagaagaagcagcatctggagatagaactgaagcaggtcatgcagcagcgctctgaggacaatgcccggcacaagcagtccctggaggaggctgccaagaccattcaggacaaaaataaggagatcgagagactcaaagctgagtttcaggaggaggccaagcgccgctgggaatatgaaaatgaactgagtaaggtaagaaacaattatgatgaggagatcattagcttaaaaaatcagtttgagaccgagatcaacatcaccaagaccaccatccaccagctcaccatgcagaaggaagaggataccagtggctaccgggctcagatagacaatctcacccgagaaaacaggagcttatctgaagaaataaagaggctgaagaacactctaacccagaccacagagaatctcaggagggtggaagaagacatccaacagcaaaaggccactggctctgaggtgtctcagaggaaacagcagctggaggttgagctgagacaagtcactcagatgcgaacagaggagagcgtaagatataagcaatctcttgatgatgctgccaaaaccatccaggataaaaacaaggagatagaaaggttaaaacaactgatcgacaaagaaacaaatgaccggaaatgcctggaagatgaaaacgcgagattacaaagggtccagtatgacctgcagaaagcaaacagtagtgcgacggagacaataaacaaactgaaggttcaggagcaagaactgacacgcctgaggatcgactatgaaagggtttcccaggagaggactgtgaaggaccaggatatcacgcggttccagaactctctgaaagagctgcagctgcagaagcagaaggtggaagaggagctgaatcggctgaagaggaccgcgtcagaagactcctgcaagaggaagaagctggaggaagagctggaaggcatgaggaggtcgctgaaggagcaagccatcaaaatcaccaacctgacccagcagctggagcaggcatccattgttaagaagaggagtgaggatgacctccggcagcagagggacgtgctggatggccacctgagggaaaagcagaggacccaggaagagctgaggaggctctcttctgaggtcgaggccctgaggcggcagttactccaggaacaggaaagtgtcaaacaagctcacttgaggaatgagcatttccagaaggcgatagaagataaaagcagaagcttaaatgaaagcaaaatagaaattgagaggctgcagtctctcacagagaacctgaccaaggagcacttgatgttagaagaagaactgcggaacctgaggctggagtacgatgacctgaggagaggacgaagcgaagcggacagtgataaaaatgcaaccatcttggaactaaggagccagctgcagatcagcaacaaccggaccctggaactgcaggggctgattaatgatttacagagagagagggaaaatttgagacaggaaattgagaaattccaaaagcaggctttagaggcatctaataggattcaggaatcaaagaatcagtgtactcaggtggtacaggaaagagagagccttctggtgaaaatcaaagtcctggagcaagacaaggcaaggctgcagaggctggaggatgagctgaatcgtgcaaaatcaactctagaggcagaaaccagggtgaaacagcgcctggagtgtgagaaacagcaaattcagaatgacctgaatcagtggaagactcaatattcccgcaaggaggaggctattaggaagatagaatcggaaagagaaaagagtgagagagagaagaacagtcttaggagtgagatcgaaagactccaagcagagatcaagagaattgaagagaggtgcaggcgtaagctggaggattctaccagggagacacagtcacagttagaaacagaacgctcccgatatcagagggagattgataaactcagacagcgcccatatgggtcccatcgagagacccagactgagtgtgagtggaccgttgacacctccaagctggtgtttgatgggctgaggaagaaggtgacagcaatgcagctctatgagtgtcagctgatcgacaaaacaaccttggacaaactattgaaggggaagaagtcagtggaagaagttgcttctgaaatccagccattccttcggggtgcaggatctatcgctggagcatctgcttctcctaaggaaaaatactctttggtagaggccaagagaaagaaattaatcagcccagaatccacagtcatgcttctggaggcccaggcagctacaggtggtataattgatccccatcggaatgagaagctgactgtcgacagtgccatagctcgggacctcattgacttcgatgaccgtcagcagatatatgcagcagaaaaagctatcactggttttgatgatccattttcaggcaagacagtatctgtttcagaagccatcaagaaaaatttgattgatagagaaaccggaatgcgcctgctggaagcccagattgcttcagggggtgtagtagaccctgtgaacagtgtctttttgccaaaagatgtcgccttggcccgggggctgattgatagagatttgtatcgatccctgaatgatccccgagatagtcagaaaaactttgtggatccagtcaccaaaaagaaggtcagttacgtgcagctgaaggaacggtgcagaatcgaaccacatactggtctgctcttgctttcagtacagaagagaagcatgtccttccaaggaatcagacaacctgtgaccgtcactgagctagtagattctggtatattgagaccgtccactgtcaatgaactggaatctggtcagatttcttatgacgaggttggtgagagaattaaggacttcctccagggttcaagctgcatagcaggcatatacaatgagaccacaaaacagaagcttggcatttatgaggccatgaaaattggcttagtccgacctggtactgctctggagttgctggaagcccaagcagctactggctttatagtggatcctgttagcaacttgaggttaccagtggaggaagcctacaagagaggtctggtgggcattgagttcaaagagaagctcctgtctgcagaacgagctgtcactgggtataatgatcctgaaacaggaaacatcatctctttgttccaagccatgaataaggaactcatcgaaaagggccacggtattcgcttattagaagcacagatcgcaaccggggggatcattgacccaaaggagagccatcgtttaccagttgacatagcatataagaggggctatttcaatgaggaactcagtgagattctctcagatccaagtgatgataccaaaggattttttgaccccaacactgaagaaaatcttacctatctgcaactaaaagaaagatgcattaaggatgaggaaacagggctctgtcttctgcctctgaaagaaaagaagaaacaggtgcagacatcacaaaagaataccctcaggaagcgtagagtggtcatagttgacccagaaaccaataaagaaatgtctgttcaggaggcctacaagaagggcctaattgattatgaaaccttcaaagaactgtgtgagcaggaatgtgaatgggaagaaataaccatcacgggatcagatggctccaccagggtggtcctggtagatagaaagacaggcagtcagtatgatattcaagatgctattgacaagggccttgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccggggtcccgctccggctcccgctcgggatctcgctccggatctcgctccgggtcccgcagtgggtcccggagaggaagctttgacgccacagggaattcttcctactcttattcctactcatttagcagtagttctattgggcactagtagtcagttgggagtggttgctataccttgacttcatttatatgaatttccactttattaaataatagaaaagaaaatcccggtgcttgcagtagagtgataggacattctatgcttacagaaaatatagccatgattgaaatcaaatagtaaaggctgttctggctttttatcttcttagctcatcttaaataagcagtacacttggatgcagtgcgtctgaagtgctaatcagttgtaacaatagcacaaatcgaacttaggatttgtttcttctcttctgtgtttcgatttttgatcaattctttaattttggaagcctataatacagttttctattcttggagataaaaattaaatggatcactgatattttagtcattctgcttctcatctaaatatttccatattctgtattaggagaaaattaccctcccagcaccagcccccctctcaaacccccaacccaaaaccaagcattttggaatgagtctcctttagtttcagagtgtggattgtataacccatatactcttcgatgtacttgtttggtttggtattaatttgactgtgcatgacagcggcaatcttttctttggtcaaagttttctgtttattttgcttgtcatattcgatgtactttaaggtgtctttatgaagtttgctattctggcaataaacttttagacttttgaagtgtttgtgttttaatttaatatgtttataagcatgtataaacatttagcatatttttatcataggtctaaaaatatttgtttactaaatacctgtgaagaaataccattaaaaaactatttggttctgaattcttactagaaaaaaaa

SEQ ID NO: 45 - protein, 2871 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKVRNHYNEEMSNLRNKYETEINITKTTIKEISMQKEDDSKNLRNQLDRLSRENRDLKDEIVRLNDSILQATEQRRRAEENALQQKACGSEIMQKKQHLEIELKQVMQQRSEDNARHKQSLEEAAKTIQDKNKEIERLKAEFQEEAKRRWEYENELSKVRNNYDEEIISLKNQFETEINITKTTIHQLTMQKEEDTSGYRAQIDNLTRENRSLSEEIKRLKNTLTQTTENLRRVEEDIQQQKATGSEVSQRKQQLEVELRQVTQMRTEESVRYKQSLDDAAKTIQDKNKEIERLKQLIDKETNDRKCLEDENARLQRVQYDLQKANSSATETINKLKVQEQELTRLRIDYERVSQERTVKDQDITRFQNSLKELQLQKQKVEEELNRLKRTASEDSCKRKKLEEELEGMRRSLKEQAIKITNLTQQLEQASIVKKRSEDDLRQQRDVLDGHLREKQRTQEELRRLSSEVEALRRQLLQEQESVKQAHLRNEHFQKAIEDKSRSLNESKIEIERLQSLTENLTKEHLMLEEELRNLRLEYDDLRRGRSEADSDKNATILELRSQLQISNNRTLELQGLINDLQRERENLRQEIEKFQKQALEASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 46 - DNA, 8473 bp, Homo sapiens
  source 1..8473: mol_type = genomic DNA
aagaaaccggccaggtgtggcctaggcgcccagtgccagcggggaggagactcgctccgccgccgaccaacaccaacacccagctccgacgcagctcctctgcgcccttgccgccctccgagccacagctttcctcccgctcctgcccccggcccgtcgccgtctccgcgctcgcagcggcctcgggagggcccaggtagcgagcagcgacctcgcgagccttccgcactcccgcccggttccccggccgtccgcctatccttggccccctccgctttctccgcgccggcccgcctcgcttatgcctcggcgctgagccgctctcccgattgcccgccgacatgagctgcaacggaggctcccacccgcggatcaacactctgggccgcatgatccgcgccgagtctggcccggacctgcgctacgaggtgaccagcggcggcgggggcaccagcaggatgtactattctcggcgcggcgtgatcaccgaccagaactcggacggctactgtcaaaccggcacgatgtccaggcaccagaaccagaacaccatccaggagctgctgcagaactgctccgactgcttgatgcgagcagagctcatcgtgcagcctgaattgaagtatggagatggaatacaactgactcggagtcgagaattggatgagtgttttgcccaggccaatgaccaaatggaaatcctcgacagcttgatcagagagatgcggcagatgggccagccctgtgatgcttaccagaaaaggcttcttcagctccaagagcaaatgcgagccctttataaagccatcagtgtccctcgagtccgcagggccagctccaagggtggtggaggctacacttgtcagagtggctctggctgggatgagttcaccaaacatgtcaccagtgaatgtttggggtggatgaggcagcaaagggcggagatggacatggtggcctggggtgtggacctggcctcagtggagcagcacattaacagccaccggggcatccacaactccatcggcgactatcgctggcagctggacaaaatcaaagccgacctgcgcgagaaatctgcgatctaccagttggaggaggagtatgaaaacctgctgaaagcgtcctttgagaggatggatcacctgcgacagctgcagaacatcattcaggccacgtccagggagatcatgtggatcaatgactgcgaggaggaggagctgctgtacgactggagcgacaagaacaccaacatcgctcagaaacaggaggccttctccatacgcatgagtcaactggaagttaaagaaaaagagctcaataagctgaaacaagaaagtgaccaacttgtcctcaatcagcatccagcttcagacaaaattgaggcctatatggacactctgcagacgcagtggagttggattcttcagatcaccaagtgcattgatgttcatctgaaagaaaatgctgcctactttcagttttttgaagaggcgcagtctactgaagcatacctgaaggggctccaggactccatcaggaagaagtacccctgcgacaagaacatgcccctgcagcacctgctggaacagatcaaggagctggagaaagaacgagagaaaatccttgaatacaagcgtcaggtgcagaacttggtaaacaagtctaagaagattgtacagctgaagcctcgtaacccagactacagaagcaataaacccattattctcagagctctctgtgactacaaacaagatcagaaaatcgtgcataagggggatgagtgtatcctgaaggacaacaacgagcgcagcaagtggtacgtgacgggcccgggaggcgttgacatgcttgttccctctgtggggctgatcatccctcctccgaacccactggccgtggacctctcttgcaagattgagcagtactacgaagccatcttggctctgtggaaccagctctacatcaacatgaagagcctggtgtcctggcactactgcatgattgacatagagaagatcagggccatgacaatcgccaagctgaaaacaatgcggcaggaagattacatgaagacgatagccgaccttgagttacattaccaagagttcatcagaaatagccaaggctcagagatgtttggagatgatgacaagcggaaaatacagtctcagttcaccgatgcccagaagcattaccagaccctggtcattcagctccctggctatccccagcaccagacagtgaccacaactgaaatcactcatcatggaacctgccaagatgtcaaccataataaagtaattgaaaccaacagagaaaatgacaagcaagaaacatggatgctgatggagctgcagaagattcgcaggcagatagagcactgcgagggcaggatgactctcaaaaacctccctctagcagaccagggatcttctcaccacatcacagtgaaaattaacgagcttaagagtgtgcagaatgattcacaagcaattgctgaggttctcaaccagcttaaagatatgcttgccaacttcagaggttctgaaaagtactgctatttacagaatgaagtatttggactatttcagaaactggaaaatatcaatggtgttacagatggctacttaaatagcttatgcacagtaagggcactgctccaggctattctccaaacagaagacatgttaaaggtttatgaagccaggctcactgaggaggaaactgtctgcctggacctggataaagtggaagcttaccgctgtggactgaagaaaataaaaaatgacttgaacttgaagaagtcgttgttggccactatgaagacagaactacagaaagcccagcagatccactctcagacttcacagcagtatccactttatgatctggacttgggcaagttcggtgaaaaagtcacacagctgacagaccgctggcaaaggatagataaacagatcgactttaggttatgggacctggagaaacaaatcaagcaattgaggaattatcgtgataactatcaggctttctgcaagtggctctatgatgctaaacgccgccaggattccttagaatccatgaaatttggagattccaacacagtcatgcggtttttgaatgagcagaagaacttgcacagtgaaatatctggcaaacgagacaaatcagaggaagtacaaaaaattgctgaactttgcgccaattcaattaaggattatgagctccagctggcctcatacacctcaggactggaaactctgctgaacatacctatcaagaggaccatgattcagtccccttctggggtgattctgcaagaggctgcagatgttcatgctcggtacattgaactacttacaagatctggagactattacaggttcttaagtgagatgctgaagagtttggaagatctgaagctgaaaaataccaagatcgaagttttggaagaggagctcagactggcccgagatgccaactcggaaaactgtaataagaacaaattcctggatcagaacctgcagaaataccaggcagagtgttcccagttcaaagcgaagcttgcgagcctggaggagctgaagagacaggctgagctggatgggaagtcggctaagcaaaatctagacaagtgctacggccaaataaaagaactcaatgagaagatcacccgactgacttatgagattgaagatgaaaagagaagaagaaaatctgtggaagacagatttgaccaacagaagaatgactatgaccaactgcagaaagcaaggcaatgtgaaaaggagaaccttggttggcagaaattagagtctgagaaagccatcaaggagaaggagtacgagattgaaaggttgagggttctactgcaggaagaaggcacccggaagagagaatatgaaaatgagctggcaaaggtaagaaaccactataatgaggagatgagtaatttaaggaacaagtatgaaacagagattaacattacgaagaccaccatcaaggagatatccatgcaaaaagaggatgattccaaaaatcttagaaaccagcttgatagactttcaagggaaaatcgagatctgaaggatgaaattgtcaggctcaatgacagcatcttgcaggccactgagcagcgaaggcgagctgaagaaaacgcccttcagcaaaaggcctgtggctctgagataatgcagaagaagcagcatctggagatagaactgaagcaggtcatgcagcagcgctctgaggacaatgcccggcacaagcagtccctggaggaggctgccaagaccattcaggacaaaaataaggagatcgagagactcaaagctgagtttcaggaggaggccaagcgccgctgggaatatgaaaatgaactgagtaaggcatctaataggattcaggaatcaaagaatcagtgtactcaggtggtacaggaaagagagagccttctggtgaaaatcaaagtcctggagcaagacaaggcaaggctgcagaggctggaggatgagctgaatcgtgcaaaatcaactctagaggcagaaaccagggtgaaacagcgcctggagtgtgagaaacagcaaattcagaatgacctgaatcagtggaagactcaatattcccgcaaggaggaggctattaggaagatagaatcggaaagagaaaagagtgagagagagaagaacagtcttaggagtgagatcgaaagactccaagcagagatcaagagaattgaagagaggtgcaggcgtaagctggaggattctaccagggagacacagtcacagttagaaacagaacgctcccgatatcagagggagattgataaactcagacagcgcccatatgggtcccatcgagagacccagactgagtgtgagtggaccgttgacacctccaagctggtgtttgatgggctgaggaagaaggtgacagcaatgcagctctatgagtgtcagctgatcgacaaaacaaccttggacaaactattgaaggggaagaagtcagtggaagaagttgcttctgaaatccagccattccttcggggtgcaggatctatcgctggagcatctgcttctcctaaggaaaaatactctttggtagaggccaagagaaagaaattaatcagcccagaatccacagtcatgcttctggaggcccaggcagctacaggtggtataattgatccccatcggaatgagaagctgactgtcgacagtgccatagctcgggacctcattgacttcgatgaccgtcagcagatatatgcagcagaaaaagctatcactggttttgatgatccattttcaggcaagacagtatctgtttcagaagccatcaagaaaaatttgattgatagagaaaccggaatgcgcctgctggaagcccagattgcttcagggggtgtagtagaccctgtgaacagtgtctttttgccaaaagatgtcgccttggcccgggggctgattgatagagatttgtatcgatccctgaatgatccccgagatagtcagaaaaactttgtggatccagtcaccaaaaagaaggtcagttacgtgcagctgaaggaacggtgcagaatcgaaccacatactggtctgctcttgctttcagtacagaagagaagcatgtccttccaaggaatcagacaacctgtgaccgtcactgagctagtagattctggtatattgagaccgtccactgtcaatgaactggaatctggtcagatttcttatgacgaggttggtgagagaattaaggacttcctccagggttcaagctgcatagcaggcatatacaatgagaccacaaaacagaagcttggcatttatgaggccatgaaaattggcttagtccgacctggtactgctctggagttgctggaagcccaagcagctactggctttatagtggatcctgttagcaacttgaggttaccagtggaggaagcctacaagagaggtctggtgggcattgagttcaaagagaagctcctgtctgcagaacgagctgtcactgggtataatgatcctgaaacaggaaacatcatctctttgttccaagccatgaataaggaactcatcgaaaagggccacggtattcgcttattagaagcacagatcgcaaccggggggatcattgacccaaaggagagccatcgtttaccagttgacatagcatataagaggggctatttcaatgaggaactcagtgagattctctcagatccaagtgatgataccaaaggattttttgaccccaacactgaagaaaatcttacctatctgcaactaaaagaaagatgcattaaggatgaggaaacagggctctgtcttctgcctctgaaagaaaagaagaaacaggtgcagacatcacaaaagaataccctcaggaagcgtagagtggtcatagttgacccagaaaccaataaagaaatgtctgttcaggaggcctacaagaagggcctaattgattatgaaaccttcaaagaactgtgtgagcaggaatgtgaatgggaagaaataaccatcacgggatcagatggctccaccagggtggtcctggtagatagaaagacaggcagtcagtatgatattcaagatgctattgacaagggccttgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccggggtcccgctccggctcccgctcgggatctcgctccggatctcgctccgggtcccgcagtgggtcccggagaggaagctttgacgccacagggaattcttcctactcttattcctactcatttagcagtagttctattgggcactagtagtcagttgggagtggttgctataccttgacttcatttatatgaatttccactttattaaataatagaaaagaaaatcccggtgcttgcagtagagtgataggacattctatgcttacagaaaatatagccatgattgaaatcaaatagtaaaggctgttctggctttttatcttcttagctcatcttaaataagcagtacacttggatgcagtgcgtctgaagtgctaatcagttgtaacaatagcacaaatcgaacttaggatttgtttcttctcttctgtgtttcgatttttgatcaattctttaattttggaagcctataatacagttttctattcttggagataaaaattaaatggatcactgatattttagtcattctgcttctcatctaaatatttccatattctgtattaggagaaaattaccctcccagcaccagcccccctctcaaacccccaacccaaaaccaagcattttggaatgagtctcctttagtttcagagtgtggattgtataacccatatactcttcgatgtacttgtttggtttggtattaatttgactgtgcatgacagcggcaatcttttctttggtcaaagttttctgtttattttgcttgtcatattcgatgtactttaaggtgtctttatgaagtttgctattctggcaataaacttttagacttttgaagtgtttgtgttttaatttaatatgtttataagcatgtataaacatttagcatatttttatcataggtctaaaaatatttgtttactaaatacctgtgaagaaataccattaaaaaactatttggttctgaattcttactagaaaaaaaa

SEQ ID NO: 47 - protein, 2428 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKVRNHYNEEMSNLRNKYETEINITKTTIKEISMQKEDDSKNLRNQLDRLSRENRDLKDEIVRLNDSILQATEQRRRAEENALQQKACGSEIMQKKQHLEIELKQVMQQRSEDNARHKQSLEEAAKTIQDKNKEIERLKAEFQEEAKRRWEYENELSKASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 48 - DNA, 8768 bp, Homo sapiens
  source 1..8768: mol_type = genomic DNA
gcggccgcactagtaccccggagcccatgggcgcgccgagccgggcgcgggggcgctgaacggcggagcgggagcggccggaggagccatggactgcagcctcgtgcggacgctcgtgcacagatactgtgcaggagaagagaattgggtggacagcaggaccatctacgtgggacacagggagccacctccgggcgcagaggcctacatcccacagagatacccagacaacaggatcgtctcgtccaagtacacattttggaactttatacccaagaatttatttgaacaattcagaagagtagccaacttttatttccttatcatatttctggtgcagttgattattgatacacccacaagtccagtgacaagcggacttccactcttctttgtcattactgtgacggctatcaaacagggttatgaagactggcttcgacataaagcagacaatgccatgaaccagtgtcctgttcatttcattcagcacggcaagctcgttcggaaacaaagtcgaaagctgcgagttggggacattgtcatggttaaggaggacgagacctttccctgcgacttgatcttcctttccagcaaccggggagatgggacgtgccacgtcaccaccgccagcttggatggagaatccagccataaaacgcattacgcggtccaggacaccaaaggcttccacacagaggaggatatcggcggacttcacgccaccatcgagtgtgagcagccccagcccgacctctacaagttcgtgggtcgcatcaacgtttacagtgacctgaatgaccccgtggtgaggcccttaggatcggaaaacctgctgcttagaggagctacactgaagaacactgagaaaatctttggtgtggctatttacacgggaatggaaaccaagatggcattaaattatcaatcaaaatctcagaagcgatctgccgtggaaaaatcgatgaatgcgttcctcattgtgtatctctgcattctgatcagcaaagccctgataaacactgtgctgaaatacatgtggcagagtgagccctttcgggatgagccgtggtataatcagaaaacggagtcggaaaggcagaggaatctgttcctcaaggcattcacggacttcctggccttcatggtcctctttaactacatcatccctgtgtccatgtacgtcacggtcgagatgcagaagttcctcggctcttacttcatcacctgggacgaagacatgtttgacgaggagactggcgaggggcctctggtgaacacgtcggacctcaatgaagagctgggacaggtggagtacatcttcacagacaagaccggcaccctcacggaaaacaacatggagttcaaggagtgctgcatcgaaggccatgtctacgtgccccacgtcatctgcaacgggcaggtcctcccagagtcgtcaggaatcgacatgattgactcgtcccccagcgtcaacgggagggagcgcgaggagctgtttttccgggccctctgtctctgccacaccgtccaggtgaaagacgatgacagcgtagacggccccaggaaatcgccggacggggggaaatcctgtgtgtacatctcatcctcgcccgacgaggtggcgctggtcgaaggtgtccagagacttggctttacctacctaaggctgaaggacaattacatggagatattaaacagggagaaccacatcgaaaggtttgaattgctggaaattttgagttttgactcagtcagaaggagaatgagtgtaattgtaaaatctgctacaggagaaatttatctgttttgcaaaggagcagattcttcgatattcccccgagtgatagaaggcaaagttgaccagatccgagccagagtggagcgtaacgcagtggaggggctccgaactttgtgtgttgcttataaaaggctgatccaagaagaatatgaaggcatttgtaagctgctgcaggctgccaaagtggcccttcaagatcgagagaaaaagttagcagaagcctatgagcaaatagagaaagatcttactctgcttggtgctacagctgttgaggaccggctgcaggagaaagctgcagacaccatcgaggccctgcagaaggccgggatcaaagtctgggttctcacgggagacaagatggagacggccgcggccacgtgctacgcctgcaagctcttccgcaggaacacgcagctgctggagctgaccaccaagaggatcgaggagcagagcctgcacgacgtcctgttcgagctgagcaagacggtcctgcgccacagcgggagcctgaccagagacaacctgtccggactttcagcagatatgcaggactacggtttaattatcgacggagctgcactgtctctgataatgaagcctcgagaagacgggagttccggcaactacagggagctcttcctggaaatctgccggagctgcagcgcggtgctctgctgccgcatggcgcccttgcagaaggctcagattgttaaattaatcaaattttcaaaagagcacccaatcacgttagcaattggcgatggtgcaaatgatgtcagcatgattctggaagcgcacgtgggcataggtgtcatcggcaaggaaggccgccaggctgccaggaacagcgactatgcaatcccaaagtttaagcatttgaagaagatgctgcttgttcacgggcatttttattacattaggatctctgagctcgtgcagtacttcttctataagaacgtctgcttcatcttccctcagtttttataccagttcttctgtgggttttcacaacagactttgtacgacaccgcgtatctgaccctctacaacatcagcttcacctccctccccatcctcctgtacagcctcatggagcagcatgttggcattgacgtgctcaagagagacccgaccctgtacagggacgtcgccaagaatgccctgctgcgctggcgcgtgttcatctactggacgctcctgggactgtttgacgcactggtgttcttctttggtgcttatttcgtgtttgaaaatacaactgtgacaagcaacgggcagatatttggaaactggacgtttggaacgctggtattcaccgtgatggtgttcacagttacactaaagcttgcattggacacacactactggacttggatcaaccattttgtcatctgggggtcgctgctgttctacgttgtcttttcgcttctctggggaggagtgatctggccgttcctcaactaccagaggatgtactacgtgttcatccagatgctgtccagcgggcccgcctggctggccatcgtgctgctggtgaccatcagcctccttcccgacgtcctcaagaaagtcctgtgccggcagctgtggccaacagcaacagagagagtccagaatgggtgcgcacagcctcgggaccgcgactcagaattcacccctcttgcctctctgcagagcccaggctaccagagcacctgtccctcggccgcctggtacagctcccactctcagcaggtgacactcgcggcctggaaggagaaggtgtccacggagcccccacccatcctcggcggttcccatcaccactgcagttccatcccaagtcacagctgccctaggtcccgtgtgggaatgctcgtgtgatggatggtcctaagcctgtggagactgtgcacgtgcctcttcctggcccccagcaggcaaggaggggggtcacaggccttgccctcgagcatggcaccctggccgcctggacccagcactgtggttgttgagccacaccagtggcctctgggcattcggctcaacgcaggagggacattctgctggcccaccctgcgcgctgtcatgcagaggccattcccccaggcctgtgtcttcacccacctgccatcattggcctttgctgtcactgggagagaagagccgtccagggacccatggtggcccacatgtggatgccacatgctgctgtttcctgcttgcccggccaccacccatgccctccatagggtgaggtggagccatggtggtgcgtcctttactcaacaaccctccaatccggatgctgtgggaagggccgggtcactcggataccatcatccctgcggatgcaccgccgtaccctgctcatctgggagtggtttccctgcggttacgtccaagcccgcctgccctgtgtgttggggctggctgagtttcggtctccccatcaccggccgcctcgtggagaaggcagtgccacgtgggaggacaaggccacgccggcagcttccagccctgccgcagaagtgccaggatgtccatcagccactcgccagggcacggagccgtcagtccactgttacgggagaatgttgatttcgcgggtgcgagggccgggagacagatacttggctgtgatgagcagacatcctctgtccccgtggaggggtcaacaccaaggtggtgttcgtgcaccagaacctgtctcgggctgacgggggtggcacacaggacacgggtggatcccaacaggcagcaccgcacctctgcccgcctcccgcactgcagctccgcccgccgggctctgcgtccccacgtcccctcgtcccatccccacgtcccctcatcccgtcacctcgtccccacatccccttgccccgtcacctcgtcctcatgtccccttgtcctgtcacctcgtccccacgtcccctcgtctcctcatccccacgtcctctcgtccccttgtcccgtccccacataccctcgtccccatgtccccacgcagggctctccttcgtcttaggatctgtccagcgctgctctgggtgggttagcaaccccagggctgctgtgataggaagtccctgttgttctccgtactggcatttctatttctagaaataatatttgacatagccttaatggtccttaaagaagacatttcagtgtgagattcagacttcagacgctgaaactgctgcctttcaggaaagcaccaccaacgctggaggaggagccggccctcacgcccgccccgcgccacgctgtggaacggggctccggcaagtgaaacccagagggtgtttccgaggtgctcgacagtaggtatttttggaagctcagatttcaccatttgattgtataatcttttacctataaaatatttatttgaagtagagggtaaatcagcggtaagaacagtgaacacagtggttgggataaaataaggtgacaaacatcacaccaaagatgagggtagcgagcaactggcttgagcagacagaacggggaagactccactctgtcccgaggggccagccgcaggcgtccccagggccaccctgccctgaggtccttgtgtggccgccctggcttggcagccctgcccacgctgcccccgcaaacaatggtgtgtgcgtttttacagccctttttaggaacccaatatgggcataaatgtaacacctgtagcgggggcagattctctgtatgttcagttaacaaattatttgtaatgtatttttttagaaatcttaaaattgcctttgcactgaagtattttcatagctgtttatatctcttttattcatttatttaacatactgtctaattttaaaaataggtttttaaagctttcatttttaagtttatgaaattttggccactttacatttagattctggtgagagttttgactgaatgttccaatctctgatgaatgcgaattttcagatttgattttattctctacacacacctcttcttttcttggtatttctggtggcagtgattagttgaacagcacatttaaggcacgataatttgctacactttttctttacaatttgttgcaatttcatctgctttctatgtttcattgttaattgccatccttcagccttaaaaatagaagattctcacgtgaaggtttagtaagttgggtcccagctctgcctgtgtggagatagtcaccatgtacctctgacaacaagttttagtgtgaaagtcactaaacttttacacactcccaaacgtctttttaaaaattgcttgggaaattattaaatgaatgtgcctgatgatttgaaatagacaaggggcacgagataaaaaagaaaaggatgagaagatcctcagtgaatgacgttgcagggtcttcatgcaattttccacctcgcagtagttagtatttacttgccttaaactaactttgaagcaagtaatgtcaactttgagcactttgttgagttttgaaaaatcttatttgttgctgcacaggttaataaattatcaatttgtaattcagcatgttggtcagagacacggtcactgattcacacccagtccctgccacagaccgtctcagacacgcacagtgggcctgctgcatgattcacacccagtccctgccacagaccgtctcagacacgcacagtgggcctgctgcatgattcacacccagtccctgccacagaccgtctcagacacgcacagtgggcctgctgcatgcgtgttacctggcttttggctccacgctcactcatagccatgtccacatgggggcttgcacacaggatcactcacatatgtacatgtacccaccacaaacgtgcaagctcctgcacacatgcatgcacacaaacgtgtacacaagtgtgagctcctacacgcatacacacacacacgtgtacatgcaccaaagcatgtgtgacctacagacatgcagaacatgcacgtgtacacataccacagacacgcgtgtgcatgctcctacacaatacatatgcacatatcatgaacagcgtaagttcctacacacggacgtgtgatacacacatgcatgtacaggtaagcacacatgtacaagctcctacaggcttgctctcacacacgtgtatgcacagcagagagacgtatgagcttctactgcacacatgcacacacacacgcacacgtacattcactacaaacgtgcagcctcctgcacacgtgcacattcatgtgtacaccacaaatgagttcccagacgtgtaaacacacgtgcacacatcgtacacatgtgagctcccacacgtacacacagatgcacatggacacaccccaaacacgcacaggctcctacacacatgcacacacgtgtacaccacaaacgagctcccagacatgtaaacacacgtctcccacacgtgagctcccacacgtacacatgcacatgtacgcaccacaaacacatgcgcaggctcctgcaggcgtgaatacacacatgcacacacatatacacacatgtgccacaaacaagtgcacactgtcctggtgtcctgcactgcatcctgcctccttgctgaggggcccctgtgagaggcctctggatgggcatgggaagatgggctccctggcccccagcccatgcctccctgggatgaagagtccccctcctggcagaatgtctgggctttgcagagcaggccccgggggtgaagtcgcagcttcacttacaccagctgctctgtgagcaaggcttggtgccctggacaaggcccttcccctttagggaggtccagcctcgcaagctgaaacctcccctcggctcagccctataccaggcggccacagcaggactggccacacccacgccgcacctcatccgtgcacgcgtcggagcacggccagccttccgccacgagccagctgggaagggccgcggccgcctaaagccccagtcaacccagcctgtgtctgagcagacagggcgaacaagcaggccacaccgtctcgagggaggaggccagatgcggccagcgtctccaacagggtgaccatccgctcggcttgctgagcgtttaaacaaatgtttagacaggctgtggggactcccctgagttgagccttggccaggggtccggtgctgtcgcgggaaacctccagccttgttcttcaaaccactcagctcatgtgttttgcactgactagtactgaataatacaaccactcttatttaatgttagtattatttatttgacaactcagtgtctaacagcttgatatgcaggtccttgcatcctacatttctttaggaagttacccatttgtaactttaaaaacaggaaaaatatcagttggcaaatgcaatcttttttttttttaagctaaaggtgggtgaactggaatgaaaatctttctgatgttgtgtctataagcagccttgatgggatatgttagaagtgtcatgaaagtgtgattctacttttgcagaaaaatctaaagatcaatttatatagctttattttttactttatcaaagtatacagaattttaatatgcatatattgtgtctgacttaaaattataatgtctgcgtcaccatttaaaatgtctgttcattatgtaatgtaataaaagaaggtcttcaaaaatgtatttaacatgaatggtatccatagttgtcatcatcataaatactggagtttatttttaaattattaaacatagtaggtgcattaacataaatcagtctccacacagtaacatttaactgataattcattaatcagctttgaaaaattaaattgttaattaaaccaatctaacatttcagtaaagtttattttgtatgcttctgtttttaacttttatttctgtagataaactgactggataatattatattggacttttctctagattatctaagcaggagacctgaatctgcttgcaataaagaataaaagtctgcttcagtttctttataaagaaactcacacaa

SEQ ID NO: 49 - protein, 1191 aa, Homo sapiens
MDCSLVRTLVHRYCAGEENWVDSRTIYVGHREPPPGAEAYIPQRYPDNRIVSSKYTFWNFIPKNLFEQFRRVANFYFLIIFLVQLIIDTPTSPVTSGLPLFFVITVTAIKQGYEDWLRHKADNAMNQCPVHFIQHGKLVRKQSRKLRVGDIVMVKEDETFPCDLIFLSSNRGDGTCHVTTASLDGESSHKTHYAVQDTKGFHTEEDIGGLHATIECEQPQPDLYKFVGRINVYSDLNDPVVRPLGSENLLLRGATLKNTEKIFGVAIYTGMETKMALNYQSKSQKRSAVEKSMNAFLIVYLCILISKALINTVLKYMWQSEPFRDEPWYNQKTESERQRNLFLKAFTDFLAFMVLFNYIIPVSMYVTVEMQKFLGSYFITWDEDMFDEETGEGPLVNTSDLNEELGQVEYIFTDKTGTLTENNMEFKECCIEGHVYVPHVICNGQVLPESSGIDMIDSSPSVNGREREELFFRALCLCHTVQVKDDDSVDGPRKSPDGGKSCVYISSSPDEVALVEGVQRLGFTYLRLKDNYMEILNRENHIERFELLEILSFDSVRRRMSVIVKSATGEIYLFCKGADSSIFPRVIEGKVDQIRARVERNAVEGLRTLCVAYKRLIQEEYEGICKLLQAAKVALQDREKKLAEAYEQIEKDLTLLGATAVEDRLQEKAADTIEALQKAGIKVWVLTGDKMETAAATCYACKLFRRNTQLLELTTKRIEEQSLHDVLFELSKTVLRHSGSLTRDNLSGLSADMQDYGLIIDGAALSLIMKPREDGSSGNYRELFLEICRSCSAVLCCRMAPLQKAQIVKLIKFSKEHPITLAIGDGANDVSMILEAHVGIGVIGKEGRQAARNSDYAIPKFKHLKKMLLVHGHFYYIRISELVQYFFYKNVCFIFPQFLYQFFCGFSQQTLYDTAYLTLYNISFTSLPILLYSLMEQHVGIDVLKRDPTLYRDVAKNALLRWRVFIYWTLLGLFDALVFFFGAYFVFENTTVTSNGQIFGNWTFGTLVFTVMVFTVTLKLALDTHYWTWINHFVIWGSLLFYVVFSLLWGGVIWPFLNYQRMYYVFIQMLSSGPAWLAIVLLVTISLLPDVLKKVLCRQLWPTATERVQNGCAQPRDRDSEFTPLASLQSPGYQSTCPSAAWYSSHSQQVTLAAWKEKVSTEPPPILGGSHHHCSSIPSHSCPRSRVGMLV

SEQ ID NO: 50 - DNA, 4673 bp, Homo sapiens
  source 1..4673: mol_type = genomic DNA
tttccgcagttaggggctgctatttcaacgcagggagataaaaagaaaaaaacacttgctcttctaccccgctaaaaacactcatcctagggagcacgccagcatttgcagcgttcggggcagggccactcggcctgcggccgttgcactggctggaagctggcaggcgatcacggttgattggctcgggtgcggtccaagggcagcaacgccttcggcgggccgcctagggtgattggctgctgcagcccaccccctagccggtttggtgggcggcgaagcctggattggtggagctaagagctggctcagtttcagcgctggctcttcgtgcatggcagagatggcgactgcgactcggctgctggggtggcgtgtggcgagctggaggctgcggccgccgcttgccggcttcgtttcccagcgggcccactcgcttttgcccgtggacgatgcaatcaatgggctaagcgaggagcagaggcagcttcgtcagaccatggctaagttccttcaggagcacctggcccccaaggcccaggagatcgatcgcagcaatgagttcaagaacctgcgagaattttggaagcagctggggaacctgggcgtattgggcatcacagcccctgttcagtatggcggctccggcctgggctacctggagcatgtgctggtgatggaggagatatcccgagcttccggagcagtggggctcagttacggtgcccactccaacctctgcatcaaccagcttgtacgcaatgggaatgaggcccagaaagagaagtatctcccgaagctgatcagtggtgagtacatcggagccctggccatgagtgagcccaatgcaggctctgatgttgtctctatgaagctcaaagcggaaaagaaaggaaatcactacatcctgaatggcaacaagttctggatcactaatggccctgatgctgacgtcctgattgtctatgccaagacagatctggctgctgtgccagcttctcggggcatcacagccttcattgtggagaagggtatgcctggctttagcacctctaagaagctggacaagctggggatgaggggctctaacacctgtgagctaatctttgaagactgcaagattcctgctgccaacatcctgggccatgagaataagggtgtctacgtgctgatgagtgggctggacctggagcggctggtgctggccggggggcctcttgggctcatgcaagcggtcctggaccacaccattccctacctgcacgtgagggaagcctttggccagaagatcggccacttccagttgatgcaggggaagatggctgacatgtacacccgcctcatggcgtgtcggcagtatgtctacaatgtcgccaaggcctgcgatgagggccattgcactgctaaggactgtgcaggtgtgattctttactcagctgagtgtgccacacaggtagccctggacggcattcagtgttttggtggcaatggctacatcaatgactttcccatgggccgctttcttcgagatgccaagctgtatgagataggggctgggaccagcgaggtgaggcggctggtcatcggcagagccttcaatgcagactttcactagtcctgagacccttcgcccccttttcctgcacctagtggcctttcttgggaagtagagatgtggcggctttcccaccctgcccacagcaggccctcctgcccagctgctcttgtcagccctctggcctctggatgaggttgagttctccacaacagctcccaagcatcatgggcctcgcagccgggcctgtgccacggctagtgttgtgtgatttaaaatggactcagcaggaagcatattgtctggggattgttgggacaggttttggtgactctgtgcccttgctctctaacttctgagcccacctcccagggtaggcacctgggggcatgcaggtgcccacctcccagggtaggcacctgggggcatgcaggtacccacctctttctcttgggtgaggctctggcaaggagatctctctgctcaagcacagcagaatcatggcccctctccatgaattggaacttggtacaggttaagtatccctaatcctgaaatctgaaacacttgtggttccaagcattttggataaggcaaattcaactttcagtctcttttctgggggaaaaaaataataaacctagcctagccaggcgtggtggctcatgcttgtaatcccagcacttcaggaggctgagatgggtggatcacctgaggtcaggagttcaagaccagcctggccaacatgtggaaacctcgcctcaactaaaaatagaaaaaaattagttgggcatggtggtgggcacctgtaatcccagctacttcaggaggctgaggcaggagaattacttgaacccaggaggcggacgttgcagtgagccgagcttgtgccattgcactccagcctgggcgacaagagcaaaactcttcaaaaaacaaaacaaaacaaaaaaaccctggcccttgtttcttccagtttctagaggtatcagctcctagcagcttatgaacacatatgcttgcttggccaggcaaggtggtgtgtgcctgtaatcccagcactttgggaggccaaggcaggtggatcacttgcagtcaggagttcaagaccagcctgtccaacgtggtgaaaccccatctctactaaaaatacaaaaattagccaggggtggtggtgcacgtctgtaatcccagctactcaggaggctgaggcaggagaatcacttgaacccgggaggtggaggttgcaatgagccaatatgacaccgctgcagtccagcctgggccatagagtgagactctgtctcaaaaaaggaaagaaaaataggctgggcacagtgactcatgcctgtaatcccaacactttgggaggccgaggcaggtggatcacgaggtcaggagttcaagaccagcctggccaagatggtaaaacctcgtctctactaaaaatacaaaaattagccaggtgtggtggcaggctcctgtaatcccagctactcaggaggctgaggcagagaattgcttgaacccgggaggcagagtttgcagtgagccaagatcacaccactgcactccagcttggacgacagagcgagactctgtctcaaaaaataataggccaggcatggtggctcaacgtctgtaatcccagcactttgggaggccgaggcgggcagatcacaaggtcaggagttcgagaccagcctgacgaccaacatggtgaaacctcgtctctactaaaaatacaaaaattagccaggcctggtggcacgcgcctgtaatcccagttacacagaagactgaggcaggagaatcgcttgaacgcaggaggcagaggttgcaggagctgagatcgcgccattgcactccagcctgggcaacagagtgagactctgtctcaaaaaataataataaaataaatgaacacacatgctgctgagtccgcagggggggcagagcagaggacagcgtgcttttgtgtactgttggaagactggctcctcctgtacagcacctctgagcccttgtgcaccgccctgccacgggcaccatccagtcctggccgtgtgaccacccacagctgactgggcagcaggcacaggccctacccgagcaggccggagttggctcgcatgactccagctgaggctgcctgtgtacatttctccagataccctatggctaattttgttataactgcacagtggctgctgccattttgtattaaatatattgtgaaacaaacctatctggggagaagcaatctacttgccgctgcttcctgtctggatccagcttgtgtccttggagagtggctggcccaggtcctattcctgtcctccagcccgttctttcatgagggacaggaaggtaaaatcagcccttaggagagaggtctcagcctccctttcccagatctcccagtgagttttaaaggaagcagggagcccagagtgctaagttcttacagccagaaggaagcttatagatttctgaaaaccgcccctttgtttttaaaaagatcaacacaatttgactttctcaaggtcaaaacgaactagaatccagatctgctcatggcaaaaatgggggtgttctgagaattccagctttgggccgcactgtacagcagtctggatagagtgtgatctgagaagggaatgggtctgggttgttccaccccttccgagttccaaaaagagggaactggttttcttggttctcagcccagcagcacctatcctggctcttggtcctggcctgcagccaagtgctgttcctagcctgaggcttgagacaggtggggttggctcctcaccaaccccagttccgtcccatcctgagggcaagatcctgggctcataggcagtccctttcacttccttgtcttgctccctgctatgttggagatgaatgtgactaaaagggccatcttgctggcttaatgtgtggctggagagaccagcctggagacaatgtggcaaaatggggcgcttcatccagtctgtctaagccctgtcgacttggggaggtgatttctttcctggttctatatgtgaagcaaaataaatgttttaaaattaaaagcaaaaaaaacaaaatgaaccatgaaaaaaaa

SEQ ID NO: 51 - protein, 426 aa, Homo sapiens
MAEMATATRLLGWRVASWRLRPPLAGFVSQRAHSLLPVDDAINGLSEEQRQLRQTMAKFLQEHLAPKAQEIDRSNEFKNLREFWKQLGNLGVLGITAPVQYGGSGLGYLEHVLVMEEISRASGAVGLSYGAHSNLCINQLVRNGNEAQKEKYLPKLISGEYIGALAMSEPNAGSDVVSMKLKAEKKGNHYILNGNKFWITNGPDADVLIVYAKTDLAAVPASRGITAFIVEKGMPGFSTSKKLDKLGMRGSNTCELIFEDCKIPAANILGHENKGVYVLMSGLDLERLVLAGGPLGLMQAVLDHTIPYLHVREAFGQKIGHFQLMQGKMADMYTRLMACRQYVYNVAKACDEGHCTAKDCAGVILYSAECATQVALDGIQCFGGNGYINDFPMGRFLRDAKLYEIGAGTSEVRRLVIGRAFNADFH

SEQ ID NO: 52 - protein, 268 aa, Homo sapiens
MSGKHYKGPEVSCCIKYFIFGFNVIFWFLGITFLGIGLWAWNEKGVLSNISSITDLGGFDPVWLFLVVGGVMFILGFAGCIGALRENTFLLKFFSVFLGIIFFLELTAGVLAFVFKDWIKDQLYFFINNNIRAYRDDIDLQNLIDFTQEYWQCCGAFGADDWNLNIYFNCTDSNASRERCGVPFSCCTKDPAEDVINTQCGYDARQKPEVDQQIVIYTKGCVPQFEKWLQDNLTIVAGIFIGIALLQIFGICLAQNLVSDIEAVRASW

SEQ ID NO: 53 - DNA, 3583 bp, Homo sapiens
  source 1..3583: mol_type = genomic DNA
ctgggccccagcgaggcggtggggcggggcggggcggggcggggcgcgcagcaggagcgagtggggccgcccgccgggccgcggacactgtcgcccggcgcccaggttcccaacaaggctacgcagaagaacccccttgactgaagcaatggaggggggtccagctgtctgctgccaggatcctcgggcagagctggtagaacgggtggcagccatcgatgtgactcacttggaggaggcagatggtggcccagagcctactagaaacggtgtggaccccccaccacgggccagagctgcctctgtgatccctggcagtacttcaagactgctcccagcccggcctagcctctcagccaggaagctttccctacaggagcggccagcaggaagctatctggaggcgcaggctgggccttatgccacggggcctgccagccacatctccccccgggcctggcggaggcccaccatcgagtcccaccacgtggccatctcagatgcagaggactgcgtgcagctgaaccagtacaagctgcagagtgagattggcaagggtgcctacggtgtggtgaggctggcctacaacgaaagtgaagacagacactatgcaatgaaagtcctttccaaaaagaagttactgaagcagtatggctttccacgtcgccctcccccgagagggtcccaggctgcccagggaggaccagccaagcagctgctgcccctggagcgggtgtaccaggagattgccatcctgaagaagctggaccacgtgaatgtggtcaaactgatcgaggtcctggatgacccagctgaggacaacctctatttggtgtttgacctcctgagaaaggggcccgtcatggaagtgccctgtgacaagcccttctcggaggagcaagctcgcctctacctgcgggacgtcatcctgggcctcgagtacttgcactgccagaagatcgtccacagggacatcaagccatccaacctgctcctgggggatgatgggcacgtgaagatcgccgactttggcgtcagcaaccagtttgaggggaacgacgctcagctgtccagcacggcgggaaccccagcattcatggcccccgaggccatttctgattccggccagagcttcagtgggaaggccttggatgtatgggccactggcgtcacgttgtactgctttgtctatgggaagtgcccattcatcgacgatttcatcctggccctccacaggaagatcaagaatgagcccgtggtgtttcctgaggagccagaaatcagcgaggagctcaaggacctgatcctgaagatgttagacaagaatcccgagacgagaattggggtgccagacatcaagttgcacccttgggtgaccaagaacggggaggagccccttccttcggaggaggagcactgcagcgtggtggaggtgacagaggaggaggttaagaactcagtcaggctcatccccagctggaccacggtgatcctggtgaagtccatgctgaggaagcgttcctttgggaacccgtttgagccccaagcacggagggaagagcgatccatgtctgctccaggaaacctactggtgaaagaagggtttggtgaagggggcaagagcccagagctccccggcgtccaggaagacgaggctgcatcctgagcccctgcatgcacccagggccacccggcagcacactcatcccgcgcctccagaggcccacccctcatgcaacagccgcccccgcaggcagggggctggggactgcagccccactcccgcccctcccccatcgtgctgcatgacctccacgcacgcacgtccagggacagactggaatgtatgtcatttggggtcttgggggcagggctcccacgaggccatcctcctcttcttggacctccttggcctgacccattctgtggggaaaccgggtgcccatggagcctcagaaatgccacccggctggttggcatggcctggggcaggaggcagaggcaggagaccaagatggcaggtggaggccaggcttaccacaacggaagagacctcccgctggggccgggcaggcctggctcagctgccacaggcatatggtggagaggggggtaccctgcccaccttggggtggtggcaccagagctcttgtctattcagacgctggtatgggggctcggacccctcactggggacagggccagtgttggagaattctgattccttttttgttgtcttttacttttgtttttaacctgggggttcggggagaggccctgcttgggaacatctcacgagctttcctacatcttccgtggttcccagcacagcccaagattatttggcagccaagtggatggaactaactttcctggactgtgtttcgcattcggcgttatctggaaagtggactgaacggaatcaagctctgagcagaggcctgaagcggaagcaccacatcgtccctgcccatctcactctctcccttgatgatgcccctagagctgaggctggagaagacaccagggctgactttgaccgagggccatggacgcgacaggcctgtggccctgcgcatgctgaaataactggaacccagcctctcctcctacaccggcctacccatctgggcccaagagctgcactcacactcctacaacgaaggacaaactgtccaggtcggagggatcacgagacacagaacctggaggggtgtgcacgctggcaggtggcctctgcggcaattgcctcaccctgaggacatcagcagtcagcctgctcagagcgggggtgctggagcgcgtgcagacacagctcttccggagcagccttcaccttctctctgggatcagtgtccggctggccgacgtggcatttgctgaccgaatgctcatagaggttgacccccacagggtcacgcaggactcggacactgccctggaaacatggatggacaagggcttttggccacaggtgtgggtgtcctgttggaggagggcttgtttggagaagggaggctggctgggggagaaacccggatcccgctgcatctccgcgcctgtgggtgcatgtcgcgtgctcatctgttgcacacagctcactcgtatgtcctgcactggtacatgcatctgtaatacagtttctacgtctatttaaggctaggagccgaatgtgccccattgtcagtgggtccacgtttctccccggctcctctgggctaaggcagtgtggcccgaagcttaaaaagttactcggtactgtttttaagaacacttttatagagttagtggaaggcaagttaagagccaatcactgatccccaagtgtttcttgagcatctggtctggggggaccactttgatcggacccacccttggaaagctcaggggtaggcccaggtgggatgctcaccctgtcactgagggttttggttggcatcgttgtttttgaatgtagcacaagcgatgagcaaactctataagagtgttttaaaaattaacttcccaggaagtgagttaaaaacaataaaagccctttcttgagttaaaaagaaaaaaaaaaaaaaaaaaaaaaaaaaaaaaa

SEQ ID NO: 54 - protein, 505 aa, Homo sapiens
MEGGPAVCCQDPRAELVERVAAIDVTHLEEADGGPEPTRNGVDPPPRARAASVIPGSTSRLLPARPSLSARKLSLQERPAGSYLEAQAGPYATGPASHISPRAWRRPTIESHHVAISDAEDCVQLNQYKLQSEIGKGAYGVVRLAYNESEDRHYAMKVLSKKKLLKQYGFPRRPPPRGSQAAQGGPAKQLLPLERVYQEIAILKKLDHVNVVKLIEVLDDPAEDNLYLVFDLLRKGPVMEVPCDKPFSEEQARLYLRDVILGLEYLHCQKIVHRDIKPSNLLLGDDGHVKIADFGVSNQFEGNDAQLSSTAGTPAFMAPEAISDSGQSFSGKALDVWATGVTLYCFVYGKCPFIDDFILALHRKIKNEPVVFPEEPEISEELKDLILKMLDKNPETRIGVPDIKLHPWVTKNGEEPLPSEEEHCSVVEVTEEEVKNSVRLIPSWTTVILVKSMLRKRSFGNPFEPQARREERSMSAPGNLLVKEGFGEGGKSPELPGVQEDEAAS

SEQ ID NO: 55 - DNA, 3529 bp, Homo sapiens
  source 1..3529: mol_type = genomic DNA
agcagaacagagtatgcaatttgggaagctgtggtgtggctgcagtggagagttcccaacaaggctacgcagaagaacccccttgactgaagcaatggaggggggtccagctgtctgctgccaggatcctcgggcagagctggtagaacgggtggcagccatcgatgtgactcacttggaggaggcagatggtggcccagagcctactagaaacggtgtggaccccccaccacgggccagagctgcctctgtgatccctggcagtacttcaagactgctcccagcccggcctagcctctcagccaggaagctttccctacaggagcggccagcaggaagctatctggaggcgcaggctgggccttatgccacggggcctgccagccacatctccccccgggcctggcggaggcccaccatcgagtcccaccacgtggccatctcagatgcagaggactgcgtgcagctgaaccagtacaagctgcagagtgagattggcaagggtgcctacggtgtggtgaggctggcctacaacgaaagtgaagacagacactatgcaatgaaagtcctttccaaaaagaagttactgaagcagtatggctttccacgtcgccctcccccgagagggtcccaggctgcccagggaggaccagccaagcagctgctgcccctggagcgggtgtaccaggagattgccatcctgaagaagctggaccacgtgaatgtggtcaaactgatcgaggtcctggatgacccagctgaggacaacctctatttggtgtttgacctcctgagaaaggggcccgtcatggaagtgccctgtgacaagcccttctcggaggagcaagctcgcctctacctgcgggacgtcatcctgggcctcgagtacttgcactgccagaagatcgtccacagggacatcaagccatccaacctgctcctgggggatgatgggcacgtgaagatcgccgactttggcgtcagcaaccagtttgaggggaacgacgctcagctgtccagcacggcgggaaccccagcattcatggcccccgaggccatttctgattccggccagagcttcagtgggaaggccttggatgtatgggccactggcgtcacgttgtactgctttgtctatgggaagtgcccattcatcgacgatttcatcctggccctccacaggaagatcaagaatgagcccgtggtgtttcctgaggagccagaaatcagcgaggagctcaaggacctgatcctgaagatgttagacaagaatcccgagacgagaattggggtgccagacatcaagttgcacccttgggtgaccaagaacggggaggagccccttccttcggaggaggagcactgcagcgtggtggaggtgacagaggaggaggttaagaactcagtcaggctcatccccagctggaccacggtgatcctggtgaagtccatgctgaggaagcgttcctttgggaacccgtttgagccccaagcacggagggaagagcgatccatgtctgctccaggaaacctactggtgaaagaagggtttggtgaagggggcaagagcccagagctccccggcgtccaggaagacgaggctgcatcctgagcccctgcatgcacccagggccacccggcagcacactcatcccgcgcctccagaggcccacccctcatgcaacagccgcccccgcaggcagggggctggggactgcagccccactcccgcccctcccccatcgtgctgcatgacctccacgcacgcacgtccagggacagactggaatgtatgtcatttggggtcttgggggcagggctcccacgaggccatcctcctcttcttggacctccttggcctgacccattctgtggggaaaccgggtgcccatggagcctcagaaatgccacccggctggttggcatggcctggggcaggaggcagaggcaggagaccaagatggcaggtggaggccaggcttaccacaacggaagagacctcccgctggggccgggcaggcctggctcagctgccacaggcatatggtggagaggggggtaccctgcccaccttggggtggtggcaccagagctcttgtctattcagacgctggtatgggggctcggacccctcactggggacagggccagtgttggagaattctgattccttttttgttgtcttttacttttgtttttaacctgggggttcggggagaggccctgcttgggaacatctcacgagctttcctacatcttccgtggttcccagcacagcccaagattatttggcagccaagtggatggaactaactttcctggactgtgtttcgcattcggcgttatctggaaagtggactgaacggaatcaagctctgagcagaggcctgaagcggaagcaccacatcgtccctgcccatctcactctctcccttgatgatgcccctagagctgaggctggagaagacaccagggctgactttgaccgagggccatggacgcgacaggcctgtggccctgcgcatgctgaaataactggaacccagcctctcctcctacaccggcctacccatctgggcccaagagctgcactcacactcctacaacgaaggacaaactgtccaggtcggagggatcacgagacacagaacctggaggggtgtgcacgctggcaggtggcctctgcggcaattgcctcaccctgaggacatcagcagtcagcctgctcagagcgggggtgctggagcgcgtgcagacacagctcttccggagcagccttcaccttctctctgggatcagtgtccggctggccgacgtggcatttgctgaccgaatgctcatagaggttgacccccacagggtcacgcaggactcggacactgccctggaaacatggatggacaagggcttttggccacaggtgtgggtgtcctgttggaggagggcttgtttggagaagggaggctggctgggggagaaacccggatcccgctgcatctccgcgcctgtgggtgcatgtcgcgtgctcatctgttgcacacagctcactcgtatgtcctgcactggtacatgcatctgtaatacagtttctacgtctatttaaggctaggagccgaatgtgccccattgtcagtgggtccacgtttctccccggctcctctgggctaaggcagtgtggcccgaagcttaaaaagttactcggtactgtttttaagaacacttttatagagttagtggaaggcaagttaagagccaatcactgatccccaagtgtttcttgagcatctggtctggggggaccactttgatcggacccacccttggaaagctcaggggtaggcccaggtgggatgctcaccctgtcactgagggttttggttggcatcgttgtttttgaatgtagcacaagcgatgagcaaactctataagagtgttttaaaaattaacttcccaggaagtgagttaaaaacaataaaagccctttcttgagttaaaaagaaaaaaaaaaaaaaaaaaaaaaaaaaaaaaa

SEQ ID NO: 56 - protein, 532 aa, Homo sapiens
MQFGKLWCGCSGEFPTRLRRRTPLTEAMEGGPAVCCQDPRAELVERVAAIDVTHLEEADGGPEPTRNGVDPPPRARAASVIPGSTSRLLPARPSLSARKLSLQERPAGSYLEAQAGPYATGPASHISPRAWRRPTIESHHVAISDAEDCVQLNQYKLQSEIGKGAYGVVRLAYNESEDRHYAMKVLSKKKLLKQYGFPRRPPPRGSQAAQGGPAKQLLPLERVYQEIAILKKLDHVNVVKLIEVLDDPAEDNLYLVFDLLRKGPVMEVPCDKPFSEEQARLYLRDVILGLEYLHCQKIVHRDIKPSNLLLGDDGHVKIADFGVSNQFEGNDAQLSSTAGTPAFMAPEAISDSGQSFSGKALDVWATGVTLYCFVYGKCPFIDDFILALHRKIKNEPVVFPEEPEISEELKDLILKMLDKNPETRIGVPDIKLHPWVTKNGEEPLPSEEEHCSVVEVTEEEVKNSVRLIPSWTTVILVKSMLRKRSFGNPFEPQARREERSMSAPGNLLVKEGFGEGGKSPELPGVQEDEAAS

SEQ ID NO: 57 - DNA, 2535 bp, Homo sapiens
  source 1..2535: mol_type = genomic DNA
ctgggccccagcgaggcggtggggcggggcggggcggggcggggcgcgcagcaggagcgagtggggccgcccgccgggccgcggacactgtcgcccggcgcccaggttcccaacaaggctacgcagaagaacccccttgactgaagcaatggaggggggtccagctgtctgctgccaggatcctcgggcagagctggtagaacgggtggcagccatcgatgtgactcacttggaggaggcagatggtggcccagagcctactagaaacggtgtggaccccccaccacgggccagagctgcctctgtgatccctggcagtacttcaagactgctcccagcccggcctagcctctcagccaggaagctttccctacaggagcggccagcaggaagctatctggaggcgcaggctgggccttatgccacggggcctgccagccacatctccccccgggcctggcggaggcccaccatcgagtcccaccacgtggccatctcagatgcagaggactgcgtgcagctgaaccagtacaagctgcagagtgagattggcaagggtgcctacggtgtggtgaggctggcctacaacgaaagtgaagacagacactatgcaatgaaagtcctttccaaaaagaagttactgaagcagtatggctttccacgtcgccctcccccgagagggtcccaggctgcccagggaggaccagccaagcagctgctgcccctggagcgggtgtaccaggagattgccatcctgaagaagctggaccacgtgaatgtggtcaaactgatcgaggtcctggatgacccagctgaggacaacctctatttggccctgcagaaccaggcccagaatatccagttagattcaacaaatatcgccaagccccactccctgcttccctctgagcagcaagacagtggatccacgtgggctgcgcgctcagtgtttgacctcctgagaaaggggcccgtcatggaagtgccctgtgacaagcccttctcggaggagcaagctcgcctctacctgcgggacgtcatcctgggcctcgagtacttgcactgccagaagatcgtccacagggacatcaagccatccaacctgctcctgggggatgatgggcacgtgaagatcgccgactttggcgtcagcaaccagtttgaggggaacgacgctcagctgtccagcacggcgggaaccccagcattcatggcccccgaggccatttctgattccggccagagcttcagtgggaaggccttggatgtatgggccactggcgtcacgttgtactgctttgtctatgggaagtgcccattcatcgacgatttcatcctggccctccacaggaagatcaagaatgagcccgtggtgtttcctgaggagccagaaatcagcgaggagctcaaggacctgatcctgaagatgttagacaagaatcccgagacgagaattggggtgccagacatcaagttgcacccttgggtgaccaagaacggggaggagccccttccttcggaggaggagcactgcagcgtggtggaggtgacagaggaggaggttaagaactcagtcaggctcatccccagctggaccacggtgatcctggtgaagtccatgctgaggaagcgttcctttgggaacccgtttgagccccaagcacggagggaagagcgatccatgtctgctccaggaaacctactggtgtaagtactggtgggccagggactgccgggcactccctggagttgggtggggaggtctgaggcccatcctcccactctcactgtcgttgggccaaggccagagcctggggacttggccaggtctcggtgttggccccatttgcatctctgtccccaaggttagtcggggctagaagggaccttttgggcccagctcttgcttcattcctggggccagcatccctcacacacacacttccagggatgaggagctcacgcagcccctccatgggacaggaagacccttcttccatgcagcttgatgtcactctctcactgggtccagcccctctggggcttcaaatctgtggccccctcagcccttggcagcctggcagaggtttgcagacaggctgatgttggcttcctgtaggaggctggcgggctgtagaggaggggtgctggcccctctgcctggccctggggactgttggctgctctcccaagtggcccaggctgcctgcagccattgctggggctctgtgcccagtcagcactttgtgagtgcttgttcagtgagtaagcagggacaggctggccggtggaccacgggagaggaacccgcattggccgagggctccctatggtgagccacgcctgtgggttcaccacctcctaggagggtccagaaaagcagctccccaagcctgtgcgcctcgtcctcagcagatccaccttcttcactataataaaagccagtctgggatgctaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 58 - protein, 520 aa, Homo sapiens
MEGGPAVCCQDPRAELVERVAAIDVTHLEEADGGPEPTRNGVDPPPRARAASVIPGSTSRLLPARPSLSARKLSLQERPAGSYLEAQAGPYATGPASHISPRAWRRPTIESHHVAISDAEDCVQLNQYKLQSEIGKGAYGVVRLAYNESEDRHYAMKVLSKKKLLKQYGFPRRPPPRGSQAAQGGPAKQLLPLERVYQEIAILKKLDHVNVVKLIEVLDDPAEDNLYLALQNQAQNIQLDSTNIAKPHSLLPSEQQDSGSTWAARSVFDLLRKGPVMEVPCDKPFSEEQARLYLRDVILGLEYLHCQKIVHRDIKPSNLLLGDDGHVKIADFGVSNQFEGNDAQLSSTAGTPAFMAPEAISDSGQSFSGKALDVWATGVTLYCFVYGKCPFIDDFILALHRKIKNEPVVFPEEPEISEELKDLILKMLDKNPETRIGVPDIKLHPWVTKNGEEPLPSEEEHCSVVEVTEEEVKNSVRLIPSWTTVILVKSMLRKRSFGNPFEPQARREERSMSAPGNLLV

SEQ ID NO: 59 - DNA, 1153 bp, Homo sapiens
  source 1..1153: mol_type = genomic DNA
gaaaacaccaaatcaaccataggtccaagaacaattgtctctggacggcagctatgcgactcaccgtgctgtgtgctgtgtgcctgctgcctggcagcctggccctgccgctgcctcaggaggcgggaggcatgagtgagctacagtgggaacaggctcaggactatctcaagagattttatctctatgactcagaaacaaaaaatgccaacagtttagaagccaaactcaaggagatgcaaaaattctttggcctacctataactggaatgttaaactcccgcgtcatagaaataatgcagaagcccagatgtggagtgccagatgttgcagaatactcactatttccaaatagcccaaaatggacttccaaagtggtcacctacaggatcgtatcatatactcgagacttaccgcatattacagtggatcgattagtgtcaaaggctttaaacatgtggggcaaagagatccccctgcatttcaggaaagttgtatggggaactgctgacatcatgattggctttgcgcgaggagctcatggggactcctacccatttgatgggccaggaaacacgctggctcatgcctttgcgcctgggacaggtctcggaggagatgctcacttcgatgaggatgaacgctggacggatggtagcagtctagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcattcagaaactatatggaaagagaagtaattcaagaaagaaatagaaacttcaggcagaacatccattcattcattcattggattgtatatcattgttgcacaatcagaattgataagcactgttcctccactccatttagcaattatgtcacccttttttattgcagttggtttttgaatgtctttcactccttttaaggataaactcctttatggtgtgactgtgtcttattcatctatacttgcagtgggtagatgtcaataaatgttacatacacaaataaataaaatgtttattccatggtaaatttaaaaaaaaaaaaaaaaaaaaaaaaaaaaa

SEQ ID NO: 60 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 61 - DNA, 451 bp, Homo sapiens
  source 1..451: mol_type = genomic DNA
gggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctgctggcccgttcgcccctcccggggacctgcggcgggtcgcctgcccagcccccgaaccccgcctggaggccgcggtcggcccggggcttctccggaggcacccactgccaccgcgaagagttgggctctgtcagccgcgggtctctcgggggcgagggcgaggttcaggcctttcaggccgcaggaagaggaacggagcgagtccccgcgcgcggcgcgattccctgagctgtgggacgtgcacccaggactcggctcacacatgc